(19) United States
(12) Patent Application Publication
(10) Pub. No.: US 2026/0041336 A1
(43) Pub. Date: Feb. 12, 2026

(54) PLATELET REACTIVITY EXPRESSION SCORE PREDICTS CARDIOVASCULAR RISK

(71) Applicant: New York University, New York, NY (US)

(72) Inventors: Jeffrey BERGER, Scarsdale, NY (US); Tessa BARRETT, New York, NY (US)

(73) Assignee: New York University, New York, NY (US)

(21) Appl. No.: 19/292,550

(22) Filed: Aug. 6, 2025

Related U.S. Application Data
(60) Provisional application No. 63/679,924, filed on Aug. 6, 2024.

(51) Int. Cl. A61B 5/145; A61B 5/00; G01N 33/53; G01N 33/86

(52) U.S. Cl. CPC A61B 5/145; A61B 5/7282; G01N 33/53; G01N 33/86

(57) ABSTRACT

The present disclosure provides methods for determining whether a subject has platelet hyperreactivity. Further provided are methods for determining risk of developing a platelet-mediated cardiovascular and/or limb event in a subject, as well as determining whether a subject has a condition associated with platelet hyperreactivity. Still further provided are methods for preventing a cardiovascular and/or limb event in a subject in need thereof; treating or preventing a condition associated with platelet hyperreactivity in a subject in need thereof; and, monitoring the progression of a condition associated with platelet hyperreactivity in a subject diagnosed with the condition. Methods for determining the effect of an antiplatelet treatment on development of a condition associated with platelet hyperreactivity in a subject diagnosed with the condition, and identifying an antiplatelet treatment useful for slowing down the progression or treating a condition associated with platelet hyperreactivity in a subject diagnosed with the condition are also provided.

### CROSS-REFERENCE TO RELATED APPLICATIONS

This application claims priority to U.S. Provisional Application No. 63/679,924, filed Aug. 6, 2024, the contents of which is incorporated by reference herein in its entirety.

### STATEMENT REGARDING FEDERALLY SPONSORED RESEARCH

This invention was made with government support under R01 HL114978 and R35 HL144993 awarded by the National Institutes of Health. The government has certain rights in the invention.

### FIELD OF THE INVENTION

The present disclosure relates to methods for determining whether a subject has platelet hyperreactivity, methods for determining risk of developing a platelet-mediated cardiovascular and/or limb event in a subject, as well as methods for determining whether a subject has a condition associated with platelet hyperreactivity. The present disclosure also relates to methods for preventing a cardiovascular and/or limb event in a subject in need thereof; treating or preventing a condition associated with platelet hyperreactivity in a subject; and, methods for monitoring the progression of a condition associated with platelet hyperreactivity in a subject diagnosed with the condition. The present disclosure also relates to methods for determining the effect of an antiplatelet treatment on development of a condition associated with platelet hyperreactivity in a subject diagnosed with the condition, and methods for identifying an antiplatelet treatment useful for slowing down the progression or treating a condition associated with platelet hyperreactivity in a subject diagnosed with the condition.

### BACKGROUND

Platelets play a key role in atherogenesis and progression to thrombosis (1-3). A landmark study published in 1990 reported that increased platelet activity, measured by spontaneous platelet aggregation following a myocardial infarction (MI) off all antiplatelet therapy, was independently associated with long-term mortality and cardiovascular (CV) events (3). Since then, many clinical studies have demonstrated the importance of platelet activity and CV risk (4-8), with multiple studies demonstrating the ability of platelet aggregation measures to identify populations with a hyperreactive platelet phenotype (9,10).

Various technical barriers limit the routine clinical assessment of platelet aggregation responses to assess platelet hyperreactivity and CV risk (11-13). Transcriptome analyses of platelets are increasingly used to discover novel aspects of platelet biology, as diagnostic and prognostic markers, and for therapeutic development efforts (4,14). Several small platelet studies have characterized the transcriptome associated with platelet activity (15-17). More recently, platelet transcriptomic profiling has emerged as an innovative way to detect and track the prevalence and progression of certain malignancies (14,18,19). Technological advancements have enhanced collective understanding of the transcriptome and, coupled with bioinformatics, have fundamentally advanced transcriptomic-based tools to inform disease diagnosis and prognosis (e.g., cardiac allograft rejection (20), detect respiratory viruses (21), and diagnose cancer (22)).

Peripheral artery disease (PAD) affects approximately 8.5 million people over 40 years of age in the United States and 238 million globally (23). Platelets mediate, in part, the pathogenesis of PAD, and platelet hyperreactivity is associated with PAD prevalence, severity, progression, and CV risk (4,24,25). Despite medical and surgical therapy advances, PAD remains associated with significant morbidity and mortality (26). Accordingly, there is an unmet need in the art for identifying individuals with platelet hyperreactivity and, therefore, significant CV risk.

### SUMMARY OF THE INVENTION

As specified in the Background section above, there is a great need in the art for identifying individuals with platelet hyperreactivity and, therefore, significant cardiovascular CV risk. The present application addresses these and other needs.

In one aspect, provided herein is a method for determining whether a subject has platelet hyperreactivity, the method comprising:
a. determining in a blood sample collected from the subject expression level of at least 20, at least 30, at least 40, at least 50, at least 60, at least 70, or all 76 genes selected from the following genes: AC006378.2, AC087481.3, AC106795.2, AC112200.2, AC131971.1, AC243829.1, ACTR3C, A L589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
b. calculating a Platelet Reactivity Expression Score (PRESS) for the sample as a z-scored predictive probability based on weighted expression values of the genes determined in step (a); and
c. determining that i) the subject has platelet hyperreactivity when the score is higher than a median PRESS score of a control cohort; or ii) the subject does not have platelet hyperreactivity when the score is lower than or equal to the median PRESS score of a control cohort.

In some embodiments, when the subject is determined to have platelet hyperreactivity, the method further comprises determining severity of platelet hyperreactivity in the subject based at least in part on the score calculated in step (b), wherein a higher score indicates more severe platelet hyperreactivity. In some embodiments, the subject has not been previously diagnosed with a cardiovascular disease. In some embodiments, the subject has been previously diagnosed with a cardiovascular disease.

In a further aspect, provided herein is a method for determining risk of developing a platelet-mediated cardiovascular and/or limb event in a subject, the method comprising:
a. determining in a blood sample collected from the subject expression level of at least 20, at least 30, at least 40, at least 50, at least 60, at least 70, or all 76 genes selected from the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
b. calculating a Platelet Reactivity Expression Score (PRESS) for the sample as a z-scored predictive probability based on weighted expression values of the genes determined in step (a); and
c. determining that i) the subject is at risk of developing a cardiovascular and/or limb event when the score is higher than a median PRESS score of a control cohort; or ii) the subject is not at risk of developing a cardiovascular and/or limb event when the score is lower than or equal to the median PRESS score of a control cohort.

In some embodiments, the subject has not been previously diagnosed with a cardiovascular disease. In some embodiments, the subject has been previously diagnosed with a cardiovascular disease.

In another aspect, provided herein is a method for determining whether a subject has a condition associated with platelet hyperreactivity, the method comprising:
a. determining in a blood sample collected from the subject expression level of at least 20, at least 30, at least 40, at least 50, at least 60, at least 70, or all 76 genes selected from the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
b. calculating a Platelet Reactivity Expression Score (PRESS) for the sample as a z-scored predictive probability based on weighted expression values of the genes determined in step (a); and
c. determining that i) the subject has a condition associated with platelet hyperreactivity when the score is higher than a median PRESS score of a control cohort; or ii) the subject does not have a condition associated with platelet hyperreactivity when the score is lower than or equal to the median PRESS score of a control cohort.

In some embodiments, the method further comprises administering to the subject one or more antiplatelet treatments.

In some embodiments, the subject has not been previously diagnosed with a cardiovascular disease. In some embodiments, the subject has been previously diagnosed with a cardiovascular disease.

In a further aspect, provided herein is a method for preventing a cardiovascular and/or limb event in a subject in need thereof, the method comprising:
a. determining in a blood sample collected from the subject expression level of at least 20, at least 30, at least 40, at least 50, at least 60, at least 70, or all 76 genes selected from the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
b. calculating a Platelet Reactivity Expression Score (PRESS) for the sample as a z-scored predictive probability based on weighted expression values of the genes determined in step (a); and
c. administering to the subject one or more antiplatelet treatments when the score is higher than a median PRESS score of a control cohort.

In some embodiments, the subject has not been previously diagnosed with a cardiovascular disease. In some embodiments, the subject has been previously diagnosed with a cardiovascular disease.

In another aspect, provided herein is a method for treating or preventing a condition associated with platelet hyperreactivity in a subject in need thereof, the method comprising:
a. determining in a blood sample collected from the subject expression level of at least 20, at least 30, at least 40, at least 50, at least 60, at least 70, or all 76 genes selected from the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
b. calculating a Platelet Reactivity Expression Score (PRESS) for the sample as a z-scored predictive probability based on weighted expression values of the genes determined in step (a); and
c. administering to the subject one or more antiplatelet treatments when the score is higher than a median PRESS score of a control cohort.

In some embodiments, the control cohort is a cohort of patients with peripheral artery disease (PAD).

In some embodiments, the median PRESS score of a control cohort is about 0.38.

In a further aspect, provided herein is a method for monitoring the progression of a condition associated with platelet hyperreactivity in a subject diagnosed with said condition, the method comprising:
a. determining in two or more blood samples collected from the subject at spaced apart time points expression level of at least 20, at least 30, at least 40, at least 50, at least 60, at least 70, or all 76 genes selected from the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
b. calculating a Platelet Reactivity Expression Score (PRESS) for each sample as a z-scored predictive probability based on weighted expression values of the genes determined in step (a); comparing the scores calculated in step (b) between earlier and later collected samples; and
c. determining that (i) the condition in the subject has progressed when the score is increased in the later collected sample(s) as compared to the earlier collected sample(s), or (ii) the condition in the subject has not progressed when the score is not increased in the later collected sample(s) as compared to the earlier collected sample(s).

In another aspect, provided herein is a method for determining the effect of an antiplatelet treatment on development of a condition associated with platelet hyperreactivity in a subject diagnosed with said condition, the method comprising:
a. determining in blood samples collected from the subject prior to and after administering the antiplatelet treatment expression level of at least 20, at least 30, at least 40, at least 50, at least 60, at least 70, or all 76 genes selected from the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
b. calculating a Platelet Reactivity Expression Score (PRESS) for each sample as a z-scored predictive probability based on weighted expression values of the genes determined in step (a);
c. comparing the scores calculated in step (b) between samples collected from the subject prior to and after administering the antiplatelet treatment; and
d. determining that (i) the antiplatelet treatment is effective when the score is decreased in the sample(s) collected after administering the antiplatelet treatment as compared to the sample(s) collected before administering the antiplatelet treatment, or (ii) the antiplatelet treatment is not effective when the score is the same or increased in the sample(s) collected after administering the antiplatelet treatment as compared to the sample(s) collected before administering the antiplatelet treatment.

In some embodiments, the method comprises prior to step (a) the following steps:
(1) collecting one or more sample(s) from the subject prior to initiation of the antiplatelet treatment,
(2) administering the antiplatelet treatment to the subject, and
(3) collecting one or more sample(s) from the subject in the course of or following the antiplatelet treatment.

In a further aspect, provided herein is a method for identifying an antiplatelet treatment useful for slowing down the progression or treating a condition associated with platelet hyperreactivity in a subject diagnosed with said condition, the method comprising:
(a) determining in blood samples collected from the subject prior to and after administering a test antiplatelet treatment expression level of at least 20, at least 30, at least 40, at least 50, at least 60, at least 70, or all 76 genes selected from the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
(b) calculating a Platelet Reactivity Expression Score (PRESS) for each sample as a z-scored predictive probability based on weighted expression values of the genes determined in step (a);
(c) comparing the scores calculated in step (b) between samples collected from the subject prior to and after administering the test antiplatelet treatment; and
(d) determining that (i) the test antiplatelet treatment is effective for slowing down the progression or treating the condition when the score is decreased in the sample(s) collected after administering the test antiplatelet treatment as compared to the sample(s) collected before administering the test antiplatelet treatment, or (ii) the test antiplatelet treatment is not effective for slowing down the progression or treating the condition when the score is the same or increased in the sample(s) collected after administering the test antiplatelet treatment as compared to the sample(s) collected before administering the test antiplatelet treatment.

In some embodiments, the method comprises prior to step (a) the following steps:
(1) collecting one or more sample(s) from the subject prior to administering the test antiplatelet treatment,
(2) administering the test antiplatelet treatment to the subject, and
(3) collecting one or more sample(s) from the subject after administering the test antiplatelet treatment.

In some embodiments, step (a) comprises determining the expression level of additional genes selected from the genes listed in Table 9.

In some embodiments, step (a) comprises determining the expression level of at least 20, at least 50, at least 100, at least 200, or at least 300 additional genes selected from the genes listed in Table 9.

In some embodiments, step (a) comprises determining the expression level of all 451 genes listed in Table 9.

In some embodiments, the genes are weighted according to the values in Table 10.

In some embodiments, the expression level is determined by measuring mRNA level.

In some embodiments, the mRNA level is determined using RNA-seq, real-time polymerase chain reaction (RT-PCR), Northern blot analysis, or a Ribonuclease protection assay.

In some embodiments, the blood sample comprises isolated platelets.

In some embodiments, the blood sample consists of isolated platelets.

In some embodiments, the platelet hyperreactivity is measured as >60% aggregation in response to submaximal epinephrine (0.4 μM).

In some embodiments, the condition associated with platelet hyperreactivity is cardiovascular disease, systemic lupus erythematosus (SLE), and COVID-19.

In some embodiments, the cardiovascular disease is atherosclerosis, peripheral artery disease (lower extremity atherosclerosis), or coronary artery disease.

In some embodiments, the cardiovascular event is death, myocardial infarction (MI), and/or stroke.

In some embodiments, the limb event is major amputation, acute limb ischemia, or a reintervention of the index limb.

In some embodiments, the reintervention of the index limb comprises a new bypass graft, a jump/interposition graft revision, thrombectomy/thrombolysis, balloon angioplasty, atherectomy, laser treatment and/or stenting or stent/grafting.

In some embodiments, the antiplatelet treatment comprises one or more of aspirin, acadesine, prasugrel, ticagrelor, enilogrel, eptifibatide, cangrelor, anagrelide, anipamil, argatroban, clopidogrel, FR-122047, danaparoid sodium, dazoxiben hydrochloride, diadenosine 5′,5″-P1,P4-tetraphosphate (Ap4A) analogs, defibrotide, dilazep dihydrochloride, 1,2- and 1,3-glyceryl dinitrate, dipyridamole, dopamine and 3-methoxytyramine, efegatran sulfate, enoxaparin sodium, glucagon, Ro-43-8857, L-700,462, ifetroban, ifetroban sodium, iloprost, isocarbacyclin methyl ester, isosorbide-5-mononitrate, itazigrel, ketanserin, BM-13.177, lamifiban, lifarizine, molsidomine, nifedipine, oxagrelate, prostaglandin E (PGE), lexipafant, prostacyclin (PGI2), pyrazines, pyridinol carbamate, abciximab, sulfinpyrazone, BN-50727, BN-52021, CV-4151, E-5510, FK-409, GU-7, KB-2796, KBT-3022, KC-404, KF-4939, OP-41483-, TRK-100, TA-3090, TFC-612 and ZK-36374, 2,4,5,7-tetrathiaoctane, 2,4,5,7-tetrathiaoctane 2,2-dioxide, 2,4,5-trithiahexane, theophylline, pentoxifylline, picotamide, sulotroban, tirofiban, trapidil, trifenagrel, trilinolein, dipyridamole, clofibrate, caffeine, ticlopidine, or an analog or derivative thereof.

In some embodiments, the subject is human.

In some embodiments, the present disclosure provides a non-transitory computer-readable medium configured to communicate with one or more processor(s) of a computing device. In some embodiments, the non-transitory computer-readable medium includes instructions thereon that, when executed by the processor(s), cause the computing device to perform any of the methods disclosed herein.

The present disclosure also provides a computing system comprising: a memory; one or more processors; and program instructions stored in the memory that, when executed by the one or more processors, cause the computing system to:
(a) determine, in a blood sample collected from a subject, expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A; and
(b) calculate a Platelet Reactivity Expression Score (PRESS) for the blood sample as a z-scored predictive probability based on weighted expression values calculated from the expression level of the genes determined in process step (a).

In some embodiments, the computing systems described herein, further comprise instructions stored in the memory that, when executed by the one or more processors, cause the computing system to determine that:
i) the subject has platelet hyperreactivity when the score is higher than a median PRESS score of a control cohort; or
ii) the subject does not have platelet hyperreactivity when the score is lower than or equal to the median PRESS score of a control cohort.

In some embodiments, the computing systems described herein, further comprise instructions stored in the memory that, when executed by the one or more processors, cause the computing system to determine that:
i) the subject is at risk of developing a cardiovascular and/or limb event when the score is higher than a median PRESS score of a control cohort; or
ii) the subject is not at risk of developing a cardiovascular and/or limb event when the score is lower than or equal to the median PRESS score of a control cohort.

In some embodiments, the computing systems described herein, further comprise instructions stored in the memory that, when executed by the one or more processors, cause the computing system to determine that:
i) the subject has a condition associated with platelet hyperreactivity when the score is higher than a median PRESS score of a control cohort; or
ii) the subject does not have a condition associated with platelet hyperreactivity when the score is lower than or equal to the median PRESS score of a control cohort.

In some embodiments, the computing systems described herein, further comprise an input/output (I/O) device; and instructions stored in the memory that, when executed by the one or more processors, cause the computing system to:
(a) output, for display on the I/O device, a recommendation to administer, to the subject, one or more antiplatelet treatments when the score is higher than a median PRESS score of a control cohort.

In some embodiments, the present disclosure provides computing systems comprising: a memory; one or more processors; and program instructions stored in the memory that, when executed by the one or more processors, cause the computing system to calculate a Platelet Reactivity Expression Score (PRESS) for a blood sample collected from a subject. In some embodiments, the PRESS is calculated as a z-scored predictive probability based on weighted expression values calculated from the expression level of the following genes in the blood sample: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A.

In some embodiments, the computing systems described herein, further comprise instructions stored in the memory that, when executed by the one or more processors, cause the computing system to determine that:
i) the subject has platelet hyperreactivity when the score is higher than a median PRESS score of a control cohort; or
ii) the subject does not have platelet hyperreactivity when the score is lower than or equal to the median PRESS score of a control cohort.

In some embodiments, the computing systems described herein, further comprise instructions stored in the memory that, when executed by the one or more processors, cause the computing system to determine that:
(i) the subject is at risk of developing a cardiovascular event when the score is higher than a median PRESS score of a control cohort; or
(ii) the subject is not at risk of developing a cardiovascular event when the score is lower than or equal to the median PRESS score of a control cohort.

In some embodiments, the computing systems described herein, further comprise instructions stored in the memory that, when executed by the one or more processors, cause the computing system to determine that:
i) the subject has a condition associated with platelet hyperreactivity when the score is higher than a median PRESS score of a control cohort; or
ii) the subject does not have a condition associated with platelet hyperreactivity when the score is lower than or equal to the median PRESS score of a control cohort.

In some embodiments, the computing systems described herein, further comprise an input/output (I/O) device; and instructions stored in the memory that, when executed by the one or more processors, cause the computing system to:
(a) output, for display on the I/O device, a recommendation to administer, to the subject, one or more antiplatelet treatments when the score is higher than a median PRESS score of a control cohort.

In some embodiments, the present disclosure provides a computing system for monitoring the progression of a condition associated with platelet hyperreactivity in a subject diagnosed with said condition, comprising: a memory; one or more processors; and program instructions stored in the memory that, when executed by the one or more processors, cause the computing system to:
(a) determine in two or more blood samples collected from the subject at spaced apart time points expression level of the following genes AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
(b) calculate a Platelet Reactivity Expression Score (PRESS) for each sample as a z-scored predictive probability based on weighted expression values of the genes calculated from the expression level determined in process step (a); and
(c) compare the scores calculated in process step (b) between earlier and later collected samples.

In some embodiments, the computing systems described herein, further comprise instructions stored in the memory that, when executed by the one or more processors, cause the computing system to determine that:
(i) the condition in the subject has progressed when the score is increased in the later collected sample(s) as compared to the earlier collected sample(s), or
(ii) the condition in the subject has not progressed when the score is not increased in the later collected sample(s) as compared to the earlier collected sample(s).

In some embodiments, the present disclosure provides computing systems for determining the effect of an antiplatelet treatment on development of a condition associated with platelet hyperreactivity in a subject diagnosed with said condition, comprising: a memory; one or more processors; and program instructions stored in the memory that, when executed by the one or more processors, cause the computing system to:
(a) determine, in blood samples collected from the subject prior to and after administering the antiplatelet treatment, expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIPSK1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
(b) calculate a Platelet Reactivity Expression Score (PRESS) for each sample as a z-scored predictive probability based on weighted expression values calculated from the expression level of the genes determined in process step (a).

In some embodiments, the computing systems described herein, further comprise instructions stored in the memory that, when executed by the one or more processors, cause the computing system to compare the scores calculated in process step (b) between samples collected from the subject prior to and after administering the antiplatelet treatment, determine that:
(i) the antiplatelet treatment is effective when the score is decreased in the sample(s) collected after administering the antiplatelet treatment as compared to the sample(s) collected before administering the antiplatelet treatment, or
(ii) the antiplatelet treatment is not effective when the score is the same or increased in the sample(s) collected after administering the antiplatelet treatment as compared to the sample(s) collected before administering the antiplatelet treatment.

In some embodiments, the present disclosure provides computing systems for identifying an antiplatelet treatment useful for slowing down the progression or treating a condition associated with platelet hyperreactivity in a subject diagnosed with said condition, comprising: a memory; one or more processors; and program instructions stored in the memory that, when executed by the one or more processors, cause the computing system to:

(a) determine in blood samples collected from the subject prior to and after administering a test antiplatelet treatment expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A; and
(b) calculate a Platelet Reactivity Expression Score (PRESS) for each sample as a z-scored predictive probability based on weighted expression values calculated from the expression level of the genes determined in process step (a).

In some embodiments, the computing systems described herein, further comprise instructions stored in the memory that, when executed by the one or more processors, cause the computing system to compare the scores calculated in process step (b) between samples collected from the subject prior to and after administering the test antiplatelet treatment; and determine that:
(i) the test antiplatelet treatment is effective for slowing down the progression or treating the condition when the score is decreased in the sample(s) collected after administering the test antiplatelet treatment as compared to the sample(s) collected before administering the test antiplatelet treatment, or
(ii) the test antiplatelet treatment is not effective for slowing down the progression or treating the condition when the score is the same or increased in the sample(s) collected after administering the test antiplatelet treatment as compared to the sample(s) collected before administering the test antiplatelet treatment.

Also provided herein is a computing system comprising: a memory; one or more processors; and program instructions stored in the memory that, when executed by the one or more processors, cause the computing system to: calculate a first value of a subject as a z-scored predictive probability based on weighted expression values calculated from an expression level of a subset of genes; and output a recommendation to administer, to the subject, one or more antiplatelet treatments when the value is higher than a median value score of a control cohort, wherein the expression level is determined for the following genes in a blood sample of the subject: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIPSK1B, PTMAP10, RAB3GAP2, RACIP4, RAD51AP1, RASA1, RTN1, SEC22A, SEM1, SEPT7P7, SLC3A2, SPARC, SPIN4, SUGTIPI, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A. In some embodiments, the first value is a Platelet Reactivity Expression Score (PRESS).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1F show platelet hyperreactivity and incidence of major adverse cardiac or limb events following lower extremity revascularization. FIG. 1A shows the PACE-PAD trial design. Patients with peripheral artery disease (PAD) undergoing lower extremity revascularization (LER) were recruited into the PACE-PAD study (NCT02106429). Peripheral blood samples were collected immediately before the LER procedure, platelet aggregation was assessed via light transmission aggregometry (n=254), and platelets were isolated for subsequent RNA-sequencing (n=129). Patients were followed for 30 days following LER to assess post-operative CV events, with the primary endpoint a composite of major adverse cardiac or limb event (MACLE; defined by death, myocardial infarction (MI), stroke, major amputation, major or minor reintervention of the index limb). FIG. 1B shows patients who experienced a MACLE within 30 days of LER had higher platelet aggregation responses to 0.4 μM epinephrine before the LER procedure. FIG. 1C shows bimodal distribution of platelet aggregation responses to 0.4 μM epinephrine of PACE-PAD patients. Hyperreactivity was defined as >60% aggregation. FIG. 1D shows PACE-PAD patients whose platelet reactivity at baseline was >60% were significantly more likely to experience MACLE (n=246). FIG. 1E shows influence of platelet aggregation to MACLE incidence. FIG. 1F shows Kaplan-Meier analysis of 30 day MACLE. Platelet aggregation responses above the median were associated with a 2.76-fold increase in MACLE (HR=2.76, p=0.001).

FIGS. 2A-2B show platelet hyperreactivity to 0.4 μM epinephrine and incidence of major adverse cardiac or limb event within 30 days post lower extremity revascularization. FIG. 2A shows the adjusted hazard ratio for major adverse cardiac or limb events (MACLE; defined by death, myocardial infarction (MI), stroke, major amputation, major or minor reintervention of the index limb) at 30 days based on preprocedural platelet aggregation responses to 0.4 μM epinephrine. Multivariable adjustment was performed for age, sex, race/ethnicity, body mass index, diabetes, carotid artery disease, prior stroke, and critical limb ischemia. FIG. 2B shows the sub-group analysis of platelet hyperreactivity to 0.4 μM epinephrine and incidence of MACLE within 30 days post lower extremity revascularization. The adjusted hazard ratio is shown for MACLE at 30 days based on preprocedural platelet aggregation responses to 0.4 μM epinephrine (n=43 hyperreactive, n=203 not hyperreactive). BMI: body mass index, CLI: critical limb ischemia, CAD: coronary artery disease, RCRI: revised cardiac risk index.

FIGS. 3A-3F show the data supporting defining a gene signature of platelet hyperreactivity, Platelet Reactivity Expression Score (PRESS). FIGS. 3A-3B show that platelets were collected from PACE-PAD patients before lower extremity revascularization (LER; n=129) to generate a platelet hyperreactivity gene signature. Platelet RNA-seq and stratification of patients by hyperreactive platelet responses (aggregation responses >60% versus ≤40% to 0.4 μM epinephrine, n=84) were performed. FIGS. 3C-3D show overlap of transcripts differentially expressed (using both DESeq2 Wald test and the Wilcoxon rank-sum test after adjustment for age, sex, and race/ethnicity) between patients with hyperreactive or not hyperreactive platelets (n=796), and transcripts significantly correlated with platelet aggregation responses to 0.4 μM epinephrine (n=2035). Overlapping genes (PRESS gene set; n=451) are comprised of 174 transcripts positively and 277 transcripts negatively associated with platelet hyperreactivity. FIG. 3E shows a boxplot showing the platelet hyperreactivity signature score in 10-fold cross-validation training comparing hyperreactive platelet and not hyperreactive platelet patient groups. Boxplots show mean and IQR prediction probabilities following z-score normalization. FIG. 3F shows receiver operating characteristic (ROC) curve of the mean 10-fold cross-validation training of the hyperreactive platelet signature. The shaded areas represent 95% CIs plotted for sensitivity at given in-sample specificities.

FIGS. 4A-4F show validation of the Platelet Reactivity Expression Score (PRESS). FIGS. 4A-4B show that the PRESS signature was validated in an independent cohort of healthy participants with platelet aggregation measured at two separate time points and platelet RNAseq performed (n=35). In this cohort, 14 participants had a hyperreactive platelet phenotype (aggregation >60% in response to epinephrine 0.5 μM). FIG. 4C shows a boxplot showing the performance of the extra-trees classifier ensemble model that was trained on the PRESS gene set within the PACE cohort and externally validated on the independent validation cohort. Boxplots show mean and IQR prediction probabilities following z-score normalization. FIG. 4D shows receiver operating characteristic (ROC) curve of the extra-trees classifier ensemble model validated in the independent validation cohort of healthy participants. The shaded areas represent 95% of CIs plotted for sensitivity at given in-sample specificities. FIG. 4E shows the performance of the PRESS gene set in the independent validation cohort (n=35). FIG. 4F shows the GSEA pathway analysis, incorporating the PRESS gene set of the independent validation cohort, revealed significant enrichment of upregulated and downregulated PRESS in subjects with hyperreactive platelets.

FIGS. 5A-5E show the Platelet Reactivity Expression Score (PRESS) discriminates those at increased risk for MI and a future CV event. FIG. 5A shows GSEA pathway analysis, incorporating PRESS into multiple clinical cohorts or platelet subtypes; Atherosclerosis, systemic lupus erythematosus, COVID-19, and reticulated platelets with platelet RNA-seq revealed significant enrichment of both upregulated and downregulated PRESS. Refer to FIG. 6 for population descriptions. FIG. 5B shows platelet aggregation to 0.4 μM epinephrine stratification based on each individuals PRESS (n=129) in the PACE-PAD cohort. FIG. 5C shows subjects with a PRESS greater than the median have increased platelet aggregation responses to epinephrine at multiple doses (0.1 μM, 0.4 μM, and 2 μM) and time points. FIG. 5D shows PRESS was calculated in a cohort of patients referred for coronary angiography with myocardial infarction (MI) (n=19) relative to patients referred for cardiac catheterization with coronary artery disease without MI (n=9; p=0.0084, t-test). FIG. 5E shows Kaplan-Meier cumulative incidence plot of major adverse cardiac events (death, myocardial infarction, stroke, or major amputation) in a cohort of patients with peripheral artery disease followed for a median of 18 months. After adjustment for age, sex, race/ethnicity, BMI, diabetes, carotid artery disease, prior stroke, and CLI, a PRESS above the median was associated with a 90% increase in a major cardiovascular event (50.7% vs. 31.9% below the median, adjusted HR 1.90, CI 1.07-3.36, p=0.027).

FIG. 6 shows the study populations.

FIG. 7 shows platelet hyperreactivity and Major Adverse Cardiac and Limb Events (MACLE) after excluding participants off antiplatelet therapy.

FIGS. 8A-8D show platelet hyperreactivity in response to a range of epinephrine concentrations. FIGS. 8A-8B show the distribution of aggregation in response to 2 μM (FIG. 8A) and 0.1 μM (FIG. 8B) epinephrine. FIGS. 8C-8D show incidence of MACLE in response to 2 μM (FIG. 8C) and 0.1 μM (FIG. 8D) epinephrine induced platelet aggregation.

FIG. 9 shows gene set enrichment analysis (GSEA) of the PRESS geneset and enriched gene ontology (GO) pathways.

FIG. 10 shows Kaplan-Meier cumulative incidence plot of major adverse cardiac events (death, myocardial infarction, stroke, or major amputation) in a cohort of patients with peripheral artery disease followed for a median of 18 months.

FIG. 11 illustrates a block diagram of an embodiment of a computing device 1100.

FIG. 12 illustrates a block diagram of an embodiment of a computing network 1200 including computing device(s) 1210, server(s) 1220, memory store(s) 1240, and a network 1230 facilitating communication between each.

### DETAILED DESCRIPTION

Platelets are key mediators of atherothrombosis, yet limited tools exist to identify individuals with a hyperreactive platelet phenotype. Developing a platelet hyperreactivity risk score is an innovative approach to identifying individuals with platelet hyperreactivity and, therefore, at significant CV risk. The Platelet Activity and Cardiovascular Events in peripheral artery disease (PACE-PAD) clinical study was designed to (1) prospectively investigate whether in symptomatic PAD patients requiring lower extremity revascularization (LER), platelet activity is associated with incident major adverse cardiovascular and limb events (MACLE) and (2) evaluate whether the platelet transcriptome identifies a platelet hyperreactivity signature predictive of incident CV events. Leveraging platelet activity and platelet transcriptomic measures from this well-phenotyped clinical cohort facilitated the development of a Platelet Reactivity Expression Score (PRESS), which discriminates individuals with a hyperreactive platelet phenotype. Some embodiments of the PRESS-based methods described herein integrate platelet aggregation responses and RNA sequencing.

As illustrated in the Examples section below, among patients with peripheral artery disease (PAD), those with a hyperreactive platelet response (>60% aggregation) to 0.4 μM epinephrine had a higher incidence of the 30-day primary cardiovascular endpoint (37.2% vs. 15.3% in those without hyperreactivity, adjusted HR 2.76, 95% CI 1.5-5.1, p=0.002). PRESS performed well in identifying a hyperreactive phenotype in patients with PAD (AUC [cross-validation] 0.81, 95% CI 0.68 to 0.94, n=84) and in an independent cohort of healthy participants (AUC [validation] 0.77, 95% CI 0.75 to 0.79, n=35). PRESS was associated with acute myocardial infarction, and a higher risk for developing a future CV event. Following multivariable adjustment, PAD individuals with a PRESS score above the median are at higher risk for a future cardiovascular event (adjusted HR 1.90, CI 1.07 to 3.36; p-0.027, n=129, NCT02106429). This study provides an informatics tool that discriminates platelet hyperreactivity, which can serve as a diagnostic tool. This study derives and validates the ability of PRESS to discriminate platelet hyperreactivity and identify those at increased cardiovascular risk. The development of a platelet reactivity expression score opens the possibility for a personalized approach to antithrombotic therapy for cardiovascular risk reduction.

### Methods

The present disclosure provides methods for determining whether a subject has platelet hyperreactivity. In some embodiments, the method for determining whether a subject has platelet hyperreactivity comprises (a) determining the expression level of one or more of the following genes in a blood sample from a subject, including but not limited to, AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NRID2, PAPPA, PIP5K1B, PTMAP10, RAB3GAP2, RAC1P4, RAD51AP1, RASAI, RTN1, SEC22A, SEM1, SEPT7P7, SLC3A2, SPARC, SPIN4, SUGTIP1, SVIL, TCAIM, THNSLI, THUMPD2, TRBV4-1, TSPAN13, UBE4B, VNN1, ZBTB11, ZFR, ZHX1-C8orf76, ZNF460-AS1, and/or ZNF804A. In some embodiments, the method further includes a step (b) comprising calculating a Platelet Reactivity Expression Score (PRESS) for the sample as a z-scored predictive probability based on weighted expression values calculated from the expression level of the genes determined in step (a). In some embodiments, the method includes determining that i) the subject has platelet hyperreactivity when the score is higher than a median PRESS score of a control cohort; or ii) the subject does not have platelet hyperreactivity when the score is lower than or equal to the median PRESS score of a control cohort.

In some embodiments, when the subject is determined to have platelet hyperreactivity, the method further comprises determining severity of platelet hyperreactivity in the subject based at least in part on the score calculated as disclosed above, wherein a higher score indicates more severe platelet hyperreactivity. In some embodiments, the subject has not been previously diagnosed with a cardiovascular disease. In some embodiments, the subject has been previously diagnosed with a cardiovascular disease.

In some embodiments, the method of determining platelet hyperreactivity includes determining PRESS score and comparing the score to a median PRESS score of a control cohort. In some embodiments, the control cohort is a cohort of patients with peripheral artery disease (PAD).

In some embodiments, the median PRESS score of the control cohort is greater than 0.37. In some embodiments, the median PRESS score is about 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or more.

In some embodiments, when the subject is determined to have platelet hyperreactivity, the method further comprises administering to the subject one or more antiplatelet treatments.

In some embodiments, the methods of the present disclosure comprise determining risk of developing a platelet-mediated cardiovascular and/or limb event in a subject. In some embodiments, the method comprises (a) determining in a blood sample collected from the subject, expression level of genes including but not limited to, AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RACIP4, RAD51AP1, RASAI, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A. In some embodiments, the method further includes a step (b) comprising calculating a Platelet Reactivity Expression Score (PRESS) for the sample as a z-scored predictive probability based on weighted expression values calculated from the expression level of the genes determined in step (a). In some embodiments, the method of determining risk of developing a platelet-mediated cardiovascular and/or limb event in a subject further includes determining that i) the subject is at risk of developing a cardiovascular and/or limb event when the score is higher than a median PRESS score of a control cohort; or ii) the subject is not at risk of developing a cardiovascular and/or limb event when the score is lower than or equal to the median PRESS score of a control cohort.

In some embodiments, the method of determining risk of developing a platelet-mediated cardiovascular and/or limb event in a subject includes determining PRESS score and comparing the score to a median PRESS score of a control cohort. In some embodiments, the control cohort is a cohort of patients with peripheral artery disease (PAD).

In some embodiments, the median PRESS score of the control cohort is greater than 0.37. In some embodiments, the median PRESS score is about 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or more.

In some embodiments, when the subject is determined to be at risk of developing a platelet-mediated cardiovascular and/or limb event, the method further comprises administering to the subject one or more antiplatelet treatments.

The present disclosure also provides a method for determining whether a subject has a condition associated with platelet hyperreactivity. In some embodiments, the method comprises determining in a blood sample collected from the subject, expression level of genes including but not limited to AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIPSK1B, PTMAP10, RAB3GAP2, RACIP4, RAD51AP1, RASAI, RTN1, SEC22A, SEM1, SEPT7P7, SLC3A2, SPARC, SPIN4, SUGTIP1, SVIL, TCAIM, THNSLI, THUMPD2, TRBV4-1, TSPAN13, UBE4B, VNN1, ZBTB11, ZFR, ZHX1-C8orf76, ZNF460-AS1, and ZNF804A. In some embodiments, the method of determining whether a subject has a condition associated with platelet hyperreactivity includes a step (b) comprising calculating a Platelet Reactivity Expression Score (PRESS) for the sample as a z-scored predictive probability based on weighted expression values calculated from the expression level of the genes determined in step (a). In some embodiments, the method includes determining that i) the subject has a condition associated with platelet hyperreactivity when the score is higher than a median PRESS score of a control cohort; or ii) the subject does not have a condition associated with platelet hyperreactivity when the score is lower than or equal to the median PRESS score of a control cohort.

In some embodiments, the method of determining whether a subject has a condition associated with platelet hyperreactivity includes determining PRESS score and comparing the score to a median PRESS score of a control cohort. In some embodiments, the control cohort is a cohort of patients with peripheral artery disease (PAD).

In some embodiments, the median PRESS score of the control cohort is greater than 0.37. In some embodiments, the median PRESS score is about 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or more.

In some embodiments, the method of determining whether a subject has a condition associated with platelet hyperreactivity further comprises administering to the subject one or more antiplatelet treatments.

The present disclosure also provides methods for preventing a cardiovascular and/or limb event in a subject in need thereof. Such methods comprise: (a) determining in a blood sample collected from the subject expression level of one or more of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RACIP4, RAD51AP1, RASAI, RTN1, SEC22A, SEM1, SEPT7P7, SLC3A2, SPARC, SPIN4, SUGTIP1, SVIL, TCAIM, THNSLI, THUMPD2, TRBV4-1, TSPAN13, UBE4B, VNN1, ZBTB11, ZFR, ZHXI-C8orf76, ZNF460-AS1, and ZNF804A. In some embodiments, the methods further include a step (b) of calculating a Platelet Reactivity Expression Score (PRESS) for the sample as a z-scored predictive probability based on weighted expression values of the genes determined in step (a). In some embodiments, the methods further include administering to the subject one or more antiplatelet treatments when the score is higher than a median PRESS score of a control cohort.

In some embodiments, the method of preventing a cardiovascular and/or limb event in a subject in need thereof includes determining PRESS score and comparing the score to a median PRESS score of a control cohort. In some embodiments, the control cohort is a cohort of patients with peripheral artery disease (PAD).

In some embodiments, the median PRESS score of the control cohort is greater than 0.37. In some embodiments, the median PRESS score is about 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or more.

The present disclosure also provides a method for treating or preventing a condition associated with platelet hyperreactivity in a subject in need thereof. In some embodiments, the method comprises (a) determining in a blood sample collected from the subject, expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIPSK1B, PTMAP10, RAB3GAP2, RACIP4, RAD51AP1, RASAI, RTN1, SEC22A, SEM1, SEPT7P7, SLC3A2, SPARC, SPIN4, SUGTIP1, SVIL, TCAIM, THNSLI, THUMPD2, TRBV4-1, TSPAN13, UBE4B, VNN1, ZBTB11, ZFR, ZHX1-C8orf76, ZNF460-AS1, and ZNF804A. In some embodiments, the methods include a step (b) comprising calculating a Platelet Reactivity Expression Score (PRESS) for the sample as a z-scored predictive probability based on weighted expression values calculated from the expression level of the genes determined in step (a) disclosed above. In some embodiments, the methods further include administering to the subject one or more antiplatelet treatments when the score is higher than a median PRESS score of a control cohort.

In some embodiments, the method of treating or preventing a condition associated with platelet hyperreactivity in a subject in a subject in need thereof includes determining PRESS score and comparing the score to a median PRESS score of a control cohort. In some embodiments, the control cohort is a cohort of patients with peripheral artery disease (PAD).

In some embodiments, the median PRESS score of the control cohort is greater than 0.37. In some embodiments, the median PRESS score is about 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or more.

In some embodiments, the methods of monitoring the progression of a condition associated with platelet hyperreactivity in a subject diagnosed with the condition include (a) determining in two or more blood samples collected from the subject at spaced apart time points expression level of the following genes AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A; (b) calculating a Platelet Reactivity Expression Score (PRESS) for each sample as a z-scored predictive probability based on weighted expression values of the genes determined in step (a); comparing the scores calculated in step (b) between earlier and later collected samples; and (c) determining that (i) the condition in the subject has progressed when the score is increased in the later collected sample(s) as compared to the earlier collected sample(s), or (ii) the condition in the subject has not progressed when the score is not increased in the later collected sample(s) as compared to the earlier collected sample(s).

The present disclosure also provides a method for determining the effect of an antiplatelet treatment on development of a condition associated with platelet hyperreactivity in a subject diagnosed with said condition. In some embodiments, the method includes determining in blood samples collected from the subject prior to and after administering the antiplatelet treatment expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NRID2, PAPPA, PIP5K1B, PTMAP10, RAB3GAP2, RAC1P4, RAD51AP1, RASAI, RTN1, SEC22A, SEM1, SEPT7P7, SLC3A2, SPARC, SPIN4, SUGTIP1, SVIL, TCAIM, THNSLI, THUMPD2, TRBV4-1, TSPAN13, UBE4B, VNN1, ZBTB11, ZFR, ZHX1-C8orf76, ZNF460-AS1, and ZNF804A; (b) calculating a Platelet Reactivity Expression Score (PRESS) for each sample as a z-scored predictive probability based on weighted expression values of the genes determined in step (a); (c) comparing the scores calculated in step (b) between samples collected from the subject prior to and after administering the antiplatelet treatment; and (d) determining that (i) the antiplatelet treatment is effective when the score is decreased in the sample(s) collected after administering the antiplatelet treatment as compared to the sample(s) collected before administering the antiplatelet treatment, or (ii) the antiplatelet treatment is not effective when the score is the same or increased in the sample(s) collected after administering the antiplatelet treatment as compared to the sample(s) collected before administering the antiplatelet treatment.

In some embodiments, prior to determining the expression level of the genes, one or more of the following steps may be performed including (1) collecting one or more sample(s) from the subject prior to initiation of the antiplatelet treatment, (2) administering the antiplatelet treatment to the subject, and (3) collecting one or more sample(s) from the subject in the course of or following the antiplatelet treatment.

The present disclosure also provides methods for identifying an antiplatelet treatment useful for slowing down the progression or treating a condition associated with platelet hyperreactivity in a subject diagnosed with the condition. In some embodiments, the method comprises, (a) determining in blood samples collected from the subject prior to and after administering a test antiplatelet treatment expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSLI, THUMPD2, TRBV4-1, TSPAN13, UBE4B, VNN1, ZBTB11, ZFR, ZHX1-C8orf76, ZNF460-AS1, and ZNF804A; (b) calculating a Platelet Reactivity Expression Score (PRESS) for each sample as a z-scored predictive probability based on weighted expression values of the genes determined in step (a); (c) comparing the scores calculated in step (b) between samples collected from the subject prior to and after administering the test antiplatelet treatment; and (d) determining that (i) the test antiplatelet treatment is effective for slowing down the progression or treating the condition when the score is decreased in the sample(s) collected after administering the test antiplatelet treatment as compared to the sample(s) collected before administering the test antiplatelet treatment, or (ii) the test antiplatelet treatment is not effective for slowing down the progression or treating the condition when the score is the same or increased in the sample(s) collected after administering the test antiplatelet treatment as compared to the sample(s) collected before administering the test antiplatelet treatment. In some embodiments, prior to determining the gene expression level, the method includes one or more of the following steps: (1) collecting one or more sample(s) from the subject prior to administering the test antiplatelet treatment, (2) administering the test antiplatelet treatment to the subject, and (3) collecting one or more sample(s) from the subject after administering the test antiplatelet treatment.

In some embodiments, any of the methods disclosed herein comprise determining the expression level of one or more genes listed in Table 1. Table 1 provides the gene names and/or GenBank identifiers for the genes used to determine PRESS as well as their ENSEMBL identifiers.

TABLE 1
PRESS GENES and their ENSEMBL identifiers

| SYMBOL | ENSEMBL ID | SYMBOL | ENSEMBL ID |
|---|---|---|---|
| GABRD | ENSG00000187730 | ANGPT1 | ENSG00000154188 |
| UBE4B | ENSG00000130939 | EMC2 | ENSG00000104412 |
| PGD | ENSG00000142657 | AC104248.1 | ENSG00000253796 |
| AL606491.1 | ENSG00000225643 | ENY2 | ENSG00000120533 |
| PAQR7 | ENSG00000182749 | ZHX1-C8orf76 | ENSG00000259305 |
| ARID1A | ENSG00000117713 | ST3GAL1 | ENSG00000008513 |
| TAF12 | ENSG00000120656 | ZNF517 | ENSG00000197363 |
| SPOCD1 | ENSG00000134668 | FTH1P12 | ENSG00000213362 |
| FAM229A | ENSG00000225828 | ACER2 | ENSG00000177076 |
| BSDC1 | ENSG00000160058 | KLHL9 | ENSG00000198642 |
| STK40 | ENSG00000196182 | IFT74 | ENSG00000096872 |
| GPX7 | ENSG00000116157 | SMU1 | ENSG00000122692 |
| TPI1P1 | ENSG00000226415 | SUGT1P1 | ENSG00000226823 |
| AC118549.1 | ENSG00000036549 | PIP5K1B | ENSG00000107242 |
| SLC25A24 | ENSG00000085491 | ABHD17B | ENSG00000107362 |
| WDR47 | ENSG00000085433 | GADD45G | ENSG00000130222 |
| GNAI3 | ENSG00000065135 | NINJ1 | ENSG00000131669 |
| RHOC | ENSG00000155366 | NCBP1 | ENSG00000136937 |
| VPS45 | ENSG00000136631 | HEMGN | ENSG00000136929 |
| MINDY1 | ENSG00000143409 | CAVIN4 | ENSG00000170681 |
| MLLT11 | ENSG00000213190 | SMC2 | ENSG00000136824 |
| JTB | ENSG00000143543 | PTBP3 | ENSG00000119314 |
| RAB13 | ENSG00000143545 | PAPPA | ENSG00000182752 |
| FAM189B | ENSG00000160767 | AL137024.1 | ENSG00000244757 |
| F11R | ENSG00000158769 | GSN | ENSG00000148180 |
| TOMM40L | ENSG00000158882 | CIZ1 | ENSG00000148337 |
| TADA1 | ENSG00000152382 | TBC1D13 | ENSG00000107021 |
| TIPRL | ENSG00000143155 | LINC00963 | ENSG00000204054 |
| BLZF1 | ENSG00000117475 | FNBP1 | ENSG00000187239 |
| LAMC1 | ENSG00000135862 | NSUN6 | ENSG00000241058 |
| EDEM3 | ENSG00000116406 | THNSL1 | ENSG00000185875 |
| TRMT1L | ENSG00000121486 | SVIL | ENSG00000197321 |
| MAPKAPK2 | ENSG00000162889 | CCNY | ENSG00000108100 |
| GPATCH2 | ENSG00000092978 | ZNF33A | ENSG00000189180 |
| RRP15 | ENSG00000067533 | TFAM | ENSG00000108064 |
| RAB3GAP2 | ENSG00000118873 | ANXA11 | ENSG00000122359 |
| DISP1 | ENSG00000154309 | IDE | ENSG00000119912 |
| CAPN2 | ENSG00000162909 | PGAM1 | ENSG00000171314 |
| ARF1 | ENSG00000143761 | LINC00867 | ENSG00000232139 |
| FTH1P2 | ENSG00000234975 | C10orf88 | ENSG00000119965 |
| C1orf198 | ENSG00000119280 | ACADSB | ENSG00000196177 |
| ARV1 | ENSG00000173409 | EEF1AKMT2 | ENSG00000203791 |
| LYST | ENSG00000143669 | GLRX3 | ENSG00000108010 |
| CEP170 | ENSG00000143702 | RIC8A | ENSG00000177963 |
| SMC6 | ENSG00000163029 | NAP1L4 | ENSG00000205531 |
| KIF3C | ENSG00000084731 | AC131971.1 | ENSG00000183562 |
| KHK | ENSG00000138030 | TRPC2 | ENSG00000182048 |
| GALM | ENSG00000143891 | STIM1 | ENSG00000167323 |
| THUMPD2 | ENSG00000138050 | SMPD1 | ENSG00000166311 |
| FOXN2 | ENSG00000170802 | FAR1 | ENSG00000197601 |
| RAB1A | ENSG00000138069 | PRMT3 | ENSG00000185238 |
| SFXN5 | ENSG00000144040 | BSCL2 | ENSG00000168000 |
| FUNDC2P2 | ENSG00000182814 | SLC3A2 | ENSG00000168003 |
| USP39 | ENSG00000168883 | CAPN1 | ENSG00000014216 |
| C2orf68 | ENSG00000168887 | FAM76B | ENSG00000077458 |
| KDM3A | ENSG00000115548 | CCDC82 | ENSG00000149231 |
| UXS1 | ENSG00000115652 | JRKL | ENSG00000183340 |
| CKAP2L | ENSG00000169607 | BIRC3 | ENSG00000023445 |
| INSIG2 | ENSG00000125629 | AASDHPPT | ENSG00000149313 |
| NMI | ENSG00000123609 | NPAT | ENSG00000149308 |
| STAM2 | ENSG00000115145 | ATF4P4 | ENSG00000256167 |
| TANK | ENSG00000136560 | RBM7 | ENSG00000076053 |
| COBLL1 | ENSG00000082438 | DDX6 | ENSG00000110367 |
| SPC25 | ENSG00000152253 | HINFP | ENSG00000172273 |
| NFE2L2 | ENSG00000116044 | ARHGEF12 | ENSG00000196914 |
| FTH1P20 | ENSG00000226564 | RAD51AP1 | ENSG00000111247 |
| ZNF804A | ENSG00000170396 | DYRK4 | ENSG00000010219 |
| HIBCH | ENSG00000198130 | ING4 | ENSG00000111653 |
| COQ10B | ENSG00000115520 | CD69 | ENSG00000110848 |
| SGO2 | ENSG00000163535 | KLRA1P | ENSG00000256667 |
| CLK1 | ENSG00000013441 | SMIM10L1 | ENSG00000256537 |
| PPIL3 | ENSG00000240344 | PTMAP9 | ENSG00000198134 |
| CREB1 | ENSG00000118260 | ATF7IP | ENSG00000171681 |
| KANSL1L | ENSG00000144445 | ARHGDIB | ENSG00000111348 |
| DNAJB2 | ENSG00000135924 | ALG10 | ENSG00000139133 |
| AC062015.1 | ENSG00000235070 | PPHLN1 | ENSG00000134283 |
| DIS3L2 | ENSG00000144535 | METTL7A | ENSG00000185432 |
| SH3BP4 | ENSG00000130147 | CALCOCO1 | ENSG00000012822 |
| NR1D2 | ENSG00000174738 | RPL41 | ENSG00000229117 |
| POMGNT2 | ENSG00000144647 | MYL6 | ENSG00000092841 |
| TCAIM | ENSG00000179152 | USP15 | ENSG00000135655 |
| NCKIPSD | ENSG00000213672 | C12orf29 | ENSG00000133641 |
| KLHDC8B | ENSG00000185909 | ARL1 | ENSG00000120805 |
| DAG1 | ENSG00000173402 | UBE3B | ENSG00000151148 |
| BAP1 | ENSG00000163930 | COQ5 | ENSG00000110871 |
| STIMATE | ENSG00000213533 | ATP6V0A2 | ENSG00000185344 |
| ATXN7 | ENSG00000285258 | FTH1P7 | ENSG00000232187 |
| C3orf38 | ENSG00000179021 | PDS5B | ENSG00000083642 |
| TOMM70 | ENSG00000154174 | UFM1 | ENSG00000120686 |
| ZBTB11 | ENSG00000066422 | ELF1 | ENSG00000120690 |
| ALCAM | ENSG00000170017 | RGCC | ENSG00000102760 |
| IFT57 | ENSG00000114446 | RB1 | ENSG00000139687 |
| GTPBP8 | ENSG00000163607 | SUGT1 | ENSG00000165416 |
| IQCB1 | ENSG00000173226 | TGDS | ENSG00000088451 |
| SEC22A | ENSG00000121542 | MBNL2 | ENSG00000139793 |
| MYLK | ENSG00000065534 | TEX30 | ENSG00000151287 |
| ITGB5 | ENSG00000082781 | TMCO3 | ENSG00000150403 |
| SPSB4 | ENSG00000175093 | AL589743.1 | ENSG00000244306 |
| XRN1 | ENSG00000114127 | RAB2B | ENSG00000129472 |
| U2SURP | ENSG00000163714 | SLC22A17 | ENSG00000092096 |
| PLCH1 | ENSG00000114805 | SCFD1 | ENSG00000092108 |
| NMD3 | ENSG00000169251 | COCH | ENSG00000100473 |
| NDUFB5 | ENSG00000136521 | ARHGAP5 | ENSG00000100852 |
| AP2M1 | ENSG00000161203 | AL133163.2 | ENSG00000258860 |
| IGF2BP2 | ENSG00000073792 | AL049828.1 | ENSG00000258526 |
| ACAP2 | ENSG00000114331 | KLHDC1 | ENSG00000197776 |
| PPP1R2 | ENSG00000184203 | LINC02284 | ENSG00000259719 |
| NCBP2 | ENSG00000114503 | KIAA0586 | ENSG00000100578 |
| GRK4 | ENSG00000125388 | RTN1 | ENSG00000139970 |
| WDR1 | ENSG00000071127 | ZBTB1 | ENSG00000126804 |
| DCAF16 | ENSG00000163257 | HSPA2 | ENSG00000126803 |
| LCORL | ENSG00000178177 | CCDC88C | ENSG00000015133 |
| PI4K2B | ENSG00000038210 | ATG2B | ENSG00000066739 |
| DCUN1D4 | ENSG00000109184 | VRK1 | ENSG00000100749 |
| RCHY1 | ENSG00000163743 | EVL | ENSG00000196405 |
| FAM13A | ENSG00000138640 | NBEAP1 | ENSG00000258590 |
| SMARCAD1 | ENSG00000163104 | SNRPN | ENSG00000128739 |
| EMCN | ENSG00000164035 | CCDC9B | ENSG00000188549 |
| LINC01218 | ENSG00000251636 | MAP1A | ENSG00000166963 |
| BANK1 | ENSG00000153064 | COPS2 | ENSG00000166200 |
| CXXC4 | ENSG00000168772 | DTWD1 | ENSG00000104047 |
| AC004053.1 | ENSG00000248242 | GABPB1 | ENSG00000104064 |
| AC096577.1 | ENSG00000248373 | VPS13C | ENSG00000129003 |
| HSBP1P2 | ENSG00000166530 | DENND4A | ENSG00000174485 |
| SPRY1 | ENSG00000164056 | PIAS1 | ENSG00000033800 |
| ABHD18 | ENSG00000164074 | FURIN | ENSG00000140564 |
| ELF2 | ENSG00000109381 | PCSK6 | ENSG00000140479 |
| MGST2 | ENSG00000085871 | GSPT1 | ENSG00000103342 |
| ARHGAP10 | ENSG00000071205 | NOMO2 | ENSG00000185164 |
| AC106865.1 | ENSG00000250771 | AC120114.1 | ENSG00000247735 |
| CDKN2AIP | ENSG00000168564 | VKORC1 | ENSG00000167397 |
| FRG1 | ENSG00000109536 | CA7 | ENSG00000168748 |
| AC112200.2 | ENSG00000280017 | NUTF2 | ENSG00000102898 |
| FTH1P10 | ENSG00000223361 | CDYL2 | ENSG00000166446 |
| ZFR | ENSG00000056097 | AC092718.4 | ENSG00000261061 |
| LMBRD2 | ENSG00000164187 | PAFAH1B1 | ENSG00000007168 |
| C5orf51 | ENSG00000205765 | TAX1BP3 | ENSG00000213977 |
| PAIP1 | ENSG00000172239 | P2RX1 | ENSG00000108405 |
| MTREX | ENSG00000039123 | ZBTB4 | ENSG00000174282 |
| GPBP1 | ENSG00000062194 | TMEM11 | ENSG00000178307 |
| AC016642.1 | ENSG00000248475 | PIGS | ENSG00000087111 |
| SMIM15 | ENSG00000188725 | CRLF3 | ENSG00000176390 |
| SGTB | ENSG00000197860 | SLFN14 | ENSG00000236320 |
| PIK3R1 | ENSG00000145675 | AP2B1 | ENSG00000006125 |
| AK6 | ENSG00000085231 | CCL18 | ENSG00000275385 |
| FUNDC2P1 | ENSG00000255883 | AC243829.1 | ENSG00000274767 |
| ANKRA2 | ENSG00000164331 | ATXN7L3 | ENSG00000087152 |
| SCAMP1 | ENSG00000085365 | UBE2Z | ENSG00000159202 |
| RASA1 | ENSG00000145715 | PRKCA | ENSG00000154229 |
| LYSMD3 | ENSG00000176018 | C17orf58 | ENSG00000186665 |
| RIOK2 | ENSG00000058729 | MYL6P5 | ENSG00000228118 |
| TNFAIP8 | ENSG00000145779 | KCTD2 | ENSG00000180901 |
| CEP120 | ENSG00000168944 | RPTOR | ENSG00000141564 |
| KIF3A | ENSG00000131437 | COLEC12 | ENSG00000158270 |
| AFF4 | ENSG00000072364 | IMPA2 | ENSG00000141401 |
| SAR1B | ENSG00000152700 | IMPACT | ENSG00000154059 |
| SMAD5 | ENSG00000113658 | RNF138 | ENSG00000134758 |
| SMIM3 | ENSG00000256235 | HAUS1 | ENSG00000152240 |
| SPARC | ENSG00000113140 | IER3IP1 | ENSG00000134049 |
| MFAP3 | ENSG00000037749 | ELAC1 | ENSG00000141642 |
| SLU7 | ENSG00000164609 | MED16 | ENSG00000175221 |
| AC106795.2 | ENSG00000249684 | ABHD17A | ENSG00000129968 |
| AL139095.4 | ENSG00000238221 | GIPC3 | ENSG00000179855 |
| SLC35B3 | ENSG00000124786 | PLIN3 | ENSG00000105355 |
| ASS1P1 | ENSG00000220517 | MCEMP1 | ENSG00000183019 |
| HLA-G | ENSG00000204632 | TRAPPC5 | ENSG00000181029 |
| HLA-J | ENSG00000204622 | CDC37 | ENSG00000105401 |
| MICA | ENSG00000204520 | ZNF44 | ENSG00000197857 |
| UHRF1BP1 | ENSG00000065060 | AC006213.2 | ENSG00000267058 |
| ZNF451 | ENSG00000112200 | ERCC1 | ENSG00000012061 |
| BAG2 | ENSG00000112208 | PLA2G4C | ENSG00000105499 |
| KHDRBS2 | ENSG00000112232 | FTL | ENSG00000087086 |
| PHF3 | ENSG00000118482 | ZNF347 | ENSG00000197937 |
| OGFRL1 | ENSG00000119900 | ZNF460-AS1 | ENSG00000267871 |
| SNHG5 | ENSG00000203875 | ZNF134 | ENSG00000213762 |
| WASF1 | ENSG00000112290 | C20orf96 | ENSG00000196476 |
| HDAC2-AS2 | ENSG00000228624 | TBC1D20 | ENSG00000125875 |
| VNN1 | ENSG00000112299 | RNF24 | ENSG00000101236 |
| PEX3 | ENSG00000034693 | FTLP3 | ENSG00000226608 |
| UTRN | ENSG00000152818 | CTSA | ENSG00000064601 |
| PPIL4 | ENSG00000131013 | LSM14B | ENSG00000149657 |
| GINM1 | ENSG00000055211 | BTG3 | ENSG00000154640 |
| IPCEF1 | ENSG00000074706 | C21orf91 | ENSG00000154642 |
| SFT2D1 | ENSG00000198818 | CHODL-AS1 | ENSG00000231755 |
| EIF2AK1 | ENSG00000086232 | MEMO1P1 | ENSG00000226054 |
| TMEM106B | ENSG00000106460 | MORC3 | ENSG00000159256 |
| TSPAN13 | ENSG00000106537 | PI4KA | ENSG00000241973 |
| AHR | ENSG00000106546 | BCR | ENSG00000186716 |
| OSBPL3 | ENSG00000070882 | GUSBP11 | ENSG00000228315 |
| TAX1BP1 | ENSG00000106052 | MYO18B | ENSG00000133454 |
| KBTBD2 | ENSG00000170852 | TPST2 | ENSG00000128294 |
| RP9 | ENSG00000164610 | CRYBA4 | ENSG00000196431 |
| GUSB | ENSG00000169919 | AP1B1 | ENSG00000100280 |
| KIAA1324L | ENSG00000164659 | SEC14L2 | ENSG00000100003 |
| CROT | ENSG00000005469 | ATF4 | ENSG00000128272 |
| KRIT1 | ENSG00000001631 | ENTHD1 | ENSG00000176177 |
| SEM1 | ENSG00000127922 | PACSIN2 | ENSG00000100266 |
| ARPC1B | ENSG00000130429 | BX537318.1 | ENSG00000273145 |
| CUX1 | ENSG00000257923 | TBC1D22A | ENSG00000054611 |
| ORAI2 | ENSG00000160991 | WWC3 | ENSG00000047644 |
| FBXL13 | ENSG00000161040 | TCEANC | ENSG00000176896 |
| ORC5 | ENSG00000164815 | FTLP2 | ENSG00000232368 |
| BMT2 | ENSG00000164603 | WDR13 | ENSG00000101940 |
| IQUB | ENSG00000164675 | SPIN4 | ENSG00000186767 |
| POT1 | ENSG00000128513 | P2RY10 | ENSG00000078589 |
| PTMAP10 | ENSG00000213237 | APOOL | ENSG00000155008 |
| TRBV4-1 | ENSG00000211710 | MORF4L2 | ENSG00000123562 |
| AC093673.1 | ENSG00000232533 | PSMD10 | ENSG00000101843 |
| CUL1 | ENSG00000055130 | TMEM164 | ENSG00000157600 |
| ACTR3C | ENSG00000106526 | XIAP | ENSG00000101966 |
| CNOT7 | ENSG00000198791 | PHF6 | ENSG00000156531 |
| CCDC25 | ENSG00000147419 | RAC1P4 | ENSG00000237584 |
| DCTN6 | ENSG00000104671 | MECP2 | ENSG00000169057 |
| TTI2 | ENSG00000129696 | G6PD | ENSG00000160211 |
| BAG4 | ENSG00000156735 | FUNDC2 | ENSG00000165775 |
| GPAT4 | ENSG00000158669 | DDX3Y | ENSG00000067048 |
| RB1CC1 | ENSG00000023287 | AC006378.2 | ENSG00000236861 |
| MRPL15 | ENSG00000137547 | AC087481.3 | ENSG00000270015 |
| ARFGEF1 | ENSG00000066777 | AL136382.1 | ENSG00000285210 |
| LACTB2 | ENSG00000147592 | C6orf106 | ENSG00000196821 |
| IL7 | ENSG00000104432 | H3F3B | ENSG00000132475 |
| FTH1P11 | ENSG00000237264 | H3F3C | ENSG00000270575 |
| IMPA1 | ENSG00000133731 | HIST1H3C | ENSG00000287080 |
| RBM12B | ENSG00000183808 | LINC02001 | ENSG00000267321 |
| ZNF706 | ENSG00000120963 | SEPT7P7 | ENSG00000229897 |
| KLF10 | ENSG00000155090 | | |

In some embodiments, any of the methods disclosed herein comprise determining the expression level of any additional genes listed in Table 9. In some embodiments, any of the methods disclosed herein include determining the expression level of at least 20, at least 50, at least 100, at least 200, or at least 300 additional genes selected from the genes listed in Table 9. In some embodiments, any of the methods disclosed herein include determining the expression level of 0-50, 50-100, 100-150, 150-200, 250-300 or more additional genes selected from the genes listed in Table 9. In some embodiments, any of the methods disclosed herein include determining the expression level of all 451 genes listed in Table 9. In some embodiments, the genes are weighted according to the values in Table 10.

In some embodiments, the expression level is determined by measuring mRNA level or the transcript level. In some embodiments, the mRNA level can be determined using any methods known in the art. As a non-limiting example, the mRNA level is determined using RNA-seq, real-time polymerase chain reaction (RT-PCR), Northern blot analysis, or a Ribonuclease protection assay.

In some embodiments, the platelet hyperreactivity is measured as >60% aggregation in response to submaximal epinephrine (0.4 μM).

In some embodiments, the subject has not been previously diagnosed with a cardiovascular disease. In some embodiments, the subject has been previously diagnosed with a cardiovascular disease.

In some embodiments, the condition associated with platelet hyperreactivity can be cardiovascular disease, systemic lupus erythematosus (SLE), and/or COVID-19. In some embodiments, the cardiovascular disease is atherosclerosis, peripheral artery disease (lower extremity atherosclerosis), or coronary artery disease.

In some embodiments, the cardiovascular event is death, myocardial infarction (MI), and/or stroke.

In some embodiments, the limb event is major amputation, acute limb ischemia, or a reintervention of the index limb. In some embodiments, the reintervention of the index limb comprises a new bypass graft, a jump/interposition graft revision, thrombectomy/thrombolysis, balloon angioplasty, atherectomy, laser treatment and/or stenting or stent/grafting.

In some embodiments, the antiplatelet treatment comprises one or more of aspirin, acadesine, prasugrel, ticagrelor, enilogrel, eptifibatide, cangrelor, anagrelide, anipamil, argatroban, clopidogrel, FR-122047, danaparoid sodium, dazoxiben hydrochloride, diadenosine 5′,5′″-P1,P4-tetraphosphate (Ap4A) analogs, defibrotide, dilazep dihydrochloride, 1,2- and 1,3-glyceryl dinitrate, dipyridamole, dopamine and 3-methoxytyramine, efegatran sulfate, enoxaparin sodium, glucagon, Ro-43-8857, L-700,462, ifetroban, ifetroban sodium, iloprost, isocarbacyclin methyl ester, isosorbide-5-mononitrate, itazigrel, ketanserin, BM-13.177, lamifiban, lifarizine, molsidomine, nifedipine, oxagrelate, prostaglandin E (PGE), lexipafant, prostacyclin (PGI2), pyrazines, pyridinol carbamate, abciximab, sulfinpyrazone, BN-50727, BN-52021, CV-4151, E-5510, FK-409, GU-7, KB-2796, KBT-3022, KC-404, KF-4939, OP-41483-, TRK-100, TA-3090, TFC-612 and ZK-36374, 2,4,5,7-tetrathiaoctane, 2,4,5,7-tetrathiaoctane 2,2-dioxide, 2,4,5-trithiahexane, theophylline, pentoxifylline, picotamide, sulotroban, tirofiban, trapidil, trifenagrel, trilinolein, dipyridamole, clofibrate, caffeine, ticlopidine, or an analog or derivative thereof.

In some embodiments, the subject is human.

In some embodiments, the methods can include, obtaining a sample from the subject. In one embodiment, the sample is a blood sample. In some embodiments, the blood sample comprises isolated platelets.

In some embodiments, the present disclosure provides a non-transitory computer-readable medium configured to communicate with one or more processor(s) of a computing device. In some embodiments, the non-transitory computer-readable medium includes instructions thereon that, when executed by the processor(s), cause the computing device to perform any of the methods disclosed herein.

The present disclosure also provides a computing system comprising: a memory; one or more processors; and program instructions stored in the memory that, when executed by the one or more processors, cause the computing system to (a) determine, in a blood sample collected from a subject, expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NRID2, PAPPA, PIP5K1B, PTMAP10, RAB3GAP2, RACIP4, RAD51AP1, RASAI, RTN1, SEC22A, SEM1, SEPT7P7, SLC3A2, SPARC, SPIN4, SUGTIPI, SVIL, TCAIM, THNSLI, THUMPD2, TRBV4-1, TSPAN13, UBE4B, VNN1, ZBTB11, ZFR, ZHX1-C8orf76, ZNF460-AS1, and ZNF804A; and (b) calculate a Platelet Reactivity Expression Score (PRESS) for the blood sample as a z-scored predictive probability based on weighted expression values calculated from the expression level of the genes determined in process step (a).

In some embodiments, the computing systems described herein, further comprise instructions stored in the memory that, when executed by the one or more processors, cause the computing system to determine that: (i) the subject has platelet hyperreactivity when the score is higher than a median PRESS score of a control cohort; or (ii) the subject does not have platelet hyperreactivity when the score is lower than or equal to the median PRESS score of a control cohort.

In some embodiments, the computing systems described herein, further comprise instructions stored in the memory that, when executed by the one or more processors, cause the computing system to determine that: (i) the subject is at risk of developing a cardiovascular and/or limb event when the score is higher than a median PRESS score of a control cohort; or (ii) the subject is not at risk of developing a cardiovascular and/or limb event when the score is lower than or equal to the median PRESS score of a control cohort.

In some embodiments, the computing systems described herein, further comprise instructions stored in the memory that, when executed by the one or more processors, cause the computing system to determine that: (i) the subject has a condition associated with platelet hyperreactivity when the score is higher than a median PRESS score of a control cohort; or (ii) the subject does not have a condition associated with platelet hyperreactivity when the score is lower than or equal to the median PRESS score of a control cohort.

In some embodiments, the computing systems described herein, further comprise an input/output (I/O) device; and instructions stored in the memory that, when executed by the one or more processors, cause the computing system to: (a) output, for display on the I/O device, a recommendation to administer, to the subject, one or more antiplatelet treatments when the score is higher than a median PRESS score of a control cohort.

In some embodiments, the present disclosure provides computing systems comprising: a memory; one or more processors; and program instructions stored in the memory that, when executed by the one or more processors, cause the computing system to calculate a Platelet Reactivity Expression Score (PRESS) for a blood sample collected from a subject. In some embodiments, the PRESS is calculated as a z-scored predictive probability based on weighted expression values calculated from the expression level of the following genes in the blood sample: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RACIP4, RAD51AP1, RASAI, RTN1, SEC22A, SEM1, SEPT7P7, SLC3A2, SPARC, SPIN4, SUGTIPI, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A.

In some embodiments, the computing systems described herein, further comprise instructions stored in the memory that, when executed by the one or more processors, cause the computing system to determine that: (i) the subject has platelet hyperreactivity when the score is higher than a median PRESS score of a control cohort; or (ii) the subject does not have platelet hyperreactivity when the score is lower than or equal to the median PRESS score of a control cohort.

In some embodiments, the computing systems described herein, further comprise instructions stored in the memory that, when executed by the one or more processors, cause the computing system to determine that: (i) the subject is at risk of developing a cardiovascular event when the score is higher than a median PRESS score of a control cohort; or (ii) the subject is not at risk of developing a cardiovascular event when the score is lower than or equal to the median PRESS score of a control cohort.

In some embodiments, the computing systems described herein, further comprise instructions stored in the memory that, when executed by the one or more processors, cause the computing system to determine that: (i) the subject has a condition associated with platelet hyperreactivity when the score is higher than a median PRESS score of a control cohort; or (ii) the subject does not have a condition associated with platelet hyperreactivity when the score is lower than or equal to the median PRESS score of a control cohort.

In some embodiments, the computing systems described herein, further comprise an input/output (I/O) device; and instructions stored in the memory that, when executed by the one or more processors, cause the computing system to: (a) output, for display on the I/O device, a recommendation to administer, to the subject, one or more antiplatelet treatments when the score is higher than a median PRESS score of a control cohort.

In some embodiments, the present disclosure provides a computing system for monitoring the progression of a condition associated with platelet hyperreactivity in a subject diagnosed with said condition, comprising: a memory; one or more processors; and program instructions stored in the memory that, when executed by the one or more processors, cause the computing system to: (a) determine in two or more blood samples collected from the subject at spaced apart time points expression level of the following genes AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NRID2, PAPPA, PIP5K1B, PTMAP10, RAB3GAP2, RACIP4, RAD51AP1, RASAI, RTN1, SEC22A, SEM1, SEPT7P7, SLC3A2, SPARC, SPIN4, SUGTIPI, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A; (b) calculate a Platelet Reactivity Expression Score (PRESS) for each sample as a z-scored predictive probability based on weighted expression values of the genes calculated from the expression level determined in process step (a); and (c) compare the scores calculated in process step (b) between earlier and later collected samples.

In some embodiments, the computing systems described herein, further comprise instructions stored in the memory that, when executed by the one or more processors, cause the computing system to determine that: (i) the condition in the subject has progressed when the score is increased in the later collected sample(s) as compared to the earlier collected sample(s), or (ii) the condition in the subject has not progressed when the score is not increased in the later collected sample(s) as compared to the earlier collected sample(s).

In some embodiments, the present disclosure provides computing systems for determining the effect of an antiplatelet treatment on development of a condition associated with platelet hyperreactivity in a subject diagnosed with said condition, comprising: a memory; one or more processors; and program instructions stored in the memory that, when executed by the one or more processors, cause the computing system to: (a) determine, in blood samples collected from the subject prior to and after administering the antiplatelet treatment, expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIPSK1B, PTMAP10, RAB3GAP2, RAC1P4, RAD51AP1, RASAI, RTN1, SEC22A, SEM1, SEPT7P7, SLC3A2, SPARC, SPIN4, SUGTIP1, SVIL, TCAIM, THNSLI, THUMPD2, TRBV4-1, TSPAN13, UBE4B, VNN1, ZBTB11, ZFR, ZHX1-C8orf76, ZNF460-AS1, and ZNF804A; (b) calculate a Platelet Reactivity Expression Score (PRESS) for each sample as a z-scored predictive probability based on weighted expression values calculated from the expression level of the genes determined in process step (a).

In some embodiments, the computing systems described herein, further comprise instructions stored in the memory that, when executed by the one or more processors, cause the computing system to compare the scores calculated in process step (b) between samples collected from the subject prior to and after administering the antiplatelet treatment, determine that (i) the antiplatelet treatment is effective when the score is decreased in the sample(s) collected after administering the antiplatelet treatment as compared to the sample(s) collected before administering the antiplatelet treatment, or (ii) the antiplatelet treatment is not effective when the score is the same or increased in the sample(s) collected after administering the antiplatelet treatment as compared to the sample(s) collected before administering the antiplatelet treatment.

In some embodiments, the present disclosure provides computing systems for identifying an antiplatelet treatment useful for slowing down the progression or treating a condition associated with platelet hyperreactivity in a subject diagnosed with said condition, comprising: a memory; one or more processors; and program instructions stored in the memory that, when executed by the one or more processors, cause the computing system to: (a) determine in blood samples collected from the subject prior to and after administering a test antiplatelet treatment expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NRID2, PAPPA, PIP5K1B, PTMAP10, RAB3GAP2, RACIP4, RAD51AP1, RASAI, RTN1, SEC22A, SEM1, SEPT7P7, SLC3A2, SPARC, SPIN4, SUGTIPI, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A; and (b) calculate a Platelet Reactivity Expression Score (PRESS) for each sample as a z-scored predictive probability based on weighted expression values calculated from the expression level of the genes determined in process step (a).

In some embodiments, the computing systems described herein, further comprise instructions stored in the memory that, when executed by the one or more processors, cause the computing system to compare the scores calculated in process step (b) between samples collected from the subject prior to and after administering the test antiplatelet treatment; and determine that (i) the test antiplatelet treatment is effective for slowing down the progression or treating the condition when the score is decreased in the sample(s) collected after administering the test antiplatelet treatment as compared to the sample(s) collected before administering the test antiplatelet treatment, or (ii) the test antiplatelet treatment is not effective for slowing down the progression or treating the condition when the score is the same or increased in the sample(s) collected after administering the test antiplatelet treatment as compared to the sample(s) collected before administering the test antiplatelet treatment.

Also provided herein is a computing system comprising: a memory; one or more processors; and program instructions stored in the memory that, when executed by the one or more processors, cause the computing system to: calculate a first value of a subject as a z-scored predictive probability based on weighted expression values calculated from an expression level of a subset of genes; and output a recommendation to administer, to the subject, one or more antiplatelet treatments when the value is higher than a median value score of a control cohort, wherein the expression level is determined for the following genes in a blood sample of the subject: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASAI, RTN1, SEC22A, SEM1, SEPT7P7, SLC3A2, SPARC, SPIN4, SUGTIPI, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A. In some embodiments, the first value is a Platelet Reactivity Expression Score (PRESS).

FIG. 11 illustrates a block diagram of an embodiment of a computing device 1100. As shown, computing device 1100 may include one or more processor(s) 1110, an input/output (I/O) device 1120, a memory 1130 containing an operating system (“OS”) 1140, a database 1150, and a program 1160. In some embodiments, instructions are stored in the memory 1130 that are executable by the processor 1110 to perform steps of the computational methods disclosed herein. The computing device can include one or more modules or engines for carrying out computational methods disclosed herein. In some embodiments, the I/O device 1120 is configured to communicate with the respective ancillary features such as databased or software computational tools to carry out the functions and computational steps disclosed herein.

Computing device 1100 may be a single server or may be configured as a distributed computer system including multiple servers or computers that interoperate to perform one or more of the processes and functionalities associated with the disclosed embodiments. In some embodiments, computing device 1100 may further include a peripheral interface, a transceiver, a mobile network interface in communication with processor 1110, a bus configured to facilitate communication between the various components of computing device 1100, and a power source configured to power one or more components of computing device 1100. A peripheral interface may include the hardware, firmware and/or software that enables communication with various peripheral devices, such as media drives (e.g., magnetic disk, solid state, or optical disk drives), other processing devices, or any other input source used in connection with the instant techniques. In some embodiments, a peripheral interface may include a serial port, a parallel port, a general-purpose input and output (GPIO) port, a game port, a universal serial bus (USB), a micro-USB port, a high-definition multimedia (HDMI) port, a video port, an audio port, a Bluetooth™ port, an NFC port, another like communication interface, or any combination thereof.

In some embodiments, a transceiver may be configured to communicate with compatible devices and ID tags when they are within a predetermined range. A transceiver may be compatible with one or more of: RFID, NFC, Bluetooth™, low-energy Bluetooth™ (BLE), WiFi™, ZigBee™, ABC protocols or similar technologies.

A mobile network interface may provide access to a cellular network, the Internet, or another wide-area network. In some embodiments, a mobile network interface may include hardware, firmware, and/or software that allows processor 1110 to communicate with other devices via wired or wireless networks, whether local or wide area, private or public, as known in the art. A power source may be configured to provide an appropriate alternating current (AC) or direct current (DC) to power components.

Processor 1110 may include one or more of a microprocessor, microcontroller, digital signal processor, co-processor or the like or combinations thereof capable of executing stored instructions and operating upon stored data. Memory 1130 may include, in some implementations, one or more suitable types of memory (e.g., volatile or non-volatile memory, random access memory (RAM), read only memory (ROM), programmable read-only memory (PROM), erasable programmable read-only memory (EPROM), electrically erasable programmable read-only memory (EEPROM), magnetic disks, optical disks, floppy disks, hard disks, removable cartridges, flash memory, a redundant array of independent disks (RAID), and the like) for storing files, including an operating system, application programs (including, e.g., a web browser application, a widget or gadget engine, or other applications, as necessary), executable instructions, and data. In one embodiment, the processing techniques described herein are implemented as a combination of executable instructions and data within memory 1130.

Processor 1110 may be one or more known processing devices, such as a microprocessor from the Pentium™ family manufactured by Intel™ or the Turion™ family manufactured by AMD™. Processor 1110 may constitute a single core or multiple core processor that executes parallel processes simultaneously. For example, processor 1110 may be a single core processor that is configured with virtual processing technologies. In certain embodiments, processor 1110 may use logical processors to simultaneously execute and control multiple processes. Processor 1110 may implement virtual machine technologies, or other similar known technologies to provide the ability to execute, control, run, manipulate, store, etc. multiple software processes, applications, programs, etc. Other types of processor arrangements could be implemented that provide for the capabilities disclosed herein as understood by a person skilled in the pertinent art.

Computing device 1100 may include one or more storage devices configured to store information used by processor 1110 (or other components) to perform certain functions related to the disclosed embodiments. In one example, computing device 1100 may include memory 1130 that includes instructions to enable processor 1110 to execute one or more applications, such as server applications, network communication processes, and any other type of application or software known to be available on computer systems. Alternatively, the instructions, application programs, etc., may be stored in an external storage or available from a memory over a network. The one or more storage devices may be a volatile or non-volatile, magnetic, semiconductor, tape, optical, removable, non-removable, or other type of storage device or tangible computer-readable medium.

In one embodiment, computing device 1100 may include memory 1130 that includes instructions that, when executed by processor 1110, perform one or more processes consistent with the functionalities disclosed herein. Methods, systems, and articles of manufacture consistent with disclosed embodiments are not limited to separate programs or computers configured to perform dedicated tasks. For example, computing device 1100 may include memory 1130 that may include one or more programs 1160 to perform one or more functions of the disclosed embodiments. Moreover, processor 1110 may execute one or more programs 1160 located remotely from computing device 1100. For example, computing device 1100 may access one or more remote programs 1160, that, when executed, perform functions related to disclosed embodiments.

Memory 1130 may include one or more memory devices that store data and instructions used to perform one or more features of the disclosed embodiments. Memory 1130 may also include any combination of one or more databases controlled by memory controller devices (e.g., server(s), etc.) or software, such as document management systems, Microsoft™ SQL databases, SharePoint™ databases, Oracle™ databases, Sybase™ databases, or other relational databases. Memory 1130 may include software components that, when executed by processor 1110, perform one or more processes consistent with the disclosed embodiments. In some embodiments, memory 1130 may include database 1150 for storing related data to enable computing device 1100 to perform one or more of the processes and functionalities associated with the disclosed embodiments.

Computing device 1100 may also be communicatively connected to one or more memory devices (e.g., databases (not shown)) locally or through a network. The remote memory devices may be configured to store information and may be accessed and/or managed by computing device 1100. By way of example, the remote memory devices may be document management systems, Microsoft™ SQL database, SharePoint™ databases, Oracle™ databases, Sybase™ databases, or other relational databases. Systems and methods consistent with disclosed embodiments, however, are not limited to separate databases or even to the use of a database.

Computing device 1100 may also include one or more I/O devices 1120 that may include one or more interfaces for receiving signals or input from devices and providing signals or output to one or more devices that allow data to be received and/or transmitted by computing device 1100. For example, computing device 1100 may include interface components, which may provide interfaces to one or more input devices, such as one or more keyboards, mouse devices, touch screens, track pads, trackballs, scroll wheels, digital cameras, microphones, sensors, and the like, that enable computing device 1100 to receive data from one or more users. Additionally, computing device 1100 may include one or more display devices, which may provide visual output to users. A display may include any device capable of presenting visual information, such as liquid crystal displays (LCDs), light-emitting diode (LED) displays, organic LED (OLED) displays, cathode ray tube (CRT) monitors, or other types of screens or projectors. These displays may be used to convey information, graphics, video, or other visual content to users, facilitating interaction with computing device 1100.

In example embodiments of the disclosed technology, computing device 1100 may include any number of hardware and/or software applications that are executed to facilitate any of the operations. The one or more I/O interfaces may be utilized to receive or collect data and/or user instructions from a wide variety of input devices. Received data may be processed by one or more computer processors as desired in various implementations of the disclosed technology and/or stored in one or more memory devices.

While computing device 1100 has been described as one form for implementing the techniques described herein, other, functionally equivalent techniques may be employed as understood by a person skilled in the pertinent art. For example, as known in the art, some or all of the functionality implemented via executable instructions may also be implemented using firmware and/or hardware devices such as application specific integrated circuits (ASICs), programmable logic arrays, state machines, etc. Furthermore, other implementations may include a greater or lesser number of components than those illustrated.

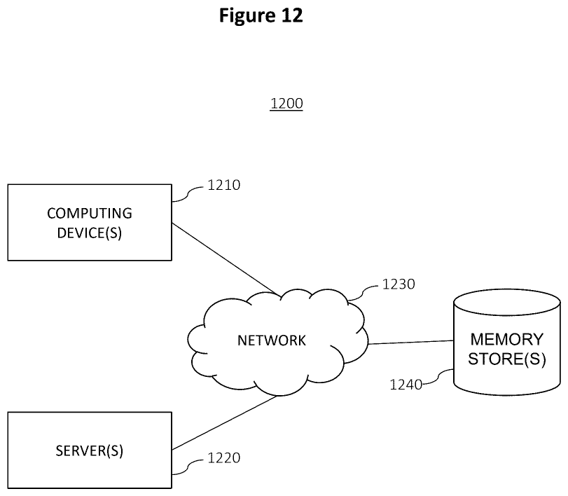

FIG. 12 illustrates a block diagram of an embodiment of a computing network 1200 including computing device(s) 1210, server(s) 1220, memory store(s) 1240, and a network 1230 facilitating communication between each. In some embodiments, the network 1200 is configured to execute steps of computational methods as disclosed herein. In some embodiments, one or more computing device(s) 1210 include various engines and/or modules as disclosed elsewhere herein, which may be distributed across the computing device(s). The computing device(s) are configured to communicate with ancillary databases and/or software services to carry out steps of computational methods disclosed herein.

Network 1230 may be of any suitable type, including individual connections via the internet such as cellular or WiFi™ networks. In some embodiments, network 1230 may connect terminals, services, and mobile devices using direct connections such as radio-frequency identification (RFID), near-field communication (NFC), Bluetooth™, low-energy Bluetooth™ (BLE), WiFi™, ZigBee™, ambient backscatter communications (ABC) protocols, USB, WAN, or LAN. Because the information transmitted may be personal or confidential, security concerns may dictate one or more of these types of connections be encrypted or otherwise secured. In some embodiments, however, the information being transmitted may be less personal, and therefore the network connections may be selected for convenience over security.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The terms “a,” “an,” and “the” do not denote a limitation of quantity, but rather denote the presence of “at least one” of the referenced item.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The terms “patient”, “individual”, “subject”, and “animal” are used interchangeably herein and refer to mammals, including, without limitation, human and veterinary animals (e.g., cats, dogs, cows, horses, goats, sheep, pigs, etc.) and experimental animal models. In one embodiment, the subject is a human.

As used herein, the term “platelet hyperreactivity” is defined as >60% aggregation of platelets in response to submaximal epinephrine (0.4 μM).

### EXAMPLES

The following examples are provided to further describe some of the embodiments disclosed herein. The examples are intended to illustrate, not to limit, the disclosed embodiments.

### Example 1. Prevalence of Major Adverse Cardiovascular and Limb Events (MACLE) in Patients after Lower Extremity Revascularization (LER) Procedure

A total of 300 patients scheduled for a LER procedure were enrolled. Of these, 13 individuals were subsequently excluded because a procedure was not performed (n=7), another exclusion was noted (n=4), or they died during index hospitalization from hemorrhagic complications (n=2, FIG. 6). Among the remaining 287 patients, open (n=118, 41.1%), endovascular (n=121, 42.2%), and hybrid (n=28, 9.8%) procedures were performed. In a small subset of patients, a peripheral angiogram was performed without revascularization (n=20, 6.9%). The mean age was 70±11 years, 33% were female, and 61% were white. More than half of all patients (54%) had known coronary artery disease, 53% had diabetes mellitus, and 78% presented with CLI (Table 2). Before surgery, the median (IQR) ankle-brachial index (ABI) was 0.54 (0.41, 0.72).

Thirty days post LER, 54 (18.8%) patients experienced a MACLE. Patients with a postoperative MACLE were more likely to present with CLI (90.7% vs. 75.5%, p=0.024, Table 2). Antiplatelet therapy, beta-blockers, statins, and ACE inhibitors/angiotensin receptor blockers were not significantly different between patients and those without post-procedural MACLE.

TABLE 2
Demographics and Clinical Characteristics of PACE-PAD Study Cohort Stratified by MACLE

| | Overall | | MACLE | | No MACLE | | P |
|---|---|---|---|---|---|---|---|
| | (n = 287) | | (n = 54) | | (n = 233) | | value |
| Age, mean (SD) | 69.5 | (10.7) | 70.3 | (9.6) | 69.4 | (11.0) | 0.576 |
| Female, n (% ) | 94 | (32.8) | 17 | (31.5) | 77 | (33.0) | 0.952 |
| Race, n (% ) | | | | | | | 0.277 |
| White | 176 | (61.3) | 29 | (53.7) | 147 | (63.1) | |
| Black | 66 | (23.0) | 13 | (24.1) | 53 | (22.7) | |
| Asian | 5 | (1.7) | 2 | (3.7) | 3 | (1.3) | |
| Other | 40 | (13.9) | 10 | (18.5) | 30 | (12.9) | |
| Ethnicity | | | | | | | |
| Hispanic | 54 | (19.0) | 14 | (25.9) | 40 | (17.4) | 0.213 |
| BMI, mean (SD) | 26.85 | (5.35) | 25.84 | (5.14) | 27.09 | (5.38) | 0.123 |
| Smoking, n (% ) | | | | | | | 0.230 |
| Current | 49 | (17.1) | 11 | (20.4) | 38 | (16.4) | |
| Former | 167 | (68.4) | 26 | (48.1) | 141 | (60.8) | |
| Never | 70 | (24.5) | 17 | (31.5) | 53 | (22.8) | |
| Family History of CVD, n | 80 | (28.5) | 9 | (17.3) | 71 | (31.0) | 0.071 |
| (%) | | | | | | | |
| Clinical History, n (%) | | | | | | | |
| CAD | 156 | (54.4) | 32 | (59.3) | 124 | (53.2) | 0.515 |
| Prior MI | 73 | (25.4) | 17 | (31.5) | 56 | (24.0) | 0.338 |
| Prior CVA | 52 | (18.1) | 14 | (25.9) | 38 | (16.3) | 0.145 |
| Carotid artery disease | 44 | (15.3) | 8 | (14.8) | 36 | (15.5) | 1 |
| VTE | 38 | (13.2) | 9 | (16.7) | 29 | (12.4) | 0.547 |
| Diabetes | 151 | (52.6) | 30 | (55.6) | 121 | (51.9) | 0.742 |
| COPD | 52 | (18.1) | 10 | (18.5) | 42 | (18.0) | 1 |
| Hypertension | 253 | (88.2) | 44 | (81.5) | 209 | (89.7) | 0.147 |
| Hyperlipidemia | 215 | (74.9) | 42 | (77.8) | 173 | (74.2) | 0.715 |
| Cancer | 63 | (22.0) | 9 | (16.7) | 54 | (23.2) | 0.39 |
| Heart Failure | 53 | (18.5) | 11 | (20.4) | 42 | (18.0) | 0.837 |
| Procedure type, n (%) | | | | | | | 0.088 |
| Open | 118 | (41.1) | 15 | (27.8) | 103 | (44.2) | |
| Endovascular | 121 | (42.4) | 25 | (46.3) | 96 | (41.2) | |
| Hybrid | 28 | (9.8) | 8 | (14.8) | 20 | (8.6) | |
| No intervention | 20 | (7.0) | 6 | (11.1) | 14 | (6.0) | |
| Indication, n (%) | | | | | | | |
| Critical Limb Ischemia | 225 | (78.4) | 49 | (90.7) | 176 | (75.5) | 0.024 |
| Rest pain | 119 | (41.5) | 23 | (42.6) | 96 | (41.2) | 0.973 |
| Gangrene | 216 | (75.3) | 18 | (33.3) | 53 | (22.7) | 0.147 |
| Ulceration | 116 | (40.4) | 24 | (44.4) | 92 | (39.5) | 0.606 |
| Lowest ABI, median (IQR) | 0.54 | [0.41, 0.73] | 0.52 | [0.37, 0.80] | 0.54 | [0.41, 0.72] | 0.728 |
| ABI in index limb, median | 0.59 | [0.43, 0.87] | 0.54 | [0.38, 1.03] | 0.59 | [0.43, 0.80] | 0.989 |
| (IQR) | | | | | | | |
| Baseline Medication, n (%) | | | | | | | |
| Antiplatelet Therapy | 256 | (89.2) | 49 | (90.7) | 207 | (88.8) | 0.871 |
| Aspirin | 234 | (81.5) | 46 | (85.2) | 188 | (80.7) | 0.567 |
| Clopidogrel | 118 | (41.1) | 21 | (38.9) | 97 | (41.6) | 0.829 |
| Statin | 234 | (81.5) | 47 | (87.0) | 187 | (80.3) | 0.336 |
| Beta Blocker | 172 | (59.9) | 35 | (64.8) | 137 | (58.8) | 0.51 |
| ACE/ARB | 156 | (54.4) | 28 | (51.9) | 128 | (54.9) | 0.796 |
| Baseline Lab Values | | | | | | | |
| Creatinine, mg/dl, median | 1.0 | [0.8, 1.4] | 1.0 | [0.8, 1.6] | 1.0 | [0.8, 1.4] | 0.90 |
| (IQR) | | | | | | | |

ABI = ankle-brachial index; ACE/ARB = angiotensin-converting enzyme/angiotensin receptor blocker; BMI = body mass index; CAD = coronary artery disease; CVA = cerebral vascular accident; CVD = cardiovascular disease; MACLE = major adverse cardiac and limb events, defined as a composite of death, myocardial infarction (MI), stroke, major amputation, or acute limb ischemia leading to revascularization; MI = myocardial infarction; VTE = venous thromboembolism; COPD = Chronic obstructive pulmonary disease; IQR = inter-quartile range.

### Example 2. Platelet Hyperreactivity is Associated with 30-Day MACLE

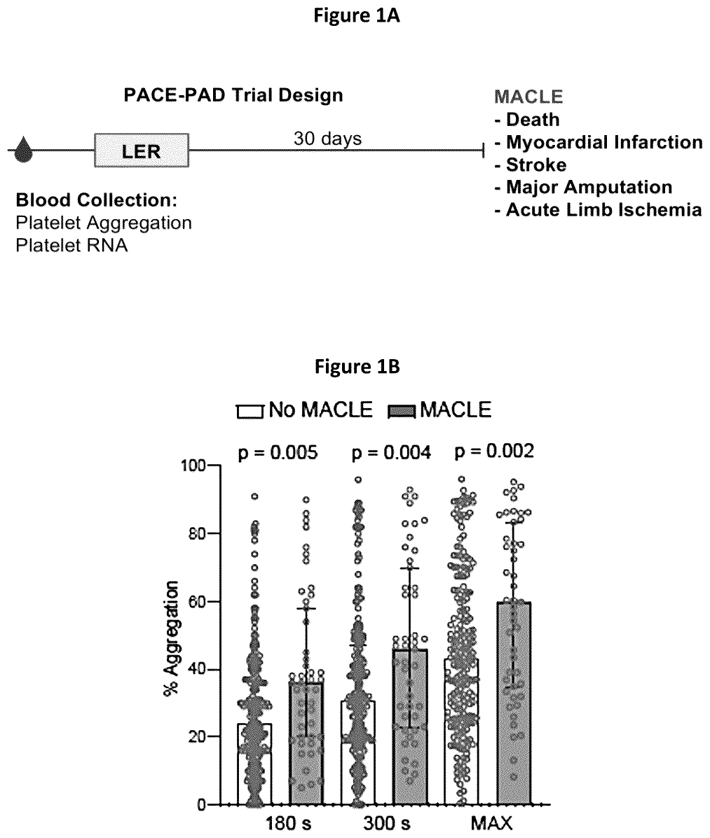

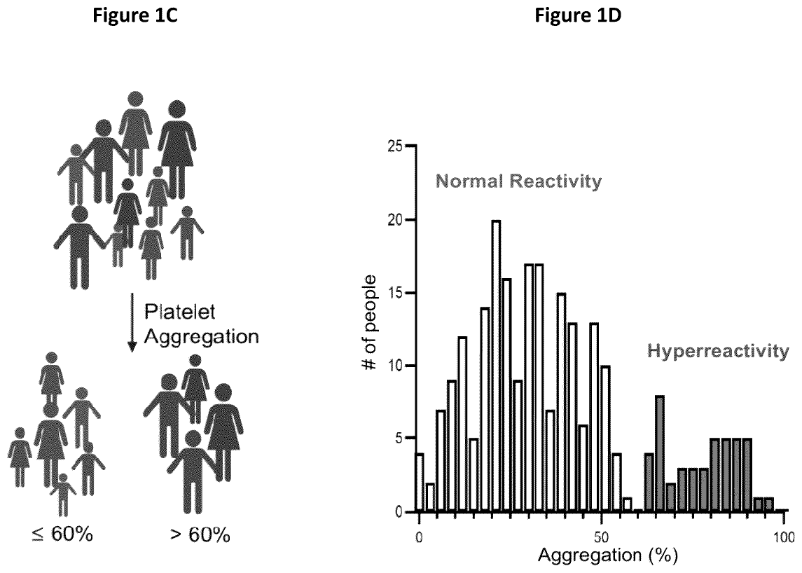

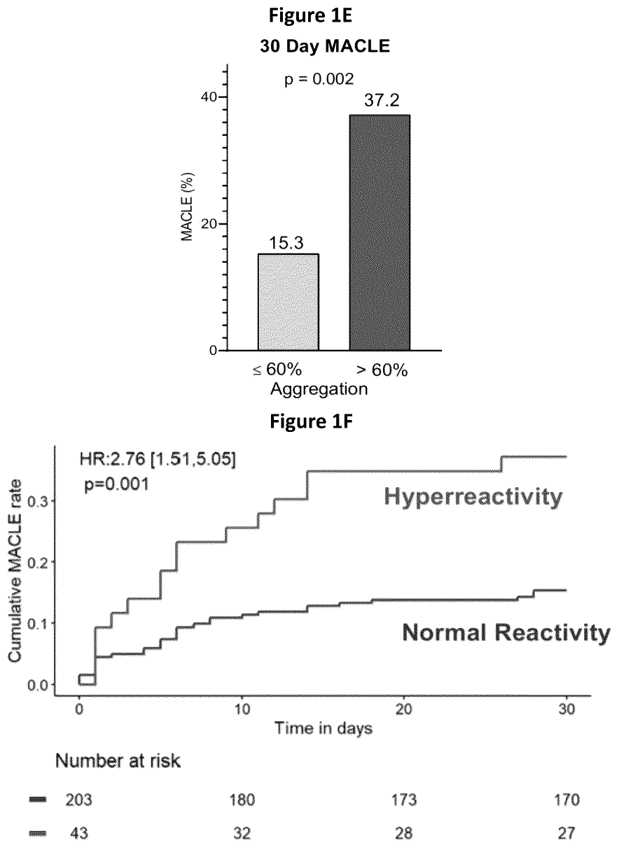

Directly before LER, platelet aggregation was measured in 254 (88.5%) patients (FIG. 1A). The baseline characteristics of patients with and without assessment of platelet aggregation are described in Table 3. Median platelet aggregation in response to 0.4 μM epinephrine was 32% (IQR 20, 49) and was significantly higher in patients with versus without MACLE (FIG. 1B). Platelet aggregation in response to epinephrine at multiple doses and time points was consistently higher in patients with MACLE (Table 4). In addition, patients with MACLE had significantly higher platelet aggregation in response to other agonists at submaximal doses (e.g., arachidonic acid (AA), ADP, collagen, serotonin) than patients who did not go on to develop a major cardiac or limb event (Table 4). These data demonstrate that platelet reactivity before LER is strongly associated with MACLE 30 days postoperatively.

TABLE 3
Demographics and clinical characteristics in PACE-PAD
patients with and without platelet aggregation measured

| | Platelet | | No Platelet | | |
|---|---|---|---|---|---|
| | Aggregation | | Aggregation | | |
| | Measured | | Measured | | P |
| | (n = 254) | | (n = 33) | | value |
| Age, mean (SD) | 69.7 | (10.71) | 68.6 | (10.94) | 0.572 |
| Female, n (%) | 79 | (31.1) | 15 | (45.5) | 0.146 |
| Race | | | | | 0.026 |
| White | 160 | (63.0) | 16 | (48.5) | |
| Black | 52 | (20.5) | 14 | (42.4) | |
| Asian | 4 | (1.6) | 1 | (3.0) | |
| Other | 38 | (15.0) | 2 | (6.1) | |
| Race | | | | | 0.156 |
| White | 160 | (63.0) | 16 | (48.5) | |
| Other | 94 | (37.0) | 17 | (51.5) | |
| Ethnicity | | | | | |
| Hispanic | 49 | (19.4) | 5 | (15.6) | 0.780 |
| BMI, mean (SD) | 26.8 | (5.31) | 27.0 | (5.76) | 0.849 |
| Smoking | | | | | 0.397 |
| Current | 43 | (17.0) | 6 | (18.2) | |
| Former | 151 | (59.7) | 16 | (48.5) | |
| Never | 59 | (23.3) | 11 | (33.3) | |
| Family History of CVD | 72 | (29.0) | 8 | (24.2) | 0.713 |
| Clinical History | | | | | |
| CAD | 137 | (53.9) | 19 | (57.6) | 0.834 |
| Prior MI | 63 | (24.8) | 10 | (30.3) | 0.638 |
| Prior CVA | 46 | (18.1) | 6 | (18.2) | 1 |
| Carotid artery disease | 37 | (14.6) | 7 | (21.2) | 0.459 |
| VTE | 33 | (13.0) | 5 | (15.2) | 0.943 |
| Diabetes | 126 | (49.6) | 25 | (75.8) | 0.008 |
| COPD | 46 | (18.1) | 6 | (18.2) | 1 |
| Hypertension | 221 | (87.0) | 32 | (97.0) | 0.148 |
| Hyperlipidemia | 190 | (74.8) | 25 | (75.8) | 1 |
| Cancer | 59 | (23.2) | 4 | (12.1) | 0.183 |
| Heart Failure | 44 | (17.3) | 9 | (27.3) | 0.251 |

ABI = ankle-brachial index; ACE/ARB = angiotensin-converting enzyme/angiotensin receptor blocker; BMI = body mass index; CAD = coronary artery disease; CVA = cerebral vascular accident; CVD = cardiovascular disease; MI = myocardial infarction; VTE = venous thromboembolism; COPD = Chronic obstructive pulmonary disease.

TABLE 4
Platelet Aggregation Stratified by MACLE

| | Overall | | MACLE | | No MACLE | | P value |
|---|---|---|---|---|---|---|---|
| Epinephrine 2 μM | | | | | | | |
| 180 s, median (25th, 75th) | 42 | [27, 58] | 51 | [37, 76] | 41 | [27, 57] | 0.003 |
| 300 s, median (25th, 75th) | 50 | [33, 69] | 63 | [45, 83] | 48 | [31, 64] | 0.001 |
| max, median (25th, 75th) | 63 | [44, 80] | 76 | [52, 86] | 60 | [42, 77] | 0.001 |
| Epinephrine 0.4 μM | | | | | | | |
| 180 s, median (25th, 75th) | 27 | [16, 40] | 36 | [20, 56] | 24 | [16, 37] | 0.005 |
| 300 s, median (25th, 75th) | 32 | [20, 49] | 46 | [25, 67] | 31 | [19, 47] | 0.004 |
| max, median (25th, 75th) | 45 | [28 68] | 60 | [35, 81] | 43 | [26, 62] | 0.002 |
| Epinephrine 0.1 μM | | | | | | | |
| 180 s, median (25th, 75th) | 12 | [7, 22] | 17 | [10, 27] | 12 | [6, 22] | 0.02 |
| 300 s, median (25th, 75th) | 19 | [10, 29] | 26 | [14, 39] | 18 | [9, 27] | 0.003 |
| max, median (25th, 75th) | 27 | [16, 43] | 37 | [20, 69] | 26 | [15, 40] | 0.005 |
| ADP 2 μM | | | | | | | |
| 180 s, median (25th, 75th) | 53 | [27, 70] | 61 | [45, 77] | 48 | [26, 69] | 0.009 |
| 300 s, median (25th, 75th) | 48 | [21, 69] | 61 | [46, 78] | 44 | [21, 67] | 0.004 |
| max, median (25th, 75th) | 59 | [41, 73] | 66 | [55, 79] | 56 | [40, 71] | 0.01 |
| ADP 1 μM | | | | | | | |
| 180 s, median (25th, 75th) | 23 | [8, 47] | 39 | [19, 63] | 20 | [5, 42] | 0.001 |
| 300 s, median (25th, 75th) | 22 | [8, 46] | 40 | [18, 67] | 20 | [7, 40] | 0.001 |
| max, median (25th, 75th) | 38 | [21, 56] | 50 | [29, 68] | 34 | [19, 53] | 0.004 |
| ADP 0.4 μM | | | | | | | |
| 180 s, median (25th, 75th) | 6 | [0, 15] | 15 | [0, 30.50] | 4 | [0, 12] | 0.001 |
| 300 s, median (25th, 75th) | 8 | [0, 18] | 14.50 | [2, 32] | 7 | [0, 15] | 0.002 |
| max, median (25th, 75th) | 16 | [8, 28] | 25 | [9, 42] | 15 | [7, 25] | 0.005 |
| Collagen 1 μg/ml | | | | | | | |
| 180 s, median (25th, 75th) | 7 | [2, 17] | 10 | [4, 22] | 7 | [2, 15.25] | 0.05 |
| 300 s, median (25th, 75th) | 11 | [4, 20] | 17 | [7, 31] | 11 | [4, 19] | 0.037 |
| max, median (25th, 75th) | 17 | [9, 29] | 24 | [11, 42] | 16 | [9, 26] | 0.011 |
| Collagen 0.2 μg/ml | | | | | | | |
| 180 s, median (25th, 75th) | 3 | [0, 8] | 6 | [2, 10] | 3 | [0, 7] | 0.007 |
| 300 s, median (25th, 75th) | 7 | [2, 12] | 9 | [4, 18] | 6 | [2, 11] | 0.01 |
| max, median (25th, 75th) | 13 | [7, 22] | 18 | [10, 31] | 11 | [7, 21] | 0.007 |
| Serotonin 10 μM | | | | | | | |
| 180 s, median (25th, 75th) | 2 | [0, 8] | 7 | [0, 14] | 0 | [0, 6] | 0.001 |
| 300 s, median (25th, 75th) | 3 | [0, 10] | 9 | [2, 16] | 3 | [0, 9] | 0.001 |
| max, median (25th, 75th) | 13 | [7, 22] | 20 | [9, 26] | 13 | [7, 21] | 0.017 |
| Serotonin 10 μM + | | | | | | | |
| Epinephrine 0.1 μM | | | | | | | |
| 180 s, median (25th, 75th) | 14 | [4, 33] | 22 | [9, 39] | 14 | [3, 30] | 0.043 |
| 300 s, median (25th, 75th) | 16 | [6, 32] | 27 | [11, 46] | 15 | [4, 30] | 0.011 |
| max, median (25th, 75th) | 29 | [17, 45] | 37 | [20, 65] | 27 | [16, 41] | 0.009 |
| Arachidonic Acid 1600 μM | | | | | | | |
| 180 s, median (25th, 75th) | 25 | [9, 62] | 34 | [17, 74] | 22 | [9, 47] | 0.009 |
| 300 s, median (25th, 75th) | 29 | [11, 69] | 40 | [19, 80] | 27 | [10, 62] | 0.01 |
| max, median (25th, 75th) | 37 | [16, 77] | 63 | [24, 84] | 33 | [15, 73] | 0.005 |
| Arachidonic Acid ex vivo | | | | | | | |
| 1600 μM | | | | | | | |
| 180 s, median (25th, 75th) | 10 | [4, 21] | 17 | [9, 24.50] | 10 | [2, 20] | 0.066 |
| 300 s, median (25th, 75th) | 13 | [5, 25] | 20 | [10, 29] | 12 | [4, 23] | 0.026 |
| max, median (25th, 75th) | 18 | [8, 32] | 22 | [14, 39] | 17 | [8, 30] | 0.023 |

MACLE = major adverse cardiac and limb events, defined as a composite of death, myocardial infarction (MI), stroke, major amputation, or acute limb ischemia leading to revascularization.

### Example 3. Platelet Hyperreactivity and Clinical Presentation

In response to submaximal epinephrine, platelet aggregation induced a bimodal response where a small yet significant proportion of individuals demonstrated a hyperreactive platelet phenotype with >60% aggregation (FIGS. 1C, 1D). Among patients with aggregation measured in response to 0.4 μM epinephrine, 17.5% displayed platelet hyperreactivity. Patients with a hyperreactive platelet phenotype did not differ by age, sex, or race/ethnicity but were more likely to have diabetes mellitus, present with gangrene, and less likely to receive antiplatelet therapy (Table 5). Following multivariable adjustment, gangrene (aOR 2.50, 95% CI 1.12 to 5.54) was associated with higher odds of platelet hyperreactivity and antiplatelet therapy (aOR 0.15, 95% CI 0.06 to 0.39) with lower odds of platelet hyperreactivity. After excluding 31 (10.7%) patients not on antiplatelet therapy, gangrene was the only covariate associated with higher odds (aOR 2.98, 95% CI 1.40 to 6.34) of platelet hyperreactivity.

TABLE 5
Demographics and clinical characteristics by platelet hyperreactivity
to 0.4 μM epinephrine (n = 246).

| | No | | | | |
|---|---|---|---|---|---|
| | Hyperreactivity ≤60% | | Hyperreactivity >60% | | |
| | Aggregation | | Aggregation | | P |
| | (n = 203) | | (n = 43) | | value |
| Age, mean (SD) | 70.04 | (10.4) | 69.79 | (10.4) | 0.887 |
| Female, n (%) | 64 | (31.5) | 12 | (27.9) | 0.776 |
| Race, n (%) | | | | | 0.084 |
| White | 134 | (66.0) | 22 | (51.2) | |
| Black | 42 | (20.7) | 10 | (23.3) | |
| Asian | 2 | (1.0) | 2 | (4.7) | |
| Other | 25 | (12.3) | 9 | (20.9) | |
| Ethnicity | | | | | |
| Hispanic | 35 | (17.3) | 11 | (25.6) | 0.297 |
| BMI, mean (SD) | 26.9 | (5.4) | 26.8 | (5.4) | 0.898 |
| Smoking, n (%) | | | | | 0.234 |
| Current | 36 | (17.8) | 5 | (11.6) | |
| Former | 123 | (60.9) | 24 | (55.8) | |
| Never | 43 | (21.3) | 14 | (32.6) | |
| Family History of CVD, n (%) | 59 | (29.6) | 11 | (26.2) | 0.794 |
| Clinical History, n (%) | | | | | |
| CAD | 113 | (55.7) | 21 | (48.8) | 0.517 |
| Prior MI | 53 | (26.1) | 8 | (18.6) | 0.401 |
| Prior CVA | 34 | (16.7) | 11 | (25.6) | 0.253 |
| Carotid artery disease | 29 | (14.3) | 7 | (16.3) | 0.922 |
| VTE | 24 | (11.8) | 8 | (18.6) | 0.341 |
| Diabetes | 96 | (47.3) | 29 | (67.4) | 0.026 |
| COPD | 42 | (20.7) | 3 | (7.0) | 0.048 |
| Hypertension | 174 | (85.7) | 41 | (95.3) | 0.126 |
| Hyperlipidemia | 148 | (72.9) | 36 | (83.7) | 0.197 |
| Cancer | 52 | (25.6) | 7 | (16.3) | 0.269 |
| Heart Failure | 35 | (17.2) | 7 | (16.3) | 1 |
| Indication | | | | | |
| Rest pain, Gangrene or Ulceration | 156 | (76.8) | 37 | (86.0) | 0.259 |
| Rest pain | 87 | (42.9) | 15 | (34.9) | 0.429 |
| Gangrene | 38 | (18.7) | 20 | (46.5) | <0.001 |
| Ulceration | 85 | (41.9) | 16 | (37.2) | 0.694 |
| Lowest ABI, median (IQR) | 0.54 | [0.41, 0.72] | 0.53 | [0.41, 0.70] | 0.689 |
| ABI in index limb, median (IQR) | 0.59 | [0.43, 0.84] | 0.61 | [0.43, 0.77] | 0.71 |
| Baseline Medication, n (%) | | | | | |
| Antiplatelet Therapy | 191 | (94.1) | 29 | (67.4) | <0.001 |
| Aspirin | 183 | (90.1) | 21 | (48.8) | <0.001 |
| Clopidogrel | 87 | (42.9) | 13 | (30.2) | 0.174 |
| Statin | 171 | (84.2) | 35 | (81.4) | 0.817 |
| Beta Blocker | 124 | (61.1) | 26 | (60.5) | 1 |
| ACE/ARB | 109 | (53.7) | 25 | (58.1) | 0.717 |
| Baseline Lab Values | | | | | |
| Creatinine, mg/dl, median (IQR) | 1.0 | [0.8, 1.3] | 1.1 | [0.8, 1.6] | 0.444 |

ABI = ankle-brachial index; ACE/ARB = angiotensin-converting enzyme/angiotensin receptor blocker; BMI = body mass index; CAD = coronary artery disease; CVA = cerebral vascular accident; CVD = cardiovascular disease; MI = myocardial infarction; VTE = venous thromboembolism; COPD = Chronic obstructive pulmonary disease; IQR = inter-quartile range.

### Example 4. Platelet Hyperreactivity and Clinical Events

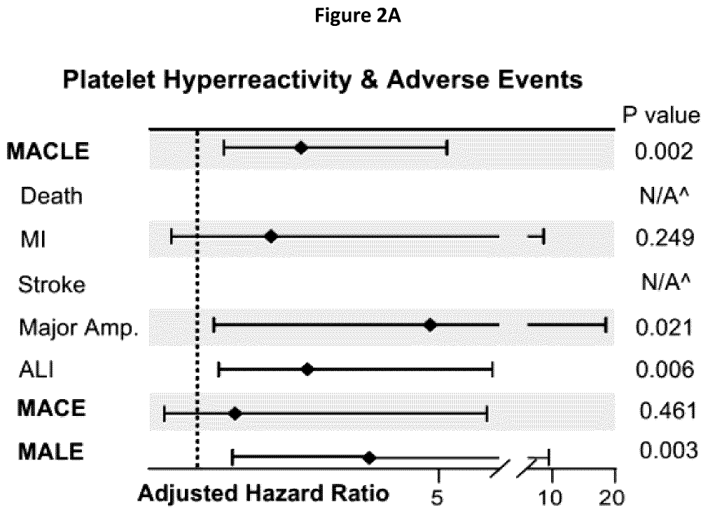

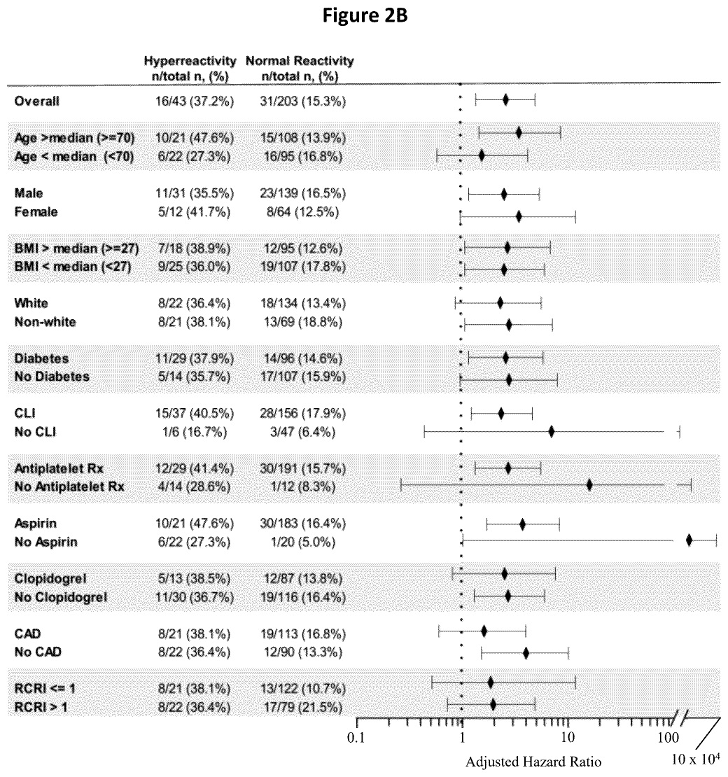

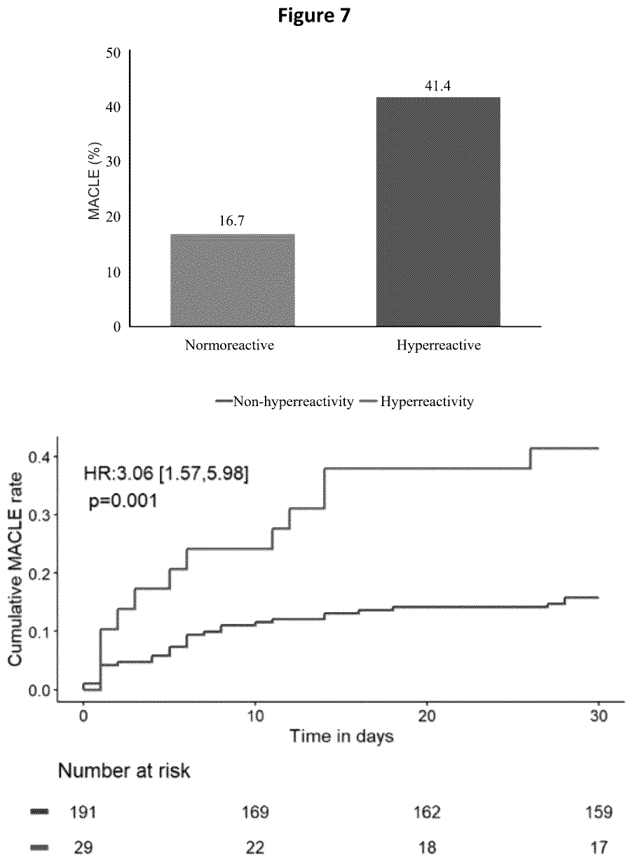

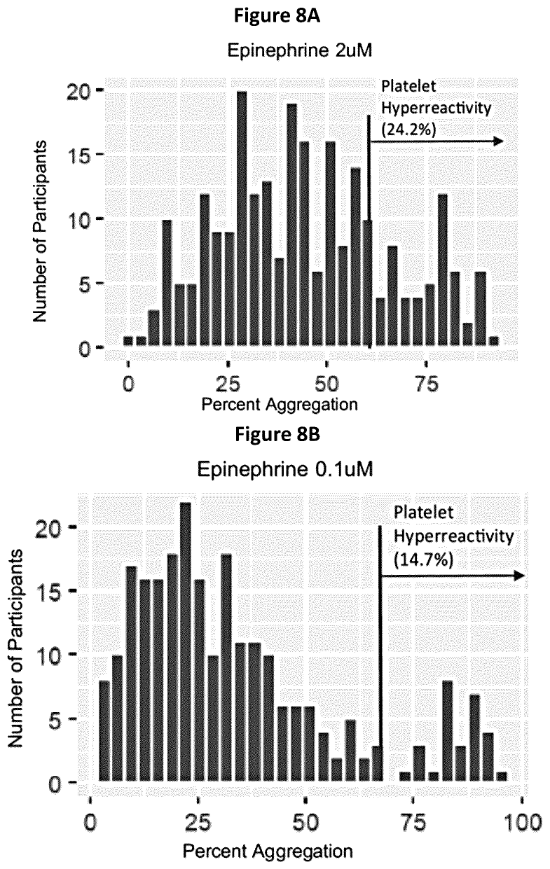

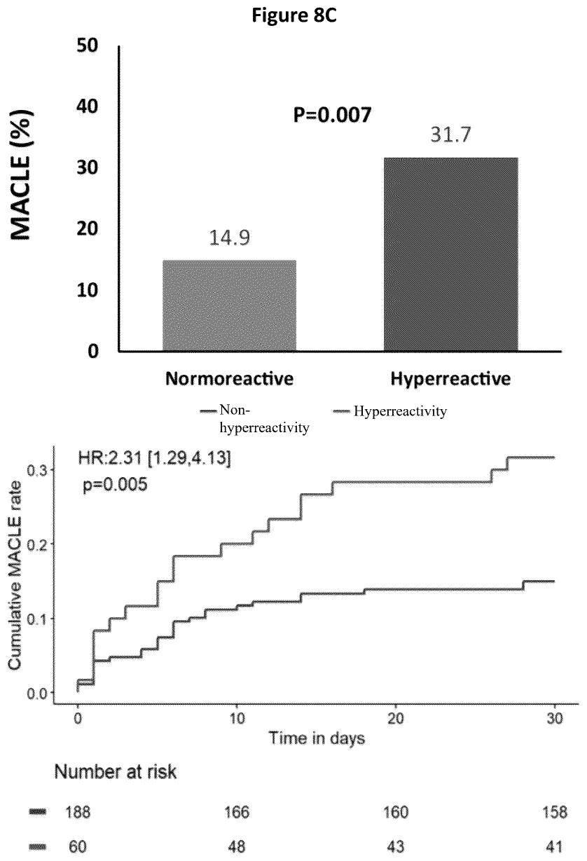

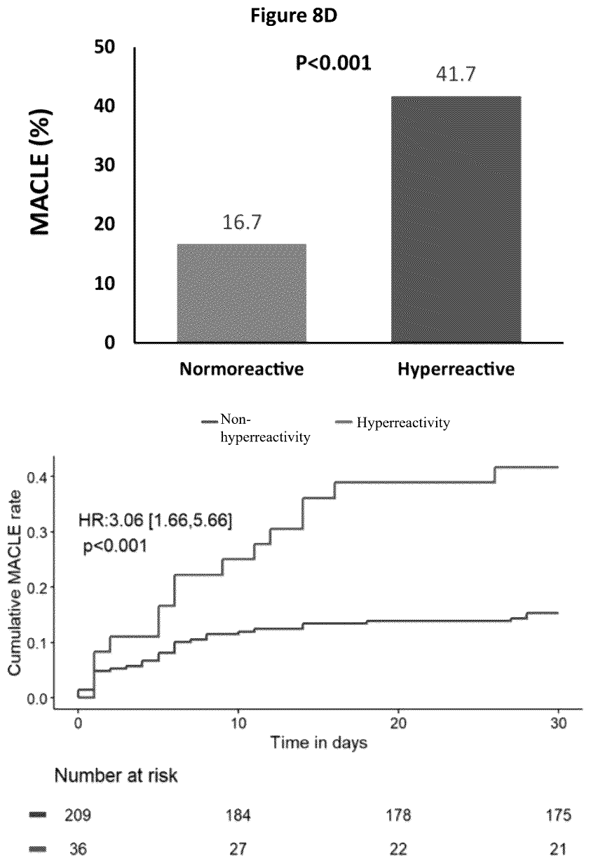

Individuals with a hyperreactive platelet phenotype in response to 0.4 μM epinephrine had a higher incidence of 30-day MACLE (37.2% vs. 15.3% in those with aggregation≤60%, p=0.002; FIGS. 1E, 1F). The frequency of events, including individual components of the composite MACLE endpoint, is shown in Table 6, stratified by those with and without a hyperreactive platelet phenotype in response to submaximal epinephrine. After multivariable adjustment for age, sex, race/ethnicity, body mass index, diabetes, carotid artery disease, prior stroke, and CLI, platelet hyperreactivity was associated with a nearly 3-fold increased risk of MACLE (adjusted HR [aHR] 2.76, 95% CI 1.5 to 5.1, p=0.002, FIG. 2A, Table 6). After excluding 31 (10.7%) patients who were not on antiplatelet therapy at the time of blood collection, a hyperreactive platelet phenotype remained independently associated with MACLE (41.4% vs. 16.7%; HR 3.06, 95% CI 1.57 to 5.98, p=0.001; FIG. 7). Platelet hyperreactivity in response to a range of epinephrine concentrations was consistently associated with MACLE after multivariable adjustment (FIG. 8; Table 7).

TABLE 6
Major adverse cardiac and limb events at 30 days according to platelet
hyperreactivity (>60% aggregation to 0.4 μM epinephrine).

| Adverse | Platelet | | No Platelet | | | | |
|---|---|---|---|---|---|---|---|
| Events, n | Hyperreactivity | | Hyperreactivity | | P | Adjusted* HR | |
| (%) | (n = 43) | | (n= 203) | | value | (95% CI) | |
| MACLE | 16 | (37.2) | 31 | (15.3) | 0.002 | 2.76 | [1.51, 5.05] |
| Death | 0 | (0.0) | 1 | (0.5) | 1 | NA\hat{ } | |
| MI | 4 | (9.3) | 6 | (3.0) | 0.077 | 3.19 | [0.90, 11.32] |
| Stroke | 0 | (0.0) | 1 | (0.5) | 1 | NA\hat{ } | |
| Major | 5 | (11.6) | 5 | (2.5) | 0.019 | 4.89 | [1.42, 16.89] |
| Amputa- | | | | | | | |
| tion | | | | | | | |
| Acute | 12 | (27.9) | 24 | (11.8) | 0.007 | 2.59 | [1.30, 5.19] |
| Limb | | | | | | | |
| Ischemia | | | | | | | |
| MACE | 4 | (9.3) | 8 | (3.9) | 0.232 | 2.38 | [0.72, 7.90] |
| MALE | 9 | (20.9) | 15 | (7.4) | 0.015 | 3.03 | [1.32, 6.92] |

MACLE, major adverse cardiac and limb events, defined as a composite of death, myocardial infarction (MI), stroke, major amputation, or acute limb ischemia leading to revascularization;
MACE, major adverse cardiac events, defined as a composite of death, MI, stroke;
MALE, major adverse limb events, defined as a composite of major amputation or acute limb ischemia leading to revascularization;
ALI, acute limb ischemia
*Adjusted for age, sex, race, ethnicity, BMI, diabetes, carotid artery disease, prior stroke, and CLI.
\hat{ }too few events to estimate

TABLE 7
Major adverse cardiovascular and limb events stratified by platelet
hyperreactivity (60% aggregation) in response to different doses of
epinephrine at 180 seconds, 300 seconds, and maximum aggregation.

| | | | P- | Unadjusted | P- | Adjusted* | P- |
|---|---|---|---|---|---|---|---|
| | | MACLE | value | HR | value | HR | value |
| Epinephrine 2 μM | | | | | | | |
| 180 s | ≤60 | 28 (14.9) | 0.007 | 2.31 | 0.0049 | 2.32 | 0.006 |
| | >60 | 19 (31.7) | | [1.29, 4.13] | | [1.27, 4.23] | |
| 300 s | ≤60 | 21 (13.4) | 0.006 | 2.33 | 0.0039 | 2.37 | 0.0041 |
| | >60 | 26 (28.6) | | [1.31, 4.15] | | [1.32, 4.27] | |
| Maximum | ≤60 | 15 (13.4) | 0.062 | 1.85 | 0.0496 | 1.77 | 0.079 |
| | >60 | 32 (23.5) | | [1, 3.41] | | [0.94, 3.34] | |
| Epinephrine 0.4 μM | | | | | | | |
| 180 s | ≤60 | 36 (16.2) | 0.001 | 3.4 | 0.0004 | 3.39 | 0.0009 |
| | >60 | 11 (45.8) | | [1.73, 6.68] | | [1.64, 6.97] | |
| 300 s | ≤60 | 31 (15.4) | 0.004 | 2.58 | 0.0021 | 2.53 | 0.004 |
| | >60 | 16 (35.6) | | [1.41, 4.73] | | [1.35, 4.77] | |
| Maximum | ≤60 | 24 (14.2) | 0.006 | 2.33 | 0.0038 | 2.31 | 0.0051 |
| | >60 | 23 (29.9) | | [1.32, 4.13] | | [1.29, 4.14] | |
| Epinephrine 0.1 μM | | | | | | | |
| 180 s | ≤60 | 44 (18.5) | 0.131 | 2.58 | 0.1128 | 2.92 | 0.1228 |
| | >60 | 3 (42.9) | | [0.8, 8.31] | | [0.75, 11.35] | |
| 300 s | ≤60 | 42 (18.4) | 0.429 | 1.63 | 0.3035 | 1.5 | 0.4171 |
| | >60 | 5 (29.4) | | [0.64, 4.11] | | [0.56, 4.04] | |
| Maximum | ≤60 | 32 (15.3) | 0.001 | 3.06 | 0.0004 | 3.13 | 0.0005 |
| | >60 | 15 (41.7) | | [1.66, 5.66] | | [1.64, 5.97] | |

Multivariable adjusted hazard ratios for 30-day MACLE associated with platelet hyperreactivity are shown in FIG. 2B, stratified by other baseline characteristics. The association between platelet hyperreactivity and MACLE was directionally consistent across age, sex, CV risk, and antiplatelet therapies.

A hyperreactive platelet phenotype discriminates patients at risk of 30-day MACLE beyond commonly used perioperative cardiac risk models (e.g., RCRI) (27). The addition of platelet hyperreactivity to the RCRI model improved discrimination to predict the 30-day composite of MACLE (C-statistic for RCRI-0.59, 95% CI 0.51 to 0.68; C-statistic for RCRI plus platelet hyperreactivity-0.64, 95% CI 0.55 to 0.72; p=0.009). The addition of platelet hyperreactivity significantly improved both reclassification (NRI; 0.42, 95% CI 0.13 to 0.71; p=0.005) and the discrimination as calculated by the IDI (p=0.011).

### Example 5. Development of Platelet Reactivity ExpresSion Score (PRESS)

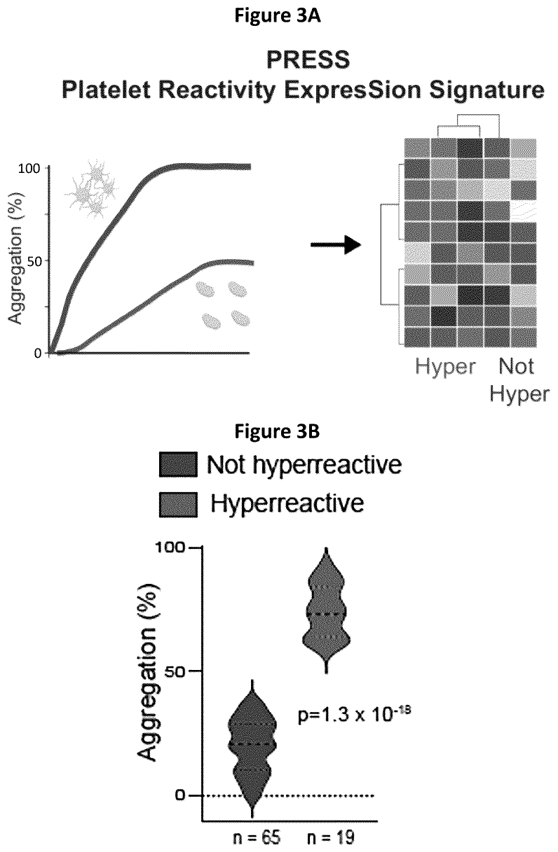

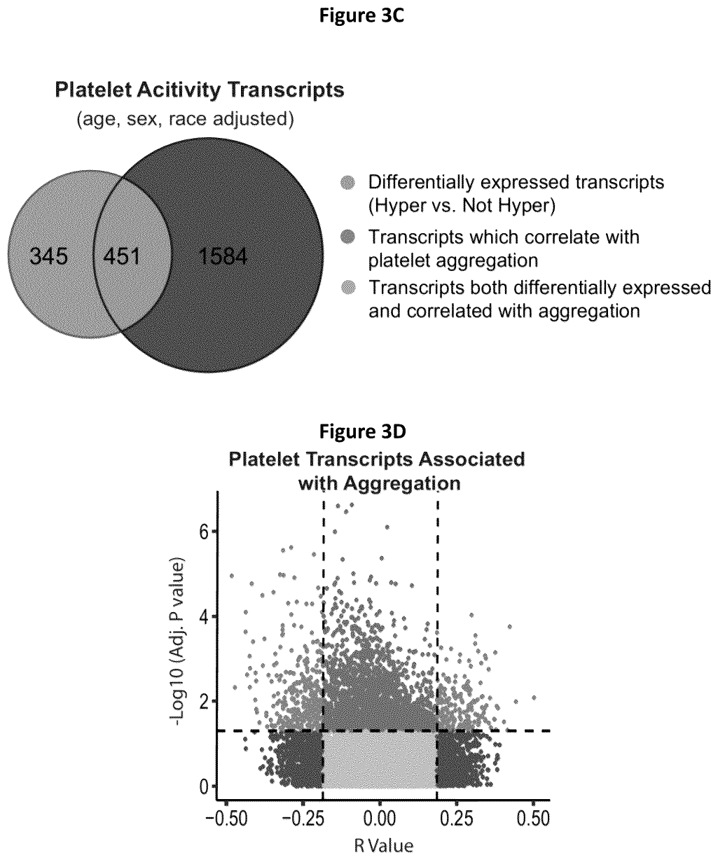

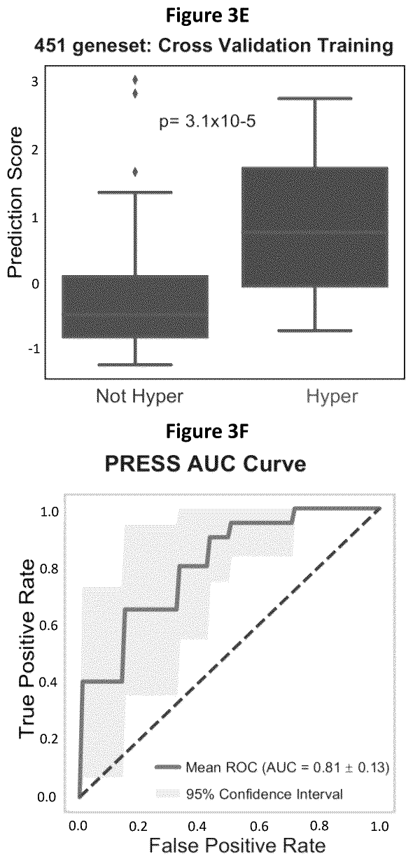

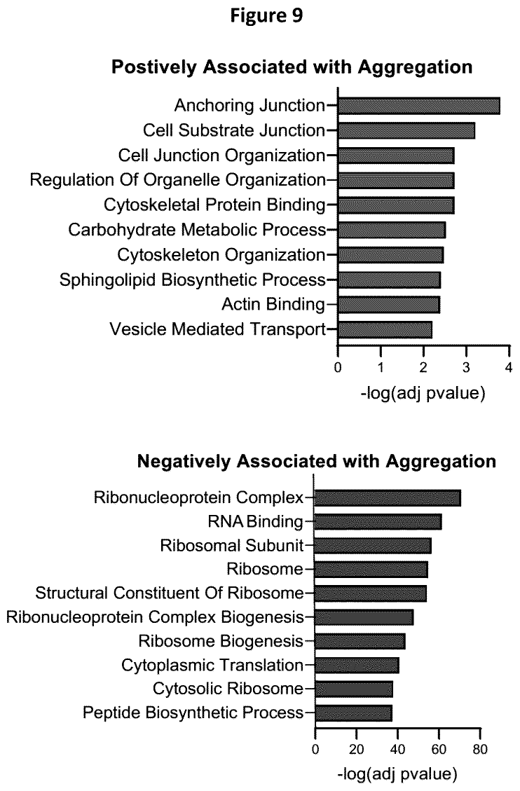

The platelet transcriptome is used to gain insight into platelet mechanisms (14,28,29), disease onset and progression (4,15,17,30), and is reproducible over time (31). Within the PACE cohort, 129 patients had platelet RNA sequencing from samples collected before LER (FIG. 6; FIG. 3A). Amongst these, 19 (22.6%) patients had a hyperreactive platelet response to epinephrine (0.4 μM, demographics FIG. 3B, Table 8). Following adjustment for age, sex, and race/ethnicity, differential expression analyses utilizing two statistical thresholds (DESeq2 and Wilcoxon signed-rank test) found that 796 transcripts were differentially expressed (p<0.05) in patients with a hyperreactive platelet phenotype. Gene set enrichment analysis (GSEA) revealed biological pathways linked to platelet activation enriched in subjects with hyperreactive platelets. In the basal state, pathways related to anchoring junctions, secretory granules, the cytoskeleton, ribosomes, and vesicle-mediated transport were enriched in platelets from hyperreactive subjects (FIG. 9). Among these 796 differentially expressed transcripts, 451 were significantly correlated (p<0.05) with percent aggregation to epinephrine (0.4 μM), with 174 transcripts positively and 277 transcripts negatively associated with platelet hyperreactivity (FIGS. 3C, 3D, Table 9). Using a combination of weighted expression values from these 451 genes, a Platelet Reactivity ExpresSion Score (PRESS) was generated for each participant. Following 10-fold cross-validation, PRESS discriminated patients from those without platelet hyperreactivity (AUC [cross-validation] 0.81, 95% CI 0.68 to 0.94). The weight used for these genes are shown in Table 10. A 76 gene subset has been determined and maintains excellent stratification of both platelet hyperreactivity and risk of CV events (genes and weights bolded in Tables 9-10, respectively).

TABLE 8
Baseline characteristics in PACE-PAD Subjects with platelet RNA-Seq

| | Normoreactive | | Hyperreactive | | P |
|---|---|---|---|---|---|
| | (n = 64) | | (n = 19) | | value |
| Age, mean (SD) | 68.64 | (10.85) | 69.79 | (10.70) | 0.685 |
| Female, n (% ) | 17 | (26.6) | 8 | (42.1) | 0.312 |
| Race | | | | | 0.032 |
| White | 47 | (73.4) | 8 | (42.1) | |
| Black | 12 | (18.8) | 6 | (31.6) | |
| Asian | 1 | (1.6) | 2 | (10.5) | |
| Other | 4 | (6.2) | 3 | (15.8) | |
| Ethnicity | | | | | 0.482 |
| Hispanic | 9 | (14.1) | 4 | (21.1) | |
| BMI, mean (SD) | 27.53 | (5.89) | 27.42 | (5.60) | 0.941 |
| Smoking | | | | | 0.622 |
| Current | 9 | (14.1) | 1 | (5.3) | |
| Former | 42 | (65.6) | 13 | (68.4) | |
| Never | 13 | (20.3) | 5 | (26.3) | |
| Family History of CVD | 21 | (33.3) | 6 | (33.3) | 1 |
| Clinical History | | | | | |
| CAD | 31 | (48.4) | 10 | (52.6) | 0.952 |
| Prior MI | 17 | (26.6) | 5 | (26.3) | 1 |
| Prior CVA | 12 | (18.8) | 5 | (26.3) | 0.694 |
| Carotid artery disease | 6 | (9.4) | 1 | (5.3) | 1 |
| VTE | 7 | (10.9) | 5 | (26.3) | 0.193 |
| Diabetes | 28 | (43.8) | 16 | (84.2) | 0.003 |
| COPD | 13 | (20.3) | 2 | (10.5) | 0.502 |
| Hypertension | 53 | (82.8) | 18 | (94.7) | 0.280 |
| Hyperlipidemia | 48 | (75.0) | 16 | (84.2) | 0.540 |
| Cancer | 9 | (14.1) | 6 | (31.6) | 0.161 |
| Heart Failure | | | | | |
| Indication | | | | | |
| Critical limb ischemia | 46 | (71.9) | 18 | (94.7) | 0.059 |
| Rest pain | 29 | (45.3) | 5 | (26.3) | 0.225 |
| Gangrene | 9 | (14.1) | 10 | (52.6) | 0.001 |
| Ulceration | 24 | (37.5) | 8 | (42.1) | 0.925 |
| Lowest ABI, median (IQR) | 0.54 | [0.39, 0.72] | 0.50 | [0.38, 0.62] | 0.532 |
| ABI in index limb, median (IQR) | 0.56 | [0.43, 0.79] | 0.54 | [0.38, 0.68] | 0.688 |
| Baseline Medication | | | | | |
| Antiplatelet Therapy | 64 | (100.0) | 19 | (100.0) | NA |
| Statin | 56 | (87.5) | 17 | (89.5) | 1 |
| Beta Blocker | 35 | (54.7) | 10 | (52.6) | 1 |
| ACE/ARB | 33 | (51.6) | 13 | (68.4) | 0.300 |
| Lab Values | | | | | |
| Baseline Creatinine | 1.00 | [0.90, 1.10] | 1.10 | [0.85, 1.40] | 0.418 |

ABI = ankle-brachial index; ACE/ARB = angiotensin-converting enzyme/angiotensin receptor blocker; BMI = body mass index; CAD = coronary artery disease; CVA = cerebral vascular accident; CVD = cardiovascular disease; MI = myocardial infarction; VTE = venous thromboembolism; COPD = Chronic obstructive pulmonary disease; IQR = inter-quartile range.

TABLE 9
PRESS Transcripts

| | | | | |
|---|---|---|---|---|
| AASDHPPT | DCTN6 | KHK | RAB13 | UBE2Z |
| ABHD17A | DCUN1D4 | KIAA0586 | RAB1A | UBE3B |
| ABHD17B | DDX3Y | KIAA1324L | RAB2B | UBE4B |
| ABHD18 | DDX6 | KIF3A | RAB3GAP2 | UFM1 |
| ACADSB | DENND4A | KIF3C | RAC1P4 | UHRF1BP1 |
| ACAP2 | DIS3L2 | KLF10 | RAD51AP1 | USP15 |
| ACER2 | DISP1 | KLHDC1 | RASA1 | USP39 |
| ACTR3C | DNAJB2 | KLHDC8B | RB1 | UTRN |
| AFF4 | DTWD1 | KLHL9 | RB1CC1 | UXS1 |
| AHR | DYRK4 | KLRA1P | RBM12B | VKORC1 |
| AK6 | EDEM3 | KRIT1 | RBM7 | VNN1 |
| ALCAM | EEF1AKMT2 | LACTB2 | RCHY1 | VPS13C |
| ALG10 | EIF2AK1 | LAMC1 | RGCC | VPS45 |
| ANGPT1 | ELAC1 | LCORL | RHOC | VRK1 |
| ANKRA2 | ELF1 | LINC00867 | RIC8A | WASF1 |
| ANXA11 | ELF2 | LINC00963 | RIOK2 | WDR1 |
| AP1B1 | EMC2 | LINC01218 | RNF138 | WDR13 |
| AP2B1 | EMCN | LINC02001 | RNF24 | WDR47 |
| AP2M1 | ENTHD1 | LINC02284 | RP9 | WWC3 |
| APOOL | ENY2 | LMBRD2 | RPL41 | XIAP |
| ARF1 | ERCC1 | LSM14B | RPTOR | XRN1 |
| ARFGEF1 | EVL | LYSMD3 | RRP15 | ZBTB1 |
| ARHGAP10 | F11R | LYST | RTN1 | ZBTB11 |
| ARHGAP5 | FAM13A | MAP1A | SAR1B | ZBTB4 |
| ARHGDIB | FAM189B | MAPKAPK2 | SCAMP1 | ZFR |
| ARHGEF12 | FAM229A | MBNL2 | SCFD1 | ZHX1-C8orf76 |
| ARID1A | FAM76B | MCEMP1 | SEC14L2 | ZNF134 |
| ARL1 | FAR1 | MECP2 | SEC22A | ZNF33A |
| ARPC1B | FBXL13 | MED16 | SEM1 | ZNF347 |
| ARV1 | FNBP1 | MEMO1P1 | SEPT7P7 | ZNF44 |
| ASS1P1 | FOXN2 | METTL7A | SFT2D1 | ZNF451 |
| ATF4 | FRG1 | MFAP3 | SFXN5 | ZNF460-AS1 |
| ATF4P4 | FTH1P10 | MGST2 | SGO2 | ZNF517 |
| ATF7IP | FTH1P11 | MICA | SGTB | ZNF706 |
| ATG2B | FTH1P12 | MINDY1 | SH3BP4 | ZNF804A |
| ATP6V0A2 | FTH1P2 | MLLT11 | SLC22A17 | AC004053.1 |
| ATXN7 | FTH1P20 | MORC3 | SLC25A24 | AC006213.2 |
| ATXN7L3 | FTH1P7 | MORF4L2 | SLC35B3 | AC006378.2 |
| BAG2 | FTL | MRPL15 | SLC3A2 | AC016642.1 |
| BAG4 | FTLP2 | MTREX | SLFN14 | AC062015.1 |
| BANK1 | FTLP3 | MYL6 | SLU7 | AC087481.3 |
| BAP1 | FUNDC2 | MYL6P5 | SMAD5 | AC092718.4 |
| BCR | FUNDC2P1 | MYLK | SMARCAD1 | AC093673.1 |
| BIRC3 | FUNDC2P2 | MYO18B | SMC2 | AC096577.1 |
| BLZF1 | FURIN | NAP1L4 | SMC6 | AC104248.1 |
| BMT2 | G6PD | NBEAP1 | SMIM10L1 | AC106795.2 |
| BSCL2 | GABPB1 | NCBP1 | SMIM15 | AC106865.1 |
| BSDC1 | GABRD | NCBP2 | SMIM3 | AC112200.2 |
| BTG3 | GADD45G | NCKIPSD | SMPD1 | AC118549.1 |
| BX537318.1 | GALM | NDUFB5 | SMU1 | AC120114.1 |
| C10orf88 | GINM1 | NFE2L2 | SNHG5 | AC131971.1 |
| C12orf29 | GIPC3 | NINJ1 | SNRPN | AC243829.1 |
| C17orf58 | GLRX3 | NMD3 | SPARC | AL049828.1 |
| C1orf198 | GNAI3 | NMI | SPC25 | AL133163.2 |
| C20orf96 | GPAT4 | NOMO2 | SPIN4 | AL136382.1 |
| C21orf91 | GPATCH2 | NPAT | SPOCD1 | AL137024.1 |
| C2orf68 | GPBP1 | NR1D2 | SPRY1 | AL139095.4 |
| C3orf38 | GPX7 | NSUN6 | SPSB4 | AL589743.1 |
| C5orf51 | GRK4 | NUTF2 | ST3GAL1 | AL606491.1 |
| C6orf106 | GSN | OGFRL1 | STAM2 | |
| CA7 | GSPT1 | ORAI2 | STIM1 | |
| CALCOCO1 | GTPBP8 | ORC5 | STIMATE | |
| CAPN1 | GUSB | OSBPL3 | STK40 | |
| CAPN2 | GUSBP11 | P2RX1 | SUGT1 | |
| CAVIN4 | H3F3B | P2RY10 | SUGT1P1 | |
| CCDC25 | H3F3C | PACSIN2 | SVIL | |
| CCDC82 | HAUS1 | PAFAH1B1 | TADA1 | |
| CCDC88C | HDAC2-AS2 | PAIP1 | TAF12 | |
| CCDC9B | HEMGN | PAPPA | TANK | |
| CCL18 | HIBCH | PAQR7 | TAX1BP1 | |
| CCNY | HINFP | PCSK6 | TAX1BP3 | |
| CD69 | HIST1H3C | PDS5B | TBC1D13 | |
| CDC37 | HLA-G | PEX3 | TBC1D20 | |
| CDKN2AIP | HLA-J | PGAM1 | TBC1D22A | |
| CDYL2 | HSBP1P2 | PGD | TCAIM | |
| CEP120 | HSPA2 | PHF3 | TCEANC | |
| CEP170 | IDE | PHF6 | TEX30 | |
| CHODL-AS1 | IER3IP1 | PI4K2B | TFAM | |
| CIZ1 | IFT57 | PI4KA | TGDS | |
| CKAP2L | IFT74 | PIAS1 | THNSL1 | |
| CLK1 | IGF2BP2 | PIGS | THUMPD2 | |
| CNOT7 | IL7 | PIK3R1 | TIPRL | |
| COBLL1 | IMPA1 | PIP5K1B | TMCO3 | |
| COCH | IMPA2 | PLA2G4C | TMEM106B | |
| COLEC12 | IMPACT | PLCH1 | TMEM11 | |
| COPS2 | ING4 | PLIN3 | TMEM164 | |
| COQ10B | INSIG2 | POMGNT2 | TNFAIP8 | |
| COQ5 | IPCEF1 | POT1 | TOMM40L | |
| CREB1 | IQCB1 | PPHLN1 | TOMM70 | |
| CRLF3 | IQUB | PPIL3 | TPI1P1 | |
| CROT | ITGB5 | PPIL4 | TPST2 | |
| CRYBA4 | JRKL | PPP1R2 | TRAPPC5 | |
| CTSA | JTB | PRKCA | TRBV4-1 | |
| CUL1 | KANSL1L | PRMT3 | TRMT1L | |
| CUX1 | KBTBD2 | PSMD10 | TRPC2 | |
| CXXC4 | KCTD2 | PTBP3 | TSPAN13 | |
| DAG1 | KDM3A | PTMAP10 | TTI2 | |
| DCAF16 | KHDRBS2 | PTMAP9 | U2SURP | |

TABLE 10
PRESS Transcript Weights

| Gene | AC006378.2 | AC087481.3 | AC106795.2 | AC112200.2 | AC131971.1 | AC243829.1 | ACTR3C | AL589743.1 | ALCAM | AP1B1 |
|---|---|---|---|---|---|---|---|---|---|---|
| rf0 | 0.0014 | 0 | 0.001851 | 0.003692 | 0.004384 | 0.004392 | 0.003101 | 0.003578 | 0.003064 | 0.002079 |
| rf1 | 0 | 0.010128 | 0 | 0 | 0 | 0.008553 | 0.002421 | 0.000477 | 0 | 0.005334 |
| rf2 | 0.004692 | 0.001983 | 0.000376 | 0 | 3.34E−17 | 0 | 0.005307 | 0.000361 | 0.003173 | 0.000749 |
| rf3 | 0.000344 | 0.002585 | 0.006443 | 0.000971 | 0.012316 | 0.001075 | 0.002914 | 0.000696 | 0.001147 | 0.001847 |
| rf4 | 0.001053 | 0.000703 | 0.000412 | 0.000958 | 0.002797 | 0 | 0.009159 | 0.000359 | 0.001987 | 0.003144 |
| rf5 | 0.001219 | 0.00149 | 0 | 0.002059 | 0.000384 | 0.002803 | 0.003976 | 0.002236 | 0 | 0.006656 |
| rf6 | 0.004211 | 0.001358 | 0 | 0.001548 | 0.000944 | 0.000721 | 0.008278 | 0.00473 | 0 | 0 |
| rf7 | 0 | 0.00345 | 0.000979 | 0.003893 | 0.007153 | 0.001589 | 0.002405 | 0.001033 | 0.002881 | 0.000718 |
| rf8 | 0 | 0.00752 | 0.00412 | 0.00565 | 0.016691 | 0.00207 | 0.007097 | 0.002293 | 0.002185 | 0.009046 |
| rf9 | 0.003124 | 0.000968 | 0.00221 | 0 | 0.006545 | 0.001437 | 0.00309 | 0.001814 | 0.002117 | 0.000352 |
| rf10 | 5.66E−18 | 0.002694 | 0.000486 | 0.003281 | 0.00622 | 0.003921 | 0.000767 | 0.004733 | 0.009295 | 0.007571 |
| rf11 | 0.00571 | 0.000696 | 0.006778 | 0 | 0.00033 | 0 | 0.002055 | 0.001785 | 0.005124 | 0 |
| rf12 | 0.010112 | 0.006891 | 0.001729 | 0.003377 | 0.000667 | 0.010914 | 0.000342 | 0.002308 | 0.004435 | 0.00376 |
| rf13 | 0.005009 | 0 | 0 | 0 | 0.000663 | 0 | 0.003695 | 0.003559 | 0.000377 | 0.000398 |
| rf14 | 0.0014 | 0 | 0.001851 | 0.003692 | 0.004384 | 0.004392 | 0.003101 | 0.003578 | 0.003064 | 0.002079 |
| rf15 | 0 | 0.010128 | 0 | 0 | 0 | 0.008553 | 0.002421 | 0.000477 | 0 | 0.005334 |
| rf16 | 0.004692 | 0.001983 | 0.000376 | 0 | 3.34E−17 | 0 | 0.005307 | 0.000361 | 0.003173 | 0.000749 |
| rf17 | 0.000344 | 0.002585 | 0.006443 | 0.000971 | 0.012316 | 0.001075 | 0.002914 | 0.000696 | 0.001147 | 0.001847 |
| rf18 | 0.001053 | 0.000703 | 0.000412 | 0.000958 | 0.002797 | 0 | 0.009159 | 0.000359 | 0.001987 | 0.003144 |
| rf19 | 0.001219 | 0.00149 | 0 | 0.002059 | 0.000384 | 0.002803 | 0.003976 | 0.002236 | 0 | 0.006656 |
| rf20 | 0.004211 | 0.001358 | 0 | 0.001548 | 0.000944 | 0.000721 | 0.008278 | 0.00473 | 0 | 0 |
| rf21 | 0 | 0.00345 | 0.000979 | 0.003893 | 0.007153 | 0.001589 | 0.002405 | 0.001033 | 0.002881 | 0.000718 |
| rf22 | 0 | 0.00752 | 0.00412 | 0.00565 | 0.016691 | 0.00207 | 0.007097 | 0.002293 | 0.002185 | 0.009046 |
| rf23 | 0.003124 | 0.000968 | 0.00221 | 0 | 0.006545 | 0.001437 | 0.00309 | 0.001814 | 0.002117 | 0.000352 |
| rf24 | 5.66E−18 | 0.002694 | 0.000486 | 0.003281 | 0.00622 | 0.003921 | 0.000767 | 0.004733 | 0.009295 | 0.007571 |
| rf25 | 0.00571 | 0.000696 | 0.006778 | 0 | 0.00033 | 0 | 0.002055 | 0.001785 | 0.005124 | 0 |
| rf26 | 0.010112 | 0.006891 | 0.001729 | 0.003377 | 0.000667 | 0.010914 | 0.000342 | 0.002308 | 0.004435 | 0.00376 |
| rf27 | 0.005009 | 0 | 0 | 0 | 0.000663 | 0 | 0.003695 | 0.003559 | 0.000377 | 0.000398 |
| extraTrees0 | 0.003484 | 0.003784 | 0.002689 | 0.00438 | 0.001456 | 0.001411 | 0.007629 | 0.003448 | 0.000559 | 0.001328 |
| extraTrees1 | 0.004812 | 0.002648 | 0.001571 | 0.001611 | 0.00235 | 0.002603 | 0.002684 | 0.001892 | 0.002326 | 0.004347 |
| extraTrees2 | 0.004662 | 0.003492 | 0.001642 | 0.000592 | 0.003208 | 0.00224 | 0.00794 | 0.003691 | 0.002241 | 0.007425 |
| extraTrees3 | 0.002176 | 0.001061 | 0.00274 | 0.003482 | 0.001884 | 0.002518 | 0.004363 | 0.001837 | 0.002656 | 0.003419 |
| extraTrees4 | 0.001479 | 0.001892 | 0.002989 | 0.000771 | 0.002802 | 0.001972 | 0.005694 | 0.003632 | 0.003159 | 0.000627 |
| extraTrees5 | 0.005667 | 0.00487 | 0.00237 | 0.001728 | 0.003818 | 0.004776 | 0.00275 | 0.004115 | 0.002923 | 0.000736 |
| extraTrees6 | 0.005437 | 0.003462 | 0.004619 | 0.003668 | 0.004594 | 0.003111 | 0.007899 | 0.001246 | 0.001382 | 0.005279 |
| extraTrees7 | 0.004416 | 0.007896 | 0.00282 | 0.002053 | 0.002652 | 0.004734 | 0.005115 | 0.001671 | 0.00175 | 0.002925 |
| extraTrees8 | 0.002059 | 0.000824 | 0.002975 | 0.000839 | 0.003608 | 0.004263 | 0.008872 | 0.001792 | 0.002485 | 0.002465 |
| extraTrees9 | 0.000577 | 0.003714 | 0.001076 | 0.002077 | 0.000983 | 0.007524 | 0.005286 | 0.002071 | 0.00388 | 0.002683 |
| extraTrees10 | 0.005272 | 0.006511 | 0.001096 | 0.002605 | 0.001615 | 0.002898 | 0.008697 | 0.001857 | 0.003044 | 0.002477 |
| extraTrees11 | 0.004748 | 0.006102 | 0.004083 | 0.001347 | 0.002783 | 0.00329 | 0.005649 | 0.003226 | 0.002061 | 0.001702 |
| extraTrees12 | 0.003883 | 0.00121 | 0.002379 | 0.000855 | 0.004936 | 0.007282 | 0.004369 | 0.003567 | 0.003789 | 0.004621 |
| extraTrees13 | 0.005012 | 0.001856 | 0.00266 | 0.001233 | 0.002174 | 0.007786 | 0.002488 | 0.003159 | 0.00162 | 0.006402 |
| extraTrees14 | 0.003484 | 0.003784 | 0.002689 | 0.00438 | 0.001456 | 0.001411 | 0.007629 | 0.003448 | 0.000559 | 0.001328 |
| extraTrees15 | 0.004812 | 0.002648 | 0.001571 | 0.001611 | 0.00235 | 0.002603 | 0.002684 | 0.001892 | 0.002326 | 0.004347 |
| extraTrees16 | 0.004662 | 0.003492 | 0.001642 | 0.000592 | 0.003208 | 0.00224 | 0.00794 | 0.003691 | 0.002241 | 0.007425 |
| extraTrees17 | 0.002176 | 0.001061 | 0.00274 | 0.003482 | 0.001884 | 0.002518 | 0.004363 | 0.001837 | 0.002656 | 0.003419 |
| extraTrees18 | 0.001479 | 0.001892 | 0.002989 | 0.000771 | 0.002802 | 0.001972 | 0.005694 | 0.003632 | 0.003159 | 0.000627 |
| extraTrees19 | 0.005667 | 0.00487 | 0.00237 | 0.001728 | 0.003818 | 0.004776 | 0.00275 | 0.004115 | 0.002923 | 0.000736 |
| extraTrees20 | 0.005437 | 0.003462 | 0.004619 | 0.003668 | 0.004594 | 0.003111 | 0.007899 | 0.001246 | 0.001382 | 0.005279 |
| extraTrees21 | 0.004416 | 0.007896 | 0.00282 | 0.002053 | 0.002652 | 0.004734 | 0.005115 | 0.001671 | 0.00175 | 0.002925 |
| extraTrees22 | 0.002059 | 0.000824 | 0.002975 | 0.000839 | 0.003608 | 0.004263 | 0.008872 | 0.001792 | 0.002485 | 0.002465 |
| extraTrees23 | 0.000577 | 0.003714 | 0.001076 | 0.002077 | 0.000983 | 0.007524 | 0.005286 | 0.002071 | 0.00388 | 0.002683 |
| extraTrees24 | 0.005272 | 0.006511 | 0.001096 | 0.002605 | 0.001615 | 0.002898 | 0.008697 | 0.001857 | 0.003044 | 0.002477 |
| extraTrees25 | 0.004748 | 0.006102 | 0.004083 | 0.001347 | 0.002783 | 0.00329 | 0.005649 | 0.003226 | 0.002061 | 0.001702 |
| extraTrees26 | 0.003883 | 0.00121 | 0.002379 | 0.000855 | 0.004936 | 0.007282 | 0.004369 | 0.003567 | 0.003789 | 0.004621 |
| extraTrees27 | 0.005012 | 0.001856 | 0.00266 | 0.001233 | 0.002174 | 0.007786 | 0.002488 | 0.003159 | 0.00162 | 0.006402 |
| gbc0 | 0 | 0 | 0 | 0.00024 | 0 | 0 | 9.58E−05 | 0.005256 | 0 | 0.002452 |
| gbc1 | 0.001557 | 0.010951 | 0.005932 | 0.003444 | 0.00189 | 4.99E−06 | 7.72E−05 | 0.000239 | 0.014502 | 0 |
| gbc2 | 2.38E−08 | 0.004251 | 1.81E−06 | 0.001438 | 0 | 0.01436 | 0 | 1.84E−05 | 0 | 5.69E−05 |
| gbc3 | 3.68E−05 | 0.008497 | 0 | 0 | 0 | 0.001908 | 0.000196 | 0.010879 | 0.000143 | 0.000313 |
| gbc4 | 0.001169 | 0 | 0.000365 | 0 | 0.024612 | 0 | 0.006334 | 0.00416 | 0 | 0 |
| gbc5 | 0.002092 | 0.015297 | 0 | 0.000245 | 0.014025 | 0 | 0.000386 | 0 | 0.000778 | 0 |
| gbc6 | 0.000122 | 0 | 0.000695 | 0 | 0.003059 | 0.004542 | 0.015904 | 6.19E−05 | 0 | 0 |
| gbc7 | 0.022097 | 0.016479 | 0.000149 | 0 | 0 | 0.000615 | 0.004669 | 0.006563 | 2.54E−06 | 0.001544 |
| gbc8 | 0.007333 | 0.000435 | 0.002146 | 0.000142 | 0.008517 | 0.004622 | 8.16E−05 | 5.31E−05 | 0 | 0 |
| gbc9 | 0 | 2.99E−05 | 1.99E−05 | 0 | 0.003371 | 0.001475 | 0.000644 | 1.87E−05 | 0 | 0 |
| gbc10 | 0.001662 | 0.004487 | 0.003258 | 1.96E−05 | 0 | 0.000443 | 0 | 0.000437 | 0.012412 | 0 |
| gbc11 | 0.001684 | 0.00223 | 0.000884 | 9.52E−06 | 0.000317 | 0.001598 | 0.000519 | 1.78E−05 | 0 | 8.85E−05 |
| gbc12 | 0.000369 | 0.030474 | 0.000315 | 2.47E−05 | 0.006015 | 0 | 0.00078 | 0.004407 | 0 | 0.002907 |
| gbc13 | 0 | 0.001244 | 0.01189 | 0.000341 | 0.017776 | 1.53E−05 | 0.00062 | 0.0011 | 0 | 9.50E−05 |
| gbc14 | 0 | 0 | 0 | 0.00024 | 0 | 0 | 9.58E−05 | 0.005256 | 0 | 0.002452 |
| gbc15 | 0.001557 | 0.010951 | 0.005932 | 0.003444 | 0.00189 | 4.99E−06 | 7.72E−05 | 0.000239 | 0.014502 | 0 |
| gbc16 | 2.38E−08 | 0.004251 | 1.81E−06 | 0.001438 | 0 | 0.01436 | 0 | 1.84E−05 | 0 | 5.69E−05 |
| gbc17 | 3.68E−05 | 0.008497 | 0 | 0 | 0 | 0.001908 | 0.000196 | 0.010879 | 0.000143 | 0.000313 |
| gbc18 | 0.001169 | 0 | 0.000365 | 0 | 0.024612 | 0 | 0.006334 | 0.00416 | 0 | 0 |
| gbc19 | 0.002092 | 0.015297 | 0 | 0.000245 | 0.014025 | 0 | 0.000386 | 0 | 0.000778 | 0 |
| gbc20 | 0.000122 | 0 | 0.000695 | 0 | 0.003059 | 0.004542 | 0.015904 | 6.19E−05 | 0 | 0 |
| gbc21 | 0.022097 | 0.016479 | 0.000149 | 0 | 0 | 0.000615 | 0.004669 | 0.006563 | 2.54E−06 | 0.001544 |
| gbc22 | 0.007333 | 0.000435 | 0.002146 | 0.000142 | 0.008517 | 0.004622 | 8.16E−05 | 5.31E−05 | 0 | 0 |
| gbc23 | 0 | 2.99E−05 | 1.99E−05 | 0 | 0.003371 | 0.001475 | 0.000644 | 1.87E−05 | 0 | 0 |
| gbc24 | 0.001662 | 0.004487 | 0.003258 | 1.96E−05 | 0 | 0.000443 | 0 | 0.000437 | 0.012412 | 0 |
| gbc25 | 0.001684 | 0.00223 | 0.000884 | 9.52E−06 | 0.000317 | 0.001598 | 0.000519 | 1.78E−05 | 0 | 8.85E−05 |
| gbc26 | 0.000369 | 0.030474 | 0.000315 | 2.47E−05 | 0.006015 | 0 | 0.00078 | 0.004407 | 0 | 0.002907 |
| gbc27 | 0 | 0.001244 | 0.01189 | 0.000341 | 0.017776 | 1.53E−05 | 0.00062 | 0.0011 | 0 | 9.50E−05 |
| ada0 | 0 | 0 | 0.02 | 0 | 0.02 | 0 | 0.04 | 0 | 0 | 0 |
| ada1 | 0 | 0 | 0.02 | 0 | 0.02 | 0 | 0.04 | 0 | 0 | 0 |
| ada2 | 0 | 0 | 0.02 | 0 | 0.02 | 0 | 0.04 | 0 | 0 | 0 |
| ada3 | 0 | 0 | 0.02 | 0 | 0.02 | 0 | 0.04 | 0 | 0 | 0 |
| ada4 | 0 | 0 | 0.02 | 0 | 0.02 | 0 | 0.04 | 0 | 0 | 0 |
| ada5 | 0 | 0 | 0.02 | 0 | 0.02 | 0 | 0.04 | 0 | 0 | 0 |
| ada6 | 0 | 0 | 0.02 | 0 | 0.02 | 0 | 0.04 | 0 | 0 | 0 |
| ada7 | 0 | 0 | 0.02 | 0 | 0.02 | 0 | 0.04 | 0 | 0 | 0 |
| ada8 | 0 | 0 | 0.02 | 0 | 0.02 | 0 | 0.04 | 0 | 0 | 0 |
| ada9 | 0 | 0 | 0.02 | 0 | 0.02 | 0 | 0.04 | 0 | 0 | 0 |
| ada10 | 0 | 0 | 0.02 | 0 | 0.02 | 0 | 0.04 | 0 | 0 | 0 |
| ada11 | 0 | 0 | 0.02 | 0 | 0.02 | 0 | 0.04 | 0 | 0 | 0 |
| ada12 | 0 | 0 | 0.02 | 0 | 0.02 | 0 | 0.04 | 0 | 0 | 0 |
| ada13 | 0 | 0 | 0.02 | 0 | 0.02 | 0 | 0.04 | 0 | 0 | 0 |
| ada14 | 0 | 0 | 0.02 | 0 | 0.02 | 0 | 0.04 | 0 | 0 | 0 |
| ada15 | 0 | 0 | 0.02 | 0 | 0.02 | 0 | 0.04 | 0 | 0 | 0 |
| ada16 | 0 | 0 | 0.02 | 0 | 0.02 | 0 | 0.04 | 0 | 0 | 0 |
| ada17 | 0 | 0 | 0.02 | 0 | 0.02 | 0 | 0.04 | 0 | 0 | 0 |
| ada18 | 0 | 0 | 0.02 | 0 | 0.02 | 0 | 0.04 | 0 | 0 | 0 |
| ada19 | 0 | 0 | 0.02 | 0 | 0.02 | 0 | 0.04 | 0 | 0 | 0 |
| ada20 | 0 | 0 | 0.02 | 0 | 0.02 | 0 | 0.04 | 0 | 0 | 0 |
| ada21 | 0 | 0 | 0.02 | 0 | 0.02 | 0 | 0.04 | 0 | 0 | 0 |
| ada22 | 0 | 0 | 0.02 | 0 | 0.02 | 0 | 0.04 | 0 | 0 | 0 |
| ada23 | 0 | 0 | 0.02 | 0 | 0.02 | 0 | 0.04 | 0 | 0 | 0 |
| ada24 | 0 | 0 | 0.02 | 0 | 0.02 | 0 | 0.04 | 0 | 0 | 0 |
| ada25 | 0 | 0 | 0.02 | 0 | 0.02 | 0 | 0.04 | 0 | 0 | 0 |
| ada26 | 0 | 0 | 0.02 | 0 | 0.02 | 0 | 0.04 | 0 | 0 | 0 |
| ada27 | 0 | 0 | 0.02 | 0 | 0.02 | 0 | 0.04 | 0 | 0 | 0. |

| Gene | ARHGAP10 | ARV1 | BCR | BMT2 | BX537318.1 | C20orf96 | CA7 | CCNY | CDYL2 | CIZ1 |
|---|---|---|---|---|---|---|---|---|---|---|
| rf0 | 0.001394 | 0.001241 | 6.80E−18 | 0.005101 | 0.000372 | 0.00071 | 0.000562 | 0.005746 | 0.00096 | 0.006481 |
| rf1 | 0.001246 | 0 | 0 | 0.00598 | 1.52E−18 | 0 | 0.000357 | 0.006717 | 0.002008 | 0.002672 |
| rf2 | 0.000354 | 0.001426 | 0 | 0 | 0.002731 | 0 | 0.000414 | 0.007695 | 0.006264 | 0.00666 |
| rf3 | 0 | 0.000339 | 0.001518 | 0.001785 | 0.000525 | 0.001935 | 0 | 0.003483 | 0.00094 | 0.006728 |
| rf4 | 0.000683 | 0.001063 | 0.003337 | 0.001698 | 0.005209 | 0 | 0.001719 | 0.002336 | 5.66E−18 | 0.002435 |
| rf5 | 0.011603 | 0 | 8.54E−05 | 0.001667 | 0.005308 | 0.001838 | 0.000384 | 0.004491 | 0 | 0 |
| rf6 | 0.001844 | 0 | 0.001435 | 0 | 0 | 0.00111 | 0.002306 | 0.005221 | 0.000917 | 0.001424 |
| rf7 | 0.001096 | 0 | 0 | 0 | 0.007729 | 0.002222 | 0.000966 | 0.001041 | 0.000638 | 0.002831 |
| rf8 | 0 | 0 | 0.003531 | 0.004092 | 0.002296 | 0.009464 | 0.000288 | 0.003242 | 0.000576 | 0.001812 |
| rf9 | 0.003854 | 1.89E−17 | 0.002302 | 0.011291 | 0.003208 | 0.00083 | 0.001957 | 0.001848 | 0.002071 | 0.001042 |
| rf10 | 0.000359 | 0.001707 | 0.000311 | 0.001745 | 0.002279 | 0.000799 | 0 | 0.001043 | 0 | 0.003198 |
| rf11 | 0.002807 | 0.001384 | 0.003602 | 0 | 0.007023 | 0.004218 | 0.00426 | 0 | 0 | 0.002471 |
| rf12 | 0.001504 | 0.004097 | 0.001885 | 0.00098 | 0.006754 | 0.001149 | 0 | 0.001732 | 0.001278 | 0.004643 |
| rf13 | 0.00414 | 0.001431 | 0.001908 | 0.002737 | 0 | 0.001981 | 4.16E−18 | 0 | 0 | 0 |
| rf14 | 0.001394 | 0.001241 | 6.80E−18 | 0.005101 | 0.000372 | 0.00071 | 0.000562 | 0.005746 | 0.00096 | 0.006481 |
| rf15 | 0.001246 | 0 | 0 | 0.00598 | 1.52E−18 | 0 | 0.000357 | 0.006717 | 0.002008 | 0.002672 |
| rf16 | 0.000354 | 0.001426 | 0 | 0 | 0.002731 | 0 | 0.000414 | 0.007695 | 0.006264 | 0.00666 |
| rf17 | 0 | 0.000339 | 0.001518 | 0.001785 | 0.000525 | 0.001935 | 0 | 0.003483 | 0.00094 | 0.006728 |
| rf18 | 0.000683 | 0.001063 | 0.003337 | 0.001698 | 0.005209 | 0 | 0.001719 | 0.002336 | 5.66E−18 | 0.002435 |
| rf19 | 0.011603 | 0 | 8.54E−05 | 0.001667 | 0.005308 | 0.001838 | 0.000384 | 0.004491 | 0 | 0 |
| rf20 | 0.001844 | 0 | 0.001435 | 0 | 0 | 0.00111 | 0.002306 | 0.005221 | 0.000917 | 0.001424 |
| rf21 | 0.001096 | 0 | 0 | 0 | 0.007729 | 0.002222 | 0.000966 | 0.001041 | 0.000638 | 0.002831 |
| rf22 | 0 | 0 | 0.003531 | 0.004092 | 0.002296 | 0.009464 | 0.000288 | 0.003242 | 0.000576 | 0.001812 |
| rf23 | 0.003854 | 1.89E−17 | 0.002302 | 0.011291 | 0.003208 | 0.00083 | 0.001957 | 0.001848 | 0.002071 | 0.001042 |
| rf24 | 0.000359 | 0.001707 | 0.000311 | 0.001745 | 0.002279 | 0.000799 | 0 | 0.001043 | 0 | 0.003198 |
| rf25 | 0.002807 | 0.001384 | 0.003602 | 0 | 0.007023 | 0.004218 | 0.00426 | 0 | 0 | 0.002471 |
| rf26 | 0.001504 | 0.004097 | 0.001885 | 0.00098 | 0.006754 | 0.001149 | 0 | 0.001732 | 0.001278 | 0.004643 |
| rf27 | 0.00414 | 0.001431 | 0.001908 | 0.002737 | 0 | 0.001981 | 4.16E−18 | 0 | 0 | 0 |
| extraTrees0 | 0.001836 | 0.002742 | 0.001971 | 0.000859 | 0.003827 | 0.002043 | 0.001542 | 0.001432 | 0.002981 | 0.006318 |
| extraTrees1 | 0.000526 | 0.001246 | 0.000638 | 0.000476 | 0.00418 | 0.001111 | 0.006608 | 0.002778 | 0.00163 | 0.004445 |
| extraTrees2 | 0.001593 | 0.001995 | 0.00159 | 0.003503 | 0.002293 | 0.003322 | 0.002846 | 0.002271 | 0.002847 | 0.001095 |
| extraTrees3 | 0.001395 | 0.001981 | 0.002281 | 0 | 0.002301 | 0.001145 | 0.003579 | 0.001256 | 0.002393 | 0.001707 |
| extraTrees4 | 0.004198 | 0.001367 | 0.002575 | 0 | 0.001702 | 0.002771 | 0.001678 | 0.001529 | 0.000775 | 0.001396 |
| extraTrees5 | 0.001681 | 0.001651 | 0.003596 | 0.000136 | 0.006803 | 0.000492 | 0.002404 | 0.001025 | 0.000615 | 0.003591 |
| extraTrees6 | 0.004105 | 0.000507 | 0.001049 | 0.001989 | 0.00441 | 0.002883 | 0.003719 | 0.001815 | 0.003973 | 0.003261 |
| extraTrees7 | 0.00357 | 0.000273 | 0.002974 | 0.003473 | 0.001163 | 0.002265 | 0.00391 | 0.003144 | 0.002523 | 0.008639 |
| extraTrees8 | 0.003153 | 0.000245 | 0.000293 | 0.002089 | 0.001759 | 0.00464 | 0.001803 | 1.71E−18 | 0.003511 | 0.006456 |
| extraTrees9 | 0.002387 | 0.000268 | 0.000612 | 0.003143 | 0.004838 | 0.002327 | 0.000667 | 0.005886 | 0.004626 | 0.004819 |
| extraTrees10 | 0.004404 | 0.000299 | 0.001826 | 0.001259 | 0.002041 | 0.001381 | 0.003816 | 0.002584 | 0.001212 | 0.002565 |
| extraTrees11 | 0.00473 | 0.004359 | 0.001359 | 0.001924 | 0.003277 | 0.000726 | 0.00232 | 0 | 0.002566 | 0.004493 |
| extraTrees12 | 0.004059 | 0.002825 | 0.001545 | 0 | 0.002923 | 0.001788 | 0.001857 | 0.005417 | 0.003631 | 0.002011 |
| extraTrees13 | 0.001994 | 0.001373 | 0.001115 | 0.004269 | 0.000887 | 0.002514 | 0.002437 | 0.002422 | 0.001579 | 0.008089 |
| extraTrees14 | 0.001836 | 0.002742 | 0.001971 | 0.000859 | 0.003827 | 0.002043 | 0.001542 | 0.001432 | 0.002981 | 0.006318 |
| extraTrees15 | 0.000526 | 0.001246 | 0.000638 | 0.000476 | 0.00418 | 0.001111 | 0.006608 | 0.002778 | 0.00163 | 0.004445 |
| extraTrees16 | 0.001593 | 0.001995 | 0.00159 | 0.003503 | 0.002293 | 0.003322 | 0.002846 | 0.002271 | 0.002847 | 0.001095 |
| extraTrees17 | 0.001395 | 0.001981 | 0.002281 | 0 | 0.002301 | 0.001145 | 0.003579 | 0.001256 | 0.002393 | 0.001707 |
| extraTrees18 | 0.004198 | 0.001367 | 0.002575 | 0 | 0.001702 | 0.002771 | 0.001678 | 0.001529 | 0.000775 | 0.001396 |
| extraTrees19 | 0.001681 | 0.001651 | 0.003596 | 0.000136 | 0.006803 | 0.000492 | 0.002404 | 0.001025 | 0.000615 | 0.003591 |
| extraTrees20 | 0.004105 | 0.000507 | 0.001049 | 0.001989 | 0.00441 | 0.002883 | 0.003719 | 0.001815 | 0.003973 | 0.003261 |
| extraTrees21 | 0.00357 | 0.000273 | 0.002974 | 0.003473 | 0.001163 | 0.002265 | 0.00391 | 0.003144 | 0.002523 | 0.008639 |
| extraTrees22 | 0.003153 | 0.000245 | 0.000293 | 0.002089 | 0.001759 | 0.00464 | 0.001803 | 1.71E−18 | 0.003511 | 0.006456 |
| extraTrees23 | 0.002387 | 0.000268 | 0.000612 | 0.003143 | 0.004838 | 0.002327 | 0.000667 | 0.005886 | 0.004626 | 0.004819 |
| extraTrees24 | 0.004404 | 0.000299 | 0.001826 | 0.001259 | 0.002041 | 0.001381 | 0.003816 | 0.002584 | 0.001212 | 0.002565 |
| extraTrees25 | 0.00473 | 0.004359 | 0.001359 | 0.001924 | 0.003277 | 0.000726 | 0.00232 | 0 | 0.002566 | 0.004493 |
| extraTrees26 | 0.004059 | 0.002825 | 0.001545 | 0 | 0.002923 | 0.001788 | 0.001857 | 0.005417 | 0.003631 | 0.002011 |
| extraTrees27 | 0.001994 | 0.001373 | 0.001115 | 0.004269 | 0.000887 | 0.002514 | 0.002437 | 0.002422 | 0.001579 | 0.008089 |
| gbc0 | 0 | 0 | 2.41E−05 | 0 | 0 | 0 | 0.006639 | 0.004997 | 0.000142 | 0.012733 |
| gbc1 | 0 | 0 | 0.000149 | 7.31E−05 | 0.013941 | 2.72E−05 | 0 | 0.000141 | 0.00024 | 0.000503 |
| gbc2 | 0 | 0 | 0 | 0 | 6.12E−05 | 0 | 0.004017 | 0.007097 | 0.001277 | 0.001035 |
| gbc3 | 0.000276 | 0 | 0.002131 | 0.001097 | 0.000184 | 0 | 0 | 0 | 0.000805 | 8.98E−05 |
| gbc4 | 3.12E−05 | 0 | 7.71E−05 | 0.020742 | 8.38E−05 | 0.010175 | 0 | 0.033006 | 0.00444 | 0 |
| gbc5 | 0 | 2.17E−05 | 0.001793 | 0.00807 | 0.006214 | 0.017607 | 0 | 0.001425 | 0.003179 | 0 |
| gbc6 | 3.01E−05 | 0 | 0.001313 | 0 | 0 | 0 | 5.07E−05 | 4.40E−05 | 5.37E−05 | 0.007079 |
| gbc7 | 0 | 0 | 0.010228 | 3.55E−05 | 0.000255 | 0.001427 | 0.000732 | 0.002235 | 0.002151 | 0 |
| gbc8 | 0.007952 | 0.002735 | 0 | 0.003541 | 0.002123 | 0.000704 | 0.001697 | 0 | 1.02E−05 | 0 |
| gbc9 | 0.001742 | 0.000531 | 0 | 0 | 0.001247 | 5.07E−05 | 2.63E−06 | 0 | 0.012337 | 0 |
| gbc10 | 0 | 0.004635 | 0.000104 | 0 | 6.70E−05 | 0 | 0 | 0.003399 | 0 | 0 |
| gbc11 | 0.000162 | 0 | 7.92E−06 | 0 | 0.002044 | 0.006919 | 0.016193 | 0.009192 | 0.001336 | 3.36E−05 |
| gbc12 | 0 | 0 | 0 | 0 | 0 | 0.015491 | 1.76E−05 | 0 | 0.008644 | 0 |
| gbc13 | 0 | 0 | 4.17E−05 | 2.14E−05 | 2.91E−06 | 0.000626 | 0.012086 | 0.002641 | 0.015934 | 0.00072 |
| gbc14 | 0 | 0 | 2.41E−05 | 0 | 0 | 0 | 0.006639 | 0.004997 | 0.000142 | 0.012733 |
| gbc15 | 0 | 0 | 0.000149 | 7.31E−05 | 0.013941 | 2.72E−05 | 0 | 0.000141 | 0.00024 | 0.000503 |
| gbc16 | 0 | 0 | 0 | 0 | 6.12E−05 | 0 | 0.004017 | 0.007097 | 0.001277 | 0.001035 |
| gbc17 | 0.000276 | 0 | 0.002131 | 0.001097 | 0.000184 | 0 | 0 | 0 | 0.000805 | 8.98E−05 |
| gbc18 | 3.12E−05 | 0 | 7.71E−05 | 0.020742 | 8.38E−05 | 0.010175 | 0 | 0.033006 | 0.00444 | 0 |
| gbc19 | 0 | 2.17E−05 | 0.001793 | 0.00807 | 0.006214 | 0.017607 | 0 | 0.001425 | 0.003179 | 0 |
| gbc20 | 3.01E−05 | 0 | 0.001313 | 0 | 0 | 0 | 5.07E−05 | 4.40E−05 | 5.37E−05 | 0.007079 |
| gbc21 | 0 | 0 | 0.010228 | 3.55E−05 | 0.000255 | 0.001427 | 0.000732 | 0.002235 | 0.002151 | 0 |
| gbc22 | 0.007952 | 0.002735 | 0 | 0.003541 | 0.002123 | 0.000704 | 0.001697 | 0 | 1.02E−05 | 0 |
| gbc23 | 0.001742 | 0.000531 | 0 | 0 | 0.001247 | 5.07E−05 | 2.63E−06 | 0 | 0.012337 | 0 |
| gbc24 | 0 | 0.004635 | 0.000104 | 0 | 6.70E−05 | 0 | 0 | 0.003399 | 0 | 0 |
| gbc25 | 0.000162 | 0 | 7.92E−06 | 0 | 0.002044 | 0.006919 | 0.016193 | 0.009192 | 0.001336 | 3.36E−05 |
| gbc26 | 0 | 0 | 0 | 0 | 0 | 0.015491 | 1.76E−05 | 0 | 0.008644 | 0 |
| gbc27 | 0 | 0 | 4.17E−05 | 2.14E−05 | 2.91E−06 | 0.000626 | 0.012086 | 0.002641 | 0.015934 | 0.00072 |
| ada0 | 0 | 0 | 0 | 0 | 0.08 | 0 | 0 | 0 | 0 | 0 |
| ada1 | 0 | 0 | 0 | 0 | 0.08 | 0 | 0 | 0 | 0 | 0 |
| ada2 | 0 | 0 | 0 | 0 | 0.08 | 0 | 0 | 0 | 0 | 0 |
| ada3 | 0 | 0 | 0 | 0 | 0.08 | 0 | 0 | 0 | 0 | 0 |
| ada4 | 0 | 0 | 0 | 0 | 0.08 | 0 | 0 | 0 | 0 | 0 |
| ada5 | 0 | 0 | 0 | 0 | 0.08 | 0 | 0 | 0 | 0 | 0 |
| ada6 | 0 | 0 | 0 | 0 | 0.08 | 0 | 0 | 0 | 0 | 0 |
| ada7 | 0 | 0 | 0 | 0 | 0.08 | 0 | 0 | 0 | 0 | 0 |
| ada8 | 0 | 0 | 0 | 0 | 0.08 | 0 | 0 | 0 | 0 | 0 |
| ada9 | 0 | 0 | 0 | 0 | 0.08 | 0 | 0 | 0 | 0 | 0 |
| ada10 | 0 | 0 | 0 | 0 | 0.08 | 0 | 0 | 0 | 0 | 0 |
| ada11 | 0 | 0 | 0 | 0 | 0.08 | 0 | 0 | 0 | 0 | 0 |
| ada12 | 0 | 0 | 0 | 0 | 0.08 | 0 | 0 | 0 | 0 | 0 |
| ada13 | 0 | 0 | 0 | 0 | 0.08 | 0 | 0 | 0 | 0 | 0 |
| ada14 | 0 | 0 | 0 | 0 | 0.08 | 0 | 0 | 0 | 0 | 0 |
| ada15 | 0 | 0 | 0 | 0 | 0.08 | 0 | 0 | 0 | 0 | 0 |
| ada16 | 0 | 0 | 0 | 0 | 0.08 | 0 | 0 | 0 | 0 | 0 |
| ada17 | 0 | 0 | 0 | 0 | 0.08 | 0 | 0 | 0 | 0 | 0 |
| ada18 | 0 | 0 | 0 | 0 | 0.08 | 0 | 0 | 0 | 0 | 0 |
| ada19 | 0 | 0 | 0 | 0 | 0.08 | 0 | 0 | 0 | 0 | 0 |
| ada20 | 0 | 0 | 0 | 0 | 0.08 | 0 | 0 | 0 | 0 | 0 |
| ada21 | 0 | 0 | 0 | 0 | 0.08 | 0 | 0 | 0 | 0 | 0 |
| ada22 | 0 | 0 | 0 | 0 | 0.08 | 0 | 0 | 0 | 0 | 0 |
| ada23 | 0 | 0 | 0 | 0 | 0.08 | 0 | 0 | 0 | 0 | 0 |
| ada24 | 0 | 0 | 0 | 0 | 0.08 | 0 | 0 | 0 | 0 | 0 |
| ada25 | 0 | 0 | 0 | 0 | 0.08 | 0 | 0 | 0 | 0 | 0 |
| ada26 | 0 | 0 | 0 | 0 | 0.08 | 0 | 0 | 0 | 0 | 0 |
| ada27 | 0 | 0 | 0 | 0 | 0.08 | 0 | 0 | 0 | 0 | 0. |

| Gene | COQ5 | CRYBA4 | CTSA | DDX3Y | DISP1 | DYRK4 | EVL | FAM189B | FBXL13 | GPX7 |
|---|---|---|---|---|---|---|---|---|---|---|
| rf0 | 0.006932 | 0.001509 | 0 | 0 | 0.005929 | 4.88E−18 | 0.000748 | 0.001524 | 0.003845 | 0.009296 |
| rf1 | 0.000352 | 0.003595 | 0.00375 | 0.000341 | 0.0043 | 0.003081 | 0.001554 | 6.46E−19 | 0.002262 | 1.45E−18 |
| rf2 | 0.00545 | 0.002921 | 0.000465 | 0.004228 | 0 | 0 | 0.002205 | 0.00168 | 0.001548 | 0.0011 |
| rf3 | 0.000372 | 0.001049 | 0 | 0.006179 | 0.001835 | 0.007908 | 0.001198 | 1.60E−18 | 0.003557 | 0.001112 |
| rf4 | 0.00249 | 0.00365 | 0.001639 | 0.000367 | 0.006369 | 0 | 1.65E−18 | 0.00141 | 0.002067 | 0.001006 |
| rf5 | 0.01289 | 0.0029 | 0 | 0.01151 | 0.0026 | 0.004359 | 0.001006 | 0.00173 | 0.002066 | 0 |
| rf6 | 0.008559 | 0 | 0 | 0.009356 | 0 | 0.003694 | 0 | 0 | 0.000665 | 0.006238 |
| rf7 | 0.000531 | 0.004846 | 0 | 0 | 0.000685 | 0.003659 | 0.000965 | 0.00205 | 0 | 3.07E−17 |
| rf8 | 0.000748 | 0.001676 | 0.002906 | 0 | 0.002671 | 0.004605 | 0.001067 | 0 | 0.002975 | 0.001573 |
| rf9 | 0.002782 | 0.001908 | 1.17E−18 | 0.003872 | 0.002431 | 0.000748 | 0.001127 | 0.007968 | 0.001477 | 0.001008 |
| rf10 | 0.001691 | 0.001705 | 0.000359 | 0.000549 | 0.002715 | 0.003065 | 0.000903 | 0.002014 | 0.001683 | 0.003429 |
| rf11 | 0.00129 | 0.000674 | 0.000293 | 0 | 0.002555 | 0.010048 | 0 | 0.002439 | 0.008588 | 0.001716 |
| rf12 | 0.001864 | 0.008042 | 0 | 0.000759 | 0.002266 | 0.002467 | 0.002654 | 0 | 0.003362 | 0 |
| rf13 | 0.004777 | 0.003648 | 0 | 0.006031 | 5.41E−18 | 0.002105 | 0 | 0.002796 | 0.00034 | 0.006048 |
| rf14 | 0.006932 | 0.001509 | 0 | 0 | 0.005929 | 4.88E−18 | 0.000748 | 0.001524 | 0.003845 | 0.009296 |
| rf15 | 0.000352 | 0.003595 | 0.00375 | 0.000341 | 0.0043 | 0.003081 | 0.001554 | 6.46E−19 | 0.002262 | 1.45E−18 |
| rf16 | 0.00545 | 0.002921 | 0.000465 | 0.004228 | 0 | 0 | 0.002205 | 0.00168 | 0.001548 | 0.0011 |
| rf17 | 0.000372 | 0.001049 | 0 | 0.006179 | 0.001835 | 0.007908 | 0.001198 | 1.60E−18 | 0.003557 | 0.001112 |
| rf18 | 0.00249 | 0.00365 | 0.001639 | 0.000367 | 0.006369 | 0 | 1.65E−18 | 0.00141 | 0.002067 | 0.001006 |
| rf19 | 0.01289 | 0.0029 | 0 | 0.01151 | 0.0026 | 0.004359 | 0.001006 | 0.00173 | 0.002066 | 0 |
| rf20 | 0.008559 | 0 | 0 | 0.009356 | 0 | 0.003694 | 0 | 0 | 0.000665 | 0.006238 |
| rf21 | 0.000531 | 0.004846 | 0 | 0 | 0.000685 | 0.003659 | 0.000965 | 0.00205 | 0 | 3.07E−17 |
| rf22 | 0.000748 | 0.001676 | 0.002906 | 0 | 0.002671 | 0.004605 | 0.001067 | 0 | 0.002975 | 0.001573 |
| rf23 | 0.002782 | 0.001908 | 1.17E−18 | 0.003872 | 0.002431 | 0.000748 | 0.001127 | 0.007968 | 0.001477 | 0.001008 |
| rf24 | 0.001691 | 0.001705 | 0.000359 | 0.000549 | 0.002715 | 0.003065 | 0.000903 | 0.002014 | 0.001683 | 0.003429 |
| rf25 | 0.00129 | 0.000674 | 0.000293 | 0 | 0.002555 | 0.010048 | 0 | 0.002439 | 0.008588 | 0.001716 |
| rf26 | 0.001864 | 0.008042 | 0 | 0.000759 | 0.002266 | 0.002467 | 0.002654 | 0 | 0.003362 | 0 |
| rf27 | 0.004777 | 0.003648 | 0 | 0.006031 | 5.41E−18 | 0.002105 | 0 | 0.002796 | 0.00034 | 0.006048 |
| extraTrees0 | 0.007681 | 0.005943 | 0.001417 | 0.006664 | 0.002806 | 0.005577 | 0.002757 | 3.05E−05 | 0.000779 | 0.002223 |
| extraTrees1 | 0.005689 | 0.004311 | 0.000214 | 0.004775 | 0.002607 | 0.00126 | 0.001568 | 0.000273 | 0.001601 | 0.003409 |
| extraTrees2 | 0.00577 | 0.003259 | 0.001631 | 0.002689 | 0.00384 | 0.002751 | 0.002527 | 0.002239 | 0.003656 | 0.002022 |
| extraTrees3 | 0.005765 | 0.008823 | 0.000253 | 0.00602 | 0.001867 | 0.001726 | 0.002407 | 0.001148 | 0.000816 | 0.001613 |
| extraTrees4 | 0.004575 | 0.002872 | 0.004635 | 0.003856 | 0.003193 | 0.004279 | 0.005702 | 0.001646 | 0.001525 | 0.004337 |
| extraTrees5 | 0.007403 | 0.003043 | 0.000319 | 0.001477 | 0.003549 | 0.001996 | 0.002852 | 0.000222 | 0.001687 | 0.001073 |
| extraTrees6 | 0.003944 | 0.003819 | 0.001632 | 0.00107 | 0.003453 | 0.00287 | 0.003943 | 0.00173 | 0.000953 | 0.003182 |
| extraTrees7 | 0.004614 | 0.005523 | 0.000293 | 0.004801 | 0.00369 | 0.003682 | 0.003046 | 0.002426 | 0.000561 | 0.002613 |
| extraTrees8 | 0.005587 | 0.002999 | 0.001913 | 0.000932 | 0.002316 | 0.005796 | 0.002679 | 0.002638 | 0.002667 | 0.004172 |
| extraTrees9 | 0.00293 | 0.002767 | 0.000648 | 0 | 0.004457 | 0.003042 | 0.004198 | 0.00028 | 0.002655 | 0.002886 |
| extraTrees10 | 0.005423 | 0.00466 | 0.001401 | 0.002177 | 0.001055 | 0.007751 | 0.007464 | 0.001233 | 0.001464 | 0.005856 |
| extraTrees11 | 0.007303 | 0.004136 | 0 | 0.005314 | 0.005527 | 0.004118 | 0.002992 | 0.000462 | 0.002877 | 0.002651 |
| extraTrees12 | 0.006671 | 0.002485 | 0.001649 | 0.002903 | 0.002466 | 0.004828 | 0.004317 | 0.00188 | 0.000984 | 0.003279 |
| extraTrees13 | 0.007807 | 0.001886 | 0.00025 | 0.002511 | 0.005573 | 0.006797 | 0.004263 | 0.001451 | 0.002639 | 0.002596 |
| extraTrees14 | 0.007681 | 0.005943 | 0.001417 | 0.006664 | 0.002806 | 0.005577 | 0.002757 | 3.05E−05 | 0.000779 | 0.002223 |
| extraTrees15 | 0.005689 | 0.004311 | 0.000214 | 0.004775 | 0.002607 | 0.00126 | 0.001568 | 0.000273 | 0.001601 | 0.003409 |
| extraTrees16 | 0.00577 | 0.003259 | 0.001631 | 0.002689 | 0.00384 | 0.002751 | 0.002527 | 0.002239 | 0.003656 | 0.002022 |
| extraTrees17 | 0.005765 | 0.008823 | 0.000253 | 0.00602 | 0.001867 | 0.001726 | 0.002407 | 0.001148 | 0.000816 | 0.001613 |
| extraTrees18 | 0.004575 | 0.002872 | 0.004635 | 0.003856 | 0.003193 | 0.004279 | 0.005702 | 0.001646 | 0.001525 | 0.004337 |
| extraTrees19 | 0.007403 | 0.003043 | 0.000319 | 0.001477 | 0.003549 | 0.001996 | 0.002852 | 0.000222 | 0.001687 | 0.001073 |
| extraTrees20 | 0.003944 | 0.003819 | 0.001632 | 0.00107 | 0.003453 | 0.00287 | 0.003943 | 0.00173 | 0.000953 | 0.003182 |
| extraTrees21 | 0.004614 | 0.005523 | 0.000293 | 0.004801 | 0.00369 | 0.003682 | 0.003046 | 0.002426 | 0.000561 | 0.002613 |
| extraTrees22 | 0.005587 | 0.002999 | 0.001913 | 0.000932 | 0.002316 | 0.005796 | 0.002679 | 0.002638 | 0.002667 | 0.004172 |
| extraTrees23 | 0.00293 | 0.002767 | 0.000648 | 0 | 0.004457 | 0.003042 | 0.004198 | 0.00028 | 0.002655 | 0.002886 |
| extraTrees24 | 0.005423 | 0.00466 | 0.001401 | 0.002177 | 0.001055 | 0.007751 | 0.007464 | 0.001233 | 0.001464 | 0.005856 |
| extraTrees25 | 0.007303 | 0.004136 | 0 | 0.005314 | 0.005527 | 0.004118 | 0.002992 | 0.000462 | 0.002877 | 0.002651 |
| extraTrees26 | 0.006671 | 0.002485 | 0.001649 | 0.002903 | 0.002466 | 0.004828 | 0.004317 | 0.00188 | 0.000984 | 0.003279 |
| extraTrees27 | 0.007807 | 0.001886 | 0.00025 | 0.002511 | 0.005573 | 0.006797 | 0.004263 | 0.001451 | 0.002639 | 0.002596 |
| gbc0 | 0.031166 | 0.000117 | 2.71E−05 | 0.016529 | 0.011501 | 0.001527 | 0.003116 | 2.24E−05 | 4.32E−05 | 0 |
| gbc1 | 0.001037 | 0.008009 | 0 | 0.000151 | 0.0032 | 0.003161 | 0 | 0.000683 | 0 | 0.041702 |
| gbc2 | 4.25E−05 | 0 | 0 | 0.002216 | 0.01339 | 0.000746 | 0.014888 | 0 | 0 | 0 |
| gbc3 | 0.002402 | 0.001981 | 0.014133 | 2.47E−05 | 0.001218 | 0.003557 | 2.00E−06 | 0 | 0.000547 | 0.000589 |
| gbc4 | 0.000187 | 3.90E−05 | 0.000237 | 5.21E−05 | 0.005075 | 0.001016 | 0 | 0.015198 | 0 | 0.006661 |
| gbc5 | 0.002181 | 0 | 0 | 0.000251 | 0 | 0 | 5.31E−05 | 0 | 0 | 0.007442 |
| gbc6 | 0.017607 | 0.000174 | 0 | 0.001739 | 1.21E−06 | 0.012189 | 0.001376 | 0.002291 | 0.000862 | 0.002252 |
| gbc7 | 0.004698 | 0 | 0.000124 | 0 | 0.000132 | 2.98E−06 | 0.016188 | 0.003355 | 0.004091 | 7.67E−05 |
| gbc8 | 0.028329 | 0 | 1.25E−05 | 0 | 0.017547 | 0.005375 | 0 | 0 | 3.89E−06 | 0.005641 |
| gbc9 | 0.000738 | 0 | 0 | 0.017416 | 0.016851 | 0.000175 | 0.003659 | 0.011915 | 0 | 0 |
| gbc10 | 1.71E−05 | 0.000869 | 0 | 0 | 0.011856 | 0.002686 | 0.001442 | 0.002349 | 0 | 0 |
| gbc11 | 0 | 0.016617 | 0 | 0 | 0.015968 | 0 | 0.00552 | 2.64E−05 | 0.003635 | 0 |
| gbc12 | 0.031392 | 1.67E−06 | 0 | 0.000885 | 0 | 0.012989 | 0 | 0.001157 | 0 | 0.000172 |
| gbc13 | 0.018595 | 5.30E−05 | 0 | 0.026337 | 0.002676 | 1.69E−05 | 0 | 0.002328 | 0 | 0 |
| gbc14 | 0.031166 | 0.000117 | 2.71E−05 | 0.016529 | 0.011501 | 0.001527 | 0.003116 | 2.24E−05 | 4.32E−05 | 0 |
| gbc15 | 0.001037 | 0.008009 | 0 | 0.000151 | 0.0032 | 0.003161 | 0 | 0.000683 | 0 | 0.041702 |
| gbc16 | 4.25E−05 | 0 | 0 | 0.002216 | 0.01339 | 0.000746 | 0.014888 | 0 | 0 | 0 |
| gbc17 | 0.002402 | 0.001981 | 0.014133 | 2.47E−05 | 0.001218 | 0.003557 | 2.00E−06 | 0 | 0.000547 | 0.000589 |
| gbc18 | 0.000187 | 3.90E−05 | 0.000237 | 5.21E−05 | 0.005075 | 0.001016 | 0 | 0.015198 | 0 | 0.006661 |
| gbc19 | 0.002181 | 0 | 0 | 0.000251 | 0 | 0 | 5.31E−05 | 0 | 0 | 0.007442 |
| gbc20 | 0.017607 | 0.000174 | 0 | 0.001739 | 1.21E−06 | 0.012189 | 0.001376 | 0.002291 | 0.000862 | 0.002252 |
| gbc21 | 0.004698 | 0 | 0.000124 | 0 | 0.000132 | 2.98E−06 | 0.016188 | 0.003355 | 0.004091 | 7.67E−05 |
| gbc22 | 0.028329 | 0 | 1.25E−05 | 0 | 0.017547 | 0.005375 | 0 | 0 | 3.89E−06 | 0.005641 |
| gbc23 | 0.000738 | 0 | 0 | 0.017416 | 0.016851 | 0.000175 | 0.003659 | 0.011915 | 0 | 0 |
| gbc24 | 1.71E−05 | 0.000869 | 0 | 0 | 0.011856 | 0.002686 | 0.001442 | 0.002349 | 0 | 0 |
| gbc25 | 0 | 0.016617 | 0 | 0 | 0.015968 | 0 | 0.00552 | 2.64E−05 | 0.003635 | 0 |
| gbc26 | 0.031392 | 1.67E−06 | 0 | 0.000885 | 0 | 0.012989 | 0 | 0.001157 | 0 | 0.000172 |
| gbc27 | 0.018595 | 5.30E−05 | 0 | 0.026337 | 0.002676 | 1.69E−05 | 0 | 0.002328 | 0 | 0 |
| ada0 | 0.02 | 0 | 0 | 0 | 0 | 0.02 | 0 | 0 | 0 | 0 |
| ada1 | 0.02 | 0 | 0 | 0 | 0 | 0.02 | 0 | 0 | 0 | 0 |
| ada2 | 0.02 | 0 | 0 | 0 | 0 | 0.02 | 0 | 0 | 0 | 0 |
| ada3 | 0.02 | 0 | 0 | 0 | 0 | 0.02 | 0 | 0 | 0 | 0 |
| ada4 | 0.02 | 0 | 0 | 0 | 0 | 0.02 | 0 | 0 | 0 | 0 |
| ada5 | 0.02 | 0 | 0 | 0 | 0 | 0.02 | 0 | 0 | 0 | 0 |
| ada6 | 0.02 | 0 | 0 | 0 | 0 | 0.02 | 0 | 0 | 0 | 0 |
| ada7 | 0.02 | 0 | 0 | 0 | 0 | 0.02 | 0 | 0 | 0 | 0 |
| ada8 | 0.02 | 0 | 0 | 0 | 0 | 0.02 | 0 | 0 | 0 | 0 |
| ada9 | 0.02 | 0 | 0 | 0 | 0 | 0.02 | 0 | 0 | 0 | 0 |
| ada10 | 0.02 | 0 | 0 | 0 | 0 | 0.02 | 0 | 0 | 0 | 0 |
| ada11 | 0.02 | 0 | 0 | 0 | 0 | 0.02 | 0 | 0 | 0 | 0 |
| ada12 | 0.02 | 0 | 0 | 0 | 0 | 0.02 | 0 | 0 | 0 | 0 |
| ada13 | 0.02 | 0 | 0 | 0 | 0 | 0.02 | 0 | 0 | 0 | 0 |
| ada14 | 0.02 | 0 | 0 | 0 | 0 | 0.02 | 0 | 0 | 0 | 0 |
| ada15 | 0.02 | 0 | 0 | 0 | 0 | 0.02 | 0 | 0 | 0 | 0 |
| ada16 | 0.02 | 0 | 0 | 0 | 0 | 0.02 | 0 | 0 | 0 | 0 |
| ada17 | 0.02 | 0 | 0 | 0 | 0 | 0.02 | 0 | 0 | 0 | 0 |
| ada18 | 0.02 | 0 | 0 | 0 | 0 | 0.02 | 0 | 0 | 0 | 0 |
| ada19 | 0.02 | 0 | 0 | 0 | 0 | 0.02 | 0 | 0 | 0 | 0 |
| ada20 | 0.02 | 0 | 0 | 0 | 0 | 0.02 | 0 | 0 | 0 | 0 |
| ada21 | 0.02 | 0 | 0 | 0 | 0 | 0.02 | 0 | 0 | 0 | 0 |
| ada22 | 0.02 | 0 | 0 | 0 | 0 | 0.02 | 0 | 0 | 0 | 0 |
| ada23 | 0.02 | 0 | 0 | 0 | 0 | 0.02 | 0 | 0 | 0 | 0 |
| ada24 | 0.02 | 0 | 0 | 0 | 0 | 0.02 | 0 | 0 | 0 | 0 |
| ada25 | 0.02 | 0 | 0 | 0 | 0 | 0.02 | 0 | 0 | 0 | 0 |
| ada26 | 0.02 | 0 | 0 | 0 | 0 | 0.02 | 0 | 0 | 0 | 0 |
| ada27 | 0.02 | 0 | 0 | 0 | 0 | 0.02 | 0 | 0 | 0 | 0. |

| Gene | HSBP1P2 | HSPA2 | IMPA2 | IPCEF1 | IQUB | KANSL1L | KCTD2 | KIF3A | LAMC1 | MCEMP1 |
|---|---|---|---|---|---|---|---|---|---|---|
| rf0 | 0.003973 | 0.011134 | 0 | 0.000609 | 0.004882 | 0.00993 | 0.005989 | 0.006905 | 0.01407 | 0 |
| rf1 | 0.003448 | 0.002149 | 0.003502 | 0 | 0.004258 | 0.0046 | 0 | 0 | 0.006178 | 0.000919 |
| rf2 | 0.001474 | 0.006595 | 0.011255 | 0.000672 | 0.003835 | 0.01548 | 0.00349 | 0.001772 | 0 | 0.001804 |
| rf3 | 0.004656 | 0.003296 | 0 | 0.005097 | 0.004919 | 0.0023 | 0.005263 | 0.002953 | 0.006653 | 0.001739 |
| rf4 | 0.010548 | 0.009675 | 0.003987 | 0.003355 | 0.00604 | 0.002492 | 0.000415 | 0.010728 | 0.002169 | 0.004693 |
| rf5 | 0.002932 | 0.003596 | 0.0043 | 0.001418 | 0.012995 | 0.000687 | 0.004289 | 0.016098 | 0.008912 | 0.001073 |
| rf6 | 0 | 0.000917 | 0 | 0 | 0.008205 | 0.004526 | 0.005699 | 0.007015 | 0.008067 | 0 |
| rf7 | 0.006148 | 0.00265 | 0.001536 | 0.004695 | 3.39E−18 | 0.005718 | 0.002272 | 0.001204 | 0 | 0.001898 |
| rf8 | 0.003882 | 0.010533 | 0.0091 | 0.003548 | 0.00282 | 0.00461 | 0 | 0.015828 | 0.0088 | 0.000371 |
| rf9 | 0.003997 | 2.51E−05 | 0 | 0 | 0 | 0.006206 | 0.00271 | 0.009381 | 0.001151 | 0 |
| rf10 | 0.002957 | 0.00187 | 3.27E−18 | 0.00821 | 0.000328 | 0.004059 | 0.001839 | 0.001744 | 0.003827 | 0.001454 |
| rf11 | 0.000988 | 0.009754 | 0.009499 | 0 | 0.001297 | 0.003889 | 0.003388 | 0.005177 | 0 | 0.001151 |
| rf12 | 0.002077 | 0.00648 | 0.001992 | 0.001829 | 0.005133 | 0.002422 | 0.002755 | 0.002848 | 0.009692 | 0.000926 |
| rf13 | 0 | 0 | 0.004836 | 0.002918 | 0.003922 | 0 | 0.000499 | 0.004864 | 0.001758 | 0.003741 |
| rf14 | 0.003973 | 0.011134 | 0 | 0.000609 | 0.004882 | 0.00993 | 0.005989 | 0.006905 | 0.01407 | 0 |
| rf15 | 0.003448 | 0.002149 | 0.003502 | 0 | 0.004258 | 0.0046 | 0 | 0 | 0.006178 | 0.000919 |
| rf16 | 0.001474 | 0.006595 | 0.011255 | 0.000672 | 0.003835 | 0.01548 | 0.00349 | 0.001772 | 0 | 0.001804 |
| rf17 | 0.004656 | 0.003296 | 0 | 0.005097 | 0.004919 | 0.0023 | 0.005263 | 0.002953 | 0.006653 | 0.001739 |
| rf18 | 0.010548 | 0.009675 | 0.003987 | 0.003355 | 0.00604 | 0.002492 | 0.000415 | 0.010728 | 0.002169 | 0.004693 |
| rf19 | 0.002932 | 0.003596 | 0.0043 | 0.001418 | 0.012995 | 0.000687 | 0.004289 | 0.016098 | 0.008912 | 0.001073 |
| rf20 | 0 | 0.000917 | 0 | 0 | 0.008205 | 0.004526 | 0.005699 | 0.007015 | 0.008067 | 0 |
| rf21 | 0.006148 | 0.00265 | 0.001536 | 0.004695 | 3.39E−18 | 0.005718 | 0.002272 | 0.001204 | 0 | 0.001898 |
| rf22 | 0.003882 | 0.010533 | 0.0091 | 0.003548 | 0.00282 | 0.00461 | 0 | 0.015828 | 0.0088 | 0.000371 |
| rf23 | 0.003997 | 2.51E−05 | 0 | 0 | 0 | 0.006206 | 0.00271 | 0.009381 | 0.001151 | 0 |
| rf24 | 0.002957 | 0.00187 | 3.27E−18 | 0.00821 | 0.000328 | 0.004059 | 0.001839 | 0.001744 | 0.003827 | 0.001454 |
| rf25 | 0.000988 | 0.009754 | 0.009499 | 0 | 0.001297 | 0.003889 | 0.003388 | 0.005177 | 0 | 0.001151 |
| rf26 | 0.002077 | 0.00648 | 0.001992 | 0.001829 | 0.005133 | 0.002422 | 0.002755 | 0.002848 | 0.009692 | 0.000926 |
| rf27 | 0 | 0 | 0.004836 | 0.002918 | 0.003922 | 0 | 0.000499 | 0.004864 | 0.001758 | 0.003741 |
| extraTrees0 | 0.003418 | 0.006157 | 0.002623 | 0.002986 | 0.010984 | 0.002572 | 0.00181 | 0.006535 | 0.003136 | 0.00163 |
| extraTrees1 | 0.002027 | 0.006215 | 0.00151 | 0.002327 | 0.003425 | 0.001565 | 0.009512 | 0.005188 | 0.003432 | 0.001084 |
| extraTrees2 | 0.002872 | 0.001536 | 0.002959 | 0.006053 | 0.007578 | 0.004816 | 0.004347 | 0.006481 | 0.006223 | 0.00164 |
| extraTrees3 | 0.001024 | 0.006342 | 0.004107 | 0.004319 | 0.00887 | 0.00226 | 0.000801 | 0.001279 | 0.007055 | 0.001917 |
| extraTrees4 | 0.003134 | 0.0055 | 0.001044 | 0.004283 | 0.002175 | 0.003418 | 0.003507 | 0.003167 | 0.007248 | 0.001083 |
| extraTrees5 | 0.001505 | 0.003869 | 0.000934 | 0.006381 | 0.003033 | 2.96E−05 | 0.006186 | 0.005662 | 0.002948 | 0.002005 |
| extraTrees6 | 0.003912 | 0.001244 | 0.002641 | 0.004726 | 0.005683 | 0.003025 | 0.00694 | 0.004093 | 0.003323 | 0.003265 |
| extraTrees7 | 0.004625 | 0.007998 | 0.003561 | 0.007 | 0.006285 | 0.001679 | 0.003753 | 0.002258 | 0.007885 | 0 |
| extraTrees8 | 0.002092 | 0.006472 | 0.004602 | 0.003917 | 0.006961 | 0.003025 | 0.004582 | 0.005788 | 0.004909 | 0.001945 |
| extraTrees9 | 0.003488 | 0.00321 | 0.003358 | 0.001896 | 0.006382 | 0.000912 | 0.00565 | 0.002157 | 0.006154 | 0.002537 |
| extraTrees10 | 0.003171 | 0.002157 | 0.004749 | 0.002843 | 0.006074 | 0.001535 | 6.66E−19 | 0.006745 | 0.003161 | 0.003288 |
| extraTrees11 | 0.000825 | 0.008219 | 0.000854 | 0.007375 | 0.005182 | 0.003166 | 0.000393 | 0.006192 | 0.008721 | 0.002973 |
| extraTrees12 | 0.002587 | 0.002117 | 0.00447 | 0.002557 | 0.007451 | 0.001555 | 0.002642 | 0.006891 | 0.00652 | 0.003989 |
| extraTrees13 | 0.005043 | 0.003442 | 0.003063 | 0.004232 | 0.002315 | 0.001865 | 0.000657 | 0.004959 | 0.005742 | 0.001831 |
| extraTrees14 | 0.003418 | 0.006157 | 0.002623 | 0.002986 | 0.010984 | 0.002572 | 0.00181 | 0.006535 | 0.003136 | 0.00163 |
| extraTrees15 | 0.002027 | 0.006215 | 0.00151 | 0.002327 | 0.003425 | 0.001565 | 0.009512 | 0.005188 | 0.003432 | 0.001084 |
| extraTrees16 | 0.002872 | 0.001536 | 0.002959 | 0.006053 | 0.007578 | 0.004816 | 0.004347 | 0.006481 | 0.006223 | 0.00164 |
| extraTrees17 | 0.001024 | 0.006342 | 0.004107 | 0.004319 | 0.00887 | 0.00226 | 0.000801 | 0.001279 | 0.007055 | 0.001917 |
| extraTrees18 | 0.003134 | 0.0055 | 0.001044 | 0.004283 | 0.002175 | 0.003418 | 0.003507 | 0.003167 | 0.007248 | 0.001083 |
| extraTrees19 | 0.001505 | 0.003869 | 0.000934 | 0.006381 | 0.003033 | 2.96E−05 | 0.006186 | 0.005662 | 0.002948 | 0.002005 |
| extraTrees20 | 0.003912 | 0.001244 | 0.002641 | 0.004726 | 0.005683 | 0.003025 | 0.00694 | 0.004093 | 0.003323 | 0.003265 |
| extraTrees21 | 0.004625 | 0.007998 | 0.003561 | 0.007 | 0.006285 | 0.001679 | 0.003753 | 0.002258 | 0.007885 | 0 |
| extraTrees22 | 0.002092 | 0.006472 | 0.004602 | 0.003917 | 0.006961 | 0.003025 | 0.004582 | 0.005788 | 0.004909 | 0.001945 |
| extraTrees23 | 0.003488 | 0.00321 | 0.003358 | 0.001896 | 0.006382 | 0.000912 | 0.00565 | 0.002157 | 0.006154 | 0.002537 |
| extraTrees24 | 0.003171 | 0.002157 | 0.004749 | 0.002843 | 0.006074 | 0.001535 | 6.66E−19 | 0.006745 | 0.003161 | 0.003288 |
| extraTrees25 | 0.000825 | 0.008219 | 0.000854 | 0.007375 | 0.005182 | 0.003166 | 0.000393 | 0.006192 | 0.008721 | 0.002973 |
| extraTrees26 | 0.002587 | 0.002117 | 0.00447 | 0.002557 | 0.007451 | 0.001555 | 0.002642 | 0.006891 | 0.00652 | 0.003989 |
| extraTrees27 | 0.005043 | 0.003442 | 0.003063 | 0.004232 | 0.002315 | 0.001865 | 0.000657 | 0.004959 | 0.005742 | 0.001831 |
| gbc0 | 0 | 0.000641 | 0 | 0.000109 | 0 | 0.015798 | 0.029819 | 0.000276 | 0.000232 | 0 |
| gbc1 | 0.014131 | 0.000214 | 3.09E−05 | 0.006005 | 0 | 0.000478 | 0.000255 | 0.006377 | 0.029347 | 0 |
| gbc2 | 0.000184 | 0 | 0.00256 | 0.003384 | 0 | 4.56E−10 | 0.000851 | 0.000613 | 0.045684 | 0.00053 |
| gbc3 | 0.000984 | 0.00548 | 0.000158 | 0.006124 | 0.023548 | 0 | 0.008275 | 0.000885 | 0 | 0 |
| gbc4 | 0.01011 | 2.15E−05 | 6.03E−05 | 0.000183 | 0 | 0.004588 | 0.00565 | 0.000561 | 2.30E−05 | 0 |
| gbc5 | 0.002695 | 0.002792 | 0.000289 | 0 | 0 | 8.58E−05 | 0.023742 | 0.013468 | 0.000176 | 0.004561 |
| gbc6 | 1.35E−05 | 0.007276 | 0 | 4.57E−05 | 7.28E−05 | 0.001854 | 0.001178 | 0.004045 | 0.000376 | 0 |
| gbc7 | 0.0058 | 0.000887 | 0.002149 | 0 | 0.00011 | 0.010789 | 0 | 0 | 0.000407 | 0 |
| gbc8 | 0.017161 | 0.00042 | 0.00894 | 0.000759 | 0 | 0 | 0.00366 | 0.00191 | 0.006164 | 0 |
| gbc9 | 0.023259 | 0.007344 | 0.001026 | 0 | 0.000307 | 0 | 0.001524 | 0.044679 | 0 | 0.002997 |
| gbc10 | 0.001936 | 0.015714 | 0.000851 | 6.37E−05 | 0 | 2.03E−05 | 0.010004 | 0.001845 | 0.000442 | 0.000379 |
| gbc11 | 0.000245 | 0.005068 | 0.002542 | 0.003061 | 0.000591 | 0.003382 | 0.00055 | 0.042308 | 0.010033 | 0.017932 |
| gbc12 | 8.40E−05 | 0.003025 | 0.000113 | 0.000321 | 0.007776 | 0.00224 | 0.002063 | 0.018504 | 0.000455 | 0.002502 |
| gbc13 | 0.000101 | 0 | 0.00017 | 0.002375 | 9.02E−05 | 0.000237 | 0.034059 | 0.012948 | 0.008072 | 0 |
| gbc14 | 0 | 0.000641 | 0 | 0.000109 | 0 | 0.015798 | 0.029819 | 0.000276 | 0.000232 | 0 |
| gbc15 | 0.014131 | 0.000214 | 3.09E−05 | 0.006005 | 0 | 0.000478 | 0.000255 | 0.006377 | 0.029347 | 0 |
| gbc16 | 0.000184 | 0 | 0.00256 | 0.003384 | 0 | 4.56E−10 | 0.000851 | 0.000613 | 0.045684 | 0.00053 |
| gbc17 | 0.000984 | 0.00548 | 0.000158 | 0.006124 | 0.023548 | 0 | 0.008275 | 0.000885 | 0 | 0 |
| gbc18 | 0.01011 | 2.15E−05 | 6.03E−05 | 0.000183 | 0 | 0.004588 | 0.00565 | 0.000561 | 2.30E−05 | 0 |
| gbc19 | 0.002695 | 0.002792 | 0.000289 | 0 | 0 | 8.58E−05 | 0.023742 | 0.013468 | 0.000176 | 0.004561 |
| gbc20 | 1.35E−05 | 0.007276 | 0 | 4.57E−05 | 7.28E−05 | 0.001854 | 0.001178 | 0.004045 | 0.000376 | 0 |
| gbc21 | 0.0058 | 0.000887 | 0.002149 | 0 | 0.00011 | 0.010789 | 0 | 0 | 0.000407 | 0 |
| gbc22 | 0.017161 | 0.00042 | 0.00894 | 0.000759 | 0 | 0 | 0.00366 | 0.00191 | 0.006164 | 0 |
| gbc23 | 0.023259 | 0.007344 | 0.001026 | 0 | 0.000307 | 0 | 0.001524 | 0.044679 | 0 | 0.002997 |
| gbc24 | 0.001936 | 0.015714 | 0.000851 | 6.37E−05 | 0 | 2.03E−05 | 0.010004 | 0.001845 | 0.000442 | 0.000379 |
| gbc25 | 0.000245 | 0.005068 | 0.002542 | 0.003061 | 0.000591 | 0.003382 | 0.00055 | 0.042308 | 0.010033 | 0.017932 |
| gbc26 | 8.40E−05 | 0.003025 | 0.000113 | 0.000321 | 0.007776 | 0.00224 | 0.002063 | 0.018504 | 0.000455 | 0.002502 |
| gbc27 | 0.000101 | 0 | 0.00017 | 0.002375 | 9.02E−05 | 0.000237 | 0.034059 | 0.012948 | 0.008072 | 0 |
| ada0 | 0.02 | 0 | 0 | 0.02 | 0 | 0.02 | 0.04 | 0 | 0.02 | 0 |
| ada1 | 0.02 | 0 | 0 | 0.02 | 0 | 0.02 | 0.04 | 0 | 0.02 | 0 |
| ada2 | 0.02 | 0 | 0 | 0.02 | 0 | 0.02 | 0.04 | 0 | 0.02 | 0 |
| ada3 | 0.02 | 0 | 0 | 0.02 | 0 | 0.02 | 0.04 | 0 | 0.02 | 0 |
| ada4 | 0.02 | 0 | 0 | 0.02 | 0 | 0.02 | 0.04 | 0 | 0.02 | 0 |
| ada5 | 0.02 | 0 | 0 | 0.02 | 0 | 0.02 | 0.04 | 0 | 0.02 | 0 |
| ada6 | 0.02 | 0 | 0 | 0.02 | 0 | 0.02 | 0.04 | 0 | 0.02 | 0 |
| ada7 | 0.02 | 0 | 0 | 0.02 | 0 | 0.02 | 0.04 | 0 | 0.02 | 0 |
| ada8 | 0.02 | 0 | 0 | 0.02 | 0 | 0.02 | 0.04 | 0 | 0.02 | 0 |
| ada9 | 0.02 | 0 | 0 | 0.02 | 0 | 0.02 | 0.04 | 0 | 0.02 | 0 |
| ada10 | 0.02 | 0 | 0 | 0.02 | 0 | 0.02 | 0.04 | 0 | 0.02 | 0 |
| ada11 | 0.02 | 0 | 0 | 0.02 | 0 | 0.02 | 0.04 | 0 | 0.02 | 0 |
| ada12 | 0.02 | 0 | 0 | 0.02 | 0 | 0.02 | 0.04 | 0 | 0.02 | 0 |
| ada13 | 0.02 | 0 | 0 | 0.02 | 0 | 0.02 | 0.04 | 0 | 0.02 | 0 |
| ada14 | 0.02 | 0 | 0 | 0.02 | 0 | 0.02 | 0.04 | 0 | 0.02 | 0 |
| ada15 | 0.02 | 0 | 0 | 0.02 | 0 | 0.02 | 0.04 | 0 | 0.02 | 0 |
| ada16 | 0.02 | 0 | 0 | 0.02 | 0 | 0.02 | 0.04 | 0 | 0.02 | 0 |
| ada17 | 0.02 | 0 | 0 | 0.02 | 0 | 0.02 | 0.04 | 0 | 0.02 | 0 |
| ada18 | 0.02 | 0 | 0 | 0.02 | 0 | 0.02 | 0.04 | 0 | 0.02 | 0 |
| ada19 | 0.02 | 0 | 0 | 0.02 | 0 | 0.02 | 0.04 | 0 | 0.02 | 0 |
| ada20 | 0.02 | 0 | 0 | 0.02 | 0 | 0.02 | 0.04 | 0 | 0.02 | 0 |
| ada21 | 0.02 | 0 | 0 | 0.02 | 0 | 0.02 | 0.04 | 0 | 0.02 | 0 |
| ada22 | 0.02 | 0 | 0 | 0.02 | 0 | 0.02 | 0.04 | 0 | 0.02 | 0 |
| ada23 | 0.02 | 0 | 0 | 0.02 | 0 | 0.02 | 0.04 | 0 | 0.02 | 0 |
| ada24 | 0.02 | 0 | 0 | 0.02 | 0 | 0.02 | 0.04 | 0 | 0.02 | 0 |
| ada25 | 0.02 | 0 | 0 | 0.02 | 0 | 0.02 | 0.04 | 0 | 0.02 | 0 |
| ada26 | 0.02 | 0 | 0 | 0.02 | 0 | 0.02 | 0.04 | 0 | 0.02 | 0 |
| ada27 | 0.02 | 0 | 0 | 0.02 | 0 | 0.02 | 0.04 | 0 | 0.02 | 0. |

| Gene | MGST2 | MICA | MYLK | MYO18B | NAP1L4 | NBEAP1 | NOMO2 | NR1D2 | PAPPA | PIP5K1B |
|---|---|---|---|---|---|---|---|---|---|---|
| rf0 | 0.004965 | 0 | 0 | 0.000985 | 0.000945 | 0.006443 | 0.002572 | 0.003094 | 0.006904 | 0.004322 |
| rf1 | 0.00467 | 0.001971 | 0.004031 | 0.0072 | 0.001696 | 0 | 0 | 0.002313 | 0.000444 | 0.003312 |
| rf2 | 0.0044 | 0.007093 | 0 | 0 | 0.003271 | 0.002706 | 0.005119 | 0.006285 | 0.002922 | 0 |
| rf3 | 0.00069 | 0.003527 | 0.003479 | 0 | 0.002727 | 0.002037 | 0.00449 | 0.007905 | 0.001246 | 0.001202 |
| rf4 | 0 | 0.001269 | 0.002296 | 0.002306 | 0.006964 | 0.004597 | 0.000311 | 0.011102 | 0.001422 | 0.004602 |
| rf5 | 0 | 0.007389 | 0.002483 | 0 | 0.002697 | 0.002224 | 0.001408 | 0.015057 | 0.000616 | 0.006764 |
| rf6 | 0.00037 | 0.003139 | 0.004051 | 0.000635 | 0.002072 | 0.00331 | 0.001517 | 0.004097 | 0.000311 | 0.007178 |
| rf7 | 6.86E−19 | 0.010417 | 0.002345 | 0.010415 | 0.001051 | 0.004239 | 0 | 0.013378 | 0.00646 | 0.003463 |
| rf8 | 0.002001 | 0.000711 | 0.003084 | 0.003637 | 0.005058 | 0.004066 | 0.000383 | 0.001027 | 0.002013 | 0.002226 |
| rf9 | 0.000428 | 0.000702 | 0.002168 | 0.000384 | 0 | 0.013052 | 0.002465 | 0.004351 | 0.003288 | 0.003607 |
| rf10 | 0.004475 | 0 | 0 | 0.001139 | 0 | 0 | 0.005679 | 0.000344 | 0.010106 | 0.004427 |
| rf11 | 0 | 0.001538 | 0.00277 | 0.002376 | 0.007374 | 0.017909 | 0.00063 | 0.004573 | 0.001087 | 0.002783 |
| rf12 | 0.005179 | 0 | 0.002027 | 0.000633 | 0.001303 | 0.001743 | 0.001037 | 0.004762 | 0.004156 | 0.001697 |
| rf13 | 0 | 0.001157 | 0 | 0.001642 | 0.010385 | 0.007259 | 0.004978 | 0.00157 | 0 | 0.000734 |
| rf14 | 0.004965 | 0 | 0 | 0.000985 | 0.000945 | 0.006443 | 0.002572 | 0.003094 | 0.006904 | 0.004322 |
| rf15 | 0.00467 | 0.001971 | 0.004031 | 0.0072 | 0.001696 | 0 | 0 | 0.002313 | 0.000444 | 0.003312 |
| rf16 | 0.0044 | 0.007093 | 0 | 0 | 0.003271 | 0.002706 | 0.005119 | 0.006285 | 0.002922 | 0 |
| rf17 | 0.00069 | 0.003527 | 0.003479 | 0 | 0.002727 | 0.002037 | 0.00449 | 0.007905 | 0.001246 | 0.001202 |
| rf18 | 0 | 0.001269 | 0.002296 | 0.002306 | 0.006964 | 0.004597 | 0.000311 | 0.011102 | 0.001422 | 0.004602 |
| rf19 | 0 | 0.007389 | 0.002483 | 0 | 0.002697 | 0.002224 | 0.001408 | 0.015057 | 0.000616 | 0.006764 |
| rf20 | 0.00037 | 0.003139 | 0.004051 | 0.000635 | 0.002072 | 0.00331 | 0.001517 | 0.004097 | 0.000311 | 0.007178 |
| rf21 | 6.86E−19 | 0.010417 | 0.002345 | 0.010415 | 0.001051 | 0.004239 | 0 | 0.013378 | 0.00646 | 0.003463 |
| rf22 | 0.002001 | 0.000711 | 0.003084 | 0.003637 | 0.005058 | 0.004066 | 0.000383 | 0.001027 | 0.002013 | 0.002226 |
| rf23 | 0.000428 | 0.000702 | 0.002168 | 0.000384 | 0 | 0.013052 | 0.002465 | 0.004351 | 0.003288 | 0.003607 |
| rf24 | 0.004475 | 0 | 0 | 0.001139 | 0 | 0 | 0.005679 | 0.000344 | 0.010106 | 0.004427 |
| rf25 | 0 | 0.001538 | 0.00277 | 0.002376 | 0.007374 | 0.017909 | 0.00063 | 0.004573 | 0.001087 | 0.002783 |
| rf26 | 0.005179 | 0 | 0.002027 | 0.000633 | 0.001303 | 0.001743 | 0.001037 | 0.004762 | 0.004156 | 0.001697 |
| rf27 | 0 | 0.001157 | 0 | 0.001642 | 0.010385 | 0.007259 | 0.004978 | 0.00157 | 0 | 0.000734 |
| extraTrees0 | 0.00456 | 0.002621 | 0.001922 | 0.004284 | 0.002699 | 0.002293 | 0.001779 | 0.002433 | 0.005006 | 0.003709 |
| extraTrees1 | 0.002149 | 0.000862 | 0.003605 | 0.004121 | 0.002792 | 0.00364 | 0.001501 | 0.007978 | 0.000685 | 0.001519 |
| extraTrees2 | 0.003324 | 0.001149 | 0.002018 | 0.001502 | 0.002778 | 0.0075 | 0.003006 | 0.011885 | 0.00162 | 0.001224 |
| extraTrees3 | 0.001604 | 0.0048 | 0.003549 | 6.61E−18 | 0.001735 | 0.003075 | 0.000712 | 0.005219 | 0.003757 | 0.003278 |
| extraTrees4 | 0.004881 | 0.001425 | 0.002811 | 0.000625 | 0.006391 | 0.004715 | 0.00391 | 0.005139 | 0.000199 | 0.000552 |
| extraTrees5 | 0.003651 | 0.001058 | 0.000388 | 0.001643 | 0.000699 | 0.003263 | 0.000744 | 0.00587 | 0.002004 | 0.002624 |
| extraTrees6 | 0.003774 | 0.001782 | 0.004043 | 0.001693 | 0.003917 | 0.001104 | 0.002127 | 0.005907 | 0.002915 | 0.001889 |
| extraTrees7 | 0.004133 | 0.003199 | 0.001892 | 0.001669 | 0.005326 | 0.00756 | 0.001227 | 0.006508 | 0.003953 | 0.002979 |
| extraTrees8 | 0.004096 | 0.000512 | 0.006413 | 0.003672 | 0.0039 | 0.004358 | 0.001965 | 0.002722 | 0.000503 | 0.001894 |
| extraTrees9 | 0.001131 | 0.004614 | 0.001132 | 0.002005 | 0.003388 | 0.004739 | 0.00092 | 0.004961 | 0.002165 | 0.004829 |
| extraTrees10 | 0.001877 | 0.00349 | 0.000794 | 0.001838 | 0.005394 | 0.005677 | 0.001923 | 0.005395 | 0.001885 | 0.002828 |
| extraTrees11 | 0.002632 | 0.002946 | 0.002259 | 0.001503 | 0.003882 | 0.005126 | 0.000862 | 0.010065 | 0.000589 | 0.001228 |
| extraTrees12 | 0.005278 | 0.004704 | 0.001849 | 0.00252 | 0.001219 | 0.002544 | 0.000515 | 0.004995 | 0.001094 | 0.003408 |
| extraTrees13 | 0.003728 | 0.000677 | 0.005775 | 0.003197 | 0.006684 | 0.001755 | 0.003432 | 0.003164 | 0.003572 | 0.008665 |
| extraTrees14 | 0.00456 | 0.002621 | 0.001922 | 0.004284 | 0.002699 | 0.002293 | 0.001779 | 0.002433 | 0.005006 | 0.003709 |
| extraTrees15 | 0.002149 | 0.000862 | 0.003605 | 0.004121 | 0.002792 | 0.00364 | 0.001501 | 0.007978 | 0.000685 | 0.001519 |
| extraTrees16 | 0.003324 | 0.001149 | 0.002018 | 0.001502 | 0.002778 | 0.0075 | 0.003006 | 0.011885 | 0.00162 | 0.001224 |
| extraTrees17 | 0.001604 | 0.0048 | 0.003549 | 6.61E−18 | 0.001735 | 0.003075 | 0.000712 | 0.005219 | 0.003757 | 0.003278 |
| extraTrees18 | 0.004881 | 0.001425 | 0.002811 | 0.000625 | 0.006391 | 0.004715 | 0.00391 | 0.005139 | 0.000199 | 0.000552 |
| extraTrees19 | 0.003651 | 0.001058 | 0.000388 | 0.001643 | 0.000699 | 0.003263 | 0.000744 | 0.00587 | 0.002004 | 0.002624 |
| extraTrees20 | 0.003774 | 0.001782 | 0.004043 | 0.001693 | 0.003917 | 0.001104 | 0.002127 | 0.005907 | 0.002915 | 0.001889 |
| extraTrees21 | 0.004133 | 0.003199 | 0.001892 | 0.001669 | 0.005326 | 0.00756 | 0.001227 | 0.006508 | 0.003953 | 0.002979 |
| extraTrees22 | 0.004096 | 0.000512 | 0.006413 | 0.003672 | 0.0039 | 0.004358 | 0.001965 | 0.002722 | 0.000503 | 0.001894 |
| extraTrees23 | 0.001131 | 0.004614 | 0.001132 | 0.002005 | 0.003388 | 0.004739 | 0.00092 | 0.004961 | 0.002165 | 0.004829 |
| extraTrees24 | 0.001877 | 0.00349 | 0.000794 | 0.001838 | 0.005394 | 0.005677 | 0.001923 | 0.005395 | 0.001885 | 0.002828 |
| extraTrees25 | 0.002632 | 0.002946 | 0.002259 | 0.001503 | 0.003882 | 0.005126 | 0.000862 | 0.010065 | 0.000589 | 0.001228 |
| extraTrees26 | 0.005278 | 0.004704 | 0.001849 | 0.00252 | 0.001219 | 0.002544 | 0.000515 | 0.004995 | 0.001094 | 0.003408 |
| extraTrees27 | 0.003728 | 0.000677 | 0.005775 | 0.003197 | 0.006684 | 0.001755 | 0.003432 | 0.003164 | 0.003572 | 0.008665 |
| gbc0 | 0 | 3.28E−05 | 7.34E−05 | 0.001969 | 0.004198 | 0.004823 | 0.002165 | 0.017146 | 9.37E−05 | 0.00913 |
| gbc1 | 9.39E−05 | 0.000327 | 0 | 0.005441 | 0 | 0.00021 | 0.000544 | 0.000182 | 0 | 1.36E−05 |
| gbc2 | 0.00484 | 0.026324 | 0.004098 | 0 | 0 | 0.0038 | 0.000141 | 0.002155 | 0 | 0.000206 |
| gbc3 | 0.000342 | 0.004754 | 0.000432 | 0.004905 | 0 | 0.001978 | 8.68E−08 | 6.96E−05 | 5.06E−05 | 0 |
| gbc4 | 0.00673 | 0.000538 | 0.016026 | 0.00124 | 0.000307 | 0.041374 | 0 | 0.010863 | 0.00043 | 0.006961 |
| gbc5 | 0.00356 | 0.003146 | 0.001017 | 0.000514 | 2.59E−05 | 0.002031 | 0.005037 | 0.005102 | 0.000828 | 0.000559 |
| gbc6 | 0.002782 | 0.003047 | 3.96E−05 | 0.00034 | 0 | 0.004567 | 1.54E−05 | 0.000599 | 0 | 0.001084 |
| gbc7 | 0 | 0 | 0.000367 | 0.002344 | 0.000107 | 0.018265 | 0.034925 | 0 | 0.001046 | 0.024441 |
| gbc8 | 0 | 0.012237 | 0.000329 | 0.003263 | 0 | 0.000609 | 0 | 0 | 0.003479 | 0.002721 |
| gbc9 | 0 | 0.022964 | 0 | 2.20E−05 | 3.82E−05 | 0.015877 | 0 | 0 | 0 | 0.002276 |
| gbc10 | 0.005945 | 0.007027 | 0.000755 | 0.000367 | 9.28E−05 | 0.005467 | 4.36E−05 | 0.00104 | 0.005067 | 0.015802 |
| gbc11 | 0.00014 | 0.00137 | 0 | 0.000104 | 3.66E−05 | 0.002538 | 0.003588 | 0.004706 | 8.40E−05 | 0 |
| gbc12 | 4.94E−05 | 0.00577 | 0 | 0 | 0.001587 | 0.024821 | 0.004583 | 0.011032 | 0.01663 | 0.000191 |
| gbc13 | 0 | 0.013501 | 0 | 3.19E−05 | 0 | 0.009964 | 0.009181 | 3.46E−05 | 0 | 3.14E−05 |
| gbc14 | 0 | 3.28E−05 | 7.34E−05 | 0.001969 | 0.004198 | 0.004823 | 0.002165 | 0.017146 | 9.37E−05 | 0.00913 |
| gbc15 | 9.39E−05 | 0.000327 | 0 | 0.005441 | 0 | 0.00021 | 0.000544 | 0.000182 | 0 | 1.36E−05 |
| gbc16 | 0.00484 | 0.026324 | 0.004098 | 0 | 0 | 0.0038 | 0.000141 | 0.002155 | 0 | 0.000206 |
| gbc17 | 0.000342 | 0.004754 | 0.000432 | 0.004905 | 0 | 0.001978 | 8.68E−08 | 6.96E−05 | 5.06E−05 | 0 |
| gbc18 | 0.00673 | 0.000538 | 0.016026 | 0.00124 | 0.000307 | 0.041374 | 0 | 0.010863 | 0.00043 | 0.006961 |
| gbc19 | 0.00356 | 0.003146 | 0.001017 | 0.000514 | 2.59E−05 | 0.002031 | 0.005037 | 0.005102 | 0.000828 | 0.000559 |
| gbc20 | 0.002782 | 0.003047 | 3.96E−05 | 0.00034 | 0 | 0.004567 | 1.54E−05 | 0.000599 | 0 | 0.001084 |
| gbc21 | 0 | 0 | 0.000367 | 0.002344 | 0.000107 | 0.018265 | 0.034925 | 0 | 0.001046 | 0.024441 |
| gbc22 | 0 | 0.012237 | 0.000329 | 0.003263 | 0 | 0.000609 | 0 | 0 | 0.003479 | 0.002721 |
| gbc23 | 0 | 0.022964 | 0 | 2.20E−05 | 3.82E−05 | 0.015877 | 0 | 0 | 0 | 0.002276 |
| gbc24 | 0.005945 | 0.007027 | 0.000755 | 0.000367 | 9.28E−05 | 0.005467 | 4.36E−05 | 0.00104 | 0.005067 | 0.015802 |
| gbc25 | 0.00014 | 0.00137 | 0 | 0.000104 | 3.66E−05 | 0.002538 | 0.003588 | 0.004706 | 8.40E−05 | 0 |
| gbc26 | 4.94E−05 | 0.00577 | 0 | 0 | 0.001587 | 0.024821 | 0.004583 | 0.011032 | 0.01663 | 0.000191 |
| gbc27 | 0 | 0.013501 | 0 | 3.19E−05 | 0 | 0.009964 | 0.009181 | 3.46E−05 | 0 | 3.14E−05 |
| ada0 | 0 | 0.02 | 0 | 0 | 0 | 0.02 | 0 | 0.02 | 0 | 0 |
| ada1 | 0 | 0.02 | 0 | 0 | 0 | 0.02 | 0 | 0.02 | 0 | 0 |
| ada2 | 0 | 0.02 | 0 | 0 | 0 | 0.02 | 0 | 0.02 | 0 | 0 |
| ada3 | 0 | 0.02 | 0 | 0 | 0 | 0.02 | 0 | 0.02 | 0 | 0 |
| ada4 | 0 | 0.02 | 0 | 0 | 0 | 0.02 | 0 | 0.02 | 0 | 0 |
| ada5 | 0 | 0.02 | 0 | 0 | 0 | 0.02 | 0 | 0.02 | 0 | 0 |
| ada6 | 0 | 0.02 | 0 | 0 | 0 | 0.02 | 0 | 0.02 | 0 | 0 |
| ada7 | 0 | 0.02 | 0 | 0 | 0 | 0.02 | 0 | 0.02 | 0 | 0 |
| ada8 | 0 | 0.02 | 0 | 0 | 0 | 0.02 | 0 | 0.02 | 0 | 0 |
| ada9 | 0 | 0.02 | 0 | 0 | 0 | 0.02 | 0 | 0.02 | 0 | 0 |
| ada10 | 0 | 0.02 | 0 | 0 | 0 | 0.02 | 0 | 0.02 | 0 | 0 |
| ada11 | 0 | 0.02 | 0 | 0 | 0 | 0.02 | 0 | 0.02 | 0 | 0 |
| ada12 | 0 | 0.02 | 0 | 0 | 0 | 0.02 | 0 | 0.02 | 0 | 0 |
| ada13 | 0 | 0.02 | 0 | 0 | 0 | 0.02 | 0 | 0.02 | 0 | 0 |
| ada14 | 0 | 0.02 | 0 | 0 | 0 | 0.02 | 0 | 0.02 | 0 | 0 |
| ada15 | 0 | 0.02 | 0 | 0 | 0 | 0.02 | 0 | 0.02 | 0 | 0 |
| ada16 | 0 | 0.02 | 0 | 0 | 0 | 0.02 | 0 | 0.02 | 0 | 0 |
| ada17 | 0 | 0.02 | 0 | 0 | 0 | 0.02 | 0 | 0.02 | 0 | 0 |
| ada18 | 0 | 0.02 | 0 | 0 | 0 | 0.02 | 0 | 0.02 | 0 | 0 |
| ada19 | 0 | 0.02 | 0 | 0 | 0 | 0.02 | 0 | 0.02 | 0 | 0 |
| ada20 | 0 | 0.02 | 0 | 0 | 0 | 0.02 | 0 | 0.02 | 0 | 0 |
| ada21 | 0 | 0.02 | 0 | 0 | 0 | 0.02 | 0 | 0.02 | 0 | 0 |
| ada22 | 0 | 0.02 | 0 | 0 | 0 | 0.02 | 0 | 0.02 | 0 | 0 |
| ada23 | 0 | 0.02 | 0 | 0 | 0 | 0.02 | 0 | 0.02 | 0 | 0 |
| ada24 | 0 | 0.02 | 0 | 0 | 0 | 0.02 | 0 | 0.02 | 0 | 0 |
| ada25 | 0 | 0.02 | 0 | 0 | 0 | 0.02 | 0 | 0.02 | 0 | 0 |
| ada26 | 0 | 0.02 | 0 | 0 | 0 | 0.02 | 0 | 0.02 | 0 | 0 |
| ada27 | 0 | 0.02 | 0 | 0 | 0 | 0.02 | 0 | 0.02 | 0 | 0. |

| Gene | PTMAP10 | RAB3GAP2 | RAC1P4 | RAD51AP1 | RASA1 | RTN1 | SEC22A | SEM1 | SEPT7P7 | SLC3A2 |
|---|---|---|---|---|---|---|---|---|---|---|
| rf0 | 0.009213 | 0.005311 | 0.005753 | 0.000342 | 0.002233 | 0.007024 | 0.002026 | 0 | 0.002179 | 0.003008 |
| rf1 | 0.010114 | 0.000404 | 0.000387 | 0.00441 | 0 | 0 | 0.001993 | 0 | 0 | 0.003725 |
| rf2 | 0.004811 | 0.001316 | 0 | 0.008138 | 0.001011 | 0.003057 | 0.008121 | 0.006067 | 4.70E−19 | 0 |
| rf3 | 0.001601 | 3.77E−18 | 0.00052 | 0.000679 | 0.003196 | 0.007042 | 0 | 0.002204 | 0.00412 | 0.00065 |
| rf4 | 0.003888 | 0.001705 | 0.001353 | 0.002075 | 0 | 0.007855 | 0.000726 | 0.001164 | 0.001023 | 0.002636 |
| rf5 | 0.00241 | 0.003847 | 0 | 0.014339 | 0 | 0.007459 | 0.004785 | 0.00293 | 0.000352 | 0.002671 |
| rf6 | 4.27E−18 | 0.000882 | 0.002146 | 0.00609 | 0 | 3.98E−05 | 0 | 0 | 0.000329 | 0.00166 |
| rf7 | 0.000999 | 0.001187 | 0 | 0.00441 | 0.000368 | 0.003699 | 0.003941 | 0.002271 | 0 | 0.001679 |
| rf8 | 0.002973 | 0.000796 | 0 | 0.00123 | 0 | 0 | 0.002634 | 0.000712 | 0.001731 | 0 |
| rf9 | 0.005895 | 0.005913 | 0 | 0.004788 | 0.001445 | 0.006591 | 0.008202 | 0.000971 | 0.001072 | 0 |
| rf10 | 0.004567 | 0.003938 | 0.00081 | 0.00769 | 0.002194 | 0.002859 | 0.005693 | 0.000773 | 0.001342 | 0.002553 |
| rf11 | 0.000393 | 0.000574 | 0 | 0.005885 | 0.001417 | 0.009477 | 0.009525 | 8.97E−19 | 0.004243 | 0.002896 |
| rf12 | 0.003256 | 0.002012 | 0.000477 | 0.007442 | 0.000413 | 0.001174 | 0.005122 | 0.000911 | 0.004556 | 0.003142 |
| rf13 | 0.005757 | 6.91E−18 | 0.000366 | 0.001405 | 0 | 0 | 0 | 0.004858 | 0.006145 | 0.001625 |
| rf14 | 0.009213 | 0.005311 | 0.005753 | 0.000342 | 0.002233 | 0.007024 | 0.002026 | 0 | 0.002179 | 0.003008 |
| rf15 | 0.010114 | 0.000404 | 0.000387 | 0.00441 | 0 | 0 | 0.001993 | 0 | 0 | 0.003725 |
| rf16 | 0.004811 | 0.001316 | 0 | 0.008138 | 0.001011 | 0.003057 | 0.008121 | 0.006067 | 4.70E−19 | 0 |
| rf17 | 0.001601 | 3.77E−18 | 0.00052 | 0.000679 | 0.003196 | 0.007042 | 0 | 0.002204 | 0.00412 | 0.00065 |
| rf18 | 0.003888 | 0.001705 | 0.001353 | 0.002075 | 0 | 0.007855 | 0.000726 | 0.001164 | 0.001023 | 0.002636 |
| rf19 | 0.00241 | 0.003847 | 0 | 0.014339 | 0 | 0.007459 | 0.004785 | 0.00293 | 0.000352 | 0.002671 |
| rf20 | 4.27E−18 | 0.000882 | 0.002146 | 0.00609 | 0 | 3.98E−05 | 0 | 0 | 0.000329 | 0.00166 |
| rf21 | 0.000999 | 0.001187 | 0 | 0.00441 | 0.000368 | 0.003699 | 0.003941 | 0.002271 | 0 | 0.001679 |
| rf22 | 0.002973 | 0.000796 | 0 | 0.00123 | 0 | 0 | 0.002634 | 0.000712 | 0.001731 | 0 |
| rf23 | 0.005895 | 0.005913 | 0 | 0.004788 | 0.001445 | 0.006591 | 0.008202 | 0.000971 | 0.001072 | 0 |
| rf24 | 0.004567 | 0.003938 | 0.00081 | 0.00769 | 0.002194 | 0.002859 | 0.005693 | 0.000773 | 0.001342 | 0.002553 |
| rf25 | 0.000393 | 0.000574 | 0 | 0.005885 | 0.001417 | 0.009477 | 0.009525 | 8.97E−19 | 0.004243 | 0.002896 |
| rf26 | 0.003256 | 0.002012 | 0.000477 | 0.007442 | 0.000413 | 0.001174 | 0.005122 | 0.000911 | 0.004556 | 0.003142 |
| rf27 | 0.005757 | 6.91E−18 | 0.000366 | 0.001405 | 0 | 0 | 0 | 0.004858 | 0.006145 | 0.001625 |
| extraTrees0 | 0.008454 | 0.002258 | 0.001681 | 0.002274 | 2.73E−19 | 0.002679 | 0.002054 | 0.004841 | 0.002396 | 0.005452 |
| extraTrees1 | 0.004516 | 0.002774 | 0.001029 | 0.006556 | 0.002606 | 0.001893 | 0.003345 | 0.000799 | 0.001282 | 0.001294 |
| extraTrees2 | 0.004586 | 0 | 0.00236 | 0.008875 | 0.001059 | 0.002551 | 0.002539 | 0 | 0.001699 | 0.000932 |
| extraTrees3 | 0.006667 | 0.000575 | 0.00249 | 0.004436 | 0.002431 | 0.003 | 0.001002 | 0.000374 | 0.001664 | 0 |
| extraTrees4 | 0.007484 | 0.002585 | 0.003502 | 0.010595 | 0.000939 | 0.005421 | 0.004 | 0.001003 | 0.002067 | 0.002701 |
| extraTrees5 | 0.002228 | 0.002218 | 0.001331 | 0.013741 | 0.002335 | 0.00112 | 0.005192 | 0.000476 | 0.001006 | 0.001127 |
| extraTrees6 | 0.003898 | 0.003435 | 0.002382 | 0.013212 | 0.000388 | 0.002935 | 0.005222 | 0.001464 | 0.002621 | 0.000673 |
| extraTrees7 | 0.007833 | 0.001791 | 0.004556 | 0.006603 | 0.001737 | 0.004341 | 0.004634 | 0 | 0.003824 | 0.00097 |
| extraTrees8 | 0.004824 | 0.001084 | 0.003606 | 0.002999 | 0.0034 | 0.005419 | 0.000769 | 0.00034 | 0.001982 | 0.004095 |
| extraTrees9 | 0.010055 | 0.002269 | 0.002347 | 0.005403 | 0.001889 | 0.004467 | 0.001887 | 0.001569 | 0.001773 | 0 |
| extraTrees10 | 0.006128 | 0.000419 | 0.002048 | 0.004585 | 0.003155 | 0.001722 | 0.002868 | 0.001698 | 0.003001 | 0.000739 |
| extraTrees11 | 0.002243 | 0.003581 | 0.002661 | 0.003598 | 1.26E−18 | 0.002813 | 0.004448 | 0.005384 | 0.000268 | 0.000848 |
| extraTrees12 | 0.004843 | 0.002465 | 0.002069 | 0.007867 | 0.00206 | 0.000369 | 0.003907 | 0.000489 | 0.001388 | 0.001955 |
| extraTrees13 | 0.00402 | 0.001887 | 0.002447 | 0.008197 | 0.000327 | 0.006457 | 0.002431 | 0.002775 | 0.001788 | 0.000803 |
| extraTrees14 | 0.008454 | 0.002258 | 0.001681 | 0.002274 | 2.73E−19 | 0.002679 | 0.002054 | 0.004841 | 0.002396 | 0.005452 |
| extraTrees15 | 0.004516 | 0.002774 | 0.001029 | 0.006556 | 0.002606 | 0.001893 | 0.003345 | 0.000799 | 0.001282 | 0.001294 |
| extraTrees16 | 0.004586 | 0 | 0.00236 | 0.008875 | 0.001059 | 0.002551 | 0.002539 | 0 | 0.001699 | 0.000932 |
| extraTrees17 | 0.006667 | 0.000575 | 0.00249 | 0.004436 | 0.002431 | 0.003 | 0.001002 | 0.000374 | 0.001664 | 0 |
| extraTrees18 | 0.007484 | 0.002585 | 0.003502 | 0.010595 | 0.000939 | 0.005421 | 0.004 | 0.001003 | 0.002067 | 0.002701 |
| extraTrees19 | 0.002228 | 0.002218 | 0.001331 | 0.013741 | 0.002335 | 0.00112 | 0.005192 | 0.000476 | 0.001006 | 0.001127 |
| extraTrees20 | 0.003898 | 0.003435 | 0.002382 | 0.013212 | 0.000388 | 0.002935 | 0.005222 | 0.001464 | 0.002621 | 0.000673 |
| extraTrees21 | 0.007833 | 0.001791 | 0.004556 | 0.006603 | 0.001737 | 0.004341 | 0.004634 | 0 | 0.003824 | 0.00097 |
| extraTrees22 | 0.004824 | 0.001084 | 0.003606 | 0.002999 | 0.0034 | 0.005419 | 0.000769 | 0.00034 | 0.001982 | 0.004095 |
| extraTrees23 | 0.010055 | 0.002269 | 0.002347 | 0.005403 | 0.001889 | 0.004467 | 0.001887 | 0.001569 | 0.001773 | 0 |
| extraTrees24 | 0.006128 | 0.000419 | 0.002048 | 0.004585 | 0.003155 | 0.001722 | 0.002868 | 0.001698 | 0.003001 | 0.000739 |
| extraTrees25 | 0.002243 | 0.003581 | 0.002661 | 0.003598 | 1.26E−18 | 0.002813 | 0.004448 | 0.005384 | 0.000268 | 0.000848 |
| extraTrees26 | 0.004843 | 0.002465 | 0.002069 | 0.007867 | 0.00206 | 0.000369 | 0.003907 | 0.000489 | 0.001388 | 0.001955 |
| extraTrees27 | 0.00402 | 0.001887 | 0.002447 | 0.008197 | 0.000327 | 0.006457 | 0.002431 | 0.002775 | 0.001788 | 0.000803 |
| gbc0 | 0.000397 | 0.005563 | 0 | 0.000479 | 0 | 0.000287 | 0.000384 | 2.41E−06 | 2.35E−05 | 0.034601 |
| gbc1 | 0.015584 | 0.005318 | 0.000107 | 0 | 0 | 0 | 0.001803 | 0 | 0.001513 | 0.002539 |
| gbc2 | 1.01E−06 | 7.12E−05 | 5.59E−05 | 0.00081 | 0.000624 | 0 | 0 | 2.19E−05 | 0.00046 | 0.000598 |
| gbc3 | 0.004094 | 0 | 6.41E−05 | 0 | 0.011657 | 0 | 0.002154 | 5.53E−05 | 0 | 0.002664 |
| gbc4 | 0.000516 | 1.44E−05 | 0 | 0.008057 | 0 | 0 | 0.001517 | 0.000974 | 0 | 0.000836 |
| gbc5 | 0.003292 | 0.00046 | 0 | 0 | 0.005847 | 0.000861 | 0 | 0 | 0.005427 | 0.000749 |
| gbc6 | 0 | 0.008987 | 0.000196 | 0 | 0 | 0.003268 | 0.003638 | 0.000299 | 0 | 0.009371 |
| gbc7 | 0.001058 | 0.006363 | 0.000184 | 0 | 3.11E−05 | 0.006508 | 0.001031 | 7.02E−05 | 0.002361 | 0.009826 |
| gbc8 | 0 | 0.00032 | 0 | 0 | 0 | 3.12E−06 | 0.000568 | 0 | 0 | 0.010879 |
| gbc9 | 0.046845 | 0.000436 | 0 | 0 | 1.29E−05 | 0.018234 | 0 | 0 | 0.000348 | 0.00028 |
| gbc10 | 0.019073 | 0.00115 | 0 | 1.76E−05 | 0 | 0.00018 | 0.007198 | 0 | 0.000203 | 0 |
| gbc11 | 0.002292 | 0 | 0 | 0 | 0 | 0.013804 | 2.32E−06 | 0.000157 | 0 | 0 |
| gbc12 | 0 | 0 | 4.77E−05 | 0.014062 | 0 | 0.016061 | 0 | 5.45E−05 | 0 | 0 |
| gbc13 | 0 | 0.003758 | 0.008321 | 0.000285 | 0 | 0.003757 | 0.000101 | 0.00037 | 7.78E−06 | 0.026825 |
| gbc14 | 0.000397 | 0.005563 | 0 | 0.000479 | 0 | 0.000287 | 0.000384 | 2.41E−06 | 2.35E−05 | 0.034601 |
| gbc15 | 0.015584 | 0.005318 | 0.000107 | 0 | 0 | 0 | 0.001803 | 0 | 0.001513 | 0.002539 |
| gbc16 | 1.01E−06 | 7.12E−05 | 5.59E−05 | 0.00081 | 0.000624 | 0 | 0 | 2.19E−05 | 0.00046 | 0.000598 |
| gbc17 | 0.004094 | 0 | 6.41E−05 | 0 | 0.011657 | 0 | 0.002154 | 5.53E−05 | 0 | 0.002664 |
| gbc18 | 0.000516 | 1.44E−05 | 0 | 0.008057 | 0 | 0 | 0.001517 | 0.000974 | 0 | 0.000836 |
| gbc19 | 0.003292 | 0.00046 | 0 | 0 | 0.005847 | 0.000861 | 0 | 0 | 0.005427 | 0.000749 |
| gbc20 | 0 | 0.008987 | 0.000196 | 0 | 0 | 0.003268 | 0.003638 | 0.000299 | 0 | 0.009371 |
| gbc21 | 0.001058 | 0.006363 | 0.000184 | 0 | 3.11E−05 | 0.006508 | 0.001031 | 7.02E−05 | 0.002361 | 0.009826 |
| gbc22 | 0 | 0.00032 | 0 | 0 | 0 | 3.12E−06 | 0.000568 | 0 | 0 | 0.010879 |
| gbc23 | 0.046845 | 0.000436 | 0 | 0 | 1.29E−05 | 0.018234 | 0 | 0 | 0.000348 | 0.00028 |
| gbc24 | 0.019073 | 0.00115 | 0 | 1.76E−05 | 0 | 0.00018 | 0.007198 | 0 | 0.000203 | 0 |
| gbc25 | 0.002292 | 0 | 0 | 0 | 0 | 0.013804 | 2.32E−06 | 0.000157 | 0 | 0 |
| gbc26 | 0 | 0 | 4.77E−05 | 0.014062 | 0 | 0.016061 | 0 | 5.45E−05 | 0 | 0 |
| gbc27 | 0 | 0.003758 | 0.008321 | 0.000285 | 0 | 0.003757 | 0.000101 | 0.00037 | 7.78E−06 | 0.026825 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0. |

| Gene | SPARC | SPIN4 | SUGT1P1 | SVIL | TCAIM | THNSL1 | THUMPD2 | TRBV4-1 | TSPAN13 | UBE4B |
|---|---|---|---|---|---|---|---|---|---|---|
| rf0 | 0.000818 | 0.000455 | 0 | 0.00361 | 0.001133 | 0.00552 | 0.002769 | 0.003728 | 0.00424 | 0.001461 |
| rf1 | 0.000281 | 0.000946 | 0.001035 | 0 | 0.001519 | 0.003394 | 0.000892 | 0.002335 | 0.002714 | 0.000298 |
| rf2 | 0.001148 | 0.000647 | 0 | 0 | 0 | 0.001888 | 0.003263 | 0.001993 | 0.007462 | 0.000551 |
| rf3 | 0.004242 | 0.005207 | 0.007909 | 0 | 0.000394 | 0.004396 | 0.002692 | 0 | 0.000349 | 0.001744 |
| rf4 | 0 | 0.000365 | 0.005033 | 0.005812 | 0.005267 | 0.001339 | 0.003366 | 0.003825 | 0.003566 | 0.001282 |
| rf5 | 0.001463 | 0.001157 | 0.000834 | 0.002887 | 0.001473 | 0.003016 | 0.001762 | 0.001456 | 0.00262 | 0.002579 |
| rf6 | 0.00035 | 0 | 0.000378 | 0.002398 | 0.001289 | 0.005888 | 0.004065 | 0.004011 | 0.004141 | 0.006701 |
| rf7 | 0.003495 | 0 | 0.000559 | 0.001171 | 0 | 0.006693 | 0.003648 | 0.002405 | 0.002092 | 0 |
| rf8 | 0 | 0.002321 | 0.001299 | 0.002767 | 0.001892 | 0.003027 | 7.77E−05 | 0.00584 | 0.001206 | 0.002339 |
| rf9 | 0.003048 | 0.007102 | 0 | 0 | 0.002513 | 0.004599 | 0.00151 | 0.006174 | 0.002441 | 0 |
| rf10 | 0.00069 | 0.01181 | 0 | 0 | 0.000288 | 0.00652 | 0.00221 | 0.001317 | 0.009443 | 0.004121 |
| rf11 | 0.00211 | 0 | 0.001841 | 0.000225 | 0.00746 | 0.003257 | 0.004587 | 0.000374 | 0.002453 | 3.18E−19 |
| rf12 | 0.002828 | 0.001188 | 0.002249 | 0 | 0 | 4.14E−05 | 0.004817 | 0.008764 | 0.009296 | 0.000763 |
| rf13 | 0.00443 | 0.007098 | 0.000925 | 0 | 0.006391 | 0.004752 | 0.003646 | 0.001479 | 0.001448 | 0 |
| rf14 | 0.000818 | 0.000455 | 0 | 0.00361 | 0.001133 | 0.00552 | 0.002769 | 0.003728 | 0.00424 | 0.001461 |
| rf15 | 0.000281 | 0.000946 | 0.001035 | 0 | 0.001519 | 0.003394 | 0.000892 | 0.002335 | 0.002714 | 0.000298 |
| rf16 | 0.001148 | 0.000647 | 0 | 0 | 0 | 0.001888 | 0.003263 | 0.001993 | 0.007462 | 0.000551 |
| rf17 | 0.004242 | 0.005207 | 0.007909 | 0 | 0.000394 | 0.004396 | 0.002692 | 0 | 0.000349 | 0.001744 |
| rf18 | 0 | 0.000365 | 0.005033 | 0.005812 | 0.005267 | 0.001339 | 0.003366 | 0.003825 | 0.003566 | 0.001282 |
| rf19 | 0.001463 | 0.001157 | 0.000834 | 0.002887 | 0.001473 | 0.003016 | 0.001762 | 0.001456 | 0.00262 | 0.002579 |
| rf20 | 0.00035 | 0 | 0.000378 | 0.002398 | 0.001289 | 0.005888 | 0.004065 | 0.004011 | 0.004141 | 0.006701 |
| rf21 | 0.003495 | 0 | 0.000559 | 0.001171 | 0 | 0.006693 | 0.003648 | 0.002405 | 0.002092 | 0 |
| rf22 | 0 | 0.002321 | 0.001299 | 0.002767 | 0.001892 | 0.003027 | 7.77E−05 | 0.00584 | 0.001206 | 0.002339 |
| rf23 | 0.003048 | 0.007102 | 0 | 0 | 0.002513 | 0.004599 | 0.00151 | 0.006174 | 0.002441 | 0 |
| rf24 | 0.00069 | 0.01181 | 0 | 0 | 0.000288 | 0.00652 | 0.00221 | 0.001317 | 0.009443 | 0.004121 |
| rf25 | 0.00211 | 0 | 0.001841 | 0.000225 | 0.00746 | 0.003257 | 0.004587 | 0.000374 | 0.002453 | 3.18E−19 |
| rf26 | 0.002828 | 0.001188 | 0.002249 | 0 | 0 | 4.14E−05 | 0.004817 | 0.008764 | 0.009296 | 0.000763 |
| rf27 | 0.00443 | 0.007098 | 0.000925 | 0 | 0.006391 | 0.004752 | 0.003646 | 0.001479 | 0.001448 | 0 |
| extraTrees0 | 0.001094 | 0.001663 | 0.004028 | 0.000466 | 0.000388 | 0.005189 | 0.005774 | 0.002947 | 0.000727 | 0.003004 |
| extraTrees1 | 0.001942 | 0.007284 | 0.001595 | 0.003996 | 0.001509 | 0.002711 | 0.003419 | 0.007849 | 0.004629 | 0.002623 |
| extraTrees2 | 0.001887 | 0.002364 | 0.001914 | 0.002809 | 0.002438 | 0.002767 | 0.004665 | 0.00357 | 0.001712 | 0.001361 |
| extraTrees3 | 0.001955 | 0.004606 | 0.003287 | 0.001921 | 0.000326 | 0.002111 | 0.001725 | 0.006088 | 6.09E−05 | 0.001658 |
| extraTrees4 | 0.002347 | 0.001527 | 0.007311 | 0.000981 | 0.000892 | 0.004418 | 0.001819 | 0.002468 | 0.001365 | 0.001838 |
| extraTrees5 | 0.002529 | 0.005211 | 0.002861 | 0.002023 | 0.001502 | 0.002165 | 0.005647 | 0.00791 | 0.000956 | 0.003315 |
| extraTrees6 | 0.003517 | 0.004146 | 0.005495 | 0.002493 | 0.002976 | 0.002792 | 0.004036 | 0.007172 | 0.004238 | 0.000993 |
| extraTrees7 | 0.000599 | 0.002686 | 0.001354 | 0.001407 | 0.001602 | 0.002829 | 0.001413 | 0.005931 | 0.002988 | 0.003135 |
| extraTrees8 | 0.00103 | 0.006331 | 0.005299 | 0.00111 | 0 | 0.003682 | 0.006901 | 0.004057 | 0.002776 | 0.001522 |
| extraTrees9 | 0.002677 | 0.004004 | 0.000782 | 0.000495 | 0.000219 | 0.004121 | 0.001244 | 0.003154 | 0.003667 | 0.001995 |
| extraTrees10 | 0.002127 | 0.003109 | 0.004013 | 0.0021 | 0.001923 | 0.002194 | 0.003068 | 0.008129 | 0.00717 | 0.003518 |
| extraTrees11 | 0.000767 | 0.005395 | 0.004215 | 0.001476 | 0.001115 | 0.004834 | 0.005496 | 0.003123 | 0.003982 | 0.001259 |
| extraTrees12 | 0.001683 | 0.002036 | 0.003837 | 0.002593 | 0.003043 | 0.005328 | 0.006579 | 0.013362 | 0.008456 | 0.001511 |
| extraTrees13 | 0.002751 | 0.002658 | 0.006314 | 0.001738 | 0.002064 | 0.004488 | 0.005873 | 0.005817 | 0.0019 | 0.007553 |
| extraTrees14 | 0.001094 | 0.001663 | 0.004028 | 0.000466 | 0.000388 | 0.005189 | 0.005774 | 0.002947 | 0.000727 | 0.003004 |
| extraTrees15 | 0.001942 | 0.007284 | 0.001595 | 0.003996 | 0.001509 | 0.002711 | 0.003419 | 0.007849 | 0.004629 | 0.002623 |
| extraTrees16 | 0.001887 | 0.002364 | 0.001914 | 0.002809 | 0.002438 | 0.002767 | 0.004665 | 0.00357 | 0.001712 | 0.001361 |
| extraTrees17 | 0.001955 | 0.004606 | 0.003287 | 0.001921 | 0.000326 | 0.002111 | 0.001725 | 0.006088 | 6.09E−05 | 0.001658 |
| extraTrees18 | 0.002347 | 0.001527 | 0.007311 | 0.000981 | 0.000892 | 0.004418 | 0.001819 | 0.002468 | 0.001365 | 0.001838 |
| extraTrees19 | 0.002529 | 0.005211 | 0.002861 | 0.002023 | 0.001502 | 0.002165 | 0.005647 | 0.00791 | 0.000956 | 0.003315 |
| extraTrees20 | 0.003517 | 0.004146 | 0.005495 | 0.002493 | 0.002976 | 0.002792 | 0.004036 | 0.007172 | 0.004238 | 0.000993 |
| extraTrees21 | 0.000599 | 0.002686 | 0.001354 | 0.001407 | 0.001602 | 0.002829 | 0.001413 | 0.005931 | 0.002988 | 0.003135 |
| extraTrees22 | 0.00103 | 0.006331 | 0.005299 | 0.00111 | 0 | 0.003682 | 0.006901 | 0.004057 | 0.002776 | 0.001522 |
| extraTrees23 | 0.002677 | 0.004004 | 0.000782 | 0.000495 | 0.000219 | 0.004121 | 0.001244 | 0.003154 | 0.003667 | 0.001995 |
| extraTrees24 | 0.002127 | 0.003109 | 0.004013 | 0.0021 | 0.001923 | 0.002194 | 0.003068 | 0.008129 | 0.00717 | 0.003518 |
| extraTrees25 | 0.000767 | 0.005395 | 0.004215 | 0.001476 | 0.001115 | 0.004834 | 0.005496 | 0.003123 | 0.003982 | 0.001259 |
| extraTrees26 | 0.001683 | 0.002036 | 0.003837 | 0.002593 | 0.003043 | 0.005328 | 0.006579 | 0.013362 | 0.008456 | 0.001511 |
| extraTrees27 | 0.002751 | 0.002658 | 0.006314 | 0.001738 | 0.002064 | 0.004488 | 0.005873 | 0.005817 | 0.0019 | 0.007553 |
| gbc0 | 0 | 3.37E−05 | 0 | 4.70E−05 | 0 | 8.74E−06 | 3.41E−05 | 0 | 8.65E−05 | 0.016943 |
| gbc1 | 5.91E−05 | 0 | 0.000432 | 5.50E−06 | 0 | 0 | 0 | 0 | 0.000462 | 4.93E−06 |
| gbc2 | 0.000425 | 0 | 3.33E−05 | 0 | 0 | 0.003744 | 0 | 0.005138 | 0 | 0 |
| gbc3 | 1.17E−05 | 0.006593 | 0.008817 | 1.40E−06 | 0 | 0.001653 | 0.004775 | 0.000633 | 0 | 0 |
| gbc4 | 0 | 1.28E−05 | 0.001077 | 3.80E−05 | 0 | 0.00079 | 0 | 0 | 0.002041 | 0.000135 |
| gbc5 | 0 | 0 | 0.001837 | 0.001335 | 0 | 2.72E−05 | 0 | 0.014612 | 0 | 0.000348 |
| gbc6 | 0.000418 | 3.57E−05 | 0.007448 | 0.000183 | 0.000479 | 0 | 0.005361 | 0 | 0 | 0 |
| gbc7 | 0 | 0 | 0.000839 | 0.001045 | 3.00E−05 | 0 | 0.002622 | 0 | 0 | 0.005297 |
| gbc8 | 6.62E−05 | 0.00224 | 0.000998 | 0 | 9.30E−05 | 7.63E−05 | 0.00038 | 0 | 7.08E−06 | 0 |
| gbc9 | 0 | 0 | 0.001414 | 0 | 0 | 0.000729 | 0 | 0.000214 | 0 | 0 |
| gbc10 | 6.10E−06 | 0 | 0.007454 | 0 | 0.001832 | 0.013457 | 0.005768 | 0.011153 | 7.45E−05 | 0 |
| gbc11 | 0.000146 | 0.000214 | 4.09E−05 | 0.005377 | 6.77E−05 | 6.92E−05 | 0.00011 | 0 | 0.021049 | 4.97E−05 |
| gbc12 | 0 | 0 | 0.006386 | 0.003659 | 0 | 0 | 0.004073 | 6.75E−05 | 0 | 0.000508 |
| gbc13 | 0 | 1.21E−05 | 0.003859 | 0.000309 | 0.000214 | 0 | 0.001574 | 0.000584 | 0.006386 | 0.000272 |
| gbc14 | 0 | 3.37E−05 | 0 | 4.70E−05 | 0 | 8.74E−06 | 3.41E−05 | 0 | 8.65E−05 | 0.016943 |
| gbc15 | 5.91E−05 | 0 | 0.000432 | 5.50E−06 | 0 | 0 | 0 | 0 | 0.000462 | 4.93E−06 |
| gbc16 | 0.000425 | 0 | 3.33E−05 | 0 | 0 | 0.003744 | 0 | 0.005138 | 0 | 0 |
| gbc17 | 1.17E−05 | 0.006593 | 0.008817 | 1.40E−06 | 0 | 0.001653 | 0.004775 | 0.000633 | 0 | 0 |
| gbc18 | 0 | 1.28E−05 | 0.001077 | 3.80E−05 | 0 | 0.00079 | 0 | 0 | 0.002041 | 0.000135 |
| gbc19 | 0 | 0 | 0.001837 | 0.001335 | 0 | 2.72E−05 | 0 | 0.014612 | 0 | 0.000348 |
| gbc20 | 0.000418 | 3.57E−05 | 0.007448 | 0.000183 | 0.000479 | 0 | 0.005361 | 0 | 0 | 0 |
| gbc21 | 0 | 0 | 0.000839 | 0.001045 | 3.00E−05 | 0 | 0.002622 | 0 | 0 | 0.005297 |
| gbc22 | 6.62E−05 | 0.00224 | 0.000998 | 0 | 9.30E−05 | 7.63E−05 | 0.00038 | 0 | 7.08E−06 | 0 |
| gbc23 | 0 | 0 | 0.001414 | 0 | 0 | 0.000729 | 0 | 0.000214 | 0 | 0 |
| gbc24 | 6.10E−06 | 0 | 0.007454 | 0 | 0.001832 | 0.013457 | 0.005768 | 0.011153 | 7.45E−05 | 0 |
| gbc25 | 0.000146 | 0.000214 | 4.09E−05 | 0.005377 | 6.77E−05 | 6.92E−05 | 0.00011 | 0 | 0.021049 | 4.97E−05 |
| gbc26 | 0 | 0 | 0.006386 | 0.003659 | 0 | 0 | 0.004073 | 6.75E−05 | 0 | 0.000508 |
| gbc27 | 0 | 1.21E−05 | 0.003859 | 0.000309 | 0.000214 | 0 | 0.001574 | 0.000584 | 0.006386 | 0.000272 |
| ada0 | 0.02 | 0 | 0.02 | 0 | 0 | 0 | 0 | 0.02 | 0 | 0 |
| ada1 | 0.02 | 0 | 0.02 | 0 | 0 | 0 | 0 | 0.02 | 0 | 0 |
| ada2 | 0.02 | 0 | 0.02 | 0 | 0 | 0 | 0 | 0.02 | 0 | 0 |
| ada3 | 0.02 | 0 | 0.02 | 0 | 0 | 0 | 0 | 0.02 | 0 | 0 |
| ada4 | 0.02 | 0 | 0.02 | 0 | 0 | 0 | 0 | 0.02 | 0 | 0 |
| ada5 | 0.02 | 0 | 0.02 | 0 | 0 | 0 | 0 | 0.02 | 0 | 0 |
| ada6 | 0.02 | 0 | 0.02 | 0 | 0 | 0 | 0 | 0.02 | 0 | 0 |
| ada7 | 0.02 | 0 | 0.02 | 0 | 0 | 0 | 0 | 0.02 | 0 | 0 |
| ada8 | 0.02 | 0 | 0.02 | 0 | 0 | 0 | 0 | 0.02 | 0 | 0 |
| ada9 | 0.02 | 0 | 0.02 | 0 | 0 | 0 | 0 | 0.02 | 0 | 0 |
| ada10 | 0.02 | 0 | 0.02 | 0 | 0 | 0 | 0 | 0.02 | 0 | 0 |
| ada11 | 0.02 | 0 | 0.02 | 0 | 0 | 0 | 0 | 0.02 | 0 | 0 |
| ada12 | 0.02 | 0 | 0.02 | 0 | 0 | 0 | 0 | 0.02 | 0 | 0 |
| ada13 | 0.02 | 0 | 0.02 | 0 | 0 | 0 | 0 | 0.02 | 0 | 0 |
| ada14 | 0.02 | 0 | 0.02 | 0 | 0 | 0 | 0 | 0.02 | 0 | 0 |
| ada15 | 0.02 | 0 | 0.02 | 0 | 0 | 0 | 0 | 0.02 | 0 | 0 |
| ada16 | 0.02 | 0 | 0.02 | 0 | 0 | 0 | 0 | 0.02 | 0 | 0 |
| ada17 | 0.02 | 0 | 0.02 | 0 | 0 | 0 | 0 | 0.02 | 0 | 0 |
| ada18 | 0.02 | 0 | 0.02 | 0 | 0 | 0 | 0 | 0.02 | 0 | 0 |
| ada19 | 0.02 | 0 | 0.02 | 0 | 0 | 0 | 0 | 0.02 | 0 | 0 |
| ada20 | 0.02 | 0 | 0.02 | 0 | 0 | 0 | 0 | 0.02 | 0 | 0 |
| ada21 | 0.02 | 0 | 0.02 | 0 | 0 | 0 | 0 | 0.02 | 0 | 0 |
| ada22 | 0.02 | 0 | 0.02 | 0 | 0 | 0 | 0 | 0.02 | 0 | 0 |
| ada23 | 0.02 | 0 | 0.02 | 0 | 0 | 0 | 0 | 0.02 | 0 | 0 |
| ada24 | 0.02 | 0 | 0.02 | 0 | 0 | 0 | 0 | 0.02 | 0 | 0 |
| ada25 | 0.02 | 0 | 0.02 | 0 | 0 | 0 | 0 | 0.02 | 0 | 0 |
| ada26 | 0.02 | 0 | 0.02 | 0 | 0 | 0 | 0 | 0.02 | 0 | 0 |
| ada27 | 0.02 | 0 | 0.02 | 0 | 0 | 0 | 0 | 0.02 | 0 | 0. |

| | | | | ZHX1- | ZNF460- | | | | | |
|---|---|---|---|---|---|---|---|---|---|---|
| Gene | VNN1 | ZBTB11 | ZFR | C8orf76 | AS1 | ZNF804A | AASDHPPT | ABHD17A | ABHD17B | ABHD18 |
| rf0 | 0.012869 | 0.010834 | 0.002423 | 0.000374 | 0 | 0.008773 | 0.000712 | 0 | 0.000685 | 0 |
| rf1 | 0.000798 | 0.010215 | 0.004622 | 0.004134 | 0 | 0 | 0.009283 | 0.000967 | 0.005969 | 0 |
| rf2 | 0 | 0.011071 | 0.000924 | 0.0046 | 0.000384 | 0 | 1.65E−18 | 0 | 0 | 0 |
| rf3 | 0.005364 | 0 | 0 | 0.003444 | 0.001082 | 0 | 0.000741 | 0 | 0.000709 | 0.003108 |
| rf4 | 0 | 0 | 0 | 0.007613 | 0.00168 | 0.001678 | 0 | 0.001058 | 0.005057 | 0 |
| rf5 | 0 | 0.004251 | 0.001466 | 0.001362 | 0.003183 | 0.001411 | 5.80E−18 | 0.006118 | 0.000812 | 0 |
| rf6 | 0 | 0.003871 | 0.00329 | 0.001053 | 0.005535 | 0.008727 | 0.008951 | 0.000646 | 0.000997 | 0 |
| rf7 | 0.004447 | 0.025066 | 0.001288 | 0.001032 | 0 | 0.004938 | 0.003532 | 0.000703 | 0 | 0 |
| rf8 | 0.007243 | 0.008164 | 0.001232 | 0.000429 | 0.001722 | 0.001591 | 0.000782 | 0 | 4.35E−18 | 0.001314 |
| rf9 | 0.001851 | 0.009431 | 0.004654 | 0 | 0 | 0.006269 | 0.005132 | 0.000608 | 0.002576 | 0.000671 |
| rf10 | 0 | 0.004629 | 0.006252 | 0 | 0 | 0.004105 | 2.46E−18 | 0.00132 | 0 | 6.14E−18 |
| rf11 | 0.000362 | 0.012578 | 0.002507 | 0 | 2.21E−18 | 0.000742 | 0.004079 | 0 | 0.000844 | 0 |
| rf12 | 0.003587 | 0.007043 | 0.000387 | 0.004659 | 0.00035 | 0.006069 | 0 | 0.001702 | 0.004289 | 0 |
| rf13 | 0.001562 | 0.005974 | 0.002892 | 0.002774 | 0 | 0.000706 | 0.000367 | 0 | 0.003956 | 6.82E−19 |
| rf14 | 0.012869 | 0.010834 | 0.002423 | 0.000374 | 0 | 0.008773 | 0.000712 | 0 | 0.000685 | 0 |
| rf15 | 0.000798 | 0.010215 | 0.004622 | 0.004134 | 0 | 0 | 0.009283 | 0.000967 | 0.005969 | 0 |
| rf16 | 0 | 0.011071 | 0.000924 | 0.0046 | 0.000384 | 0 | 1.65E−18 | 0 | 0 | 0 |
| rf17 | 0.005364 | 0 | 0 | 0.003444 | 0.001082 | 0 | 0.000741 | 0 | 0.000709 | 0.003108 |
| rf18 | 0 | 0 | 0 | 0.007613 | 0.00168 | 0.001678 | 0 | 0.001058 | 0.005057 | 0 |
| rf19 | 0 | 0.004251 | 0.001466 | 0.001362 | 0.003183 | 0.001411 | 5.80E−18 | 0.006118 | 0.000812 | 0 |
| rf20 | 0 | 0.003871 | 0.00329 | 0.001053 | 0.005535 | 0.008727 | 0.008951 | 0.000646 | 0.000997 | 0 |
| rf21 | 0.004447 | 0.025066 | 0.001288 | 0.001032 | 0 | 0.004938 | 0.003532 | 0.000703 | 0 | 0 |
| rf22 | 0.007243 | 0.008164 | 0.001232 | 0.000429 | 0.001722 | 0.001591 | 0.000782 | 0 | 4.35E−18 | 0.001314 |
| rf23 | 0.001851 | 0.009431 | 0.004654 | 0 | 0 | 0.006269 | 0.005132 | 0.000608 | 0.002576 | 0.000671 |
| rf24 | 0 | 0.004629 | 0.006252 | 0 | 0 | 0.004105 | 2.46E−18 | 0.00132 | 0 | 6.14E−18 |
| rf25 | 0.000362 | 0.012578 | 0.002507 | 0 | 2.21E−18 | 0.000742 | 0.004079 | 0 | 0.000844 | 0 |
| rf26 | 0.003587 | 0.007043 | 0.000387 | 0.004659 | 0.00035 | 0.006069 | 0 | 0.001702 | 0.004289 | 0 |
| rf27 | 0.001562 | 0.005974 | 0.002892 | 0.002774 | 0 | 0.000706 | 0.000367 | 0 | 0.003956 | 6.82E−19 |
| extraTrees0 | 0.003291 | 0.006736 | 0.001445 | 0.00028 | 0.001844 | 0.001704 | 0.001247 | 0.00137 | 0.001695 | 0.000922 |
| extraTrees1 | 0.004497 | 0.009243 | 0.004512 | 0.002122 | 0.001258 | 0.005803 | 0.004315 | 0.000971 | 0.002044 | 0.001437 |
| extraTrees2 | 0.004477 | 0.007862 | 0.004182 | 0.001892 | 0.00092 | 0.003441 | 0.002071 | 0.002388 | 0.000294 | 0.000797 |
| extraTrees3 | 0.001115 | 0.009081 | 0.000639 | 0.000868 | 3.29E−05 | 0.010183 | 0.003742 | 0.000908 | 0.000385 | 0.00174 |
| extraTrees4 | 0.000953 | 0.01439 | 0.001057 | 0.001529 | 0.000936 | 0.006639 | 0.001273 | 0.002333 | 0.001103 | 0.001549 |
| extraTrees5 | 0.001716 | 0.004693 | 0.003296 | 0.00161 | 0.001172 | 0.009578 | 0.001799 | 0.001066 | 0.002122 | 0.001365 |
| extraTrees6 | 0.006036 | 0.004933 | 0.002508 | 0.00369 | 0.000888 | 0.004128 | 0.002703 | 0.001357 | 0.001583 | 0.001822 |
| extraTrees7 | 0.000875 | 0.009495 | 0.002009 | 0.002388 | 0.002804 | 0.008087 | 0.000238 | 0.001687 | 0.001516 | 0.002942 |
| extraTrees8 | 0.001156 | 0.004616 | 0.002289 | 0.003174 | 0.00251 | 0.011986 | 0.0025 | 0.001452 | 0.001336 | 0.00106 |
| extraTrees9 | 0.001515 | 0.00565 | 0.003197 | 0.003085 | 0.002015 | 0.005957 | 0.001337 | 0.001451 | 0.000543 | 0.001452 |
| extraTrees10 | 0.003095 | 0.007212 | 0.002556 | 0.001447 | 0.000739 | 0.001576 | 0.001456 | 0.000299 | 0.001179 | 0.001563 |
| extraTrees11 | 0.001389 | 0.012526 | 0.002071 | 0.002695 | 0 | 0.003917 | 0.004215 | 0.000222 | 0.001996 | 0.001813 |
| extraTrees12 | 0.00659 | 0.006698 | 0.004648 | 0.001674 | 0.000786 | 0.005123 | 0.002432 | 0.000362 | 0.000596 | 0.001567 |
| extraTrees13 | 0.001524 | 0.009127 | 0.003227 | 0.001679 | 0.001636 | 0.006585 | 0.00219 | 0.000951 | 0.003039 | 0.000697 |
| extraTrees14 | 0.003291 | 0.006736 | 0.001445 | 0.00028 | 0.001844 | 0.001704 | 0.001247 | 0.00137 | 0.001695 | 0.000922 |
| extraTrees15 | 0.004497 | 0.009243 | 0.004512 | 0.002122 | 0.001258 | 0.005803 | 0.004315 | 0.000971 | 0.002044 | 0.001437 |
| extraTrees16 | 0.004477 | 0.007862 | 0.004182 | 0.001892 | 0.00092 | 0.003441 | 0.002071 | 0.002388 | 0.000294 | 0.000797 |
| extraTrees17 | 0.001115 | 0.009081 | 0.000639 | 0.000868 | 3.29E−05 | 0.010183 | 0.003742 | 0.000908 | 0.000385 | 0.00174 |
| extraTrees18 | 0.000953 | 0.01439 | 0.001057 | 0.001529 | 0.000936 | 0.006639 | 0.001273 | 0.002333 | 0.001103 | 0.001549 |
| extraTrees19 | 0.001716 | 0.004693 | 0.003296 | 0.00161 | 0.001172 | 0.009578 | 0.001799 | 0.001066 | 0.002122 | 0.001365 |
| extraTrees20 | 0.006036 | 0.004933 | 0.002508 | 0.00369 | 0.000888 | 0.004128 | 0.002703 | 0.001357 | 0.001583 | 0.001822 |
| extraTrees21 | 0.000875 | 0.009495 | 0.002009 | 0.002388 | 0.002804 | 0.008087 | 0.000238 | 0.001687 | 0.001516 | 0.002942 |
| extraTrees22 | 0.001156 | 0.004616 | 0.002289 | 0.003174 | 0.00251 | 0.011986 | 0.0025 | 0.001452 | 0.001336 | 0.00106 |
| extraTrees23 | 0.001515 | 0.00565 | 0.003197 | 0.003085 | 0.002015 | 0.005957 | 0.001337 | 0.001451 | 0.000543 | 0.001452 |
| extraTrees24 | 0.003095 | 0.007212 | 0.002556 | 0.001447 | 0.000739 | 0.001576 | 0.001456 | 0.000299 | 0.001179 | 0.001563 |
| extraTrees25 | 0.001389 | 0.012526 | 0.002071 | 0.002695 | 0 | 0.003917 | 0.004215 | 0.000222 | 0.001996 | 0.001813 |
| extraTrees26 | 0.00659 | 0.006698 | 0.004648 | 0.001674 | 0.000786 | 0.005123 | 0.002432 | 0.000362 | 0.000596 | 0.001567 |
| extraTrees27 | 0.001524 | 0.009127 | 0.003227 | 0.001679 | 0.001636 | 0.006585 | 0.00219 | 0.000951 | 0.003039 | 0.000697 |
| gbc0 | 0 | 0.000716 | 0.014521 | 0.000249 | 5.31E−05 | 0.004765 | 1.26E−06 | 0 | 0 | 1.56E−06 |
| gbc1 | 0.00624 | 0.003749 | 0.001643 | 0 | 0.001793 | 0.011605 | 0.025426 | 0 | 0 | 0 |
| gbc2 | 0.000589 | 0.000358 | 0.007164 | 0.000321 | 0.001261 | 0 | 0.000302 | 7.20E−06 | 0 | 0 |
| gbc3 | 0.001232 | 0.015053 | 0.000247 | 0 | 0 | 0 | 0.000564 | 0.000226 | 0.004779 | 0 |
| gbc4 | 9.61E−05 | 0.046595 | 0.000142 | 8.78E−05 | 0.008813 | 0 | 0.000407 | 7.24E−05 | 0 | 0 |
| gbc5 | 0.001704 | 0.05239 | 0.000586 | 0 | 0.020295 | 9.87E−05 | 0 | 8.12E−07 | 0.000141 | 0 |
| gbc6 | 0 | 0.009309 | 0.00238 | 0.001684 | 0 | 0 | 0.000274 | 0 | 0.00086 | 0 |
| gbc7 | 0 | 0.000397 | 0.003072 | 0 | 0 | 0 | 0.009222 | 0.005251 | 0.000181 | 0 |
| gbc8 | 3.15E−05 | 0.004996 | 0.03241 | 0.000182 | 0.00229 | 0.000124 | 0 | 0 | 0 | 0 |
| gbc9 | 0.003099 | 0.003602 | 0.0171 | 1.43E−05 | 0 | 0 | 0 | 0 | 0.000585 | 5.15E−07 |
| gbc10 | 0.007181 | 0.009131 | 0 | 0 | 0.000461 | 0.000179 | 0.004132 | 0 | 0 | 0.001491 |
| gbc11 | 0.005875 | 0 | 0 | 0.000264 | 0 | 9.45E−05 | 0.000663 | 0.000563 | 0 | 7.67E−08 |
| gbc12 | 0 | 1.86E−06 | 0.035122 | 0 | 0.006372 | 0 | 0.019272 | 0 | 0 | 0.003301 |
| gbc13 | 0.004478 | 0.031195 | 0.001488 | 9.44E−05 | 0 | 6.86E−05 | 5.82E−05 | 0.000377 | 0 | 0 |
| gbc14 | 0 | 0.000716 | 0.014521 | 0.000249 | 5.31E−05 | 0.004765 | 1.26E−06 | 0 | 0 | 1.56E−06 |
| gbc15 | 0.00624 | 0.003749 | 0.001643 | 0 | 0.001793 | 0.011605 | 0.025426 | 0 | 0 | 0 |
| gbc16 | 0.000589 | 0.000358 | 0.007164 | 0.000321 | 0.001261 | 0 | 0.000302 | 7.20E−06 | 0 | 0 |
| gbc17 | 0.001232 | 0.015053 | 0.000247 | 0 | 0 | 0 | 0.000564 | 0.000226 | 0.004779 | 0 |
| gbc18 | 9.61E−05 | 0.046595 | 0.000142 | 8.78E−05 | 0.008813 | 0 | 0.000407 | 7.24E−05 | 0 | 0 |
| gbc19 | 0.001704 | 0.05239 | 0.000586 | 0 | 0.020295 | 9.87E−05 | 0 | 8.12E−07 | 0.000141 | 0 |
| gbc20 | 0 | 0.009309 | 0.00238 | 0.001684 | 0 | 0 | 0.000274 | 0 | 0.00086 | 0 |
| gbc21 | 0 | 0.000397 | 0.003072 | 0 | 0 | 0 | 0.009222 | 0.005251 | 0.000181 | 0 |
| gbc22 | 3.15E−05 | 0.004996 | 0.03241 | 0.000182 | 0.00229 | 0.000124 | 0 | 0 | 0 | 0 |
| gbc23 | 0.003099 | 0.003602 | 0.0171 | 1.43E−05 | 0 | 0 | 0 | 0 | 0.000585 | 5.15E−07 |
| gbc24 | 0.007181 | 0.009131 | 0 | 0 | 0.000461 | 0.000179 | 0.004132 | 0 | 0 | 0.001491 |
| gbc25 | 0.005875 | 0 | 0 | 0.000264 | 0 | 9.45E−05 | 0.000663 | 0.000563 | 0 | 7.67E−08 |
| gbc26 | 0 | 1.86E−06 | 0.035122 | 0 | 0.006372 | 0 | 0.019272 | 0 | 0 | 0.003301 |
| gbc27 | 0.004478 | 0.031195 | 0.001488 | 9.44E−05 | 0 | 6.86E−05 | 5.82E−05 | 0.000377 | 0 | 0 |
| ada0 | 0 | 0.04 | 0 | 0 | 0 | 0.02 | 0 | 0 | 0 | 0 |
| ada1 | 0 | 0.04 | 0 | 0 | 0 | 0.02 | 0 | 0 | 0 | 0 |
| ada2 | 0 | 0.04 | 0 | 0 | 0 | 0.02 | 0 | 0 | 0 | 0 |
| ada3 | 0 | 0.04 | 0 | 0 | 0 | 0.02 | 0 | 0 | 0 | 0 |
| ada4 | 0 | 0.04 | 0 | 0 | 0 | 0.02 | 0 | 0 | 0 | 0 |
| ada5 | 0 | 0.04 | 0 | 0 | 0 | 0.02 | 0 | 0 | 0 | 0 |
| ada6 | 0 | 0.04 | 0 | 0 | 0 | 0.02 | 0 | 0 | 0 | 0 |
| ada7 | 0 | 0.04 | 0 | 0 | 0 | 0.02 | 0 | 0 | 0 | 0 |
| ada8 | 0 | 0.04 | 0 | 0 | 0 | 0.02 | 0 | 0 | 0 | 0 |
| ada9 | 0 | 0.04 | 0 | 0 | 0 | 0.02 | 0 | 0 | 0 | 0 |
| ada10 | 0 | 0.04 | 0 | 0 | 0 | 0.02 | 0 | 0 | 0 | 0 |
| ada11 | 0 | 0.04 | 0 | 0 | 0 | 0.02 | 0 | 0 | 0 | 0 |
| ada12 | 0 | 0.04 | 0 | 0 | 0 | 0.02 | 0 | 0 | 0 | 0 |
| ada13 | 0 | 0.04 | 0 | 0 | 0 | 0.02 | 0 | 0 | 0 | 0 |
| ada14 | 0 | 0.04 | 0 | 0 | 0 | 0.02 | 0 | 0 | 0 | 0 |
| ada15 | 0 | 0.04 | 0 | 0 | 0 | 0.02 | 0 | 0 | 0 | 0 |
| ada16 | 0 | 0.04 | 0 | 0 | 0 | 0.02 | 0 | 0 | 0 | 0 |
| ada17 | 0 | 0.04 | 0 | 0 | 0 | 0.02 | 0 | 0 | 0 | 0 |
| ada18 | 0 | 0.04 | 0 | 0 | 0 | 0.02 | 0 | 0 | 0 | 0 |
| ada19 | 0 | 0.04 | 0 | 0 | 0 | 0.02 | 0 | 0 | 0 | 0 |
| ada20 | 0 | 0.04 | 0 | 0 | 0 | 0.02 | 0 | 0 | 0 | 0 |
| ada21 | 0 | 0.04 | 0 | 0 | 0 | 0.02 | 0 | 0 | 0 | 0 |
| ada22 | 0 | 0.04 | 0 | 0 | 0 | 0.02 | 0 | 0 | 0 | 0 |
| ada23 | 0 | 0.04 | 0 | 0 | 0 | 0.02 | 0 | 0 | 0 | 0 |
| ada24 | 0 | 0.04 | 0 | 0 | 0 | 0.02 | 0 | 0 | 0 | 0 |
| ada25 | 0 | 0.04 | 0 | 0 | 0 | 0.02 | 0 | 0 | 0 | 0 |
| ada26 | 0 | 0.04 | 0 | 0 | 0 | 0.02 | 0 | 0 | 0 | 0 |
| ada27 | 0 | 0.04 | 0 | 0 | 0 | 0.02 | 0 | 0 | 0 | 0 |

| Gene | AC004053.1 | AC006213.2 | AC016642.1 | AC062015.1 | AC092718.4 | AC093673.1 | AC096577.1 | AC104248.1 | AC106865.1 | AC118549.1 |
|---|---|---|---|---|---|---|---|---|---|---|
| rf0 | 0.002807 | 3.16E−18 | 0 | 0.004544 | 0.0006 | 0.001129 | 0.004427 | 0 | 0 | 0 |
| rf1 | 0.008163 | 4.59E−18 | 0 | 0 | 2.61E−19 | 0 | 0.003145 | 0.002637 | 0.001039 | 0 |
| rf2 | 0.000629 | 0.00132 | 0.005979 | 0.001279 | 0.008409 | 0 | 0.001894 | 0.000904 | 0.002133 | 0.001797 |
| rf3 | 0 | 4.64E−05 | 0 | 0 | 1.04E−18 | 0.000507 | 0.001798 | 0.005097 | 0.002724 | 0 |
| rf4 | 2.55E−18 | 0.009389 | 0 | 0.005717 | 0 | 0.002124 | 0.004506 | 3.10E−19 | 0.001877 | 5.17E−18 |
| rf5 | 0.003681 | 0.001081 | 0.003205 | 0.004503 | 0.006689 | 0.001414 | 0.005303 | 0 | 0 | 0 |
| rf6 | 0.004294 | 0.001455 | 0.00178 | 0 | 0.001291 | 0.001819 | 0 | 0 | 0.001168 | 0 |
| rf7 | 0.000285 | 0.000331 | 0.002355 | 0.002903 | 0 | 0.006222 | 0.004206 | 0 | 0.003265 | 0.0018 |
| rf8 | 0 | 0.000607 | 0.000737 | 0.00094 | 0 | 0.001783 | 0.001702 | 0 | 0.004109 | 0 |
| rf9 | 0.001481 | 0.00508 | 0.000768 | 0.001458 | 0.003095 | 0.007546 | 0.000485 | 0 | 0.002303 | 0.001582 |
| rf10 | 0 | 0.006186 | 0.001674 | 0.002362 | 3.61E−18 | 5.69E−18 | 0 | 0.001347 | 0 | 0.000858 |
| rf11 | 0.005889 | 0.00116 | 0.005118 | 0.003314 | 0.000364 | 0.000784 | 0.007287 | 0.003736 | 0.001381 | 0.006803 |
| rf12 | 2.67E−19 | 0 | 0.001665 | 0.000339 | 0.000825 | 0.003114 | 0.003905 | 0.002904 | 0 | 0 |
| rf13 | 0.001489 | 0.000339 | 0 | 0 | 0.000595 | 0.000902 | 0.000599 | 0.002173 | 0.004291 | 0.00222 |
| rf14 | 0.002807 | 3.16E−18 | 0 | 0.004544 | 0.0006 | 0.001129 | 0.004427 | 0 | 0 | 0 |
| rf15 | 0.008163 | 4.59E−18 | 0 | 0 | 2.61E−19 | 0 | 0.003145 | 0.002637 | 0.001039 | 0 |
| rf16 | 0.000629 | 0.00132 | 0.005979 | 0.001279 | 0.008409 | 0 | 0.001894 | 0.000904 | 0.002133 | 0.001797 |
| rf17 | 0 | 4.64E−05 | 0 | 0 | 1.04E−18 | 0.000507 | 0.001798 | 0.005097 | 0.002724 | 0 |
| rf18 | 2.55E−18 | 0.009389 | 0 | 0.005717 | 0 | 0.002124 | 0.004506 | 3.10E−19 | 0.001877 | 5.17E−18 |
| rf19 | 0.003681 | 0.001081 | 0.003205 | 0.004503 | 0.006689 | 0.001414 | 0.005303 | 0 | 0 | 0 |
| rf20 | 0.004294 | 0.001455 | 0.00178 | 0 | 0.001291 | 0.001819 | 0 | 0 | 0.001168 | 0 |
| rf21 | 0.000285 | 0.000331 | 0.002355 | 0.002903 | 0 | 0.006222 | 0.004206 | 0 | 0.003265 | 0.0018 |
| rf22 | 0 | 0.000607 | 0.000737 | 0.00094 | 0 | 0.001783 | 0.001702 | 0 | 0.004109 | 0 |
| rf23 | 0.001481 | 0.00508 | 0.000768 | 0.001458 | 0.003095 | 0.007546 | 0.000485 | 0 | 0.002303 | 0.001582 |
| rf24 | 0 | 0.006186 | 0.001674 | 0.002362 | 3.61E−18 | 5.69E−18 | 0 | 0.001347 | 0 | 0.000858 |
| rf25 | 0.005889 | 0.00116 | 0.005118 | 0.003314 | 0.000364 | 0.000784 | 0.007287 | 0.003736 | 0.001381 | 0.006803 |
| rf26 | 2.67E−19 | 0 | 0.001665 | 0.000339 | 0.000825 | 0.003114 | 0.003905 | 0.002904 | 0 | 0 |
| rf27 | 0.001489 | 0.000339 | 0 | 0 | 0.000595 | 0.000902 | 0.000599 | 0.002173 | 0.004291 | 0.00222 |
| extraTrees0 | 0.001736 | 0.001866 | 0.000476 | 0.00026 | 0.001269 | 0.002516 | 0.005192 | 0.00201 | 0.001343 | 0.002464 |
| extraTrees1 | 0.004475 | 0.003117 | 0.002479 | 0.002369 | 0.00148 | 0.001991 | 0.002995 | 0.001882 | 0.002109 | 0.000513 |
| extraTrees2 | 0.003312 | 0.0014 | 0.00067 | 0.001493 | 0.002764 | 0.002022 | 0.003865 | 0.002544 | 0.000955 | 0.000431 |
| extraTrees3 | 0.003833 | 0.001963 | 0.002792 | 0.004559 | 0.000788 | 0.001168 | 0.002501 | 0.00187 | 0.002236 | 0.002672 |
| extraTrees4 | 0.001012 | 6.16E−05 | 0.003452 | 0.002371 | 0.000722 | 0.001101 | 0.007346 | 0.000817 | 0.001601 | 0.001011 |
| extraTrees5 | 0.00469 | 0.002368 | 0.001065 | 0.001637 | 0.002282 | 0.000567 | 0.003419 | 0.001391 | 0.002402 | 0.002388 |
| extraTrees6 | 1.23E−18 | 0.001065 | 0.00125 | 0.002782 | 0.000783 | 0.001366 | 0.002713 | 0.002892 | 0.002202 | 0.00178 |
| extraTrees7 | 0.001248 | 0.003999 | 0.00307 | 0.002061 | 0.000696 | 0.00113 | 0.004938 | 0.000299 | 0.001642 | 0.00047 |
| extraTrees8 | 0.000349 | 0.002875 | 0.002714 | 0.001527 | 0.000884 | 0.001949 | 0.00224 | 0.001527 | 0.005103 | 0.001527 |
| extraTrees9 | 0.00169 | 0.000769 | 0.000745 | 0.001827 | 0.001078 | 0.000763 | 0.001126 | 0.000351 | 0.002662 | 0 |
| extraTrees10 | 0.001727 | 0.004788 | 0.001257 | 0.003002 | 0.001747 | 0.00071 | 0.001615 | 0.001754 | 0.002773 | 0 |
| extraTrees11 | 0.0019 | 0.004506 | 0.002221 | 0.001254 | 0.000946 | 0.001907 | 0.000612 | 0.002985 | 0.000651 | 0.003484 |
| extraTrees12 | 0.004814 | 0.004553 | 0.002304 | 0.000591 | 0.00213 | 0.000334 | 0.002969 | 0.000337 | 0.000566 | 0.000515 |
| extraTrees13 | 0.00192 | 0.001599 | 0.00511 | 0.001424 | 0.003404 | 0.001409 | 0.002092 | 0.001068 | 0.002236 | 0.001712 |
| extraTrees14 | 0.001736 | 0.001866 | 0.000476 | 0.00026 | 0.001269 | 0.002516 | 0.005192 | 0.00201 | 0.001343 | 0.002464 |
| extraTrees15 | 0.004475 | 0.003117 | 0.002479 | 0.002369 | 0.00148 | 0.001991 | 0.002995 | 0.001882 | 0.002109 | 0.000513 |
| extraTrees16 | 0.003312 | 0.0014 | 0.00067 | 0.001493 | 0.002764 | 0.002022 | 0.003865 | 0.002544 | 0.000955 | 0.000431 |
| extraTrees17 | 0.003833 | 0.001963 | 0.002792 | 0.004559 | 0.000788 | 0.001168 | 0.002501 | 0.00187 | 0.002236 | 0.002672 |
| extraTrees18 | 0.001012 | 6.16E−05 | 0.003452 | 0.002371 | 0.000722 | 0.001101 | 0.007346 | 0.000817 | 0.001601 | 0.001011 |
| extraTrees19 | 0.00469 | 0.002368 | 0.001065 | 0.001637 | 0.002282 | 0.000567 | 0.003419 | 0.001391 | 0.002402 | 0.002388 |
| extraTrees20 | 1.23E−18 | 0.001065 | 0.00125 | 0.002782 | 0.000783 | 0.001366 | 0.002713 | 0.002892 | 0.002202 | 0.00178 |
| extraTrees21 | 0.001248 | 0.003999 | 0.00307 | 0.002061 | 0.000696 | 0.00113 | 0.004938 | 0.000299 | 0.001642 | 0.00047 |
| extraTrees22 | 0.000349 | 0.002875 | 0.002714 | 0.001527 | 0.000884 | 0.001949 | 0.00224 | 0.001527 | 0.005103 | 0.001527 |
| extraTrees23 | 0.00169 | 0.000769 | 0.000745 | 0.001827 | 0.001078 | 0.000763 | 0.001126 | 0.000351 | 0.002662 | 0 |
| extraTrees24 | 0.001727 | 0.004788 | 0.001257 | 0.003002 | 0.001747 | 0.00071 | 0.001615 | 0.001754 | 0.002773 | 0 |
| extraTrees25 | 0.0019 | 0.004506 | 0.002221 | 0.001254 | 0.000946 | 0.001907 | 0.000612 | 0.002985 | 0.000651 | 0.003484 |
| extraTrees26 | 0.004814 | 0.004553 | 0.002304 | 0.000591 | 0.00213 | 0.000334 | 0.002969 | 0.000337 | 0.000566 | 0.000515 |
| extraTrees27 | 0.00192 | 0.001599 | 0.00511 | 0.001424 | 0.003404 | 0.001409 | 0.002092 | 0.001068 | 0.002236 | 0.001712 |
| gbc0 | 0 | 1.90E−07 | 0 | 3.22E−05 | 0 | 0.002411 | 0 | 0 | 0 | 0 |
| gbc1 | 0.007638 | 5.27E−05 | 0 | 0 | 0 | 6.09E−05 | 0 | 0 | 0.019379 | 0.000606 |
| gbc2 | 0.008005 | 0 | 0 | 0 | 0 | 0.006199 | 5.09E−05 | 0 | 0 | 0 |
| gbc3 | 6.94E−05 | 0 | 0 | 0.010448 | 0 | 0 | 0 | 0 | 0.005325 | 0 |
| gbc4 | 0 | 0.007296 | 0.001034 | 4.39E−05 | 2.76E−05 | 0.000687 | 0 | 0.00535 | 0 | 0 |
| gbc5 | 0 | 0 | 0 | 0.000108 | 0 | 0 | 0.016906 | 0 | 0.014062 | 0 |
| gbc6 | 0 | 0 | 0.000164 | 0.000494 | 0 | 3.90E−05 | 0 | 0.002767 | 0 | 0 |
| gbc7 | 0.000436 | 0.002475 | 0 | 0 | 0 | 0.000137 | 0.000902 | 0 | 0 | 0 |
| gbc8 | 4.37E−05 | 0 | 0.005481 | 0 | 0 | 0.00082 | 0.00306 | 5.22E−05 | 0.001852 | 0 |
| gbc9 | 3.37E−05 | 0 | 0.005286 | 1.60E−05 | 0 | 0 | 0.000114 | 0 | 2.84E−05 | 0 |
| gbc10 | 0 | 0 | 0 | 0 | 0.001995 | 6.19E−08 | 3.08E−05 | 0 | 1.87E−05 | 0.000158 |
| gbc11 | 0 | 0 | 0 | 0 | 2.28E−05 | 1.55E−06 | 0 | 0 | 7.50E−05 | 0.000161 |
| gbc12 | 0 | 0 | 3.79E−05 | 0 | 0 | 0 | 0 | 0 | 1.66E−05 | 0 |
| gbc13 | 0 | 0.011609 | 1.01E−05 | 1.45E−06 | 0.000152 | 0 | 0.000257 | 0 | 0.007239 | 0.00054 |
| gbc14 | 0 | 1.90E−07 | 0 | 3.22E−05 | 0 | 0.002411 | 0 | 0 | 0 | 0 |
| gbc15 | 0.007638 | 5.27E−05 | 0 | 0 | 0 | 6.09E−05 | 0 | 0 | 0.019379 | 0.000606 |
| gbc16 | 0.008005 | 0 | 0 | 0 | 0 | 0.006199 | 5.09E−05 | 0 | 0 | 0 |
| gbc17 | 6.94E−05 | 0 | 0 | 0.010448 | 0 | 0 | 0 | 0 | 0.005325 | 0 |
| gbc18 | 0 | 0.007296 | 0.001034 | 4.39E−05 | 2.76E−05 | 0.000687 | 0 | 0.00535 | 0 | 0 |
| gbc19 | 0 | 0 | 0 | 0.000108 | 0 | 0 | 0.016906 | 0 | 0.014062 | 0 |
| gbc20 | 0 | 0 | 0.000164 | 0.000494 | 0 | 3.90E−05 | 0 | 0.002767 | 0 | 0 |
| gbc21 | 0.000436 | 0.002475 | 0 | 0 | 0 | 0.000137 | 0.000902 | 0 | 0 | 0 |
| gbc22 | 4.37E−05 | 0 | 0.005481 | 0 | 0 | 0.00082 | 0.00306 | 5.22E−05 | 0.001852 | 0 |
| gbc23 | 3.37E−05 | 0 | 0.005286 | 1.60E−05 | 0 | 0 | 0.000114 | 0 | 2.84E−05 | 0 |
| gbc24 | 0 | 0 | 0 | 0 | 0.001995 | 6.19E−08 | 3.08E−05 | 0 | 1.87E−05 | 0.000158 |
| gbc25 | 0 | 0 | 0 | 0 | 2.28E−05 | 1.55E−06 | 0 | 0 | 7.50E−05 | 0.000161 |
| gbc26 | 0 | 0 | 3.79E−05 | 0 | 0 | 0 | 0 | 0 | 1.66E−05 | 0 |
| gbc27 | 0 | 0.011609 | 1.01E−05 | 1.45E−06 | 0.000152 | 0 | 0.000257 | 0 | 0.007239 | 0.00054 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | AC120114.1 | ACADSB | ACAP2 | ACER2 | AFF4 | AHR | AK6 | AL049828.1 | AL133163.2 | AL136382.1 |
|---|---|---|---|---|---|---|---|---|---|---|
| rf0 | 0.000339 | 0 | 3.94E−18 | 0.008584 | 0.000347 | 0 | 1.98E−18 | 0.01027 | 0.001704 | 0.000342 |
| rf1 | 0 | 0.000287 | 0.000384 | 0.000563 | 0.001234 | 0.000638 | 0 | 0.003843 | 0 | 0.002669 |
| rf2 | 0.000279 | 0 | 0.002617 | 0.005669 | 0.000875 | 0.012831 | 0.000379 | 0.001361 | 0 | 0 |
| rf3 | 0.000745 | 0.000404 | 0 | 0.001204 | 0 | 0.001562 | 0.001368 | 0.002622 | 0 | 0.00109 |
| rf4 | 0.001072 | 0.000923 | 0.002022 | 0.00054 | 0.000599 | 0 | 0 | 0.004476 | 0 | 0 |
| rf5 | 0 | 2.74E−18 | 0.004183 | 0 | 8.76E−19 | 0 | 0 | 0 | 0.001877 | 0 |
| rf6 | 0 | 4.18E−18 | 0 | 0.005894 | 1.32E−18 | 0.011318 | 0.002383 | 0.002094 | 0.001083 | 0 |
| rf7 | 0.005175 | 0 | 5.17E−19 | 0.010516 | 0.000861 | 0.012573 | 0.000672 | 0 | 0 | 0 |
| rf8 | 0.00036 | 0.001673 | 2.47E−18 | 0.003497 | 0.001003 | 0.006115 | 0.003615 | 0.001199 | 2.22E−18 | 0 |
| rf9 | 0 | 0.000832 | 0 | 0.000348 | 0 | 0.004767 | 0.002442 | 0.001397 | 0 | 0.000298 |
| rf10 | 0.001309 | 0.000476 | 0 | 0.00515 | 0.001165 | 0.000449 | 0 | 0.005688 | 0 | 0.000732 |
| rf11 | 0.002215 | 0.000709 | 0 | 0.000726 | 0.00222 | 0.001322 | 0.000702 | 0.00453 | 2.97E−18 | 0.002585 |
| rf12 | 0.000674 | 2.52E−19 | 0 | 8.92E−19 | 0.001495 | 0 | 0.002297 | 0.000918 | 0.001825 | 0 |
| rf13 | 0.003192 | 0 | 0 | 0.002664 | 0 | 0.006659 | 0.00065 | 4.84E−18 | 0.002262 | 0.000677 |
| rf14 | 0.000339 | 0 | 3.94E−18 | 0.008584 | 0.000347 | 0 | 1.98E−18 | 0.01027 | 0.001704 | 0.000342 |
| rf15 | 0 | 0.000287 | 0.000384 | 0.000563 | 0.001234 | 0.000638 | 0 | 0.003843 | 0 | 0.002669 |
| rf16 | 0.000279 | 0 | 0.002617 | 0.005669 | 0.000875 | 0.012831 | 0.000379 | 0.001361 | 0 | 0 |
| rf17 | 0.000745 | 0.000404 | 0 | 0.001204 | 0 | 0.001562 | 0.001368 | 0.002622 | 0 | 0.00109 |
| rf18 | 0.001072 | 0.000923 | 0.002022 | 0.00054 | 0.000599 | 0 | 0 | 0.004476 | 0 | 0 |
| rf19 | 0 | 2.74E−18 | 0.004183 | 0 | 8.76E−19 | 0 | 0 | 0 | 0.001877 | 0 |
| rf20 | 0 | 4.18E−18 | 0 | 0.005894 | 1.32E−18 | 0.011318 | 0.002383 | 0.002094 | 0.001083 | 0 |
| rf21 | 0.005175 | 0 | 5.17E−19 | 0.010516 | 0.000861 | 0.012573 | 0.000672 | 0 | 0 | 0 |
| rf22 | 0.00036 | 0.001673 | 2.47E−18 | 0.003497 | 0.001003 | 0.006115 | 0.003615 | 0.001199 | 2.22E−18 | 0 |
| rf23 | 0 | 0.000832 | 0 | 0.000348 | 0 | 0.004767 | 0.002442 | 0.001397 | 0 | 0.000298 |
| rf24 | 0.001309 | 0.000476 | 0 | 0.00515 | 0.001165 | 0.000449 | 0 | 0.005688 | 0 | 0.000732 |
| rf25 | 0.002215 | 0.000709 | 0 | 0.000726 | 0.00222 | 0.001322 | 0.000702 | 0.00453 | 2.97E−18 | 0.002585 |
| rf26 | 0.000674 | 2.52E−19 | 0 | 8.92E−19 | 0.001495 | 0 | 0.002297 | 0.000918 | 0.001825 | 0 |
| rf27 | 0.003192 | 0 | 0 | 0.002664 | 0 | 0.006659 | 0.00065 | 4.84E−18 | 0.002262 | 0.000677 |
| extraTrees0 | 0.000554 | 0.000492 | 0.001994 | 0.00505 | 0.002461 | 0.002717 | 0.002534 | 0.001995 | 0.000961 | 0.000585 |
| extraTrees1 | 0 | 0.000721 | 0 | 0.00363 | 0.001408 | 0.005194 | 0.001133 | 0.012175 | 0.001921 | 0.001588 |
| extraTrees2 | 0 | 0.000714 | 0.000932 | 0.001853 | 0.001792 | 0.003614 | 0.001688 | 0.004935 | 0.001448 | 0.00169 |
| extraTrees3 | 0.001046 | 0.001026 | 0.001671 | 0.003975 | 0.003295 | 0.005158 | 0.000394 | 0.001745 | 0.00133 | 0.000625 |
| extraTrees4 | 0.000234 | 0.002326 | 0.00025 | 0.003295 | 0.007708 | 0.002455 | 0.001922 | 0.00493 | 0.00028 | 0.002869 |
| extraTrees5 | 0.000803 | 0.000473 | 0.00178 | 0.002043 | 0.004175 | 0.001773 | 0.001319 | 0.009769 | 0.000784 | 0.00169 |
| extraTrees6 | 0.001376 | 0.000756 | 0.000238 | 0.00981 | 0.001804 | 0.002721 | 0.000398 | 0.006361 | 0.002855 | 0.000293 |
| extraTrees7 | 0.001279 | 0.001574 | 5.85E−05 | 0.003364 | 0.003401 | 0.002577 | 5.58E−05 | 0.00254 | 0.000903 | 0.000869 |
| extraTrees8 | 0.001696 | 0.000898 | 0.000238 | 0.005076 | 0.001858 | 0.003034 | 0.000926 | 0.008781 | 0.000767 | 0.002117 |
| extraTrees9 | 0.003157 | 0.000326 | 0.001282 | 0.003354 | 0.000941 | 0.004372 | 0.0003 | 0.00841 | 0.002102 | 0.000732 |
| extraTrees10 | 0.000318 | 0.001359 | 0.00183 | 0.00616 | 1.08E−18 | 0.001374 | 0.003024 | 0.005798 | 0.001135 | 0.000515 |
| extraTrees11 | 0.003421 | 0.000238 | 0.001147 | 0.004179 | 0.00162 | 0.002567 | 0.000707 | 0.003335 | 0.001045 | 0.004108 |
| extraTrees12 | 0.000609 | 0.00216 | 0.002035 | 0.007652 | 0.001057 | 0.001492 | 0.001257 | 0.006452 | 0.001119 | 0.000267 |
| extraTrees13 | 1.26E−18 | 0.001153 | 0.002752 | 0.001699 | 0.001101 | 0.00059 | 0.000264 | 0.002887 | 0.000995 | 0.000257 |
| extraTrees14 | 0.000554 | 0.000492 | 0.001994 | 0.00505 | 0.002461 | 0.002717 | 0.002534 | 0.001995 | 0.000961 | 0.000585 |
| extraTrees15 | 0 | 0.000721 | 0 | 0.00363 | 0.001408 | 0.005194 | 0.001133 | 0.012175 | 0.001921 | 0.001588 |
| extraTrees16 | 0 | 0.000714 | 0.000932 | 0.001853 | 0.001792 | 0.003614 | 0.001688 | 0.004935 | 0.001448 | 0.00169 |
| extraTrees17 | 0.001046 | 0.001026 | 0.001671 | 0.003975 | 0.003295 | 0.005158 | 0.000394 | 0.001745 | 0.00133 | 0.000625 |
| extraTrees18 | 0.000234 | 0.002326 | 0.00025 | 0.003295 | 0.007708 | 0.002455 | 0.001922 | 0.00493 | 0.00028 | 0.002869 |
| extraTrees19 | 0.000803 | 0.000473 | 0.00178 | 0.002043 | 0.004175 | 0.001773 | 0.001319 | 0.009769 | 0.000784 | 0.00169 |
| extraTrees20 | 0.001376 | 0.000756 | 0.000238 | 0.00981 | 0.001804 | 0.002721 | 0.000398 | 0.006361 | 0.002855 | 0.000293 |
| extraTrees21 | 0.001279 | 0.001574 | 5.85E−05 | 0.003364 | 0.003401 | 0.002577 | 5.58E−05 | 0.00254 | 0.000903 | 0.000869 |
| extraTrees22 | 0.001696 | 0.000898 | 0.000238 | 0.005076 | 0.001858 | 0.003034 | 0.000926 | 0.008781 | 0.000767 | 0.002117 |
| extraTrees23 | 0.003157 | 0.000326 | 0.001282 | 0.003354 | 0.000941 | 0.004372 | 0.0003 | 0.00841 | 0.002102 | 0.000732 |
| extraTrees24 | 0.000318 | 0.001359 | 0.00183 | 0.00616 | 1.08E−18 | 0.001374 | 0.003024 | 0.005798 | 0.001135 | 0.000515 |
| extraTrees25 | 0.003421 | 0.000238 | 0.001147 | 0.004179 | 0.00162 | 0.002567 | 0.000707 | 0.003335 | 0.001045 | 0.004108 |
| extraTrees26 | 0.000609 | 0.00216 | 0.002035 | 0.007652 | 0.001057 | 0.001492 | 0.001257 | 0.006452 | 0.001119 | 0.000267 |
| extraTrees27 | 1.26E−18 | 0.001153 | 0.002752 | 0.001699 | 0.001101 | 0.00059 | 0.000264 | 0.002887 | 0.000995 | 0.000257 |
| gbc0 | 0 | 0.000136 | 0.000264 | 0.006717 | 0.004227 | 5.96E−05 | 5.31E−05 | 0.000425 | 0 | 0.000792 |
| gbc1 | 2.70E−05 | 0 | 0 | 0.000798 | 0 | 0.000944 | 8.81E−05 | 0.016653 | 2.42E−07 | 0.002672 |
| gbc2 | 0 | 1.32E−05 | 0.000114 | 0.001156 | 0 | 0.015451 | 5.35E−05 | 0.00021 | 0 | 0.000258 |
| gbc3 | 0 | 0.000861 | 2.34E−05 | 0 | 0 | 0.000146 | 1.02E−05 | 0.000247 | 0.003013 | 5.07E−05 |
| gbc4 | 7.20E−05 | 7.29E−05 | 0 | 0.002564 | 0 | 5.53E−05 | 0 | 0 | 3.85E−05 | 0.005421 |
| gbc5 | 2.50E−05 | 4.19E−07 | 0.000165 | 6.67E−05 | 0.014178 | 0.000697 | 0 | 0 | 0 | 0 |
| gbc6 | 0 | 0.000722 | 4.64E−05 | 2.63E−05 | 0 | 0.000697 | 0 | 0.003086 | 0 | 0.000187 |
| gbc7 | 0 | 0 | 0 | 0.01342 | 2.13E−05 | 0.009249 | 0 | 0.00064 | 0.001935 | 0 |
| gbc8 | 0 | 0 | 0.001417 | 0.000167 | 0.003341 | 0 | 0 | 0.000947 | 0.001989 | 0.000105 |
| gbc9 | 0 | 0 | 0.000172 | 0.000164 | 0 | 1.36E−05 | 0 | 0.000359 | 0.000956 | 0 |
| gbc10 | 0 | 0 | 0.002512 | 0.029926 | 0 | 0.000103 | 0 | 2.02E−05 | 0 | 0.005759 |
| gbc11 | 0 | 0 | 0 | 0.021857 | 4.08E−05 | 0.010324 | 0.000625 | 0 | 0 | 5.01E−05 |
| gbc12 | 0 | 0.001629 | 0.017161 | 0.002238 | 0.010269 | 0.02812 | 0 | 0 | 0.007152 | 0 |
| gbc13 | 0.000509 | 0 | 8.79E−06 | 0.006478 | 0 | 0.01525 | 1.36E−06 | 1.76E−05 | 0.006795 | 0.002196 |
| gbc14 | 0 | 0.000136 | 0.000264 | 0.006717 | 0.004227 | 5.96E−05 | 5.31E−05 | 0.000425 | 0 | 0.000792 |
| gbc15 | 2.70E−05 | 0 | 0 | 0.000798 | 0 | 0.000944 | 8.81E−05 | 0.016653 | 2.42E−07 | 0.002672 |
| gbc16 | 0 | 1.32E−05 | 0.000114 | 0.001156 | 0 | 0.015451 | 5.35E−05 | 0.00021 | 0 | 0.000258 |
| gbc17 | 0 | 0.000861 | 2.34E−05 | 0 | 0 | 0.000146 | 1.02E−05 | 0.000247 | 0.003013 | 5.07E−05 |
| gbc18 | 7.20E−05 | 7.29E−05 | 0 | 0.002564 | 0 | 5.53E−05 | 0 | 0 | 3.85E−05 | 0.005421 |
| gbc19 | 2.50E−05 | 4.19E−07 | 0.000165 | 6.67E−05 | 0.014178 | 0.000697 | 0 | 0 | 0 | 0 |
| gbc20 | 0 | 0.000722 | 4.64E−05 | 2.63E−05 | 0 | 0.000697 | 0 | 0.003086 | 0 | 0.000187 |
| gbc21 | 0 | 0 | 0 | 0.01342 | 2.13E−05 | 0.009249 | 0 | 0.00064 | 0.001935 | 0 |
| gbc22 | 0 | 0 | 0.001417 | 0.000167 | 0.003341 | 0 | 0 | 0.000947 | 0.001989 | 0.000105 |
| gbc23 | 0 | 0 | 0.000172 | 0.000164 | 0 | 1.36E−05 | 0 | 0.000359 | 0.000956 | 0 |
| gbc24 | 0 | 0 | 0.002512 | 0.029926 | 0 | 0.000103 | 0 | 2.02E−05 | 0 | 0.005759 |
| gbc25 | 0 | 0 | 0 | 0.021857 | 4.08E−05 | 0.010324 | 0.000625 | 0 | 0 | 5.01E−05 |
| gbc26 | 0 | 0.001629 | 0.017161 | 0.002238 | 0.010269 | 0.02812 | 0 | 0 | 0.007152 | 0 |
| gbc27 | 0.000509 | 0 | 8.79E−06 | 0.006478 | 0 | 0.01525 | 1.36E−06 | 1.76E−05 | 0.006795 | 0.002196 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | AL137024.1 | AL139095.4 | AL606491.1 | ALG10 | ANGPT1 | ANKRA2 | ANXA11 | AP2B1 | AP2M1 | APOOL |
|---|---|---|---|---|---|---|---|---|---|---|
| rf0 | 7.98E−05 | 0.004862 | 0 | 0.002004 | 0.00033 | 0.004667 | 0 | 0.001358 | 0 | 0.001121 |
| rf1 | 0.001264 | 0 | 0.014191 | 0.002968 | 0.000965 | 0 | 0.007482 | 0 | 0.001497 | 0 |
| rf2 | 0 | 0.003713 | 0.003348 | 0 | 0.009999 | 7.98E−05 | 0.000663 | 0.003416 | 0.0035 | 0.006781 |
| rf3 | 0.000333 | 0.001705 | 0.016522 | 0.003635 | 0.000845 | 0.005537 | 0.00383 | 0.001158 | 0.001447 | 0.004854 |
| rf4 | 0.002785 | 0.000576 | 0.002501 | 0.001493 | 0.000672 | 0.003957 | 0.00332 | 0.0054 | 0.001523 | 0.002947 |
| rf5 | 0.002676 | 0.002911 | 0 | 0.001346 | 0.005682 | 0.006187 | 0.003427 | 0.002415 | 0.000925 | 0.002822 |
| rf6 | 0 | 2.41E−18 | 0.000774 | 0 | 0.000891 | 0.002802 | 0.000351 | 0.001153 | 0 | 0.002142 |
| rf7 | 0.004594 | 0 | 0.000303 | 0.000464 | 0.004163 | 0.000311 | 5.23E−19 | 0 | 0 | 0.000352 |
| rf8 | 0 | 0.001289 | 0.002245 | 0.0008 | 0.003972 | 0 | 0.00194 | 0.002474 | 0.005086 | 0.006956 |
| rf9 | 0 | 0.00152 | 0.003085 | 0 | 0.002684 | 0.002629 | 0.000957 | 0.002381 | 0.003971 | 0.001028 |
| rf10 | 0.003317 | 0.001036 | 0 | 0.000767 | 0.00196 | 2.10E−18 | 0.000527 | 0 | 4.31E−19 | 0 |
| rf11 | 0 | 0 | 0.006015 | 0.000377 | 0.020124 | 0.000394 | 0.006051 | 0.000329 | 0.001426 | 0.001421 |
| rf12 | 0.007321 | 0.001824 | 0.001344 | 0 | 0 | 0 | 0.001059 | 6.34E−18 | 0 | 4.25E−05 |
| rf13 | 0.004951 | 0.00036 | 0 | 0 | 0.004759 | 0 | 0 | 0.001518 | 0.001139 | 0 |
| rf14 | 7.98E−05 | 0.004862 | 0 | 0.002004 | 0.00033 | 0.004667 | 0 | 0.001358 | 0 | 0.001121 |
| rf15 | 0.001264 | 0 | 0.014191 | 0.002968 | 0.000965 | 0 | 0.007482 | 0 | 0.001497 | 0 |
| rf16 | 0 | 0.003713 | 0.003348 | 0 | 0.009999 | 7.98E−05 | 0.000663 | 0.003416 | 0.0035 | 0.006781 |
| rf17 | 0.000333 | 0.001705 | 0.016522 | 0.003635 | 0.000845 | 0.005537 | 0.00383 | 0.001158 | 0.001447 | 0.004854 |
| rf18 | 0.002785 | 0.000576 | 0.002501 | 0.001493 | 0.000672 | 0.003957 | 0.00332 | 0.0054 | 0.001523 | 0.002947 |
| rf19 | 0.002676 | 0.002911 | 0 | 0.001346 | 0.005682 | 0.006187 | 0.003427 | 0.002415 | 0.000925 | 0.002822 |
| rf20 | 0 | 2.41E−18 | 0.000774 | 0 | 0.000891 | 0.002802 | 0.000351 | 0.001153 | 0 | 0.002142 |
| rf21 | 0.004594 | 0 | 0.000303 | 0.000464 | 0.004163 | 0.000311 | 5.23E−19 | 0 | 0 | 0.000352 |
| rf22 | 0 | 0.001289 | 0.002245 | 0.0008 | 0.003972 | 0 | 0.00194 | 0.002474 | 0.005086 | 0.006956 |
| rf23 | 0 | 0.00152 | 0.003085 | 0 | 0.002684 | 0.002629 | 0.000957 | 0.002381 | 0.003971 | 0.001028 |
| rf24 | 0.003317 | 0.001036 | 0 | 0.000767 | 0.00196 | 2.10E−18 | 0.000527 | 0 | 4.31E−19 | 0 |
| rf25 | 0 | 0 | 0.006015 | 0.000377 | 0.020124 | 0.000394 | 0.006051 | 0.000329 | 0.001426 | 0.001421 |
| rf26 | 0.007321 | 0.001824 | 0.001344 | 0 | 0 | 0 | 0.001059 | 6.34E−18 | 0 | 4.25E−05 |
| rf27 | 0.004951 | 0.00036 | 0 | 0 | 0.004759 | 0 | 0 | 0.001518 | 0.001139 | 0 |
| extraTrees0 | 0.003644 | 0.001695 | 0.001708 | 0.002515 | 0.00372 | 0.001657 | 0.001161 | 0.000434 | 0.000864 | 0.00031 |
| extraTrees1 | 0.002078 | 0.000651 | 0.002221 | 0.00065 | 0.004005 | 0.000965 | 0.002975 | 0.001468 | 0.001078 | 0.003376 |
| extraTrees2 | 0.003533 | 0.000733 | 0.003084 | 0.000585 | 0.005726 | 0.001006 | 0.000795 | 0.000529 | 0.001923 | 0.000384 |
| extraTrees3 | 0.001061 | 0.003671 | 0.005213 | 0.003306 | 0.003573 | 0.002607 | 0.000498 | 0.000581 | 0.002456 | 0.000656 |
| extraTrees4 | 0.004378 | 0.00137 | 0.002688 | 0.001779 | 0.004859 | 0 | 0.000523 | 0.000674 | 1.57E−17 | 0.000684 |
| extraTrees5 | 0.002009 | 0.001299 | 0.002484 | 0.000537 | 0.002548 | 0.000816 | 0.001333 | 0.000866 | 0.000627 | 0.00315 |
| extraTrees6 | 0.000524 | 0.001424 | 0.00602 | 0.001743 | 0.006805 | 4.10E−18 | 0.000434 | 0.000846 | 0.001059 | 0.001259 |
| extraTrees7 | 0.00116 | 0.002809 | 0.002047 | 0.001851 | 0.005169 | 0.001029 | 0.002738 | 0.00028 | 0.004568 | 0.001182 |
| extraTrees8 | 0.001944 | 0.000287 | 0.001536 | 0.00239 | 0.004878 | 0.000669 | 0.004732 | 0.001523 | 0.000828 | 0.00148 |
| extraTrees9 | 0.001214 | 0.001 | 0.005927 | 0.002093 | 0.004853 | 0.001813 | 0.004895 | 0.000473 | 0.002069 | 0.006474 |
| extraTrees10 | 0.00139 | 0.00555 | 0.001478 | 0.002048 | 0.003121 | 0.001997 | 0.001733 | 0.000267 | 0.001148 | 0.001472 |
| extraTrees11 | 0.006192 | 0.001841 | 0.006542 | 0.000388 | 0.002823 | 0.000371 | 0.001417 | 2.48E−18 | 0.000609 | 0.000747 |
| extraTrees12 | 0.002899 | 0.001224 | 0.002443 | 0.002705 | 0.001352 | 0.000253 | 0.00116 | 0 | 0.000201 | 0.00254 |
| extraTrees13 | 0.001911 | 0.001798 | 0.003507 | 0.001771 | 0.002575 | 0.000916 | 0.008288 | 0.000822 | 0 | 0.002587 |
| extraTrees14 | 0.003644 | 0.001695 | 0.001708 | 0.002515 | 0.00372 | 0.001657 | 0.001161 | 0.000434 | 0.000864 | 0.00031 |
| extraTrees15 | 0.002078 | 0.000651 | 0.002221 | 0.00065 | 0.004005 | 0.000965 | 0.002975 | 0.001468 | 0.001078 | 0.003376 |
| extraTrees16 | 0.003533 | 0.000733 | 0.003084 | 0.000585 | 0.005726 | 0.001006 | 0.000795 | 0.000529 | 0.001923 | 0.000384 |
| extraTrees17 | 0.001061 | 0.003671 | 0.005213 | 0.003306 | 0.003573 | 0.002607 | 0.000498 | 0.000581 | 0.002456 | 0.000656 |
| extraTrees18 | 0.004378 | 0.00137 | 0.002688 | 0.001779 | 0.004859 | 0 | 0.000523 | 0.000674 | 1.57E−17 | 0.000684 |
| extraTrees19 | 0.002009 | 0.001299 | 0.002484 | 0.000537 | 0.002548 | 0.000816 | 0.001333 | 0.000866 | 0.000627 | 0.00315 |
| extraTrees20 | 0.000524 | 0.001424 | 0.00602 | 0.001743 | 0.006805 | 4.10E−18 | 0.000434 | 0.000846 | 0.001059 | 0.001259 |
| extraTrees21 | 0.00116 | 0.002809 | 0.002047 | 0.001851 | 0.005169 | 0.001029 | 0.002738 | 0.00028 | 0.004568 | 0.001182 |
| extraTrees22 | 0.001944 | 0.000287 | 0.001536 | 0.00239 | 0.004878 | 0.000669 | 0.004732 | 0.001523 | 0.000828 | 0.00148 |
| extraTrees23 | 0.001214 | 0.001 | 0.005927 | 0.002093 | 0.004853 | 0.001813 | 0.004895 | 0.000473 | 0.002069 | 0.006474 |
| extraTrees24 | 0.00139 | 0.00555 | 0.001478 | 0.002048 | 0.003121 | 0.001997 | 0.001733 | 0.000267 | 0.001148 | 0.001472 |
| extraTrees25 | 0.006192 | 0.001841 | 0.006542 | 0.000388 | 0.002823 | 0.000371 | 0.001417 | 2.48E−18 | 0.000609 | 0.000747 |
| extraTrees26 | 0.002899 | 0.001224 | 0.002443 | 0.002705 | 0.001352 | 0.000253 | 0.00116 | 0 | 0.000201 | 0.00254 |
| extraTrees27 | 0.001911 | 0.001798 | 0.003507 | 0.001771 | 0.002575 | 0.000916 | 0.008288 | 0.000822 | 0 | 0.002587 |
| gbc0 | 2.24E−05 | 0.002717 | 0 | 0 | 0.000226 | 0 | 0 | 0 | 0 | 0.000266 |
| gbc1 | 0.009132 | 8.16E−05 | 0.015721 | 0.000424 | 0 | 0.003604 | 0 | 0.000245 | 0 | 0 |
| gbc2 | 0 | 0 | 0.000802 | 0.016594 | 5.71E−05 | 3.69E−05 | 0 | 1.57E−05 | 0.000335 | 6.01E−05 |
| gbc3 | 0.000287 | 0 | 2.38E−05 | 0 | 0.000889 | 0 | 0 | 0 | 0 | 0.001341 |
| gbc4 | 0.000344 | 0.002854 | 0.000732 | 0.004532 | 0.000217 | 0.037031 | 0 | 5.40E−05 | 0 | 0 |
| gbc5 | 0.002486 | 0 | 0.000344 | 0.030029 | 0 | 0 | 0 | 1.59E−05 | 0 | 0 |
| gbc6 | 0.00525 | 0.012592 | 0 | 1.65E−05 | 9.44E−05 | 4.98E−06 | 0 | 0 | 0 | 1.03E−05 |
| gbc7 | 0.000276 | 2.92E−05 | 0.000194 | 0 | 4.66E−05 | 0 | 0 | 0 | 0 | 0 |
| gbc8 | 0.001947 | 0.000135 | 0.000591 | 0.000102 | 0.009315 | 0 | 6.03E−05 | 0 | 0 | 0 |
| gbc9 | 0.013331 | 0 | 0 | 0 | 9.36E−06 | 0.00419 | 0.014802 | 0 | 0.001356 | 0 |
| gbc10 | 0.003393 | 0 | 0 | 9.45E−05 | 0 | 0 | 9.98E−05 | 2.55E−05 | 0 | 0 |
| gbc11 | 0 | 0.000257 | 1.24E−05 | 0.000688 | 0 | 0 | 0.000361 | 0.000404 | 0 | 0.000291 |
| gbc12 | 0 | 8.59E−06 | 0.00102 | 0.002719 | 0 | 0 | 0 | 0 | 0 | 0.001679 |
| gbc13 | 0.017689 | 0.000451 | 6.17E−06 | 1.02E−05 | 3.41E−06 | 0.000282 | 0 | 0 | 0 | 0 |
| gbc14 | 2.24E−05 | 0.002717 | 0 | 0 | 0.000226 | 0 | 0 | 0 | 0 | 0.000266 |
| gbc15 | 0.009132 | 8.16E−05 | 0.015721 | 0.000424 | 0 | 0.003604 | 0 | 0.000245 | 0 | 0 |
| gbc16 | 0 | 0 | 0.000802 | 0.016594 | 5.71E−05 | 3.69E−05 | 0 | 1.57E−05 | 0.000335 | 6.01E−05 |
| gbc17 | 0.000287 | 0 | 2.38E−05 | 0 | 0.000889 | 0 | 0 | 0 | 0 | 0.001341 |
| gbc18 | 0.000344 | 0.002854 | 0.000732 | 0.004532 | 0.000217 | 0.037031 | 0 | 5.40E−05 | 0 | 0 |
| gbc19 | 0.002486 | 0 | 0.000344 | 0.030029 | 0 | 0 | 0 | 1.59E−05 | 0 | 0 |
| gbc20 | 0.00525 | 0.012592 | 0 | 1.65E−05 | 9.44E−05 | 4.98E−06 | 0 | 0 | 0 | 1.03E−05 |
| gbc21 | 0.000276 | 2.92E−05 | 0.000194 | 0 | 4.66E−05 | 0 | 0 | 0 | 0 | 0 |
| gbc22 | 0.001947 | 0.000135 | 0.000591 | 0.000102 | 0.009315 | 0 | 6.03E−05 | 0 | 0 | 0 |
| gbc23 | 0.013331 | 0 | 0 | 0 | 9.36E−06 | 0.00419 | 0.014802 | 0 | 0.001356 | 0 |
| gbc24 | 0.003393 | 0 | 0 | 9.45E−05 | 0 | 0 | 9.98E−05 | 2.55E−05 | 0 | 0 |
| gbc25 | 0 | 0.000257 | 1.24E−05 | 0.000688 | 0 | 0 | 0.000361 | 0.000404 | 0 | 0.000291 |
| gbc26 | 0 | 8.59E−06 | 0.00102 | 0.002719 | 0 | 0 | 0 | 0 | 0 | 0.001679 |
| gbc27 | 0.017689 | 0.000451 | 6.17E−06 | 1.02E−05 | 3.41E−06 | 0.000282 | 0 | 0 | 0 | 0 |
| ada0 | 0 | 0 | 0.02 | 0 | 0 | 0 | 0 | 0 | 0 | 0 |
| ada1 | 0 | 0 | 0.02 | 0 | 0 | 0 | 0 | 0 | 0 | 0 |
| ada2 | 0 | 0 | 0.02 | 0 | 0 | 0 | 0 | 0 | 0 | 0 |
| ada3 | 0 | 0 | 0.02 | 0 | 0 | 0 | 0 | 0 | 0 | 0 |
| ada4 | 0 | 0 | 0.02 | 0 | 0 | 0 | 0 | 0 | 0 | 0 |
| ada5 | 0 | 0 | 0.02 | 0 | 0 | 0 | 0 | 0 | 0 | 0 |
| ada6 | 0 | 0 | 0.02 | 0 | 0 | 0 | 0 | 0 | 0 | 0 |
| ada7 | 0 | 0 | 0.02 | 0 | 0 | 0 | 0 | 0 | 0 | 0 |
| ada8 | 0 | 0 | 0.02 | 0 | 0 | 0 | 0 | 0 | 0 | 0 |
| ada9 | 0 | 0 | 0.02 | 0 | 0 | 0 | 0 | 0 | 0 | 0 |
| ada10 | 0 | 0 | 0.02 | 0 | 0 | 0 | 0 | 0 | 0 | 0 |
| ada11 | 0 | 0 | 0.02 | 0 | 0 | 0 | 0 | 0 | 0 | 0 |
| ada12 | 0 | 0 | 0.02 | 0 | 0 | 0 | 0 | 0 | 0 | 0 |
| ada13 | 0 | 0 | 0.02 | 0 | 0 | 0 | 0 | 0 | 0 | 0 |
| ada14 | 0 | 0 | 0.02 | 0 | 0 | 0 | 0 | 0 | 0 | 0 |
| ada15 | 0 | 0 | 0.02 | 0 | 0 | 0 | 0 | 0 | 0 | 0 |
| ada16 | 0 | 0 | 0.02 | 0 | 0 | 0 | 0 | 0 | 0 | 0 |
| ada17 | 0 | 0 | 0.02 | 0 | 0 | 0 | 0 | 0 | 0 | 0 |
| ada18 | 0 | 0 | 0.02 | 0 | 0 | 0 | 0 | 0 | 0 | 0 |
| ada19 | 0 | 0 | 0.02 | 0 | 0 | 0 | 0 | 0 | 0 | 0 |
| ada20 | 0 | 0 | 0.02 | 0 | 0 | 0 | 0 | 0 | 0 | 0 |
| ada21 | 0 | 0 | 0.02 | 0 | 0 | 0 | 0 | 0 | 0 | 0 |
| ada22 | 0 | 0 | 0.02 | 0 | 0 | 0 | 0 | 0 | 0 | 0 |
| ada23 | 0 | 0 | 0.02 | 0 | 0 | 0 | 0 | 0 | 0 | 0 |
| ada24 | 0 | 0 | 0.02 | 0 | 0 | 0 | 0 | 0 | 0 | 0 |
| ada25 | 0 | 0 | 0.02 | 0 | 0 | 0 | 0 | 0 | 0 | 0 |
| ada26 | 0 | 0 | 0.02 | 0 | 0 | 0 | 0 | 0 | 0 | 0 |
| ada27 | 0 | 0 | 0.02 | 0 | 0 | 0 | 0 | 0 | 0 | 0 |

| Gene | ARF1 | ARFGEF1 | ARHGAP5 | ARHGDIB | ARHGEF12 | ARID1A | ARL1 | ARPC1B | ASS1P1 | ATF4 |
|---|---|---|---|---|---|---|---|---|---|---|
| rf0 | 0 | 6.07E−18 | 0.006363 | 0.004007 | 0.001691 | 0.001794 | 0 | 7.87E−05 | 0.00038 | 0.006477 |
| rf1 | 0.000377 | 0.000863 | 0.000349 | 0 | 8.17E−05 | 0 | 0.010868 | 0.004385 | 0.001024 | 0.001721 |
| rf2 | 0.005519 | 0.001178 | 0.004314 | 0.002458 | 0.002969 | 0.008119 | 0 | 0.001756 | 0 | 0.006387 |
| rf3 | 0.003435 | 0.000459 | 0.002327 | 0.005138 | 0 | 4.96E−19 | 0 | 0 | 0.006777 | 0.000818 |
| rf4 | 0 | 0 | 0.001051 | 0.006797 | 0.001573 | 0 | 1.45E−17 | 0 | 0 | 0 |
| rf5 | 0.001177 | 0.000604 | 0.006429 | 0.011339 | 0.002839 | 0 | 0.003959 | 0.004096 | 0.00042 | 0 |
| rf6 | 0 | 0 | 0.002397 | 0.005916 | 0.000357 | 0 | 0 | 0.002462 | 0 | 0.004771 |
| rf7 | 0.001425 | 0 | 0 | 0.000366 | 0.006674 | 0 | 0 | 0.006744 | 0 | 0 |
| rf8 | 0.001358 | 0.001221 | 0.004792 | 0 | 5.58E−19 | 0 | 0.003927 | 0 | 0.000969 | 0.004548 |
| rf9 | 0.000627 | 0.002026 | 0.007791 | 0.009007 | 0.003759 | 0 | 0.00403 | 3.11E−18 | 0.00276 | 0 |
| rf10 | 0.008111 | 0.001309 | 0.005603 | 0.01116 | 1.27E−18 | 0 | 0.000387 | 0.000389 | 0.001038 | 0.002182 |
| rf11 | 0.001023 | 0 | 0 | 0 | 0.000144 | 0.000753 | 0 | 0 | 0.00138 | 0 |
| rf12 | 0 | 0.003343 | 0.002348 | 0 | 0.000288 | 0.00801 | 0.003184 | 0.002873 | 0 | 0.005585 |
| rf13 | 0 | 0.00631 | 0.007124 | 0.001689 | 0 | 0 | 0.002477 | 0.001381 | 0.001401 | 0.002528 |
| rf14 | 0 | 6.07E−18 | 0.006363 | 0.004007 | 0.001691 | 0.001794 | 0 | 7.87E−05 | 0.00038 | 0.006477 |
| rf15 | 0.000377 | 0.000863 | 0.000349 | 0 | 8.17E−05 | 0 | 0.010868 | 0.004385 | 0.001024 | 0.001721 |
| rf16 | 0.005519 | 0.001178 | 0.004314 | 0.002458 | 0.002969 | 0.008119 | 0 | 0.001756 | 0 | 0.006387 |
| rf17 | 0.003435 | 0.000459 | 0.002327 | 0.005138 | 0 | 4.96E−19 | 0 | 0 | 0.006777 | 0.000818 |
| rf18 | 0 | 0 | 0.001051 | 0.006797 | 0.001573 | 0 | 1.45E−17 | 0 | 0 | 0 |
| rf19 | 0.001177 | 0.000604 | 0.006429 | 0.011339 | 0.002839 | 0 | 0.003959 | 0.004096 | 0.00042 | 0 |
| rf20 | 0 | 0 | 0.002397 | 0.005916 | 0.000357 | 0 | 0 | 0.002462 | 0 | 0.004771 |
| rf21 | 0.001425 | 0 | 0 | 0.000366 | 0.006674 | 0 | 0 | 0.006744 | 0 | 0 |
| rf22 | 0.001358 | 0.001221 | 0.004792 | 0 | 5.58E−19 | 0 | 0.003927 | 0 | 0.000969 | 0.004548 |
| rf23 | 0.000627 | 0.002026 | 0.007791 | 0.009007 | 0.003759 | 0 | 0.00403 | 3.11E−18 | 0.00276 | 0 |
| rf24 | 0.008111 | 0.001309 | 0.005603 | 0.01116 | 1.27E−18 | 0 | 0.000387 | 0.000389 | 0.001038 | 0.002182 |
| rf25 | 0.001023 | 0 | 0 | 0 | 0.000144 | 0.000753 | 0 | 0 | 0.00138 | 0 |
| rf26 | 0 | 0.003343 | 0.002348 | 0 | 0.000288 | 0.00801 | 0.003184 | 0.002873 | 0 | 0.005585 |
| rf27 | 0 | 0.00631 | 0.007124 | 0.001689 | 0 | 0 | 0.002477 | 0.001381 | 0.001401 | 0.002528 |
| extraTrees0 | 0.001215 | 0.002029 | 0.006633 | 0.000952 | 0.000434 | 0.003658 | 0.000738 | 0.00234 | 0.001285 | 0.000961 |
| extraTrees1 | 0.002536 | 0.002186 | 0.004072 | 0.001204 | 0.002479 | 0.001121 | 0.000685 | 0.001407 | 0.000757 | 0.000476 |
| extraTrees2 | 0.000987 | 2.73E−19 | 0.00761 | 0.001089 | 0.003521 | 0.000428 | 0.001032 | 0.002016 | 0.000577 | 0.000604 |
| extraTrees3 | 0.001107 | 0.000533 | 0.005309 | 0.007 | 0.002385 | 0.000281 | 0.003783 | 0.001337 | 0.001068 | 0.000476 |
| extraTrees4 | 0.00163 | 0.002585 | 0.001404 | 0.013863 | 0.000756 | 0.000238 | 0.001565 | 0.002105 | 0.00037 | 0.000238 |
| extraTrees5 | 0.000492 | 0.000668 | 0.003864 | 0.000597 | 0.000696 | 0.004098 | 0.001326 | 0.00028 | 0 | 0.001459 |
| extraTrees6 | 0.000857 | 0.000518 | 0.003121 | 0.002149 | 0.001299 | 0.000476 | 0.002972 | 0.00028 | 0.002161 | 0.000427 |
| extraTrees7 | 0.002592 | 0.001438 | 0.005974 | 0.001637 | 0.003906 | 0.002106 | 0.002517 | 0.00096 | 0.002961 | 0.00141 |
| extraTrees8 | 0.000238 | 0.002193 | 0.00494 | 0.002194 | 6.37E−05 | 1.69E−18 | 0.002461 | 0.001622 | 0.000268 | 0.001103 |
| extraTrees9 | 0.00025 | 0.002166 | 0.004643 | 0.005491 | 0.005476 | 0.001902 | 0.000872 | 0.005999 | 0.001091 | 0 |
| extraTrees10 | 0.002756 | 0.001405 | 0.004347 | 0.003913 | 0.002145 | 0.000921 | 0.001276 | 0.001933 | 0.000949 | 0.000737 |
| extraTrees11 | 0.002967 | 0.001717 | 0.003471 | 0.005845 | 0.000792 | 0.004214 | 0.002702 | 0.001912 | 0.001047 | 0.000939 |
| extraTrees12 | 0.002581 | 0.002023 | 0.006831 | 0.002828 | 0.001158 | 0.003397 | 0.001813 | 0.000238 | 0.001762 | 0.001562 |
| extraTrees13 | 0.001089 | 0.000492 | 0.005351 | 0.002824 | 0.000623 | 0.001704 | 0.000547 | 0.003222 | 0.001165 | 0.004332 |
| extraTrees14 | 0.001215 | 0.002029 | 0.006633 | 0.000952 | 0.000434 | 0.003658 | 0.000738 | 0.00234 | 0.001285 | 0.000961 |
| extraTrees15 | 0.002536 | 0.002186 | 0.004072 | 0.001204 | 0.002479 | 0.001121 | 0.000685 | 0.001407 | 0.000757 | 0.000476 |
| extraTrees16 | 0.000987 | 2.73E−19 | 0.00761 | 0.001089 | 0.003521 | 0.000428 | 0.001032 | 0.002016 | 0.000577 | 0.000604 |
| extraTrees17 | 0.001107 | 0.000533 | 0.005309 | 0.007 | 0.002385 | 0.000281 | 0.003783 | 0.001337 | 0.001068 | 0.000476 |
| extraTrees18 | 0.00163 | 0.002585 | 0.001404 | 0.013863 | 0.000756 | 0.000238 | 0.001565 | 0.002105 | 0.00037 | 0.000238 |
| extraTrees19 | 0.000492 | 0.000668 | 0.003864 | 0.000597 | 0.000696 | 0.004098 | 0.001326 | 0.00028 | 0 | 0.001459 |
| extraTrees20 | 0.000857 | 0.000518 | 0.003121 | 0.002149 | 0.001299 | 0.000476 | 0.002972 | 0.00028 | 0.002161 | 0.000427 |
| extraTrees21 | 0.002592 | 0.001438 | 0.005974 | 0.001637 | 0.003906 | 0.002106 | 0.002517 | 0.00096 | 0.002961 | 0.00141 |
| extraTrees22 | 0.000238 | 0.002193 | 0.00494 | 0.002194 | 6.37E−05 | 1.69E−18 | 0.002461 | 0.001622 | 0.000268 | 0.001103 |
| extraTrees23 | 0.00025 | 0.002166 | 0.004643 | 0.005491 | 0.005476 | 0.001902 | 0.000872 | 0.005999 | 0.001091 | 0 |
| extraTrees24 | 0.002756 | 0.001405 | 0.004347 | 0.003913 | 0.002145 | 0.000921 | 0.001276 | 0.001933 | 0.000949 | 0.000737 |
| extraTrees25 | 0.002967 | 0.001717 | 0.003471 | 0.005845 | 0.000792 | 0.004214 | 0.002702 | 0.001912 | 0.001047 | 0.000939 |
| extraTrees26 | 0.002581 | 0.002023 | 0.006831 | 0.002828 | 0.001158 | 0.003397 | 0.001813 | 0.000238 | 0.001762 | 0.001562 |
| extraTrees27 | 0.001089 | 0.000492 | 0.005351 | 0.002824 | 0.000623 | 0.001704 | 0.000547 | 0.003222 | 0.001165 | 0.004332 |
| gbc0 | 0.000306 | 0 | 0.005512 | 0 | 0.002234 | 0 | 0 | 0.001279 | 0 | 0 |
| gbc1 | 0 | 3.53E−05 | 7.34E−05 | 0.000119 | 0 | 0.0001 | 0 | 0.008932 | 0 | 1.80E−06 |
| gbc2 | 0 | 0 | 0 | 2.66E−07 | 0 | 0.018332 | 0 | 0.006367 | 6.48E−05 | 0 |
| gbc3 | 3.88E−05 | 0.000363 | 0 | 0.00085 | 0.002484 | 0.000583 | 0 | 0 | 0 | 3.03E−05 |
| gbc4 | 0 | 0 | 0 | 0.003423 | 0.003404 | 0.000478 | 0 | 0.000182 | 0.000129 | 0 |
| gbc5 | 0 | 0 | 0.025164 | 0 | 0 | 0.018961 | 7.66E−06 | 0 | 6.79E−05 | 0 |
| gbc6 | 1.91E−05 | 0.001597 | 0.000343 | 0 | 0 | 0 | 2.15E−07 | 0 | 0 | 0 |
| gbc7 | 0 | 6.82E−05 | 0 | 0 | 0.003654 | 0 | 0.009642 | 0 | 0.000644 | 0 |
| gbc8 | 0 | 0 | 0 | 0.000122 | 0 | 0.001323 | 0 | 0 | 0.002929 | 0 |
| gbc9 | 0 | 0.000571 | 0 | 0 | 0 | 0.001304 | 3.18E−05 | 0.002258 | 0 | 0 |
| gbc10 | 0 | 0 | 0.014602 | 0.044654 | 6.77E−05 | 0.011243 | 0.014347 | 0 | 0 | 0 |
| gbc11 | 0.022072 | 0 | 0.038984 | 2.92E−05 | 0 | 0.017669 | 0.025309 | 0 | 0.000602 | 0.000386 |
| gbc12 | 0.000347 | 0 | 0.003437 | 0 | 0.000143 | 0 | 0 | 1.11E−05 | 0.000101 | 4.42E−06 |
| gbc13 | 0.000214 | 0 | 0.032892 | 0.018013 | 0 | 0.003426 | 0.000374 | 0.000764 | 0 | 7.45E−05 |
| gbc14 | 0.000306 | 0 | 0.005512 | 0 | 0.002234 | 0 | 0 | 0.001279 | 0 | 0 |
| gbc15 | 0 | 3.53E−05 | 7.34E−05 | 0.000119 | 0 | 0.0001 | 0 | 0.008932 | 0 | 1.80E−06 |
| gbc16 | 0 | 0 | 0 | 2.66E−07 | 0 | 0.018332 | 0 | 0.006367 | 6.48E−05 | 0 |
| gbc17 | 3.88E−05 | 0.000363 | 0 | 0.00085 | 0.002484 | 0.000583 | 0 | 0 | 0 | 3.03E−05 |
| gbc18 | 0 | 0 | 0 | 0.003423 | 0.003404 | 0.000478 | 0 | 0.000182 | 0.000129 | 0 |
| gbc19 | 0 | 0 | 0.025164 | 0 | 0 | 0.018961 | 7.66E−06 | 0 | 6.79E−05 | 0 |
| gbc20 | 1.91E−05 | 0.001597 | 0.000343 | 0 | 0 | 0 | 2.15E−07 | 0 | 0 | 0 |
| gbc21 | 0 | 6.82E−05 | 0 | 0 | 0.003654 | 0 | 0.009642 | 0 | 0.000644 | 0 |
| gbc22 | 0 | 0 | 0 | 0.000122 | 0 | 0.001323 | 0 | 0 | 0.002929 | 0 |
| gbc23 | 0 | 0.000571 | 0 | 0 | 0 | 0.001304 | 3.18E−05 | 0.002258 | 0 | 0 |
| gbc24 | 0 | 0 | 0.014602 | 0.044654 | 6.77E−05 | 0.011243 | 0.014347 | 0 | 0 | 0 |
| gbc25 | 0.022072 | 0 | 0.038984 | 2.92E−05 | 0 | 0.017669 | 0.025309 | 0 | 0.000602 | 0.000386 |
| gbc26 | 0.000347 | 0 | 0.003437 | 0 | 0.000143 | 0 | 0 | 1.11E−05 | 0.000101 | 4.42E−06 |
| gbc27 | 0.000214 | 0 | 0.032892 | 0.018013 | 0 | 0.003426 | 0.000374 | 0.000764 | 0 | 7.45E−05 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | ATF4P4 | ATF7IP | ATG2B | ATP6V0A2 | ATXN7 | ATXN7L3 | BAG2 | BAG4 | BANK1 | BAP1 |
|---|---|---|---|---|---|---|---|---|---|---|
| rf0 | 0 | 0.001083 | 0.000293 | 0.001511 | 0.000645 | 0.000971 | 0 | 5.25E−19 | 0.003155 | 0 |
| rf1 | 0 | 0.001603 | 0.001508 | 0 | 0.009165 | 0.003439 | 0 | 0 | 0.006029 | 0 |
| rf2 | 6.86E−19 | 0.002173 | 0 | 0.001096 | 5.25E−19 | 0.00197 | 0.00079 | 0.001413 | 0.01228 | 3.66E−18 |
| rf3 | 0 | 4.53E−18 | 0 | 0 | 0 | 0.001717 | 0.000275 | 3.88E−18 | 0.010854 | 0.000703 |
| rf4 | 0.001902 | 0.003899 | 0.001897 | 0 | 0.005867 | 0.005042 | 0.001628 | 0.00302 | 0.005608 | 0.001391 |
| rf5 | 0.001313 | 0.00136 | 0.000893 | 0.002505 | 0 | 0.000615 | 0.001483 | 0.000953 | 0.010079 | 0.001182 |
| rf6 | 0.000947 | 0.000358 | 0.000372 | 0.004948 | 0.000657 | 0 | 0 | 0 | 0.003844 | 0.000604 |
| rf7 | 0.000959 | 0.000713 | 0 | 0.002197 | 0 | 0.003458 | 4.24E−05 | 0.000416 | 0.006951 | 0.001311 |
| rf8 | 0 | 0.002056 | 0 | 0.001463 | 0.000351 | 0 | 0.001318 | 0 | 0.006928 | 0 |
| rf9 | 0.00066 | 0.004644 | 0.000311 | 0.002818 | 0 | 0.004069 | 0 | 0.00319 | 0.000733 | 0.002326 |
| rf10 | 0.000925 | 0.003486 | 0 | 0.002359 | 0.001525 | 0.003803 | 0 | 0 | 0.008143 | 2.12E−18 |
| rf11 | 0 | 0.007518 | 0.001439 | 0.000317 | 0 | 1.57E−18 | 0 | 0.000973 | 0.002946 | 0.000976 |
| rf12 | 0 | 0.001057 | 0.004893 | 0.002636 | 0 | 0.005556 | 0.000748 | 0.000969 | 0.012253 | 0.002061 |
| rf13 | 0.001453 | 0 | 0.000693 | 0.000347 | 1.24E−18 | 0.001454 | 1.61E−18 | 0 | 0.003387 | 0.000391 |
| rf14 | 0 | 0.001083 | 0.000293 | 0.001511 | 0.000645 | 0.000971 | 0 | 5.25E−19 | 0.003155 | 0 |
| rf15 | 0 | 0.001603 | 0.001508 | 0 | 0.009165 | 0.003439 | 0 | 0 | 0.006029 | 0 |
| rf16 | 6.86E−19 | 0.002173 | 0 | 0.001096 | 5.25E−19 | 0.00197 | 0.00079 | 0.001413 | 0.01228 | 3.66E−18 |
| rf17 | 0 | 4.53E−18 | 0 | 0 | 0 | 0.001717 | 0.000275 | 3.88E−18 | 0.010854 | 0.000703 |
| rf18 | 0.001902 | 0.003899 | 0.001897 | 0 | 0.005867 | 0.005042 | 0.001628 | 0.00302 | 0.005608 | 0.001391 |
| rf19 | 0.001313 | 0.00136 | 0.000893 | 0.002505 | 0 | 0.000615 | 0.001483 | 0.000953 | 0.010079 | 0.001182 |
| rf20 | 0.000947 | 0.000358 | 0.000372 | 0.004948 | 0.000657 | 0 | 0 | 0 | 0.003844 | 0.000604 |
| rf21 | 0.000959 | 0.000713 | 0 | 0.002197 | 0 | 0.003458 | 4.24E−05 | 0.000416 | 0.006951 | 0.001311 |
| rf22 | 0 | 0.002056 | 0 | 0.001463 | 0.000351 | 0 | 0.001318 | 0 | 0.006928 | 0 |
| rf23 | 0.00066 | 0.004644 | 0.000311 | 0.002818 | 0 | 0.004069 | 0 | 0.00319 | 0.000733 | 0.002326 |
| rf24 | 0.000925 | 0.003486 | 0 | 0.002359 | 0.001525 | 0.003803 | 0 | 0 | 0.008143 | 2.12E−18 |
| rf25 | 0 | 0.007518 | 0.001439 | 0.000317 | 0 | 1.57E−18 | 0 | 0.000973 | 0.002946 | 0.000976 |
| rf26 | 0 | 0.001057 | 0.004893 | 0.002636 | 0 | 0.005556 | 0.000748 | 0.000969 | 0.012253 | 0.002061 |
| rf27 | 0.001453 | 0 | 0.000693 | 0.000347 | 1.24E−18 | 0.001454 | 1.61E−18 | 0 | 0.003387 | 0.000391 |
| extraTrees0 | 0.002851 | 0.000535 | 0.000671 | 0.000666 | 0.000693 | 0.001815 | 0.001281 | 0.001582 | 0.014315 | 0.001667 |
| extraTrees1 | 0.002616 | 0.001279 | 0.001352 | 0.002321 | 0.003121 | 0.003043 | 0.001488 | 0.001031 | 0.002641 | 7.48E−05 |
| extraTrees2 | 1.06E−18 | 0.002629 | 0.00318 | 0.000834 | 0.000971 | 0.008636 | 0.001973 | 0.002028 | 0.006284 | 0.000253 |
| extraTrees3 | 0.002374 | 0.001031 | 1.47E−18 | 0.001948 | 0.001094 | 0.000544 | 0.000878 | 0.000841 | 0.008137 | 0.000268 |
| extraTrees4 | 0.002569 | 0.000758 | 0.002263 | 0.003586 | 0.002825 | 0.00139 | 0.002521 | 0.001407 | 0.008768 | 0.001349 |
| extraTrees5 | 0.000557 | 0.000676 | 0.003082 | 0.001874 | 0.000529 | 0.000779 | 0.00188 | 0.000456 | 0.006263 | 0.001218 |
| extraTrees6 | 0.00214 | 0.002306 | 0.000763 | 0.001349 | 0.001109 | 9.56E−18 | 0 | 0.004287 | 0.009016 | 0.00281 |
| extraTrees7 | 0.002094 | 0.002058 | 0.001245 | 0.001061 | 2.66E−18 | 0.003927 | 0.000744 | 0.000799 | 0.017784 | 0.001679 |
| extraTrees8 | 0.002788 | 0.002295 | 0.001291 | 0.00087 | 0.002184 | 0.003025 | 0.002181 | 0.000912 | 0.009624 | 0.000671 |
| extraTrees9 | 0.000492 | 0.001625 | 0.0011 | 0.001015 | 1.33E−17 | 0.000688 | 0.000238 | 0.000668 | 0.009885 | 0.000525 |
| extraTrees10 | 0.002288 | 0.001381 | 8.07E−05 | 0.000234 | 0.00214 | 0.001272 | 0.000396 | 0.000238 | 0.008392 | 0.001374 |
| extraTrees11 | 0.002149 | 0.000387 | 0.002062 | 0.00316 | 0.001369 | 0.002526 | 0.003353 | 0 | 0.00612 | 0.001661 |
| extraTrees12 | 0.000293 | 0.001806 | 0.001843 | 0.001119 | 0.001804 | 0.001099 | 0.000238 | 4.82E−18 | 0.012979 | 0.001591 |
| extraTrees13 | 0.00351 | 0.002028 | 0.001779 | 0.001509 | 0.001249 | 0.002269 | 0 | 0.00056 | 0.011471 | 0.000824 |
| extraTrees14 | 0.002851 | 0.000535 | 0.000671 | 0.000666 | 0.000693 | 0.001815 | 0.001281 | 0.001582 | 0.014315 | 0.001667 |
| extraTrees15 | 0.002616 | 0.001279 | 0.001352 | 0.002321 | 0.003121 | 0.003043 | 0.001488 | 0.001031 | 0.002641 | 7.48E−05 |
| extraTrees16 | 1.06E−18 | 0.002629 | 0.00318 | 0.000834 | 0.000971 | 0.008636 | 0.001973 | 0.002028 | 0.006284 | 0.000253 |
| extraTrees17 | 0.002374 | 0.001031 | 1.47E−18 | 0.001948 | 0.001094 | 0.000544 | 0.000878 | 0.000841 | 0.008137 | 0.000268 |
| extraTrees18 | 0.002569 | 0.000758 | 0.002263 | 0.003586 | 0.002825 | 0.00139 | 0.002521 | 0.001407 | 0.008768 | 0.001349 |
| extraTrees19 | 0.000557 | 0.000676 | 0.003082 | 0.001874 | 0.000529 | 0.000779 | 0.00188 | 0.000456 | 0.006263 | 0.001218 |
| extraTrees20 | 0.00214 | 0.002306 | 0.000763 | 0.001349 | 0.001109 | 9.56E−18 | 0 | 0.004287 | 0.009016 | 0.00281 |
| extraTrees21 | 0.002094 | 0.002058 | 0.001245 | 0.001061 | 2.66E−18 | 0.003927 | 0.000744 | 0.000799 | 0.017784 | 0.001679 |
| extraTrees22 | 0.002788 | 0.002295 | 0.001291 | 0.00087 | 0.002184 | 0.003025 | 0.002181 | 0.000912 | 0.009624 | 0.000671 |
| extraTrees23 | 0.000492 | 0.001625 | 0.0011 | 0.001015 | 1.33E−17 | 0.000688 | 0.000238 | 0.000668 | 0.009885 | 0.000525 |
| extraTrees24 | 0.002288 | 0.001381 | 8.07E−05 | 0.000234 | 0.00214 | 0.001272 | 0.000396 | 0.000238 | 0.008392 | 0.001374 |
| extraTrees25 | 0.002149 | 0.000387 | 0.002062 | 0.00316 | 0.001369 | 0.002526 | 0.003353 | 0 | 0.00612 | 0.001661 |
| extraTrees26 | 0.000293 | 0.001806 | 0.001843 | 0.001119 | 0.001804 | 0.001099 | 0.000238 | 4.82E−18 | 0.012979 | 0.001591 |
| extraTrees27 | 0.00351 | 0.002028 | 0.001779 | 0.001509 | 0.001249 | 0.002269 | 0 | 0.00056 | 0.011471 | 0.000824 |
| gbc0 | 0 | 8.31E−07 | 0.002032 | 0 | 0 | 0 | 0 | 0 | 0.000102 | 1.21E−05 |
| gbc1 | 0 | 0 | 0.001315 | 5.23E−05 | 0 | 0 | 0 | 0 | 0.008454 | 0 |
| gbc2 | 0 | 3.75E−05 | 6.09E−07 | 0 | 0.010782 | 0 | 3.45E−05 | 0 | 0.001247 | 0.000364 |
| gbc3 | 1.51E−05 | 0.000298 | 0 | 0 | 0 | 0.007034 | 0 | 0 | 0.041335 | 0 |
| gbc4 | 0.000906 | 0.000134 | 0 | 0 | 3.45E−05 | 0.000106 | 0 | 0 | 0.02302 | 0 |
| gbc5 | 0.000276 | 0.009984 | 0 | 5.43E−05 | 0.01135 | 0 | 0 | 6.04E−05 | 0 | 0 |
| gbc6 | 0 | 0 | 0.003086 | 0.003136 | 0 | 0 | 5.73E−05 | 0 | 0.011401 | 0 |
| gbc7 | 0 | 0 | 0 | 0.005056 | 0 | 0.014684 | 2.90E−05 | 0 | 0.005417 | 1.21E−05 |
| gbc8 | 0 | 0 | 5.90E−07 | 0.000981 | 0.002613 | 0 | 0.003261 | 0 | 0 | 3.01E−06 |
| gbc9 | 0 | 0 | 0 | 0 | 0 | 0 | 0.001986 | 0.001304 | 5.47E−05 | 0 |
| gbc10 | 0 | 0 | 0 | 0 | 0 | 0.000682 | 4.98E−05 | 0 | 6.06E−05 | 0 |
| gbc11 | 0 | 0.001592 | 0 | 0.002568 | 0 | 2.46E−05 | 0 | 0 | 1.71E−05 | 0 |
| gbc12 | 0 | 8.12E−06 | 0.002359 | 0.001767 | 0 | 0 | 1.85E−07 | 0 | 0.000331 | 0 |
| gbc13 | 0.000369 | 0 | 0 | 0.000322 | 0.003357 | 0 | 0 | 0 | 0 | 1.69E−05 |
| gbc14 | 0 | 8.31E−07 | 0.002032 | 0 | 0 | 0 | 0 | 0 | 0.000102 | 1.21E−05 |
| gbc15 | 0 | 0 | 0.001315 | 5.23E−05 | 0 | 0 | 0 | 0 | 0.008454 | 0 |
| gbc16 | 0 | 3.75E−05 | 6.09E−07 | 0 | 0.010782 | 0 | 3.45E−05 | 0 | 0.001247 | 0.000364 |
| gbc17 | 1.51E−05 | 0.000298 | 0 | 0 | 0 | 0.007034 | 0 | 0 | 0.041335 | 0 |
| gbc18 | 0.000906 | 0.000134 | 0 | 0 | 3.45E−05 | 0.000106 | 0 | 0 | 0.02302 | 0 |
| gbc19 | 0.000276 | 0.009984 | 0 | 5.43E−05 | 0.01135 | 0 | 0 | 6.04E−05 | 0 | 0 |
| gbc20 | 0 | 0 | 0.003086 | 0.003136 | 0 | 0 | 5.73E−05 | 0 | 0.011401 | 0 |
| gbc21 | 0 | 0 | 0 | 0.005056 | 0 | 0.014684 | 2.90E−05 | 0 | 0.005417 | 1.21E−05 |
| gbc22 | 0 | 0 | 5.90E−07 | 0.000981 | 0.002613 | 0 | 0.003261 | 0 | 0 | 3.01E−06 |
| gbc23 | 0 | 0 | 0 | 0 | 0 | 0 | 0.001986 | 0.001304 | 5.47E−05 | 0 |
| gbc24 | 0 | 0 | 0 | 0 | 0 | 0.000682 | 4.98E−05 | 0 | 6.06E−05 | 0 |
| gbc25 | 0 | 0.001592 | 0 | 0.002568 | 0 | 2.46E−05 | 0 | 0 | 1.71E−05 | 0 |
| gbc26 | 0 | 8.12E−06 | 0.002359 | 0.001767 | 0 | 0 | 1.85E−07 | 0 | 0.000331 | 0 |
| gbc27 | 0.000369 | 0 | 0 | 0.000322 | 0.003357 | 0 | 0 | 0 | 0 | 1.69E−05 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | BIRC3 | BLZF1 | BSCL2 | BSDC1 | BTG3 | C10orf88 | C12orf29 | C17orf58 | C1orf198 | C21orf91 |
|---|---|---|---|---|---|---|---|---|---|---|
| rf0 | 8.38E−19 | 0.003774 | 0 | 0 | 0.000285 | 0.007027 | 0.00065 | 0.002236 | 0.001737 | 0 |
| rf1 | 0.004683 | 3.30E−17 | 0.001127 | 0.001389 | 0.002624 | 0.002731 | 0.001457 | 0.004245 | 0.00287 | 0 |
| rf2 | 0 | 5.49E−19 | 0 | 0.000398 | 0.000466 | 0.00314 | 0.000358 | 0.000662 | 0 | 0 |
| rf3 | 0.003467 | 0.001153 | 0.000321 | 0 | 0.002665 | 0.009144 | 0.000647 | 0.00243 | 0.005147 | 0 |
| rf4 | 0.000786 | 0.001903 | 0 | 0.000346 | 0.003304 | 0 | 0 | 0.000764 | 2.52E−19 | 0.003302 |
| rf5 | 0.000298 | 0.001023 | 0 | 0.005198 | 0.004455 | 0.000691 | 0.002708 | 0.001738 | 0.000384 | 0.000363 |
| rf6 | 7.85E−05 | 0.000697 | 0.002088 | 0.001504 | 0.006995 | 0.010756 | 0.007465 | 0.001384 | 0 | 1.58E−18 |
| rf7 | 0.001285 | 0.00163 | 0.000393 | 0 | 0.001356 | 0.010621 | 0.001053 | 0.001201 | 0.002617 | 0 |
| rf8 | 0.003644 | 0.002841 | 0.003684 | 0 | 0.001115 | 0.005853 | 0.002438 | 0.00079 | 0.014654 | 0.001688 |
| rf9 | 1.92E−18 | 0 | 1.10E−18 | 8.35E−18 | 0.001776 | 0.000357 | 0 | 0.002834 | 0.002053 | 0.001136 |
| rf10 | 0 | 0.001413 | 0.000396 | 7.73E−05 | 0.001806 | 0.005351 | 0.001736 | 0 | 0 | 0.003411 |
| rf11 | 0.002646 | 0 | 3.83E−18 | 2.71E−05 | 3.98E−18 | 0.00701 | 0 | 2.05E−05 | 0 | 0.006738 |
| rf12 | 0.006103 | 0.00743 | 0.000725 | 0.001174 | 0.001857 | 0 | 7.91E−05 | 0.004904 | 0.002963 | 0.003532 |
| rf13 | 1.52E−18 | 0.004978 | 0 | 0 | 0.004992 | 0.000426 | 0 | 0.004224 | 0.005346 | 2.42E−18 |
| rf14 | 8.38E−19 | 0.003774 | 0 | 0 | 0.000285 | 0.007027 | 0.00065 | 0.002236 | 0.001737 | 0 |
| rf15 | 0.004683 | 3.30E−17 | 0.001127 | 0.001389 | 0.002624 | 0.002731 | 0.001457 | 0.004245 | 0.00287 | 0 |
| rf16 | 0 | 5.49E−19 | 0 | 0.000398 | 0.000466 | 0.00314 | 0.000358 | 0.000662 | 0 | 0 |
| rf17 | 0.003467 | 0.001153 | 0.000321 | 0 | 0.002665 | 0.009144 | 0.000647 | 0.00243 | 0.005147 | 0 |
| rf18 | 0.000786 | 0.001903 | 0 | 0.000346 | 0.003304 | 0 | 0 | 0.000764 | 2.52E−19 | 0.003302 |
| rf19 | 0.000298 | 0.001023 | 0 | 0.005198 | 0.004455 | 0.000691 | 0.002708 | 0.001738 | 0.000384 | 0.000363 |
| rf20 | 7.85E−05 | 0.000697 | 0.002088 | 0.001504 | 0.006995 | 0.010756 | 0.007465 | 0.001384 | 0 | 1.58E−18 |
| rf21 | 0.001285 | 0.00163 | 0.000393 | 0 | 0.001356 | 0.010621 | 0.001053 | 0.001201 | 0.002617 | 0 |
| rf22 | 0.003644 | 0.002841 | 0.003684 | 0 | 0.001115 | 0.005853 | 0.002438 | 0.00079 | 0.014654 | 0.001688 |
| rf23 | 1.92E−18 | 0 | 1.10E−18 | 8.35E−18 | 0.001776 | 0.000357 | 0 | 0.002834 | 0.002053 | 0.001136 |
| rf24 | 0 | 0.001413 | 0.000396 | 7.73E−05 | 0.001806 | 0.005351 | 0.001736 | 0 | 0 | 0.003411 |
| rf25 | 0.002646 | 0 | 3.83E−18 | 2.71E−05 | 3.98E−18 | 0.00701 | 0 | 2.05E−05 | 0 | 0.006738 |
| rf26 | 0.006103 | 0.00743 | 0.000725 | 0.001174 | 0.001857 | 0 | 7.91E−05 | 0.004904 | 0.002963 | 0.003532 |
| rf27 | 1.52E−18 | 0.004978 | 0 | 0 | 0.004992 | 0.000426 | 0 | 0.004224 | 0.005346 | 2.42E−18 |
| extraTrees0 | 0.002349 | 0.000456 | 0.001723 | 0.000546 | 0.00322 | 0.009562 | 0.000546 | 0.003072 | 0.000815 | 0.000906 |
| extraTrees1 | 0.000248 | 0.000727 | 0.001542 | 6.78E−18 | 0.002991 | 0.005877 | 0.001922 | 0.002361 | 0.000781 | 0.002796 |
| extraTrees2 | 0.001284 | 0.001337 | 0.002398 | 0.000574 | 0.001106 | 0.001606 | 0.000249 | 0.002526 | 0.00133 | 0.000718 |
| extraTrees3 | 0.000519 | 0.001273 | 0.002309 | 0.001015 | 0.002749 | 0.003463 | 0.002378 | 0.000819 | 0.002844 | 0 |
| extraTrees4 | 0.001138 | 0.00048 | 0.001834 | 0.000988 | 0.002531 | 0.009685 | 0.002666 | 0.003772 | 0.001459 | 0.001461 |
| extraTrees5 | 0.001059 | 0.000741 | 0.00169 | 0.000917 | 0.001031 | 0.00547 | 6.87E−18 | 0.00086 | 0.000928 | 0.000968 |
| extraTrees6 | 0.001346 | 0.001222 | 0.002882 | 0.004637 | 0.003673 | 0.008885 | 0.000699 | 0.003201 | 0.002041 | 0 |
| extraTrees7 | 0.000819 | 0.000751 | 5.26E−05 | 0.000273 | 0.002777 | 0.001629 | 0.000971 | 0.00244 | 0.003092 | 0.000796 |
| extraTrees8 | 0.000542 | 0.001089 | 0.001691 | 0.001272 | 0.000896 | 0.010173 | 0.001216 | 0.001374 | 0.001032 | 0.001766 |
| extraTrees9 | 0.000888 | 0.001023 | 0.001296 | 0.000846 | 0.001069 | 0.005026 | 0.002105 | 0.003034 | 0.004069 | 0.001408 |
| extraTrees10 | 0.001842 | 0.00272 | 0.000495 | 0.001195 | 0.002244 | 0.003229 | 0.00118 | 0.004561 | 0.001638 | 0.000325 |
| extraTrees11 | 0.001803 | 0.001143 | 0.000819 | 0.000554 | 0.004614 | 0.008862 | 0.000431 | 0.0019 | 0.002621 | 0 |
| extraTrees12 | 0.001395 | 0.001593 | 0.000297 | 0.000907 | 0.005479 | 0.003397 | 0.001977 | 0.003938 | 0.002305 | 0.000903 |
| extraTrees13 | 0.001493 | 0.001567 | 0.0013 | 0.00112 | 0.001248 | 0.004457 | 0.001008 | 0.001155 | 0.001986 | 0.0007 |
| extraTrees14 | 0.002349 | 0.000456 | 0.001723 | 0.000546 | 0.00322 | 0.009562 | 0.000546 | 0.003072 | 0.000815 | 0.000906 |
| extraTrees15 | 0.000248 | 0.000727 | 0.001542 | 6.78E−18 | 0.002991 | 0.005877 | 0.001922 | 0.002361 | 0.000781 | 0.002796 |
| extraTrees16 | 0.001284 | 0.001337 | 0.002398 | 0.000574 | 0.001106 | 0.001606 | 0.000249 | 0.002526 | 0.00133 | 0.000718 |
| extraTrees17 | 0.000519 | 0.001273 | 0.002309 | 0.001015 | 0.002749 | 0.003463 | 0.002378 | 0.000819 | 0.002844 | 0 |
| extraTrees18 | 0.001138 | 0.00048 | 0.001834 | 0.000988 | 0.002531 | 0.009685 | 0.002666 | 0.003772 | 0.001459 | 0.001461 |
| extraTrees19 | 0.001059 | 0.000741 | 0.00169 | 0.000917 | 0.001031 | 0.00547 | 6.87E−18 | 0.00086 | 0.000928 | 0.000968 |
| extraTrees20 | 0.001346 | 0.001222 | 0.002882 | 0.004637 | 0.003673 | 0.008885 | 0.000699 | 0.003201 | 0.002041 | 0 |
| extraTrees21 | 0.000819 | 0.000751 | 5.26E−05 | 0.000273 | 0.002777 | 0.001629 | 0.000971 | 0.00244 | 0.003092 | 0.000796 |
| extraTrees22 | 0.000542 | 0.001089 | 0.001691 | 0.001272 | 0.000896 | 0.010173 | 0.001216 | 0.001374 | 0.001032 | 0.001766 |
| extraTrees23 | 0.000888 | 0.001023 | 0.001296 | 0.000846 | 0.001069 | 0.005026 | 0.002105 | 0.003034 | 0.004069 | 0.001408 |
| extraTrees24 | 0.001842 | 0.00272 | 0.000495 | 0.001195 | 0.002244 | 0.003229 | 0.00118 | 0.004561 | 0.001638 | 0.000325 |
| extraTrees25 | 0.001803 | 0.001143 | 0.000819 | 0.000554 | 0.004614 | 0.008862 | 0.000431 | 0.0019 | 0.002621 | 0 |
| extraTrees26 | 0.001395 | 0.001593 | 0.000297 | 0.000907 | 0.005479 | 0.003397 | 0.001977 | 0.003938 | 0.002305 | 0.000903 |
| extraTrees27 | 0.001493 | 0.001567 | 0.0013 | 0.00112 | 0.001248 | 0.004457 | 0.001008 | 0.001155 | 0.001986 | 0.0007 |
| gbc0 | 0.000388 | 3.11E−05 | 0.009372 | 0 | 0.000133 | 0.00162 | 0 | 0.019901 | 0 | 0 |
| gbc1 | 0 | 0 | 0 | 0 | 0.005096 | 0.007592 | 0 | 0.001298 | 7.27E−05 | 0 |
| gbc2 | 0.005832 | 0 | 0 | 0 | 0.000661 | 0.005038 | 0 | 0 | 0.000122 | 0 |
| gbc3 | 0 | 0 | 0 | 2.12E−06 | 0.006656 | 0.000252 | 0 | 0.017846 | 0.009215 | 0.000101 |
| gbc4 | 0.001721 | 0.00576 | 3.63E−06 | 0 | 0.003881 | 0.024404 | 0 | 0 | 0.000361 | 0 |
| gbc5 | 0 | 0 | 0 | 8.13E−05 | 0.008118 | 0.014294 | 0 | 0.000525 | 0.000966 | 0 |
| gbc6 | 9.28E−06 | 0 | 0 | 0 | 0.000838 | 0.00187 | 0.000686 | 0.003211 | 0 | 0.000105 |
| gbc7 | 0 | 0.000102 | 0 | 0 | 0.000293 | 0.022176 | 0 | 4.72E−05 | 0 | 6.03E−06 |
| gbc8 | 0 | 0 | 0.000137 | 5.17E−05 | 0 | 0 | 0 | 0.001275 | 6.30E−05 | 1.22E−05 |
| gbc9 | 6.24E−05 | 0.010396 | 0 | 0.005658 | 2.65E−06 | 0.005609 | 0 | 6.03E−05 | 5.43E−05 | 0.002007 |
| gbc10 | 0 | 0 | 0 | 0 | 0.002302 | 0 | 0 | 0.001242 | 0.00026 | 0.002351 |
| gbc11 | 4.77E−05 | 0 | 4.22E−07 | 0.000169 | 0 | 0.023433 | 0 | 0.000162 | 0.000196 | 0 |
| gbc12 | 0 | 0 | 2.38E−06 | 0.000656 | 0.010413 | 0.003949 | 0.021297 | 0 | 0.000124 | 8.00E−06 |
| gbc13 | 0 | 2.56E−06 | 0 | 0 | 0.000147 | 0 | 0 | 0 | 0.003035 | 0.000748 |
| gbc14 | 0.000388 | 3.11E−05 | 0.009372 | 0 | 0.000133 | 0.00162 | 0 | 0.019901 | 0 | 0 |
| gbc15 | 0 | 0 | 0 | 0 | 0.005096 | 0.007592 | 0 | 0.001298 | 7.27E−05 | 0 |
| gbc16 | 0.005832 | 0 | 0 | 0 | 0.000661 | 0.005038 | 0 | 0 | 0.000122 | 0 |
| gbc17 | 0 | 0 | 0 | 2.12E−06 | 0.006656 | 0.000252 | 0 | 0.017846 | 0.009215 | 0.000101 |
| gbc18 | 0.001721 | 0.00576 | 3.63E−06 | 0 | 0.003881 | 0.024404 | 0 | 0 | 0.000361 | 0 |
| gbc19 | 0 | 0 | 0 | 8.13E−05 | 0.008118 | 0.014294 | 0 | 0.000525 | 0.000966 | 0 |
| gbc20 | 9.28E−06 | 0 | 0 | 0 | 0.000838 | 0.00187 | 0.000686 | 0.003211 | 0 | 0.000105 |
| gbc21 | 0 | 0.000102 | 0 | 0 | 0.000293 | 0.022176 | 0 | 4.72E−05 | 0 | 6.03E−06 |
| gbc22 | 0 | 0 | 0.000137 | 5.17E−05 | 0 | 0 | 0 | 0.001275 | 6.30E−05 | 1.22E−05 |
| gbc23 | 6.24E−05 | 0.010396 | 0 | 0.005658 | 2.65E−06 | 0.005609 | 0 | 6.03E−05 | 5.43E−05 | 0.002007 |
| gbc24 | 0 | 0 | 0 | 0 | 0.002302 | 0 | 0 | 0.001242 | 0.00026 | 0.002351 |
| gbc25 | 4.77E−05 | 0 | 4.22E−07 | 0.000169 | 0 | 0.023433 | 0 | 0.000162 | 0.000196 | 0 |
| gbc26 | 0 | 0 | 2.38E−06 | 0.000656 | 0.010413 | 0.003949 | 0.021297 | 0 | 0.000124 | 8.00E−06 |
| gbc27 | 0 | 2.56E−06 | 0 | 0 | 0.000147 | 0 | 0 | 0 | 0.003035 | 0.000748 |
| ada0 | 0 | 0 | 0 | 0 | 0.02 | 0 | 0 | 0 | 0 | 0 |
| ada1 | 0 | 0 | 0 | 0 | 0.02 | 0 | 0 | 0 | 0 | 0 |
| ada2 | 0 | 0 | 0 | 0 | 0.02 | 0 | 0 | 0 | 0 | 0 |
| ada3 | 0 | 0 | 0 | 0 | 0.02 | 0 | 0 | 0 | 0 | 0 |
| ada4 | 0 | 0 | 0 | 0 | 0.02 | 0 | 0 | 0 | 0 | 0 |
| ada5 | 0 | 0 | 0 | 0 | 0.02 | 0 | 0 | 0 | 0 | 0 |
| ada6 | 0 | 0 | 0 | 0 | 0.02 | 0 | 0 | 0 | 0 | 0 |
| ada7 | 0 | 0 | 0 | 0 | 0.02 | 0 | 0 | 0 | 0 | 0 |
| ada8 | 0 | 0 | 0 | 0 | 0.02 | 0 | 0 | 0 | 0 | 0 |
| ada9 | 0 | 0 | 0 | 0 | 0.02 | 0 | 0 | 0 | 0 | 0 |
| ada10 | 0 | 0 | 0 | 0 | 0.02 | 0 | 0 | 0 | 0 | 0 |
| ada11 | 0 | 0 | 0 | 0 | 0.02 | 0 | 0 | 0 | 0 | 0 |
| ada12 | 0 | 0 | 0 | 0 | 0.02 | 0 | 0 | 0 | 0 | 0 |
| ada13 | 0 | 0 | 0 | 0 | 0.02 | 0 | 0 | 0 | 0 | 0 |
| ada14 | 0 | 0 | 0 | 0 | 0.02 | 0 | 0 | 0 | 0 | 0 |
| ada15 | 0 | 0 | 0 | 0 | 0.02 | 0 | 0 | 0 | 0 | 0 |
| ada16 | 0 | 0 | 0 | 0 | 0.02 | 0 | 0 | 0 | 0 | 0 |
| ada17 | 0 | 0 | 0 | 0 | 0.02 | 0 | 0 | 0 | 0 | 0 |
| ada18 | 0 | 0 | 0 | 0 | 0.02 | 0 | 0 | 0 | 0 | 0 |
| ada19 | 0 | 0 | 0 | 0 | 0.02 | 0 | 0 | 0 | 0 | 0 |
| ada20 | 0 | 0 | 0 | 0 | 0.02 | 0 | 0 | 0 | 0 | 0 |
| ada21 | 0 | 0 | 0 | 0 | 0.02 | 0 | 0 | 0 | 0 | 0 |
| ada22 | 0 | 0 | 0 | 0 | 0.02 | 0 | 0 | 0 | 0 | 0 |
| ada23 | 0 | 0 | 0 | 0 | 0.02 | 0 | 0 | 0 | 0 | 0 |
| ada24 | 0 | 0 | 0 | 0 | 0.02 | 0 | 0 | 0 | 0 | 0 |
| ada25 | 0 | 0 | 0 | 0 | 0.02 | 0 | 0 | 0 | 0 | 0 |
| ada26 | 0 | 0 | 0 | 0 | 0.02 | 0 | 0 | 0 | 0 | 0 |
| ada27 | 0 | 0 | 0 | 0 | 0.02 | 0 | 0 | 0 | 0 | 0 |

| Gene | C2orf68 | C3orf38 | C5orf51 | C6orf106 | CALCOCO1 | CAPN1 | CAPN2 | CAVIN4 | CCDC25 | CCDC82 |
|---|---|---|---|---|---|---|---|---|---|---|
| rf0 | 0 | 0.004225 | 0.000504 | 0.001184 | 0.000906 | 0.002741 | 0.004949 | 0.000367 | 0.001614 | 0 |
| rf1 | 0 | 0.001592 | 0.006145 | 0.003128 | 0.000776 | 0 | 0.00042 | 0.003875 | 0.004014 | 8.69E−05 |
| rf2 | 0.006317 | 0.006137 | 0.005303 | 4.27E−18 | 0.000772 | 0.007325 | 0.003998 | 0.002031 | 0 | 0.000645 |
| rf3 | 0 | 0.001399 | 0.00725 | 0.000831 | 0.004939 | 0.000395 | 0.003579 | 0.001531 | 0 | 0 |
| rf4 | 2.60E−05 | 0.004927 | 0.006081 | 0 | 0 | 0.006681 | 0.006418 | 0.003513 | 0.00365 | 0 |
| rf5 | 0.00154 | 0 | 0.004227 | 0.003015 | 0 | 0 | 0.003078 | 0 | 0.003647 | 3.01E−17 |
| rf6 | 0 | 1.21E−17 | 0.012607 | 7.75E−05 | 0.004252 | 0.001529 | 0.01235 | 0 | 0.001352 | 5.87E−18 |
| rf7 | 0.002169 | 0.001133 | 0.013625 | 0.000615 | 0 | 0 | 0.002949 | 0.003049 | 0.000785 | 0.00075 |
| rf8 | 0.000769 | 0 | 0.009813 | 0.002332 | 0.004392 | 0.000951 | 0.001726 | 0.000417 | 0 | 8.05E−18 |
| rf9 | 4.85E−19 | 0 | 0.000446 | 0 | 0.003248 | 0.001966 | 0.002711 | 4.08E−18 | 0.003058 | 4.70E−19 |
| rf10 | 0.000576 | 0 | 0.001453 | 0.000761 | 0 | 0 | 0.002701 | 0 | 0.01031 | 0.001228 |
| rf11 | 0.00417 | 0 | 0.004191 | 0.000329 | 0.001574 | 0 | 0.008277 | 0 | 0.000732 | 0 |
| rf12 | 0.000666 | 0 | 0.001951 | 0.001956 | 0.001283 | 0.00025 | 0.007252 | 0.001165 | 0 | 0.000697 |
| rf13 | 0.001167 | 0.00601 | 0.003537 | 0.003484 | 0 | 0 | 0 | 0 | 0.000363 | 0.002591 |
| rf14 | 0 | 0.004225 | 0.000504 | 0.001184 | 0.000906 | 0.002741 | 0.004949 | 0.000367 | 0.001614 | 0 |
| rf15 | 0 | 0.001592 | 0.006145 | 0.003128 | 0.000776 | 0 | 0.00042 | 0.003875 | 0.004014 | 8.69E−05 |
| rf16 | 0.006317 | 0.006137 | 0.005303 | 4.27E−18 | 0.000772 | 0.007325 | 0.003998 | 0.002031 | 0 | 0.000645 |
| rf17 | 0 | 0.001399 | 0.00725 | 0.000831 | 0.004939 | 0.000395 | 0.003579 | 0.001531 | 0 | 0 |
| rf18 | 2.60E−05 | 0.004927 | 0.006081 | 0 | 0 | 0.006681 | 0.006418 | 0.003513 | 0.00365 | 0 |
| rf19 | 0.00154 | 0 | 0.004227 | 0.003015 | 0 | 0 | 0.003078 | 0 | 0.003647 | 3.01E−17 |
| rf20 | 0 | 1.21E−17 | 0.012607 | 7.75E−05 | 0.004252 | 0.001529 | 0.01235 | 0 | 0.001352 | 5.87E−18 |
| rf21 | 0.002169 | 0.001133 | 0.013625 | 0.000615 | 0 | 0 | 0.002949 | 0.003049 | 0.000785 | 0.00075 |
| rf22 | 0.000769 | 0 | 0.009813 | 0.002332 | 0.004392 | 0.000951 | 0.001726 | 0.000417 | 0 | 8.05E−18 |
| rf23 | 4.85E−19 | 0 | 0.000446 | 0 | 0.003248 | 0.001966 | 0.002711 | 4.08E−18 | 0.003058 | 4.70E−19 |
| rf24 | 0.000576 | 0 | 0.001453 | 0.000761 | 0 | 0 | 0.002701 | 0 | 0.01031 | 0.001228 |
| rf25 | 0.00417 | 0 | 0.004191 | 0.000329 | 0.001574 | 0 | 0.008277 | 0 | 0.000732 | 0 |
| rf26 | 0.000666 | 0 | 0.001951 | 0.001956 | 0.001283 | 0.00025 | 0.007252 | 0.001165 | 0 | 0.000697 |
| rf27 | 0.001167 | 0.00601 | 0.003537 | 0.003484 | 0 | 0 | 0 | 0 | 0.000363 | 0.002591 |
| extraTrees0 | 0 | 0.003332 | 0.006572 | 0.00135 | 0.001146 | 0.001062 | 0.006382 | 0.000402 | 0.001906 | 0.000238 |
| extraTrees1 | 0.001521 | 0.001491 | 0.005798 | 0.002967 | 0.002146 | 0.003825 | 0.006001 | 0.000876 | 0.002004 | 0.001234 |
| extraTrees2 | 0.005881 | 0.001927 | 0.003807 | 0.003263 | 0.002046 | 0.000929 | 0.002047 | 0.001084 | 0.00383 | 5.27E−17 |
| extraTrees3 | 0.00201 | 0.002401 | 0.004976 | 0.002465 | 7.01E−19 | 0.001484 | 0.003908 | 0.001217 | 0.004227 | 1.26E−18 |
| extraTrees4 | 0.00064 | 0.000238 | 0.007423 | 0.002309 | 0.001041 | 0.001369 | 0.005862 | 0.00133 | 0.001755 | 0.002898 |
| extraTrees5 | 0.001173 | 0.001736 | 0.004831 | 0.001796 | 1.90E−18 | 2.73E−19 | 0.001926 | 0.000726 | 0.002825 | 0.000775 |
| extraTrees6 | 0.002749 | 1.43E−18 | 0.001607 | 0.000343 | 0 | 0.000978 | 0.011514 | 0.000778 | 0.002848 | 0.000361 |
| extraTrees7 | 0 | 2.12E−18 | 0.005118 | 0.001045 | 0.001054 | 0.002013 | 0.006392 | 0.002233 | 0.002936 | 0.000339 |
| extraTrees8 | 0.003678 | 0.002778 | 0.00661 | 0.003604 | 0.001192 | 0.00255 | 0.008627 | 0.000238 | 0.005158 | 0.001646 |
| extraTrees9 | 0.000238 | 0.001504 | 0.010762 | 0 | 0.000378 | 0.000467 | 0.005254 | 0.001294 | 0.004545 | 0.000298 |
| extraTrees10 | 0.003018 | 0.001641 | 0.003415 | 0.002762 | 7.17E−19 | 0.002359 | 0.001624 | 0.002988 | 0.002515 | 0.000511 |
| extraTrees11 | 5.20E−05 | 0.000497 | 0.012051 | 0.00166 | 0.000709 | 0.001674 | 0.005921 | 0.002012 | 0.002002 | 0.001041 |
| extraTrees12 | 0.00065 | 0.001743 | 0.004303 | 0.000563 | 5.26E−19 | 0.002883 | 0.004525 | 0.001223 | 0.003149 | 0.000222 |
| extraTrees13 | 0.002429 | 0.000707 | 0.001964 | 0.003182 | 0.001739 | 0.000238 | 0.004624 | 0.001203 | 0.002667 | 0 |
| extraTrees14 | 0 | 0.003332 | 0.006572 | 0.00135 | 0.001146 | 0.001062 | 0.006382 | 0.000402 | 0.001906 | 0.000238 |
| extraTrees15 | 0.001521 | 0.001491 | 0.005798 | 0.002967 | 0.002146 | 0.003825 | 0.006001 | 0.000876 | 0.002004 | 0.001234 |
| extraTrees16 | 0.005881 | 0.001927 | 0.003807 | 0.003263 | 0.002046 | 0.000929 | 0.002047 | 0.001084 | 0.00383 | 5.27E−17 |
| extraTrees17 | 0.00201 | 0.002401 | 0.004976 | 0.002465 | 7.01E−19 | 0.001484 | 0.003908 | 0.001217 | 0.004227 | 1.26E−18 |
| extraTrees18 | 0.00064 | 0.000238 | 0.007423 | 0.002309 | 0.001041 | 0.001369 | 0.005862 | 0.00133 | 0.001755 | 0.002898 |
| extraTrees19 | 0.001173 | 0.001736 | 0.004831 | 0.001796 | 1.90E−18 | 2.73E−19 | 0.001926 | 0.000726 | 0.002825 | 0.000775 |
| extraTrees20 | 0.002749 | 1.43E−18 | 0.001607 | 0.000343 | 0 | 0.000978 | 0.011514 | 0.000778 | 0.002848 | 0.000361 |
| extraTrees21 | 0 | 2.12E−18 | 0.005118 | 0.001045 | 0.001054 | 0.002013 | 0.006392 | 0.002233 | 0.002936 | 0.000339 |
| extraTrees22 | 0.003678 | 0.002778 | 0.00661 | 0.003604 | 0.001192 | 0.00255 | 0.008627 | 0.000238 | 0.005158 | 0.001646 |
| extraTrees23 | 0.000238 | 0.001504 | 0.010762 | 0 | 0.000378 | 0.000467 | 0.005254 | 0.001294 | 0.004545 | 0.000298 |
| extraTrees24 | 0.003018 | 0.001641 | 0.003415 | 0.002762 | 7.17E−19 | 0.002359 | 0.001624 | 0.002988 | 0.002515 | 0.000511 |
| extraTrees25 | 5.20E−05 | 0.000497 | 0.012051 | 0.00166 | 0.000709 | 0.001674 | 0.005921 | 0.002012 | 0.002002 | 0.001041 |
| extraTrees26 | 0.00065 | 0.001743 | 0.004303 | 0.000563 | 5.26E−19 | 0.002883 | 0.004525 | 0.001223 | 0.003149 | 0.000222 |
| extraTrees27 | 0.002429 | 0.000707 | 0.001964 | 0.003182 | 0.001739 | 0.000238 | 0.004624 | 0.001203 | 0.002667 | 0 |
| gbc0 | 7.34E−05 | 1.30E−05 | 0.009537 | 0.001057 | 0 | 0 | 0.000855 | 0 | 0.000124 | 0 |
| gbc1 | 2.10E−05 | 0.000102 | 0.000113 | 4.07E−06 | 0.000623 | 0.00993 | 0.001792 | 1.19E−05 | 0 | 0 |
| gbc2 | 0 | 0 | 0.003632 | 0 | 0.000418 | 0 | 9.88E−05 | 0.000488 | 0.012871 | 0.000101 |
| gbc3 | 0 | 0 | 0 | 0.008604 | 0 | 3.49E−05 | 7.07E−05 | 0 | 0.020476 | 0 |
| gbc4 | 0.00365 | 0.000351 | 0.014951 | 0 | 0.000142 | 0 | 0 | 8.08E−07 | 0.007743 | 0 |
| gbc5 | 0 | 2.92E−05 | 0.001136 | 8.82E−05 | 0 | 8.53E−05 | 0 | 0 | 0.001999 | 0.000254 |
| gbc6 | 0.007137 | 0 | 0.012802 | 6.10E−05 | 0 | 0.00061 | 0.001628 | 0 | 4.05E−05 | 0 |
| gbc7 | 0 | 0 | 0.004684 | 0.000378 | 0.000348 | 0 | 0.003354 | 0.008311 | 0 | 0 |
| gbc8 | 0 | 0.001806 | 0.020158 | 0.00028 | 0.000192 | 7.67E−05 | 0.002471 | 0 | 1.50E−06 | 0.015271 |
| gbc9 | 0.000181 | 0.020106 | 0.001528 | 0.004214 | 0 | 0 | 0 | 0.00158 | 0 | 0.008062 |
| gbc10 | 0.00016 | 0 | 0.011589 | 0.000352 | 0 | 0 | 0.016605 | 0 | 0 | 0 |
| gbc11 | 0.000905 | 0.000274 | 0.004318 | 0.023708 | 0 | 0 | 1.49E−05 | 0.000876 | 0.00433 | 0 |
| gbc12 | 0.001081 | 0 | 0.00089 | 7.64E−05 | 0.000319 | 4.88E−06 | 0.016654 | 0 | 0 | 0.001888 |
| gbc13 | 0.000279 | 0.003884 | 0.004913 | 0.000268 | 0 | 0 | 0.000874 | 0 | 0 | 0 |
| gbc14 | 7.34E−05 | 1.30E−05 | 0.009537 | 0.001057 | 0 | 0 | 0.000855 | 0 | 0.000124 | 0 |
| gbc15 | 2.10E−05 | 0.000102 | 0.000113 | 4.07E−06 | 0.000623 | 0.00993 | 0.001792 | 1.19E−05 | 0 | 0 |
| gbc16 | 0 | 0 | 0.003632 | 0 | 0.000418 | 0 | 9.88E−05 | 0.000488 | 0.012871 | 0.000101 |
| gbc17 | 0 | 0 | 0 | 0.008604 | 0 | 3.49E−05 | 7.07E−05 | 0 | 0.020476 | 0 |
| gbc18 | 0.00365 | 0.000351 | 0.014951 | 0 | 0.000142 | 0 | 0 | 8.08E−07 | 0.007743 | 0 |
| gbc19 | 0 | 2.92E−05 | 0.001136 | 8.82E−05 | 0 | 8.53E−05 | 0 | 0 | 0.001999 | 0.000254 |
| gbc20 | 0.007137 | 0 | 0.012802 | 6.10E−05 | 0 | 0.00061 | 0.001628 | 0 | 4.05E−05 | 0 |
| gbc21 | 0 | 0 | 0.004684 | 0.000378 | 0.000348 | 0 | 0.003354 | 0.008311 | 0 | 0 |
| gbc22 | 0 | 0.001806 | 0.020158 | 0.00028 | 0.000192 | 7.67E−05 | 0.002471 | 0 | 1.50E−06 | 0.015271 |
| gbc23 | 0.000181 | 0.020106 | 0.001528 | 0.004214 | 0 | 0 | 0 | 0.00158 | 0 | 0.008062 |
| gbc24 | 0.00016 | 0 | 0.011589 | 0.000352 | 0 | 0 | 0.016605 | 0 | 0 | 0 |
| gbc25 | 0.000905 | 0.000274 | 0.004318 | 0.023708 | 0 | 0 | 1.49E−05 | 0.000876 | 0.00433 | 0 |
| gbc26 | 0.001081 | 0 | 0.00089 | 7.64E−05 | 0.000319 | 4.88E−06 | 0.016654 | 0 | 0 | 0.001888 |
| gbc27 | 0.000279 | 0.003884 | 0.004913 | 0.000268 | 0 | 0 | 0.000874 | 0 | 0 | 0 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | CCDC88C | CCDC9B | CCL18 | CD69 | CDC37 | CDKN2AIP | CEP120 | CEP170 | CHODL-AS1 | CKAP2L |
|---|---|---|---|---|---|---|---|---|---|---|
| rf0 | 0.001071 | 0.001128 | 0 | 0.002477 | 0 | 0 | 0.006912 | 0.000403 | 0.004339 | 0 |
| rf1 | 0.001057 | 0.004418 | 0.001343 | 0.003017 | 0.003276 | 0.001146 | 0.006388 | 0.001578 | 0 | 0 |
| rf2 | 0.006124 | 0 | 0.004239 | 1.56E−18 | 0 | 0 | 0.004362 | 0.001083 | 0.001978 | 0 |
| rf3 | 0.003222 | 0.003247 | 0.002761 | 0.00086 | 0.000771 | 0.000412 | 0.000274 | 0.004369 | 0 | 0.005986 |
| rf4 | 0.001329 | 0 | 0 | 0.000357 | 0.001948 | 0.000878 | 0.013242 | 0.005635 | 0.004581 | 0 |
| rf5 | 0.001452 | 0.001536 | 0.003351 | 0.011338 | 0 | 0 | 0.004599 | 0.007927 | 0 | 0 |
| rf6 | 0.00135 | 0.003589 | 0.00511 | 0.000414 | 0.000353 | 0.000588 | 0.004817 | 1.01E−18 | 0 | 0.003077 |
| rf7 | 0 | 0.004598 | 4.73E−18 | 0.001936 | 0 | 0 | 0.004007 | 0.002522 | 0.000581 | 1.18E−17 |
| rf8 | 0 | 0.0071 | 6.19E−18 | 3.61E−18 | 0.000687 | 0.001838 | 0.000459 | 0.002564 | 0 | 0.00024 |
| rf9 | 0.003565 | 6.37E−19 | 5.03E−18 | 0.002815 | 0 | 0 | 0.002855 | 0.000321 | 0.003923 | 0 |
| rf10 | 0.003727 | 0.005917 | 0.007894 | 0.000349 | 0.001454 | 0.004826 | 0.012353 | 0.001406 | 0.000343 | 0 |
| rf11 | 0 | 0 | 0.004181 | 0.010185 | 0 | 0.002943 | 0.011571 | 0 | 0.002639 | 0 |
| rf12 | 0.002471 | 0.001363 | 0.003285 | 0.003984 | 0 | 0.001382 | 0.005341 | 0.004418 | 0 | 0 |
| rf13 | 0.000709 | 0.000632 | 4.07E−05 | 0 | 0.000737 | 0.001814 | 0.008566 | 0.001414 | 0 | 0.000788 |
| rf14 | 0.001071 | 0.001128 | 0 | 0.002477 | 0 | 0 | 0.006912 | 0.000403 | 0.004339 | 0 |
| rf15 | 0.001057 | 0.004418 | 0.001343 | 0.003017 | 0.003276 | 0.001146 | 0.006388 | 0.001578 | 0 | 0 |
| rf16 | 0.006124 | 0 | 0.004239 | 1.56E−18 | 0 | 0 | 0.004362 | 0.001083 | 0.001978 | 0 |
| rf17 | 0.003222 | 0.003247 | 0.002761 | 0.00086 | 0.000771 | 0.000412 | 0.000274 | 0.004369 | 0 | 0.005986 |
| rf18 | 0.001329 | 0 | 0 | 0.000357 | 0.001948 | 0.000878 | 0.013242 | 0.005635 | 0.004581 | 0 |
| rf19 | 0.001452 | 0.001536 | 0.003351 | 0.011338 | 0 | 0 | 0.004599 | 0.007927 | 0 | 0 |
| rf20 | 0.00135 | 0.003589 | 0.00511 | 0.000414 | 0.000353 | 0.000588 | 0.004817 | 1.01E−18 | 0 | 0.003077 |
| rf21 | 0 | 0.004598 | 4.73E−18 | 0.001936 | 0 | 0 | 0.004007 | 0.002522 | 0.000581 | 1.18E−17 |
| rf22 | 0 | 0.0071 | 6.19E−18 | 3.61E−18 | 0.000687 | 0.001838 | 0.000459 | 0.002564 | 0 | 0.00024 |
| rf23 | 0.003565 | 6.37E−19 | 5.03E−18 | 0.002815 | 0 | 0 | 0.002855 | 0.000321 | 0.003923 | 0 |
| rf24 | 0.003727 | 0.005917 | 0.007894 | 0.000349 | 0.001454 | 0.004826 | 0.012353 | 0.001406 | 0.000343 | 0 |
| rf25 | 0 | 0 | 0.004181 | 0.010185 | 0 | 0.002943 | 0.011571 | 0 | 0.002639 | 0 |
| rf26 | 0.002471 | 0.001363 | 0.003285 | 0.003984 | 0 | 0.001382 | 0.005341 | 0.004418 | 0 | 0 |
| rf27 | 0.000709 | 0.000632 | 4.07E−05 | 0 | 0.000737 | 0.001814 | 0.008566 | 0.001414 | 0 | 0.000788 |
| extraTrees0 | 0.008265 | 0.002631 | 0.000515 | 0.002779 | 0.003804 | 0.001571 | 0.006192 | 0.001345 | 0.000618 | 0.001048 |
| extraTrees1 | 0.007034 | 0.000673 | 0.000339 | 0.001882 | 0.00289 | 0.001492 | 0.014737 | 0.00156 | 0.000199 | 0 |
| extraTrees2 | 0.001957 | 0.002376 | 0.00025 | 0.00171 | 0.005726 | 0.000703 | 0.004132 | 0.000581 | 0 | 0.002852 |
| extraTrees3 | 0.005838 | 0.001971 | 0.00302 | 0.001747 | 0.001196 | 0.000868 | 0.008368 | 0.000388 | 0.000491 | 0.002065 |
| extraTrees4 | 0.00508 | 0.001547 | 0.001134 | 0.003999 | 5.26E−19 | 0.00173 | 0.005136 | 0.001041 | 0 | 0.000388 |
| extraTrees5 | 0.003679 | 0.00418 | 0.000557 | 0.000287 | 0.0009 | 0.002909 | 0.001528 | 0.001031 | 0.002509 | 1.49E−18 |
| extraTrees6 | 0.002005 | 0.001904 | 0.002992 | 0.002803 | 0.001561 | 0.001007 | 0.001993 | 0.001866 | 0 | 0.000503 |
| extraTrees7 | 0.001215 | 0.003461 | 0.00324 | 0.001019 | 0.003981 | 0.00037 | 0.005039 | 0.000287 | 0.001331 | 0.001204 |
| extraTrees8 | 0.001617 | 0.00234 | 0.00243 | 0.001621 | 0.001046 | 0.002472 | 0.006866 | 0.002189 | 0.001526 | 0.002107 |
| extraTrees9 | 0.00587 | 0.006434 | 0.00195 | 0.002645 | 0.004286 | 0.00182 | 0.001997 | 0.001407 | 0.000291 | 0.000291 |
| extraTrees10 | 0.002821 | 0.000929 | 0.001748 | 0.002301 | 0.000413 | 0.001229 | 0.005382 | 0.001783 | 0.002475 | 0.001383 |
| extraTrees11 | 0.006095 | 0.003352 | 0.001601 | 0.002011 | 0.002115 | 0.001021 | 0.00357 | 0.001446 | 1.99E−18 | 0.000711 |
| extraTrees12 | 0.001259 | 0.003418 | 0.001994 | 0.002148 | 0.00145 | 0.000443 | 0.00398 | 0.000568 | 0.001303 | 0.001101 |
| extraTrees13 | 0.002009 | 0.001034 | 0.001378 | 0.001981 | 0.00146 | 0.001237 | 0.005339 | 0.003722 | 0 | 0.000257 |
| extraTrees14 | 0.008265 | 0.002631 | 0.000515 | 0.002779 | 0.003804 | 0.001571 | 0.006192 | 0.001345 | 0.000618 | 0.001048 |
| extraTrees15 | 0.007034 | 0.000673 | 0.000339 | 0.001882 | 0.00289 | 0.001492 | 0.014737 | 0.00156 | 0.000199 | 0 |
| extraTrees16 | 0.001957 | 0.002376 | 0.00025 | 0.00171 | 0.005726 | 0.000703 | 0.004132 | 0.000581 | 0 | 0.002852 |
| extraTrees17 | 0.005838 | 0.001971 | 0.00302 | 0.001747 | 0.001196 | 0.000868 | 0.008368 | 0.000388 | 0.000491 | 0.002065 |
| extraTrees18 | 0.00508 | 0.001547 | 0.001134 | 0.003999 | 5.26E−19 | 0.00173 | 0.005136 | 0.001041 | 0 | 0.000388 |
| extraTrees19 | 0.003679 | 0.00418 | 0.000557 | 0.000287 | 0.0009 | 0.002909 | 0.001528 | 0.001031 | 0.002509 | 1.49E−18 |
| extraTrees20 | 0.002005 | 0.001904 | 0.002992 | 0.002803 | 0.001561 | 0.001007 | 0.001993 | 0.001866 | 0 | 0.000503 |
| extraTrees21 | 0.001215 | 0.003461 | 0.00324 | 0.001019 | 0.003981 | 0.00037 | 0.005039 | 0.000287 | 0.001331 | 0.001204 |
| extraTrees22 | 0.001617 | 0.00234 | 0.00243 | 0.001621 | 0.001046 | 0.002472 | 0.006866 | 0.002189 | 0.001526 | 0.002107 |
| extraTrees23 | 0.00587 | 0.006434 | 0.00195 | 0.002645 | 0.004286 | 0.00182 | 0.001997 | 0.001407 | 0.000291 | 0.000291 |
| extraTrees24 | 0.002821 | 0.000929 | 0.001748 | 0.002301 | 0.000413 | 0.001229 | 0.005382 | 0.001783 | 0.002475 | 0.001383 |
| extraTrees25 | 0.006095 | 0.003352 | 0.001601 | 0.002011 | 0.002115 | 0.001021 | 0.00357 | 0.001446 | 1.99E−18 | 0.000711 |
| extraTrees26 | 0.001259 | 0.003418 | 0.001994 | 0.002148 | 0.00145 | 0.000443 | 0.00398 | 0.000568 | 0.001303 | 0.001101 |
| extraTrees27 | 0.002009 | 0.001034 | 0.001378 | 0.001981 | 0.00146 | 0.001237 | 0.005339 | 0.003722 | 0 | 0.000257 |
| gbc0 | 0.000293 | 9.72E−07 | 0 | 0.000256 | 0 | 0 | 0.004063 | 0 | 0 | 0 |
| gbc1 | 0.000702 | 0 | 0 | 0 | 5.89E−05 | 0.000507 | 0 | 0 | 1.65E−07 | 0 |
| gbc2 | 0.008543 | 0 | 1.88E−05 | 0.003108 | 0.001462 | 0 | 0 | 0 | 0 | 0 |
| gbc3 | 1.93E−05 | 0 | 0 | 0.006407 | 0.004034 | 0.005485 | 0.000235 | 0 | 0 | 5.91E−05 |
| gbc4 | 0 | 0 | 0 | 0 | 0 | 0 | 0.002317 | 3.27E−06 | 0 | 0 |
| gbc5 | 0.000558 | 0 | 3.52E−06 | 0.005823 | 0.000396 | 0 | 0.000526 | 3.86E−05 | 0 | 0 |
| gbc6 | 0.002119 | 0 | 0 | 0.00153 | 0 | 0 | 0 | 0.012728 | 0 | 0 |
| gbc7 | 0 | 0 | 0.000288 | 6.11E−06 | 8.79E−05 | 0.000331 | 0 | 0 | 0 | 0.000159 |
| gbc8 | 1.10E−05 | 1.36E−06 | 0.000258 | 0.000347 | 2.30E−05 | 1.95E−05 | 0.000233 | 0 | 0 | 0 |
| gbc9 | 0.02134 | 0 | 0.000381 | 0 | 0 | 0 | 7.47E−05 | 2.36E−05 | 0.000495 | 0 |
| gbc10 | 0.034462 | 0.000231 | 0 | 0.001043 | 4.85E−05 | 0 | 8.61E−06 | 0 | 0 | 5.75E−06 |
| gbc11 | 0.000451 | 0 | 0.000115 | 0 | 0 | 3.28E−05 | 0 | 0 | 0 | 0 |
| gbc12 | 0.017015 | 0.000108 | 0 | 0 | 0 | 3.56E−06 | 0.003104 | 0.003894 | 0 | 0 |
| gbc13 | 1.07E−05 | 0 | 1.60E−05 | 5.81E−06 | 0.000231 | 0 | 0 | 0 | 9.75E−09 | 0 |
| gbc14 | 0.000293 | 9.72E−07 | 0 | 0.000256 | 0 | 0 | 0.004063 | 0 | 0 | 0 |
| gbc15 | 0.000702 | 0 | 0 | 0 | 5.89E−05 | 0.000507 | 0 | 0 | 1.65E−07 | 0 |
| gbc16 | 0.008543 | 0 | 1.88E−05 | 0.003108 | 0.001462 | 0 | 0 | 0 | 0 | 0 |
| gbc17 | 1.93E−05 | 0 | 0 | 0.006407 | 0.004034 | 0.005485 | 0.000235 | 0 | 0 | 5.91E−05 |
| gbc18 | 0 | 0 | 0 | 0 | 0 | 0 | 0.002317 | 3.27E−06 | 0 | 0 |
| gbc19 | 0.000558 | 0 | 3.52E−06 | 0.005823 | 0.000396 | 0 | 0.000526 | 3.86E−05 | 0 | 0 |
| gbc20 | 0.002119 | 0 | 0 | 0.00153 | 0 | 0 | 0 | 0.012728 | 0 | 0 |
| gbc21 | 0 | 0 | 0.000288 | 6.11E−06 | 8.79E−05 | 0.000331 | 0 | 0 | 0 | 0.000159 |
| gbc22 | 1.10E−05 | 1.36E−06 | 0.000258 | 0.000347 | 2.30E−05 | 1.95E−05 | 0.000233 | 0 | 0 | 0 |
| gbc23 | 0.02134 | 0 | 0.000381 | 0 | 0 | 0 | 7.47E−05 | 2.36E−05 | 0.000495 | 0 |
| gbc24 | 0.034462 | 0.000231 | 0 | 0.001043 | 4.85E−05 | 0 | 8.61E−06 | 0 | 0 | 5.75E−06 |
| gbc25 | 0.000451 | 0 | 0.000115 | 0 | 0 | 3.28E−05 | 0 | 0 | 0 | 0 |
| gbc26 | 0.017015 | 0.000108 | 0 | 0 | 0 | 3.56E−06 | 0.003104 | 0.003894 | 0 | 0 |
| gbc27 | 1.07E−05 | 0 | 1.60E−05 | 5.81E−06 | 0.000231 | 0 | 0 | 0 | 9.75E−09 | 0 |
| ada0 | 0 | 0 | 0 | 0 | 0 | 0 | 0.02 | 0 | 0 | 0 |
| ada1 | 0 | 0 | 0 | 0 | 0 | 0 | 0.02 | 0 | 0 | 0 |
| ada2 | 0 | 0 | 0 | 0 | 0 | 0 | 0.02 | 0 | 0 | 0 |
| ada3 | 0 | 0 | 0 | 0 | 0 | 0 | 0.02 | 0 | 0 | 0 |
| ada4 | 0 | 0 | 0 | 0 | 0 | 0 | 0.02 | 0 | 0 | 0 |
| ada5 | 0 | 0 | 0 | 0 | 0 | 0 | 0.02 | 0 | 0 | 0 |
| ada6 | 0 | 0 | 0 | 0 | 0 | 0 | 0.02 | 0 | 0 | 0 |
| ada7 | 0 | 0 | 0 | 0 | 0 | 0 | 0.02 | 0 | 0 | 0 |
| ada8 | 0 | 0 | 0 | 0 | 0 | 0 | 0.02 | 0 | 0 | 0 |
| ada9 | 0 | 0 | 0 | 0 | 0 | 0 | 0.02 | 0 | 0 | 0 |
| ada10 | 0 | 0 | 0 | 0 | 0 | 0 | 0.02 | 0 | 0 | 0 |
| ada11 | 0 | 0 | 0 | 0 | 0 | 0 | 0.02 | 0 | 0 | 0 |
| ada12 | 0 | 0 | 0 | 0 | 0 | 0 | 0.02 | 0 | 0 | 0 |
| ada13 | 0 | 0 | 0 | 0 | 0 | 0 | 0.02 | 0 | 0 | 0 |
| ada14 | 0 | 0 | 0 | 0 | 0 | 0 | 0.02 | 0 | 0 | 0 |
| ada15 | 0 | 0 | 0 | 0 | 0 | 0 | 0.02 | 0 | 0 | 0 |
| ada16 | 0 | 0 | 0 | 0 | 0 | 0 | 0.02 | 0 | 0 | 0 |
| ada17 | 0 | 0 | 0 | 0 | 0 | 0 | 0.02 | 0 | 0 | 0 |
| ada18 | 0 | 0 | 0 | 0 | 0 | 0 | 0.02 | 0 | 0 | 0 |
| ada19 | 0 | 0 | 0 | 0 | 0 | 0 | 0.02 | 0 | 0 | 0 |
| ada20 | 0 | 0 | 0 | 0 | 0 | 0 | 0.02 | 0 | 0 | 0 |
| ada21 | 0 | 0 | 0 | 0 | 0 | 0 | 0.02 | 0 | 0 | 0 |
| ada22 | 0 | 0 | 0 | 0 | 0 | 0 | 0.02 | 0 | 0 | 0 |
| ada23 | 0 | 0 | 0 | 0 | 0 | 0 | 0.02 | 0 | 0 | 0 |
| ada24 | 0 | 0 | 0 | 0 | 0 | 0 | 0.02 | 0 | 0 | 0 |
| ada25 | 0 | 0 | 0 | 0 | 0 | 0 | 0.02 | 0 | 0 | 0 |
| ada26 | 0 | 0 | 0 | 0 | 0 | 0 | 0.02 | 0 | 0 | 0 |
| ada27 | 0 | 0 | 0 | 0 | 0 | 0 | 0.02 | 0 | 0 | 0 |

| Gene | CLK1 | CNOT7 | COBLL1 | COCH | COLEC12 | COPS2 | COQ10B | CREB1 | CRLF3 | CROT |
|---|---|---|---|---|---|---|---|---|---|---|
| rf0 | 0 | 0.000285 | 0.001301 | 0.002038 | 0.00213 | 0.001 | 0.000817 | 0.000933 | 0.002284 | 0.002502 |
| rf1 | 0.002221 | 0.000304 | 0.002157 | 0 | 0.002493 | 0.002735 | 0.000748 | 0.008668 | 0.003222 | 0.001311 |
| rf2 | 0 | 0 | 0.00851 | 0.003405 | 0 | 0 | 0.005035 | 0.007281 | 0.000749 | 0.001772 |
| rf3 | 0 | 0 | 0.004891 | 0.003328 | 0.000689 | 0.00151 | 0.001675 | 0.000696 | 0 | 0.000634 |
| rf4 | 0.002313 | 0 | 0.005929 | 0 | 0.00033 | 0.001571 | 0.006111 | 0.003996 | 0.001065 | 0 |
| rf5 | 0.004092 | 9.13E−18 | 0.002813 | 0.002156 | 0 | 0.001591 | 0.001283 | 0.005484 | 0 | 3.68E−18 |
| rf6 | 0.004762 | 0.004914 | 0 | 0.001389 | 0 | 0.001735 | 0.001395 | 0.000293 | 0.003041 | 0.000343 |
| rf7 | 0.001133 | 0 | 0.000996 | 0.001385 | 0.002453 | 0.003919 | 0.001834 | 0 | 0.00158 | 0.003187 |
| rf8 | 0.004495 | 0.00035 | 0.001483 | 0.000731 | 0.001094 | 0.000659 | 0.000515 | 0.001288 | 0.004923 | 0 |
| rf9 | 0.001892 | 0 | 0.001358 | 0.000459 | 0.004656 | 2.21E−18 | 0.000599 | 0 | 0.00156 | 0.001135 |
| rf10 | 0.000642 | 0.003906 | 0.002323 | 4.98E−18 | 0.000703 | 0.001011 | 0 | 0.000554 | 0.006844 | 0 |
| rf11 | 0.006086 | 0 | 0.002633 | 0.005182 | 0.006018 | 0.001859 | 0 | 0.004941 | 0.001572 | 0.009252 |
| rf12 | 0 | 3.07E−18 | 0.001384 | 0.001571 | 0 | 0 | 0.001578 | 0.006662 | 0.001046 | 0.000485 |
| rf13 | 0.000724 | 0.004325 | 0.004305 | 0.001222 | 0.000363 | 0 | 0.003188 | 0.007973 | 0 | 0.005683 |
| rf14 | 0 | 0.000285 | 0.001301 | 0.002038 | 0.00213 | 0.001 | 0.000817 | 0.000933 | 0.002284 | 0.002502 |
| rf15 | 0.002221 | 0.000304 | 0.002157 | 0 | 0.002493 | 0.002735 | 0.000748 | 0.008668 | 0.003222 | 0.001311 |
| rf16 | 0 | 0 | 0.00851 | 0.003405 | 0 | 0 | 0.005035 | 0.007281 | 0.000749 | 0.001772 |
| rf17 | 0 | 0 | 0.004891 | 0.003328 | 0.000689 | 0.00151 | 0.001675 | 0.000696 | 0 | 0.000634 |
| rf18 | 0.002313 | 0 | 0.005929 | 0 | 0.00033 | 0.001571 | 0.006111 | 0.003996 | 0.001065 | 0 |
| rf19 | 0.004092 | 9.13E−18 | 0.002813 | 0.002156 | 0 | 0.001591 | 0.001283 | 0.005484 | 0 | 3.68E−18 |
| rf20 | 0.004762 | 0.004914 | 0 | 0.001389 | 0 | 0.001735 | 0.001395 | 0.000293 | 0.003041 | 0.000343 |
| rf21 | 0.001133 | 0 | 0.000996 | 0.001385 | 0.002453 | 0.003919 | 0.001834 | 0 | 0.00158 | 0.003187 |
| rf22 | 0.004495 | 0.00035 | 0.001483 | 0.000731 | 0.001094 | 0.000659 | 0.000515 | 0.001288 | 0.004923 | 0 |
| rf23 | 0.001892 | 0 | 0.001358 | 0.000459 | 0.004656 | 2.21E−18 | 0.000599 | 0 | 0.00156 | 0.001135 |
| rf24 | 0.000642 | 0.003906 | 0.002323 | 4.98E−18 | 0.000703 | 0.001011 | 0 | 0.000554 | 0.006844 | 0 |
| rf25 | 0.006086 | 0 | 0.002633 | 0.005182 | 0.006018 | 0.001859 | 0 | 0.004941 | 0.001572 | 0.009252 |
| rf26 | 0 | 3.07E−18 | 0.001384 | 0.001571 | 0 | 0 | 0.001578 | 0.006662 | 0.001046 | 0.000485 |
| rf27 | 0.000724 | 0.004325 | 0.004305 | 0.001222 | 0.000363 | 0 | 0.003188 | 0.007973 | 0 | 0.005683 |
| extraTrees0 | 0.000914 | 0.002288 | 0.002162 | 0.005649 | 0.000516 | 0.001865 | 0.000352 | 0.003968 | 0.002609 | 0.005019 |
| extraTrees1 | 0.003298 | 0.003292 | 0.001571 | 0.000664 | 0.000332 | 0.002935 | 0.001093 | 0.001429 | 0.00028 | 0.000746 |
| extraTrees2 | 0.003033 | 0.00177 | 0.001065 | 0.003037 | 0.002088 | 0.000224 | 0.000295 | 0.001052 | 0.001645 | 0.001435 |
| extraTrees3 | 0.000596 | 0.001213 | 0.002648 | 0.004253 | 0.001496 | 0.003771 | 0.002494 | 0.00219 | 0.000854 | 0 |
| extraTrees4 | 0.003712 | 0.00167 | 0.000801 | 0.004242 | 0.000476 | 0.001129 | 0.000297 | 0.000998 | 0.001929 | 0.000717 |
| extraTrees5 | 0.002678 | 0.000827 | 0.000696 | 0.002111 | 0.001483 | 0.000387 | 0.000817 | 0.002029 | 0.000268 | 0.002254 |
| extraTrees6 | 0.00056 | 0.002296 | 0.000742 | 0.003466 | 0.000492 | 0.001057 | 0 | 0.002012 | 0.0023 | 0.000851 |
| extraTrees7 | 0.006258 | 0.000777 | 0.000912 | 0.000973 | 0.001012 | 0.000492 | 0.000492 | 0.001352 | 4.13E−18 | 0.002922 |
| extraTrees8 | 9.87E−19 | 0.000277 | 0.00169 | 0.001815 | 0 | 0.0029 | 0 | 0.001751 | 0.000565 | 0.002034 |
| extraTrees9 | 0.000388 | 0.001117 | 0.002545 | 0.001444 | 0.001242 | 0.000787 | 0.001206 | 0.001761 | 0.002189 | 0.000238 |
| extraTrees10 | 0.001548 | 0.002416 | 0 | 0.000555 | 0.000246 | 0.001994 | 1.16E−18 | 0.003605 | 0.001027 | 0.000641 |
| extraTrees11 | 0.000567 | 0.001869 | 0.001345 | 0.005733 | 0.000267 | 0.002316 | 0.000629 | 0.003758 | 0.001213 | 0.000537 |
| extraTrees12 | 0 | 0.002272 | 0.000534 | 0.002769 | 0.000369 | 0.000846 | 0.001352 | 0.000268 | 0.000833 | 0.001351 |
| extraTrees13 | 0.000641 | 1.26E−18 | 0.0039 | 0.003307 | 0.001099 | 0.001012 | 0.001066 | 0.001719 | 0.001663 | 0.000697 |
| extraTrees14 | 0.000914 | 0.002288 | 0.002162 | 0.005649 | 0.000516 | 0.001865 | 0.000352 | 0.003968 | 0.002609 | 0.005019 |
| extraTrees15 | 0.003298 | 0.003292 | 0.001571 | 0.000664 | 0.000332 | 0.002935 | 0.001093 | 0.001429 | 0.00028 | 0.000746 |
| extraTrees16 | 0.003033 | 0.00177 | 0.001065 | 0.003037 | 0.002088 | 0.000224 | 0.000295 | 0.001052 | 0.001645 | 0.001435 |
| extraTrees17 | 0.000596 | 0.001213 | 0.002648 | 0.004253 | 0.001496 | 0.003771 | 0.002494 | 0.00219 | 0.000854 | 0 |
| extraTrees18 | 0.003712 | 0.00167 | 0.000801 | 0.004242 | 0.000476 | 0.001129 | 0.000297 | 0.000998 | 0.001929 | 0.000717 |
| extraTrees19 | 0.002678 | 0.000827 | 0.000696 | 0.002111 | 0.001483 | 0.000387 | 0.000817 | 0.002029 | 0.000268 | 0.002254 |
| extraTrees20 | 0.00056 | 0.002296 | 0.000742 | 0.003466 | 0.000492 | 0.001057 | 0 | 0.002012 | 0.0023 | 0.000851 |
| extraTrees21 | 0.006258 | 0.000777 | 0.000912 | 0.000973 | 0.001012 | 0.000492 | 0.000492 | 0.001352 | 4.13E−18 | 0.002922 |
| extraTrees22 | 9.87E−19 | 0.000277 | 0.00169 | 0.001815 | 0 | 0.0029 | 0 | 0.001751 | 0.000565 | 0.002034 |
| extraTrees23 | 0.000388 | 0.001117 | 0.002545 | 0.001444 | 0.001242 | 0.000787 | 0.001206 | 0.001761 | 0.002189 | 0.000238 |
| extraTrees24 | 0.001548 | 0.002416 | 0 | 0.000555 | 0.000246 | 0.001994 | 1.16E−18 | 0.003605 | 0.001027 | 0.000641 |
| extraTrees25 | 0.000567 | 0.001869 | 0.001345 | 0.005733 | 0.000267 | 0.002316 | 0.000629 | 0.003758 | 0.001213 | 0.000537 |
| extraTrees26 | 0 | 0.002272 | 0.000534 | 0.002769 | 0.000369 | 0.000846 | 0.001352 | 0.000268 | 0.000833 | 0.001351 |
| extraTrees27 | 0.000641 | 1.26E−18 | 0.0039 | 0.003307 | 0.001099 | 0.001012 | 0.001066 | 0.001719 | 0.001663 | 0.000697 |
| gbc0 | 1.05E−05 | 0 | 0.000697 | 0.000199 | 0.001656 | 0.005178 | 0.001295 | 0 | 0.012247 | 0 |
| gbc1 | 0 | 0 | 0.002984 | 0 | 2.88E−07 | 0.011734 | 5.86E−05 | 0 | 2.43E−05 | 0 |
| gbc2 | 0 | 0 | 0.001783 | 0.002858 | 0 | 1.65E−07 | 0.00012 | 0 | 0 | 0.021626 |
| gbc3 | 0.004185 | 0 | 3.01E−05 | 0.011869 | 7.27E−05 | 0 | 0 | 0.000173 | 0 | 0 |
| gbc4 | 0.001887 | 5.36E−05 | 0.00096 | 0.003522 | 0 | 4.48E−05 | 0.000146 | 0 | 4.14E−06 | 0.02717 |
| gbc5 | 0.00721 | 0 | 0.002746 | 0.000174 | 0 | 0 | 4.40E−05 | 0 | 0 | 0.000346 |
| gbc6 | 0 | 0 | 0 | 0 | 3.14E−05 | 0 | 6.29E−05 | 0 | 1.58E−05 | 0 |
| gbc7 | 0.001657 | 0 | 0 | 0 | 0 | 0 | 0 | 0 | 0.009395 | 0 |
| gbc8 | 0 | 2.26E−05 | 0 | 0 | 0 | 0.000294 | 0 | 3.08E−05 | 0 | 0 |
| gbc9 | 0 | 0 | 2.74E−05 | 0 | 7.12E−06 | 0 | 0.008061 | 7.18E−05 | 0 | 0 |
| gbc10 | 0.002112 | 0 | 0 | 0 | 0 | 0 | 0 | 0.020577 | 1.08E−05 | 0.000231 |
| gbc11 | 0.000674 | 0 | 0.02034 | 0.008859 | 0 | 0 | 0 | 0 | 0 | 0 |
| gbc12 | 0 | 0 | 0.000315 | 0.000552 | 9.38E−06 | 7.15E−05 | 0 | 0.010982 | 0 | 8.56E−05 |
| gbc13 | 0 | 0 | 2.69E−05 | 0.00012 | 0 | 0.000953 | 0.001638 | 0 | 0 | 0 |
| gbc14 | 1.05E−05 | 0 | 0.000697 | 0.000199 | 0.001656 | 0.005178 | 0.001295 | 0 | 0.012247 | 0 |
| gbc15 | 0 | 0 | 0.002984 | 0 | 2.88E−07 | 0.011734 | 5.86E−05 | 0 | 2.43E−05 | 0 |
| gbc16 | 0 | 0 | 0.001783 | 0.002858 | 0 | 1.65E−07 | 0.00012 | 0 | 0 | 0.021626 |
| gbc17 | 0.004185 | 0 | 3.01E−05 | 0.011869 | 7.27E−05 | 0 | 0 | 0.000173 | 0 | 0 |
| gbc18 | 0.001887 | 5.36E−05 | 0.00096 | 0.003522 | 0 | 4.48E−05 | 0.000146 | 0 | 4.14E−06 | 0.02717 |
| gbc19 | 0.00721 | 0 | 0.002746 | 0.000174 | 0 | 0 | 4.40E−05 | 0 | 0 | 0.000346 |
| gbc20 | 0 | 0 | 0 | 0 | 3.14E−05 | 0 | 6.29E−05 | 0 | 1.58E−05 | 0 |
| gbc21 | 0.001657 | 0 | 0 | 0 | 0 | 0 | 0 | 0 | 0.009395 | 0 |
| gbc22 | 0 | 2.26E−05 | 0 | 0 | 0 | 0.000294 | 0 | 3.08E−05 | 0 | 0 |
| gbc23 | 0 | 0 | 2.74E−05 | 0 | 7.12E−06 | 0 | 0.008061 | 7.18E−05 | 0 | 0 |
| gbc24 | 0.002112 | 0 | 0 | 0 | 0 | 0 | 0 | 0.020577 | 1.08E−05 | 0.000231 |
| gbc25 | 0.000674 | 0 | 0.02034 | 0.008859 | 0 | 0 | 0 | 0 | 0 | 0 |
| gbc26 | 0 | 0 | 0.000315 | 0.000552 | 9.38E−06 | 7.15E−05 | 0 | 0.010982 | 0 | 8.56E−05 |
| gbc27 | 0 | 0 | 2.69E−05 | 0.00012 | 0 | 0.000953 | 0.001638 | 0 | 0 | 0 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | CUL1 | CUX1 | CXXC4 | DAG1 | DCAF16 | DCTN6 | DCUN1D4 | DDX6 | DENND4A | DIS3L2 |
|---|---|---|---|---|---|---|---|---|---|---|
| rf0 | 0.004616 | 0 | 0.001186 | 0 | 0.003582 | 0.001155 | 0.001723 | 0.00495 | 0.002661 | 0.001671 |
| rf1 | 0 | 0.007339 | 0 | 0.000816 | 0.002861 | 0.001089 | 0 | 0.002725 | 0 | 0 |
| rf2 | 0.001506 | 0 | 0.005661 | 0.003507 | 0.000366 | 0 | 0.000995 | 0.000944 | 0.005013 | 0.000744 |
| rf3 | 0 | 0.000822 | 0.003592 | 0 | 0.002346 | 0.002684 | 0 | 0.000577 | 3.06E−18 | 0.00813 |
| rf4 | 0.004456 | 0 | 0.007869 | 0.002327 | 0.007937 | 0 | 0 | 0.001026 | 0.002924 | 0.002336 |
| rf5 | 0.002051 | 0.001534 | 0 | 0.002134 | 0 | 0.002169 | 5.65E−17 | 0.001107 | 6.37E−19 | 0 |
| rf6 | 0.004996 | 0.003515 | 0 | 0 | 0.000656 | 0 | 0.001176 | 0.004581 | 0.005599 | 0.000962 |
| rf7 | 0.000825 | 0.000786 | 0.003871 | 0.001036 | 0.001608 | 0.001083 | 0.000953 | 0.001027 | 0.003849 | 0.000727 |
| rf8 | 0.004854 | 0.001953 | 0.006605 | 0.004622 | 0.006879 | 0.002548 | 0.000971 | 0.002541 | 0.006533 | 0.000318 |
| rf9 | 1.28E−17 | 0.001763 | 0.003626 | 0.00033 | 0 | 0.000745 | 0 | 0.006448 | 0.007406 | 0.000392 |
| rf10 | 0.001658 | 0.002977 | 0 | 0.003763 | 0.005289 | 0.001578 | 0.001194 | 0.001249 | 0 | 0.008125 |
| rf11 | 0 | 0.003453 | 0.000951 | 0 | 4.27E−18 | 0.001387 | 0 | 0.001555 | 0.002113 | 0.002802 |
| rf12 | 0.005596 | 0.001804 | 2.48E−19 | 0.002759 | 0 | 0 | 0.004068 | 0.004604 | 0.007168 | 0 |
| rf13 | 1.04E−17 | 0.001934 | 0.006734 | 1.14E−18 | 0 | 0.002391 | 0 | 0.006032 | 0.005018 | 0.005551 |
| rf14 | 0.004616 | 0 | 0.001186 | 0 | 0.003582 | 0.001155 | 0.001723 | 0.00495 | 0.002661 | 0.001671 |
| rf15 | 0 | 0.007339 | 0 | 0.000816 | 0.002861 | 0.001089 | 0 | 0.002725 | 0 | 0 |
| rf16 | 0.001506 | 0 | 0.005661 | 0.003507 | 0.000366 | 0 | 0.000995 | 0.000944 | 0.005013 | 0.000744 |
| rf17 | 0 | 0.000822 | 0.003592 | 0 | 0.002346 | 0.002684 | 0 | 0.000577 | 3.06E−18 | 0.00813 |
| rf18 | 0.004456 | 0 | 0.007869 | 0.002327 | 0.007937 | 0 | 0 | 0.001026 | 0.002924 | 0.002336 |
| rf19 | 0.002051 | 0.001534 | 0 | 0.002134 | 0 | 0.002169 | 5.65E−17 | 0.001107 | 6.37E−19 | 0 |
| rf20 | 0.004996 | 0.003515 | 0 | 0 | 0.000656 | 0 | 0.001176 | 0.004581 | 0.005599 | 0.000962 |
| rf21 | 0.000825 | 0.000786 | 0.003871 | 0.001036 | 0.001608 | 0.001083 | 0.000953 | 0.001027 | 0.003849 | 0.000727 |
| rf22 | 0.004854 | 0.001953 | 0.006605 | 0.004622 | 0.006879 | 0.002548 | 0.000971 | 0.002541 | 0.006533 | 0.000318 |
| rf23 | 1.28E−17 | 0.001763 | 0.003626 | 0.00033 | 0 | 0.000745 | 0 | 0.006448 | 0.007406 | 0.000392 |
| rf24 | 0.001658 | 0.002977 | 0 | 0.003763 | 0.005289 | 0.001578 | 0.001194 | 0.001249 | 0 | 0.008125 |
| rf25 | 0 | 0.003453 | 0.000951 | 0 | 4.27E−18 | 0.001387 | 0 | 0.001555 | 0.002113 | 0.002802 |
| rf26 | 0.005596 | 0.001804 | 2.48E−19 | 0.002759 | 0 | 0 | 0.004068 | 0.004604 | 0.007168 | 0 |
| rf27 | 1.04E−17 | 0.001934 | 0.006734 | 1.14E−18 | 0 | 0.002391 | 0 | 0.006032 | 0.005018 | 0.005551 |
| extraTrees0 | 0.001805 | 0.000666 | 0.004335 | 0.000819 | 0.002756 | 0.000643 | 0.000238 | 0.001452 | 0.005481 | 0.00442 |
| extraTrees1 | 0.000235 | 0.000436 | 0.005216 | 0.002368 | 0.002345 | 0.001668 | 0.002161 | 0.004381 | 0.004002 | 0.003048 |
| extraTrees2 | 0.000388 | 0.000405 | 0.004518 | 0.0011 | 0.002361 | 0.002502 | 0.001087 | 0.00104 | 0.00149 | 0.001246 |
| extraTrees3 | 0.000504 | 0.001798 | 0.001082 | 0.001602 | 0.001991 | 0.002169 | 0.000264 | 0.000983 | 0.003197 | 0.004701 |
| extraTrees4 | 0.000491 | 0.003243 | 0.00502 | 0.002134 | 0.000445 | 9.33E−18 | 0.001522 | 0.003307 | 0.004947 | 0.000781 |
| extraTrees5 | 0.002541 | 0.001607 | 0.001508 | 0.003819 | 0.002281 | 0.001443 | 0.000983 | 0.002921 | 0.00134 | 0.003038 |
| extraTrees6 | 0.001203 | 0.001431 | 0.001351 | 0.001604 | 0.002291 | 0.000777 | 0.001403 | 0.001044 | 0.004139 | 0.001001 |
| extraTrees7 | 0.002896 | 0.000442 | 0.003109 | 0.001236 | 0.00277 | 0.00091 | 0.001253 | 0.000846 | 0.001724 | 0.002302 |
| extraTrees8 | 3.99E−18 | 0.00088 | 0.003874 | 0.001603 | 0.002103 | 0.002198 | 0.001276 | 0.004677 | 0.002513 | 0.002011 |
| extraTrees9 | 0 | 0.000246 | 0.00336 | 0.002281 | 0.001291 | 0.001592 | 0.002382 | 0.003737 | 0.001562 | 0.002232 |
| extraTrees10 | 2.73E−19 | 0.000238 | 0.001036 | 0.002943 | 0.001612 | 0.002577 | 0.000213 | 0.002573 | 0.000476 | 0.004297 |
| extraTrees11 | 0.001001 | 0.002058 | 0.005582 | 0.000806 | 0.00144 | 0.001398 | 0.000499 | 0.000622 | 0.002248 | 0.002464 |
| extraTrees12 | 7.01E−19 | 0.000525 | 0.000839 | 0.001512 | 0.000843 | 0.000463 | 0.001181 | 0.001283 | 0.004126 | 0.002379 |
| extraTrees13 | 0.001867 | 0.003307 | 0.002069 | 0.003571 | 0.000952 | 4.27E−19 | 0.000511 | 0.000538 | 0.001303 | 0.001517 |
| extraTrees14 | 0.001805 | 0.000666 | 0.004335 | 0.000819 | 0.002756 | 0.000643 | 0.000238 | 0.001452 | 0.005481 | 0.00442 |
| extraTrees15 | 0.000235 | 0.000436 | 0.005216 | 0.002368 | 0.002345 | 0.001668 | 0.002161 | 0.004381 | 0.004002 | 0.003048 |
| extraTrees16 | 0.000388 | 0.000405 | 0.004518 | 0.0011 | 0.002361 | 0.002502 | 0.001087 | 0.00104 | 0.00149 | 0.001246 |
| extraTrees17 | 0.000504 | 0.001798 | 0.001082 | 0.001602 | 0.001991 | 0.002169 | 0.000264 | 0.000983 | 0.003197 | 0.004701 |
| extraTrees18 | 0.000491 | 0.003243 | 0.00502 | 0.002134 | 0.000445 | 9.33E−18 | 0.001522 | 0.003307 | 0.004947 | 0.000781 |
| extraTrees19 | 0.002541 | 0.001607 | 0.001508 | 0.003819 | 0.002281 | 0.001443 | 0.000983 | 0.002921 | 0.00134 | 0.003038 |
| extraTrees20 | 0.001203 | 0.001431 | 0.001351 | 0.001604 | 0.002291 | 0.000777 | 0.001403 | 0.001044 | 0.004139 | 0.001001 |
| extraTrees21 | 0.002896 | 0.000442 | 0.003109 | 0.001236 | 0.00277 | 0.00091 | 0.001253 | 0.000846 | 0.001724 | 0.002302 |
| extraTrees22 | 3.99E−18 | 0.00088 | 0.003874 | 0.001603 | 0.002103 | 0.002198 | 0.001276 | 0.004677 | 0.002513 | 0.002011 |
| extraTrees23 | 0 | 0.000246 | 0.00336 | 0.002281 | 0.001291 | 0.001592 | 0.002382 | 0.003737 | 0.001562 | 0.002232 |
| extraTrees24 | 2.73E−19 | 0.000238 | 0.001036 | 0.002943 | 0.001612 | 0.002577 | 0.000213 | 0.002573 | 0.000476 | 0.004297 |
| extraTrees25 | 0.001001 | 0.002058 | 0.005582 | 0.000806 | 0.00144 | 0.001398 | 0.000499 | 0.000622 | 0.002248 | 0.002464 |
| extraTrees26 | 7.01E−19 | 0.000525 | 0.000839 | 0.001512 | 0.000843 | 0.000463 | 0.001181 | 0.001283 | 0.004126 | 0.002379 |
| extraTrees27 | 0.001867 | 0.003307 | 0.002069 | 0.003571 | 0.000952 | 4.27E−19 | 0.000511 | 0.000538 | 0.001303 | 0.001517 |
| gbc0 | 0.000554 | 0.004549 | 0.037351 | 0 | 3.70E−05 | 0 | 0.017814 | 0 | 0.009685 | 0.001901 |
| gbc1 | 0.012273 | 0.000639 | 0.005356 | 0 | 0 | 0 | 0 | 0.004384 | 0.003159 | 0 |
| gbc2 | 0.005445 | 0 | 0.001364 | 4.92E−05 | 0 | 0.000137 | 0.005984 | 5.43E−05 | 0 | 4.34E−05 |
| gbc3 | 0.000634 | 0 | 0 | 4.06E−06 | 0.00037 | 0 | 0 | 0 | 0.010397 | 0.000238 |
| gbc4 | 0.001248 | 1.78E−05 | 0 | 0 | 0 | 0 | 0 | 0 | 8.53E−06 | 0.000109 |
| gbc5 | 0 | 0 | 0.006812 | 0.000607 | 0 | 0 | 5.13E−05 | 0.003171 | 7.41E−05 | 0.000181 |
| gbc6 | 0 | 0.000135 | 8.34E−05 | 0 | 0.000314 | 3.15E−05 | 0.001329 | 5.74E−05 | 0.015277 | 5.68E−05 |
| gbc7 | 4.12E−05 | 0 | 0 | 0 | 0 | 4.73E−05 | 0 | 0 | 0 | 0 |
| gbc8 | 0.000453 | 0.007865 | 0.011973 | 0.004665 | 0 | 1.78E−07 | 0 | 0.010553 | 0.043906 | 0.007533 |
| gbc9 | 0 | 0 | 0.000251 | 0 | 0 | 0.002262 | 0.000694 | 2.97E−05 | 0.022154 | 0.001151 |
| gbc10 | 0 | 0 | 0.000134 | 0 | 0.003025 | 0.002718 | 2.57E−05 | 0.005107 | 0.004805 | 0.000725 |
| gbc11 | 0.013747 | 1.57E−05 | 0.016549 | 0 | 0.012817 | 3.60E−05 | 8.99E−05 | 0.000688 | 0.001319 | 1.13E−05 |
| gbc12 | 0.00179 | 0.000501 | 0 | 0 | 1.20E−06 | 0 | 0.001648 | 9.08E−05 | 0.001068 | 4.93E−05 |
| gbc13 | 0 | 0 | 0.000554 | 1.87E−05 | 0 | 0.001788 | 0 | 0.000193 | 0.000211 | 4.27E−05 |
| gbc14 | 0.000554 | 0.004549 | 0.037351 | 0 | 3.70E−05 | 0 | 0.017814 | 0 | 0.009685 | 0.001901 |
| gbc15 | 0.012273 | 0.000639 | 0.005356 | 0 | 0 | 0 | 0 | 0.004384 | 0.003159 | 0 |
| gbc16 | 0.005445 | 0 | 0.001364 | 4.92E−05 | 0 | 0.000137 | 0.005984 | 5.43E−05 | 0 | 4.34E−05 |
| gbc17 | 0.000634 | 0 | 0 | 4.06E−06 | 0.00037 | 0 | 0 | 0 | 0.010397 | 0.000238 |
| gbc18 | 0.001248 | 1.78E−05 | 0 | 0 | 0 | 0 | 0 | 0 | 8.53E−06 | 0.000109 |
| gbc19 | 0 | 0 | 0.006812 | 0.000607 | 0 | 0 | 5.13E−05 | 0.003171 | 7.41E−05 | 0.000181 |
| gbc20 | 0 | 0.000135 | 8.34E−05 | 0 | 0.000314 | 3.15E−05 | 0.001329 | 5.74E−05 | 0.015277 | 5.68E−05 |
| gbc21 | 4.12E−05 | 0 | 0 | 0 | 0 | 4.73E−05 | 0 | 0 | 0 | 0 |
| gbc22 | 0.000453 | 0.007865 | 0.011973 | 0.004665 | 0 | 1.78E−07 | 0 | 0.010553 | 0.043906 | 0.007533 |
| gbc23 | 0 | 0 | 0.000251 | 0 | 0 | 0.002262 | 0.000694 | 2.97E−05 | 0.022154 | 0.001151 |
| gbc24 | 0 | 0 | 0.000134 | 0 | 0.003025 | 0.002718 | 2.57E−05 | 0.005107 | 0.004805 | 0.000725 |
| gbc25 | 0.013747 | 1.57E−05 | 0.016549 | 0 | 0.012817 | 3.60E−05 | 8.99E−05 | 0.000688 | 0.001319 | 1.13E−05 |
| gbc26 | 0.00179 | 0.000501 | 0 | 0 | 1.20E−06 | 0 | 0.001648 | 9.08E−05 | 0.001068 | 4.93E−05 |
| gbc27 | 0 | 0 | 0.000554 | 1.87E−05 | 0 | 0.001788 | 0 | 0.000193 | 0.000211 | 4.27E−05 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | DNAJB2 | DTWD1 | EDEM3 | EEF1AKMT2 | EIF2AK1 | ELAC1 | ELF1 | ELF2 | EMC2 | EMCN |
|---|---|---|---|---|---|---|---|---|---|---|
| rf0 | 0.003655 | 0.005649 | 0 | 6.42E−18 | 0.004807 | 0.003363 | 0.000714 | 0.00113 | 0.000279 | 0.004302 |
| rf1 | 0.002517 | 0.016788 | 0.001405 | 0.002848 | 0.004131 | 0.005 | 0 | 3.77E−18 | 0.001889 | 0 |
| rf2 | 0 | 0.007275 | 0.001065 | 0 | 0.005243 | 0.00439 | 0.001984 | 0.001134 | 0.001163 | 0.002692 |
| rf3 | 0.001869 | 0.01603 | 0.005428 | 0.003131 | 0.0014 | 0.004806 | 0.000674 | 0.004833 | 0.013355 | 0 |
| rf4 | 0.001332 | 0.003557 | 0.00037 | 9.92E−19 | 0.004672 | 0.008637 | 0.004634 | 0.004568 | 0.001769 | 0.001076 |
| rf5 | 0.001176 | 0.012674 | 0 | 0 | 0.00239 | 0.001152 | 0.000444 | 0 | 0 | 0.002266 |
| rf6 | 0.000315 | 0.001902 | 0.001972 | 0 | 0.004551 | 0.001052 | 0.001419 | 0 | 0.000452 | 0.001736 |
| rf7 | 0.006489 | 0.016064 | 0.000293 | 0.001613 | 0.000719 | 0.003938 | 0.000709 | 0 | 0.000393 | 0.001119 |
| rf8 | 0.000775 | 0.013079 | 0.000409 | 0.000856 | 0.000274 | 0.00204 | 0.000415 | 0.004835 | 0.004673 | 0.002477 |
| rf9 | 0.005057 | 0.002936 | 0.000655 | 0.003658 | 1.61E−18 | 0.002479 | 0 | 0.000656 | 0.001313 | 0 |
| rf10 | 0.000607 | 0.001196 | 0 | 0 | 0.005671 | 0.00036 | 2.99E−18 | 0.001451 | 0.000355 | 7.96E−19 |
| rf11 | 0 | 0.014614 | 0.004377 | 0.000378 | 0.004001 | 0.003401 | 0 | 0 | 0.001057 | 6.86E−05 |
| rf12 | 0.002639 | 0.007465 | 0 | 0.000947 | 0.004841 | 0.004117 | 0 | 0.000371 | 0.005406 | 0 |
| rf13 | 0.013696 | 0.00849 | 0.001301 | 0.000807 | 0.000671 | 0.003692 | 0 | 0.00295 | 0 | 1.10E−17 |
| rf14 | 0.003655 | 0.005649 | 0 | 6.42E−18 | 0.004807 | 0.003363 | 0.000714 | 0.00113 | 0.000279 | 0.004302 |
| rf15 | 0.002517 | 0.016788 | 0.001405 | 0.002848 | 0.004131 | 0.005 | 0 | 3.77E−18 | 0.001889 | 0 |
| rf16 | 0 | 0.007275 | 0.001065 | 0 | 0.005243 | 0.00439 | 0.001984 | 0.001134 | 0.001163 | 0.002692 |
| rf17 | 0.001869 | 0.01603 | 0.005428 | 0.003131 | 0.0014 | 0.004806 | 0.000674 | 0.004833 | 0.013355 | 0 |
| rf18 | 0.001332 | 0.003557 | 0.00037 | 9.92E−19 | 0.004672 | 0.008637 | 0.004634 | 0.004568 | 0.001769 | 0.001076 |
| rf19 | 0.001176 | 0.012674 | 0 | 0 | 0.00239 | 0.001152 | 0.000444 | 0 | 0 | 0.002266 |
| rf20 | 0.000315 | 0.001902 | 0.001972 | 0 | 0.004551 | 0.001052 | 0.001419 | 0 | 0.000452 | 0.001736 |
| rf21 | 0.006489 | 0.016064 | 0.000293 | 0.001613 | 0.000719 | 0.003938 | 0.000709 | 0 | 0.000393 | 0.001119 |
| rf22 | 0.000775 | 0.013079 | 0.000409 | 0.000856 | 0.000274 | 0.00204 | 0.000415 | 0.004835 | 0.004673 | 0.002477 |
| rf23 | 0.005057 | 0.002936 | 0.000655 | 0.003658 | 1.61E−18 | 0.002479 | 0 | 0.000656 | 0.001313 | 0 |
| rf24 | 0.000607 | 0.001196 | 0 | 0 | 0.005671 | 0.00036 | 2.99E−18 | 0.001451 | 0.000355 | 7.96E−19 |
| rf25 | 0 | 0.014614 | 0.004377 | 0.000378 | 0.004001 | 0.003401 | 0 | 0 | 0.001057 | 6.86E−05 |
| rf26 | 0.002639 | 0.007465 | 0 | 0.000947 | 0.004841 | 0.004117 | 0 | 0.000371 | 0.005406 | 0 |
| rf27 | 0.013696 | 0.00849 | 0.001301 | 0.000807 | 0.000671 | 0.003692 | 0 | 0.00295 | 0 | 1.10E−17 |
| extraTrees0 | 0.001383 | 0.008526 | 0.0003 | 0.001726 | 0.00028 | 0.001305 | 0.003093 | 0.000621 | 0.000736 | 0.00213 |
| extraTrees1 | 0.001027 | 0.004722 | 0.00133 | 0.000796 | 0.000496 | 0.000914 | 0.001315 | 0.002973 | 0.000785 | 0.000771 |
| extraTrees2 | 1.71E−18 | 0.005031 | 0.00067 | 0.00316 | 0.002216 | 0.004139 | 0.0008 | 0.001195 | 5.53E−18 | 0.003611 |
| extraTrees3 | 0.001508 | 0.015457 | 0.001461 | 0.00049 | 0.005202 | 0.003865 | 0.001524 | 0.00288 | 0.002621 | 0.003506 |
| extraTrees4 | 0.001996 | 0.003443 | 0.001419 | 0.002001 | 0.00329 | 0.001923 | 0.000924 | 0.001175 | 0.001268 | 0.002243 |
| extraTrees5 | 0.003766 | 0.011866 | 0.001372 | 0.001239 | 0.00161 | 0.003999 | 0.000772 | 0.001246 | 0.000499 | 0.001083 |
| extraTrees6 | 0.003034 | 0.002043 | 0.002141 | 5.38E−05 | 0.002345 | 0.001777 | 0.002793 | 0.000466 | 0.001439 | 0.002074 |
| extraTrees7 | 0.001995 | 0.00387 | 0.002286 | 0.001664 | 0.006949 | 0.009252 | 0.002207 | 0.002029 | 0 | 0.003179 |
| extraTrees8 | 0.003692 | 0.002551 | 0.00188 | 5.48E−05 | 0.001128 | 0.001536 | 0.001404 | 0.001245 | 0.000101 | 0.001382 |
| extraTrees9 | 0.00153 | 0.003869 | 0.001387 | 0.002128 | 0.002588 | 0.005955 | 0.001783 | 0.001404 | 0.00242 | 0.000732 |
| extraTrees10 | 0.001233 | 0.006792 | 2.05E−19 | 0.000291 | 0.005521 | 0.003327 | 0.002024 | 0.000238 | 0.000802 | 0.001552 |
| extraTrees11 | 0.000711 | 0.005904 | 0.000874 | 0.001808 | 0.00389 | 0.001981 | 0.001066 | 0.001296 | 0.000691 | 0.002546 |
| extraTrees12 | 0.003154 | 0.005816 | 0.001522 | 0.001723 | 0.002428 | 0.00257 | 0.000267 | 0.001106 | 0.002475 | 0.004793 |
| extraTrees13 | 0.00148 | 0.001281 | 0.001332 | 0.000371 | 0.001927 | 0.00423 | 0.002062 | 0.002504 | 0.002178 | 0.001779 |
| extraTrees14 | 0.001383 | 0.008526 | 0.0003 | 0.001726 | 0.00028 | 0.001305 | 0.003093 | 0.000621 | 0.000736 | 0.00213 |
| extraTrees15 | 0.001027 | 0.004722 | 0.00133 | 0.000796 | 0.000496 | 0.000914 | 0.001315 | 0.002973 | 0.000785 | 0.000771 |
| extraTrees16 | 1.71E−18 | 0.005031 | 0.00067 | 0.00316 | 0.002216 | 0.004139 | 0.0008 | 0.001195 | 5.53E−18 | 0.003611 |
| extraTrees17 | 0.001508 | 0.015457 | 0.001461 | 0.00049 | 0.005202 | 0.003865 | 0.001524 | 0.00288 | 0.002621 | 0.003506 |
| extraTrees18 | 0.001996 | 0.003443 | 0.001419 | 0.002001 | 0.00329 | 0.001923 | 0.000924 | 0.001175 | 0.001268 | 0.002243 |
| extraTrees19 | 0.003766 | 0.011866 | 0.001372 | 0.001239 | 0.00161 | 0.003999 | 0.000772 | 0.001246 | 0.000499 | 0.001083 |
| extraTrees20 | 0.003034 | 0.002043 | 0.002141 | 5.38E−05 | 0.002345 | 0.001777 | 0.002793 | 0.000466 | 0.001439 | 0.002074 |
| extraTrees21 | 0.001995 | 0.00387 | 0.002286 | 0.001664 | 0.006949 | 0.009252 | 0.002207 | 0.002029 | 0 | 0.003179 |
| extraTrees22 | 0.003692 | 0.002551 | 0.00188 | 5.48E−05 | 0.001128 | 0.001536 | 0.001404 | 0.001245 | 0.000101 | 0.001382 |
| extraTrees23 | 0.00153 | 0.003869 | 0.001387 | 0.002128 | 0.002588 | 0.005955 | 0.001783 | 0.001404 | 0.00242 | 0.000732 |
| extraTrees24 | 0.001233 | 0.006792 | 2.05E−19 | 0.000291 | 0.005521 | 0.003327 | 0.002024 | 0.000238 | 0.000802 | 0.001552 |
| extraTrees25 | 0.000711 | 0.005904 | 0.000874 | 0.001808 | 0.00389 | 0.001981 | 0.001066 | 0.001296 | 0.000691 | 0.002546 |
| extraTrees26 | 0.003154 | 0.005816 | 0.001522 | 0.001723 | 0.002428 | 0.00257 | 0.000267 | 0.001106 | 0.002475 | 0.004793 |
| extraTrees27 | 0.00148 | 0.001281 | 0.001332 | 0.000371 | 0.001927 | 0.00423 | 0.002062 | 0.002504 | 0.002178 | 0.001779 |
| gbc0 | 0.00349 | 0.015587 | 0.00302 | 0.006829 | 0 | 0 | 0 | 0 | 0.000321 | 0.00053 |
| gbc1 | 0.00557 | 0.021076 | 8.16E−05 | 0 | 0.013811 | 0 | 0 | 0 | 1.85E−06 | 0 |
| gbc2 | 6.24E−05 | 0 | 0 | 0.00152 | 0 | 0.002954 | 0 | 0 | 0.00497 | 0 |
| gbc3 | 7.76E−06 | 0.005568 | 0.009994 | 0 | 0 | 0 | 0.007314 | 6.33E−05 | 4.87E−05 | 0 |
| gbc4 | 0 | 0.021572 | 0 | 3.25E−05 | 0 | 0.000204 | 0.004068 | 0 | 0 | 0 |
| gbc5 | 0 | 1.41E−05 | 0 | 0.001128 | 0.00803 | 0 | 6.07E−06 | 0.000772 | 3.57E−05 | 0 |
| gbc6 | 0.000243 | 0.024684 | 0.006208 | 0 | 0 | 0.00032 | 0 | 0 | 1.68E−05 | 0 |
| gbc7 | 3.03E−05 | 4.62E−05 | 0 | 0 | 0 | 0.003886 | 0 | 0 | 0 | 0 |
| gbc8 | 2.04E−06 | 0.00011 | 0 | 0.000109 | 0 | 0.000722 | 0.003684 | 0.007026 | 1.91E−06 | 0.000155 |
| gbc9 | 0.003988 | 0.003305 | 0 | 0 | 0 | 0.005905 | 0 | 0 | 0 | 0 |
| gbc10 | 0.001581 | 0 | 0 | 0.011896 | 2.49E−06 | 0.006484 | 0 | 2.86E−05 | 0 | 0.000118 |
| gbc11 | 3.63E−05 | 0.006663 | 0.000166 | 0.003186 | 0 | 0.000306 | 0.000589 | 0.011801 | 0 | 0.018992 |
| gbc12 | 0.000813 | 2.90E−05 | 0 | 0.007621 | 0.007494 | 0 | 0 | 1.47E−06 | 0 | 3.04E−06 |
| gbc13 | 1.28E−06 | 0.00048 | 0 | 0 | 0 | 0.001562 | 0 | 0.00452 | 2.59E−05 | 0 |
| gbc14 | 0.00349 | 0.015587 | 0.00302 | 0.006829 | 0 | 0 | 0 | 0 | 0.000321 | 0.00053 |
| gbc15 | 0.00557 | 0.021076 | 8.16E−05 | 0 | 0.013811 | 0 | 0 | 0 | 1.85E−06 | 0 |
| gbc16 | 6.24E−05 | 0 | 0 | 0.00152 | 0 | 0.002954 | 0 | 0 | 0.00497 | 0 |
| gbc17 | 7.76E−06 | 0.005568 | 0.009994 | 0 | 0 | 0 | 0.007314 | 6.33E−05 | 4.87E−05 | 0 |
| gbc18 | 0 | 0.021572 | 0 | 3.25E−05 | 0 | 0.000204 | 0.004068 | 0 | 0 | 0 |
| gbc19 | 0 | 1.41E−05 | 0 | 0.001128 | 0.00803 | 0 | 6.07E−06 | 0.000772 | 3.57E−05 | 0 |
| gbc20 | 0.000243 | 0.024684 | 0.006208 | 0 | 0 | 0.00032 | 0 | 0 | 1.68E−05 | 0 |
| gbc21 | 3.03E−05 | 4.62E−05 | 0 | 0 | 0 | 0.003886 | 0 | 0 | 0 | 0 |
| gbc22 | 2.04E−06 | 0.00011 | 0 | 0.000109 | 0 | 0.000722 | 0.003684 | 0.007026 | 1.91E−06 | 0.000155 |
| gbc23 | 0.003988 | 0.003305 | 0 | 0 | 0 | 0.005905 | 0 | 0 | 0 | 0 |
| gbc24 | 0.001581 | 0 | 0 | 0.011896 | 2.49E−06 | 0.006484 | 0 | 2.86E−05 | 0 | 0.000118 |
| gbc25 | 3.63E−05 | 0.006663 | 0.000166 | 0.003186 | 0 | 0.000306 | 0.000589 | 0.011801 | 0 | 0.018992 |
| gbc26 | 0.000813 | 2.90E−05 | 0 | 0.007621 | 0.007494 | 0 | 0 | 1.47E−06 | 0 | 3.04E−06 |
| gbc27 | 1.28E−06 | 0.00048 | 0 | 0 | 0 | 0.001562 | 0 | 0.00452 | 2.59E−05 | 0 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | ENTHD1 | ENY2 | ERCC1 | F11R | FAM13A | FAM229A | FAM76B | FAR1 | FNBP1 | FOXN2 |
|---|---|---|---|---|---|---|---|---|---|---|
| rf0 | 0.001051 | 0 | 0.000336 | 0.004914 | 0 | 0.002261 | 0.002099 | 0 | 0 | 0.000869 |
| rf1 | 0.007933 | 0 | 0 | 0.00259 | 0 | 0.00239 | 0.000362 | 0.002024 | 0.000449 | 0.000252 |
| rf2 | 0.004127 | 0.000426 | 7.13E−19 | 0.005642 | 8.02E−19 | 0.005294 | 0.001691 | 0.000281 | 0 | 7.64E−05 |
| rf3 | 0.002949 | 0.00039 | 0.000701 | 0.002009 | 0.001056 | 0 | 0.005318 | 0.000687 | 0.000366 | 0.000427 |
| rf4 | 0.004149 | 0.003399 | 0.000708 | 0.001166 | 0.005025 | 0 | 0.001913 | 0.001433 | 0 | 0 |
| rf5 | 0.000886 | 0.000838 | 0.001708 | 0.001175 | 0.002404 | 0.000366 | 0.000331 | 0 | 0.003261 | 0.001335 |
| rf6 | 0.003585 | 0.004012 | 0 | 0.001762 | 0.002077 | 0.002534 | 0 | 0 | 0 | 0 |
| rf7 | 0.000624 | 0.001111 | 0.00148 | 0.000996 | 0.00111 | 0 | 0 | 0.001129 | 0.000377 | 0 |
| rf8 | 0 | 0 | 0 | 0.001042 | 0.003012 | 0.00119 | 0.000585 | 0.000691 | 0.000811 | 0.000376 |
| rf9 | 0.001848 | 0 | 0.00454 | 0.003405 | 0 | 0 | 0 | 0.001621 | 0.00033 | 0 |
| rf10 | 0.008724 | 0.003244 | 0.00178 | 0.00207 | 0.001019 | 0 | 0 | 0 | 0.000936 | 0.000378 |
| rf11 | 0 | 0 | 0.003405 | 0.002809 | 0.00178 | 0.001221 | 0 | 0.001175 | 0.001404 | 0.001894 |
| rf12 | 0.000689 | 0.000932 | 3.61E−18 | 0.012096 | 0 | 0 | 0 | 0 | 0.00088 | 4.34E−19 |
| rf13 | 0 | 0.002097 | 0.000725 | 0 | 0.000353 | 0.000849 | 0.000972 | 7.61E−05 | 0 | 0.00035 |
| rf14 | 0.001051 | 0 | 0.000336 | 0.004914 | 0 | 0.002261 | 0.002099 | 0 | 0 | 0.000869 |
| rf15 | 0.007933 | 0 | 0 | 0.00259 | 0 | 0.00239 | 0.000362 | 0.002024 | 0.000449 | 0.000252 |
| rf16 | 0.004127 | 0.000426 | 7.13E−19 | 0.005642 | 8.02E−19 | 0.005294 | 0.001691 | 0.000281 | 0 | 7.64E−05 |
| rf17 | 0.002949 | 0.00039 | 0.000701 | 0.002009 | 0.001056 | 0 | 0.005318 | 0.000687 | 0.000366 | 0.000427 |
| rf18 | 0.004149 | 0.003399 | 0.000708 | 0.001166 | 0.005025 | 0 | 0.001913 | 0.001433 | 0 | 0 |
| rf19 | 0.000886 | 0.000838 | 0.001708 | 0.001175 | 0.002404 | 0.000366 | 0.000331 | 0 | 0.003261 | 0.001335 |
| rf20 | 0.003585 | 0.004012 | 0 | 0.001762 | 0.002077 | 0.002534 | 0 | 0 | 0 | 0 |
| rf21 | 0.000624 | 0.001111 | 0.00148 | 0.000996 | 0.00111 | 0 | 0 | 0.001129 | 0.000377 | 0 |
| rf22 | 0 | 0 | 0 | 0.001042 | 0.003012 | 0.00119 | 0.000585 | 0.000691 | 0.000811 | 0.000376 |
| rf23 | 0.001848 | 0 | 0.00454 | 0.003405 | 0 | 0 | 0 | 0.001621 | 0.00033 | 0 |
| rf24 | 0.008724 | 0.003244 | 0.00178 | 0.00207 | 0.001019 | 0 | 0 | 0 | 0.000936 | 0.000378 |
| rf25 | 0 | 0 | 0.003405 | 0.002809 | 0.00178 | 0.001221 | 0 | 0.001175 | 0.001404 | 0.001894 |
| rf26 | 0.000689 | 0.000932 | 3.61E−18 | 0.012096 | 0 | 0 | 0 | 0 | 0.00088 | 4.34E−19 |
| rf27 | 0 | 0.002097 | 0.000725 | 0 | 0.000353 | 0.000849 | 0.000972 | 7.61E−05 | 0 | 0.00035 |
| extraTrees0 | 0.00046 | 0.000287 | 0.001722 | 0.004046 | 0.000476 | 0.001654 | 0.000893 | 0.000291 | 0.000604 | 0.001784 |
| extraTrees1 | 0.001218 | 0.00129 | 0.001335 | 0.005102 | 0.000107 | 0.001712 | 0.000621 | 0.000955 | 0.000939 | 0.000235 |
| extraTrees2 | 0 | 0.000885 | 0.000696 | 0.000238 | 0.003926 | 0.002224 | 0.001031 | 0.000498 | 0.000446 | 0.000238 |
| extraTrees3 | 0.003086 | 0.001456 | 0.003472 | 0.005382 | 0.001858 | 0.000238 | 0.004134 | 0.000268 | 0.000945 | 0.000561 |
| extraTrees4 | 0.000553 | 0.003076 | 0.003134 | 0.001829 | 0.002836 | 0.000955 | 0.000238 | 0.000599 | 0.000805 | 0.000525 |
| extraTrees5 | 0.003483 | 0.00076 | 0.002527 | 0.003358 | 0.001157 | 0.00181 | 0.002088 | 0.000984 | 0.001179 | 0.000268 |
| extraTrees6 | 0.00028 | 0.00307 | 0.004523 | 0.001103 | 0.000889 | 0.00112 | 0.000253 | 0.001384 | 0.000551 | 0.00169 |
| extraTrees7 | 0.000526 | 1.26E−18 | 0.002603 | 0.002942 | 0.002573 | 0.002025 | 0.00198 | 0.002329 | 0.001435 | 0.000809 |
| extraTrees8 | 0.001242 | 0.00113 | 0.000861 | 0.001056 | 0.002037 | 0.000238 | 0.002151 | 0.000427 | 4.61E−18 | 0 |
| extraTrees9 | 0.005439 | 0.001306 | 0.000486 | 0.002419 | 0.002102 | 0.001938 | 0.002824 | 0.001707 | 0.00298 | 0.00028 |
| extraTrees10 | 0.001954 | 0.002336 | 0.005816 | 0.003665 | 0.002458 | 7.01E−19 | 0.000715 | 0.000558 | 0.000388 | 0.001135 |
| extraTrees11 | 0.002457 | 0.002051 | 0.001707 | 0.003359 | 0.001418 | 0 | 0.000492 | 0.000689 | 0.000763 | 0.00067 |
| extraTrees12 | 0.00371 | 0.001327 | 0.003548 | 0.002021 | 0.002483 | 0.000764 | 0 | 0.002588 | 0.000488 | 0.000295 |
| extraTrees13 | 0.000488 | 0.002131 | 0.000515 | 0.005291 | 0.001653 | 0.002498 | 0.002015 | 0.001415 | 0.001594 | 2.73E−19 |
| extraTrees14 | 0.00046 | 0.000287 | 0.001722 | 0.004046 | 0.000476 | 0.001654 | 0.000893 | 0.000291 | 0.000604 | 0.001784 |
| extraTrees15 | 0.001218 | 0.00129 | 0.001335 | 0.005102 | 0.000107 | 0.001712 | 0.000621 | 0.000955 | 0.000939 | 0.000235 |
| extraTrees16 | 0 | 0.000885 | 0.000696 | 0.000238 | 0.003926 | 0.002224 | 0.001031 | 0.000498 | 0.000446 | 0.000238 |
| extraTrees17 | 0.003086 | 0.001456 | 0.003472 | 0.005382 | 0.001858 | 0.000238 | 0.004134 | 0.000268 | 0.000945 | 0.000561 |
| extraTrees18 | 0.000553 | 0.003076 | 0.003134 | 0.001829 | 0.002836 | 0.000955 | 0.000238 | 0.000599 | 0.000805 | 0.000525 |
| extraTrees19 | 0.003483 | 0.00076 | 0.002527 | 0.003358 | 0.001157 | 0.00181 | 0.002088 | 0.000984 | 0.001179 | 0.000268 |
| extraTrees20 | 0.00028 | 0.00307 | 0.004523 | 0.001103 | 0.000889 | 0.00112 | 0.000253 | 0.001384 | 0.000551 | 0.00169 |
| extraTrees21 | 0.000526 | 1.26E−18 | 0.002603 | 0.002942 | 0.002573 | 0.002025 | 0.00198 | 0.002329 | 0.001435 | 0.000809 |
| extraTrees22 | 0.001242 | 0.00113 | 0.000861 | 0.001056 | 0.002037 | 0.000238 | 0.002151 | 0.000427 | 4.61E−18 | 0 |
| extraTrees23 | 0.005439 | 0.001306 | 0.000486 | 0.002419 | 0.002102 | 0.001938 | 0.002824 | 0.001707 | 0.00298 | 0.00028 |
| extraTrees24 | 0.001954 | 0.002336 | 0.005816 | 0.003665 | 0.002458 | 7.01E−19 | 0.000715 | 0.000558 | 0.000388 | 0.001135 |
| extraTrees25 | 0.002457 | 0.002051 | 0.001707 | 0.003359 | 0.001418 | 0 | 0.000492 | 0.000689 | 0.000763 | 0.00067 |
| extraTrees26 | 0.00371 | 0.001327 | 0.003548 | 0.002021 | 0.002483 | 0.000764 | 0 | 0.002588 | 0.000488 | 0.000295 |
| extraTrees27 | 0.000488 | 0.002131 | 0.000515 | 0.005291 | 0.001653 | 0.002498 | 0.002015 | 0.001415 | 0.001594 | 2.73E−19 |
| gbc0 | 5.45E−05 | 2.32E−05 | 0 | 0.000316 | 0.000114 | 0.00146 | 0 | 3.46E−07 | 0.000179 | 0 |
| gbc1 | 0 | 0 | 0 | 0.006308 | 0.006866 | 0 | 0 | 0 | 0.007789 | 0 |
| gbc2 | 4.65E−05 | 0 | 0 | 0 | 0.000518 | 0.000407 | 4.10E−05 | 0 | 0.001748 | 0 |
| gbc3 | 0.007382 | 0 | 0.010682 | 0 | 0.000304 | 0 | 0 | 0.000174 | 0.000373 | 1.18E−05 |
| gbc4 | 0 | 1.12E−05 | 0.000693 | 3.92E−05 | 0.008723 | 0.002705 | 0 | 0.000828 | 0 | 0 |
| gbc5 | 0 | 0 | 0.000996 | 0 | 0 | 0.007571 | 1.96E−05 | 0.002725 | 2.60E−05 | 4.86E−05 |
| gbc6 | 0.004662 | 0 | 0.010755 | 0 | 0.027092 | 0.001154 | 0 | 0 | 0 | 0 |
| gbc7 | 1.11E−05 | 0.000155 | 0 | 3.01E−05 | 0.000359 | 0 | 0 | 0 | 0 | 0 |
| gbc8 | 0 | 0.000481 | 0.02 | 9.35E−07 | 0.019353 | 8.50E−05 | 0.008111 | 0 | 0 | 0 |
| gbc9 | 0.00042 | 3.28E−05 | 0.000897 | 0.000422 | 0.006292 | 0.007645 | 1.56E−06 | 0.000244 | 4.29E−05 | 0 |
| gbc10 | 0 | 0 | 5.39E−05 | 0 | 0.002357 | 0 | 0.000277 | 0 | 0 | 0 |
| gbc11 | 0 | 0.013 | 1.90E−07 | 0.000451 | 0.000187 | 7.61E−05 | 0 | 0.000481 | 0 | 7.01E−05 |
| gbc12 | 0.00408 | 0.000548 | 0 | 0 | 0.002669 | 0.005246 | 0.000305 | 0 | 0 | 6.50E−05 |
| gbc13 | 0.000473 | 7.91E−05 | 0 | 0 | 0.00899 | 0.00019 | 0 | 0.007202 | 0 | 4.64E−06 |
| gbc14 | 5.45E−05 | 2.32E−05 | 0 | 0.000316 | 0.000114 | 0.00146 | 0 | 3.46E−07 | 0.000179 | 0 |
| gbc15 | 0 | 0 | 0 | 0.006308 | 0.006866 | 0 | 0 | 0 | 0.007789 | 0 |
| gbc16 | 4.65E−05 | 0 | 0 | 0 | 0.000518 | 0.000407 | 4.10E−05 | 0 | 0.001748 | 0 |
| gbc17 | 0.007382 | 0 | 0.010682 | 0 | 0.000304 | 0 | 0 | 0.000174 | 0.000373 | 1.18E−05 |
| gbc18 | 0 | 1.12E−05 | 0.000693 | 3.92E−05 | 0.008723 | 0.002705 | 0 | 0.000828 | 0 | 0 |
| gbc19 | 0 | 0 | 0.000996 | 0 | 0 | 0.007571 | 1.96E−05 | 0.002725 | 2.60E−05 | 4.86E−05 |
| gbc20 | 0.004662 | 0 | 0.010755 | 0 | 0.027092 | 0.001154 | 0 | 0 | 0 | 0 |
| gbc21 | 1.11E−05 | 0.000155 | 0 | 3.01E−05 | 0.000359 | 0 | 0 | 0 | 0 | 0 |
| gbc22 | 0 | 0.000481 | 0.02 | 9.35E−07 | 0.019353 | 8.50E−05 | 0.008111 | 0 | 0 | 0 |
| gbc23 | 0.00042 | 3.28E−05 | 0.000897 | 0.000422 | 0.006292 | 0.007645 | 1.56E−06 | 0.000244 | 4.29E−05 | 0 |
| gbc24 | 0 | 0 | 5.39E−05 | 0 | 0.002357 | 0 | 0.000277 | 0 | 0 | 0 |
| gbc25 | 0 | 0.013 | 1.90E−07 | 0.000451 | 0.000187 | 7.61E−05 | 0 | 0.000481 | 0 | 7.01E−05 |
| gbc26 | 0.00408 | 0.000548 | 0 | 0 | 0.002669 | 0.005246 | 0.000305 | 0 | 0 | 6.50E−05 |
| gbc27 | 0.000473 | 7.91E−05 | 0 | 0 | 0.00899 | 0.00019 | 0 | 0.007202 | 0 | 4.64E−06 |
| ada0 | 0 | 0 | 0 | 0 | 0.02 | 0 | 0 | 0 | 0 | 0 |
| ada1 | 0 | 0 | 0 | 0 | 0.02 | 0 | 0 | 0 | 0 | 0 |
| ada2 | 0 | 0 | 0 | 0 | 0.02 | 0 | 0 | 0 | 0 | 0 |
| ada3 | 0 | 0 | 0 | 0 | 0.02 | 0 | 0 | 0 | 0 | 0 |
| ada4 | 0 | 0 | 0 | 0 | 0.02 | 0 | 0 | 0 | 0 | 0 |
| ada5 | 0 | 0 | 0 | 0 | 0.02 | 0 | 0 | 0 | 0 | 0 |
| ada6 | 0 | 0 | 0 | 0 | 0.02 | 0 | 0 | 0 | 0 | 0 |
| ada7 | 0 | 0 | 0 | 0 | 0.02 | 0 | 0 | 0 | 0 | 0 |
| ada8 | 0 | 0 | 0 | 0 | 0.02 | 0 | 0 | 0 | 0 | 0 |
| ada9 | 0 | 0 | 0 | 0 | 0.02 | 0 | 0 | 0 | 0 | 0 |
| ada10 | 0 | 0 | 0 | 0 | 0.02 | 0 | 0 | 0 | 0 | 0 |
| ada11 | 0 | 0 | 0 | 0 | 0.02 | 0 | 0 | 0 | 0 | 0 |
| ada12 | 0 | 0 | 0 | 0 | 0.02 | 0 | 0 | 0 | 0 | 0 |
| ada13 | 0 | 0 | 0 | 0 | 0.02 | 0 | 0 | 0 | 0 | 0 |
| ada14 | 0 | 0 | 0 | 0 | 0.02 | 0 | 0 | 0 | 0 | 0 |
| ada15 | 0 | 0 | 0 | 0 | 0.02 | 0 | 0 | 0 | 0 | 0 |
| ada16 | 0 | 0 | 0 | 0 | 0.02 | 0 | 0 | 0 | 0 | 0 |
| ada17 | 0 | 0 | 0 | 0 | 0.02 | 0 | 0 | 0 | 0 | 0 |
| ada18 | 0 | 0 | 0 | 0 | 0.02 | 0 | 0 | 0 | 0 | 0 |
| ada19 | 0 | 0 | 0 | 0 | 0.02 | 0 | 0 | 0 | 0 | 0 |
| ada20 | 0 | 0 | 0 | 0 | 0.02 | 0 | 0 | 0 | 0 | 0 |
| ada21 | 0 | 0 | 0 | 0 | 0.02 | 0 | 0 | 0 | 0 | 0 |
| ada22 | 0 | 0 | 0 | 0 | 0.02 | 0 | 0 | 0 | 0 | 0 |
| ada23 | 0 | 0 | 0 | 0 | 0.02 | 0 | 0 | 0 | 0 | 0 |
| ada24 | 0 | 0 | 0 | 0 | 0.02 | 0 | 0 | 0 | 0 | 0 |
| ada25 | 0 | 0 | 0 | 0 | 0.02 | 0 | 0 | 0 | 0 | 0 |
| ada26 | 0 | 0 | 0 | 0 | 0.02 | 0 | 0 | 0 | 0 | 0 |
| ada27 | 0 | 0 | 0 | 0 | 0.02 | 0 | 0 | 0 | 0 | 0 |
| Gene | FRG1 | FTH1P10 | FTH1P11 | FTH1P12 | FTH1P2 | FTH1P20 | FTH1P7 | FTL | FTLP2 | FTLP3 |
| rf0 | 0.001591 | 0 | 0 | 0.00084 | 0.000928 | 0.00951 | 0.012686 | 3.39E−18 | 0.011113 | 0.001142 |
| rf1 | 0 | 0.000783 | 0.002958 | 0 | 0.002156 | 0.000515 | 0.003694 | 1.34E−18 | 0.01362 | 0 |
| rf2 | 0 | 0 | 0.006107 | 0.001588 | 0.000767 | 0.001541 | 0.001207 | 0 | 0.004832 | 0.002478 |
| rf3 | 0.004483 | 0 | 0.001603 | 0.001112 | 4.90E−19 | 0.007712 | 0.020276 | 0 | 0.011493 | 0.001856 |
| rf4 | 0.002325 | 0.003405 | 0.002246 | 0.007616 | 0.001234 | 0.000727 | 0.006769 | 0.003184 | 0.00332 | 0 |
| rf5 | 0 | 0 | 0.001085 | 0.007468 | 0 | 1.79E−18 | 0 | 0.004685 | 0.005607 | 0 |
| rf6 | 0.002315 | 0.005304 | 0 | 0.011164 | 5.85E−18 | 0 | 0.005519 | 0.001589 | 0 | 0.001846 |
| rf7 | 0.000638 | 0.002074 | 0.001903 | 0.01065 | 0 | 7.40E−05 | 0.016035 | 0.002356 | 0.006535 | 0.004928 |
| rf8 | 0 | 0 | 0.003454 | 0.004878 | 0.001848 | 0 | 0.008095 | 0 | 0.003998 | 0.001071 |
| rf9 | 0 | 0.000358 | 0.001434 | 0.001112 | 0.005979 | 0.001293 | 0.001465 | 0 | 0.008825 | 0.008494 |
| rf10 | 0.002377 | 0.002209 | 0.00047 | 0.003863 | 0.001012 | 0.00148 | 0.00189 | 0.000669 | 0.007198 | 0.001877 |
| rf11 | 0 | 0.000672 | 0.003192 | 0.00298 | 0 | 2.42E−19 | 0 | 0.000857 | 0.004492 | 0 |
| rf12 | 0 | 0.003844 | 0.001809 | 0.00523 | 0.001509 | 0.008935 | 0.007876 | 0.001065 | 0 | 0 |
| rf13 | 0.007117 | 0.00698 | 0 | 0.008669 | 0.002064 | 0.000768 | 0.001399 | 0 | 0.003583 | 4.21E−05 |
| rf14 | 0.001591 | 0 | 0 | 0.00084 | 0.000928 | 0.00951 | 0.012686 | 3.39E−18 | 0.011113 | 0.001142 |
| rf15 | 0 | 0.000783 | 0.002958 | 0 | 0.002156 | 0.000515 | 0.003694 | 1.34E−18 | 0.01362 | 0 |
| rf16 | 0 | 0 | 0.006107 | 0.001588 | 0.000767 | 0.001541 | 0.001207 | 0 | 0.004832 | 0.002478 |
| rf17 | 0.004483 | 0 | 0.001603 | 0.001112 | 4.90E−19 | 0.007712 | 0.020276 | 0 | 0.011493 | 0.001856 |
| rf18 | 0.002325 | 0.003405 | 0.002246 | 0.007616 | 0.001234 | 0.000727 | 0.006769 | 0.003184 | 0.00332 | 0 |
| rf19 | 0 | 0 | 0.001085 | 0.007468 | 0 | 1.79E−18 | 0 | 0.004685 | 0.005607 | 0 |
| rf20 | 0.002315 | 0.005304 | 0 | 0.011164 | 5.85E−18 | 0 | 0.005519 | 0.001589 | 0 | 0.001846 |
| rf21 | 0.000638 | 0.002074 | 0.001903 | 0.01065 | 0 | 7.40E−05 | 0.016035 | 0.002356 | 0.006535 | 0.004928 |
| rf22 | 0 | 0 | 0.003454 | 0.004878 | 0.001848 | 0 | 0.008095 | 0 | 0.003998 | 0.001071 |
| rf23 | 0 | 0.000358 | 0.001434 | 0.001112 | 0.005979 | 0.001293 | 0.001465 | 0 | 0.008825 | 0.008494 |
| rf24 | 0.002377 | 0.002209 | 0.00047 | 0.003863 | 0.001012 | 0.00148 | 0.00189 | 0.000669 | 0.007198 | 0.001877 |
| rf25 | 0 | 0.000672 | 0.003192 | 0.00298 | 0 | 2.42E−19 | 0 | 0.000857 | 0.004492 | 0 |
| rf26 | 0 | 0.003844 | 0.001809 | 0.00523 | 0.001509 | 0.008935 | 0.007876 | 0.001065 | 0 | 0 |
| rf27 | 0.007117 | 0.00698 | 0 | 0.008669 | 0.002064 | 0.000768 | 0.001399 | 0 | 0.003583 | 4.21E−05 |
| extraTrees0 | 0.001351 | 0.000555 | 0.002807 | 0.007558 | 0.000674 | 0.001611 | 0.002786 | 0.000485 | 0.002607 | 0.001392 |
| extraTrees1 | 0.00028 | 0.000932 | 0.006752 | 0.003028 | 0.001298 | 0.004365 | 0.008787 | 0.000238 | 0.002673 | 0.002516 |
| extraTrees2 | 0 | 0.000262 | 0.002767 | 0.004936 | 0.004035 | 0.003613 | 0.002653 | 0.001756 | 0.001494 | 0.00088 |
| extraTrees3 | 0.000794 | 0.000746 | 0.004429 | 0.00397 | 0.000361 | 0.003721 | 0.007497 | 0.001285 | 0.005327 | 0.001173 |
| extraTrees4 | 0.001826 | 0.000873 | 0.00055 | 0.004925 | 0.001384 | 0.001943 | 0.006695 | 0.000753 | 0.010429 | 0.003247 |
| extraTrees5 | 0.000916 | 0.00094 | 0.002204 | 0.001254 | 0.00381 | 0.000982 | 0.007344 | 0.000599 | 0.011396 | 0.00519 |
| extraTrees6 | 0.001798 | 0.003288 | 0.00181 | 0.003927 | 0.003156 | 0.001472 | 0.002567 | 0.000819 | 0.003662 | 0.001964 |
| extraTrees7 | 0.001329 | 0.005394 | 0.002181 | 0.00326 | 0.004742 | 0.002545 | 0.004383 | 0.0003 | 0.001413 | 0.001794 |
| extraTrees8 | 0.000879 | 0 | 0.002229 | 0.00291 | 1.26E−18 | 0.004297 | 0.000768 | 0.000454 | 0.005858 | 0.000905 |
| extraTrees9 | 0.001395 | 0.003197 | 0.002872 | 0.000571 | 0.001404 | 0.002125 | 0.007813 | 0.001166 | 0.003539 | 0.001423 |
| extraTrees10 | 0.002332 | 0.002096 | 0.003124 | 0.00876 | 0.000666 | 0.000258 | 0.002506 | 0.003009 | 0.006834 | 0.001701 |
| extraTrees11 | 0.001436 | 0.000313 | 0.003109 | 0.004034 | 0.000617 | 0.002758 | 0.001471 | 0.001199 | 0.001716 | 0.00151 |
| extraTrees12 | 0.001394 | 0.000679 | 0.002865 | 0.003614 | 0.002014 | 0.001319 | 0.005086 | 0.001201 | 0.008175 | 0.003506 |
| extraTrees13 | 0.002519 | 0.000273 | 0.002141 | 0.003211 | 0.002335 | 0.000564 | 0.001478 | 3.19E−18 | 0.003259 | 0.001908 |
| extraTrees14 | 0.001351 | 0.000555 | 0.002807 | 0.007558 | 0.000674 | 0.001611 | 0.002786 | 0.000485 | 0.002607 | 0.001392 |
| extraTrees15 | 0.00028 | 0.000932 | 0.006752 | 0.003028 | 0.001298 | 0.004365 | 0.008787 | 0.000238 | 0.002673 | 0.002516 |
| extraTrees16 | 0 | 0.000262 | 0.002767 | 0.004936 | 0.004035 | 0.003613 | 0.002653 | 0.001756 | 0.001494 | 0.00088 |
| extraTrees17 | 0.000794 | 0.000746 | 0.004429 | 0.00397 | 0.000361 | 0.003721 | 0.007497 | 0.001285 | 0.005327 | 0.001173 |
| extraTrees18 | 0.001826 | 0.000873 | 0.00055 | 0.004925 | 0.001384 | 0.001943 | 0.006695 | 0.000753 | 0.010429 | 0.003247 |
| extraTrees19 | 0.000916 | 0.00094 | 0.002204 | 0.001254 | 0.00381 | 0.000982 | 0.007344 | 0.000599 | 0.011396 | 0.00519 |
| extraTrees20 | 0.001798 | 0.003288 | 0.00181 | 0.003927 | 0.003156 | 0.001472 | 0.002567 | 0.000819 | 0.003662 | 0.001964 |
| extraTrees21 | 0.001329 | 0.005394 | 0.002181 | 0.00326 | 0.004742 | 0.002545 | 0.004383 | 0.0003 | 0.001413 | 0.001794 |
| extraTrees22 | 0.000879 | 0 | 0.002229 | 0.00291 | 1.26E−18 | 0.004297 | 0.000768 | 0.000454 | 0.005858 | 0.000905 |
| extraTrees23 | 0.001395 | 0.003197 | 0.002872 | 0.000571 | 0.001404 | 0.002125 | 0.007813 | 0.001166 | 0.003539 | 0.001423 |
| extraTrees24 | 0.002332 | 0.002096 | 0.003124 | 0.00876 | 0.000666 | 0.000258 | 0.002506 | 0.003009 | 0.006834 | 0.001701 |
| extraTrees25 | 0.001436 | 0.000313 | 0.003109 | 0.004034 | 0.000617 | 0.002758 | 0.001471 | 0.001199 | 0.001716 | 0.00151 |
| extraTrees26 | 0.001394 | 0.000679 | 0.002865 | 0.003614 | 0.002014 | 0.001319 | 0.005086 | 0.001201 | 0.008175 | 0.003506 |
| extraTrees27 | 0.002519 | 0.000273 | 0.002141 | 0.003211 | 0.002335 | 0.000564 | 0.001478 | 3.19E−18 | 0.003259 | 0.001908 |
| gbc0 | 7.28E−05 | 0 | 4.66E−05 | 0 | 7.93E−05 | 0 | 0.030208 | 0 | 0.028377 | 0 |
| gbc1 | 2.06E−05 | 0 | 9.05E−05 | 0 | 3.26E−05 | 2.93E−06 | 0.000575 | 0 | 2.74E−05 | 0.002067 |
| gbc2 | 0 | 0.000771 | 0.001107 | 0.000942 | 4.69E−05 | 0.000102 | 4.76E−05 | 0.003219 | 0.000127 | 0 |
| gbc3 | 0.000304 | 0 | 0 | 3.22E−05 | 0 | 0 | 0 | 0 | 0 | 0 |
| gbc4 | 0.000112 | 1.63E−05 | 0 | 0 | 0 | 7.72E−05 | 0 | 0 | 0.012451 | 9.09E−06 |
| gbc5 | 0 | 0.012586 | 0 | 0 | 0.000841 | 0 | 0 | 0.006118 | 0 | 0 |
| gbc6 | 0.014212 | 0 | 1.77E−05 | 0.023254 | 0.031403 | 0 | 1.97E−05 | 9.32E−05 | 4.48E−05 | 0 |
| gbc7 | 0.001272 | 1.32E−05 | 0 | 0 | 0.00456 | 0 | 0 | 0 | 0.001976 | 2.91E−05 |
| gbc8 | 0 | 0 | 3.37E−05 | 0.020641 | 0 | 0 | 0 | 0.016939 | 0.000716 | 0.005936 |
| gbc9 | 0 | 0 | 0 | 0.010812 | 0.001175 | 0 | 0 | 0.000112 | 0.007855 | 0 |
| gbc10 | 0.010778 | 0 | 7.66E−05 | 0 | 0 | 0.001297 | 0 | 0.001084 | 0.00348 | 0 |
| gbc11 | 0.000149 | 0 | 0 | 0.000155 | 0 | 3.16E−06 | 0 | 0.001879 | 0 | 0.002719 |
| gbc12 | 0 | 0 | 0.006547 | 4.42E−05 | 0.000427 | 6.51E−06 | 0.007682 | 8.27E−05 | 0.006461 | 0 |
| gbc13 | 0 | 1.58E−05 | 0 | 0.019174 | 0 | 1.18E−05 | 0 | 0 | 0 | 1.41E−05 |
| gbc14 | 7.28E−05 | 0 | 4.66E−05 | 0 | 7.93E−05 | 0 | 0.030208 | 0 | 0.028377 | 0 |
| gbc15 | 2.06E−05 | 0 | 9.05E−05 | 0 | 3.26E−05 | 2.93E−06 | 0.000575 | 0 | 2.74E−05 | 0.002067 |
| gbc16 | 0 | 0.000771 | 0.001107 | 0.000942 | 4.69E−05 | 0.000102 | 4.76E−05 | 0.003219 | 0.000127 | 0 |
| gbc17 | 0.000304 | 0 | 0 | 3.22E−05 | 0 | 0 | 0 | 0 | 0 | 0 |
| gbc18 | 0.000112 | 1.63E−05 | 0 | 0 | 0 | 7.72E−05 | 0 | 0 | 0.012451 | 9.09E−06 |
| gbc19 | 0 | 0.012586 | 0 | 0 | 0.000841 | 0 | 0 | 0.006118 | 0 | 0 |
| gbc20 | 0.014212 | 0 | 1.77E−05 | 0.023254 | 0.031403 | 0 | 1.97E−05 | 9.32E−05 | 4.48E−05 | 0 |
| gbc21 | 0.001272 | 1.32E−05 | 0 | 0 | 0.00456 | 0 | 0 | 0 | 0.001976 | 2.91E−05 |
| gbc22 | 0 | 0 | 3.37E−05 | 0.020641 | 0 | 0 | 0 | 0.016939 | 0.000716 | 0.005936 |
| gbc23 | 0 | 0 | 0 | 0.010812 | 0.001175 | 0 | 0 | 0.000112 | 0.007855 | 0 |
| gbc24 | 0.010778 | 0 | 7.66E−05 | 0 | 0 | 0.001297 | 0 | 0.001084 | 0.00348 | 0 |
| gbc25 | 0.000149 | 0 | 0 | 0.000155 | 0 | 3.16E−06 | 0 | 0.001879 | 0 | 0.002719 |
| gbc26 | 0 | 0 | 0.006547 | 4.42E−05 | 0.000427 | 6.51E−06 | 0.007682 | 8.27E−05 | 0.006461 | 0 |
| gbc27 | 0 | 1.58E−05 | 0 | 0.019174 | 0 | 1.18E−05 | 0 | 0 | 0 | 1.41E−05 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | FUNDC2 | FUNDC2P1 | FUNDC2P2 | FURIN | G6PD | GABPB1 | GABRD | GADD45G | GALM | GINM1 |
|---|---|---|---|---|---|---|---|---|---|---|
| rf0 | 0.000607 | 0 | 0 | 0 | 0.000961 | 0.004094 | 0.002547 | 0.000623 | 3.28E−17 | 0.021562 |
| rf1 | 0.001496 | 0.006047 | 0.001225 | 0.008949 | 0.003652 | 0.000682 | 0.004978 | 0.001839 | 0 | 0.003327 |
| rf2 | 0.002914 | 0 | 0.003194 | 2.07E−18 | 0.002035 | 0.000558 | 0.002717 | 0.002976 | 0.001252 | 0.003756 |
| rf3 | 4.15E−05 | 0.00036 | 0.001585 | 0 | 0.000576 | 0 | 0.001484 | 0.00224 | 0 | 0 |
| rf4 | 0 | 0.000692 | 0.000716 | 0 | 0 | 0.001223 | 0.003505 | 0.010961 | 0.000342 | 0.005302 |
| rf5 | 0 | 0.000324 | 0.003013 | 0.002091 | 0.000838 | 0.000676 | 0.005048 | 0.005375 | 0.000659 | 0.012423 |
| rf6 | 0 | 0.005362 | 0.005229 | 0.007863 | 0 | 0.005235 | 0 | 0.000792 | 0.000102 | 0.002947 |
| rf7 | 0.003042 | 0 | 0.00111 | 0.00198 | 0.000351 | 0.001878 | 0.001365 | 0.002949 | 0 | 7.64E−05 |
| rf8 | 0 | 0.001278 | 0 | 0.000372 | 0.000372 | 0.000868 | 0.002719 | 0.005768 | 0.000824 | 0.004025 |
| rf9 | 0.001683 | 0.00034 | 0 | 0 | 0.000389 | 0.002766 | 0.001572 | 0.005829 | 0 | 0.001499 |
| rf10 | 0.001871 | 0 | 0 | 0 | 0 | 0 | 0 | 0.005177 | 0.001592 | 0 |
| rf11 | 0 | 8.76E−19 | 0.000386 | 0.000365 | 0.001389 | 0.000614 | 0.001304 | 0.002127 | 0.000768 | 0.001044 |
| rf12 | 0.00899 | 0.001022 | 0.001036 | 0.002518 | 0 | 0.001935 | 0.001186 | 0 | 0.001494 | 0.000954 |
| rf13 | 0.000986 | 0.005349 | 0.000922 | 0 | 0.003597 | 0 | 0.003087 | 0.007645 | 0.002422 | 0.004824 |
| rf14 | 0.000607 | 0 | 0 | 0 | 0.000961 | 0.004094 | 0.002547 | 0.000623 | 3.28E−17 | 0.021562 |
| rf15 | 0.001496 | 0.006047 | 0.001225 | 0.008949 | 0.003652 | 0.000682 | 0.004978 | 0.001839 | 0 | 0.003327 |
| rf16 | 0.002914 | 0 | 0.003194 | 2.07E−18 | 0.002035 | 0.000558 | 0.002717 | 0.002976 | 0.001252 | 0.003756 |
| rf17 | 4.15E−05 | 0.00036 | 0.001585 | 0 | 0.000576 | 0 | 0.001484 | 0.00224 | 0 | 0 |
| rf18 | 0 | 0.000692 | 0.000716 | 0 | 0 | 0.001223 | 0.003505 | 0.010961 | 0.000342 | 0.005302 |
| rf19 | 0 | 0.000324 | 0.003013 | 0.002091 | 0.000838 | 0.000676 | 0.005048 | 0.005375 | 0.000659 | 0.012423 |
| rf20 | 0 | 0.005362 | 0.005229 | 0.007863 | 0 | 0.005235 | 0 | 0.000792 | 0.000102 | 0.002947 |
| rf21 | 0.003042 | 0 | 0.00111 | 0.00198 | 0.000351 | 0.001878 | 0.001365 | 0.002949 | 0 | 7.64E−05 |
| rf22 | 0 | 0.001278 | 0 | 0.000372 | 0.000372 | 0.000868 | 0.002719 | 0.005768 | 0.000824 | 0.004025 |
| rf23 | 0.001683 | 0.00034 | 0 | 0 | 0.000389 | 0.002766 | 0.001572 | 0.005829 | 0 | 0.001499 |
| rf24 | 0.001871 | 0 | 0 | 0 | 0 | 0 | 0 | 0.005177 | 0.001592 | 0 |
| rf25 | 0 | 8.76E−19 | 0.000386 | 0.000365 | 0.001389 | 0.000614 | 0.001304 | 0.002127 | 0.000768 | 0.001044 |
| rf26 | 0.00899 | 0.001022 | 0.001036 | 0.002518 | 0 | 0.001935 | 0.001186 | 0 | 0.001494 | 0.000954 |
| rf27 | 0.000986 | 0.005349 | 0.000922 | 0 | 0.003597 | 0 | 0.003087 | 0.007645 | 0.002422 | 0.004824 |
| extraTrees0 | 0.000775 | 0.001384 | 0.00251 | 0.001616 | 0.00082 | 0.002043 | 0.003151 | 0.000513 | 0.00058 | 0.006315 |
| extraTrees1 | 0.002764 | 0.00364 | 0.001186 | 0.001429 | 2.87E−18 | 0 | 0.000207 | 0.002611 | 0.001136 | 0.005938 |
| extraTrees2 | 0.000282 | 0.003008 | 2.73E−19 | 0.000281 | 0.001063 | 0.001228 | 0.002345 | 0.001306 | 0.000574 | 0.003415 |
| extraTrees3 | 0.001473 | 0.002371 | 0.002444 | 0.001075 | 0.003428 | 1.79E−18 | 0.001289 | 0.003203 | 0.002478 | 0.006039 |
| extraTrees4 | 0.001731 | 0.002515 | 0.000778 | 0.001244 | 0.000365 | 0.000473 | 0.000961 | 0.003115 | 0.000434 | 0.004686 |
| extraTrees5 | 0.000594 | 0.00071 | 0.002835 | 0.001695 | 0.0016 | 0.001123 | 0.002418 | 0.00276 | 0.000268 | 0.008228 |
| extraTrees6 | 0.003622 | 0.002879 | 0.002632 | 0.003721 | 0.001024 | 0.001483 | 0.000807 | 0.000238 | 0 | 0.002218 |
| extraTrees7 | 0.00028 | 0.001941 | 0.001103 | 0.001165 | 0.000238 | 0.001184 | 1.17E−17 | 0.001941 | 0.001051 | 0.002527 |
| extraTrees8 | 0.00118 | 0.003096 | 0.000267 | 0.001734 | 0.001213 | 0 | 0.001481 | 0.000267 | 0.000657 | 0.002806 |
| extraTrees9 | 0.002415 | 0.000817 | 0.000946 | 0.003985 | 0.00169 | 2.05E−19 | 0.003684 | 0.001824 | 0.002371 | 0.003074 |
| extraTrees10 | 0.001473 | 0.000295 | 0.002176 | 0.00172 | 0 | 0.001091 | 0.001966 | 0.003861 | 0 | 0.003904 |
| extraTrees11 | 0.00065 | 0.003556 | 0.001288 | 0.001865 | 0.002208 | 0.002156 | 0.000478 | 0.001102 | 0.002009 | 0.003237 |
| extraTrees12 | 0.000476 | 0.002768 | 0.00201 | 0.002135 | 0.000886 | 2.69E−05 | 0.002425 | 0.00133 | 0.001372 | 0.001091 |
| extraTrees13 | 0.001042 | 0.000518 | 0.002388 | 0.001156 | 0.000546 | 0.000388 | 0.000848 | 0 | 0.003003 | 0.008026 |
| extraTrees14 | 0.000775 | 0.001384 | 0.00251 | 0.001616 | 0.00082 | 0.002043 | 0.003151 | 0.000513 | 0.00058 | 0.006315 |
| extraTrees15 | 0.002764 | 0.00364 | 0.001186 | 0.001429 | 2.87E−18 | 0 | 0.000207 | 0.002611 | 0.001136 | 0.005938 |
| extraTrees16 | 0.000282 | 0.003008 | 2.73E−19 | 0.000281 | 0.001063 | 0.001228 | 0.002345 | 0.001306 | 0.000574 | 0.003415 |
| extraTrees17 | 0.001473 | 0.002371 | 0.002444 | 0.001075 | 0.003428 | 1.79E−18 | 0.001289 | 0.003203 | 0.002478 | 0.006039 |
| extraTrees18 | 0.001731 | 0.002515 | 0.000778 | 0.001244 | 0.000365 | 0.000473 | 0.000961 | 0.003115 | 0.000434 | 0.004686 |
| extraTrees19 | 0.000594 | 0.00071 | 0.002835 | 0.001695 | 0.0016 | 0.001123 | 0.002418 | 0.00276 | 0.000268 | 0.008228 |
| extraTrees20 | 0.003622 | 0.002879 | 0.002632 | 0.003721 | 0.001024 | 0.001483 | 0.000807 | 0.000238 | 0 | 0.002218 |
| extraTrees21 | 0.00028 | 0.001941 | 0.001103 | 0.001165 | 0.000238 | 0.001184 | 1.17E−17 | 0.001941 | 0.001051 | 0.002527 |
| extraTrees22 | 0.00118 | 0.003096 | 0.000267 | 0.001734 | 0.001213 | 0 | 0.001481 | 0.000267 | 0.000657 | 0.002806 |
| extraTrees23 | 0.002415 | 0.000817 | 0.000946 | 0.003985 | 0.00169 | 2.05E−19 | 0.003684 | 0.001824 | 0.002371 | 0.003074 |
| extraTrees24 | 0.001473 | 0.000295 | 0.002176 | 0.00172 | 0 | 0.001091 | 0.001966 | 0.003861 | 0 | 0.003904 |
| extraTrees25 | 0.00065 | 0.003556 | 0.001288 | 0.001865 | 0.002208 | 0.002156 | 0.000478 | 0.001102 | 0.002009 | 0.003237 |
| extraTrees26 | 0.000476 | 0.002768 | 0.00201 | 0.002135 | 0.000886 | 2.69E−05 | 0.002425 | 0.00133 | 0.001372 | 0.001091 |
| extraTrees27 | 0.001042 | 0.000518 | 0.002388 | 0.001156 | 0.000546 | 0.000388 | 0.000848 | 0 | 0.003003 | 0.008026 |
| gbc0 | 2.67E−05 | 0 | 6.37E−05 | 0 | 0 | 0 | 0.000208 | 0.000223 | 0 | 0.005456 |
| gbc1 | 0.000769 | 0.000739 | 2.94E−05 | 0 | 0 | 0 | 0 | 0.000204 | 0.001473 | 0.003854 |
| gbc2 | 2.82E−05 | 0 | 4.42E−05 | 8.85E−06 | 0 | 0.000257 | 0.010174 | 0.001211 | 0 | 0.009261 |
| gbc3 | 0.01338 | 0 | 3.33E−06 | 0.007416 | 0 | 0.005258 | 0 | 0.001586 | 0 | 0.00381 |
| gbc4 | 0.007171 | 0 | 0 | 0.000382 | 4.54E−05 | 0 | 0 | 0 | 0 | 0.001199 |
| gbc5 | 2.44E−05 | 0.016553 | 0 | 0 | 0 | 0.006554 | 0 | 0.000568 | 1.60E−05 | 0.001022 |
| gbc6 | 0 | 0 | 2.36E−05 | 0.012709 | 0 | 9.09E−07 | 9.19E−05 | 0.00039 | 0 | 0 |
| gbc7 | 5.22E−07 | 0 | 0 | 0 | 0 | 0.016563 | 0 | 0.001381 | 0 | 0.003205 |
| gbc8 | 0 | 0 | 0.017359 | 0 | 0 | 0 | 0 | 0.000268 | 0.000123 | 0.000459 |
| gbc9 | 0 | 0 | 0.000226 | 0.005494 | 0 | 1.80E−05 | 0.002372 | 0.000335 | 0 | 0.000557 |
| gbc10 | 2.27E−06 | 0 | 0 | 9.41E−05 | 7.41E−05 | 0 | 0 | 9.97E−05 | 5.23E−05 | 0.008282 |
| gbc11 | 0 | 0 | 0 | 0 | 0 | 0.001156 | 0.003685 | 0 | 0 | 0.000208 |
| gbc12 | 0 | 0 | 0 | 0 | 0.000474 | 0 | 0.013132 | 0.003344 | 0 | 2.79E−05 |
| gbc13 | 0.001262 | 0 | 0 | 0 | 0.010084 | 0 | 2.89E−05 | 0.0037 | 0 | 0.004304 |
| gbc14 | 2.67E−05 | 0 | 6.37E−05 | 0 | 0 | 0 | 0.000208 | 0.000223 | 0 | 0.005456 |
| gbc15 | 0.000769 | 0.000739 | 2.94E−05 | 0 | 0 | 0 | 0 | 0.000204 | 0.001473 | 0.003854 |
| gbc16 | 2.82E−05 | 0 | 4.42E−05 | 8.85E−06 | 0 | 0.000257 | 0.010174 | 0.001211 | 0 | 0.009261 |
| gbc17 | 0.01338 | 0 | 3.33E−06 | 0.007416 | 0 | 0.005258 | 0 | 0.001586 | 0 | 0.00381 |
| gbc18 | 0.007171 | 0 | 0 | 0.000382 | 4.54E−05 | 0 | 0 | 0 | 0 | 0.001199 |
| gbc19 | 2.44E−05 | 0.016553 | 0 | 0 | 0 | 0.006554 | 0 | 0.000568 | 1.60E−05 | 0.001022 |
| gbc20 | 0 | 0 | 2.36E−05 | 0.012709 | 0 | 9.09E−07 | 9.19E−05 | 0.00039 | 0 | 0 |
| gbc21 | 5.22E−07 | 0 | 0 | 0 | 0 | 0.016563 | 0 | 0.001381 | 0 | 0.003205 |
| gbc22 | 0 | 0 | 0.017359 | 0 | 0 | 0 | 0 | 0.000268 | 0.000123 | 0.000459 |
| gbc23 | 0 | 0 | 0.000226 | 0.005494 | 0 | 1.80E−05 | 0.002372 | 0.000335 | 0 | 0.000557 |
| gbc24 | 2.27E−06 | 0 | 0 | 9.41E−05 | 7.41E−05 | 0 | 0 | 9.97E−05 | 5.23E−05 | 0.008282 |
| gbc25 | 0 | 0 | 0 | 0 | 0 | 0.001156 | 0.003685 | 0 | 0 | 0.000208 |
| gbc26 | 0 | 0 | 0 | 0 | 0.000474 | 0 | 0.013132 | 0.003344 | 0 | 2.79E−05 |
| gbc27 | 0.001262 | 0 | 0 | 0 | 0.010084 | 0 | 2.89E−05 | 0.0037 | 0 | 0.004304 |
| ada0 | 0 | 0 | 0 | 0 | 0 | 0 | 0 | 0.02 | 0 | 0.02 |
| ada1 | 0 | 0 | 0 | 0 | 0 | 0 | 0 | 0.02 | 0 | 0.02 |
| ada2 | 0 | 0 | 0 | 0 | 0 | 0 | 0 | 0.02 | 0 | 0.02 |
| ada3 | 0 | 0 | 0 | 0 | 0 | 0 | 0 | 0.02 | 0 | 0.02 |
| ada4 | 0 | 0 | 0 | 0 | 0 | 0 | 0 | 0.02 | 0 | 0.02 |
| ada5 | 0 | 0 | 0 | 0 | 0 | 0 | 0 | 0.02 | 0 | 0.02 |
| ada6 | 0 | 0 | 0 | 0 | 0 | 0 | 0 | 0.02 | 0 | 0.02 |
| ada7 | 0 | 0 | 0 | 0 | 0 | 0 | 0 | 0.02 | 0 | 0.02 |
| ada8 | 0 | 0 | 0 | 0 | 0 | 0 | 0 | 0.02 | 0 | 0.02 |
| ada9 | 0 | 0 | 0 | 0 | 0 | 0 | 0 | 0.02 | 0 | 0.02 |
| ada10 | 0 | 0 | 0 | 0 | 0 | 0 | 0 | 0.02 | 0 | 0.02 |
| ada11 | 0 | 0 | 0 | 0 | 0 | 0 | 0 | 0.02 | 0 | 0.02 |
| ada12 | 0 | 0 | 0 | 0 | 0 | 0 | 0 | 0.02 | 0 | 0.02 |
| ada13 | 0 | 0 | 0 | 0 | 0 | 0 | 0 | 0.02 | 0 | 0.02 |
| ada14 | 0 | 0 | 0 | 0 | 0 | 0 | 0 | 0.02 | 0 | 0.02 |
| ada15 | 0 | 0 | 0 | 0 | 0 | 0 | 0 | 0.02 | 0 | 0.02 |
| ada16 | 0 | 0 | 0 | 0 | 0 | 0 | 0 | 0.02 | 0 | 0.02 |
| ada17 | 0 | 0 | 0 | 0 | 0 | 0 | 0 | 0.02 | 0 | 0.02 |
| ada18 | 0 | 0 | 0 | 0 | 0 | 0 | 0 | 0.02 | 0 | 0.02 |
| ada19 | 0 | 0 | 0 | 0 | 0 | 0 | 0 | 0.02 | 0 | 0.02 |
| ada20 | 0 | 0 | 0 | 0 | 0 | 0 | 0 | 0.02 | 0 | 0.02 |
| ada21 | 0 | 0 | 0 | 0 | 0 | 0 | 0 | 0.02 | 0 | 0.02 |
| ada22 | 0 | 0 | 0 | 0 | 0 | 0 | 0 | 0.02 | 0 | 0.02 |
| ada23 | 0 | 0 | 0 | 0 | 0 | 0 | 0 | 0.02 | 0 | 0.02 |
| ada24 | 0 | 0 | 0 | 0 | 0 | 0 | 0 | 0.02 | 0 | 0.02 |
| ada25 | 0 | 0 | 0 | 0 | 0 | 0 | 0 | 0.02 | 0 | 0.02 |
| ada26 | 0 | 0 | 0 | 0 | 0 | 0 | 0 | 0.02 | 0 | 0.02 |
| ada27 | 0 | 0 | 0 | 0 | 0 | 0 | 0 | 0.02 | 0 | 0.02 |

| Gene | GIPC3 | GLRX3 | GNAI3 | GPAT4 | GPATCH2 | GPBP1 | GRK4 | GSN | GSPT1 | GTPBP8 |
|---|---|---|---|---|---|---|---|---|---|---|
| rf0 | 0.000386 | 0.001764 | 4.96E−19 | 0.005603 | 0.007002 | 8.17E−18 | 0.000353 | 0.001025 | 0.001383 | 0.007208 |
| rf1 | 0.005091 | 0.005273 | 0 | 0.001542 | 0.004279 | 1.17E−18 | 0.001489 | 0.000293 | 0 | 0.00501 |
| rf2 | 0.002498 | 2.56E−19 | 0 | 0.004234 | 0.006462 | 0 | 0.000918 | 0.001331 | 0.001582 | 0.003876 |
| rf3 | 0 | 0 | 4.20E−19 | 0 | 0.009559 | 2.97E−17 | 0.001077 | 0.004332 | 0.000316 | 0.003491 |
| rf4 | 0 | 0.006031 | 5.23E−19 | 0.000988 | 0.002647 | 0 | 0 | 0 | 0.001708 | 0.00106 |
| rf5 | 0.002118 | 0.002564 | 0 | 0 | 0.000978 | 0 | 0 | 0.001152 | 0 | 0 |
| rf6 | 0.004145 | 0.002525 | 0.001456 | 0.002468 | 0.000738 | 2.52E−05 | 0.003286 | 0.001132 | 0.002821 | 0.002197 |
| rf7 | 0.003608 | 0.001715 | 0.004502 | 0.001021 | 0 | 0.00052 | 0.003253 | 0.000995 | 0.005642 | 0.003732 |
| rf8 | 0 | 0.000393 | 0 | 0.001056 | 0.008116 | 0 | 0.002076 | 0.000151 | 0.003014 | 0.004656 |
| rf9 | 0.001142 | 0.002513 | 0.001407 | 0.005453 | 0 | 0 | 0.003421 | 0.001632 | 0.005475 | 0 |
| rf10 | 0.000854 | 0.005357 | 0 | 0.007991 | 0.001054 | 0.000403 | 0.002633 | 0 | 0.00231 | 0.003788 |
| rf11 | 0 | 0.013547 | 0 | 0.008204 | 0 | 0 | 0.003158 | 0.001131 | 0 | 0.000104 |
| rf12 | 0.001066 | 0.003298 | 0.001039 | 0.000972 | 0.004196 | 0.001709 | 0 | 0.000361 | 1.82E−17 | 0.006138 |
| rf13 | 0.000386 | 0.009366 | 0.006817 | 0.001343 | 0.003125 | 0 | 0.000564 | 0.003643 | 0.002373 | 0.007647 |
| rf14 | 0.000386 | 0.001764 | 4.96E−19 | 0.005603 | 0.007002 | 8.17E−18 | 0.000353 | 0.001025 | 0.001383 | 0.007208 |
| rf15 | 0.005091 | 0.005273 | 0 | 0.001542 | 0.004279 | 1.17E−18 | 0.001489 | 0.000293 | 0 | 0.00501 |
| rf16 | 0.002498 | 2.56E−19 | 0 | 0.004234 | 0.006462 | 0 | 0.000918 | 0.001331 | 0.001582 | 0.003876 |
| rf17 | 0 | 0 | 4.20E−19 | 0 | 0.009559 | 2.97E−17 | 0.001077 | 0.004332 | 0.000316 | 0.003491 |
| rf18 | 0 | 0.006031 | 5.23E−19 | 0.000988 | 0.002647 | 0 | 0 | 0 | 0.001708 | 0.00106 |
| rf19 | 0.002118 | 0.002564 | 0 | 0 | 0.000978 | 0 | 0 | 0.001152 | 0 | 0 |
| rf20 | 0.004145 | 0.002525 | 0.001456 | 0.002468 | 0.000738 | 2.52E−05 | 0.003286 | 0.001132 | 0.002821 | 0.002197 |
| rf21 | 0.003608 | 0.001715 | 0.004502 | 0.001021 | 0 | 0.00052 | 0.003253 | 0.000995 | 0.005642 | 0.003732 |
| rf22 | 0 | 0.000393 | 0 | 0.001056 | 0.008116 | 0 | 0.002076 | 0.000151 | 0.003014 | 0.004656 |
| rf23 | 0.001142 | 0.002513 | 0.001407 | 0.005453 | 0 | 0 | 0.003421 | 0.001632 | 0.005475 | 0 |
| rf24 | 0.000854 | 0.005357 | 0 | 0.007991 | 0.001054 | 0.000403 | 0.002633 | 0 | 0.00231 | 0.003788 |
| rf25 | 0 | 0.013547 | 0 | 0.008204 | 0 | 0 | 0.003158 | 0.001131 | 0 | 0.000104 |
| rf26 | 0.001066 | 0.003298 | 0.001039 | 0.000972 | 0.004196 | 0.001709 | 0 | 0.000361 | 1.82E−17 | 0.006138 |
| rf27 | 0.000386 | 0.009366 | 0.006817 | 0.001343 | 0.003125 | 0 | 0.000564 | 0.003643 | 0.002373 | 0.007647 |
| extraTrees0 | 0.000965 | 0.002152 | 0.000476 | 0.001305 | 0.00237 | 0.001551 | 0.001439 | 0.001214 | 0.000529 | 0.003853 |
| extraTrees1 | 0.003307 | 0.004649 | 0.001517 | 0.003485 | 0.001834 | 0.000543 | 0.001867 | 0.003232 | 0.001324 | 0.005698 |
| extraTrees2 | 0.002516 | 0.004927 | 0.00114 | 0.001018 | 0.004125 | 0.001074 | 0.001132 | 0.001054 | 0.001457 | 0.002998 |
| extraTrees3 | 0.000805 | 0.010885 | 0.001229 | 0.000721 | 0.001401 | 0.000466 | 0.000893 | 0.000927 | 0.002392 | 0.000287 |
| extraTrees4 | 0.000696 | 0.002318 | 0.000469 | 0.003265 | 0.004522 | 0 | 0 | 0.001191 | 8.76E−19 | 0.002653 |
| extraTrees5 | 0.002735 | 0.002797 | 0.000672 | 0.002016 | 0.001927 | 0.000451 | 8.76E−19 | 0.001034 | 0.000238 | 0.001951 |
| extraTrees6 | 0.002961 | 0.004962 | 0.000632 | 0.001689 | 0.004518 | 0.001076 | 0.0027 | 0.001439 | 0.000884 | 0.002775 |
| extraTrees7 | 0.001773 | 0.0024 | 0.001321 | 0.000441 | 0.005066 | 0 | 0.000476 | 0.002745 | 3.00E−18 | 0.006779 |
| extraTrees8 | 0.002011 | 0.004975 | 0.000657 | 0.001261 | 0.000386 | 0.000369 | 0.001353 | 0.001649 | 0.000796 | 0.001824 |
| extraTrees9 | 0.00193 | 0.004708 | 0.001194 | 0.00098 | 0.001651 | 2.05E−19 | 0.000987 | 0.00121 | 0.001324 | 0.007583 |
| extraTrees10 | 0.004474 | 0.004959 | 0.000381 | 0.001966 | 0.001723 | 0.001932 | 0.00028 | 0.001012 | 0.001271 | 0.000819 |
| extraTrees11 | 0.004871 | 0.002187 | 0.000786 | 0.001554 | 0.000779 | 0.001055 | 0.00028 | 0.000533 | 0.001662 | 0.00566 |
| extraTrees12 | 0.002602 | 0.006978 | 0.002295 | 0.002939 | 0.005561 | 6.70E−18 | 0.002888 | 0.000145 | 0.000823 | 0.002091 |
| extraTrees13 | 0.003789 | 0.005318 | 0.001195 | 0.001795 | 0.000904 | 0 | 0.001445 | 0.001697 | 0.001996 | 0.001466 |
| extraTrees14 | 0.000965 | 0.002152 | 0.000476 | 0.001305 | 0.00237 | 0.001551 | 0.001439 | 0.001214 | 0.000529 | 0.003853 |
| extraTrees15 | 0.003307 | 0.004649 | 0.001517 | 0.003485 | 0.001834 | 0.000543 | 0.001867 | 0.003232 | 0.001324 | 0.005698 |
| extraTrees16 | 0.002516 | 0.004927 | 0.00114 | 0.001018 | 0.004125 | 0.001074 | 0.001132 | 0.001054 | 0.001457 | 0.002998 |
| extraTrees17 | 0.000805 | 0.010885 | 0.001229 | 0.000721 | 0.001401 | 0.000466 | 0.000893 | 0.000927 | 0.002392 | 0.000287 |
| extraTrees18 | 0.000696 | 0.002318 | 0.000469 | 0.003265 | 0.004522 | 0 | 0 | 0.001191 | 8.76E−19 | 0.002653 |
| extraTrees19 | 0.002735 | 0.002797 | 0.000672 | 0.002016 | 0.001927 | 0.000451 | 8.76E−19 | 0.001034 | 0.000238 | 0.001951 |
| extraTrees20 | 0.002961 | 0.004962 | 0.000632 | 0.001689 | 0.004518 | 0.001076 | 0.0027 | 0.001439 | 0.000884 | 0.002775 |
| extraTrees21 | 0.001773 | 0.0024 | 0.001321 | 0.000441 | 0.005066 | 0 | 0.000476 | 0.002745 | 3.00E−18 | 0.006779 |
| extraTrees22 | 0.002011 | 0.004975 | 0.000657 | 0.001261 | 0.000386 | 0.000369 | 0.001353 | 0.001649 | 0.000796 | 0.001824 |
| extraTrees23 | 0.00193 | 0.004708 | 0.001194 | 0.00098 | 0.001651 | 2.05E−19 | 0.000987 | 0.00121 | 0.001324 | 0.007583 |
| extraTrees24 | 0.004474 | 0.004959 | 0.000381 | 0.001966 | 0.001723 | 0.001932 | 0.00028 | 0.001012 | 0.001271 | 0.000819 |
| extraTrees25 | 0.004871 | 0.002187 | 0.000786 | 0.001554 | 0.000779 | 0.001055 | 0.00028 | 0.000533 | 0.001662 | 0.00566 |
| extraTrees26 | 0.002602 | 0.006978 | 0.002295 | 0.002939 | 0.005561 | 6.70E−18 | 0.002888 | 0.000145 | 0.000823 | 0.002091 |
| extraTrees27 | 0.003789 | 0.005318 | 0.001195 | 0.001795 | 0.000904 | 0 | 0.001445 | 0.001697 | 0.001996 | 0.001466 |
| gbc0 | 0.006896 | 0.000434 | 4.11E−05 | 0.001066 | 1.48E−05 | 0.002014 | 0.000148 | 0 | 8.73E−05 | 0 |
| gbc1 | 0 | 0 | 0 | 0 | 0.006612 | 0 | 0 | 5.41E−05 | 0 | 0.002125 |
| gbc2 | 0.000471 | 0 | 0.034715 | 0 | 0 | 0 | 0 | 0.000267 | 0.005734 | 0.018884 |
| gbc3 | 0 | 0 | 0.000192 | 0 | 0 | 0 | 0.001918 | 3.93E−05 | 0 | 0.000912 |
| gbc4 | 0 | 0.002996 | 0.006245 | 8.78E−05 | 0.000157 | 1.87E−05 | 0 | 0.000291 | 0 | 0.000767 |
| gbc5 | 0 | 0 | 0 | 0 | 4.67E−05 | 0.000715 | 0.002217 | 0 | 0 | 0.017641 |
| gbc6 | 0 | 2.51E−05 | 0.000482 | 0 | 0 | 1.04E−05 | 0.000192 | 0 | 1.59E−05 | 9.29E−05 |
| gbc7 | 0 | 2.69E−05 | 0.001394 | 0 | 0 | 0 | 9.99E−05 | 2.62E−05 | 0 | 9.86E−06 |
| gbc8 | 0 | 3.64E−05 | 0 | 0 | 0 | 0.017665 | 0.003228 | 0 | 0.005837 | 0.007909 |
| gbc9 | 0.000516 | 3.01E−05 | 0 | 0 | 0.004112 | 0.0064 | 0 | 0.006527 | 0.005198 | 0 |
| gbc10 | 0 | 0 | 0 | 0.00027 | 0.010195 | 0.000254 | 0 | 0 | 0 | 0 |
| gbc11 | 4.46E−05 | 1.22E−05 | 0.000539 | 0 | 0 | 0 | 0 | 0 | 0 | 0 |
| gbc12 | 0 | 0.001901 | 0.000117 | 0 | 0.000181 | 0 | 0 | 0 | 0 | 0 |
| gbc13 | 0 | 0.005897 | 2.03E−07 | 0 | 0 | 0 | 0 | 0 | 0.000611 | 0 |
| gbc14 | 0.006896 | 0.000434 | 4.11E−05 | 0.001066 | 1.48E−05 | 0.002014 | 0.000148 | 0 | 8.73E−05 | 0 |
| gbc15 | 0 | 0 | 0 | 0 | 0.006612 | 0 | 0 | 5.41E−05 | 0 | 0.002125 |
| gbc16 | 0.000471 | 0 | 0.034715 | 0 | 0 | 0 | 0 | 0.000267 | 0.005734 | 0.018884 |
| gbc17 | 0 | 0 | 0.000192 | 0 | 0 | 0 | 0.001918 | 3.93E−05 | 0 | 0.000912 |
| gbc18 | 0 | 0.002996 | 0.006245 | 8.78E−05 | 0.000157 | 1.87E−05 | 0 | 0.000291 | 0 | 0.000767 |
| gbc19 | 0 | 0 | 0 | 0 | 4.67E−05 | 0.000715 | 0.002217 | 0 | 0 | 0.017641 |
| gbc20 | 0 | 2.51E−05 | 0.000482 | 0 | 0 | 1.04E−05 | 0.000192 | 0 | 1.59E−05 | 9.29E−05 |
| gbc21 | 0 | 2.69E−05 | 0.001394 | 0 | 0 | 0 | 9.99E−05 | 2.62E−05 | 0 | 9.86E−06 |
| gbc22 | 0 | 3.64E−05 | 0 | 0 | 0 | 0.017665 | 0.003228 | 0 | 0.005837 | 0.007909 |
| gbc23 | 0.000516 | 3.01E−05 | 0 | 0 | 0.004112 | 0.0064 | 0 | 0.006527 | 0.005198 | 0 |
| gbc24 | 0 | 0 | 0 | 0.00027 | 0.010195 | 0.000254 | 0 | 0 | 0 | 0 |
| gbc25 | 4.46E−05 | 1.22E−05 | 0.000539 | 0 | 0 | 0 | 0 | 0 | 0 | 0 |
| gbc26 | 0 | 0.001901 | 0.000117 | 0 | 0.000181 | 0 | 0 | 0 | 0 | 0 |
| gbc27 | 0 | 0.005897 | 2.03E−07 | 0 | 0 | 0 | 0 | 0 | 0.000611 | 0 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | GUSB | GUSBP11 | H3F3B | H3F3C | HAUS1 | HDAC2-AS2 | HEMGN | HIBCH | HINFP | HIST1H3C |
|---|---|---|---|---|---|---|---|---|---|---|
| rf0 | 0.002777 | 4.96E−19 | 0.001245 | 0.000639 | 0 | 0.001528 | 0.000773 | 0 | 0.001115 | 0.000596 |
| rf1 | 0.003349 | 0.002274 | 0 | 0.003808 | 0.000415 | 0.003438 | 0.011161 | 0.002385 | 0 | 0 |
| rf2 | 0.002805 | 0 | 0 | 0.003885 | 0.001712 | 0.001005 | 0.002212 | 0.00283 | 0.001083 | 8.35E−19 |
| rf3 | 0.006029 | 0.008578 | 7.94E−05 | 0.004643 | 0.000255 | 0.001245 | 0.009129 | 0.00528 | 0.000773 | 0.003608 |
| rf4 | 0.00218 | 0.008161 | 0 | 0.004555 | 0.001565 | 6.57E−19 | 0.007035 | 0.00135 | 0 | 5.17E−18 |
| rf5 | 0 | 0.005027 | 0.001962 | 0 | 0.003837 | 0 | 0.000685 | 3.46E−18 | 0.000731 | 0.000696 |
| rf6 | 0.000671 | 0.008214 | 0.00043 | 0.003703 | 0.001707 | 0.001582 | 0.002494 | 0 | 0.001354 | 0.00114 |
| rf7 | 0 | 0.001097 | 0.000329 | 0 | 0.00129 | 0 | 0.003271 | 9.69E−18 | 0.003623 | 0 |
| rf8 | 0.00254 | 0.003089 | 0.000357 | 0 | 0 | 1.41E−18 | 0.001704 | 0.003852 | 0.000768 | 0.002044 |
| rf9 | 0.002224 | 0.003619 | 0 | 0.006589 | 1.04E−17 | 0 | 0.001229 | 0.000999 | 0.002072 | 0.001577 |
| rf10 | 0.003872 | 0.007561 | 0 | 0.002849 | 0 | 0.001657 | 0.005302 | 0.00126 | 4.70E−19 | 0 |
| rf11 | 0.00033 | 0.010143 | 0.002659 | 0.000432 | 0 | 0.003117 | 0.001276 | 0.001319 | 0.001437 | 0.000856 |
| rf12 | 0.000358 | 0.012348 | 0.002047 | 0 | 0.002102 | 0.00118 | 0.00204 | 4.72E−18 | 0.001961 | 0.001458 |
| rf13 | 0.000725 | 0.001483 | 0.000377 | 0.003885 | 0.001326 | 0 | 0.006309 | 0.001886 | 0.004096 | 2.47E−18 |
| rf14 | 0.002777 | 4.96E−19 | 0.001245 | 0.000639 | 0 | 0.001528 | 0.000773 | 0 | 0.001115 | 0.000596 |
| rf15 | 0.003349 | 0.002274 | 0 | 0.003808 | 0.000415 | 0.003438 | 0.011161 | 0.002385 | 0 | 0 |
| rf16 | 0.002805 | 0 | 0 | 0.003885 | 0.001712 | 0.001005 | 0.002212 | 0.00283 | 0.001083 | 8.35E−19 |
| rf17 | 0.006029 | 0.008578 | 7.94E−05 | 0.004643 | 0.000255 | 0.001245 | 0.009129 | 0.00528 | 0.000773 | 0.003608 |
| rf18 | 0.00218 | 0.008161 | 0 | 0.004555 | 0.001565 | 6.57E−19 | 0.007035 | 0.00135 | 0 | 5.17E−18 |
| rf19 | 0 | 0.005027 | 0.001962 | 0 | 0.003837 | 0 | 0.000685 | 3.46E−18 | 0.000731 | 0.000696 |
| rf20 | 0.000671 | 0.008214 | 0.00043 | 0.003703 | 0.001707 | 0.001582 | 0.002494 | 0 | 0.001354 | 0.00114 |
| rf21 | 0 | 0.001097 | 0.000329 | 0 | 0.00129 | 0 | 0.003271 | 9.69E−18 | 0.003623 | 0 |
| rf22 | 0.00254 | 0.003089 | 0.000357 | 0 | 0 | 1.41E−18 | 0.001704 | 0.003852 | 0.000768 | 0.002044 |
| rf23 | 0.002224 | 0.003619 | 0 | 0.006589 | 1.04E−17 | 0 | 0.001229 | 0.000999 | 0.002072 | 0.001577 |
| rf24 | 0.003872 | 0.007561 | 0 | 0.002849 | 0 | 0.001657 | 0.005302 | 0.00126 | 4.70E−19 | 0 |
| rf25 | 0.00033 | 0.010143 | 0.002659 | 0.000432 | 0 | 0.003117 | 0.001276 | 0.001319 | 0.001437 | 0.000856 |
| rf26 | 0.000358 | 0.012348 | 0.002047 | 0 | 0.002102 | 0.00118 | 0.00204 | 4.72E−18 | 0.001961 | 0.001458 |
| rf27 | 0.000725 | 0.001483 | 0.000377 | 0.003885 | 0.001326 | 0 | 0.006309 | 0.001886 | 0.004096 | 2.47E−18 |
| extraTrees0 | 0.002827 | 0.004786 | 0.000737 | 0.001984 | 0.000549 | 0.002283 | 0.002531 | 0.000661 | 0.002457 | 0.001824 |
| extraTrees1 | 0.00049 | 0.003531 | 0.000518 | 0.003307 | 0.005432 | 0.002563 | 0.004521 | 0.000688 | 0.001629 | 0.00146 |
| extraTrees2 | 0.002317 | 0.002793 | 0.000388 | 0.00351 | 0.001927 | 0.002079 | 0.002291 | 0.003742 | 0.001348 | 0.001558 |
| extraTrees3 | 0.00028 | 0.003838 | 0.000749 | 0.001967 | 0.001188 | 0.001381 | 0.001912 | 0.000938 | 0.00324 | 0.001026 |
| extraTrees4 | 0.001121 | 0.002787 | 0.000937 | 0.000568 | 0.001623 | 0.000641 | 0.002035 | 0.001204 | 0.000953 | 0.001275 |
| extraTrees5 | 1.06E−18 | 0.003441 | 0.001586 | 0.002489 | 0.001205 | 0.002526 | 0.002754 | 1.26E−18 | 0.001161 | 0.003155 |
| extraTrees6 | 0.004795 | 0.006669 | 0 | 0.002714 | 0.001052 | 0.0026 | 0.005381 | 0.002765 | 0.00179 | 0.003191 |
| extraTrees7 | 0.002617 | 0.005213 | 0.000667 | 0.001102 | 0.001852 | 0.001872 | 0.001108 | 0.003363 | 0.001918 | 0.003722 |
| extraTrees8 | 0.001408 | 0.0052 | 0.000721 | 0.000888 | 0.000407 | 0.000903 | 0.003218 | 0.004547 | 0.000254 | 0 |
| extraTrees9 | 0.001376 | 0.006559 | 0.000238 | 0.001128 | 0.002737 | 0.000904 | 0.001246 | 0.001563 | 0.003112 | 0.001008 |
| extraTrees10 | 0.000569 | 0.00271 | 0.000775 | 0.001776 | 0.001757 | 0.000568 | 0.006251 | 0.000748 | 0.001321 | 0.003055 |
| extraTrees11 | 0.002327 | 0.001293 | 2.53E−18 | 0.001823 | 0.000209 | 0.002027 | 0.00613 | 7.01E−19 | 0.00188 | 0.002284 |
| extraTrees12 | 0.001007 | 0.002839 | 0.000975 | 0.001868 | 0.003841 | 0.001744 | 0.003309 | 0.000767 | 0.003955 | 0.001687 |
| extraTrees13 | 0.001099 | 0.001674 | 0.000774 | 0.002104 | 0.00354 | 0.001492 | 0.005548 | 0.000491 | 0.001378 | 0 |
| extraTrees14 | 0.002827 | 0.004786 | 0.000737 | 0.001984 | 0.000549 | 0.002283 | 0.002531 | 0.000661 | 0.002457 | 0.001824 |
| extraTrees15 | 0.00049 | 0.003531 | 0.000518 | 0.003307 | 0.005432 | 0.002563 | 0.004521 | 0.000688 | 0.001629 | 0.00146 |
| extraTrees16 | 0.002317 | 0.002793 | 0.000388 | 0.00351 | 0.001927 | 0.002079 | 0.002291 | 0.003742 | 0.001348 | 0.001558 |
| extraTrees17 | 0.00028 | 0.003838 | 0.000749 | 0.001967 | 0.001188 | 0.001381 | 0.001912 | 0.000938 | 0.00324 | 0.001026 |
| extraTrees18 | 0.001121 | 0.002787 | 0.000937 | 0.000568 | 0.001623 | 0.000641 | 0.002035 | 0.001204 | 0.000953 | 0.001275 |
| extraTrees19 | 1.06E−18 | 0.003441 | 0.001586 | 0.002489 | 0.001205 | 0.002526 | 0.002754 | 1.26E−18 | 0.001161 | 0.003155 |
| extraTrees20 | 0.004795 | 0.006669 | 0 | 0.002714 | 0.001052 | 0.0026 | 0.005381 | 0.002765 | 0.00179 | 0.003191 |
| extraTrees21 | 0.002617 | 0.005213 | 0.000667 | 0.001102 | 0.001852 | 0.001872 | 0.001108 | 0.003363 | 0.001918 | 0.003722 |
| extraTrees22 | 0.001408 | 0.0052 | 0.000721 | 0.000888 | 0.000407 | 0.000903 | 0.003218 | 0.004547 | 0.000254 | 0 |
| extraTrees23 | 0.001376 | 0.006559 | 0.000238 | 0.001128 | 0.002737 | 0.000904 | 0.001246 | 0.001563 | 0.003112 | 0.001008 |
| extraTrees24 | 0.000569 | 0.00271 | 0.000775 | 0.001776 | 0.001757 | 0.000568 | 0.006251 | 0.000748 | 0.001321 | 0.003055 |
| extraTrees25 | 0.002327 | 0.001293 | 2.53E−18 | 0.001823 | 0.000209 | 0.002027 | 0.00613 | 7.01E−19 | 0.00188 | 0.002284 |
| extraTrees26 | 0.001007 | 0.002839 | 0.000975 | 0.001868 | 0.003841 | 0.001744 | 0.003309 | 0.000767 | 0.003955 | 0.001687 |
| extraTrees27 | 0.001099 | 0.001674 | 0.000774 | 0.002104 | 0.00354 | 0.001492 | 0.005548 | 0.000491 | 0.001378 | 0 |
| gbc0 | 0 | 9.23E−05 | 0 | 0 | 0 | 0 | 8.62E−05 | 0.000104 | 0 | 0.006447 |
| gbc1 | 0.000151 | 0.003058 | 0 | 0 | 0 | 0 | 0.028092 | 9.95E−05 | 5.93E−05 | 0 |
| gbc2 | 0.004852 | 0.02146 | 3.78E−05 | 0 | 0 | 2.39E−05 | 0 | 0 | 0 | 0 |
| gbc3 | 0 | 0.000244 | 0 | 0.001088 | 0.000206 | 0 | 0.029714 | 0 | 0 | 0 |
| gbc4 | 0 | 0.011902 | 0.000179 | 0 | 0.000794 | 0 | 0 | 0.012927 | 0 | 0.00019 |
| gbc5 | 0.006211 | 0.022779 | 0 | 1.87E−06 | 0 | 0 | 0 | 0.003064 | 3.32E−05 | 0 |
| gbc6 | 0 | 0.013912 | 0 | 2.20E−05 | 0 | 0 | 0.000341 | 0 | 0.007322 | 0 |
| gbc7 | 0.00019 | 0.000814 | 0 | 0 | 0.002803 | 0 | 0.000989 | 0 | 0 | 0 |
| gbc8 | 0.000166 | 0.048668 | 6.53E−05 | 3.13E−07 | 3.75E−05 | 0.000248 | 0 | 0 | 0 | 0 |
| gbc9 | 0 | 8.55E−05 | 0 | 4.58E−06 | 6.23E−05 | 0 | 0 | 0 | 0.000642 | 9.55E−05 |
| gbc10 | 0.004294 | 0.000805 | 0 | 0 | 0.002795 | 0.000587 | 6.86E−06 | 0 | 0 | 0 |
| gbc11 | 0 | 0.005711 | 0 | 0.004027 | 0.002356 | 0 | 0.002912 | 9.14E−05 | 0 | 9.40E−05 |
| gbc12 | 8.87E−05 | 0.000259 | 0 | 0 | 0 | 0 | 0 | 0 | 0 | 7.22E−07 |
| gbc13 | 0 | 0.031637 | 0 | 0 | 4.68E−07 | 0 | 0.00026 | 0 | 0 | 0 |
| gbc14 | 0 | 9.23E−05 | 0 | 0 | 0 | 0 | 8.62E−05 | 0.000104 | 0 | 0.006447 |
| gbc15 | 0.000151 | 0.003058 | 0 | 0 | 0 | 0 | 0.028092 | 9.95E−05 | 5.93E−05 | 0 |
| gbc16 | 0.004852 | 0.02146 | 3.78E−05 | 0 | 0 | 2.39E−05 | 0 | 0 | 0 | 0 |
| gbc17 | 0 | 0.000244 | 0 | 0.001088 | 0.000206 | 0 | 0.029714 | 0 | 0 | 0 |
| gbc18 | 0 | 0.011902 | 0.000179 | 0 | 0.000794 | 0 | 0 | 0.012927 | 0 | 0.00019 |
| gbc19 | 0.006211 | 0.022779 | 0 | 1.87E−06 | 0 | 0 | 0 | 0.003064 | 3.32E−05 | 0 |
| gbc20 | 0 | 0.013912 | 0 | 2.20E−05 | 0 | 0 | 0.000341 | 0 | 0.007322 | 0 |
| gbc21 | 0.00019 | 0.000814 | 0 | 0 | 0.002803 | 0 | 0.000989 | 0 | 0 | 0 |
| gbc22 | 0.000166 | 0.048668 | 6.53E−05 | 3.13E−07 | 3.75E−05 | 0.000248 | 0 | 0 | 0 | 0 |
| gbc23 | 0 | 8.55E−05 | 0 | 4.58E−06 | 6.23E−05 | 0 | 0 | 0 | 0.000642 | 9.55E−05 |
| gbc24 | 0.004294 | 0.000805 | 0 | 0 | 0.002795 | 0.000587 | 6.86E−06 | 0 | 0 | 0 |
| gbc25 | 0 | 0.005711 | 0 | 0.004027 | 0.002356 | 0 | 0.002912 | 9.14E−05 | 0 | 9.40E−05 |
| gbc26 | 8.87E−05 | 0.000259 | 0 | 0 | 0 | 0 | 0 | 0 | 0 | 7.22E−07 |
| gbc27 | 0 | 0.031637 | 0 | 0 | 4.68E−07 | 0 | 0.00026 | 0 | 0 | 0 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | HLA-G | HLA-J | IDE | IER3IP1 | IFT57 | IFT74 | IGF2BP2 | IL7 | IMPA1 | IMPACT |
|---|---|---|---|---|---|---|---|---|---|---|
| rf0 | 0.003734 | 0.000173 | 0.002434 | 0 | 0.001121 | 0 | 3.23E−18 | 0.001389 | 2.66E−18 | 0.005324 |
| rf1 | 0.004086 | 0.000777 | 0.008464 | 0 | 0.003881 | 0.000464 | 0 | 0 | 0 | 0.00127 |
| rf2 | 0.001463 | 0.000321 | 0.002636 | 0.000908 | 0 | 0.002851 | 0.000974 | 0.00069 | 0 | 0.005108 |
| rf3 | 0 | 0.00558 | 0.002296 | 0.001235 | 0 | 0.001419 | 8.68E−18 | 0 | 0 | 0.005819 |
| rf4 | 0.001175 | 0.000785 | 4.39E−18 | 0.001024 | 0.000878 | 0.001549 | 0.003699 | 0 | 0 | 0.002666 |
| rf5 | 0.000954 | 0.00167 | 0.002911 | 0.00097 | 0.000384 | 0 | 0.000813 | 0.000366 | 0.000339 | 0.000625 |
| rf6 | 0 | 0 | 0.003948 | 0 | 0.002623 | 0.000285 | 7.81E−05 | 0 | 0.002933 | 0.006198 |
| rf7 | 0 | 0 | 2.96E−05 | 0 | 0 | 0.000345 | 0.00181 | 4.38E−19 | 0 | 0 |
| rf8 | 0.002569 | 0.000965 | 0 | 0.002234 | 0.003262 | 0.000978 | 0.000824 | 0.000304 | 0.00364 | 0.000661 |
| rf9 | 0.005409 | 0.003384 | 0 | 0.000797 | 0.00435 | 0.000346 | 0.002771 | 4.85E−19 | 0.001098 | 0.003701 |
| rf10 | 0.001871 | 0.001253 | 0.000968 | 0.002766 | 0.001979 | 0.007621 | 4.38E−18 | 0 | 0.001829 | 0.006068 |
| rf11 | 0.001526 | 0.005742 | 0 | 0.001116 | 0.002191 | 0.002372 | 0 | 0.003165 | 0.001449 | 0.001561 |
| rf12 | 0.002312 | 0.001939 | 0.001208 | 0.002711 | 0.007863 | 0 | 0.00302 | 0.003408 | 0 | 0.001271 |
| rf13 | 0.001747 | 0.008588 | 0.00287 | 0.000684 | 0.015849 | 0.001231 | 0.001063 | 0 | 0 | 0.002816 |
| rf14 | 0.003734 | 0.000173 | 0.002434 | 0 | 0.001121 | 0 | 3.23E−18 | 0.001389 | 2.66E−18 | 0.005324 |
| rf15 | 0.004086 | 0.000777 | 0.008464 | 0 | 0.003881 | 0.000464 | 0 | 0 | 0 | 0.00127 |
| rf16 | 0.001463 | 0.000321 | 0.002636 | 0.000908 | 0 | 0.002851 | 0.000974 | 0.00069 | 0 | 0.005108 |
| rf17 | 0 | 0.00558 | 0.002296 | 0.001235 | 0 | 0.001419 | 8.68E−18 | 0 | 0 | 0.005819 |
| rf18 | 0.001175 | 0.000785 | 4.39E−18 | 0.001024 | 0.000878 | 0.001549 | 0.003699 | 0 | 0 | 0.002666 |
| rf19 | 0.000954 | 0.00167 | 0.002911 | 0.00097 | 0.000384 | 0 | 0.000813 | 0.000366 | 0.000339 | 0.000625 |
| rf20 | 0 | 0 | 0.003948 | 0 | 0.002623 | 0.000285 | 7.81E−05 | 0 | 0.002933 | 0.006198 |
| rf21 | 0 | 0 | 2.96E−05 | 0 | 0 | 0.000345 | 0.00181 | 4.38E−19 | 0 | 0 |
| rf22 | 0.002569 | 0.000965 | 0 | 0.002234 | 0.003262 | 0.000978 | 0.000824 | 0.000304 | 0.00364 | 0.000661 |
| rf23 | 0.005409 | 0.003384 | 0 | 0.000797 | 0.00435 | 0.000346 | 0.002771 | 4.85E−19 | 0.001098 | 0.003701 |
| rf24 | 0.001871 | 0.001253 | 0.000968 | 0.002766 | 0.001979 | 0.007621 | 4.38E−18 | 0 | 0.001829 | 0.006068 |
| rf25 | 0.001526 | 0.005742 | 0 | 0.001116 | 0.002191 | 0.002372 | 0 | 0.003165 | 0.001449 | 0.001561 |
| rf26 | 0.002312 | 0.001939 | 0.001208 | 0.002711 | 0.007863 | 0 | 0.00302 | 0.003408 | 0 | 0.001271 |
| rf27 | 0.001747 | 0.008588 | 0.00287 | 0.000684 | 0.015849 | 0.001231 | 0.001063 | 0 | 0 | 0.002816 |
| extraTrees0 | 1.14E−17 | 0.001404 | 0.000511 | 4.31E−05 | 0.001326 | 0.001048 | 0.000908 | 0.000772 | 0.000858 | 0.004454 |
| extraTrees1 | 9.22E−19 | 0.00162 | 0.001567 | 0.001414 | 0.002686 | 5.26E−19 | 0.000701 | 0.002197 | 0.000778 | 0.000705 |
| extraTrees2 | 0.001428 | 0.000959 | 0.002192 | 0.001128 | 0.000999 | 0.002705 | 0.001787 | 7.00E−19 | 0.00189 | 0.00354 |
| extraTrees3 | 0.001583 | 0 | 0.000567 | 0.001437 | 0.004063 | 0.002922 | 0.000634 | 0.000788 | 0.002195 | 0 |
| extraTrees4 | 0.002847 | 0.000239 | 0.000989 | 0.000599 | 0.001671 | 0.00088 | 0.00188 | 0.001374 | 0.000388 | 0.000388 |
| extraTrees5 | 5.53E−05 | 0.001461 | 0.001863 | 0.00116 | 0.002229 | 0.000926 | 0 | 6.20E−18 | 0.002034 | 0.006795 |
| extraTrees6 | 0.001329 | 5.29E−05 | 0.003729 | 0.001255 | 0.002612 | 0.00026 | 0.001156 | 0.000248 | 0.001076 | 0.000746 |
| extraTrees7 | 0.003181 | 0.001241 | 0.002247 | 0.001029 | 0.003677 | 0.000737 | 0.001076 | 0 | 0.00185 | 0.001515 |
| extraTrees8 | 0.000417 | 0.001534 | 0.001211 | 0.00067 | 0.004984 | 0.000819 | 0.001132 | 0.000288 | 0.002931 | 0.003171 |
| extraTrees9 | 0.0022 | 0.00095 | 0.000476 | 0 | 0.001312 | 0.000275 | 0.000727 | 0 | 0.000848 | 0.002619 |
| extraTrees10 | 0.000832 | 0.000388 | 0.001649 | 0 | 0.000812 | 0.001234 | 0.000473 | 0.000945 | 0.000821 | 0.000587 |
| extraTrees11 | 0.00136 | 0.001206 | 0.002538 | 0.000793 | 0.001269 | 0.000492 | 0.001407 | 0.000713 | 0.000348 | 0.001373 |
| extraTrees12 | 0.001527 | 0.000885 | 0.001892 | 0.001555 | 0.002138 | 0.000268 | 0.00397 | 0.000582 | 0.000862 | 0.000469 |
| extraTrees13 | 0.002269 | 0.000268 | 0.00322 | 0.001696 | 0.002353 | 0.000947 | 0.000492 | 0.001255 | 0.002363 | 0.002486 |
| extraTrees14 | 1.14E−17 | 0.001404 | 0.000511 | 4.31E−05 | 0.001326 | 0.001048 | 0.000908 | 0.000772 | 0.000858 | 0.004454 |
| extraTrees15 | 9.22E−19 | 0.00162 | 0.001567 | 0.001414 | 0.002686 | 5.26E−19 | 0.000701 | 0.002197 | 0.000778 | 0.000705 |
| extraTrees16 | 0.001428 | 0.000959 | 0.002192 | 0.001128 | 0.000999 | 0.002705 | 0.001787 | 7.00E−19 | 0.00189 | 0.00354 |
| extraTrees17 | 0.001583 | 0 | 0.000567 | 0.001437 | 0.004063 | 0.002922 | 0.000634 | 0.000788 | 0.002195 | 0 |
| extraTrees18 | 0.002847 | 0.000239 | 0.000989 | 0.000599 | 0.001671 | 0.00088 | 0.00188 | 0.001374 | 0.000388 | 0.000388 |
| extraTrees19 | 5.53E−05 | 0.001461 | 0.001863 | 0.00116 | 0.002229 | 0.000926 | 0 | 6.20E−18 | 0.002034 | 0.006795 |
| extraTrees20 | 0.001329 | 5.29E−05 | 0.003729 | 0.001255 | 0.002612 | 0.00026 | 0.001156 | 0.000248 | 0.001076 | 0.000746 |
| extraTrees21 | 0.003181 | 0.001241 | 0.002247 | 0.001029 | 0.003677 | 0.000737 | 0.001076 | 0 | 0.00185 | 0.001515 |
| extraTrees22 | 0.000417 | 0.001534 | 0.001211 | 0.00067 | 0.004984 | 0.000819 | 0.001132 | 0.000288 | 0.002931 | 0.003171 |
| extraTrees23 | 0.0022 | 0.00095 | 0.000476 | 0 | 0.001312 | 0.000275 | 0.000727 | 0 | 0.000848 | 0.002619 |
| extraTrees24 | 0.000832 | 0.000388 | 0.001649 | 0 | 0.000812 | 0.001234 | 0.000473 | 0.000945 | 0.000821 | 0.000587 |
| extraTrees25 | 0.00136 | 0.001206 | 0.002538 | 0.000793 | 0.001269 | 0.000492 | 0.001407 | 0.000713 | 0.000348 | 0.001373 |
| extraTrees26 | 0.001527 | 0.000885 | 0.001892 | 0.001555 | 0.002138 | 0.000268 | 0.00397 | 0.000582 | 0.000862 | 0.000469 |
| extraTrees27 | 0.002269 | 0.000268 | 0.00322 | 0.001696 | 0.002353 | 0.000947 | 0.000492 | 0.001255 | 0.002363 | 0.002486 |
| gbc0 | 1.46E−05 | 3.09E−05 | 0 | 0 | 0.003442 | 0 | 0 | 0 | 1.51E−05 | 0.006388 |
| gbc1 | 9.08E−05 | 0 | 0 | 0 | 0.005182 | 5.61E−07 | 9.94E−06 | 0 | 0 | 0.002903 |
| gbc2 | 0 | 0.00439 | 0 | 0 | 0 | 0 | 0 | 0.003799 | 0 | 0.014923 |
| gbc3 | 0.003765 | 4.04E−05 | 0.000159 | 0 | 1.37E−05 | 0.0024 | 0 | 0 | 0.000572 | 0.013751 |
| gbc4 | 0 | 7.68E−05 | 0 | 0 | 0 | 0 | 7.63E−05 | 0 | 0 | 3.05E−05 |
| gbc5 | 0 | 3.82E−05 | 0 | 0 | 3.22E−05 | 0 | 0 | 0.000374 | 0.018679 | 0 |
| gbc6 | 0 | 0 | 0.000388 | 0.000144 | 0.000776 | 0.000945 | 0 | 0 | 3.48E−05 | 0 |
| gbc7 | 2.22E−05 | 1.83E−07 | 0 | 0.006377 | 0.023086 | 0.001286 | 4.36E−05 | 0 | 0.005903 | 0.001135 |
| gbc8 | 0.02054 | 0 | 0.001541 | 0 | 0 | 0 | 0.000139 | 8.67E−05 | 3.12E−05 | 3.05E−05 |
| gbc9 | 0 | 0 | 0 | 0.001157 | 9.88E−06 | 0 | 2.61E−06 | 0.000766 | 0 | 0.003321 |
| gbc10 | 0 | 0.000646 | 0 | 0 | 0.0175 | 0 | 0 | 0 | 0.001131 | 0.023869 |
| gbc11 | 0 | 0.001231 | 0 | 0 | 0.006384 | 0.00074 | 0 | 0.00568 | 0 | 0 |
| gbc12 | 0.007474 | 0 | 0.00926 | 0.0007 | 0 | 0.004203 | 0 | 1.60E−05 | 5.02E−05 | 0.002376 |
| gbc13 | 0 | 0.006401 | 0 | 0 | 0.000871 | 0 | 6.94E−05 | 0.000499 | 0 | 0.001175 |
| gbc14 | 1.46E−05 | 3.09E−05 | 0 | 0 | 0.003442 | 0 | 0 | 0 | 1.51E−05 | 0.006388 |
| gbc15 | 9.08E−05 | 0 | 0 | 0 | 0.005182 | 5.61E−07 | 9.94E−06 | 0 | 0 | 0.002903 |
| gbc16 | 0 | 0.00439 | 0 | 0 | 0 | 0 | 0 | 0.003799 | 0 | 0.014923 |
| gbc17 | 0.003765 | 4.04E−05 | 0.000159 | 0 | 1.37E−05 | 0.0024 | 0 | 0 | 0.000572 | 0.013751 |
| gbc18 | 0 | 7.68E−05 | 0 | 0 | 0 | 0 | 7.63E−05 | 0 | 0 | 3.05E−05 |
| gbc19 | 0 | 3.82E−05 | 0 | 0 | 3.22E−05 | 0 | 0 | 0.000374 | 0.018679 | 0 |
| gbc20 | 0 | 0 | 0.000388 | 0.000144 | 0.000776 | 0.000945 | 0 | 0 | 3.48E−05 | 0 |
| gbc21 | 2.22E−05 | 1.83E−07 | 0 | 0.006377 | 0.023086 | 0.001286 | 4.36E−05 | 0 | 0.005903 | 0.001135 |
| gbc22 | 0.02054 | 0 | 0.001541 | 0 | 0 | 0 | 0.000139 | 8.67E−05 | 3.12E−05 | 3.05E−05 |
| gbc23 | 0 | 0 | 0 | 0.001157 | 9.88E−06 | 0 | 2.61E−06 | 0.000766 | 0 | 0.003321 |
| gbc24 | 0 | 0.000646 | 0 | 0 | 0.0175 | 0 | 0 | 0 | 0.001131 | 0.023869 |
| gbc25 | 0 | 0.001231 | 0 | 0 | 0.006384 | 0.00074 | 0 | 0.00568 | 0 | 0 |
| gbc26 | 0.007474 | 0 | 0.00926 | 0.0007 | 0 | 0.004203 | 0 | 1.60E−05 | 5.02E−05 | 0.002376 |
| gbc27 | 0 | 0.006401 | 0 | 0 | 0.000871 | 0 | 6.94E−05 | 0.000499 | 0 | 0.001175 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | ING4 | INSIG2 | IQCB1 | ITGB5 | JRKL | JTB | KBTBD2 | KDM3A | KHDRBS2 | KHK |
|---|---|---|---|---|---|---|---|---|---|---|
| rf0 | 0 | 0.00059 | 0.003157 | 0.00181 | 0.001317 | 5.59E−18 | 0.000357 | 0.014262 | 0.000313 | 0 |
| rf1 | 0 | 0 | 0.002833 | 0.000818 | 0.003975 | 0.003608 | 0 | 0 | 0.008516 | 0.000377 |
| rf2 | 0 | 0.00036 | 7.51E−05 | 0 | 0 | 0 | 1.49E−18 | 0.013331 | 0.000919 | 0.001889 |
| rf3 | 0.000298 | 0 | 0 | 0.003055 | 0 | 0.008591 | 0 | 0.009154 | 0 | 0.001549 |
| rf4 | 1.12E−17 | 0 | 0 | 2.44E−18 | 1.56E−18 | 0.000386 | 5.35E−19 | 0.00427 | 0 | 0 |
| rf5 | 0 | 0.000417 | 9.15E−19 | 0.001168 | 0 | 0.006956 | 0.000359 | 0.002526 | 0 | 0.000569 |
| rf6 | 0.0043 | 0.002146 | 0.006967 | 0 | 0.001087 | 0.000358 | 0.003035 | 0.011185 | 0.001916 | 0.000359 |
| rf7 | 0.00058 | 0 | 0.005316 | 0.002994 | 0.005899 | 0 | 0 | 0.003106 | 0.002253 | 9.30E−19 |
| rf8 | 0 | 0.001593 | 0 | 0.002015 | 0.003559 | 0.000394 | 0 | 0.00336 | 0.002521 | 0.000366 |
| rf9 | 0 | 0 | 0 | 4.70E−18 | 0.000691 | 0 | 0.001164 | 0.00568 | 0.001992 | 0.000358 |
| rf10 | 0.000417 | 0 | 0.004767 | 7.26E−19 | 0.003029 | 0.001342 | 0.001299 | 0.007146 | 0.008356 | 0.002593 |
| rf11 | 0 | 0 | 0.003884 | 0.002773 | 0 | 0.007113 | 0.002906 | 0 | 0 | 0.001292 |
| rf12 | 0.001106 | 0.002245 | 0.000281 | 0.000604 | 0.001693 | 0.007521 | 5.21E−19 | 0.005256 | 0.000671 | 0.002376 |
| rf13 | 0 | 0 | 0 | 0 | 0.001094 | 0.000938 | 0.00065 | 0.007592 | 1.09E−18 | 0 |
| rf14 | 0 | 0.00059 | 0.003157 | 0.00181 | 0.001317 | 5.59E−18 | 0.000357 | 0.014262 | 0.000313 | 0 |
| rf15 | 0 | 0 | 0.002833 | 0.000818 | 0.003975 | 0.003608 | 0 | 0 | 0.008516 | 0.000377 |
| rf16 | 0 | 0.00036 | 7.51E−05 | 0 | 0 | 0 | 1.49E−18 | 0.013331 | 0.000919 | 0.001889 |
| rf17 | 0.000298 | 0 | 0 | 0.003055 | 0 | 0.008591 | 0 | 0.009154 | 0 | 0.001549 |
| rf18 | 1.12E−17 | 0 | 0 | 2.44E−18 | 1.56E−18 | 0.000386 | 5.35E−19 | 0.00427 | 0 | 0 |
| rf19 | 0 | 0.000417 | 9.15E−19 | 0.001168 | 0 | 0.006956 | 0.000359 | 0.002526 | 0 | 0.000569 |
| rf20 | 0.0043 | 0.002146 | 0.006967 | 0 | 0.001087 | 0.000358 | 0.003035 | 0.011185 | 0.001916 | 0.000359 |
| rf21 | 0.00058 | 0 | 0.005316 | 0.002994 | 0.005899 | 0 | 0 | 0.003106 | 0.002253 | 9.30E−19 |
| rf22 | 0 | 0.001593 | 0 | 0.002015 | 0.003559 | 0.000394 | 0 | 0.00336 | 0.002521 | 0.000366 |
| rf23 | 0 | 0 | 0 | 4.70E−18 | 0.000691 | 0 | 0.001164 | 0.00568 | 0.001992 | 0.000358 |
| rf24 | 0.000417 | 0 | 0.004767 | 7.26E−19 | 0.003029 | 0.001342 | 0.001299 | 0.007146 | 0.008356 | 0.002593 |
| rf25 | 0 | 0 | 0.003884 | 0.002773 | 0 | 0.007113 | 0.002906 | 0 | 0 | 0.001292 |
| rf26 | 0.001106 | 0.002245 | 0.000281 | 0.000604 | 0.001693 | 0.007521 | 5.21E−19 | 0.005256 | 0.000671 | 0.002376 |
| rf27 | 0 | 0 | 0 | 0 | 0.001094 | 0.000938 | 0.00065 | 0.007592 | 1.09E−18 | 0 |
| extraTrees0 | 0.000976 | 0.001365 | 0.000969 | 0.000994 | 0.003067 | 0.0021 | 0.001955 | 0.005441 | 0.001927 | 0.002618 |
| extraTrees1 | 0.001073 | 0.000736 | 0.003362 | 0.000762 | 0.001106 | 0.002624 | 0.000235 | 0.004697 | 0.002727 | 0.000373 |
| extraTrees2 | 0.000208 | 0.001154 | 0.002692 | 0.000625 | 0.000422 | 0.001437 | 0.001245 | 0.008799 | 0.005524 | 0.001655 |
| extraTrees3 | 0.000947 | 0.000796 | 0.000397 | 0.000739 | 0.002083 | 0.004427 | 0.000507 | 0.003573 | 0.001445 | 0.000677 |
| extraTrees4 | 0.000344 | 0.000238 | 0.002156 | 0.002549 | 0.000238 | 0.000495 | 0.003129 | 0.007117 | 0.002253 | 0.002549 |
| extraTrees5 | 0 | 0.000921 | 0.00428 | 0.000713 | 0.00028 | 0.005347 | 0.000518 | 0.006475 | 0.003106 | 0.001359 |
| extraTrees6 | 0.001052 | 0.000905 | 0.002141 | 0.001036 | 0.005933 | 0.003233 | 8.54E−19 | 0.007399 | 0.001249 | 0.000297 |
| extraTrees7 | 0.000642 | 0.000388 | 0.001794 | 0.002254 | 0.001619 | 0.001438 | 0.00062 | 0.005402 | 5.99E−05 | 0.001126 |
| extraTrees8 | 0.000258 | 0.001048 | 0.000905 | 0.002739 | 0.00125 | 0.006965 | 0.000268 | 0.004553 | 0.000668 | 0.001921 |
| extraTrees9 | 0.002528 | 0 | 0.000576 | 0.00043 | 0.002561 | 0.002184 | 0.001103 | 0.002117 | 0 | 0.001808 |
| extraTrees10 | 0.001585 | 2.87E−18 | 0.003836 | 0.000751 | 0.000998 | 0.000652 | 5.73E−05 | 0.007624 | 0.001427 | 0.001905 |
| extraTrees11 | 6.15E−19 | 0.000238 | 0.000295 | 0.000845 | 0.001673 | 0.004476 | 0.000921 | 0.007167 | 0.000801 | 0.000903 |
| extraTrees12 | 2.05E−19 | 2.10E−05 | 0.001304 | 0.002157 | 0.001251 | 0.002639 | 0.000959 | 0.013915 | 0.00065 | 0.000837 |
| extraTrees13 | 0.000843 | 0.001847 | 0.002945 | 0.002751 | 0.000555 | 0.003774 | 0.002845 | 0.008843 | 0.000879 | 0.00067 |
| extraTrees14 | 0.000976 | 0.001365 | 0.000969 | 0.000994 | 0.003067 | 0.0021 | 0.001955 | 0.005441 | 0.001927 | 0.002618 |
| extraTrees15 | 0.001073 | 0.000736 | 0.003362 | 0.000762 | 0.001106 | 0.002624 | 0.000235 | 0.004697 | 0.002727 | 0.000373 |
| extraTrees16 | 0.000208 | 0.001154 | 0.002692 | 0.000625 | 0.000422 | 0.001437 | 0.001245 | 0.008799 | 0.005524 | 0.001655 |
| extraTrees17 | 0.000947 | 0.000796 | 0.000397 | 0.000739 | 0.002083 | 0.004427 | 0.000507 | 0.003573 | 0.001445 | 0.000677 |
| extraTrees18 | 0.000344 | 0.000238 | 0.002156 | 0.002549 | 0.000238 | 0.000495 | 0.003129 | 0.007117 | 0.002253 | 0.002549 |
| extraTrees19 | 0 | 0.000921 | 0.00428 | 0.000713 | 0.00028 | 0.005347 | 0.000518 | 0.006475 | 0.003106 | 0.001359 |
| extraTrees20 | 0.001052 | 0.000905 | 0.002141 | 0.001036 | 0.005933 | 0.003233 | 8.54E−19 | 0.007399 | 0.001249 | 0.000297 |
| extraTrees21 | 0.000642 | 0.000388 | 0.001794 | 0.002254 | 0.001619 | 0.001438 | 0.00062 | 0.005402 | 5.99E−05 | 0.001126 |
| extraTrees22 | 0.000258 | 0.001048 | 0.000905 | 0.002739 | 0.00125 | 0.006965 | 0.000268 | 0.004553 | 0.000668 | 0.001921 |
| extraTrees23 | 0.002528 | 0 | 0.000576 | 0.00043 | 0.002561 | 0.002184 | 0.001103 | 0.002117 | 0 | 0.001808 |
| extraTrees24 | 0.001585 | 2.87E−18 | 0.003836 | 0.000751 | 0.000998 | 0.000652 | 5.73E−05 | 0.007624 | 0.001427 | 0.001905 |
| extraTrees25 | 6.15E−19 | 0.000238 | 0.000295 | 0.000845 | 0.001673 | 0.004476 | 0.000921 | 0.007167 | 0.000801 | 0.000903 |
| extraTrees26 | 2.05E−19 | 2.10E−05 | 0.001304 | 0.002157 | 0.001251 | 0.002639 | 0.000959 | 0.013915 | 0.00065 | 0.000837 |
| extraTrees27 | 0.000843 | 0.001847 | 0.002945 | 0.002751 | 0.000555 | 0.003774 | 0.002845 | 0.008843 | 0.000879 | 0.00067 |
| gbc0 | 1.77E−05 | 0 | 0 | 0.00014 | 8.06E−05 | 0.000143 | 0 | 0.042012 | 0 | 0 |
| gbc1 | 0 | 0 | 0.003163 | 0 | 0.000684 | 0.000104 | 0 | 0.000845 | 0 | 0.001991 |
| gbc2 | 0.00028 | 3.50E−05 | 0.005084 | 0 | 0.000122 | 0.006657 | 0 | 0.035481 | 0.007523 | 0 |
| gbc3 | 0 | 7.45E−06 | 0 | 4.78E−05 | 0.000204 | 0.030928 | 0 | 0.004267 | 0.000114 | 6.54E−06 |
| gbc4 | 0 | 0 | 0.001201 | 0.008398 | 4.27E−05 | 0.014618 | 0.000192 | 0 | 0 | 0 |
| gbc5 | 0 | 0.000265 | 0.016531 | 0 | 0 | 0.000122 | 0.000761 | 0.003873 | 0 | 0 |
| gbc6 | 0 | 0.004404 | 6.74E−05 | 0 | 7.77E−05 | 0.000236 | 0 | 0.021162 | 1.44E−05 | 0 |
| gbc7 | 0.003104 | 0 | 0.000363 | 0.005144 | 5.88E−05 | 0.046558 | 0 | 0.000268 | 0.008167 | 0 |
| gbc8 | 0 | 0.001524 | 9.17E−05 | 4.25E−07 | 0.001152 | 0 | 0 | 0.005964 | 0 | 0 |
| gbc9 | 0 | 8.96E−07 | 0.000237 | 0.003111 | 0 | 0 | 0.000124 | 0.032515 | 0 | 0 |
| gbc10 | 0 | 0 | 3.03E−05 | 0 | 0.000209 | 0 | 0.003624 | 0.001704 | 0 | 0 |
| gbc11 | 0 | 0 | 0 | 0 | 0.000484 | 0 | 0 | 0 | 0 | 0 |
| gbc12 | 0 | 0.00018 | 0.003059 | 0.000101 | 0 | 0.008272 | 0 | 0.000246 | 0 | 0 |
| gbc13 | 0 | 0 | 9.47E−05 | 0 | 0 | 0 | 0 | 0.000937 | 0 | 0 |
| gbc14 | 1.77E−05 | 0 | 0 | 0.00014 | 8.06E−05 | 0.000143 | 0 | 0.042012 | 0 | 0 |
| gbc15 | 0 | 0 | 0.003163 | 0 | 0.000684 | 0.000104 | 0 | 0.000845 | 0 | 0.001991 |
| gbc16 | 0.00028 | 3.50E−05 | 0.005084 | 0 | 0.000122 | 0.006657 | 0 | 0.035481 | 0.007523 | 0 |
| gbc17 | 0 | 7.45E−06 | 0 | 4.78E−05 | 0.000204 | 0.030928 | 0 | 0.004267 | 0.000114 | 6.54E−06 |
| gbc18 | 0 | 0 | 0.001201 | 0.008398 | 4.27E−05 | 0.014618 | 0.000192 | 0 | 0 | 0 |
| gbc19 | 0 | 0.000265 | 0.016531 | 0 | 0 | 0.000122 | 0.000761 | 0.003873 | 0 | 0 |
| gbc20 | 0 | 0.004404 | 6.74E−05 | 0 | 7.77E−05 | 0.000236 | 0 | 0.021162 | 1.44E−05 | 0 |
| gbc21 | 0.003104 | 0 | 0.000363 | 0.005144 | 5.88E−05 | 0.046558 | 0 | 0.000268 | 0.008167 | 0 |
| gbc22 | 0 | 0.001524 | 9.17E−05 | 4.25E−07 | 0.001152 | 0 | 0 | 0.005964 | 0 | 0 |
| gbc23 | 0 | 8.96E−07 | 0.000237 | 0.003111 | 0 | 0 | 0.000124 | 0.032515 | 0 | 0 |
| gbc24 | 0 | 0 | 3.03E−05 | 0 | 0.000209 | 0 | 0.003624 | 0.001704 | 0 | 0 |
| gbc25 | 0 | 0 | 0 | 0 | 0.000484 | 0 | 0 | 0 | 0 | 0 |
| gbc26 | 0 | 0.00018 | 0.003059 | 0.000101 | 0 | 0.008272 | 0 | 0.000246 | 0 | 0 |
| gbc27 | 0 | 0 | 9.47E−05 | 0 | 0 | 0 | 0 | 0.000937 | 0 | 0 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | KIAA0586 | KIAA1324L | KIF3C | KLF10 | KLHDC1 | KLHDC8B | KLHL9 | KLRA1P | KRIT1 | LACTB2 |
|---|---|---|---|---|---|---|---|---|---|---|
| rf0 | 0.004271 | 0.006194 | 0.001836 | 0.000942 | 0 | 0.000377 | 0.003579 | 0 | 0 | 0.000917 |
| rf1 | 0 | 0.004422 | 0.000516 | 0 | 0.00094 | 0.010758 | 0.002581 | 0.000644 | 0 | 0.007893 |
| rf2 | 0.000398 | 0.003253 | 0.000623 | 1.15E−17 | 0.004028 | 7.32E−19 | 0.006318 | 0.003906 | 0.000784 | 0.001288 |
| rf3 | 0.003235 | 0.006553 | 0 | 0.002595 | 0.000101 | 0.002508 | 0.004152 | 0.002737 | 0.004068 | 0.001168 |
| rf4 | 0 | 0.003367 | 0 | 0.000768 | 4.28E−18 | 0.017486 | 0.002977 | 0.001385 | 0.00419 | 0.012506 |
| rf5 | 0 | 0.00572 | 0.005826 | 4.81E−18 | 0.001832 | 0.01454 | 0.006011 | 0 | 0.005974 | 0.009153 |
| rf6 | 0.003213 | 0.010009 | 0 | 0.000309 | 0.000387 | 0.001589 | 0.011408 | 0 | 0.0037 | 0.003989 |
| rf7 | 0.000371 | 0.01903 | 0.003128 | 0.000672 | 0.00105 | 0.00945 | 6.10E−18 | 0 | 0.003667 | 0.000958 |
| rf8 | 0.003634 | 0.006571 | 4.20E−19 | 0 | 0.001296 | 0.005623 | 0.001087 | 0.002601 | 0.003713 | 0.007444 |
| rf9 | 0.001025 | 0.004978 | 0 | 0 | 0 | 0.015742 | 0.007405 | 0.001036 | 0.000441 | 1.45E−18 |
| rf10 | 6.28E−19 | 0.00988 | 0 | 0.000661 | 0 | 0.004105 | 0.008005 | 0 | 0.001742 | 0.00094 |
| rf11 | 0.002524 | 0.003701 | 4.05E−05 | 0.000492 | 0 | 0.008014 | 0.002173 | 0.000403 | 0.001516 | 0.002333 |
| rf12 | 0.001174 | 0.00303 | 0.000319 | 0.004426 | 0.001868 | 0.001359 | 0.006215 | 0.000477 | 0 | 0.00284 |
| rf13 | 0.001265 | 0.007717 | 1.13E−17 | 0.001334 | 0.002262 | 0.021461 | 0.004404 | 0.000359 | 0.002744 | 0.000734 |
| rf14 | 0.004271 | 0.006194 | 0.001836 | 0.000942 | 0 | 0.000377 | 0.003579 | 0 | 0 | 0.000917 |
| rf15 | 0 | 0.004422 | 0.000516 | 0 | 0.00094 | 0.010758 | 0.002581 | 0.000644 | 0 | 0.007893 |
| rf16 | 0.000398 | 0.003253 | 0.000623 | 1.15E−17 | 0.004028 | 7.32E−19 | 0.006318 | 0.003906 | 0.000784 | 0.001288 |
| rf17 | 0.003235 | 0.006553 | 0 | 0.002595 | 0.000101 | 0.002508 | 0.004152 | 0.002737 | 0.004068 | 0.001168 |
| rf18 | 0 | 0.003367 | 0 | 0.000768 | 4.28E−18 | 0.017486 | 0.002977 | 0.001385 | 0.00419 | 0.012506 |
| rf19 | 0 | 0.00572 | 0.005826 | 4.81E−18 | 0.001832 | 0.01454 | 0.006011 | 0 | 0.005974 | 0.009153 |
| rf20 | 0.003213 | 0.010009 | 0 | 0.000309 | 0.000387 | 0.001589 | 0.011408 | 0 | 0.0037 | 0.003989 |
| rf21 | 0.000371 | 0.01903 | 0.003128 | 0.000672 | 0.00105 | 0.00945 | 6.10E−18 | 0 | 0.003667 | 0.000958 |
| rf22 | 0.003634 | 0.006571 | 4.20E−19 | 0 | 0.001296 | 0.005623 | 0.001087 | 0.002601 | 0.003713 | 0.007444 |
| rf23 | 0.001025 | 0.004978 | 0 | 0 | 0 | 0.015742 | 0.007405 | 0.001036 | 0.000441 | 1.45E−18 |
| rf24 | 6.28E−19 | 0.00988 | 0 | 0.000661 | 0 | 0.004105 | 0.008005 | 0 | 0.001742 | 0.00094 |
| rf25 | 0.002524 | 0.003701 | 4.05E−05 | 0.000492 | 0 | 0.008014 | 0.002173 | 0.000403 | 0.001516 | 0.002333 |
| rf26 | 0.001174 | 0.00303 | 0.000319 | 0.004426 | 0.001868 | 0.001359 | 0.006215 | 0.000477 | 0 | 0.00284 |
| rf27 | 0.001265 | 0.007717 | 1.13E−17 | 0.001334 | 0.002262 | 0.021461 | 0.004404 | 0.000359 | 0.002744 | 0.000734 |
| extraTrees0 | 0.000913 | 0.006305 | 0.001165 | 0.000644 | 0.001491 | 0.007273 | 0.000952 | 0.001935 | 0.00028 | 0.00197 |
| extraTrees1 | 0.001678 | 0.003648 | 0.002521 | 0.000434 | 0.00113 | 0.003837 | 0.007449 | 0.001125 | 0.001077 | 0.00314 |
| extraTrees2 | 0.002617 | 0.002923 | 0.003363 | 0.000484 | 0.001232 | 0.00665 | 0.004633 | 0.00067 | 0.001711 | 0.002767 |
| extraTrees3 | 2.53E−18 | 0.008273 | 0.000701 | 0.001192 | 0.002658 | 0.00553 | 0.005074 | 0.001266 | 0.003312 | 0.001695 |
| extraTrees4 | 0.002915 | 0.004662 | 0.003094 | 0.000356 | 0.000895 | 0.0065 | 0.008748 | 0.001722 | 0.003021 | 0.003451 |
| extraTrees5 | 0.0057 | 0.007175 | 0.003067 | 0.000837 | 0.000914 | 0.00479 | 0.006279 | 0.000863 | 0.000271 | 0.001846 |
| extraTrees6 | 0.000373 | 0.002799 | 2.77E−18 | 0 | 0.001161 | 0.010621 | 0.013257 | 0.00145 | 0.000371 | 0.004217 |
| extraTrees7 | 0.001461 | 0.004883 | 0.003549 | 3.28E−18 | 0.002496 | 0.004228 | 0.005201 | 0.000592 | 0.002491 | 0.003744 |
| extraTrees8 | 0.002927 | 0.006925 | 0.000515 | 1.06E−18 | 0.003953 | 0.005751 | 0.004957 | 0.000827 | 0.00207 | 0.005316 |
| extraTrees9 | 0.001869 | 0.003363 | 0.000291 | 0.000784 | 0.00205 | 0.008395 | 0.005853 | 0.001441 | 0.001243 | 0.004686 |
| extraTrees10 | 0.001132 | 0.007322 | 0.001343 | 6.83E−19 | 0.001293 | 0.002815 | 0.004304 | 0.000273 | 0.002068 | 0.006032 |
| extraTrees11 | 0.003376 | 0.006357 | 0.00318 | 5.26E−19 | 0.000291 | 0.004703 | 0.001825 | 5.53E−18 | 0.00256 | 0.00183 |
| extraTrees12 | 0.000238 | 0.005937 | 0.001871 | 0.000729 | 0 | 0.00904 | 0.00134 | 0.001312 | 0.000655 | 0.002625 |
| extraTrees13 | 0.000342 | 0.010283 | 0.000805 | 5.26E−19 | 0.000369 | 0.008218 | 0.004507 | 0.001741 | 0.000909 | 0.001627 |
| extraTrees14 | 0.000913 | 0.006305 | 0.001165 | 0.000644 | 0.001491 | 0.007273 | 0.000952 | 0.001935 | 0.00028 | 0.00197 |
| extraTrees15 | 0.001678 | 0.003648 | 0.002521 | 0.000434 | 0.00113 | 0.003837 | 0.007449 | 0.001125 | 0.001077 | 0.00314 |
| extraTrees16 | 0.002617 | 0.002923 | 0.003363 | 0.000484 | 0.001232 | 0.00665 | 0.004633 | 0.00067 | 0.001711 | 0.002767 |
| extraTrees17 | 2.53E−18 | 0.008273 | 0.000701 | 0.001192 | 0.002658 | 0.00553 | 0.005074 | 0.001266 | 0.003312 | 0.001695 |
| extraTrees18 | 0.002915 | 0.004662 | 0.003094 | 0.000356 | 0.000895 | 0.0065 | 0.008748 | 0.001722 | 0.003021 | 0.003451 |
| extraTrees19 | 0.0057 | 0.007175 | 0.003067 | 0.000837 | 0.000914 | 0.00479 | 0.006279 | 0.000863 | 0.000271 | 0.001846 |
| extraTrees20 | 0.000373 | 0.002799 | 2.77E−18 | 0 | 0.001161 | 0.010621 | 0.013257 | 0.00145 | 0.000371 | 0.004217 |
| extraTrees21 | 0.001461 | 0.004883 | 0.003549 | 3.28E−18 | 0.002496 | 0.004228 | 0.005201 | 0.000592 | 0.002491 | 0.003744 |
| extraTrees22 | 0.002927 | 0.006925 | 0.000515 | 1.06E−18 | 0.003953 | 0.005751 | 0.004957 | 0.000827 | 0.00207 | 0.005316 |
| extraTrees23 | 0.001869 | 0.003363 | 0.000291 | 0.000784 | 0.00205 | 0.008395 | 0.005853 | 0.001441 | 0.001243 | 0.004686 |
| extraTrees24 | 0.001132 | 0.007322 | 0.001343 | 6.83E−19 | 0.001293 | 0.002815 | 0.004304 | 0.000273 | 0.002068 | 0.006032 |
| extraTrees25 | 0.003376 | 0.006357 | 0.00318 | 5.26E−19 | 0.000291 | 0.004703 | 0.001825 | 5.53E−18 | 0.00256 | 0.00183 |
| extraTrees26 | 0.000238 | 0.005937 | 0.001871 | 0.000729 | 0 | 0.00904 | 0.00134 | 0.001312 | 0.000655 | 0.002625 |
| extraTrees27 | 0.000342 | 0.010283 | 0.000805 | 5.26E−19 | 0.000369 | 0.008218 | 0.004507 | 0.001741 | 0.000909 | 0.001627 |
| gbc0 | 0.002798 | 0.001943 | 0.035437 | 0 | 0 | 0 | 0.010394 | 0.025242 | 0.000277 | 0.000158 |
| gbc1 | 0.001159 | 0.004065 | 0.028167 | 0.000939 | 0.000638 | 0.014496 | 0.016908 | 0 | 5.81E−05 | 0.016285 |
| gbc2 | 0.011455 | 0.002638 | 0.000286 | 3.60E−05 | 0 | 0.006486 | 0 | 0 | 0.000962 | 0.015452 |
| gbc3 | 0.001043 | 0 | 6.74E−06 | 0 | 0.00198 | 0.000244 | 0.000218 | 0.001213 | 0.011515 | 0.028524 |
| gbc4 | 0.030487 | 0.000832 | 0 | 0 | 0.000101 | 0 | 0.013751 | 0 | 8.40E−05 | 0.000998 |
| gbc5 | 0.040264 | 0.005288 | 0.00915 | 0 | 0.000158 | 0.000136 | 0.00117 | 0 | 0 | 0.006952 |
| gbc6 | 0.02077 | 0.019487 | 3.01E−05 | 2.67E−05 | 0 | 0.011185 | 0.067763 | 0 | 0.001148 | 0.001661 |
| gbc7 | 0.001072 | 0.034148 | 0 | 5.40E−05 | 0 | 0.030404 | 2.95E−05 | 0 | 0.005848 | 0.001422 |
| gbc8 | 0.00065 | 6.22E−05 | 0.002233 | 0 | 0.003756 | 0.010759 | 0.028166 | 0 | 0 | 0 |
| gbc9 | 0.030801 | 0.000901 | 0.001588 | 0 | 0 | 0 | 0.004614 | 0.00585 | 4.20E−05 | 1.61E−05 |
| gbc10 | 0.009806 | 8.85E−05 | 0.003953 | 7.42E−05 | 0.003519 | 0.015906 | 0.00653 | 0.010331 | 9.34E−05 | 0.023454 |
| gbc11 | 0.023856 | 0 | 0.010388 | 6.27E−06 | 0.000822 | 0.000125 | 0.018009 | 0 | 0.012832 | 0.001745 |
| gbc12 | 0.00141 | 0.000242 | 0.016193 | 0 | 9.63E−05 | 0.003159 | 0.056457 | 0 | 0 | 0.008355 |
| gbc13 | 0.001168 | 0.021172 | 0.000141 | 0 | 0 | 0 | 0 | 2.16E−05 | 0.000591 | 0.003339 |
| gbc14 | 0.002798 | 0.001943 | 0.035437 | 0 | 0 | 0 | 0.010394 | 0.025242 | 0.000277 | 0.000158 |
| gbc15 | 0.001159 | 0.004065 | 0.028167 | 0.000939 | 0.000638 | 0.014496 | 0.016908 | 0 | 5.81E−05 | 0.016285 |
| gbc16 | 0.011455 | 0.002638 | 0.000286 | 3.60E−05 | 0 | 0.006486 | 0 | 0 | 0.000962 | 0.015452 |
| gbc17 | 0.001043 | 0 | 6.74E−06 | 0 | 0.00198 | 0.000244 | 0.000218 | 0.001213 | 0.011515 | 0.028524 |
| gbc18 | 0.030487 | 0.000832 | 0 | 0 | 0.000101 | 0 | 0.013751 | 0 | 8.40E−05 | 0.000998 |
| gbc19 | 0.040264 | 0.005288 | 0.00915 | 0 | 0.000158 | 0.000136 | 0.00117 | 0 | 0 | 0.006952 |
| gbc20 | 0.02077 | 0.019487 | 3.01E−05 | 2.67E−05 | 0 | 0.011185 | 0.067763 | 0 | 0.001148 | 0.001661 |
| gbc21 | 0.001072 | 0.034148 | 0 | 5.40E−05 | 0 | 0.030404 | 2.95E−05 | 0 | 0.005848 | 0.001422 |
| gbc22 | 0.00065 | 6.22E−05 | 0.002233 | 0 | 0.003756 | 0.010759 | 0.028166 | 0 | 0 | 0 |
| gbc23 | 0.030801 | 0.000901 | 0.001588 | 0 | 0 | 0 | 0.004614 | 0.00585 | 4.20E−05 | 1.61E−05 |
| gbc24 | 0.009806 | 8.85E−05 | 0.003953 | 7.42E−05 | 0.003519 | 0.015906 | 0.00653 | 0.010331 | 9.34E−05 | 0.023454 |
| gbc25 | 0.023856 | 0 | 0.010388 | 6.27E−06 | 0.000822 | 0.000125 | 0.018009 | 0 | 0.012832 | 0.001745 |
| gbc26 | 0.00141 | 0.000242 | 0.016193 | 0 | 9.63E−05 | 0.003159 | 0.056457 | 0 | 0 | 0.008355 |
| gbc27 | 0.001168 | 0.021172 | 0.000141 | 0 | 0 | 0 | 0 | 2.16E−05 | 0.000591 | 0.003339 |
| ada0 | 0 | 0.04 | 0 | 0 | 0 | 0 | 0.04 | 0 | 0 | 0.04 |
| ada1 | 0 | 0.04 | 0 | 0 | 0 | 0 | 0.04 | 0 | 0 | 0.04 |
| ada2 | 0 | 0.04 | 0 | 0 | 0 | 0 | 0.04 | 0 | 0 | 0.04 |
| ada3 | 0 | 0.04 | 0 | 0 | 0 | 0 | 0.04 | 0 | 0 | 0.04 |
| ada4 | 0 | 0.04 | 0 | 0 | 0 | 0 | 0.04 | 0 | 0 | 0.04 |
| ada5 | 0 | 0.04 | 0 | 0 | 0 | 0 | 0.04 | 0 | 0 | 0.04 |
| ada6 | 0 | 0.04 | 0 | 0 | 0 | 0 | 0.04 | 0 | 0 | 0.04 |
| ada7 | 0 | 0.04 | 0 | 0 | 0 | 0 | 0.04 | 0 | 0 | 0.04 |
| ada8 | 0 | 0.04 | 0 | 0 | 0 | 0 | 0.04 | 0 | 0 | 0.04 |
| ada9 | 0 | 0.04 | 0 | 0 | 0 | 0 | 0.04 | 0 | 0 | 0.04 |
| ada10 | 0 | 0.04 | 0 | 0 | 0 | 0 | 0.04 | 0 | 0 | 0.04 |
| ada11 | 0 | 0.04 | 0 | 0 | 0 | 0 | 0.04 | 0 | 0 | 0.04 |
| ada12 | 0 | 0.04 | 0 | 0 | 0 | 0 | 0.04 | 0 | 0 | 0.04 |
| ada13 | 0 | 0.04 | 0 | 0 | 0 | 0 | 0.04 | 0 | 0 | 0.04 |
| ada14 | 0 | 0.04 | 0 | 0 | 0 | 0 | 0.04 | 0 | 0 | 0.04 |
| ada15 | 0 | 0.04 | 0 | 0 | 0 | 0 | 0.04 | 0 | 0 | 0.04 |
| ada16 | 0 | 0.04 | 0 | 0 | 0 | 0 | 0.04 | 0 | 0 | 0.04 |
| ada17 | 0 | 0.04 | 0 | 0 | 0 | 0 | 0.04 | 0 | 0 | 0.04 |
| ada18 | 0 | 0.04 | 0 | 0 | 0 | 0 | 0.04 | 0 | 0 | 0.04 |
| ada19 | 0 | 0.04 | 0 | 0 | 0 | 0 | 0.04 | 0 | 0 | 0.04 |
| ada20 | 0 | 0.04 | 0 | 0 | 0 | 0 | 0.04 | 0 | 0 | 0.04 |
| ada21 | 0 | 0.04 | 0 | 0 | 0 | 0 | 0.04 | 0 | 0 | 0.04 |
| ada22 | 0 | 0.04 | 0 | 0 | 0 | 0 | 0.04 | 0 | 0 | 0.04 |
| ada23 | 0 | 0.04 | 0 | 0 | 0 | 0 | 0.04 | 0 | 0 | 0.04 |
| ada24 | 0 | 0.04 | 0 | 0 | 0 | 0 | 0.04 | 0 | 0 | 0.04 |
| ada25 | 0 | 0.04 | 0 | 0 | 0 | 0 | 0.04 | 0 | 0 | 0.04 |
| ada26 | 0 | 0.04 | 0 | 0 | 0 | 0 | 0.04 | 0 | 0 | 0.04 |
| ada27 | 0 | 0.04 | 0 | 0 | 0 | 0 | 0.04 | 0 | 0 | 0.04 |

| Gene | LCORL | LINC00867 | LINC00963 | LINC01218 | LINC02001 | LINC02284 | LMBRD2 | LSM14B | LYSMD3 | LYST |
|---|---|---|---|---|---|---|---|---|---|---|
| rf0 | 1.82E−17 | 0.000615 | 0.001939 | 0.008051 | 0.00583 | 0.001051 | 0.004606 | 0 | 0.000853 | 0 |
| rf1 | 0.001771 | 0.000833 | 0.001514 | 0.013253 | 0.000738 | 0 | 0.001388 | 0.003106 | 6.57E−19 | 0.001825 |
| rf2 | 0.000358 | 0.000643 | 0.006788 | 0.002363 | 0 | 0.000773 | 3.88E−18 | 0 | 0 | 0.001725 |
| rf3 | 0.00343 | 0.005986 | 0.004435 | 0.000362 | 1.08E−17 | 0 | 0.003054 | 0.001347 | 0.005976 | 0.006882 |
| rf4 | 0 | 0.004752 | 0.009739 | 0.0008 | 2.11E−18 | 0.002292 | 0.006935 | 0.002194 | 0.00034 | 0 |
| rf5 | 0 | 0.002907 | 0.008018 | 3.57E−19 | 0.000399 | 0.003473 | 0 | 0.00428 | 0.00035 | 0 |
| rf6 | 0.001469 | 0.009626 | 0.005635 | 0.002197 | 0.003089 | 0.001672 | 0.000932 | 0 | 0.001366 | 0.001024 |
| rf7 | 0.001906 | 0.006535 | 0.008558 | 0 | 0 | 0.000726 | 4.34E−19 | 7.98E−05 | 0.00037 | 0.001729 |
| rf8 | 0.001487 | 0.003092 | 0.011419 | 0.006879 | 0.002643 | 0.004373 | 0.000618 | 0 | 2.78E−05 | 0.001453 |
| rf9 | 0.000402 | 0.000403 | 0.006121 | 0.012336 | 0.000311 | 0 | 0.004884 | 0 | 0.004335 | 0.00528 |
| rf10 | 0 | 0 | 0.014702 | 0.004969 | 0.001521 | 0.000363 | 0.002768 | 2.40E−19 | 0 | 0 |
| rf11 | 0.006311 | 0.011632 | 0.004362 | 0.001075 | 0.000368 | 2.36E−18 | 0.004396 | 0.002467 | 0.002738 | 0 |
| rf12 | 0 | 0.005514 | 0.004177 | 0.002752 | 0.004254 | 0.002895 | 0.004958 | 1.66E−18 | 0.000359 | 0 |
| rf13 | 0 | 0.005738 | 0.002988 | 0 | 0.000935 | 0.002362 | 0.004505 | 5.66E−19 | 0.00069 | 0.006753 |
| rf14 | 1.82E−17 | 0.000615 | 0.001939 | 0.008051 | 0.00583 | 0.001051 | 0.004606 | 0 | 0.000853 | 0 |
| rf15 | 0.001771 | 0.000833 | 0.001514 | 0.013253 | 0.000738 | 0 | 0.001388 | 0.003106 | 6.57E−19 | 0.001825 |
| rf16 | 0.000358 | 0.000643 | 0.006788 | 0.002363 | 0 | 0.000773 | 3.88E−18 | 0 | 0 | 0.001725 |
| rf17 | 0.00343 | 0.005986 | 0.004435 | 0.000362 | 1.08E−17 | 0 | 0.003054 | 0.001347 | 0.005976 | 0.006882 |
| rf18 | 0 | 0.004752 | 0.009739 | 0.0008 | 2.11E−18 | 0.002292 | 0.006935 | 0.002194 | 0.00034 | 0 |
| rf19 | 0 | 0.002907 | 0.008018 | 3.57E−19 | 0.000399 | 0.003473 | 0 | 0.00428 | 0.00035 | 0 |
| rf20 | 0.001469 | 0.009626 | 0.005635 | 0.002197 | 0.003089 | 0.001672 | 0.000932 | 0 | 0.001366 | 0.001024 |
| rf21 | 0.001906 | 0.006535 | 0.008558 | 0 | 0 | 0.000726 | 4.34E−19 | 7.98E−05 | 0.00037 | 0.001729 |
| rf22 | 0.001487 | 0.003092 | 0.011419 | 0.006879 | 0.002643 | 0.004373 | 0.000618 | 0 | 2.78E−05 | 0.001453 |
| rf23 | 0.000402 | 0.000403 | 0.006121 | 0.012336 | 0.000311 | 0 | 0.004884 | 0 | 0.004335 | 0.00528 |
| rf24 | 0 | 0 | 0.014702 | 0.004969 | 0.001521 | 0.000363 | 0.002768 | 2.40E−19 | 0 | 0 |
| rf25 | 0.006311 | 0.011632 | 0.004362 | 0.001075 | 0.000368 | 2.36E−18 | 0.004396 | 0.002467 | 0.002738 | 0 |
| rf26 | 0 | 0.005514 | 0.004177 | 0.002752 | 0.004254 | 0.002895 | 0.004958 | 1.66E−18 | 0.000359 | 0 |
| rf27 | 0 | 0.005738 | 0.002988 | 0 | 0.000935 | 0.002362 | 0.004505 | 5.66E−19 | 0.00069 | 0.006753 |
| extraTrees0 | 4.25E−18 | 0.006536 | 0.005129 | 0.004021 | 0.000238 | 0.002289 | 0.003253 | 0.000642 | 0.002131 | 0.002409 |
| extraTrees1 | 0.002392 | 0.002638 | 0.003209 | 0.00499 | 0.001115 | 0.003295 | 0.001794 | 0.003071 | 0.000807 | 0.000669 |
| extraTrees2 | 0.003037 | 0.002823 | 0.010557 | 0.002732 | 0.003044 | 0.000736 | 0.000656 | 0.001613 | 0.001628 | 0.000446 |
| extraTrees3 | 0.001651 | 0.003132 | 0.00746 | 0.002895 | 0.003253 | 0.00104 | 0.0014 | 0.000771 | 0.000621 | 0.001217 |
| extraTrees4 | 0.000708 | 0.008201 | 0.007888 | 0.004644 | 0.001129 | 0.000999 | 0.00073 | 0.001626 | 0.000872 | 0.000757 |
| extraTrees5 | 0.00144 | 0.007606 | 0.009444 | 0.00071 | 0.002921 | 0.001947 | 0.001221 | 0.003431 | 0.000679 | 0.001033 |
| extraTrees6 | 0.000636 | 0.005942 | 0.009675 | 0.00396 | 0.003328 | 0.00489 | 0.00181 | 0.000664 | 0.001974 | 0.00062 |
| extraTrees7 | 0.002629 | 0.001389 | 0.005668 | 0.002121 | 0.004223 | 0.002989 | 0.000388 | 0.004133 | 0.001359 | 0.002869 |
| extraTrees8 | 0.000293 | 0.002088 | 0.005757 | 0.001809 | 0.000847 | 0.002568 | 0.001865 | 0.002168 | 0.001701 | 0.001786 |
| extraTrees9 | 0.001807 | 0.004636 | 0.008156 | 0.004059 | 0.004505 | 0.002609 | 0.001768 | 0.001243 | 0.001883 | 0.001263 |
| extraTrees10 | 0.001267 | 0.004576 | 0.005661 | 0.003394 | 0.002778 | 0.001358 | 0.003418 | 0.001705 | 0.001078 | 0.000875 |
| extraTrees11 | 0.001413 | 0.009752 | 0.008275 | 0.001159 | 0.002393 | 0.003228 | 0.005219 | 0.000821 | 0.000653 | 0.000964 |
| extraTrees12 | 8.20E−19 | 0.002085 | 0.006626 | 0.002724 | 0.003037 | 0.001732 | 0.003095 | 0.001781 | 0.002131 | 0.002392 |
| extraTrees13 | 0.001664 | 0.003247 | 0.004081 | 0.003216 | 0.003276 | 0.004605 | 0.003718 | 0.000478 | 0.001145 | 0.003142 |
| extraTrees14 | 4.25E−18 | 0.006536 | 0.005129 | 0.004021 | 0.000238 | 0.002289 | 0.003253 | 0.000642 | 0.002131 | 0.002409 |
| extraTrees15 | 0.002392 | 0.002638 | 0.003209 | 0.00499 | 0.001115 | 0.003295 | 0.001794 | 0.003071 | 0.000807 | 0.000669 |
| extraTrees16 | 0.003037 | 0.002823 | 0.010557 | 0.002732 | 0.003044 | 0.000736 | 0.000656 | 0.001613 | 0.001628 | 0.000446 |
| extraTrees17 | 0.001651 | 0.003132 | 0.00746 | 0.002895 | 0.003253 | 0.00104 | 0.0014 | 0.000771 | 0.000621 | 0.001217 |
| extraTrees18 | 0.000708 | 0.008201 | 0.007888 | 0.004644 | 0.001129 | 0.000999 | 0.00073 | 0.001626 | 0.000872 | 0.000757 |
| extraTrees19 | 0.00144 | 0.007606 | 0.009444 | 0.00071 | 0.002921 | 0.001947 | 0.001221 | 0.003431 | 0.000679 | 0.001033 |
| extraTrees20 | 0.000636 | 0.005942 | 0.009675 | 0.00396 | 0.003328 | 0.00489 | 0.00181 | 0.000664 | 0.001974 | 0.00062 |
| extraTrees21 | 0.002629 | 0.001389 | 0.005668 | 0.002121 | 0.004223 | 0.002989 | 0.000388 | 0.004133 | 0.001359 | 0.002869 |
| extraTrees22 | 0.000293 | 0.002088 | 0.005757 | 0.001809 | 0.000847 | 0.002568 | 0.001865 | 0.002168 | 0.001701 | 0.001786 |
| extraTrees23 | 0.001807 | 0.004636 | 0.008156 | 0.004059 | 0.004505 | 0.002609 | 0.001768 | 0.001243 | 0.001883 | 0.001263 |
| extraTrees24 | 0.001267 | 0.004576 | 0.005661 | 0.003394 | 0.002778 | 0.001358 | 0.003418 | 0.001705 | 0.001078 | 0.000875 |
| extraTrees25 | 0.001413 | 0.009752 | 0.008275 | 0.001159 | 0.002393 | 0.003228 | 0.005219 | 0.000821 | 0.000653 | 0.000964 |
| extraTrees26 | 8.20E−19 | 0.002085 | 0.006626 | 0.002724 | 0.003037 | 0.001732 | 0.003095 | 0.001781 | 0.002131 | 0.002392 |
| extraTrees27 | 0.001664 | 0.003247 | 0.004081 | 0.003216 | 0.003276 | 0.004605 | 0.003718 | 0.000478 | 0.001145 | 0.003142 |
| gbc0 | 0 | 0.001001 | 0.011622 | 0 | 0.00176 | 0.000491 | 0 | 0.005688 | 0 | 3.69E−06 |
| gbc1 | 0 | 1.98E−06 | 0.005398 | 0.019883 | 0.007268 | 0 | 0 | 0 | 0 | 0 |
| gbc2 | 0 | 0 | 5.52E−07 | 0 | 0 | 0.000332 | 1.51E−05 | 0 | 0 | 0.001354 |
| gbc3 | 0 | 0.000179 | 0 | 0 | 0 | 5.04E−05 | 0 | 0.020258 | 3.59E−05 | 0 |
| gbc4 | 6.58E−06 | 0 | 0.02032 | 1.16E−05 | 9.38E−06 | 0.000905 | 0 | 1.09E−07 | 0 | 0 |
| gbc5 | 0.002911 | 3.11E−05 | 0.000177 | 0 | 0.016375 | 9.68E−05 | 0 | 0.000104 | 0 | 0.001861 |
| gbc6 | 0.001402 | 0.008815 | 0.018654 | 0.000657 | 0.000243 | 0 | 8.32E−06 | 4.76E−07 | 0 | 0.000769 |
| gbc7 | 0 | 0.013465 | 0.012443 | 0 | 1.92E−05 | 0.000102 | 0.002603 | 4.86E−06 | 1.82E−06 | 0.020479 |
| gbc8 | 0 | 0 | 0.000132 | 0 | 0.000192 | 0 | 0.007664 | 0.011827 | 0 | 0 |
| gbc9 | 0 | 0.014952 | 0 | 0 | 0 | 0.005989 | 0 | 0.000342 | 7.16E−06 | 8.86E−05 |
| gbc10 | 0 | 0.012948 | 0.001411 | 0 | 0 | 3.96E−05 | 0.001432 | 0 | 0.004302 | 0 |
| gbc11 | 0 | 0.000684 | 0 | 0.000139 | 0 | 0.00444 | 0 | 2.53E−05 | 0 | 0 |
| gbc12 | 0.000462 | 0 | 0 | 0 | 9.04E−05 | 0 | 0 | 0.00023 | 0 | 0.003556 |
| gbc13 | 0 | 0.00748 | 8.31E−05 | 0 | 0.001761 | 0 | 0.003041 | 9.07E−06 | 0 | 7.20E−05 |
| gbc14 | 0 | 0.001001 | 0.011622 | 0 | 0.00176 | 0.000491 | 0 | 0.005688 | 0 | 3.69E−06 |
| gbc15 | 0 | 1.98E−06 | 0.005398 | 0.019883 | 0.007268 | 0 | 0 | 0 | 0 | 0 |
| gbc16 | 0 | 0 | 5.52E−07 | 0 | 0 | 0.000332 | 1.51E−05 | 0 | 0 | 0.001354 |
| gbc17 | 0 | 0.000179 | 0 | 0 | 0 | 5.04E−05 | 0 | 0.020258 | 3.59E−05 | 0 |
| gbc18 | 6.58E−06 | 0 | 0.02032 | 1.16E−05 | 9.38E−06 | 0.000905 | 0 | 1.09E−07 | 0 | 0 |
| gbc19 | 0.002911 | 3.11E−05 | 0.000177 | 0 | 0.016375 | 9.68E−05 | 0 | 0.000104 | 0 | 0.001861 |
| gbc20 | 0.001402 | 0.008815 | 0.018654 | 0.000657 | 0.000243 | 0 | 8.32E−06 | 4.76E−07 | 0 | 0.000769 |
| gbc21 | 0 | 0.013465 | 0.012443 | 0 | 1.92E−05 | 0.000102 | 0.002603 | 4.86E−06 | 1.82E−06 | 0.020479 |
| gbc22 | 0 | 0 | 0.000132 | 0 | 0.000192 | 0 | 0.007664 | 0.011827 | 0 | 0 |
| gbc23 | 0 | 0.014952 | 0 | 0 | 0 | 0.005989 | 0 | 0.000342 | 7.16E−06 | 8.86E−05 |
| gbc24 | 0 | 0.012948 | 0.001411 | 0 | 0 | 3.96E−05 | 0.001432 | 0 | 0.004302 | 0 |
| gbc25 | 0 | 0.000684 | 0 | 0.000139 | 0 | 0.00444 | 0 | 2.53E−05 | 0 | 0 |
| gbc26 | 0.000462 | 0 | 0 | 0 | 9.04E−05 | 0 | 0 | 0.00023 | 0 | 0.003556 |
| gbc27 | 0 | 0.00748 | 8.31E−05 | 0 | 0.001761 | 0 | 0.003041 | 9.07E−06 | 0 | 7.20E−05 |
| ada0 | 0 | 0 | 0 | 0 | 0 | 0 | 0 | 0.02 | 0 | 0 |
| ada1 | 0 | 0 | 0 | 0 | 0 | 0 | 0 | 0.02 | 0 | 0 |
| ada2 | 0 | 0 | 0 | 0 | 0 | 0 | 0 | 0.02 | 0 | 0 |
| ada3 | 0 | 0 | 0 | 0 | 0 | 0 | 0 | 0.02 | 0 | 0 |
| ada4 | 0 | 0 | 0 | 0 | 0 | 0 | 0 | 0.02 | 0 | 0 |
| ada5 | 0 | 0 | 0 | 0 | 0 | 0 | 0 | 0.02 | 0 | 0 |
| ada6 | 0 | 0 | 0 | 0 | 0 | 0 | 0 | 0.02 | 0 | 0 |
| ada7 | 0 | 0 | 0 | 0 | 0 | 0 | 0 | 0.02 | 0 | 0 |
| ada8 | 0 | 0 | 0 | 0 | 0 | 0 | 0 | 0.02 | 0 | 0 |
| ada9 | 0 | 0 | 0 | 0 | 0 | 0 | 0 | 0.02 | 0 | 0 |
| ada10 | 0 | 0 | 0 | 0 | 0 | 0 | 0 | 0.02 | 0 | 0 |
| ada11 | 0 | 0 | 0 | 0 | 0 | 0 | 0 | 0.02 | 0 | 0 |
| ada12 | 0 | 0 | 0 | 0 | 0 | 0 | 0 | 0.02 | 0 | 0 |
| ada13 | 0 | 0 | 0 | 0 | 0 | 0 | 0 | 0.02 | 0 | 0 |
| ada14 | 0 | 0 | 0 | 0 | 0 | 0 | 0 | 0.02 | 0 | 0 |
| ada15 | 0 | 0 | 0 | 0 | 0 | 0 | 0 | 0.02 | 0 | 0 |
| ada16 | 0 | 0 | 0 | 0 | 0 | 0 | 0 | 0.02 | 0 | 0 |
| ada17 | 0 | 0 | 0 | 0 | 0 | 0 | 0 | 0.02 | 0 | 0 |
| ada18 | 0 | 0 | 0 | 0 | 0 | 0 | 0 | 0.02 | 0 | 0 |
| ada19 | 0 | 0 | 0 | 0 | 0 | 0 | 0 | 0.02 | 0 | 0 |
| ada20 | 0 | 0 | 0 | 0 | 0 | 0 | 0 | 0.02 | 0 | 0 |
| ada21 | 0 | 0 | 0 | 0 | 0 | 0 | 0 | 0.02 | 0 | 0 |
| ada22 | 0 | 0 | 0 | 0 | 0 | 0 | 0 | 0.02 | 0 | 0 |
| ada23 | 0 | 0 | 0 | 0 | 0 | 0 | 0 | 0.02 | 0 | 0 |
| ada24 | 0 | 0 | 0 | 0 | 0 | 0 | 0 | 0.02 | 0 | 0 |
| ada25 | 0 | 0 | 0 | 0 | 0 | 0 | 0 | 0.02 | 0 | 0 |
| ada26 | 0 | 0 | 0 | 0 | 0 | 0 | 0 | 0.02 | 0 | 0 |
| ada27 | 0 | 0 | 0 | 0 | 0 | 0 | 0 | 0.02 | 0 | 0 |

| Gene | MAP1A | MAPKAPK2 | MBNL2 | MECP2 | MED16 | MEMO1P1 | METTL7A | MFAP3 | MINDY1 | MLLT11 |
|---|---|---|---|---|---|---|---|---|---|---|
| rf0 | 0 | 0.000755 | 0.003819 | 0.008471 | 0.002772 | 0.001409 | 0.002276 | 0 | 0.002319 | 0.003631 |
| rf1 | 0 | 0.004213 | 0.001764 | 0.007433 | 0.000987 | 0 | 0.009764 | 0.0013 | 0 | 0.000475 |
| rf2 | 0.003089 | 0 | 9.45E−18 | 0 | 0 | 0.003453 | 0.003257 | 0.004012 | 0 | 0 |
| rf3 | 0.00033 | 0.000353 | 0.001574 | 0.005843 | 0.00179 | 0.000659 | 0 | 0.00035 | 0.002428 | 0.000372 |
| rf4 | 5.82E−18 | 0.008404 | 0 | 0.000284 | 0.004 | 0.001096 | 0.001133 | 0 | 4.47E−18 | 0.001225 |
| rf5 | 0.00156 | 0.000902 | 0.002527 | 0.004056 | 0 | 0.000361 | 0.003737 | 0.006643 | 0 | 0 |
| rf6 | 5.84E−18 | 0.00491 | 0 | 0.001397 | 0 | 0 | 0.007359 | 0 | 0.000393 | 0.003286 |
| rf7 | 0.000415 | 0.00246 | 0.001556 | 0.004054 | 0 | 0.00405 | 0.006027 | 0.00134 | 0.000759 | 0.003497 |
| rf8 | 0.003508 | 0.008467 | 0.003233 | 0 | 0.003585 | 0.000684 | 0 | 0.001848 | 0.002922 | 0.001803 |
| rf9 | 0.005302 | 0.004904 | 0.00033 | 0.004441 | 0 | 0.002785 | 6.97E−19 | 0.001038 | 0 | 0.001426 |
| rf10 | 0.001248 | 0.007415 | 0.000489 | 0 | 4.36E−17 | 0.002254 | 0.000801 | 0.001606 | 0 | 0.001861 |
| rf11 | 3.87E−18 | 0.001875 | 0 | 0.000391 | 0 | 0.002449 | 0.002181 | 0 | 0.003332 | 0.001244 |
| rf12 | 0 | 0.006009 | 0.001518 | 0.000985 | 0.000665 | 0.001932 | 0.00265 | 0 | 0 | 0 |
| rf13 | 0.004312 | 0.005853 | 0.003113 | 0.002549 | 0.005686 | 0.002051 | 0.000845 | 0.001241 | 0.008268 | 0 |
| rf14 | 0 | 0.000755 | 0.003819 | 0.008471 | 0.002772 | 0.001409 | 0.002276 | 0 | 0.002319 | 0.003631 |
| rf15 | 0 | 0.004213 | 0.001764 | 0.007433 | 0.000987 | 0 | 0.009764 | 0.0013 | 0 | 0.000475 |
| rf16 | 0.003089 | 0 | 9.45E−18 | 0 | 0 | 0.003453 | 0.003257 | 0.004012 | 0 | 0 |
| rf17 | 0.00033 | 0.000353 | 0.001574 | 0.005843 | 0.00179 | 0.000659 | 0 | 0.00035 | 0.002428 | 0.000372 |
| rf18 | 5.82E−18 | 0.008404 | 0 | 0.000284 | 0.004 | 0.001096 | 0.001133 | 0 | 4.47E−18 | 0.001225 |
| rf19 | 0.00156 | 0.000902 | 0.002527 | 0.004056 | 0 | 0.000361 | 0.003737 | 0.006643 | 0 | 0 |
| rf20 | 5.84E−18 | 0.00491 | 0 | 0.001397 | 0 | 0 | 0.007359 | 0 | 0.000393 | 0.003286 |
| rf21 | 0.000415 | 0.00246 | 0.001556 | 0.004054 | 0 | 0.00405 | 0.006027 | 0.00134 | 0.000759 | 0.003497 |
| rf22 | 0.003508 | 0.008467 | 0.003233 | 0 | 0.003585 | 0.000684 | 0 | 0.001848 | 0.002922 | 0.001803 |
| rf23 | 0.005302 | 0.004904 | 0.00033 | 0.004441 | 0 | 0.002785 | 6.97E−19 | 0.001038 | 0 | 0.001426 |
| rf24 | 0.001248 | 0.007415 | 0.000489 | 0 | 4.36E−17 | 0.002254 | 0.000801 | 0.001606 | 0 | 0.001861 |
| rf25 | 3.87E−18 | 0.001875 | 0 | 0.000391 | 0 | 0.002449 | 0.002181 | 0 | 0.003332 | 0.001244 |
| rf26 | 0 | 0.006009 | 0.001518 | 0.000985 | 0.000665 | 0.001932 | 0.00265 | 0 | 0 | 0 |
| rf27 | 0.004312 | 0.005853 | 0.003113 | 0.002549 | 0.005686 | 0.002051 | 0.000845 | 0.001241 | 0.008268 | 0 |
| extraTrees0 | 0.000267 | 0 | 0.000948 | 0.001917 | 0.002395 | 0.000718 | 0.002498 | 0.001636 | 0.002804 | 0.000238 |
| extraTrees1 | 0.001668 | 0.001431 | 0.001766 | 0.000991 | 0.003505 | 0.001099 | 0.002248 | 0.000492 | 0.002776 | 0.004107 |
| extraTrees2 | 0.001851 | 0.005929 | 0.00112 | 0.000907 | 0.001945 | 0.000507 | 0.001453 | 0.003651 | 3.17E−18 | 0.000892 |
| extraTrees3 | 0.001248 | 0.000268 | 0.00188 | 0.001793 | 0.004036 | 0.002203 | 0.001045 | 0.002274 | 0.000718 | 0.001766 |
| extraTrees4 | 0.001236 | 0.002338 | 0.003614 | 0.000736 | 0.000415 | 0.001596 | 0.000526 | 0.002457 | 0.0017 | 0.001569 |
| extraTrees5 | 0.000805 | 2.12E−18 | 0.002561 | 0.001405 | 0.00214 | 0.00202 | 0.001598 | 0.001429 | 0.003336 | 0.001146 |
| extraTrees6 | 0.001725 | 6.15E−19 | 0.000936 | 0.000717 | 0.003317 | 0.001755 | 0.001019 | 0.000757 | 0.004135 | 0.000573 |
| extraTrees7 | 0.002337 | 0.004886 | 0.000868 | 0.002237 | 0.001165 | 0 | 0.002641 | 0.002027 | 0.004502 | 0.000629 |
| extraTrees8 | 0.002799 | 0.002191 | 0.001946 | 0 | 0.00239 | 0.001829 | 0.002776 | 0.001794 | 0.002131 | 0.004019 |
| extraTrees9 | 0.003292 | 0.002144 | 0.000786 | 0.002604 | 0.001652 | 0.000757 | 0.001639 | 0.006013 | 0.000334 | 0.000858 |
| extraTrees10 | 0.001901 | 0.004842 | 0.000469 | 0.002081 | 0.003075 | 0 | 0.004595 | 0.003249 | 0.001718 | 1.54E−18 |
| extraTrees11 | 0.00135 | 0.004719 | 0.001304 | 0.002211 | 0.00178 | 0.001297 | 0.003256 | 0.001841 | 0.000395 | 0.001155 |
| extraTrees12 | 0.000268 | 0.002495 | 0.000789 | 0.001308 | 0.000291 | 0.000238 | 0.001793 | 0.002283 | 0.001666 | 0.001688 |
| extraTrees13 | 0.003153 | 0.000274 | 0.001376 | 2.73E−19 | 0.001292 | 0.000923 | 0.000839 | 0.001274 | 0.001503 | 0.001748 |
| extraTrees14 | 0.000267 | 0 | 0.000948 | 0.001917 | 0.002395 | 0.000718 | 0.002498 | 0.001636 | 0.002804 | 0.000238 |
| extraTrees15 | 0.001668 | 0.001431 | 0.001766 | 0.000991 | 0.003505 | 0.001099 | 0.002248 | 0.000492 | 0.002776 | 0.004107 |
| extraTrees16 | 0.001851 | 0.005929 | 0.00112 | 0.000907 | 0.001945 | 0.000507 | 0.001453 | 0.003651 | 3.17E−18 | 0.000892 |
| extraTrees17 | 0.001248 | 0.000268 | 0.00188 | 0.001793 | 0.004036 | 0.002203 | 0.001045 | 0.002274 | 0.000718 | 0.001766 |
| extraTrees18 | 0.001236 | 0.002338 | 0.003614 | 0.000736 | 0.000415 | 0.001596 | 0.000526 | 0.002457 | 0.0017 | 0.001569 |
| extraTrees19 | 0.000805 | 2.12E−18 | 0.002561 | 0.001405 | 0.00214 | 0.00202 | 0.001598 | 0.001429 | 0.003336 | 0.001146 |
| extraTrees20 | 0.001725 | 6.15E−19 | 0.000936 | 0.000717 | 0.003317 | 0.001755 | 0.001019 | 0.000757 | 0.004135 | 0.000573 |
| extraTrees21 | 0.002337 | 0.004886 | 0.000868 | 0.002237 | 0.001165 | 0 | 0.002641 | 0.002027 | 0.004502 | 0.000629 |
| extraTrees22 | 0.002799 | 0.002191 | 0.001946 | 0 | 0.00239 | 0.001829 | 0.002776 | 0.001794 | 0.002131 | 0.004019 |
| extraTrees23 | 0.003292 | 0.002144 | 0.000786 | 0.002604 | 0.001652 | 0.000757 | 0.001639 | 0.006013 | 0.000334 | 0.000858 |
| extraTrees24 | 0.001901 | 0.004842 | 0.000469 | 0.002081 | 0.003075 | 0 | 0.004595 | 0.003249 | 0.001718 | 1.54E−18 |
| extraTrees25 | 0.00135 | 0.004719 | 0.001304 | 0.002211 | 0.00178 | 0.001297 | 0.003256 | 0.001841 | 0.000395 | 0.001155 |
| extraTrees26 | 0.000268 | 0.002495 | 0.000789 | 0.001308 | 0.000291 | 0.000238 | 0.001793 | 0.002283 | 0.001666 | 0.001688 |
| extraTrees27 | 0.003153 | 0.000274 | 0.001376 | 2.73E−19 | 0.001292 | 0.000923 | 0.000839 | 0.001274 | 0.001503 | 0.001748 |
| gbc0 | 0 | 0 | 0 | 0 | 0.001021 | 0 | 0 | 0 | 0 | 0.000552 |
| gbc1 | 0.000619 | 1.75E−05 | 0 | 0 | 0 | 0 | 3.41E−05 | 0 | 0 | 0 |
| gbc2 | 0 | 0 | 0 | 0 | 0 | 0.000634 | 0.01558 | 0 | 0 | 0.000477 |
| gbc3 | 0 | 0.01221 | 0 | 0.011127 | 0 | 0.002289 | 0 | 0 | 0 | 0.000297 |
| gbc4 | 0 | 0 | 2.69E−05 | 0.003178 | 9.12E−05 | 0.030493 | 0 | 0 | 0 | 0 |
| gbc5 | 2.04E−05 | 0 | 0.000804 | 0.003485 | 0.0002 | 0.001612 | 0 | 0 | 0 | 0.000673 |
| gbc6 | 0 | 0 | 0 | 0.007325 | 0 | 2.56E−05 | 5.31E−05 | 0 | 0 | 0.002107 |
| gbc7 | 0.002927 | 0 | 0.00031 | 0 | 0 | 0 | 0.002492 | 0 | 1.38E−05 | 0.000103 |
| gbc8 | 0.015559 | 0.007607 | 0 | 0.000526 | 0 | 0 | 0 | 0 | 0 | 0 |
| gbc9 | 0 | 0 | 0 | 0 | 0 | 0 | 0 | 0 | 0 | 0.000349 |
| gbc10 | 0 | 0.007031 | 0 | 0.001795 | 0.002748 | 0 | 0 | 1.66E−05 | 0.009024 | 0 |
| gbc11 | 0 | 0.001226 | 0 | 7.78E−05 | 0 | 6.28E−05 | 0.011477 | 0 | 0 | 1.86E−05 |
| gbc12 | 0 | 0.000416 | 0 | 1.30E−07 | 0.010201 | 0 | 5.83E−05 | 0 | 0.000113 | 6.34E−07 |
| gbc13 | 0.010496 | 0 | 0 | 1.33E−05 | 0 | 0 | 5.90E−05 | 0 | 0 | 2.08E−05 |
| gbc14 | 0 | 0 | 0 | 0 | 0.001021 | 0 | 0 | 0 | 0 | 0.000552 |
| gbc15 | 0.000619 | 1.75E−05 | 0 | 0 | 0 | 0 | 3.41E−05 | 0 | 0 | 0 |
| gbc16 | 0 | 0 | 0 | 0 | 0 | 0.000634 | 0.01558 | 0 | 0 | 0.000477 |
| gbc17 | 0 | 0.01221 | 0 | 0.011127 | 0 | 0.002289 | 0 | 0 | 0 | 0.000297 |
| gbc18 | 0 | 0 | 2.69E−05 | 0.003178 | 9.12E−05 | 0.030493 | 0 | 0 | 0 | 0 |
| gbc19 | 2.04E−05 | 0 | 0.000804 | 0.003485 | 0.0002 | 0.001612 | 0 | 0 | 0 | 0.000673 |
| gbc20 | 0 | 0 | 0 | 0.007325 | 0 | 2.56E−05 | 5.31E−05 | 0 | 0 | 0.002107 |
| gbc21 | 0.002927 | 0 | 0.00031 | 0 | 0 | 0 | 0.002492 | 0 | 1.38E−05 | 0.000103 |
| gbc22 | 0.015559 | 0.007607 | 0 | 0.000526 | 0 | 0 | 0 | 0 | 0 | 0 |
| gbc23 | 0 | 0 | 0 | 0 | 0 | 0 | 0 | 0 | 0 | 0.000349 |
| gbc24 | 0 | 0.007031 | 0 | 0.001795 | 0.002748 | 0 | 0 | 1.66E−05 | 0.009024 | 0 |
| gbc25 | 0 | 0.001226 | 0 | 7.78E−05 | 0 | 6.28E−05 | 0.011477 | 0 | 0 | 1.86E−05 |
| gbc26 | 0 | 0.000416 | 0 | 1.30E−07 | 0.010201 | 0 | 5.83E−05 | 0 | 0.000113 | 6.34E−07 |
| gbc27 | 0.010496 | 0 | 0 | 1.33E−05 | 0 | 0 | 5.90E−05 | 0 | 0 | 2.08E−05 |
| ada0 | 0 | 0.02 | 0 | 0 | 0 | 0 | 0 | 0 | 0 | 0 |
| ada1 | 0 | 0.02 | 0 | 0 | 0 | 0 | 0 | 0 | 0 | 0 |
| ada2 | 0 | 0.02 | 0 | 0 | 0 | 0 | 0 | 0 | 0 | 0 |
| ada3 | 0 | 0.02 | 0 | 0 | 0 | 0 | 0 | 0 | 0 | 0 |
| ada4 | 0 | 0.02 | 0 | 0 | 0 | 0 | 0 | 0 | 0 | 0 |
| ada5 | 0 | 0.02 | 0 | 0 | 0 | 0 | 0 | 0 | 0 | 0 |
| ada6 | 0 | 0.02 | 0 | 0 | 0 | 0 | 0 | 0 | 0 | 0 |
| ada7 | 0 | 0.02 | 0 | 0 | 0 | 0 | 0 | 0 | 0 | 0 |
| ada8 | 0 | 0.02 | 0 | 0 | 0 | 0 | 0 | 0 | 0 | 0 |
| ada9 | 0 | 0.02 | 0 | 0 | 0 | 0 | 0 | 0 | 0 | 0 |
| ada10 | 0 | 0.02 | 0 | 0 | 0 | 0 | 0 | 0 | 0 | 0 |
| ada11 | 0 | 0.02 | 0 | 0 | 0 | 0 | 0 | 0 | 0 | 0 |
| ada12 | 0 | 0.02 | 0 | 0 | 0 | 0 | 0 | 0 | 0 | 0 |
| ada13 | 0 | 0.02 | 0 | 0 | 0 | 0 | 0 | 0 | 0 | 0 |
| ada14 | 0 | 0.02 | 0 | 0 | 0 | 0 | 0 | 0 | 0 | 0 |
| ada15 | 0 | 0.02 | 0 | 0 | 0 | 0 | 0 | 0 | 0 | 0 |
| ada16 | 0 | 0.02 | 0 | 0 | 0 | 0 | 0 | 0 | 0 | 0 |
| ada17 | 0 | 0.02 | 0 | 0 | 0 | 0 | 0 | 0 | 0 | 0 |
| ada18 | 0 | 0.02 | 0 | 0 | 0 | 0 | 0 | 0 | 0 | 0 |
| ada19 | 0 | 0.02 | 0 | 0 | 0 | 0 | 0 | 0 | 0 | 0 |
| ada20 | 0 | 0.02 | 0 | 0 | 0 | 0 | 0 | 0 | 0 | 0 |
| ada21 | 0 | 0.02 | 0 | 0 | 0 | 0 | 0 | 0 | 0 | 0 |
| ada22 | 0 | 0.02 | 0 | 0 | 0 | 0 | 0 | 0 | 0 | 0 |
| ada23 | 0 | 0.02 | 0 | 0 | 0 | 0 | 0 | 0 | 0 | 0 |
| ada24 | 0 | 0.02 | 0 | 0 | 0 | 0 | 0 | 0 | 0 | 0 |
| ada25 | 0 | 0.02 | 0 | 0 | 0 | 0 | 0 | 0 | 0 | 0 |
| ada26 | 0 | 0.02 | 0 | 0 | 0 | 0 | 0 | 0 | 0 | 0 |
| ada27 | 0 | 0.02 | 0 | 0 | 0 | 0 | 0 | 0 | 0 | 0 |

| Gene | MORC3 | MORF4L2 | MRPL15 | MTREX | MYL6 | MYL6P5 | NCBP1 | NCBP2 | NCKIPSD | NDUFB5 |
|---|---|---|---|---|---|---|---|---|---|---|
| rf0 | 0.001685 | 0 | 0.000929 | 0 | 0.002715 | 0.000671 | 0.000311 | 0.000276 | 0.009356 | 0.004272 |
| rf1 | 0.001075 | 0.000377 | 7.60E−05 | 0.00481 | 0.000684 | 2.71E−18 | 0 | 0 | 0 | 3.07E−17 |
| rf2 | 0 | 0 | 0 | 0 | 0.004002 | 2.16E−18 | 0.001538 | 0.000942 | 0.003129 | 0.002065 |
| rf3 | 0.001612 | 0.000476 | 0 | 4.70E−18 | 0.000608 | 0 | 0.001501 | 0.001142 | 0 | 4.80E−18 |
| rf4 | 0.000657 | 0 | 0.005403 | 0 | 0.004656 | 0.001446 | 0.006742 | 0.000653 | 1.56E−18 | 0.001917 |
| rf5 | 0.005218 | 4.95E−19 | 0.00465 | 0 | 0.003855 | 0 | 0.003245 | 0.000285 | 0.003263 | 0.003634 |
| rf6 | 0.001108 | 0.002976 | 0.002511 | 5.37E−18 | 6.46E−19 | 0 | 0.007559 | 0 | 0.000324 | 0.002747 |
| rf7 | 0.000358 | 0.000343 | 0 | 0.004467 | 3.87E−18 | 0.001618 | 0 | 0.000945 | 0 | 0.001023 |
| rf8 | 0.001531 | 0.000719 | 0 | 0.004196 | 0.000945 | 0 | 0.003097 | 0.00035 | 0 | 0.000349 |
| rf9 | 4.55E−18 | 0 | 0 | 0.002716 | 0 | 0.003822 | 0.001174 | 0.007132 | 0.002607 | 0.000885 |
| rf10 | 0 | 0 | 0 | 0.000733 | 0.000888 | 0 | 0 | 1.01E−18 | 0 | 0.001252 |
| rf11 | 1.05E−17 | 0.000682 | 0.002603 | 0 | 0.001191 | 0 | 3.49E−19 | 0 | 1.24E−18 | 0 |
| rf12 | 0.000612 | 0.000429 | 0.004435 | 0.002729 | 0.000675 | 0.000374 | 0 | 0.001112 | 0.003423 | 1.09E−18 |
| rf13 | 0.00263 | 7.81E−19 | 0.001253 | 0 | 5.38E−18 | 0.000298 | 0.002304 | 0 | 0.001591 | 0.001937 |
| rf14 | 0.001685 | 0 | 0.000929 | 0 | 0.002715 | 0.000671 | 0.000311 | 0.000276 | 0.009356 | 0.004272 |
| rf15 | 0.001075 | 0.000377 | 7.60E−05 | 0.00481 | 0.000684 | 2.71E−18 | 0 | 0 | 0 | 3.07E−17 |
| rf16 | 0 | 0 | 0 | 0 | 0.004002 | 2.16E−18 | 0.001538 | 0.000942 | 0.003129 | 0.002065 |
| rf17 | 0.001612 | 0.000476 | 0 | 4.70E−18 | 0.000608 | 0 | 0.001501 | 0.001142 | 0 | 4.80E−18 |
| rf18 | 0.000657 | 0 | 0.005403 | 0 | 0.004656 | 0.001446 | 0.006742 | 0.000653 | 1.56E−18 | 0.001917 |
| rf19 | 0.005218 | 4.95E−19 | 0.00465 | 0 | 0.003855 | 0 | 0.003245 | 0.000285 | 0.003263 | 0.003634 |
| rf20 | 0.001108 | 0.002976 | 0.002511 | 5.37E−18 | 6.46E−19 | 0 | 0.007559 | 0 | 0.000324 | 0.002747 |
| rf21 | 0.000358 | 0.000343 | 0 | 0.004467 | 3.87E−18 | 0.001618 | 0 | 0.000945 | 0 | 0.001023 |
| rf22 | 0.001531 | 0.000719 | 0 | 0.004196 | 0.000945 | 0 | 0.003097 | 0.00035 | 0 | 0.000349 |
| rf23 | 4.55E−18 | 0 | 0 | 0.002716 | 0 | 0.003822 | 0.001174 | 0.007132 | 0.002607 | 0.000885 |
| rf24 | 0 | 0 | 0 | 0.000733 | 0.000888 | 0 | 0 | 1.01E−18 | 0 | 0.001252 |
| rf25 | 1.05E−17 | 0.000682 | 0.002603 | 0 | 0.001191 | 0 | 3.49E−19 | 0 | 1.24E−18 | 0 |
| rf26 | 0.000612 | 0.000429 | 0.004435 | 0.002729 | 0.000675 | 0.000374 | 0 | 0.001112 | 0.003423 | 1.09E−18 |
| rf27 | 0.00263 | 7.81E−19 | 0.001253 | 0 | 5.38E−18 | 0.000298 | 0.002304 | 0 | 0.001591 | 0.001937 |
| extraTrees0 | 0.001198 | 0.000492 | 0.004091 | 0.00058 | 0.003334 | 0 | 0.002833 | 0.002805 | 5.48E−05 | 0.003025 |
| extraTrees1 | 0 | 0.002442 | 0.002408 | 0.000423 | 0.002583 | 0.002328 | 0.000546 | 0.000342 | 0.002927 | 5.99E−05 |
| extraTrees2 | 0.002876 | 0 | 0.000758 | 0.000525 | 0.002578 | 0.002133 | 0.000492 | 0.001849 | 0.001854 | 0.003685 |
| extraTrees3 | 0.0003 | 0.002392 | 0.001928 | 0.003979 | 0.002128 | 0.001149 | 0.000584 | 0.002782 | 0.007806 | 0.000839 |
| extraTrees4 | 0.002693 | 0.000746 | 0.00176 | 0.00052 | 0.002078 | 0.003047 | 0.000762 | 0.000535 | 0 | 0.000469 |
| extraTrees5 | 0.000238 | 0.001452 | 0.005189 | 0.002832 | 0.002532 | 0.000532 | 0.000707 | 0.000776 | 0.004435 | 0.001841 |
| extraTrees6 | 0.001999 | 0 | 0.002514 | 0.001308 | 0.000476 | 1.50E−18 | 0.000937 | 0.001886 | 0.008375 | 0.000301 |
| extraTrees7 | 0.000416 | 0.000387 | 0.000996 | 1.26E−18 | 0.001807 | 0.001286 | 0.001983 | 0.003164 | 0.005034 | 0.001046 |
| extraTrees8 | 0.001668 | 0.000915 | 0.006595 | 0.001339 | 0.001251 | 0.00131 | 0.000863 | 0.000826 | 0.003787 | 0.001627 |
| extraTrees9 | 0.000825 | 0.000434 | 0.001493 | 0.001826 | 0.001215 | 0.000535 | 0.000492 | 0.002093 | 0.006089 | 0.002358 |
| extraTrees10 | 0.001201 | 0.000488 | 0.00191 | 0 | 0.001332 | 0.000998 | 0.002864 | 0.003393 | 0.003263 | 0.000515 |
| extraTrees11 | 0.000924 | 0.000999 | 0.000537 | 0.000238 | 0.00179 | 0.000689 | 2.05E−19 | 0.000459 | 0.001459 | 0.002489 |
| extraTrees12 | 0.000545 | 0.001804 | 0.003009 | 0.003718 | 0.004076 | 0.00028 | 0.00047 | 0.001874 | 0.006093 | 0.000676 |
| extraTrees13 | 0.000865 | 0.000427 | 0.002055 | 0.001429 | 0.000905 | 0.001868 | 0.00138 | 0.001709 | 0.002986 | 0.001565 |
| extraTrees14 | 0.001198 | 0.000492 | 0.004091 | 0.00058 | 0.003334 | 0 | 0.002833 | 0.002805 | 5.48E−05 | 0.003025 |
| extraTrees15 | 0 | 0.002442 | 0.002408 | 0.000423 | 0.002583 | 0.002328 | 0.000546 | 0.000342 | 0.002927 | 5.99E−05 |
| extraTrees16 | 0.002876 | 0 | 0.000758 | 0.000525 | 0.002578 | 0.002133 | 0.000492 | 0.001849 | 0.001854 | 0.003685 |
| extraTrees17 | 0.0003 | 0.002392 | 0.001928 | 0.003979 | 0.002128 | 0.001149 | 0.000584 | 0.002782 | 0.007806 | 0.000839 |
| extraTrees18 | 0.002693 | 0.000746 | 0.00176 | 0.00052 | 0.002078 | 0.003047 | 0.000762 | 0.000535 | 0 | 0.000469 |
| extraTrees19 | 0.000238 | 0.001452 | 0.005189 | 0.002832 | 0.002532 | 0.000532 | 0.000707 | 0.000776 | 0.004435 | 0.001841 |
| extraTrees20 | 0.001999 | 0 | 0.002514 | 0.001308 | 0.000476 | 1.50E−18 | 0.000937 | 0.001886 | 0.008375 | 0.000301 |
| extraTrees21 | 0.000416 | 0.000387 | 0.000996 | 1.26E−18 | 0.001807 | 0.001286 | 0.001983 | 0.003164 | 0.005034 | 0.001046 |
| extraTrees22 | 0.001668 | 0.000915 | 0.006595 | 0.001339 | 0.001251 | 0.00131 | 0.000863 | 0.000826 | 0.003787 | 0.001627 |
| extraTrees23 | 0.000825 | 0.000434 | 0.001493 | 0.001826 | 0.001215 | 0.000535 | 0.000492 | 0.002093 | 0.006089 | 0.002358 |
| extraTrees24 | 0.001201 | 0.000488 | 0.00191 | 0 | 0.001332 | 0.000998 | 0.002864 | 0.003393 | 0.003263 | 0.000515 |
| extraTrees25 | 0.000924 | 0.000999 | 0.000537 | 0.000238 | 0.00179 | 0.000689 | 2.05E−19 | 0.000459 | 0.001459 | 0.002489 |
| extraTrees26 | 0.000545 | 0.001804 | 0.003009 | 0.003718 | 0.004076 | 0.00028 | 0.00047 | 0.001874 | 0.006093 | 0.000676 |
| extraTrees27 | 0.000865 | 0.000427 | 0.002055 | 0.001429 | 0.000905 | 0.001868 | 0.00138 | 0.001709 | 0.002986 | 0.001565 |
| gbc0 | 0.00491 | 9.23E−05 | 0 | 0 | 0.000186 | 0 | 0 | 0 | 0.002292 | 0 |
| gbc1 | 0 | 1.51E−06 | 8.82E−05 | 8.63E−06 | 0.040488 | 0.00968 | 0.006945 | 0.001136 | 0 | 0 |
| gbc2 | 2.34E−05 | 1.65E−05 | 0 | 0 | 0 | 0 | 0.001094 | 0 | 0.003587 | 0 |
| gbc3 | 5.51E−05 | 0 | 0 | 0 | 0 | 0.000659 | 3.76E−05 | 0.003336 | 0.000154 | 0 |
| gbc4 | 0.000206 | 0 | 5.86E−05 | 0.000621 | 0 | 2.50E−05 | 0 | 0 | 0 | 0.000239 |
| gbc5 | 3.45E−06 | 0 | 0.000582 | 0 | 0.000379 | 0 | 0 | 0 | 8.02E−05 | 0 |
| gbc6 | 0 | 0 | 5.36E−05 | 0 | 3.45E−05 | 0.000784 | 0 | 8.84E−06 | 0.000706 | 0 |
| gbc7 | 0.002555 | 0 | 0.001185 | 0 | 0 | 0 | 0 | 4.58E−08 | 2.12E−06 | 0.008207 |
| gbc8 | 2.88E−05 | 1.07E−05 | 0 | 0 | 8.62E−05 | 0 | 2.73E−05 | 4.23E−05 | 2.32E−06 | 0 |
| gbc9 | 0 | 0 | 0.000222 | 0.000825 | 0 | 8.07E−07 | 0 | 0.002816 | 0 | 0 |
| gbc10 | 0 | 0 | 1.17E−05 | 0 | 0 | 0.003551 | 0 | 0 | 0 | 0 |
| gbc11 | 0 | 0 | 0 | 0 | 0.002213 | 7.78E−06 | 1.29E−06 | 0 | 0 | 0 |
| gbc12 | 0 | 0 | 0.000728 | 0 | 1.05E−09 | 0 | 0 | 0 | 0.000325 | 0 |
| gbc13 | 0 | 0 | 3.89E−05 | 0.000944 | 0.000157 | 0 | 0 | 0 | 1.17E−05 | 0 |
| gbc14 | 0.00491 | 9.23E−05 | 0 | 0 | 0.000186 | 0 | 0 | 0 | 0.002292 | 0 |
| gbc15 | 0 | 1.51E−06 | 8.82E−05 | 8.63E−06 | 0.040488 | 0.00968 | 0.006945 | 0.001136 | 0 | 0 |
| gbc16 | 2.34E−05 | 1.65E−05 | 0 | 0 | 0 | 0 | 0.001094 | 0 | 0.003587 | 0 |
| gbc17 | 5.51E−05 | 0 | 0 | 0 | 0 | 0.000659 | 3.76E−05 | 0.003336 | 0.000154 | 0 |
| gbc18 | 0.000206 | 0 | 5.86E−05 | 0.000621 | 0 | 2.50E−05 | 0 | 0 | 0 | 0.000239 |
| gbc19 | 3.45E−06 | 0 | 0.000582 | 0 | 0.000379 | 0 | 0 | 0 | 8.02E−05 | 0 |
| gbc20 | 0 | 0 | 5.36E−05 | 0 | 3.45E−05 | 0.000784 | 0 | 8.84E−06 | 0.000706 | 0 |
| gbc21 | 0.002555 | 0 | 0.001185 | 0 | 0 | 0 | 0 | 4.58E−08 | 2.12E−06 | 0.008207 |
| gbc22 | 2.88E−05 | 1.07E−05 | 0 | 0 | 8.62E−05 | 0 | 2.73E−05 | 4.23E−05 | 2.32E−06 | 0 |
| gbc23 | 0 | 0 | 0.000222 | 0.000825 | 0 | 8.07E−07 | 0 | 0.002816 | 0 | 0 |
| gbc24 | 0 | 0 | 1.17E−05 | 0 | 0 | 0.003551 | 0 | 0 | 0 | 0 |
| gbc25 | 0 | 0 | 0 | 0 | 0.002213 | 7.78E−06 | 1.29E−06 | 0 | 0 | 0 |
| gbc26 | 0 | 0 | 0.000728 | 0 | 1.05E−09 | 0 | 0 | 0 | 0.000325 | 0 |
| gbc27 | 0 | 0 | 3.89E−05 | 0.000944 | 0.000157 | 0 | 0 | 0 | 1.17E−05 | 0 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | NFE2L2 | NINJ1 | NMD3 | NMI | NPAT | NSUN6 | NUTF2 | OGFRL1 | ORAI2 | ORC5 |
|---|---|---|---|---|---|---|---|---|---|---|
| rf0 | 0.008551 | 0.000642 | 0.010739 | 0.004971 | 0 | 0.003225 | 0.00184 | 0.00035 | 0 | 0.002152 |
| rf1 | 0.000785 | 0.000288 | 0.009904 | 0.005829 | 0.006377 | 0.000661 | 0 | 0.00034 | 0.000762 | 0.007791 |
| rf2 | 0.00175 | 0.000961 | 0.002628 | 0.001131 | 0.005296 | 0.00085 | 0.005029 | 0.017776 | 0 | 0.000384 |
| rf3 | 0 | 0.002509 | 0.007818 | 4.17E−18 | 0.008378 | 0.004797 | 0.002276 | 0.009042 | 0.000719 | 0.004259 |
| rf4 | 0 | 0 | 0.004529 | 0.004812 | 0 | 0.004998 | 0.000396 | 0.005343 | 0 | 0.000712 |
| rf5 | 0.000673 | 0 | 0.004686 | 0.000744 | 0.006255 | 0.006296 | 0.00392 | 0.000312 | 0 | 0.000745 |
| rf6 | 0.000298 | 0.002194 | 0.004076 | 0 | 0.012865 | 0.003051 | 0.002033 | 0.000692 | 0.004937 | 0.003137 |
| rf7 | 0.00218 | 0.004787 | 0.011886 | 0.000358 | 0.001176 | 0.003748 | 0.011067 | 0.003498 | 0 | 0.002818 |
| rf8 | 0 | 0 | 0.010554 | 0.000304 | 0.003178 | 0.0082 | 0.005776 | 0.011508 | 0 | 0.008608 |
| rf9 | 0.008459 | 0.005509 | 0.01666 | 0.000313 | 0.005341 | 0.011208 | 0.006091 | 0.006691 | 0 | 0.003903 |
| rf10 | 0.00456 | 0.003882 | 0.009718 | 0.000845 | 0 | 0 | 0.003522 | 0.006987 | 0.005687 | 0.002472 |
| rf11 | 0.001621 | 0.00034 | 0.005004 | 0.003662 | 0.004776 | 0.005764 | 0.0054 | 0.002264 | 0.002047 | 0.000869 |
| rf12 | 0.002534 | 0.002253 | 0.007981 | 0.000393 | 0.005562 | 0.005272 | 0 | 0.008798 | 0 | 0.000356 |
| rf13 | 0.002036 | 0.00176 | 0.010435 | 4.15E−18 | 0.001018 | 0.004032 | 0.000802 | 0.001536 | 0.002669 | 0.001606 |
| rf14 | 0.008551 | 0.000642 | 0.010739 | 0.004971 | 0 | 0.003225 | 0.00184 | 0.00035 | 0 | 0.002152 |
| rf15 | 0.000785 | 0.000288 | 0.009904 | 0.005829 | 0.006377 | 0.000661 | 0 | 0.00034 | 0.000762 | 0.007791 |
| rf16 | 0.00175 | 0.000961 | 0.002628 | 0.001131 | 0.005296 | 0.00085 | 0.005029 | 0.017776 | 0 | 0.000384 |
| rf17 | 0 | 0.002509 | 0.007818 | 4.17E−18 | 0.008378 | 0.004797 | 0.002276 | 0.009042 | 0.000719 | 0.004259 |
| rf18 | 0 | 0 | 0.004529 | 0.004812 | 0 | 0.004998 | 0.000396 | 0.005343 | 0 | 0.000712 |
| rf19 | 0.000673 | 0 | 0.004686 | 0.000744 | 0.006255 | 0.006296 | 0.00392 | 0.000312 | 0 | 0.000745 |
| rf20 | 0.000298 | 0.002194 | 0.004076 | 0 | 0.012865 | 0.003051 | 0.002033 | 0.000692 | 0.004937 | 0.003137 |
| rf21 | 0.00218 | 0.004787 | 0.011886 | 0.000358 | 0.001176 | 0.003748 | 0.011067 | 0.003498 | 0 | 0.002818 |
| rf22 | 0 | 0 | 0.010554 | 0.000304 | 0.003178 | 0.0082 | 0.005776 | 0.011508 | 0 | 0.008608 |
| rf23 | 0.008459 | 0.005509 | 0.01666 | 0.000313 | 0.005341 | 0.011208 | 0.006091 | 0.006691 | 0 | 0.003903 |
| rf24 | 0.00456 | 0.003882 | 0.009718 | 0.000845 | 0 | 0 | 0.003522 | 0.006987 | 0.005687 | 0.002472 |
| rf25 | 0.001621 | 0.00034 | 0.005004 | 0.003662 | 0.004776 | 0.005764 | 0.0054 | 0.002264 | 0.002047 | 0.000869 |
| rf26 | 0.002534 | 0.002253 | 0.007981 | 0.000393 | 0.005562 | 0.005272 | 0 | 0.008798 | 0 | 0.000356 |
| rf27 | 0.002036 | 0.00176 | 0.010435 | 4.15E−18 | 0.001018 | 0.004032 | 0.000802 | 0.001536 | 0.002669 | 0.001606 |
| extraTrees0 | 0.001546 | 0.002908 | 0.002049 | 0.000677 | 0.000942 | 0.000861 | 0.005123 | 0.001007 | 0.00091 | 0.004838 |
| extraTrees1 | 0.003898 | 0.001135 | 0.000726 | 0.001228 | 0.000686 | 0.00426 | 0.002247 | 0.001701 | 0.00131 | 0.004682 |
| extraTrees2 | 7.01E−19 | 0.003029 | 0.009165 | 0.002524 | 0.003951 | 0.005195 | 0.004319 | 0.002517 | 0.005315 | 0.005084 |
| extraTrees3 | 0.001623 | 0.005374 | 0.007589 | 2.44E−18 | 0.004875 | 0.001554 | 0.00588 | 0.003087 | 0.004168 | 0.001645 |
| extraTrees4 | 0.001158 | 0.002135 | 0.002646 | 0.001296 | 0.002911 | 0.002079 | 0.004086 | 0.001199 | 0.001996 | 0.000754 |
| extraTrees5 | 0.002775 | 0.001061 | 0.001817 | 0.002223 | 0.004393 | 0.003258 | 0.00409 | 0.003105 | 0.002878 | 0.001787 |
| extraTrees6 | 0.002216 | 0.001064 | 0.004651 | 6.28E−05 | 0.004228 | 0.002119 | 0.007598 | 0.001478 | 0.001985 | 0.002844 |
| extraTrees7 | 0.003752 | 0.001613 | 0.005056 | 0 | 0.00294 | 0.002575 | 3.26E−18 | 0.001113 | 0.000293 | 0.004715 |
| extraTrees8 | 0.000737 | 0.00236 | 0.007086 | 0.001549 | 0.000746 | 0.002059 | 0.004726 | 0.001077 | 0.005481 | 0.002914 |
| extraTrees9 | 0.001544 | 0.005875 | 0.003008 | 0.000238 | 0.003226 | 0.005471 | 0.004297 | 0.003702 | 0.001702 | 0.005375 |
| extraTrees10 | 0.000978 | 0.003055 | 0.007414 | 0.001123 | 0.003474 | 0.001082 | 0.003707 | 0.006703 | 0.001712 | 0.005676 |
| extraTrees11 | 0.000492 | 0.003024 | 0.003116 | 0.00028 | 0.004972 | 0.003716 | 0.005198 | 0.003891 | 1.50E−05 | 0.002992 |
| extraTrees12 | 0.000818 | 0.000918 | 0.005787 | 0.000238 | 0.004683 | 0.001175 | 0.001623 | 0.005264 | 0.004014 | 0.001587 |
| extraTrees13 | 0.001321 | 0.002781 | 0.001434 | 0.001259 | 0.001139 | 0.003853 | 0.002203 | 0.00062 | 0.002498 | 0.004418 |
| extraTrees14 | 0.001546 | 0.002908 | 0.002049 | 0.000677 | 0.000942 | 0.000861 | 0.005123 | 0.001007 | 0.00091 | 0.004838 |
| extraTrees15 | 0.003898 | 0.001135 | 0.000726 | 0.001228 | 0.000686 | 0.00426 | 0.002247 | 0.001701 | 0.00131 | 0.004682 |
| extraTrees16 | 7.01E−19 | 0.003029 | 0.009165 | 0.002524 | 0.003951 | 0.005195 | 0.004319 | 0.002517 | 0.005315 | 0.005084 |
| extraTrees17 | 0.001623 | 0.005374 | 0.007589 | 2.44E−18 | 0.004875 | 0.001554 | 0.00588 | 0.003087 | 0.004168 | 0.001645 |
| extraTrees18 | 0.001158 | 0.002135 | 0.002646 | 0.001296 | 0.002911 | 0.002079 | 0.004086 | 0.001199 | 0.001996 | 0.000754 |
| extraTrees19 | 0.002775 | 0.001061 | 0.001817 | 0.002223 | 0.004393 | 0.003258 | 0.00409 | 0.003105 | 0.002878 | 0.001787 |
| extraTrees20 | 0.002216 | 0.001064 | 0.004651 | 6.28E−05 | 0.004228 | 0.002119 | 0.007598 | 0.001478 | 0.001985 | 0.002844 |
| extraTrees21 | 0.003752 | 0.001613 | 0.005056 | 0 | 0.00294 | 0.002575 | 3.26E−18 | 0.001113 | 0.000293 | 0.004715 |
| extraTrees22 | 0.000737 | 0.00236 | 0.007086 | 0.001549 | 0.000746 | 0.002059 | 0.004726 | 0.001077 | 0.005481 | 0.002914 |
| extraTrees23 | 0.001544 | 0.005875 | 0.003008 | 0.000238 | 0.003226 | 0.005471 | 0.004297 | 0.003702 | 0.001702 | 0.005375 |
| extraTrees24 | 0.000978 | 0.003055 | 0.007414 | 0.001123 | 0.003474 | 0.001082 | 0.003707 | 0.006703 | 0.001712 | 0.005676 |
| extraTrees25 | 0.000492 | 0.003024 | 0.003116 | 0.00028 | 0.004972 | 0.003716 | 0.005198 | 0.003891 | 1.50E−05 | 0.002992 |
| extraTrees26 | 0.000818 | 0.000918 | 0.005787 | 0.000238 | 0.004683 | 0.001175 | 0.001623 | 0.005264 | 0.004014 | 0.001587 |
| extraTrees27 | 0.001321 | 0.002781 | 0.001434 | 0.001259 | 0.001139 | 0.003853 | 0.002203 | 0.00062 | 0.002498 | 0.004418 |
| gbc0 | 0 | 0 | 0 | 0 | 0 | 0.000211 | 0 | 0.001923 | 0.001651 | 0.002554 |
| gbc1 | 0 | 0.021857 | 0 | 0 | 0.00252 | 0.000922 | 2.77E−06 | 0.000108 | 0 | 0 |
| gbc2 | 0.000136 | 2.27E−05 | 0.000828 | 0.000126 | 0 | 0.000371 | 0 | 0.027939 | 0 | 0 |
| gbc3 | 0 | 0.000143 | 0.012889 | 8.40E−07 | 0 | 0.005521 | 1.75E−05 | 0.007645 | 0 | 0 |
| gbc4 | 0 | 0 | 2.10E−05 | 0 | 0.013696 | 0.001403 | 0 | 0.000121 | 0 | 0.01046 |
| gbc5 | 0.003351 | 0 | 0.000218 | 0 | 0 | 0.005119 | 3.23E−06 | 0.006656 | 0 | 0 |
| gbc6 | 0 | 2.53E−05 | 0 | 0 | 0.00509 | 0.001538 | 0.020191 | 0.026896 | 0 | 0.001806 |
| gbc7 | 0 | 0 | 6.81E−05 | 0 | 3.87E−06 | 2.24E−05 | 0 | 0.005037 | 0 | 0 |
| gbc8 | 0.019335 | 0 | 0.000573 | 0 | 0 | 0.002227 | 0 | 0.011923 | 0.000727 | 0 |
| gbc9 | 4.57E−05 | 0 | 0.000606 | 0.000556 | 7.08E−05 | 0.005016 | 0 | 0.01566 | 0 | 0.006354 |
| gbc10 | 9.62E−06 | 0 | 0.000295 | 0 | 0 | 0.02423 | 0.032727 | 0.000614 | 0 | 0.001227 |
| gbc11 | 0 | 0 | 7.11E−05 | 0.000423 | 0.026225 | 3.69E−06 | 0.018877 | 0.004639 | 1.15E−06 | 0.000128 |
| gbc12 | 0.000242 | 3.49E−05 | 0 | 0 | 0 | 0 | 8.55E−06 | 7.36E−05 | 0.001105 | 0.000107 |
| gbc13 | 0.004608 | 0.005364 | 0.004139 | 0 | 0 | 8.90E−06 | 0.003241 | 0.018919 | 0 | 0 |
| gbc14 | 0 | 0 | 0 | 0 | 0 | 0.000211 | 0 | 0.001923 | 0.001651 | 0.002554 |
| gbc15 | 0 | 0.021857 | 0 | 0 | 0.00252 | 0.000922 | 2.77E−06 | 0.000108 | 0 | 0 |
| gbc16 | 0.000136 | 2.27E−05 | 0.000828 | 0.000126 | 0 | 0.000371 | 0 | 0.027939 | 0 | 0 |
| gbc17 | 0 | 0.000143 | 0.012889 | 8.40E−07 | 0 | 0.005521 | 1.75E−05 | 0.007645 | 0 | 0 |
| gbc18 | 0 | 0 | 2.10E−05 | 0 | 0.013696 | 0.001403 | 0 | 0.000121 | 0 | 0.01046 |
| gbc19 | 0.003351 | 0 | 0.000218 | 0 | 0 | 0.005119 | 3.23E−06 | 0.006656 | 0 | 0 |
| gbc20 | 0 | 2.53E−05 | 0 | 0 | 0.00509 | 0.001538 | 0.020191 | 0.026896 | 0 | 0.001806 |
| gbc21 | 0 | 0 | 6.81E−05 | 0 | 3.87E−06 | 2.24E−05 | 0 | 0.005037 | 0 | 0 |
| gbc22 | 0.019335 | 0 | 0.000573 | 0 | 0 | 0.002227 | 0 | 0.011923 | 0.000727 | 0 |
| gbc23 | 4.57E−05 | 0 | 0.000606 | 0.000556 | 7.08E−05 | 0.005016 | 0 | 0.01566 | 0 | 0.006354 |
| gbc24 | 9.62E−06 | 0 | 0.000295 | 0 | 0 | 0.02423 | 0.032727 | 0.000614 | 0 | 0.001227 |
| gbc25 | 0 | 0 | 7.11E−05 | 0.000423 | 0.026225 | 3.69E−06 | 0.018877 | 0.004639 | 1.15E−06 | 0.000128 |
| gbc26 | 0.000242 | 3.49E−05 | 0 | 0 | 0 | 0 | 8.55E−06 | 7.36E−05 | 0.001105 | 0.000107 |
| gbc27 | 0.004608 | 0.005364 | 0.004139 | 0 | 0 | 8.90E−06 | 0.003241 | 0.018919 | 0 | 0 |
| ada0 | 0 | 0 | 0 | 0 | 0 | 0 | 0 | 0.02 | 0 | 0 |
| ada1 | 0 | 0 | 0 | 0 | 0 | 0 | 0 | 0.02 | 0 | 0 |
| ada2 | 0 | 0 | 0 | 0 | 0 | 0 | 0 | 0.02 | 0 | 0 |
| ada3 | 0 | 0 | 0 | 0 | 0 | 0 | 0 | 0.02 | 0 | 0 |
| ada4 | 0 | 0 | 0 | 0 | 0 | 0 | 0 | 0.02 | 0 | 0 |
| ada5 | 0 | 0 | 0 | 0 | 0 | 0 | 0 | 0.02 | 0 | 0 |
| ada6 | 0 | 0 | 0 | 0 | 0 | 0 | 0 | 0.02 | 0 | 0 |
| ada7 | 0 | 0 | 0 | 0 | 0 | 0 | 0 | 0.02 | 0 | 0 |
| ada8 | 0 | 0 | 0 | 0 | 0 | 0 | 0 | 0.02 | 0 | 0 |
| ada9 | 0 | 0 | 0 | 0 | 0 | 0 | 0 | 0.02 | 0 | 0 |
| ada10 | 0 | 0 | 0 | 0 | 0 | 0 | 0 | 0.02 | 0 | 0 |
| ada11 | 0 | 0 | 0 | 0 | 0 | 0 | 0 | 0.02 | 0 | 0 |
| ada12 | 0 | 0 | 0 | 0 | 0 | 0 | 0 | 0.02 | 0 | 0 |
| ada13 | 0 | 0 | 0 | 0 | 0 | 0 | 0 | 0.02 | 0 | 0 |
| ada14 | 0 | 0 | 0 | 0 | 0 | 0 | 0 | 0.02 | 0 | 0 |
| ada15 | 0 | 0 | 0 | 0 | 0 | 0 | 0 | 0.02 | 0 | 0 |
| ada16 | 0 | 0 | 0 | 0 | 0 | 0 | 0 | 0.02 | 0 | 0 |
| ada17 | 0 | 0 | 0 | 0 | 0 | 0 | 0 | 0.02 | 0 | 0 |
| ada18 | 0 | 0 | 0 | 0 | 0 | 0 | 0 | 0.02 | 0 | 0 |
| ada19 | 0 | 0 | 0 | 0 | 0 | 0 | 0 | 0.02 | 0 | 0 |
| ada20 | 0 | 0 | 0 | 0 | 0 | 0 | 0 | 0.02 | 0 | 0 |
| ada21 | 0 | 0 | 0 | 0 | 0 | 0 | 0 | 0.02 | 0 | 0 |
| ada22 | 0 | 0 | 0 | 0 | 0 | 0 | 0 | 0.02 | 0 | 0 |
| ada23 | 0 | 0 | 0 | 0 | 0 | 0 | 0 | 0.02 | 0 | 0 |
| ada24 | 0 | 0 | 0 | 0 | 0 | 0 | 0 | 0.02 | 0 | 0 |
| ada25 | 0 | 0 | 0 | 0 | 0 | 0 | 0 | 0.02 | 0 | 0 |
| ada26 | 0 | 0 | 0 | 0 | 0 | 0 | 0 | 0.02 | 0 | 0 |
| ada27 | 0 | 0 | 0 | 0 | 0 | 0 | 0 | 0.02 | 0 | 0 |

| Gene | OSBPL3 | P2RX1 | P2RY10 | PACSIN2 | PAFAH1B1 | PAIP1 | PAQR7 | PCSK6 | PDS5B | PEX3 |
|---|---|---|---|---|---|---|---|---|---|---|
| rf0 | 0.001239 | 0 | 0 | 0 | 4.31E−19 | 0 | 0.002865 | 0.002729 | 0.001694 | 0.00067 |
| rf1 | 0.003548 | 0.006107 | 0.00097 | 0.000461 | 0.004999 | 0.001 | 0.000856 | 6.41E−18 | 4.10E−18 | 0.002137 |
| rf2 | 0.000442 | 0.00429 | 0.007908 | 2.18E−17 | 0 | 0.008235 | 0.001347 | 0 | 0.001452 | 0 |
| rf3 | 0.002875 | 7.51E−19 | 0.005042 | 0.003396 | 0 | 0 | 1.96E−17 | 0.002727 | 0.001551 | 0.001388 |
| rf4 | 4.90E−19 | 0.000669 | 0.004731 | 2.25E−18 | 0.004238 | 0.005896 | 0 | 0 | 0.000288 | 0 |
| rf5 | 0.002601 | 4.74E−05 | 0.00354 | 0 | 0.001042 | 0.002542 | 0.001653 | 0.009115 | 0.005594 | 0.000605 |
| rf6 | 0.001358 | 0.00272 | 0.000635 | 0.00704 | 0 | 0.003079 | 0.00105 | 0.000699 | 0 | 4.64E−18 |
| rf7 | 0 | 0.000448 | 0.000652 | 0.001559 | 0.001289 | 0 | 0.002545 | 1.49E−18 | 0 | 0.004739 |
| rf8 | 0.003001 | 0 | 7.26E−19 | 0.000399 | 0.001358 | 0.003021 | 0.001071 | 0.002602 | 0.003682 | 0.004447 |
| rf9 | 0.000977 | 0 | 0.002336 | 0.00191 | 0 | 0.002317 | 0.000485 | 0 | 0.000951 | 0.001429 |
| rf10 | 0.000988 | 0 | 0 | 4.46E−18 | 0.004168 | 0.000424 | 0 | 0 | 0.000383 | 0 |
| rf11 | 0 | 0 | 0 | 0.002906 | 0.00452 | 0.00041 | 0.001246 | 0 | 0.005635 | 0 |
| rf12 | 0.004681 | 0 | 0.001951 | 0.008952 | 0 | 0.001548 | 0 | 0 | 0 | 0.004285 |
| rf13 | 0.004503 | 0 | 0.004578 | 0.000712 | 0 | 0.004339 | 0.000375 | 0.001053 | 0 | 5.25E−19 |
| rf14 | 0.001239 | 0 | 0 | 0 | 4.31E−19 | 0 | 0.002865 | 0.002729 | 0.001694 | 0.00067 |
| rf15 | 0.003548 | 0.006107 | 0.00097 | 0.000461 | 0.004999 | 0.001 | 0.000856 | 6.41E−18 | 4.10E−18 | 0.002137 |
| rf16 | 0.000442 | 0.00429 | 0.007908 | 2.18E−17 | 0 | 0.008235 | 0.001347 | 0 | 0.001452 | 0 |
| rf17 | 0.002875 | 7.51E−19 | 0.005042 | 0.003396 | 0 | 0 | 1.96E−17 | 0.002727 | 0.001551 | 0.001388 |
| rf18 | 4.90E−19 | 0.000669 | 0.004731 | 2.25E−18 | 0.004238 | 0.005896 | 0 | 0 | 0.000288 | 0 |
| rf19 | 0.002601 | 4.74E−05 | 0.00354 | 0 | 0.001042 | 0.002542 | 0.001653 | 0.009115 | 0.005594 | 0.000605 |
| rf20 | 0.001358 | 0.00272 | 0.000635 | 0.00704 | 0 | 0.003079 | 0.00105 | 0.000699 | 0 | 4.64E−18 |
| rf21 | 0 | 0.000448 | 0.000652 | 0.001559 | 0.001289 | 0 | 0.002545 | 1.49E−18 | 0 | 0.004739 |
| rf22 | 0.003001 | 0 | 7.26E−19 | 0.000399 | 0.001358 | 0.003021 | 0.001071 | 0.002602 | 0.003682 | 0.004447 |
| rf23 | 0.000977 | 0 | 0.002336 | 0.00191 | 0 | 0.002317 | 0.000485 | 0 | 0.000951 | 0.001429 |
| rf24 | 0.000988 | 0 | 0 | 4.46E−18 | 0.004168 | 0.000424 | 0 | 0 | 0.000383 | 0 |
| rf25 | 0 | 0 | 0 | 0.002906 | 0.00452 | 0.00041 | 0.001246 | 0 | 0.005635 | 0 |
| rf26 | 0.004681 | 0 | 0.001951 | 0.008952 | 0 | 0.001548 | 0 | 0 | 0 | 0.004285 |
| rf27 | 0.004503 | 0 | 0.004578 | 0.000712 | 0 | 0.004339 | 0.000375 | 0.001053 | 0 | 5.25E−19 |
| extraTrees0 | 0.001679 | 0.000476 | 0.000524 | 0.000562 | 0.00274 | 0.000772 | 0 | 0.000359 | 0.003139 | 0.004348 |
| extraTrees1 | 0.001274 | 0.000683 | 0.00442 | 1.41E−18 | 0.002972 | 0.000381 | 0 | 0.002353 | 0.001827 | 0.001579 |
| extraTrees2 | 0.001253 | 0.000301 | 0.004459 | 0.002583 | 0.001141 | 0.001168 | 0.001484 | 0.00076 | 0.00028 | 0.001948 |
| extraTrees3 | 0.001198 | 0.001183 | 0.001362 | 0.000238 | 0.002109 | 0 | 0.000985 | 0.000786 | 0.00314 | 0.000679 |
| extraTrees4 | 0.000378 | 0.001929 | 0.003276 | 0.000938 | 0.00062 | 0.001933 | 0.002362 | 0.001188 | 0.000548 | 0.003509 |
| extraTrees5 | 0.001472 | 0.00047 | 0.006177 | 0.000373 | 0.000994 | 0.002419 | 0.003383 | 0.002865 | 0.000522 | 0.004378 |
| extraTrees6 | 0.002793 | 0.002267 | 0.001489 | 0.000831 | 0.000719 | 0.002188 | 0.000661 | 0.001607 | 0.000996 | 0.002818 |
| extraTrees7 | 0.001476 | 0.001824 | 0.010648 | 0.001555 | 0.004914 | 0.000269 | 0.000471 | 0.001052 | 3.28E−18 | 0.000584 |
| extraTrees8 | 0 | 0.001062 | 0.001833 | 0.001406 | 0.001618 | 0.000295 | 0.001781 | 0.002085 | 0.002188 | 0.004107 |
| extraTrees9 | 0.001058 | 0.000403 | 0.001102 | 0.000841 | 0.0031 | 2.05E−19 | 0 | 0.001856 | 0.000238 | 0.002576 |
| extraTrees10 | 0.001775 | 0.000238 | 0.002571 | 0.000625 | 0.004734 | 0.002974 | 0.001964 | 0.001595 | 0.002014 | 0.002273 |
| extraTrees11 | 0.000964 | 0.000373 | 0.004581 | 0.000814 | 0.001902 | 5.26E−19 | 0 | 0.003667 | 0.001411 | 0.001504 |
| extraTrees12 | 0 | 0.001384 | 0.000581 | 0.00136 | 0.003311 | 0.002581 | 0.000238 | 0.002958 | 0.001524 | 0.003497 |
| extraTrees13 | 9.73E−05 | 0.001496 | 0.002521 | 0.002227 | 0.002808 | 0.001285 | 0.001266 | 0.000639 | 0.002116 | 0.003498 |
| extraTrees14 | 0.001679 | 0.000476 | 0.000524 | 0.000562 | 0.00274 | 0.000772 | 0 | 0.000359 | 0.003139 | 0.004348 |
| extraTrees15 | 0.001274 | 0.000683 | 0.00442 | 1.41E−18 | 0.002972 | 0.000381 | 0 | 0.002353 | 0.001827 | 0.001579 |
| extraTrees16 | 0.001253 | 0.000301 | 0.004459 | 0.002583 | 0.001141 | 0.001168 | 0.001484 | 0.00076 | 0.00028 | 0.001948 |
| extraTrees17 | 0.001198 | 0.001183 | 0.001362 | 0.000238 | 0.002109 | 0 | 0.000985 | 0.000786 | 0.00314 | 0.000679 |
| extraTrees18 | 0.000378 | 0.001929 | 0.003276 | 0.000938 | 0.00062 | 0.001933 | 0.002362 | 0.001188 | 0.000548 | 0.003509 |
| extraTrees19 | 0.001472 | 0.00047 | 0.006177 | 0.000373 | 0.000994 | 0.002419 | 0.003383 | 0.002865 | 0.000522 | 0.004378 |
| extraTrees20 | 0.002793 | 0.002267 | 0.001489 | 0.000831 | 0.000719 | 0.002188 | 0.000661 | 0.001607 | 0.000996 | 0.002818 |
| extraTrees21 | 0.001476 | 0.001824 | 0.010648 | 0.001555 | 0.004914 | 0.000269 | 0.000471 | 0.001052 | 3.28E−18 | 0.000584 |
| extraTrees22 | 0 | 0.001062 | 0.001833 | 0.001406 | 0.001618 | 0.000295 | 0.001781 | 0.002085 | 0.002188 | 0.004107 |
| extraTrees23 | 0.001058 | 0.000403 | 0.001102 | 0.000841 | 0.0031 | 2.05E−19 | 0 | 0.001856 | 0.000238 | 0.002576 |
| extraTrees24 | 0.001775 | 0.000238 | 0.002571 | 0.000625 | 0.004734 | 0.002974 | 0.001964 | 0.001595 | 0.002014 | 0.002273 |
| extraTrees25 | 0.000964 | 0.000373 | 0.004581 | 0.000814 | 0.001902 | 5.26E−19 | 0 | 0.003667 | 0.001411 | 0.001504 |
| extraTrees26 | 0 | 0.001384 | 0.000581 | 0.00136 | 0.003311 | 0.002581 | 0.000238 | 0.002958 | 0.001524 | 0.003497 |
| extraTrees27 | 9.73E−05 | 0.001496 | 0.002521 | 0.002227 | 0.002808 | 0.001285 | 0.001266 | 0.000639 | 0.002116 | 0.003498 |
| gbc0 | 0 | 0 | 0.000101 | 0 | 0.02506 | 0 | 0.000148 | 0 | 0.000283 | 0.00013 |
| gbc1 | 0 | 0 | 2.46E−05 | 0 | 6.18E−05 | 0 | 0 | 0 | 0 | 0 |
| gbc2 | 0 | 3.65E−05 | 0 | 0.002945 | 0.00062 | 0 | 0 | 0 | 0 | 0.000957 |
| gbc3 | 0 | 0 | 0 | 0 | 0.000761 | 0.001257 | 0 | 0.00021 | 0.000424 | 2.78E−05 |
| gbc4 | 0 | 0 | 0 | 0.000864 | 0.004235 | 8.75E−05 | 1.91E−07 | 0.000179 | 0 | 0.006456 |
| gbc5 | 0.007405 | 0 | 0 | 0 | 0.000288 | 0.000447 | 0 | 0 | 1.18E−05 | 0 |
| gbc6 | 0 | 0.003192 | 4.45E−05 | 0 | 0.000154 | 0.005431 | 0.014239 | 0 | 0.0022 | 1.03E−05 |
| gbc7 | 1.75E−06 | 0.003262 | 0 | 0 | 0.003262 | 0 | 0.007093 | 0 | 0 | 0 |
| gbc8 | 0.00135 | 7.75E−05 | 0.000126 | 0.000312 | 0 | 1.90E−05 | 0 | 0 | 0 | 0 |
| gbc9 | 0 | 0 | 0 | 0 | 0.009734 | 0 | 0.008211 | 8.70E−06 | 0 | 4.83E−06 |
| gbc10 | 0.006462 | 0 | 0.001679 | 0 | 0.00022 | 2.91E−06 | 7.10E−05 | 0.001522 | 0.004913 | 4.08E−05 |
| gbc11 | 5.21E−05 | 0 | 0 | 2.26E−07 | 0.003256 | 0 | 0.014083 | 0 | 0.004495 | 0 |
| gbc12 | 0 | 0.001165 | 0 | 0 | 0.055719 | 0.000101 | 0 | 0 | 0 | 0 |
| gbc13 | 0 | 0 | 0 | 5.60E−06 | 0.001466 | 0 | 0.000895 | 0 | 0 | 4.25E−06 |
| gbc14 | 0 | 0 | 0.000101 | 0 | 0.02506 | 0 | 0.000148 | 0 | 0.000283 | 0.00013 |
| gbc15 | 0 | 0 | 2.46E−05 | 0 | 6.18E−05 | 0 | 0 | 0 | 0 | 0 |
| gbc16 | 0 | 3.65E−05 | 0 | 0.002945 | 0.00062 | 0 | 0 | 0 | 0 | 0.000957 |
| gbc17 | 0 | 0 | 0 | 0 | 0.000761 | 0.001257 | 0 | 0.00021 | 0.000424 | 2.78E−05 |
| gbc18 | 0 | 0 | 0 | 0.000864 | 0.004235 | 8.75E−05 | 1.91E−07 | 0.000179 | 0 | 0.006456 |
| gbc19 | 0.007405 | 0 | 0 | 0 | 0.000288 | 0.000447 | 0 | 0 | 1.18E−05 | 0 |
| gbc20 | 0 | 0.003192 | 4.45E−05 | 0 | 0.000154 | 0.005431 | 0.014239 | 0 | 0.0022 | 1.03E−05 |
| gbc21 | 1.75E−06 | 0.003262 | 0 | 0 | 0.003262 | 0 | 0.007093 | 0 | 0 | 0 |
| gbc22 | 0.00135 | 7.75E−05 | 0.000126 | 0.000312 | 0 | 1.90E−05 | 0 | 0 | 0 | 0 |
| gbc23 | 0 | 0 | 0 | 0 | 0.009734 | 0 | 0.008211 | 8.70E−06 | 0 | 4.83E−06 |
| gbc24 | 0.006462 | 0 | 0.001679 | 0 | 0.00022 | 2.91E−06 | 7.10E−05 | 0.001522 | 0.004913 | 4.08E−05 |
| gbc25 | 5.21E−05 | 0 | 0 | 2.26E−07 | 0.003256 | 0 | 0.014083 | 0 | 0.004495 | 0 |
| gbc26 | 0 | 0.001165 | 0 | 0 | 0.055719 | 0.000101 | 0 | 0 | 0 | 0 |
| gbc27 | 0 | 0 | 0 | 5.60E−06 | 0.001466 | 0 | 0.000895 | 0 | 0 | 4.25E−06 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | PGAM1 | PGD | PHF3 | PHF6 | PI4K2B | PI4KA | PIAS1 | PIGS | PIK3R1 | PLA2G4C |
|---|---|---|---|---|---|---|---|---|---|---|
| rf0 | 0.001134 | 0.000737 | 0.004315 | 0.002797 | 0.005707 | 0 | 0.003375 | 0 | 0.002982 | 9.58E−05 |
| rf1 | 0 | 0.005724 | 0.00431 | 0.009576 | 0.003918 | 3.27E−18 | 0 | 0.001857 | 0.000288 | 0.005228 |
| rf2 | 0 | 0.004923 | 0 | 0.003785 | 0 | 0.00165 | 0 | 7.55E−19 | 0.002045 | 0.001984 |
| rf3 | 0.00036 | 0 | 0.003583 | 0.000339 | 0 | 0.001492 | 0.000877 | 0 | 0 | 0 |
| rf4 | 0.001419 | 0.000677 | 0.004836 | 0.00786 | 0.004419 | 0.000361 | 0.001757 | 0.000625 | 0.000788 | 0.000304 |
| rf5 | 0.002669 | 0 | 0.000315 | 0.007807 | 0 | 0.002615 | 0 | 0.003275 | 0.001195 | 0.002216 |
| rf6 | 0 | 0 | 0.003732 | 0.003619 | 0.005642 | 0.011649 | 0.001156 | 3.54E−18 | 0.002721 | 0.001778 |
| rf7 | 0.001414 | 0.005904 | 0 | 0.00178 | 0.002299 | 0 | 0.001072 | 0.001608 | 0 | 0.00377 |
| rf8 | 0 | 0.002075 | 0 | 0.002899 | 1.49E−18 | 0.000592 | 0.000773 | 0.002271 | 0.001039 | 0.002886 |
| rf9 | 0.001851 | 0 | 0.002647 | 0.008929 | 6.06E−19 | 0.004954 | 1.56E−18 | 0 | 0 | 0.002541 |
| rf10 | 0.005401 | 0.001061 | 0.000776 | 0.002372 | 0.001064 | 5.35E−19 | 0.008113 | 0.001074 | 0.002219 | 0.00333 |
| rf11 | 0 | 0.000794 | 0.006158 | 0.005197 | 0.003866 | 0 | 0 | 0.003205 | 0.006227 | 0.001826 |
| rf12 | 0.001603 | 3.96E−18 | 0.000804 | 0 | 0.003321 | 0.000285 | 0.000769 | 0 | 1.30E−18 | 0.000768 |
| rf13 | 0 | 9.43E−18 | 0.000591 | 0.00693 | 0 | 0 | 0 | 0 | 0.00198 | 0.001646 |
| rf14 | 0.001134 | 0.000737 | 0.004315 | 0.002797 | 0.005707 | 0 | 0.003375 | 0 | 0.002982 | 9.58E−05 |
| rf15 | 0 | 0.005724 | 0.00431 | 0.009576 | 0.003918 | 3.27E−18 | 0 | 0.001857 | 0.000288 | 0.005228 |
| rf16 | 0 | 0.004923 | 0 | 0.003785 | 0 | 0.00165 | 0 | 7.55E−19 | 0.002045 | 0.001984 |
| rf17 | 0.00036 | 0 | 0.003583 | 0.000339 | 0 | 0.001492 | 0.000877 | 0 | 0 | 0 |
| rf18 | 0.001419 | 0.000677 | 0.004836 | 0.00786 | 0.004419 | 0.000361 | 0.001757 | 0.000625 | 0.000788 | 0.000304 |
| rf19 | 0.002669 | 0 | 0.000315 | 0.007807 | 0 | 0.002615 | 0 | 0.003275 | 0.001195 | 0.002216 |
| rf20 | 0 | 0 | 0.003732 | 0.003619 | 0.005642 | 0.011649 | 0.001156 | 3.54E−18 | 0.002721 | 0.001778 |
| rf21 | 0.001414 | 0.005904 | 0 | 0.00178 | 0.002299 | 0 | 0.001072 | 0.001608 | 0 | 0.00377 |
| rf22 | 0 | 0.002075 | 0 | 0.002899 | 1.49E−18 | 0.000592 | 0.000773 | 0.002271 | 0.001039 | 0.002886 |
| rf23 | 0.001851 | 0 | 0.002647 | 0.008929 | 6.06E−19 | 0.004954 | 1.56E−18 | 0 | 0 | 0.002541 |
| rf24 | 0.005401 | 0.001061 | 0.000776 | 0.002372 | 0.001064 | 5.35E−19 | 0.008113 | 0.001074 | 0.002219 | 0.00333 |
| rf25 | 0 | 0.000794 | 0.006158 | 0.005197 | 0.003866 | 0 | 0 | 0.003205 | 0.006227 | 0.001826 |
| rf26 | 0.001603 | 3.96E−18 | 0.000804 | 0 | 0.003321 | 0.000285 | 0.000769 | 0 | 1.30E−18 | 0.000768 |
| rf27 | 0 | 9.43E−18 | 0.000591 | 0.00693 | 0 | 0 | 0 | 0 | 0.00198 | 0.001646 |
| extraTrees0 | 0.003052 | 0 | 0.001864 | 0.001653 | 0.003924 | 0.001276 | 0.001386 | 0.003558 | 0.000835 | 0.001665 |
| extraTrees1 | 0.002805 | 0.001696 | 0.003811 | 0.003933 | 0.001965 | 0.001126 | 0.001182 | 0.003106 | 0.000683 | 0.000546 |
| extraTrees2 | 0.000338 | 0.001818 | 0.002253 | 0.002802 | 0.002885 | 3.38E−17 | 0.001971 | 0.003752 | 0.001471 | 0.000519 |
| extraTrees3 | 0.001119 | 4.90E−05 | 0.003326 | 0.003216 | 0.000525 | 0.000143 | 0.000731 | 0.004439 | 0.001665 | 0.002356 |
| extraTrees4 | 0.000774 | 0.00283 | 0.002007 | 0.003459 | 0.002555 | 0.00061 | 2.49E−05 | 0.003973 | 0 | 0.002304 |
| extraTrees5 | 0.003006 | 0.002212 | 0.000387 | 0.005348 | 0.001159 | 0.001524 | 0.000807 | 0.003455 | 0.000748 | 0.001061 |
| extraTrees6 | 0.000898 | 0.001362 | 0.000301 | 0.003622 | 0.000287 | 0.001527 | 0.001101 | 0 | 0.001588 | 0.001868 |
| extraTrees7 | 0.00101 | 0.001245 | 7.62E−18 | 0.002561 | 0.000767 | 0.002792 | 0.00028 | 0.000252 | 0.000777 | 0.002577 |
| extraTrees8 | 0.002178 | 0.002276 | 0.001744 | 0.00111 | 0.002342 | 0.000612 | 0.001471 | 0.000388 | 0.002681 | 0.000567 |
| extraTrees9 | 0.001804 | 0.000739 | 0.001822 | 0.002097 | 0.002541 | 0.001003 | 0.000256 | 0.002703 | 0.000238 | 0.001984 |
| extraTrees10 | 0.001144 | 0.001374 | 0.004995 | 0.001849 | 0.001351 | 0.002959 | 0 | 0.002133 | 0.00268 | 0.00514 |
| extraTrees11 | 0.0007 | 0.000843 | 0.001516 | 0.001978 | 0.001033 | 0.000387 | 0.001024 | 0.001537 | 0.000417 | 0.001924 |
| extraTrees12 | 0.000911 | 0.001667 | 0.000477 | 0.001263 | 0.003147 | 0.00068 | 0.002281 | 0.004306 | 4.76E−17 | 0.003132 |
| extraTrees13 | 0.000866 | 2.39E−18 | 0.005624 | 0.004024 | 0.001602 | 0.001582 | 0.00318 | 0.001127 | 0.000592 | 0.00253 |
| extraTrees14 | 0.003052 | 0 | 0.001864 | 0.001653 | 0.003924 | 0.001276 | 0.001386 | 0.003558 | 0.000835 | 0.001665 |
| extraTrees15 | 0.002805 | 0.001696 | 0.003811 | 0.003933 | 0.001965 | 0.001126 | 0.001182 | 0.003106 | 0.000683 | 0.000546 |
| extraTrees16 | 0.000338 | 0.001818 | 0.002253 | 0.002802 | 0.002885 | 3.38E−17 | 0.001971 | 0.003752 | 0.001471 | 0.000519 |
| extraTrees17 | 0.001119 | 4.90E−05 | 0.003326 | 0.003216 | 0.000525 | 0.000143 | 0.000731 | 0.004439 | 0.001665 | 0.002356 |
| extraTrees18 | 0.000774 | 0.00283 | 0.002007 | 0.003459 | 0.002555 | 0.00061 | 2.49E−05 | 0.003973 | 0 | 0.002304 |
| extraTrees19 | 0.003006 | 0.002212 | 0.000387 | 0.005348 | 0.001159 | 0.001524 | 0.000807 | 0.003455 | 0.000748 | 0.001061 |
| extraTrees20 | 0.000898 | 0.001362 | 0.000301 | 0.003622 | 0.000287 | 0.001527 | 0.001101 | 0 | 0.001588 | 0.001868 |
| extraTrees21 | 0.00101 | 0.001245 | 7.62E−18 | 0.002561 | 0.000767 | 0.002792 | 0.00028 | 0.000252 | 0.000777 | 0.002577 |
| extraTrees22 | 0.002178 | 0.002276 | 0.001744 | 0.00111 | 0.002342 | 0.000612 | 0.001471 | 0.000388 | 0.002681 | 0.000567 |
| extraTrees23 | 0.001804 | 0.000739 | 0.001822 | 0.002097 | 0.002541 | 0.001003 | 0.000256 | 0.002703 | 0.000238 | 0.001984 |
| extraTrees24 | 0.001144 | 0.001374 | 0.004995 | 0.001849 | 0.001351 | 0.002959 | 0 | 0.002133 | 0.00268 | 0.00514 |
| extraTrees25 | 0.0007 | 0.000843 | 0.001516 | 0.001978 | 0.001033 | 0.000387 | 0.001024 | 0.001537 | 0.000417 | 0.001924 |
| extraTrees26 | 0.000911 | 0.001667 | 0.000477 | 0.001263 | 0.003147 | 0.00068 | 0.002281 | 0.004306 | 4.76E−17 | 0.003132 |
| extraTrees27 | 0.000866 | 2.39E−18 | 0.005624 | 0.004024 | 0.001602 | 0.001582 | 0.00318 | 0.001127 | 0.000592 | 0.00253 |
| gbc0 | 0 | 0.016699 | 0.000893 | 0.002259 | 0 | 0 | 0.019999 | 0.001688 | 0.000161 | 0 |
| gbc1 | 3.88E−05 | 0 | 0 | 2.79E−05 | 0 | 0 | 0 | 0.00306 | 0 | 0 |
| gbc2 | 0.003166 | 0 | 0.00562 | 0.000234 | 0 | 0 | 2.70E−06 | 0.056703 | 1.59E−08 | 0 |
| gbc3 | 0.000459 | 0.002621 | 0 | 0.018076 | 3.57E−05 | 2.78E−05 | 0.008486 | 0.00439 | 0 | 0 |
| gbc4 | 3.41E−05 | 0.007846 | 0 | 0.003512 | 0.005085 | 0.000658 | 0.003411 | 0.036895 | 2.54E−08 | 0 |
| gbc5 | 0 | 0 | 5.49E−06 | 0.005097 | 0 | 0 | 0 | 0.015734 | 0.001756 | 0 |
| gbc6 | 0.001079 | 4.98E−05 | 0.008939 | 0.001275 | 0.003229 | 0 | 0 | 0.061782 | 0.002321 | 0 |
| gbc7 | 0.000346 | 0 | 0.002382 | 0.020065 | 4.00E−05 | 0 | 0 | 0.000514 | 0 | 0 |
| gbc8 | 0 | 0.015089 | 0.000177 | 0.000858 | 0.000148 | 0 | 0 | 0 | 0 | 0.00684 |
| gbc9 | 0 | 0 | 0 | 0.001146 | 0 | 2.37E−06 | 0 | 0.058922 | 0 | 0 |
| gbc10 | 0 | 0.007883 | 0 | 0.005697 | 0.006799 | 0.000388 | 0.000504 | 0.000546 | 0.009013 | 0.001615 |
| gbc11 | 0 | 0 | 0.001927 | 0.000714 | 0.007925 | 0 | 0 | 0.03306 | 0 | 0.000105 |
| gbc12 | 0 | 0.000275 | 0 | 0.000651 | 0.010462 | 2.57E−05 | 0 | 0.000109 | 0 | 0.000353 |
| gbc13 | 0.001064 | 0.014443 | 0.001361 | 0.0102 | 0 | 0.00032 | 0.03029 | 0.01313 | 7.54E−05 | 0.006003 |
| gbc14 | 0 | 0.016699 | 0.000893 | 0.002259 | 0 | 0 | 0.019999 | 0.001688 | 0.000161 | 0 |
| gbc15 | 3.88E−05 | 0 | 0 | 2.79E−05 | 0 | 0 | 0 | 0.00306 | 0 | 0 |
| gbc16 | 0.003166 | 0 | 0.00562 | 0.000234 | 0 | 0 | 2.70E−06 | 0.056703 | 1.59E−08 | 0 |
| gbc17 | 0.000459 | 0.002621 | 0 | 0.018076 | 3.57E−05 | 2.78E−05 | 0.008486 | 0.00439 | 0 | 0 |
| gbc18 | 3.41E−05 | 0.007846 | 0 | 0.003512 | 0.005085 | 0.000658 | 0.003411 | 0.036895 | 2.54E−08 | 0 |
| gbc19 | 0 | 0 | 5.49E−06 | 0.005097 | 0 | 0 | 0 | 0.015734 | 0.001756 | 0 |
| gbc20 | 0.001079 | 4.98E−05 | 0.008939 | 0.001275 | 0.003229 | 0 | 0 | 0.061782 | 0.002321 | 0 |
| gbc21 | 0.000346 | 0 | 0.002382 | 0.020065 | 4.00E−05 | 0 | 0 | 0.000514 | 0 | 0 |
| gbc22 | 0 | 0.015089 | 0.000177 | 0.000858 | 0.000148 | 0 | 0 | 0 | 0 | 0.00684 |
| gbc23 | 0 | 0 | 0 | 0.001146 | 0 | 2.37E−06 | 0 | 0.058922 | 0 | 0 |
| gbc24 | 0 | 0.007883 | 0 | 0.005697 | 0.006799 | 0.000388 | 0.000504 | 0.000546 | 0.009013 | 0.001615 |
| gbc25 | 0 | 0 | 0.001927 | 0.000714 | 0.007925 | 0 | 0 | 0.03306 | 0 | 0.000105 |
| gbc26 | 0 | 0.000275 | 0 | 0.000651 | 0.010462 | 2.57E−05 | 0 | 0.000109 | 0 | 0.000353 |
| gbc27 | 0.001064 | 0.014443 | 0.001361 | 0.0102 | 0 | 0.00032 | 0.03029 | 0.01313 | 7.54E−05 | 0.006003 |
| ada0 | 0 | 0 | 0 | 0.02 | 0 | 0 | 0 | 0 | 0 | 0 |
| ada1 | 0 | 0 | 0 | 0.02 | 0 | 0 | 0 | 0 | 0 | 0 |
| ada2 | 0 | 0 | 0 | 0.02 | 0 | 0 | 0 | 0 | 0 | 0 |
| ada3 | 0 | 0 | 0 | 0.02 | 0 | 0 | 0 | 0 | 0 | 0 |
| ada4 | 0 | 0 | 0 | 0.02 | 0 | 0 | 0 | 0 | 0 | 0 |
| ada5 | 0 | 0 | 0 | 0.02 | 0 | 0 | 0 | 0 | 0 | 0 |
| ada6 | 0 | 0 | 0 | 0.02 | 0 | 0 | 0 | 0 | 0 | 0 |
| ada7 | 0 | 0 | 0 | 0.02 | 0 | 0 | 0 | 0 | 0 | 0 |
| ada8 | 0 | 0 | 0 | 0.02 | 0 | 0 | 0 | 0 | 0 | 0 |
| ada9 | 0 | 0 | 0 | 0.02 | 0 | 0 | 0 | 0 | 0 | 0 |
| ada10 | 0 | 0 | 0 | 0.02 | 0 | 0 | 0 | 0 | 0 | 0 |
| ada11 | 0 | 0 | 0 | 0.02 | 0 | 0 | 0 | 0 | 0 | 0 |
| ada12 | 0 | 0 | 0 | 0.02 | 0 | 0 | 0 | 0 | 0 | 0 |
| ada13 | 0 | 0 | 0 | 0.02 | 0 | 0 | 0 | 0 | 0 | 0 |
| ada14 | 0 | 0 | 0 | 0.02 | 0 | 0 | 0 | 0 | 0 | 0 |
| ada15 | 0 | 0 | 0 | 0.02 | 0 | 0 | 0 | 0 | 0 | 0 |
| ada16 | 0 | 0 | 0 | 0.02 | 0 | 0 | 0 | 0 | 0 | 0 |
| ada17 | 0 | 0 | 0 | 0.02 | 0 | 0 | 0 | 0 | 0 | 0 |
| ada18 | 0 | 0 | 0 | 0.02 | 0 | 0 | 0 | 0 | 0 | 0 |
| ada19 | 0 | 0 | 0 | 0.02 | 0 | 0 | 0 | 0 | 0 | 0 |
| ada20 | 0 | 0 | 0 | 0.02 | 0 | 0 | 0 | 0 | 0 | 0 |
| ada21 | 0 | 0 | 0 | 0.02 | 0 | 0 | 0 | 0 | 0 | 0 |
| ada22 | 0 | 0 | 0 | 0.02 | 0 | 0 | 0 | 0 | 0 | 0 |
| ada23 | 0 | 0 | 0 | 0.02 | 0 | 0 | 0 | 0 | 0 | 0 |
| ada24 | 0 | 0 | 0 | 0.02 | 0 | 0 | 0 | 0 | 0 | 0 |
| ada25 | 0 | 0 | 0 | 0.02 | 0 | 0 | 0 | 0 | 0 | 0 |
| ada26 | 0 | 0 | 0 | 0.02 | 0 | 0 | 0 | 0 | 0 | 0 |
| ada27 | 0 | 0 | 0 | 0.02 | 0 | 0 | 0 | 0 | 0 | 0 |
| Gene | PLCH1 | PLIN3 | POMGNT2 | POT1 | PPHLN1 | PPIL3 | PPIL4 | PPP1R2 | PRKCA | PRMT3 |
| rf0 | 0.001929 | 0.001568 | 0.000449 | 0.009698 | 0.001485 | 0.003742 | 0.008627 | 0 | 0 | 0.004092 |
| rf1 | 0.002179 | 0.000722 | 0.000703 | 0.012279 | 0 | 0 | 0.0056 | 0 | 0.000342 | 0 |
| rf2 | 2.67E−19 | 0.000293 | 0 | 0.011352 | 0 | 0.004193 | 0 | 0.003649 | 0 | 0.000365 |
| rf3 | 0.001008 | 0 | 2.45E−19 | 0.009455 | 0.000651 | 0 | 0.00419 | 0 | 0.001194 | 0.002489 |
| rf4 | 0.001266 | 0 | 0.002027 | 0.009666 | 0 | 0.001171 | 0.009651 | 0 | 0.00272 | 0 |
| rf5 | 2.66E−18 | 0 | 0 | 0.006122 | 0.00085 | 0.006859 | 0 | 0.002578 | 0.00216 | 0 |
| rf6 | 0.001065 | 0.00041 | 0 | 0.00178 | 0.002502 | 0.001159 | 0.003109 | 0.001978 | 0.006026 | 0.003477 |
| rf7 | 0.001713 | 0.001246 | 5.45E−19 | 0.002733 | 0.002679 | 0.002129 | 0.001278 | 0.001387 | 4.06E−18 | 0 |
| rf8 | 0.003162 | 0.002311 | 0.001027 | 0.001802 | 0 | 0.002929 | 0.003541 | 0.001434 | 0.002226 | 0 |
| rf9 | 0.000359 | 0 | 0.000963 | 0.003274 | 0.001378 | 0.000158 | 0.00204 | 0 | 0 | 0.000342 |
| rf10 | 0.002254 | 0 | 0 | 0.012337 | 0.005434 | 0 | 0.000395 | 0.001187 | 0.000324 | 0.001147 |
| rf11 | 0 | 0.005176 | 0.001078 | 0.010483 | 0.002149 | 0.001738 | 0 | 0 | 0.001285 | 0.001648 |
| rf12 | 0.001299 | 0 | 0.00037 | 0.005924 | 0 | 0 | 0 | 0 | 0 | 0 |
| rf13 | 0.002352 | 0 | 0 | 0.001363 | 0.001417 | 0 | 0.002237 | 0.001261 | 0 | 0.002631 |
| rf14 | 0.001929 | 0.001568 | 0.000449 | 0.009698 | 0.001485 | 0.003742 | 0.008627 | 0 | 0 | 0.004092 |
| rf15 | 0.002179 | 0.000722 | 0.000703 | 0.012279 | 0 | 0 | 0.0056 | 0 | 0.000342 | 0 |
| rf16 | 2.67E−19 | 0.000293 | 0 | 0.011352 | 0 | 0.004193 | 0 | 0.003649 | 0 | 0.000365 |
| rf17 | 0.001008 | 0 | 2.45E−19 | 0.009455 | 0.000651 | 0 | 0.00419 | 0 | 0.001194 | 0.002489 |
| rf18 | 0.001266 | 0 | 0.002027 | 0.009666 | 0 | 0.001171 | 0.009651 | 0 | 0.00272 | 0 |
| rf19 | 2.66E−18 | 0 | 0 | 0.006122 | 0.00085 | 0.006859 | 0 | 0.002578 | 0.00216 | 0 |
| rf20 | 0.001065 | 0.00041 | 0 | 0.00178 | 0.002502 | 0.001159 | 0.003109 | 0.001978 | 0.006026 | 0.003477 |
| rf21 | 0.001713 | 0.001246 | 5.45E−19 | 0.002733 | 0.002679 | 0.002129 | 0.001278 | 0.001387 | 4.06E−18 | 0 |
| rf22 | 0.003162 | 0.002311 | 0.001027 | 0.001802 | 0 | 0.002929 | 0.003541 | 0.001434 | 0.002226 | 0 |
| rf23 | 0.000359 | 0 | 0.000963 | 0.003274 | 0.001378 | 0.000158 | 0.00204 | 0 | 0 | 0.000342 |
| rf24 | 0.002254 | 0 | 0 | 0.012337 | 0.005434 | 0 | 0.000395 | 0.001187 | 0.000324 | 0.001147 |
| rf25 | 0 | 0.005176 | 0.001078 | 0.010483 | 0.002149 | 0.001738 | 0 | 0 | 0.001285 | 0.001648 |
| rf26 | 0.001299 | 0 | 0.00037 | 0.005924 | 0 | 0 | 0 | 0 | 0 | 0 |
| rf27 | 0.002352 | 0 | 0 | 0.001363 | 0.001417 | 0 | 0.002237 | 0.001261 | 0 | 0.002631 |
| extraTrees0 | 0.001164 | 0.000525 | 0.001428 | 0.001932 | 0.003547 | 0.004987 | 0.001965 | 0.000542 | 0.004781 | 0.001699 |
| extraTrees1 | 0.002808 | 0.000764 | 0.003008 | 0.006999 | 0.001557 | 0.001317 | 0.000612 | 0.001291 | 0.003488 | 0.001554 |
| extraTrees2 | 0.000852 | 0.000423 | 0.000847 | 0.003145 | 0.001214 | 0.001891 | 0.002573 | 0.000938 | 0.003018 | 0.001319 |
| extraTrees3 | 0.001805 | 0 | 0.00438 | 0.002005 | 3.59E−18 | 0.003274 | 0.004977 | 1.26E−18 | 0.004299 | 0.001031 |
| extraTrees4 | 0.001333 | 0 | 0.001035 | 0.012746 | 0.001914 | 0.005407 | 0.00359 | 0 | 0.003227 | 0.00102 |
| extraTrees5 | 0.002626 | 0.000405 | 0.001938 | 0.006636 | 0.000935 | 0.002723 | 0.001461 | 2.14E−18 | 0.004941 | 0.00159 |
| extraTrees6 | 0.00193 | 0.00095 | 0.000567 | 0.004601 | 0.000388 | 0.004323 | 0 | 0.000607 | 0.003278 | 0.000627 |
| extraTrees7 | 0.00028 | 0.002203 | 0.002146 | 0.002545 | 1.47E−18 | 0.003444 | 0.000935 | 0.000268 | 0.005147 | 0.000238 |
| extraTrees8 | 0.000928 | 0.00062 | 0.000608 | 0.003085 | 0.001824 | 0.002336 | 0.002227 | 0.000515 | 0.003178 | 0.003478 |
| extraTrees9 | 0.00235 | 0.001625 | 0.003933 | 0.003756 | 0.001213 | 0.001524 | 0.003744 | 0.001441 | 0.002782 | 0.000811 |
| extraTrees10 | 0.00187 | 0.001735 | 0.001345 | 0.004672 | 5.26E−19 | 0.000567 | 0.004599 | 0.000685 | 0.003792 | 0.000973 |
| extraTrees11 | 0.002202 | 0.000576 | 0.001363 | 0.008522 | 0.001253 | 0.00165 | 0.002113 | 0.000258 | 0.00122 | 0.005601 |
| extraTrees12 | 0.002759 | 0.001074 | 0.000806 | 0.005642 | 0.000492 | 0.00333 | 0.000285 | 0.000208 | 0.003084 | 0.003252 |
| extraTrees13 | 0.000447 | 0.001355 | 0.002099 | 0.001854 | 0.00203 | 0.002222 | 0.003013 | 0.000674 | 0.001157 | 0.000708 |
| extraTrees14 | 0.001164 | 0.000525 | 0.001428 | 0.001932 | 0.003547 | 0.004987 | 0.001965 | 0.000542 | 0.004781 | 0.001699 |
| extraTrees15 | 0.002808 | 0.000764 | 0.003008 | 0.006999 | 0.001557 | 0.001317 | 0.000612 | 0.001291 | 0.003488 | 0.001554 |
| extraTrees16 | 0.000852 | 0.000423 | 0.000847 | 0.003145 | 0.001214 | 0.001891 | 0.002573 | 0.000938 | 0.003018 | 0.001319 |
| extraTrees17 | 0.001805 | 0 | 0.00438 | 0.002005 | 3.59E−18 | 0.003274 | 0.004977 | 1.26E−18 | 0.004299 | 0.001031 |
| extraTrees18 | 0.001333 | 0 | 0.001035 | 0.012746 | 0.001914 | 0.005407 | 0.00359 | 0 | 0.003227 | 0.00102 |
| extraTrees19 | 0.002626 | 0.000405 | 0.001938 | 0.006636 | 0.000935 | 0.002723 | 0.001461 | 2.14E−18 | 0.004941 | 0.00159 |
| extraTrees20 | 0.00193 | 0.00095 | 0.000567 | 0.004601 | 0.000388 | 0.004323 | 0 | 0.000607 | 0.003278 | 0.000627 |
| extraTrees21 | 0.00028 | 0.002203 | 0.002146 | 0.002545 | 1.47E−18 | 0.003444 | 0.000935 | 0.000268 | 0.005147 | 0.000238 |
| extraTrees22 | 0.000928 | 0.00062 | 0.000608 | 0.003085 | 0.001824 | 0.002336 | 0.002227 | 0.000515 | 0.003178 | 0.003478 |
| extraTrees23 | 0.00235 | 0.001625 | 0.003933 | 0.003756 | 0.001213 | 0.001524 | 0.003744 | 0.001441 | 0.002782 | 0.000811 |
| extraTrees24 | 0.00187 | 0.001735 | 0.001345 | 0.004672 | 5.26E−19 | 0.000567 | 0.004599 | 0.000685 | 0.003792 | 0.000973 |
| extraTrees25 | 0.002202 | 0.000576 | 0.001363 | 0.008522 | 0.001253 | 0.00165 | 0.002113 | 0.000258 | 0.00122 | 0.005601 |
| extraTrees26 | 0.002759 | 0.001074 | 0.000806 | 0.005642 | 0.000492 | 0.00333 | 0.000285 | 0.000208 | 0.003084 | 0.003252 |
| extraTrees27 | 0.000447 | 0.001355 | 0.002099 | 0.001854 | 0.00203 | 0.002222 | 0.003013 | 0.000674 | 0.001157 | 0.000708 |
| gbc0 | 0 | 5.80E−05 | 0.002158 | 0.000136 | 0 | 0 | 0 | 0.014681 | 0 | 0 |
| gbc1 | 2.95E−05 | 0 | 0.000938 | 0 | 0 | 0.001835 | 0 | 0 | 0.000231 | 0.002063 |
| gbc2 | 0 | 0 | 0.010936 | 0 | 0.000159 | 2.00E−05 | 0 | 0 | 0 | 0.022493 |
| gbc3 | 0 | 8.67E−06 | 0 | 0 | 0 | 0 | 0 | 0 | 6.03E−05 | 0 |
| gbc4 | 0 | 0 | 4.81E−05 | 0.007909 | 1.41E−05 | 0.000111 | 2.58E−05 | 0.013771 | 0.001776 | 6.38E−05 |
| gbc5 | 2.91E−05 | 8.99E−06 | 0 | 0 | 1.79E−06 | 0.000961 | 0.005545 | 0.001208 | 3.42E−05 | 0 |
| gbc6 | 0 | 0 | 0 | 0 | 0 | 0 | 4.65E−06 | 0 | 0 | 0 |
| gbc7 | 0 | 0.000308 | 0 | 0.028802 | 0.01284 | 0 | 2.51E−06 | 6.24E−06 | 0.002104 | 0.001275 |
| gbc8 | 0.000101 | 0.015393 | 0.021056 | 0 | 0 | 0.004656 | 0 | 5.05E−07 | 4.53E−05 | 0 |
| gbc9 | 0 | 0.000244 | 2.27E−05 | 0.010647 | 0 | 0.004983 | 0 | 0.001169 | 0.025139 | 0 |
| gbc10 | 4.07E−05 | 0 | 0 | 0 | 0.000645 | 0 | 0 | 0 | 0 | 0.002099 |
| gbc11 | 0.001109 | 1.56E−05 | 0.005483 | 3.55E−05 | 2.53E−06 | 0 | 3.53E−05 | 0.008369 | 1.90E−05 | 2.01E−05 |
| gbc12 | 7.26E−05 | 0 | 0.000198 | 0 | 0.000314 | 0 | 0 | 0 | 0.021261 | 0 |
| gbc13 | 0 | 2.27E−05 | 1.84E−06 | 0 | 3.30E−05 | 3.61E−09 | 0 | 0.000783 | 0.00074 | 0 |
| gbc14 | 0 | 5.80E−05 | 0.002158 | 0.000136 | 0 | 0 | 0 | 0.014681 | 0 | 0 |
| gbc15 | 2.95E−05 | 0 | 0.000938 | 0 | 0 | 0.001835 | 0 | 0 | 0.000231 | 0.002063 |
| gbc16 | 0 | 0 | 0.010936 | 0 | 0.000159 | 2.00E−05 | 0 | 0 | 0 | 0.022493 |
| gbc17 | 0 | 8.67E−06 | 0 | 0 | 0 | 0 | 0 | 0 | 6.03E−05 | 0 |
| gbc18 | 0 | 0 | 4.81E−05 | 0.007909 | 1.41E−05 | 0.000111 | 2.58E−05 | 0.013771 | 0.001776 | 6.38E−05 |
| gbc19 | 2.91E−05 | 8.99E−06 | 0 | 0 | 1.79E−06 | 0.000961 | 0.005545 | 0.001208 | 3.42E−05 | 0 |
| gbc20 | 0 | 0 | 0 | 0 | 0 | 0 | 4.65E−06 | 0 | 0 | 0 |
| gbc21 | 0 | 0.000308 | 0 | 0.028802 | 0.01284 | 0 | 2.51E−06 | 6.24E−06 | 0.002104 | 0.001275 |
| gbc22 | 0.000101 | 0.015393 | 0.021056 | 0 | 0 | 0.004656 | 0 | 5.05E−07 | 4.53E−05 | 0 |
| gbc23 | 0 | 0.000244 | 2.27E−05 | 0.010647 | 0 | 0.004983 | 0 | 0.001169 | 0.025139 | 0 |
| gbc24 | 4.07E−05 | 0 | 0 | 0 | 0.000645 | 0 | 0 | 0 | 0 | 0.002099 |
| gbc25 | 0.001109 | 1.56E−05 | 0.005483 | 3.55E−05 | 2.53E−06 | 0 | 3.53E−05 | 0.008369 | 1.90E−05 | 2.01E−05 |
| gbc26 | 7.26E−05 | 0 | 0.000198 | 0 | 0.000314 | 0 | 0 | 0 | 0.021261 | 0 |
| gbc27 | 0 | 2.27E−05 | 1.84E−06 | 0 | 3.30E−05 | 3.61E−09 | 0 | 0.000783 | 0.00074 | 0 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |
| Gene | PSMD10 | PTBP3 | PTMAP9 | RAB13 | RAB1A | RAB2B | RB1 | RB1CC1 | RBM12B | RBM7 |
| rf0 | 0.001378 | 0 | 0.0016 | 0.000717 | 0 | 0.00241 | 0.004954 | 0.001633 | 0.006282 | 0.003392 |
| rf1 | 0.003787 | 0.006067 | 0.00252 | 0 | 0.010941 | 0.002145 | 0.001286 | 0 | 0.005197 | 0.006622 |
| rf2 | 0.003565 | 0.005661 | 0.00198 | 0.00182 | 0.005323 | 0.002037 | 0.00091 | 0 | 0.002544 | 0 |
| rf3 | 0.001164 | 8.12E−19 | 0.000319 | 0 | 0 | 0.004454 | 0 | 0.001525 | 0.001647 | 0 |
| rf4 | 0.00035 | 0.001406 | 0.001767 | 0.000224 | 0.00957 | 6.00E−18 | 0.001028 | 0 | 0.001101 | 0 |
| rf5 | 0.005174 | 0 | 0.000366 | 0.005283 | 0.009493 | 0.000663 | 0.003917 | 0 | 0.003872 | 0 |
| rf6 | 0.002362 | 0.001216 | 0.003111 | 0.006684 | 0.001011 | 0.002087 | 0.001393 | 0.001868 | 0.004901 | 0 |
| rf7 | 2.45E−19 | 0 | 0.00036 | 0.002611 | 0.005483 | 0.003795 | 0 | 0.001328 | 0 | 0 |
| rf8 | 0.009643 | 0 | 0.001353 | 0.007571 | 0.003421 | 0.004622 | 0.002236 | 0 | 0 | 0 |
| rf9 | 0.000465 | 0.003277 | 0 | 0.003606 | 0.007314 | 0.001782 | 0.002217 | 0.00203 | 0.002404 | 0.001901 |
| rf10 | 0.002041 | 0.004474 | 0.001056 | 0.001131 | 0 | 0 | 0.005599 | 0.001042 | 0.006101 | 0 |
| rf11 | 0.000999 | 0 | 0.002676 | 0 | 0 | 0.006624 | 0 | 0.001844 | 0.006182 | 0 |
| rf12 | 0 | 0 | 0 | 0.000545 | 0.005141 | 0.000638 | 0.000399 | 0.000672 | 0 | 0.001825 |
| rf13 | 0.001294 | 0.016422 | 0 | 0 | 0.005386 | 0 | 2.56E−19 | 0 | 0.005522 | 0 |
| rf14 | 0.001378 | 0 | 0.0016 | 0.000717 | 0 | 0.00241 | 0.004954 | 0.001633 | 0.006282 | 0.003392 |
| rf15 | 0.003787 | 0.006067 | 0.00252 | 0 | 0.010941 | 0.002145 | 0.001286 | 0 | 0.005197 | 0.006622 |
| rf16 | 0.003565 | 0.005661 | 0.00198 | 0.00182 | 0.005323 | 0.002037 | 0.00091 | 0 | 0.002544 | 0 |
| rf17 | 0.001164 | 8.12E−19 | 0.000319 | 0 | 0 | 0.004454 | 0 | 0.001525 | 0.001647 | 0 |
| rf18 | 0.00035 | 0.001406 | 0.001767 | 0.000224 | 0.00957 | 6.00E−18 | 0.001028 | 0 | 0.001101 | 0 |
| rf19 | 0.005174 | 0 | 0.000366 | 0.005283 | 0.009493 | 0.000663 | 0.003917 | 0 | 0.003872 | 0 |
| rf20 | 0.002362 | 0.001216 | 0.003111 | 0.006684 | 0.001011 | 0.002087 | 0.001393 | 0.001868 | 0.004901 | 0 |
| rf21 | 2.45E−19 | 0 | 0.00036 | 0.002611 | 0.005483 | 0.003795 | 0 | 0.001328 | 0 | 0 |
| rf22 | 0.009643 | 0 | 0.001353 | 0.007571 | 0.003421 | 0.004622 | 0.002236 | 0 | 0 | 0 |
| rf23 | 0.000465 | 0.003277 | 0 | 0.003606 | 0.007314 | 0.001782 | 0.002217 | 0.00203 | 0.002404 | 0.001901 |
| rf24 | 0.002041 | 0.004474 | 0.001056 | 0.001131 | 0 | 0 | 0.005599 | 0.001042 | 0.006101 | 0 |
| rf25 | 0.000999 | 0 | 0.002676 | 0 | 0 | 0.006624 | 0 | 0.001844 | 0.006182 | 0 |
| rf26 | 0 | 0 | 0 | 0.000545 | 0.005141 | 0.000638 | 0.000399 | 0.000672 | 0 | 0.001825 |
| rf27 | 0.001294 | 0.016422 | 0 | 0 | 0.005386 | 0 | 2.56E−19 | 0 | 0.005522 | 0 |
| extraTrees0 | 0.001532 | 0.003806 | 0.000933 | 0.000844 | 0.002657 | 0.00423 | 0.001866 | 0.000494 | 0.006851 | 0.001371 |
| extraTrees1 | 0.000626 | 0.000562 | 0.000864 | 0 | 0.001274 | 0.001371 | 0.002177 | 0.000287 | 0.002243 | 0.001 |
| extraTrees2 | 0 | 0.00025 | 0.000238 | 0.000238 | 0.00172 | 0.002394 | 0.001366 | 0.001226 | 0.003912 | 0.000369 |
| extraTrees3 | 0.001124 | 0.000472 | 0.001101 | 0.000567 | 0.001567 | 0.003095 | 0.000518 | 0.001739 | 0.003681 | 0.000676 |
| extraTrees4 | 0.001158 | 0.004055 | 0.000959 | 0.005602 | 0.00312 | 0.001161 | 0.000238 | 0.001466 | 0.00233 | 1.26E−18 |
| extraTrees5 | 0.000336 | 0.002558 | 0.003091 | 0.001205 | 0.002679 | 0.001692 | 0.002442 | 0 | 0.001661 | 0.001629 |
| extraTrees6 | 0.001865 | 0.002673 | 0.000415 | 0.006357 | 0.003106 | 0.001863 | 0.002404 | 0 | 0.003564 | 0.001245 |
| extraTrees7 | 0.001136 | 1.23E−18 | 0.001514 | 0.000503 | 0.000984 | 0.001458 | 0.001442 | 0.002068 | 0.001647 | 0.001418 |
| extraTrees8 | 0.002177 | 0.00131 | 0.000492 | 0.004528 | 0.0087 | 0.001051 | 0.00109 | 0.000643 | 0.004415 | 5.26E−05 |
| extraTrees9 | 0.000676 | 0.000781 | 0.001563 | 0.000653 | 0.00479 | 0 | 0.001134 | 0.000909 | 0.001879 | 0 |
| extraTrees10 | 0.001576 | 0.003887 | 0.002771 | 0.001599 | 0.001092 | 0.004554 | 0.00159 | 0.001302 | 0.002762 | 0.000907 |
| extraTrees11 | 0.001405 | 0.001002 | 0.000238 | 0.003915 | 0.00315 | 0.002059 | 5.10E−17 | 0.001358 | 0.001427 | 0.001323 |
| extraTrees12 | 0.002291 | 0.002467 | 0.000388 | 0.001037 | 0.004066 | 0.000948 | 0.000625 | 0.000657 | 0.000777 | 0.001594 |
| extraTrees13 | 0.001034 | 0.002762 | 0.000942 | 0.000492 | 0.002667 | 0.003593 | 0.000419 | 0.001398 | 0.002508 | 0.000238 |
| extraTrees14 | 0.001532 | 0.003806 | 0.000933 | 0.000844 | 0.002657 | 0.00423 | 0.001866 | 0.000494 | 0.006851 | 0.001371 |
| extraTrees15 | 0.000626 | 0.000562 | 0.000864 | 0 | 0.001274 | 0.001371 | 0.002177 | 0.000287 | 0.002243 | 0.001 |
| extraTrees16 | 0 | 0.00025 | 0.000238 | 0.000238 | 0.00172 | 0.002394 | 0.001366 | 0.001226 | 0.003912 | 0.000369 |
| extraTrees17 | 0.001124 | 0.000472 | 0.001101 | 0.000567 | 0.001567 | 0.003095 | 0.000518 | 0.001739 | 0.003681 | 0.000676 |
| extraTrees18 | 0.001158 | 0.004055 | 0.000959 | 0.005602 | 0.00312 | 0.001161 | 0.000238 | 0.001466 | 0.00233 | 1.26E−18 |
| extraTrees19 | 0.000336 | 0.002558 | 0.003091 | 0.001205 | 0.002679 | 0.001692 | 0.002442 | 0 | 0.001661 | 0.001629 |
| extraTrees20 | 0.001865 | 0.002673 | 0.000415 | 0.006357 | 0.003106 | 0.001863 | 0.002404 | 0 | 0.003564 | 0.001245 |
| extraTrees21 | 0.001136 | 1.23E−18 | 0.001514 | 0.000503 | 0.000984 | 0.001458 | 0.001442 | 0.002068 | 0.001647 | 0.001418 |
| extraTrees22 | 0.002177 | 0.00131 | 0.000492 | 0.004528 | 0.0087 | 0.001051 | 0.00109 | 0.000643 | 0.004415 | 5.26E−05 |
| extraTrees23 | 0.000676 | 0.000781 | 0.001563 | 0.000653 | 0.00479 | 0 | 0.001134 | 0.000909 | 0.001879 | 0 |
| extraTrees24 | 0.001576 | 0.003887 | 0.002771 | 0.001599 | 0.001092 | 0.004554 | 0.00159 | 0.001302 | 0.002762 | 0.000907 |
| extraTrees25 | 0.001405 | 0.001002 | 0.000238 | 0.003915 | 0.00315 | 0.002059 | 5.10E−17 | 0.001358 | 0.001427 | 0.001323 |
| extraTrees26 | 0.002291 | 0.002467 | 0.000388 | 0.001037 | 0.004066 | 0.000948 | 0.000625 | 0.000657 | 0.000777 | 0.001594 |
| extraTrees27 | 0.001034 | 0.002762 | 0.000942 | 0.000492 | 0.002667 | 0.003593 | 0.000419 | 0.001398 | 0.002508 | 0.000238 |
| gbc0 | 0.001074 | 0 | 0 | 2.32E−05 | 0 | 0 | 0 | 3.60E−06 | 0.011546 | 0 |
| gbc1 | 0 | 0 | 0 | 0 | 0.001186 | 0 | 0.000191 | 0 | 0 | 0 |
| gbc2 | 0.000248 | 0 | 5.18E−05 | 0 | 0.004423 | 0.000234 | 0 | 0 | 0 | 0 |
| gbc3 | 0 | 0 | 6.53E−05 | 0 | 0 | 0.020054 | 9.53E−05 | 5.14E−05 | 0 | 0.00726 |
| gbc4 | 0 | 0 | 5.53E−05 | 0.001599 | 1.67E−05 | 2.11E−05 | 0 | 0 | 0 | 0 |
| gbc5 | 0 | 0 | 0.000109 | 0 | 0 | 0 | 0 | 0 | 0 | 0 |
| gbc6 | 0.001152 | 0 | 0 | 0.000675 | 0.001349 | 1.71E−05 | 3.05E−05 | 0.000189 | 0.004095 | 0.000563 |
| gbc7 | 0 | 0.008003 | 0.001381 | 5.35E−05 | 0.00478 | 0 | 0.000255 | 0 | 0 | 0.000518 |
| gbc8 | 7.44E−05 | 0.000491 | 0 | 3.67E−07 | 0 | 4.48E−06 | 0.003772 | 2.09E−06 | 0.012514 | 0 |
| gbc9 | 0.00011 | 0 | 0.000234 | 0 | 1.26E−05 | 0 | 0 | 0.000186 | 0 | 0 |
| gbc10 | 1.71E−06 | 5.19E−05 | 0 | 0.004334 | 0.000463 | 0 | 0.000181 | 0 | 6.42E−05 | 2.89E−05 |
| gbc11 | 0 | 0 | 0 | 0 | 4.54E−05 | 0 | 0 | 0 | 0.000118 | 6.95E−06 |
| gbc12 | 0.00396 | 0 | 4.87E−05 | 0 | 0.003455 | 0 | 0 | 0 | 0 | 0.000293 |
| gbc13 | 0 | 0.0002 | 0 | 7.46E−05 | 0 | 0 | 0 | 0.001622 | 0 | 4.64E−05 |
| gbc14 | 0.001074 | 0 | 0 | 2.32E−05 | 0 | 0 | 0 | 3.60E−06 | 0.011546 | 0 |
| gbc15 | 0 | 0 | 0 | 0 | 0.001186 | 0 | 0.000191 | 0 | 0 | 0 |
| gbc16 | 0.000248 | 0 | 5.18E−05 | 0 | 0.004423 | 0.000234 | 0 | 0 | 0 | 0 |
| gbc17 | 0 | 0 | 6.53E−05 | 0 | 0 | 0.020054 | 9.53E−05 | 5.14E−05 | 0 | 0.00726 |
| gbc18 | 0 | 0 | 5.53E−05 | 0.001599 | 1.67E−05 | 2.11E−05 | 0 | 0 | 0 | 0 |
| gbc19 | 0 | 0 | 0.000109 | 0 | 0 | 0 | 0 | 0 | 0 | 0 |
| gbc20 | 0.001152 | 0 | 0 | 0.000675 | 0.001349 | 1.71E−05 | 3.05E−05 | 0.000189 | 0.004095 | 0.000563 |
| gbc21 | 0 | 0.008003 | 0.001381 | 5.35E−05 | 0.00478 | 0 | 0.000255 | 0 | 0 | 0.000518 |
| gbc22 | 7.44E−05 | 0.000491 | 0 | 3.67E−07 | 0 | 4.48E−06 | 0.003772 | 2.09E−06 | 0.012514 | 0 |
| gbc23 | 0.00011 | 0 | 0.000234 | 0 | 1.26E−05 | 0 | 0 | 0.000186 | 0 | 0 |
| gbc24 | 1.71E−06 | 5.19E−05 | 0 | 0.004334 | 0.000463 | 0 | 0.000181 | 0 | 6.42E−05 | 2.89E−05 |
| gbc25 | 0 | 0 | 0 | 0 | 4.54E−05 | 0 | 0 | 0 | 0.000118 | 6.95E−06 |
| gbc26 | 0.00396 | 0 | 4.87E−05 | 0 | 0.003455 | 0 | 0 | 0 | 0 | 0.000293 |
| gbc27 | 0 | 0.0002 | 0 | 7.46E−05 | 0 | 0 | 0 | 0.001622 | 0 | 4.64E−05 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |
| Gene | RCHY1 | RGCC | RHOC | RIC8A | RIOK2 | RNF138 | RNF24 | RP9 | RPL41 | RPTOR |
| rf0 | 0 | 0 | 0.004253 | 0.000444 | 0 | 0.000325 | 0.002686 | 0.000836 | 0.004885 | 0 |
| rf1 | 0 | 0.005757 | 0.002245 | 0.001907 | 0 | 0.002306 | 0.003552 | 0 | 0.004957 | 0.000713 |
| rf2 | 0 | 0.003392 | 0.002293 | 0 | 0.00211 | 0.004354 | 0 | 0.002772 | 0.001681 | 0.002209 |
| rf3 | 0 | 0.001571 | 8.01E−05 | 0 | 0.002328 | 0 | 0.001043 | 0.000763 | 0.002366 | 0.002037 |
| rf4 | 0 | 0.007902 | 0.00311 | 0.001064 | 0.001772 | 0.004269 | 0 | 0 | 0 | 0.001518 |
| rf5 | 0 | 0.005916 | 0.003786 | 0.00204 | 0.001915 | 0.002548 | 0.001814 | 0.002844 | 1.07E−18 | 2.71E−18 |
| rf6 | 0.005879 | 0.00149 | 0.004723 | 0.002533 | 0.001059 | 0.005877 | 0.001858 | 0 | 0.005113 | 1.05E−18 |
| rf7 | 0.001796 | 0.005025 | 0.001527 | 0.005518 | 0 | 0 | 0.004426 | 5.89E−19 | 0.002359 | 0.001895 |
| rf8 | 0.000386 | 0.001134 | 0.005767 | 0.001705 | 5.34E−18 | 0 | 0 | 0.001087 | 0.000764 | 0.001477 |
| rf9 | 0.002215 | 0.004047 | 0.004002 | 0.000576 | 0.000311 | 0.003003 | 0 | 0 | 0.001249 | 0 |
| rf10 | 0.003923 | 0.001699 | 0.005218 | 0.000772 | 0.001782 | 0.005319 | 0.00271 | 1.15E−18 | 0.003307 | 0.003496 |
| rf11 | 0 | 0 | 0.001763 | 0.001002 | 0 | 0.015139 | 0.001447 | 2.39E−18 | 0.001709 | 0.001759 |
| rf12 | 0.008172 | 0.002441 | 0.004865 | 0.000378 | 0.002845 | 0.003022 | 0 | 0.004054 | 0.007825 | 0 |
| rf13 | 0.001189 | 0.002426 | 0.004755 | 0.000488 | 0 | 0.004121 | 0 | 0.000944 | 0.008924 | 4.19E−18 |
| rf14 | 0 | 0 | 0.004253 | 0.000444 | 0 | 0.000325 | 0.002686 | 0.000836 | 0.004885 | 0 |
| rf15 | 0 | 0.005757 | 0.002245 | 0.001907 | 0 | 0.002306 | 0.003552 | 0 | 0.004957 | 0.000713 |
| rf16 | 0 | 0.003392 | 0.002293 | 0 | 0.00211 | 0.004354 | 0 | 0.002772 | 0.001681 | 0.002209 |
| rf17 | 0 | 0.001571 | 8.01E−05 | 0 | 0.002328 | 0 | 0.001043 | 0.000763 | 0.002366 | 0.002037 |
| rf18 | 0 | 0.007902 | 0.00311 | 0.001064 | 0.001772 | 0.004269 | 0 | 0 | 0 | 0.001518 |
| rf19 | 0 | 0.005916 | 0.003786 | 0.00204 | 0.001915 | 0.002548 | 0.001814 | 0.002844 | 1.07E−18 | 2.71E−18 |
| rf20 | 0.005879 | 0.00149 | 0.004723 | 0.002533 | 0.001059 | 0.005877 | 0.001858 | 0 | 0.005113 | 1.05E−18 |
| rf21 | 0.001796 | 0.005025 | 0.001527 | 0.005518 | 0 | 0 | 0.004426 | 5.89E−19 | 0.002359 | 0.001895 |
| rf22 | 0.000386 | 0.001134 | 0.005767 | 0.001705 | 5.34E−18 | 0 | 0 | 0.001087 | 0.000764 | 0.001477 |
| rf23 | 0.002215 | 0.004047 | 0.004002 | 0.000576 | 0.000311 | 0.003003 | 0 | 0 | 0.001249 | 0 |
| rf24 | 0.003923 | 0.001699 | 0.005218 | 0.000772 | 0.001782 | 0.005319 | 0.00271 | 1.15E−18 | 0.003307 | 0.003496 |
| rf25 | 0 | 0 | 0.001763 | 0.001002 | 0 | 0.015139 | 0.001447 | 2.39E−18 | 0.001709 | 0.001759 |
| rf26 | 0.008172 | 0.002441 | 0.004865 | 0.000378 | 0.002845 | 0.003022 | 0 | 0.004054 | 0.007825 | 0 |
| rf27 | 0.001189 | 0.002426 | 0.004755 | 0.000488 | 0 | 0.004121 | 0 | 0.000944 | 0.008924 | 4.19E−18 |
| extraTrees0 | 0.000911 | 0.00367 | 0.003868 | 0 | 0.002052 | 0.00315 | 0.000559 | 0.000881 | 0.005936 | 0.000511 |
| extraTrees1 | 0.000737 | 0.002396 | 0.002911 | 0.000566 | 2.05E−19 | 0.002034 | 2.17E−18 | 0.000478 | 0.00346 | 0.000238 |
| extraTrees2 | 0.001778 | 0.001684 | 0.004305 | 0.000434 | 0 | 0.003249 | 0.002306 | 0.002347 | 0.005656 | 0.002509 |
| extraTrees3 | 0.000696 | 0.003324 | 5.26E−19 | 0.001659 | 0.001435 | 0.003245 | 0.002128 | 0.00063 | 0.001723 | 0.001242 |
| extraTrees4 | 2.27E−18 | 0.00337 | 0.001892 | 0.002141 | 0.000735 | 0.001506 | 3.66E−05 | 0.002127 | 0.000776 | 0.001117 |
| extraTrees5 | 0.003254 | 0.002124 | 0.002436 | 0.000287 | 0.001547 | 0.000505 | 0.000488 | 0.00026 | 0.00281 | 0.001092 |
| extraTrees6 | 1.26E−18 | 0.000518 | 0.000925 | 0.000293 | 0.000676 | 0.003152 | 0.001407 | 0.002653 | 0.000517 | 0.001265 |
| extraTrees7 | 2.05E−19 | 0.000946 | 0.001981 | 0.001369 | 0.000723 | 0.001406 | 0.001444 | 0.00103 | 0.002378 | 0.002528 |
| extraTrees8 | 0.000508 | 0.006096 | 0.000752 | 0.001807 | 0.003056 | 0 | 0.004509 | 0.000492 | 0.002244 | 0.002735 |
| extraTrees9 | 0.000238 | 0.002813 | 0.00524 | 0.001155 | 0.000876 | 0.003357 | 0.000397 | 0.001282 | 0.002368 | 0.001365 |
| extraTrees10 | 0.001934 | 0.002472 | 0.002675 | 0.001687 | 0.002017 | 0.000551 | 0.002429 | 0.000963 | 0.001996 | 0.000287 |
| extraTrees11 | 0.001851 | 0.001613 | 0.001788 | 0.001612 | 0.001859 | 0.001995 | 0.002746 | 0.001347 | 0.002452 | 0.001218 |
| extraTrees12 | 0.000796 | 0.003696 | 0.000558 | 0.00189 | 0.000242 | 1.06E−18 | 0.002278 | 0.000429 | 0.004207 | 0.00054 |
| extraTrees13 | 0.002859 | 0.002662 | 0.000452 | 0.00067 | 0.000778 | 0.002579 | 0.001859 | 0.000706 | 0.003247 | 0.000754 |
| extraTrees14 | 0.000911 | 0.00367 | 0.003868 | 0 | 0.002052 | 0.00315 | 0.000559 | 0.000881 | 0.005936 | 0.000511 |
| extraTrees15 | 0.000737 | 0.002396 | 0.002911 | 0.000566 | 2.05E−19 | 0.002034 | 2.17E−18 | 0.000478 | 0.00346 | 0.000238 |
| extraTrees16 | 0.001778 | 0.001684 | 0.004305 | 0.000434 | 0 | 0.003249 | 0.002306 | 0.002347 | 0.005656 | 0.002509 |
| extraTrees17 | 0.000696 | 0.003324 | 5.26E−19 | 0.001659 | 0.001435 | 0.003245 | 0.002128 | 0.00063 | 0.001723 | 0.001242 |
| extraTrees18 | 2.27E−18 | 0.00337 | 0.001892 | 0.002141 | 0.000735 | 0.001506 | 3.66E−05 | 0.002127 | 0.000776 | 0.001117 |
| extraTrees19 | 0.003254 | 0.002124 | 0.002436 | 0.000287 | 0.001547 | 0.000505 | 0.000488 | 0.00026 | 0.00281 | 0.001092 |
| extraTrees20 | 1.26E−18 | 0.000518 | 0.000925 | 0.000293 | 0.000676 | 0.003152 | 0.001407 | 0.002653 | 0.000517 | 0.001265 |
| extraTrees21 | 2.05E−19 | 0.000946 | 0.001981 | 0.001369 | 0.000723 | 0.001406 | 0.001444 | 0.00103 | 0.002378 | 0.002528 |
| extraTrees22 | 0.000508 | 0.006096 | 0.000752 | 0.001807 | 0.003056 | 0 | 0.004509 | 0.000492 | 0.002244 | 0.002735 |
| extraTrees23 | 0.000238 | 0.002813 | 0.00524 | 0.001155 | 0.000876 | 0.003357 | 0.000397 | 0.001282 | 0.002368 | 0.001365 |
| extraTrees24 | 0.001934 | 0.002472 | 0.002675 | 0.001687 | 0.002017 | 0.000551 | 0.002429 | 0.000963 | 0.001996 | 0.000287 |
| extraTrees25 | 0.001851 | 0.001613 | 0.001788 | 0.001612 | 0.001859 | 0.001995 | 0.002746 | 0.001347 | 0.002452 | 0.001218 |
| extraTrees26 | 0.000796 | 0.003696 | 0.000558 | 0.00189 | 0.000242 | 1.06E−18 | 0.002278 | 0.000429 | 0.004207 | 0.00054 |
| extraTrees27 | 0.002859 | 0.002662 | 0.000452 | 0.00067 | 0.000778 | 0.002579 | 0.001859 | 0.000706 | 0.003247 | 0.000754 |
| gbc0 | 6.55E−05 | 4.61E−05 | 0 | 0 | 0 | 0.001474 | 0 | 0 | 0 | 0 |
| gbc1 | 0.00259 | 0.000441 | 3.69E−05 | 2.00E−05 | 0 | 0.0004 | 0.000752 | 6.22E−05 | 0 | 0.000948 |
| gbc2 | 1.14E−05 | 0.005629 | 0 | 0.002946 | 0 | 0.006079 | 0 | 0.012769 | 1.70E−05 | 0.002532 |
| gbc3 | 0.002556 | 0.000487 | 0 | 0 | 0.000721 | 0.001451 | 1.18E−07 | 0 | 0.000582 | 0.001225 |
| gbc4 | 0.000155 | 0.001769 | 0 | 0 | 0 | 0 | 0.000572 | 0.015655 | 0 | 0 |
| gbc5 | 0.000432 | 0.023747 | 0 | 0 | 0 | 0.000257 | 0 | 0.0039 | 0 | 0 |
| gbc6 | 0 | 0 | 2.88E−05 | 0 | 0 | 0 | 0 | 0.001042 | 8.99E−09 | 1.21E−05 |
| gbc7 | 0.00079 | 0 | 0 | 0 | 0 | 0.000281 | 1.04E−05 | 0.002627 | 0.000251 | 0 |
| gbc8 | 0.000613 | 0 | 0 | 0 | 0 | 0 | 0 | 0 | 0 | 0.000654 |
| gbc9 | 0 | 5.49E−06 | 6.45E−05 | 0 | 4.19E−05 | 0.000268 | 0.007124 | 0.000203 | 0.001034 | 0.000239 |
| gbc10 | 0 | 0.012375 | 0.021408 | 0.000101 | 0 | 0.001897 | 0 | 3.78E−05 | 0 | 0.003734 |
| gbc11 | 0.002049 | 0 | 0 | 4.30E−06 | 0 | 0 | 0.003823 | 0 | 0.00187 | 0.000701 |
| gbc12 | 0.000521 | 0 | 0.001839 | 0 | 0 | 0 | 0 | 0 | 0.001768 | 7.76E−05 |
| gbc13 | 0 | 0 | 0.000558 | 0 | 0 | 0 | 0.011617 | 0.000279 | 0.000649 | 0.001041 |
| gbc14 | 6.55E−05 | 4.61E−05 | 0 | 0 | 0 | 0.001474 | 0 | 0 | 0 | 0 |
| gbc15 | 0.00259 | 0.000441 | 3.69E−05 | 2.00E−05 | 0 | 0.0004 | 0.000752 | 6.22E−05 | 0 | 0.000948 |
| gbc16 | 1.14E−05 | 0.005629 | 0 | 0.002946 | 0 | 0.006079 | 0 | 0.012769 | 1.70E−05 | 0.002532 |
| gbc17 | 0.002556 | 0.000487 | 0 | 0 | 0.000721 | 0.001451 | 1.18E−07 | 0 | 0.000582 | 0.001225 |
| gbc18 | 0.000155 | 0.001769 | 0 | 0 | 0 | 0 | 0.000572 | 0.015655 | 0 | 0 |
| gbc19 | 0.000432 | 0.023747 | 0 | 0 | 0 | 0.000257 | 0 | 0.0039 | 0 | 0 |
| gbc20 | 0 | 0 | 2.88E−05 | 0 | 0 | 0 | 0 | 0.001042 | 8.99E−09 | 1.21E−05 |
| gbc21 | 0.00079 | 0 | 0 | 0 | 0 | 0.000281 | 1.04E−05 | 0.002627 | 0.000251 | 0 |
| gbc22 | 0.000613 | 0 | 0 | 0 | 0 | 0 | 0 | 0 | 0 | 0.000654 |
| gbc23 | 0 | 5.49E−06 | 6.45E−05 | 0 | 4.19E−05 | 0.000268 | 0.007124 | 0.000203 | 0.001034 | 0.000239 |
| gbc24 | 0 | 0.012375 | 0.021408 | 0.000101 | 0 | 0.001897 | 0 | 3.78E−05 | 0 | 0.003734 |
| gbc25 | 0.002049 | 0 | 0 | 4.30E−06 | 0 | 0 | 0.003823 | 0 | 0.00187 | 0.000701 |
| gbc26 | 0.000521 | 0 | 0.001839 | 0 | 0 | 0 | 0 | 0 | 0.001768 | 7.76E−05 |
| gbc27 | 0 | 0 | 0.000558 | 0 | 0 | 0 | 0.011617 | 0.000279 | 0.000649 | 0.001041 |
| ada0 | 0 | 0.04 | 0 | 0 | 0 | 0 | 0 | 0 | 0 | 0 |
| ada1 | 0 | 0.04 | 0 | 0 | 0 | 0 | 0 | 0 | 0 | 0 |
| ada2 | 0 | 0.04 | 0 | 0 | 0 | 0 | 0 | 0 | 0 | 0 |
| ada3 | 0 | 0.04 | 0 | 0 | 0 | 0 | 0 | 0 | 0 | 0 |
| ada4 | 0 | 0.04 | 0 | 0 | 0 | 0 | 0 | 0 | 0 | 0 |
| ada5 | 0 | 0.04 | 0 | 0 | 0 | 0 | 0 | 0 | 0 | 0 |
| ada6 | 0 | 0.04 | 0 | 0 | 0 | 0 | 0 | 0 | 0 | 0 |
| ada7 | 0 | 0.04 | 0 | 0 | 0 | 0 | 0 | 0 | 0 | 0 |
| ada8 | 0 | 0.04 | 0 | 0 | 0 | 0 | 0 | 0 | 0 | 0 |
| ada9 | 0 | 0.04 | 0 | 0 | 0 | 0 | 0 | 0 | 0 | 0 |
| ada10 | 0 | 0.04 | 0 | 0 | 0 | 0 | 0 | 0 | 0 | 0 |
| ada11 | 0 | 0.04 | 0 | 0 | 0 | 0 | 0 | 0 | 0 | 0 |
| ada12 | 0 | 0.04 | 0 | 0 | 0 | 0 | 0 | 0 | 0 | 0 |
| ada13 | 0 | 0.04 | 0 | 0 | 0 | 0 | 0 | 0 | 0 | 0 |
| ada14 | 0 | 0.04 | 0 | 0 | 0 | 0 | 0 | 0 | 0 | 0 |
| ada15 | 0 | 0.04 | 0 | 0 | 0 | 0 | 0 | 0 | 0 | 0 |
| ada16 | 0 | 0.04 | 0 | 0 | 0 | 0 | 0 | 0 | 0 | 0 |
| ada17 | 0 | 0.04 | 0 | 0 | 0 | 0 | 0 | 0 | 0 | 0 |
| ada18 | 0 | 0.04 | 0 | 0 | 0 | 0 | 0 | 0 | 0 | 0 |
| ada19 | 0 | 0.04 | 0 | 0 | 0 | 0 | 0 | 0 | 0 | 0 |
| ada20 | 0 | 0.04 | 0 | 0 | 0 | 0 | 0 | 0 | 0 | 0 |
| ada21 | 0 | 0.04 | 0 | 0 | 0 | 0 | 0 | 0 | 0 | 0 |
| ada22 | 0 | 0.04 | 0 | 0 | 0 | 0 | 0 | 0 | 0 | 0 |
| ada23 | 0 | 0.04 | 0 | 0 | 0 | 0 | 0 | 0 | 0 | 0 |
| ada24 | 0 | 0.04 | 0 | 0 | 0 | 0 | 0 | 0 | 0 | 0 |
| ada25 | 0 | 0.04 | 0 | 0 | 0 | 0 | 0 | 0 | 0 | 0 |
| ada26 | 0 | 0.04 | 0 | 0 | 0 | 0 | 0 | 0 | 0 | 0 |
| ada27 | 0 | 0.04 | 0 | 0 | 0 | 0 | 0 | 0 | 0 | 0 |
| Gene | RRP15 | SAR1B | SCAMP1 | SCFD1 | SEC14L2 | SFT2D1 | SFXN5 | SGO2 | SGTB | SH3BP4 |
| rf0 | 0 | 0.013103 | 0 | 0.006213 | 0.000366 | 0.006519 | 0 | 0.004852 | 0 | 0.000362 |
| rf1 | 0 | 0.002923 | 0.000367 | 0.003998 | 0.001817 | 4.62E−18 | 4.96E−19 | 0.003179 | 0.000359 | 0.004505 |
| rf2 | 0.001707 | 0.003577 | 0.001007 | 0.006898 | 0 | 0 | 0.000695 | 0.000354 | 0.003626 | 0.000949 |
| rf3 | 0 | 0.014035 | 0.00858 | 0.008296 | 0.001414 | 0.003651 | 0.005634 | 0.006762 | 0.000424 | 0.001217 |
| rf4 | 0 | 0.000921 | 0.001983 | 0.001032 | 0.000838 | 0.000723 | 0.004459 | 0.001831 | 0.001282 | 0 |
| rf5 | 0 | 0.001857 | 0 | 0.007103 | 0 | 1.03E−18 | 0.006097 | 0.005435 | 0 | 0.000471 |
| rf6 | 0.0033 | 0.001287 | 0 | 0.007783 | 0.000376 | 0.001425 | 0.008816 | 0.000732 | 0 | 0 |
| rf7 | 0.001987 | 0.007647 | 0.002056 | 0.002183 | 0.002862 | 0.00109 | 0.016202 | 0.008268 | 0.00564 | 0.000898 |
| rf8 | 0 | 0.005861 | 0.002465 | 0.00229 | 0.0023 | 0 | 0.001384 | 0.007926 | 0.003648 | 0.00578 |
| rf9 | 0.00263 | 0.005732 | 0.003728 | 2.14E−17 | 0.000489 | 0.001766 | 0.007263 | 0.001413 | 0.007159 | 0.001863 |
| rf10 | 0.012585 | 0.008185 | 0.002489 | 0.002549 | 0.000427 | 0.004699 | 0.000443 | 0.002291 | 0.000349 | 0.0018 |
| rf11 | 0.001023 | 0.003742 | 0.005069 | 0.000713 | 0 | 0.002242 | 0.001186 | 0.004372 | 1.76E−18 | 0 |
| rf12 | 0.003873 | 0.012735 | 0.002608 | 0.006979 | 0.00139 | 0.000816 | 0.0095 | 0.004593 | 3.36E−19 | 0.004742 |
| rf13 | 4.22E−18 | 0.006292 | 0.001981 | 0.002014 | 0.002347 | 0.008064 | 0 | 0 | 0.001854 | 0.007862 |
| rf14 | 0 | 0.013103 | 0 | 0.006213 | 0.000366 | 0.006519 | 0 | 0.004852 | 0 | 0.000362 |
| rf15 | 0 | 0.002923 | 0.000367 | 0.003998 | 0.001817 | 4.62E−18 | 4.96E−19 | 0.003179 | 0.000359 | 0.004505 |
| rf16 | 0.001707 | 0.003577 | 0.001007 | 0.006898 | 0 | 0 | 0.000695 | 0.000354 | 0.003626 | 0.000949 |
| rf17 | 0 | 0.014035 | 0.00858 | 0.008296 | 0.001414 | 0.003651 | 0.005634 | 0.006762 | 0.000424 | 0.001217 |
| rf18 | 0 | 0.000921 | 0.001983 | 0.001032 | 0.000838 | 0.000723 | 0.004459 | 0.001831 | 0.001282 | 0 |
| rf19 | 0 | 0.001857 | 0 | 0.007103 | 0 | 1.03E−18 | 0.006097 | 0.005435 | 0 | 0.000471 |
| rf20 | 0.0033 | 0.001287 | 0 | 0.007783 | 0.000376 | 0.001425 | 0.008816 | 0.000732 | 0 | 0 |
| rf21 | 0.001987 | 0.007647 | 0.002056 | 0.002183 | 0.002862 | 0.00109 | 0.016202 | 0.008268 | 0.00564 | 0.000898 |
| rf22 | 0 | 0.005861 | 0.002465 | 0.00229 | 0.0023 | 0 | 0.001384 | 0.007926 | 0.003648 | 0.00578 |
| rf23 | 0.00263 | 0.005732 | 0.003728 | 2.14E−17 | 0.000489 | 0.001766 | 0.007263 | 0.001413 | 0.007159 | 0.001863 |
| rf24 | 0.012585 | 0.008185 | 0.002489 | 0.002549 | 0.000427 | 0.004699 | 0.000443 | 0.002291 | 0.000349 | 0.0018 |
| rf25 | 0.001023 | 0.003742 | 0.005069 | 0.000713 | 0 | 0.002242 | 0.001186 | 0.004372 | 1.76E−18 | 0 |
| rf26 | 0.003873 | 0.012735 | 0.002608 | 0.006979 | 0.00139 | 0.000816 | 0.0095 | 0.004593 | 3.36E−19 | 0.004742 |
| rf27 | 4.22E−18 | 0.006292 | 0.001981 | 0.002014 | 0.002347 | 0.008064 | 0 | 0 | 0.001854 | 0.007862 |
| extraTrees0 | 7.01E−19 | 0.004331 | 0.000295 | 0.002499 | 0.001562 | 0.003847 | 0.003682 | 0.003919 | 0.001096 | 0.004201 |
| extraTrees1 | 0.000409 | 0.003486 | 0.00265 | 0.000252 | 0 | 0.000873 | 0.002326 | 0.002847 | 0.000669 | 0.002699 |
| extraTrees2 | 0.000884 | 0.00151 | 0 | 0.003148 | 0.001487 | 0.001304 | 0.006629 | 0.002905 | 0.000276 | 0.001849 |
| extraTrees3 | 0.001031 | 0.003778 | 0.001449 | 0.000773 | 0.000473 | 0.002508 | 0.00327 | 0.003043 | 0.002373 | 0.001033 |
| extraTrees4 | 5.26E−19 | 0.001552 | 0.001613 | 0.001426 | 0.000238 | 0.002708 | 0.004445 | 0.002054 | 0.002571 | 0.00145 |
| extraTrees5 | 0.001674 | 0.005146 | 0.00194 | 0.00169 | 0 | 0.004794 | 0.001942 | 0.004262 | 0.001717 | 0.003618 |
| extraTrees6 | 0.000861 | 3.51E−19 | 0.002224 | 0.001209 | 0.001595 | 0.003698 | 0.002681 | 0.00171 | 0.001266 | 0.004154 |
| extraTrees7 | 0.001311 | 0.001959 | 0 | 0.000267 | 0.000871 | 0.001223 | 0.00386 | 0.00186 | 2.62E−18 | 0.002583 |
| extraTrees8 | 0.000507 | 0.002844 | 0.001804 | 0.00059 | 0.000301 | 0.003464 | 0.002875 | 0.001506 | 0.00108 | 0.004156 |
| extraTrees9 | 0.00179 | 0.003994 | 0.002195 | 0.002443 | 0.000238 | 0.00194 | 0.003303 | 0.001027 | 0.002705 | 0.003296 |
| extraTrees10 | 0.000924 | 0.003668 | 5.26E−05 | 0.001049 | 0.000254 | 0.003456 | 0.004395 | 0.001524 | 0.000548 | 0.003129 |
| extraTrees11 | 0.001154 | 0.00912 | 0.000498 | 0.00026 | 0.002916 | 0.001733 | 0.000653 | 0.003968 | 0.001639 | 0.001692 |
| extraTrees12 | 0.001792 | 0.00438 | 0.002654 | 0.003089 | 0.000966 | 0.002531 | 0 | 0.00101 | 0.002209 | 0.004564 |
| extraTrees13 | 0.003343 | 0.001991 | 0.000508 | 0.003145 | 0.000659 | 0.00222 | 0.001164 | 0.002952 | 0.000238 | 0.0035 |
| extraTrees14 | 7.01E−19 | 0.004331 | 0.000295 | 0.002499 | 0.001562 | 0.003847 | 0.003682 | 0.003919 | 0.001096 | 0.004201 |
| extraTrees15 | 0.000409 | 0.003486 | 0.00265 | 0.000252 | 0 | 0.000873 | 0.002326 | 0.002847 | 0.000669 | 0.002699 |
| extraTrees16 | 0.000884 | 0.00151 | 0 | 0.003148 | 0.001487 | 0.001304 | 0.006629 | 0.002905 | 0.000276 | 0.001849 |
| extraTrees17 | 0.001031 | 0.003778 | 0.001449 | 0.000773 | 0.000473 | 0.002508 | 0.00327 | 0.003043 | 0.002373 | 0.001033 |
| extraTrees18 | 5.26E−19 | 0.001552 | 0.001613 | 0.001426 | 0.000238 | 0.002708 | 0.004445 | 0.002054 | 0.002571 | 0.00145 |
| extraTrees19 | 0.001674 | 0.005146 | 0.00194 | 0.00169 | 0 | 0.004794 | 0.001942 | 0.004262 | 0.001717 | 0.003618 |
| extraTrees20 | 0.000861 | 3.51E−19 | 0.002224 | 0.001209 | 0.001595 | 0.003698 | 0.002681 | 0.00171 | 0.001266 | 0.004154 |
| extraTrees21 | 0.001311 | 0.001959 | 0 | 0.000267 | 0.000871 | 0.001223 | 0.00386 | 0.00186 | 2.62E−18 | 0.002583 |
| extraTrees22 | 0.000507 | 0.002844 | 0.001804 | 0.00059 | 0.000301 | 0.003464 | 0.002875 | 0.001506 | 0.00108 | 0.004156 |
| extraTrees23 | 0.00179 | 0.003994 | 0.002195 | 0.002443 | 0.000238 | 0.00194 | 0.003303 | 0.001027 | 0.002705 | 0.003296 |
| extraTrees24 | 0.000924 | 0.003668 | 5.26E−05 | 0.001049 | 0.000254 | 0.003456 | 0.004395 | 0.001524 | 0.000548 | 0.003129 |
| extraTrees25 | 0.001154 | 0.00912 | 0.000498 | 0.00026 | 0.002916 | 0.001733 | 0.000653 | 0.003968 | 0.001639 | 0.001692 |
| extraTrees26 | 0.001792 | 0.00438 | 0.002654 | 0.003089 | 0.000966 | 0.002531 | 0 | 0.00101 | 0.002209 | 0.004564 |
| extraTrees27 | 0.003343 | 0.001991 | 0.000508 | 0.003145 | 0.000659 | 0.00222 | 0.001164 | 0.002952 | 0.000238 | 0.0035 |
| gbc0 | 0 | 0 | 0.000254 | 0.000636 | 0 | 0.003813 | 0 | 4.60E−05 | 0 | 0.000726 |
| gbc1 | 0.02533 | 0.000404 | 0 | 0.019729 | 0 | 0 | 0 | 0.000176 | 2.24E−05 | 0 |
| gbc2 | 0 | 0.000708 | 7.54E−06 | 0 | 9.58E−06 | 0 | 0 | 0.000404 | 0.00013 | 0.019 |
| gbc3 | 0.005546 | 0.024996 | 0.003382 | 0.00105 | 0.003195 | 0 | 0 | 0 | 0 | 0 |
| gbc4 | 0 | 0.000264 | 0.001878 | 0 | 0 | 0.001154 | 0 | 0.000151 | 0 | 0 |
| gbc5 | 0.004389 | 0.000315 | 1.13E−05 | 0.005269 | 0 | 0.003124 | 0 | 0.000225 | 0 | 0 |
| gbc6 | 0 | 0.016394 | 0 | 1.26E−05 | 0 | 0.000254 | 0 | 0 | 2.04E−05 | 0.003413 |
| gbc7 | 0 | 0.001242 | 0 | 0.016151 | 3.69E−05 | 0.000343 | 0.003492 | 0.000734 | 0.000911 | 0 |
| gbc8 | 0 | 0.027058 | 0 | 1.95E−05 | 0 | 0 | 6.02E−05 | 2.48E−05 | 0 | 0 |
| gbc9 | 0 | 0 | 1.28E−06 | 0.006257 | 2.38E−05 | 0 | 0.013573 | 0.000124 | 0 | 0 |
| gbc10 | 2.38E−05 | 0 | 0 | 0.015844 | 1.21E−05 | 0.004145 | 0 | 0 | 0.003232 | 0.010725 |
| gbc11 | 4.93E−05 | 0.006869 | 0 | 0 | 0.000307 | 0.000242 | 0.00495 | 0.003665 | 0 | 0 |
| gbc12 | 0.005549 | 0.012269 | 0 | 0.006683 | 0.005656 | 0 | 1.53E−05 | 0.000509 | 0 | 0 |
| gbc13 | 0 | 0.015559 | 0 | 0.020523 | 0 | 0 | 0 | 0.008465 | 0 | 0.000142 |
| gbc14 | 0 | 0 | 0.000254 | 0.000636 | 0 | 0.003813 | 0 | 4.60E−05 | 0 | 0.000726 |
| gbc15 | 0.02533 | 0.000404 | 0 | 0.019729 | 0 | 0 | 0 | 0.000176 | 2.24E−05 | 0 |
| gbc16 | 0 | 0.000708 | 7.54E−06 | 0 | 9.58E−06 | 0 | 0 | 0.000404 | 0.00013 | 0.019 |
| gbc17 | 0.005546 | 0.024996 | 0.003382 | 0.00105 | 0.003195 | 0 | 0 | 0 | 0 | 0 |
| gbc18 | 0 | 0.000264 | 0.001878 | 0 | 0 | 0.001154 | 0 | 0.000151 | 0 | 0 |
| gbc19 | 0.004389 | 0.000315 | 1.13E−05 | 0.005269 | 0 | 0.003124 | 0 | 0.000225 | 0 | 0 |
| gbc20 | 0 | 0.016394 | 0 | 1.26E−05 | 0 | 0.000254 | 0 | 0 | 2.04E−05 | 0.003413 |
| gbc21 | 0 | 0.001242 | 0 | 0.016151 | 3.69E−05 | 0.000343 | 0.003492 | 0.000734 | 0.000911 | 0 |
| gbc22 | 0 | 0.027058 | 0 | 1.95E−05 | 0 | 0 | 6.02E−05 | 2.48E−05 | 0 | 0 |
| gbc23 | 0 | 0 | 1.28E−06 | 0.006257 | 2.38E−05 | 0 | 0.013573 | 0.000124 | 0 | 0 |
| gbc24 | 2.38E−05 | 0 | 0 | 0.015844 | 1.21E−05 | 0.004145 | 0 | 0 | 0.003232 | 0.010725 |
| gbc25 | 4.93E−05 | 0.006869 | 0 | 0 | 0.000307 | 0.000242 | 0.00495 | 0.003665 | 0 | 0 |
| gbc26 | 0.005549 | 0.012269 | 0 | 0.006683 | 0.005656 | 0 | 1.53E−05 | 0.000509 | 0 | 0 |
| gbc27 | 0 | 0.015559 | 0 | 0.020523 | 0 | 0 | 0 | 0.008465 | 0 | 0.000142 |
| ada0 | 0 | 0 | 0 | 0.02 | 0 | 0 | 0 | 0 | 0 | 0.02 |
| ada1 | 0 | 0 | 0 | 0.02 | 0 | 0 | 0 | 0 | 0 | 0.02 |
| ada2 | 0 | 0 | 0 | 0.02 | 0 | 0 | 0 | 0 | 0 | 0.02 |
| ada3 | 0 | 0 | 0 | 0.02 | 0 | 0 | 0 | 0 | 0 | 0.02 |
| ada4 | 0 | 0 | 0 | 0.02 | 0 | 0 | 0 | 0 | 0 | 0.02 |
| ada5 | 0 | 0 | 0 | 0.02 | 0 | 0 | 0 | 0 | 0 | 0.02 |
| ada6 | 0 | 0 | 0 | 0.02 | 0 | 0 | 0 | 0 | 0 | 0.02 |
| ada7 | 0 | 0 | 0 | 0.02 | 0 | 0 | 0 | 0 | 0 | 0.02 |
| ada8 | 0 | 0 | 0 | 0.02 | 0 | 0 | 0 | 0 | 0 | 0.02 |
| ada9 | 0 | 0 | 0 | 0.02 | 0 | 0 | 0 | 0 | 0 | 0.02 |
| ada10 | 0 | 0 | 0 | 0.02 | 0 | 0 | 0 | 0 | 0 | 0.02 |
| ada11 | 0 | 0 | 0 | 0.02 | 0 | 0 | 0 | 0 | 0 | 0.02 |
| ada12 | 0 | 0 | 0 | 0.02 | 0 | 0 | 0 | 0 | 0 | 0.02 |
| ada13 | 0 | 0 | 0 | 0.02 | 0 | 0 | 0 | 0 | 0 | 0.02 |
| ada14 | 0 | 0 | 0 | 0.02 | 0 | 0 | 0 | 0 | 0 | 0.02 |
| ada15 | 0 | 0 | 0 | 0.02 | 0 | 0 | 0 | 0 | 0 | 0.02 |
| ada16 | 0 | 0 | 0 | 0.02 | 0 | 0 | 0 | 0 | 0 | 0.02 |
| ada17 | 0 | 0 | 0 | 0.02 | 0 | 0 | 0 | 0 | 0 | 0.02 |
| ada18 | 0 | 0 | 0 | 0.02 | 0 | 0 | 0 | 0 | 0 | 0.02 |
| ada19 | 0 | 0 | 0 | 0.02 | 0 | 0 | 0 | 0 | 0 | 0.02 |
| ada20 | 0 | 0 | 0 | 0.02 | 0 | 0 | 0 | 0 | 0 | 0.02 |
| ada21 | 0 | 0 | 0 | 0.02 | 0 | 0 | 0 | 0 | 0 | 0.02 |
| ada22 | 0 | 0 | 0 | 0.02 | 0 | 0 | 0 | 0 | 0 | 0.02 |
| ada23 | 0 | 0 | 0 | 0.02 | 0 | 0 | 0 | 0 | 0 | 0.02 |
| ada24 | 0 | 0 | 0 | 0.02 | 0 | 0 | 0 | 0 | 0 | 0.02 |
| ada25 | 0 | 0 | 0 | 0.02 | 0 | 0 | 0 | 0 | 0 | 0.02 |
| ada26 | 0 | 0 | 0 | 0.02 | 0 | 0 | 0 | 0 | 0 | 0.02 |
| ada27 | 0 | 0 | 0 | 0.02 | 0 | 0 | 0 | 0 | 0 | 0.02 |

| Gene | SLC22A17 | SLC25A24 | SLC35B3 | SLFN14 | SLU7 | SMAD5 | SMARCAD1 | SMC2 | SMC6 | SMIM10L1 |
|---|---|---|---|---|---|---|---|---|---|---|
| rf0 | 0.000678 | 0 | 0.000615 | 4.26E−18 | 0 | 0.004054 | 0.006254 | 0.008564 | 0.00702 | 0.000971 |
| rf1 | 0 | 0.0013 | 0.004301 | 0.003317 | 0 | 0.001531 | 0.002232 | 0.004564 | 0 | 0.004343 |
| rf2 | 0 | 0.000931 | 0.003234 | 0 | 0 | 0 | 0.005729 | 0.008219 | 0.001169 | 0.003088 |
| rf3 | 4.55E−18 | 0.005542 | 0.003835 | 0 | 0 | 0.001037 | 0.000378 | 8.60E−19 | 0.003007 | 0.000725 |
| rf4 | 0.001006 | 0.00397 | 0.00158 | 0 | 0.005296 | 0.003606 | 0.009302 | 0.004933 | 0.001542 | 0.000329 |
| rf5 | 0.002585 | 0.000615 | 0.004052 | 0.001127 | 8.64E−18 | 0.005081 | 0.005717 | 0.005218 | 1.56E−18 | 0 |
| rf6 | 0 | 0 | 0.000469 | 1.45E−18 | 0 | 0.002736 | 0.001311 | 0.008323 | 0.005587 | 0 |
| rf7 | 0.003039 | 0 | 0.00101 | 0 | 0.003016 | 0.003023 | 0.000382 | 0.003778 | 0.003442 | 0 |
| rf8 | 0.000515 | 0.000859 | 0 | 0.003969 | 0 | 0.007115 | 0.002907 | 0.005382 | 0.001846 | 0 |
| rf9 | 0.001296 | 0 | 0.003361 | 0 | 0.001118 | 0.005167 | 0.000829 | 0.001876 | 0.006847 | 0 |
| rf10 | 0.00458 | 0.003452 | 0.004792 | 0.008336 | 0 | 1.11E−18 | 0.005802 | 0.002447 | 0.002558 | 0.000623 |
| rf11 | 0.002267 | 0.001709 | 0.001863 | 2.47E−18 | 0 | 0.006046 | 0.007995 | 0 | 0.000731 | 1.91E−18 |
| rf12 | 0 | 0.004914 | 0.008086 | 0.001067 | 0 | 0.004416 | 0.00829 | 0.002472 | 0.001067 | 0 |
| rf13 | 0 | 0.003457 | 0.007034 | 0 | 0 | 0 | 0.007456 | 0 | 0.002263 | 0 |
| rf14 | 0.000678 | 0 | 0.000615 | 4.26E−18 | 0 | 0.004054 | 0.006254 | 0.008564 | 0.00702 | 0.000971 |
| rf15 | 0 | 0.0013 | 0.004301 | 0.003317 | 0 | 0.001531 | 0.002232 | 0.004564 | 0 | 0.004343 |
| rf16 | 0 | 0.000931 | 0.003234 | 0 | 0 | 0 | 0.005729 | 0.008219 | 0.001169 | 0.003088 |
| rf17 | 4.55E−18 | 0.005542 | 0.003835 | 0 | 0 | 0.001037 | 0.000378 | 8.60E−19 | 0.003007 | 0.000725 |
| rf18 | 0.001006 | 0.00397 | 0.00158 | 0 | 0.005296 | 0.003606 | 0.009302 | 0.004933 | 0.001542 | 0.000329 |
| rf19 | 0.002585 | 0.000615 | 0.004052 | 0.001127 | 8.64E−18 | 0.005081 | 0.005717 | 0.005218 | 1.56E−18 | 0 |
| rf20 | 0 | 0 | 0.000469 | 1.45E−18 | 0 | 0.002736 | 0.001311 | 0.008323 | 0.005587 | 0 |
| rf21 | 0.003039 | 0 | 0.00101 | 0 | 0.003016 | 0.003023 | 0.000382 | 0.003778 | 0.003442 | 0 |
| rf22 | 0.000515 | 0.000859 | 0 | 0.003969 | 0 | 0.007115 | 0.002907 | 0.005382 | 0.001846 | 0 |
| rf23 | 0.001296 | 0 | 0.003361 | 0 | 0.001118 | 0.005167 | 0.000829 | 0.001876 | 0.006847 | 0 |
| rf24 | 0.00458 | 0.003452 | 0.004792 | 0.008336 | 0 | 1.11E−18 | 0.005802 | 0.002447 | 0.002558 | 0.000623 |
| rf25 | 0.002267 | 0.001709 | 0.001863 | 2.47E−18 | 0 | 0.006046 | 0.007995 | 0 | 0.000731 | 1.91E−18 |
| rf26 | 0 | 0.004914 | 0.008086 | 0.001067 | 0 | 0.004416 | 0.00829 | 0.002472 | 0.001067 | 0 |
| rf27 | 0 | 0.003457 | 0.007034 | 0 | 0 | 0 | 0.007456 | 0 | 0.002263 | 0 |
| extraTrees0 | 0.001465 | 0.000527 | 0.00501 | 0 | 0.000625 | 0.005423 | 0.005076 | 0.00305 | 0.003892 | 0.000115 |
| extraTrees1 | 0.002044 | 0.000641 | 0.00151 | 0.002548 | 0.000107 | 0.004903 | 0.006051 | 0.000798 | 4.10E−19 | 0.00028 |
| extraTrees2 | 0.002457 | 0.00127 | 0.00631 | 0.002133 | 1.58E−18 | 0.005187 | 0.009204 | 0.002139 | 0.001508 | 0.000574 |
| extraTrees3 | 7.41E−17 | 0.001514 | 0.004688 | 0.001441 | 0.00242 | 0.00209 | 0.008478 | 0.003678 | 0.003723 | 0 |
| extraTrees4 | 0.000763 | 0.00109 | 0.002019 | 0.000737 | 0.001059 | 0.002743 | 0.003288 | 0.005313 | 0.000602 | 0.002682 |
| extraTrees5 | 0.000234 | 1.23E−18 | 0.002018 | 0.000885 | 0.000476 | 0.001599 | 0.011494 | 0.002416 | 0.002129 | 0.001868 |
| extraTrees6 | 0.000963 | 0.00086 | 0.002189 | 0.001126 | 0.00082 | 0.003472 | 0.007172 | 0.004407 | 3.05E−05 | 0.000676 |
| extraTrees7 | 0.000714 | 0.000753 | 0.004365 | 0.000688 | 0.001818 | 0.002747 | 0.002562 | 0.001964 | 0.002039 | 0.001195 |
| extraTrees8 | 0.001494 | 0.000635 | 0.007473 | 0.000898 | 0.000505 | 0.003772 | 0.005182 | 0.003071 | 0.001742 | 0.000803 |
| extraTrees9 | 0.00228 | 0.001365 | 0.003476 | 0.001257 | 0.001346 | 0.003897 | 0.008657 | 0.005612 | 0.00155 | 0.000737 |
| extraTrees10 | 0.000492 | 0.001085 | 0.004459 | 0.000492 | 5.39E−17 | 0.005949 | 0.005324 | 0.001676 | 0.001493 | 0.001613 |
| extraTrees11 | 0.001762 | 0.001862 | 0.002983 | 0.002101 | 0.000238 | 0.00161 | 0.00164 | 0.00268 | 0.002473 | 0.001085 |
| extraTrees12 | 0.000812 | 2.73E−19 | 0.002526 | 7.41E−17 | 0.000289 | 0.003232 | 0.012546 | 0.004447 | 0.00171 | 0.001786 |
| extraTrees13 | 0.002448 | 0.000905 | 0.003958 | 0.002148 | 0 | 0.004368 | 0.004511 | 0.002408 | 0.003078 | 0.001566 |
| extraTrees14 | 0.001465 | 0.000527 | 0.00501 | 0 | 0.000625 | 0.005423 | 0.005076 | 0.00305 | 0.003892 | 0.000115 |
| extraTrees15 | 0.002044 | 0.000641 | 0.00151 | 0.002548 | 0.000107 | 0.004903 | 0.006051 | 0.000798 | 4.10E−19 | 0.00028 |
| extraTrees16 | 0.002457 | 0.00127 | 0.00631 | 0.002133 | 1.58E−18 | 0.005187 | 0.009204 | 0.002139 | 0.001508 | 0.000574 |
| extraTrees17 | 7.41E−17 | 0.001514 | 0.004688 | 0.001441 | 0.00242 | 0.00209 | 0.008478 | 0.003678 | 0.003723 | 0 |
| extraTrees18 | 0.000763 | 0.00109 | 0.002019 | 0.000737 | 0.001059 | 0.002743 | 0.003288 | 0.005313 | 0.000602 | 0.002682 |
| extraTrees19 | 0.000234 | 1.23E−18 | 0.002018 | 0.000885 | 0.000476 | 0.001599 | 0.011494 | 0.002416 | 0.002129 | 0.001868 |
| extraTrees20 | 0.000963 | 0.00086 | 0.002189 | 0.001126 | 0.00082 | 0.003472 | 0.007172 | 0.004407 | 3.05E−05 | 0.000676 |
| extraTrees21 | 0.000714 | 0.000753 | 0.004365 | 0.000688 | 0.001818 | 0.002747 | 0.002562 | 0.001964 | 0.002039 | 0.001195 |
| extraTrees22 | 0.001494 | 0.000635 | 0.007473 | 0.000898 | 0.000505 | 0.003772 | 0.005182 | 0.003071 | 0.001742 | 0.000803 |
| extraTrees23 | 0.00228 | 0.001365 | 0.003476 | 0.001257 | 0.001346 | 0.003897 | 0.008657 | 0.005612 | 0.00155 | 0.000737 |
| extraTrees24 | 0.000492 | 0.001085 | 0.004459 | 0.000492 | 5.39E−17 | 0.005949 | 0.005324 | 0.001676 | 0.001493 | 0.001613 |
| extraTrees25 | 0.001762 | 0.001862 | 0.002983 | 0.002101 | 0.000238 | 0.00161 | 0.00164 | 0.00268 | 0.002473 | 0.001085 |
| extraTrees26 | 0.000812 | 2.73E−19 | 0.002526 | 7.41E−17 | 0.000289 | 0.003232 | 0.012546 | 0.004447 | 0.00171 | 0.001786 |
| extraTrees27 | 0.002448 | 0.000905 | 0.003958 | 0.002148 | 0 | 0.004368 | 0.004511 | 0.002408 | 0.003078 | 0.001566 |
| gbc0 | 0 | 0.000645 | 0.001023 | 0 | 0.000101 | 0.000443 | 8.10E−05 | 0.022333 | 0.002168 | 0 |
| gbc1 | 9.16E−08 | 0 | 0 | 2.74E−06 | 0 | 0.00053 | 0.007846 | 9.66E−05 | 0.000292 | 0.00077 |
| gbc2 | 0.009818 | 0.000107 | 3.60E−07 | 0 | 0 | 0 | 0 | 0.00685 | 0 | 1.82E−07 |
| gbc3 | 0 | 0 | 0.000293 | 0 | 0 | 0.011754 | 0.003317 | 3.46E−05 | 0.018431 | 0.01202 |
| gbc4 | 6.75E−06 | 0.004225 | 0 | 0 | 0.000445 | 0.000142 | 0.000115 | 3.37E−05 | 0 | 0.000586 |
| gbc5 | 0 | 0.001562 | 0 | 0 | 0.000471 | 0.009804 | 0.002951 | 0.001402 | 0.02025 | 0.001174 |
| gbc6 | 0 | 1.17E−05 | 0.00224 | 0 | 0.001905 | 0.000452 | 0.000132 | 0.00069 | 0 | 0 |
| gbc7 | 0 | 0.017513 | 2.40E−05 | 0 | 0 | 0.008486 | 5.62E−05 | 0.029493 | 0.032944 | 0 |
| gbc8 | 0 | 4.24E−05 | 7.85E−06 | 0 | 0 | 0.000127 | 0.020206 | 0.002353 | 0.018796 | 0 |
| gbc9 | 0 | 4.12E−05 | 0 | 0 | 0 | 0.026926 | 0.010076 | 0.000365 | 0.00762 | 0 |
| gbc10 | 0.009666 | 0.000758 | 0 | 0.001441 | 0 | 0.000183 | 0.003476 | 0.003881 | 2.09E−05 | 4.95E−06 |
| gbc11 | 0 | 0.000414 | 0.001941 | 0 | 6.12E−05 | 0.000458 | 0.006605 | 0.000385 | 0 | 0 |
| gbc12 | 8.97E−08 | 0 | 0 | 0 | 9.33E−06 | 5.89E−05 | 0.001105 | 0.000211 | 0.016645 | 0 |
| gbc13 | 0 | 0 | 0 | 0.025186 | 0.000198 | 0.007737 | 0.04422 | 0.025203 | 0.01495 | 0 |
| gbc14 | 0 | 0.000645 | 0.001023 | 0 | 0.000101 | 0.000443 | 8.10E−05 | 0.022333 | 0.002168 | 0 |
| gbc15 | 9.16E−08 | 0 | 0 | 2.74E−06 | 0 | 0.00053 | 0.007846 | 9.66E−05 | 0.000292 | 0.00077 |
| gbc16 | 0.009818 | 0.000107 | 3.60E−07 | 0 | 0 | 0 | 0 | 0.00685 | 0 | 1.82E−07 |
| gbc17 | 0 | 0 | 0.000293 | 0 | 0 | 0.011754 | 0.003317 | 3.46E−05 | 0.018431 | 0.01202 |
| gbc18 | 6.75E−06 | 0.004225 | 0 | 0 | 0.000445 | 0.000142 | 0.000115 | 3.37E−05 | 0 | 0.000586 |
| gbc19 | 0 | 0.001562 | 0 | 0 | 0.000471 | 0.009804 | 0.002951 | 0.001402 | 0.02025 | 0.001174 |
| gbc20 | 0 | 1.17E−05 | 0.00224 | 0 | 0.001905 | 0.000452 | 0.000132 | 0.00069 | 0 | 0 |
| gbc21 | 0 | 0.017513 | 2.40E−05 | 0 | 0 | 0.008486 | 5.62E−05 | 0.029493 | 0.032944 | 0 |
| gbc22 | 0 | 4.24E−05 | 7.85E−06 | 0 | 0 | 0.000127 | 0.020206 | 0.002353 | 0.018796 | 0 |
| gbc23 | 0 | 4.12E−05 | 0 | 0 | 0 | 0.026926 | 0.010076 | 0.000365 | 0.00762 | 0 |
| gbc24 | 0.009666 | 0.000758 | 0 | 0.001441 | 0 | 0.000183 | 0.003476 | 0.003881 | 2.09E−05 | 4.95E−06 |
| gbc25 | 0 | 0.000414 | 0.001941 | 0 | 6.12E−05 | 0.000458 | 0.006605 | 0.000385 | 0 | 0 |
| gbc26 | 8.97E−08 | 0 | 0 | 0 | 9.33E−06 | 5.89E−05 | 0.001105 | 0.000211 | 0.016645 | 0 |
| gbc27 | 0 | 0 | 0 | 0.025186 | 0.000198 | 0.007737 | 0.04422 | 0.025203 | 0.01495 | 0 |
| ada0 | 0 | 0 | 0 | 0 | 0 | 0 | 0.02 | 0 | 0 | 0 |
| ada1 | 0 | 0 | 0 | 0 | 0 | 0 | 0.02 | 0 | 0 | 0 |
| ada2 | 0 | 0 | 0 | 0 | 0 | 0 | 0.02 | 0 | 0 | 0 |
| ada3 | 0 | 0 | 0 | 0 | 0 | 0 | 0.02 | 0 | 0 | 0 |
| ada4 | 0 | 0 | 0 | 0 | 0 | 0 | 0.02 | 0 | 0 | 0 |
| ada5 | 0 | 0 | 0 | 0 | 0 | 0 | 0.02 | 0 | 0 | 0 |
| ada6 | 0 | 0 | 0 | 0 | 0 | 0 | 0.02 | 0 | 0 | 0 |
| ada7 | 0 | 0 | 0 | 0 | 0 | 0 | 0.02 | 0 | 0 | 0 |
| ada8 | 0 | 0 | 0 | 0 | 0 | 0 | 0.02 | 0 | 0 | 0 |
| ada9 | 0 | 0 | 0 | 0 | 0 | 0 | 0.02 | 0 | 0 | 0 |
| ada10 | 0 | 0 | 0 | 0 | 0 | 0 | 0.02 | 0 | 0 | 0 |
| ada11 | 0 | 0 | 0 | 0 | 0 | 0 | 0.02 | 0 | 0 | 0 |
| ada12 | 0 | 0 | 0 | 0 | 0 | 0 | 0.02 | 0 | 0 | 0 |
| ada13 | 0 | 0 | 0 | 0 | 0 | 0 | 0.02 | 0 | 0 | 0 |
| ada14 | 0 | 0 | 0 | 0 | 0 | 0 | 0.02 | 0 | 0 | 0 |
| ada15 | 0 | 0 | 0 | 0 | 0 | 0 | 0.02 | 0 | 0 | 0 |
| ada16 | 0 | 0 | 0 | 0 | 0 | 0 | 0.02 | 0 | 0 | 0 |
| ada17 | 0 | 0 | 0 | 0 | 0 | 0 | 0.02 | 0 | 0 | 0 |
| ada18 | 0 | 0 | 0 | 0 | 0 | 0 | 0.02 | 0 | 0 | 0 |
| ada19 | 0 | 0 | 0 | 0 | 0 | 0 | 0.02 | 0 | 0 | 0 |
| ada20 | 0 | 0 | 0 | 0 | 0 | 0 | 0.02 | 0 | 0 | 0 |
| ada21 | 0 | 0 | 0 | 0 | 0 | 0 | 0.02 | 0 | 0 | 0 |
| ada22 | 0 | 0 | 0 | 0 | 0 | 0 | 0.02 | 0 | 0 | 0 |
| ada23 | 0 | 0 | 0 | 0 | 0 | 0 | 0.02 | 0 | 0 | 0 |
| ada24 | 0 | 0 | 0 | 0 | 0 | 0 | 0.02 | 0 | 0 | 0 |
| ada25 | 0 | 0 | 0 | 0 | 0 | 0 | 0.02 | 0 | 0 | 0 |
| ada26 | 0 | 0 | 0 | 0 | 0 | 0 | 0.02 | 0 | 0 | 0 |
| ada27 | 0 | 0 | 0 | 0 | 0 | 0 | 0.02 | 0 | 0 | 0 |

| Gene | SMIM15 | SMIM3 | SMPD1 | SMU1 | SNHG5 | SNRPN | SPC25 | SPOCD1 | SPRY1 | SPSB4 |
|---|---|---|---|---|---|---|---|---|---|---|
| rf0 | 5.05E−19 | 0 | 0.005914 | 0.002194 | 0 | 0 | 0 | 0 | 0 | 0.006732 |
| rf1 | 0 | 0 | 0.000309 | 0 | 0.003431 | 0.00071 | 0.002454 | 0.000495 | 0.010405 | 0.007513 |
| rf2 | 0.000766 | 0.000652 | 0.002673 | 0 | 0 | 0 | 0.008696 | 0 | 0 | 0.00877 |
| rf3 | 9.70E−18 | 0.000671 | 0.004926 | 0.002569 | 0.003425 | 0.000902 | 0.000431 | 0 | 0.007665 | 0.011746 |
| rf4 | 0 | 0.001072 | 0.00678 | 0 | 0 | 0 | 0.00159 | 0 | 0 | 0.008072 |
| rf5 | 0.001212 | 0 | 0 | 0.003972 | 0.004239 | 0 | 0 | 0.001425 | 0.00214 | 0.006278 |
| rf6 | 0.000285 | 0.003128 | 0.003694 | 0.010581 | 0.003345 | 0.000365 | 0.006747 | 0 | 0.001046 | 0.007389 |
| rf7 | 0.000753 | 0.000643 | 0.003756 | 0 | 0.001016 | 0 | 0.000349 | 0 | 0.006087 | 0.010986 |
| rf8 | 0.001076 | 0 | 0.000348 | 0.000687 | 1.52E−18 | 0.000385 | 0.002786 | 0.000411 | 0.000464 | 0.007629 |
| rf9 | 0.00215 | 0.002514 | 0.003698 | 0.001998 | 0.008859 | 0 | 0.005557 | 0.000612 | 0.003759 | 0.007714 |
| rf10 | 0.004715 | 0.000383 | 0.007479 | 4.97E−18 | 0.006069 | 0.000335 | 0.001671 | 0.001538 | 0.002516 | 0.000829 |
| rf11 | 0.001164 | 0 | 0.004923 | 0.002713 | 0.003532 | 1.64E−18 | 0.00336 | 0.007625 | 0.000955 | 0.005283 |
| rf12 | 0.003705 | 0.00076 | 0.001237 | 0 | 0.006213 | 0.000586 | 0 | 0.002243 | 0.001494 | 0.001349 |
| rf13 | 0 | 0 | 0.005504 | 0.002813 | 0.007294 | 0.000885 | 0.001011 | 0.006061 | 0.004517 | 0.00318 |
| rf14 | 5.05E−19 | 0 | 0.005914 | 0.002194 | 0 | 0 | 0 | 0 | 0 | 0.006732 |
| rf15 | 0 | 0 | 0.000309 | 0 | 0.003431 | 0.00071 | 0.002454 | 0.000495 | 0.010405 | 0.007513 |
| rf16 | 0.000766 | 0.000652 | 0.002673 | 0 | 0 | 0 | 0.008696 | 0 | 0 | 0.00877 |
| rf17 | 9.70E−18 | 0.000671 | 0.004926 | 0.002569 | 0.003425 | 0.000902 | 0.000431 | 0 | 0.007665 | 0.011746 |
| rf18 | 0 | 0.001072 | 0.00678 | 0 | 0 | 0 | 0.00159 | 0 | 0 | 0.008072 |
| rf19 | 0.001212 | 0 | 0 | 0.003972 | 0.004239 | 0 | 0 | 0.001425 | 0.00214 | 0.006278 |
| rf20 | 0.000285 | 0.003128 | 0.003694 | 0.010581 | 0.003345 | 0.000365 | 0.006747 | 0 | 0.001046 | 0.007389 |
| rf21 | 0.000753 | 0.000643 | 0.003756 | 0 | 0.001016 | 0 | 0.000349 | 0 | 0.006087 | 0.010986 |
| rf22 | 0.001076 | 0 | 0.000348 | 0.000687 | 1.52E−18 | 0.000385 | 0.002786 | 0.000411 | 0.000464 | 0.007629 |
| rf23 | 0.00215 | 0.002514 | 0.003698 | 0.001998 | 0.008859 | 0 | 0.005557 | 0.000612 | 0.003759 | 0.007714 |
| rf24 | 0.004715 | 0.000383 | 0.007479 | 4.97E−18 | 0.006069 | 0.000335 | 0.001671 | 0.001538 | 0.002516 | 0.000829 |
| rf25 | 0.001164 | 0 | 0.004923 | 0.002713 | 0.003532 | 1.64E−18 | 0.00336 | 0.007625 | 0.000955 | 0.005283 |
| rf26 | 0.003705 | 0.00076 | 0.001237 | 0 | 0.006213 | 0.000586 | 0 | 0.002243 | 0.001494 | 0.001349 |
| rf27 | 0 | 0 | 0.005504 | 0.002813 | 0.007294 | 0.000885 | 0.001011 | 0.006061 | 0.004517 | 0.00318 |
| extraTrees0 | 0.003101 | 0.000753 | 0.002213 | 0.003848 | 0.002365 | 0.001006 | 0.002924 | 0.003067 | 0.003332 | 0.006163 |
| extraTrees1 | 0.001996 | 7.01E−19 | 0.001022 | 0.001363 | 0.000564 | 0.000451 | 0.008671 | 4.29E−05 | 0.003116 | 0.002262 |
| extraTrees2 | 1.26E−18 | 0 | 0.001654 | 0.001315 | 0.003118 | 0.002076 | 0.001571 | 0.001743 | 0.003865 | 0.000805 |
| extraTrees3 | 0.001837 | 0.001639 | 0.001275 | 0.001263 | 0.004096 | 0 | 0.002326 | 0.000638 | 0.002882 | 0.001411 |
| extraTrees4 | 0.00371 | 0.001393 | 0.001715 | 0.002695 | 0.001562 | 0.000924 | 0.001664 | 0.001433 | 0.004509 | 0.007954 |
| extraTrees5 | 0.000488 | 0.001043 | 0.001753 | 0.001759 | 0.003812 | 0.000384 | 0.007494 | 0.002369 | 0.00401 | 0.010486 |
| extraTrees6 | 0.000507 | 0.000432 | 5.26E−19 | 0.001091 | 0.003313 | 0.000886 | 0.004779 | 0.000796 | 0.005104 | 0.006095 |
| extraTrees7 | 0.00015 | 0.001736 | 0.000826 | 0.001545 | 0.001071 | 0.001475 | 0.001951 | 0.001206 | 0.003764 | 0.001566 |
| extraTrees8 | 0.000238 | 0.000706 | 0.001425 | 0.000285 | 0.003934 | 6.68E−18 | 0.003128 | 0.002493 | 0.005323 | 0.006114 |
| extraTrees9 | 0.003587 | 0.001145 | 0.000803 | 0.000245 | 0.002097 | 0.000492 | 0.002064 | 0.002592 | 0.003046 | 0.005795 |
| extraTrees10 | 0 | 2.90E−18 | 0.001206 | 0.002422 | 0.004063 | 0.003049 | 0.001885 | 0.000419 | 0.002091 | 0.008411 |
| extraTrees11 | 0.00058 | 0 | 0.001842 | 0.001125 | 0.004549 | 0.002422 | 0.000994 | 0.001739 | 0.000268 | 0.003378 |
| extraTrees12 | 0.001004 | 0.000268 | 0.00107 | 0.000333 | 0.002438 | 0.00173 | 0.005891 | 0.000271 | 0.000962 | 0.002562 |
| extraTrees13 | 0 | 0.002697 | 0.004056 | 0.002141 | 0.004303 | 5.26E−19 | 0.003385 | 0.001948 | 0.006368 | 0.003557 |
| extraTrees14 | 0.003101 | 0.000753 | 0.002213 | 0.003848 | 0.002365 | 0.001006 | 0.002924 | 0.003067 | 0.003332 | 0.006163 |
| extraTrees15 | 0.001996 | 7.01E−19 | 0.001022 | 0.001363 | 0.000564 | 0.000451 | 0.008671 | 4.29E−05 | 0.003116 | 0.002262 |
| extraTrees16 | 1.26E−18 | 0 | 0.001654 | 0.001315 | 0.003118 | 0.002076 | 0.001571 | 0.001743 | 0.003865 | 0.000805 |
| extraTrees17 | 0.001837 | 0.001639 | 0.001275 | 0.001263 | 0.004096 | 0 | 0.002326 | 0.000638 | 0.002882 | 0.001411 |
| extraTrees18 | 0.00371 | 0.001393 | 0.001715 | 0.002695 | 0.001562 | 0.000924 | 0.001664 | 0.001433 | 0.004509 | 0.007954 |
| extraTrees19 | 0.000488 | 0.001043 | 0.001753 | 0.001759 | 0.003812 | 0.000384 | 0.007494 | 0.002369 | 0.00401 | 0.010486 |
| extraTrees20 | 0.000507 | 0.000432 | 5.26E−19 | 0.001091 | 0.003313 | 0.000886 | 0.004779 | 0.000796 | 0.005104 | 0.006095 |
| extraTrees21 | 0.00015 | 0.001736 | 0.000826 | 0.001545 | 0.001071 | 0.001475 | 0.001951 | 0.001206 | 0.003764 | 0.001566 |
| extraTrees22 | 0.000238 | 0.000706 | 0.001425 | 0.000285 | 0.003934 | 6.68E−18 | 0.003128 | 0.002493 | 0.005323 | 0.006114 |
| extraTrees23 | 0.003587 | 0.001145 | 0.000803 | 0.000245 | 0.002097 | 0.000492 | 0.002064 | 0.002592 | 0.003046 | 0.005795 |
| extraTrees24 | 0 | 2.90E−18 | 0.001206 | 0.002422 | 0.004063 | 0.003049 | 0.001885 | 0.000419 | 0.002091 | 0.008411 |
| extraTrees25 | 0.00058 | 0 | 0.001842 | 0.001125 | 0.004549 | 0.002422 | 0.000994 | 0.001739 | 0.000268 | 0.003378 |
| extraTrees26 | 0.001004 | 0.000268 | 0.00107 | 0.000333 | 0.002438 | 0.00173 | 0.005891 | 0.000271 | 0.000962 | 0.002562 |
| extraTrees27 | 0 | 0.002697 | 0.004056 | 0.002141 | 0.004303 | 5.26E−19 | 0.003385 | 0.001948 | 0.006368 | 0.003557 |
| gbc0 | 0.018608 | 0 | 7.23E−05 | 0.01745 | 0.005214 | 0 | 0 | 0.001272 | 0.013514 | 0 |
| gbc1 | 0.010865 | 0 | 0.00151 | 0.001212 | 0.007157 | 0.000632 | 0 | 0.003707 | 0.052659 | 1.58E−05 |
| gbc2 | 0.011357 | 7.21E−05 | 0.000116 | 0 | 0 | 0.000491 | 0.001315 | 0.000147 | 0.001316 | 3.83E−05 |
| gbc3 | 3.67E−05 | 0 | 0 | 0 | 0.000788 | 0 | 0.002084 | 0.028515 | 0.045666 | 0.001349 |
| gbc4 | 0.024549 | 0.001779 | 0 | 0 | 0.000105 | 0.000743 | 0.00233 | 0.001961 | 5.19E−05 | 1.27E−06 |
| gbc5 | 0.011088 | 0 | 0.027291 | 0.000277 | 0.012221 | 0 | 0 | 7.14E−05 | 0 | 0.000576 |
| gbc6 | 0.005403 | 0 | 5.85E−06 | 0 | 0.010055 | 0 | 0.000184 | 0.000146 | 0 | 5.50E−05 |
| gbc7 | 0.00065 | 0 | 0 | 3.93E−07 | 0 | 0 | 0 | 0.016754 | 0.000111 | 0.000216 |
| gbc8 | 0.001199 | 0 | 1.17E−05 | 0.000406 | 0 | 0 | 0 | 0 | 0.000621 | 4.24E−06 |
| gbc9 | 0.001273 | 0.000747 | 0.005058 | 0.023324 | 0 | 0 | 0 | 0.001096 | 0.003633 | 0 |
| gbc10 | 0 | 0 | 6.54E−06 | 0 | 0 | 0 | 0 | 8.62E−05 | 0.001705 | 0 |
| gbc11 | 0.000443 | 0 | 0 | 1.28E−06 | 0.000243 | 0 | 0.001144 | 0.000324 | 0.003159 | 0 |
| gbc12 | 0 | 0 | 0 | 0 | 0 | 6.41E−05 | 0 | 2.60E−07 | 0.000101 | 0.002277 |
| gbc13 | 0.001343 | 0.005007 | 0.000721 | 0 | 0.001548 | 0 | 0 | 0 | 0.003379 | 0.001678 |
| gbc14 | 0.018608 | 0 | 7.23E−05 | 0.01745 | 0.005214 | 0 | 0 | 0.001272 | 0.013514 | 0 |
| gbc15 | 0.010865 | 0 | 0.00151 | 0.001212 | 0.007157 | 0.000632 | 0 | 0.003707 | 0.052659 | 1.58E−05 |
| gbc16 | 0.011357 | 7.21E−05 | 0.000116 | 0 | 0 | 0.000491 | 0.001315 | 0.000147 | 0.001316 | 3.83E−05 |
| gbc17 | 3.67E−05 | 0 | 0 | 0 | 0.000788 | 0 | 0.002084 | 0.028515 | 0.045666 | 0.001349 |
| gbc18 | 0.024549 | 0.001779 | 0 | 0 | 0.000105 | 0.000743 | 0.00233 | 0.001961 | 5.19E−05 | 1.27E−06 |
| gbc19 | 0.011088 | 0 | 0.027291 | 0.000277 | 0.012221 | 0 | 0 | 7.14E−05 | 0 | 0.000576 |
| gbc20 | 0.005403 | 0 | 5.85E−06 | 0 | 0.010055 | 0 | 0.000184 | 0.000146 | 0 | 5.50E−05 |
| gbc21 | 0.00065 | 0 | 0 | 3.93E−07 | 0 | 0 | 0 | 0.016754 | 0.000111 | 0.000216 |
| gbc22 | 0.001199 | 0 | 1.17E−05 | 0.000406 | 0 | 0 | 0 | 0 | 0.000621 | 4.24E−06 |
| gbc23 | 0.001273 | 0.000747 | 0.005058 | 0.023324 | 0 | 0 | 0 | 0.001096 | 0.003633 | 0 |
| gbc24 | 0 | 0 | 6.54E−06 | 0 | 0 | 0 | 0 | 8.62E−05 | 0.001705 | 0 |
| gbc25 | 0.000443 | 0 | 0 | 1.28E−06 | 0.000243 | 0 | 0.001144 | 0.000324 | 0.003159 | 0 |
| gbc26 | 0 | 0 | 0 | 0 | 0 | 6.41E−05 | 0 | 2.60E−07 | 0.000101 | 0.002277 |
| gbc27 | 0.001343 | 0.005007 | 0.000721 | 0 | 0.001548 | 0 | 0 | 0 | 0.003379 | 0.001678 |
| ada0 | 0 | 0 | 0 | 0 | 0 | 0 | 0 | 0 | 0.02 | 0 |
| ada1 | 0 | 0 | 0 | 0 | 0 | 0 | 0 | 0 | 0.02 | 0 |
| ada2 | 0 | 0 | 0 | 0 | 0 | 0 | 0 | 0 | 0.02 | 0 |
| ada3 | 0 | 0 | 0 | 0 | 0 | 0 | 0 | 0 | 0.02 | 0 |
| ada4 | 0 | 0 | 0 | 0 | 0 | 0 | 0 | 0 | 0.02 | 0 |
| ada5 | 0 | 0 | 0 | 0 | 0 | 0 | 0 | 0 | 0.02 | 0 |
| ada6 | 0 | 0 | 0 | 0 | 0 | 0 | 0 | 0 | 0.02 | 0 |
| ada7 | 0 | 0 | 0 | 0 | 0 | 0 | 0 | 0 | 0.02 | 0 |
| ada8 | 0 | 0 | 0 | 0 | 0 | 0 | 0 | 0 | 0.02 | 0 |
| ada9 | 0 | 0 | 0 | 0 | 0 | 0 | 0 | 0 | 0.02 | 0 |
| ada10 | 0 | 0 | 0 | 0 | 0 | 0 | 0 | 0 | 0.02 | 0 |
| ada11 | 0 | 0 | 0 | 0 | 0 | 0 | 0 | 0 | 0.02 | 0 |
| ada12 | 0 | 0 | 0 | 0 | 0 | 0 | 0 | 0 | 0.02 | 0 |
| ada13 | 0 | 0 | 0 | 0 | 0 | 0 | 0 | 0 | 0.02 | 0 |
| ada14 | 0 | 0 | 0 | 0 | 0 | 0 | 0 | 0 | 0.02 | 0 |
| ada15 | 0 | 0 | 0 | 0 | 0 | 0 | 0 | 0 | 0.02 | 0 |
| ada16 | 0 | 0 | 0 | 0 | 0 | 0 | 0 | 0 | 0.02 | 0 |
| ada17 | 0 | 0 | 0 | 0 | 0 | 0 | 0 | 0 | 0.02 | 0 |
| ada18 | 0 | 0 | 0 | 0 | 0 | 0 | 0 | 0 | 0.02 | 0 |
| ada19 | 0 | 0 | 0 | 0 | 0 | 0 | 0 | 0 | 0.02 | 0 |
| ada20 | 0 | 0 | 0 | 0 | 0 | 0 | 0 | 0 | 0.02 | 0 |
| ada21 | 0 | 0 | 0 | 0 | 0 | 0 | 0 | 0 | 0.02 | 0 |
| ada22 | 0 | 0 | 0 | 0 | 0 | 0 | 0 | 0 | 0.02 | 0 |
| ada23 | 0 | 0 | 0 | 0 | 0 | 0 | 0 | 0 | 0.02 | 0 |
| ada24 | 0 | 0 | 0 | 0 | 0 | 0 | 0 | 0 | 0.02 | 0 |
| ada25 | 0 | 0 | 0 | 0 | 0 | 0 | 0 | 0 | 0.02 | 0 |
| ada26 | 0 | 0 | 0 | 0 | 0 | 0 | 0 | 0 | 0.02 | 0 |
| ada27 | 0 | 0 | 0 | 0 | 0 | 0 | 0 | 0 | 0.02 | 0 |
| Gene | ST3GAL1 | STAM2 | STIM1 | STIMATE | STK40 | SUGT1 | TADA1 | TAF12 | TANK | TAX1BP1 |
| rf0 | 0.002162 | 0.000665 | 0.00097 | 0.002738 | 1.07E−18 | 0 | 0.000477 | 0 | 0.001064 | 0.002026 |
| rf1 | 0.001634 | 0 | 0.002004 | 0.001366 | 0 | 0.000937 | 3.36E−19 | 0.005056 | 0.015565 | 0.005446 |
| rf2 | 0.000358 | 0 | 0.000703 | 0.000374 | 0 | 0.007431 | 0.000984 | 0 | 0.004009 | 0.010667 |
| rf3 | 0.00258 | 0 | 0 | 0 | 0.001706 | 0 | 0 | 0 | 0.002257 | 0.00245 |
| rf4 | 2.76E−05 | 0.003985 | 0.000341 | 0 | 0.005134 | 0.003061 | 0.000634 | 0 | 0 | 0.006925 |
| rf5 | 0 | 1.10E−18 | 0.000764 | 0 | 0.004607 | 0.001223 | 7.81E−19 | 0 | 0.009712 | 0 |
| rf6 | 0.002138 | 0.004237 | 0.00113 | 0.000624 | 0 | 0.001245 | 0 | 2.07E−18 | 0.002966 | 0.003101 |
| rf7 | 0.001416 | 0.007513 | 1.60E−18 | 0.00186 | 2.27E−18 | 0 | 0 | 0.00073 | 0.006416 | 0.001062 |
| rf8 | 0 | 0 | 0 | 0.000685 | 0 | 0.001828 | 2.08E−18 | 8.71E−19 | 0.004083 | 0.002938 |
| rf9 | 0.002282 | 1.10E−18 | 0.00035 | 0.00184 | 0.002531 | 0.002032 | 0.001945 | 0.000818 | 0.002835 | 0.005442 |
| rf10 | 0.00357 | 0.000315 | 0.005916 | 0.001007 | 0 | 0 | 0.000481 | 4.57E−19 | 0.010914 | 0.003688 |
| rf11 | 1.52E−18 | 0.001774 | 1.69E−17 | 0.00289 | 0.005275 | 1.60E−18 | 0.001278 | 1.05E−17 | 0.001789 | 0 |
| rf12 | 0.002086 | 0.004315 | 0 | 0.001509 | 0 | 0.000954 | 0.001209 | 0 | 0.011564 | 0.001868 |
| rf13 | 0.001899 | 0.003763 | 7.26E−19 | 0.001031 | 0 | 0 | 0.000574 | 0.001865 | 0 | 0 |
| rf14 | 0.002162 | 0.000665 | 0.00097 | 0.002738 | 1.07E−18 | 0 | 0.000477 | 0 | 0.001064 | 0.002026 |
| rf15 | 0.001634 | 0 | 0.002004 | 0.001366 | 0 | 0.000937 | 3.36E−19 | 0.005056 | 0.015565 | 0.005446 |
| rf16 | 0.000358 | 0 | 0.000703 | 0.000374 | 0 | 0.007431 | 0.000984 | 0 | 0.004009 | 0.010667 |
| rf17 | 0.00258 | 0 | 0 | 0 | 0.001706 | 0 | 0 | 0 | 0.002257 | 0.00245 |
| rf18 | 2.76E−05 | 0.003985 | 0.000341 | 0 | 0.005134 | 0.003061 | 0.000634 | 0 | 0 | 0.006925 |
| rf19 | 0 | 1.10E−18 | 0.000764 | 0 | 0.004607 | 0.001223 | 7.81E−19 | 0 | 0.009712 | 0 |
| rf20 | 0.002138 | 0.004237 | 0.00113 | 0.000624 | 0 | 0.001245 | 0 | 2.07E−18 | 0.002966 | 0.003101 |
| rf21 | 0.001416 | 0.007513 | 1.60E−18 | 0.00186 | 2.27E−18 | 0 | 0 | 0.00073 | 0.006416 | 0.001062 |
| rf22 | 0 | 0 | 0 | 0.000685 | 0 | 0.001828 | 2.08E−18 | 8.71E−19 | 0.004083 | 0.002938 |
| rf23 | 0.002282 | 1.10E−18 | 0.00035 | 0.00184 | 0.002531 | 0.002032 | 0.001945 | 0.000818 | 0.002835 | 0.005442 |
| rf24 | 0.00357 | 0.000315 | 0.005916 | 0.001007 | 0 | 0 | 0.000481 | 4.57E−19 | 0.010914 | 0.003688 |
| rf25 | 1.52E−18 | 0.001774 | 1.69E−17 | 0.00289 | 0.005275 | 1.60E−18 | 0.001278 | 1.05E−17 | 0.001789 | 0 |
| rf26 | 0.002086 | 0.004315 | 0 | 0.001509 | 0 | 0.000954 | 0.001209 | 0 | 0.011564 | 0.001868 |
| rf27 | 0.001899 | 0.003763 | 7.26E−19 | 0.001031 | 0 | 0 | 0.000574 | 0.001865 | 0 | 0 |
| extraTrees0 | 0.002665 | 0.000525 | 0.000876 | 0.002401 | 0.002081 | 0 | 0.00134 | 0.002063 | 0.003455 | 0.003029 |
| extraTrees1 | 0.000613 | 0.001161 | 0.004279 | 0.003758 | 0.00595 | 0.00137 | 0.000491 | 0.001488 | 0.000508 | 0.003355 |
| extraTrees2 | 0.002154 | 0.001659 | 5.26E−05 | 0.002059 | 0.001676 | 0.00067 | 0.000388 | 0.000963 | 0.006403 | 0.004163 |
| extraTrees3 | 0.001238 | 0.000778 | 0.000758 | 0.003029 | 0.000762 | 0 | 0.000827 | 0.001161 | 0.003963 | 0.001766 |
| extraTrees4 | 0.000316 | 0.002019 | 0.002373 | 0.002486 | 0.001934 | 0.000688 | 0.000563 | 0.00104 | 0.009614 | 0.001806 |
| extraTrees5 | 0.001771 | 0.000681 | 0.000393 | 0.000738 | 0.000596 | 0.001572 | 0.000489 | 0.001275 | 0.004788 | 0.000903 |
| extraTrees6 | 0.001242 | 0.002916 | 0.001121 | 0.002428 | 0.00033 | 0.000665 | 0.000271 | 0.000238 | 0.004507 | 0.001087 |
| extraTrees7 | 0.001712 | 0.001389 | 0.006499 | 0.001324 | 0.003512 | 0.000637 | 6.16E−05 | 0.00034 | 0.00598 | 0.001987 |
| extraTrees8 | 0.00116 | 0.001812 | 0.000669 | 0.001198 | 0.004864 | 0.000888 | 0.002768 | 0.002222 | 0.001124 | 0.00275 |
| extraTrees9 | 0.001031 | 0.000668 | 0.002017 | 0.001227 | 0.002949 | 0.001025 | 0.000525 | 0.001123 | 0.002523 | 0.001147 |
| extraTrees10 | 0.000963 | 0.001251 | 0.005508 | 0 | 0.001199 | 0.002647 | 0.001466 | 0.000538 | 0.001701 | 0.000475 |
| extraTrees11 | 0.001202 | 0.001061 | 0.002437 | 0.000657 | 0.000798 | 0.001386 | 1.90E−18 | 0.001493 | 0.002528 | 0.001736 |
| extraTrees12 | 0.000531 | 0.00025 | 0.001071 | 0.002354 | 0.001832 | 0.000867 | 0.000892 | 0.001727 | 0.002907 | 0.001886 |
| extraTrees13 | 0.000253 | 0.001605 | 0.000496 | 0.000627 | 0.002211 | 0.000244 | 0.002468 | 0.00178 | 0.002089 | 0.003772 |
| extraTrees14 | 0.002665 | 0.000525 | 0.000876 | 0.002401 | 0.002081 | 0 | 0.00134 | 0.002063 | 0.003455 | 0.003029 |
| extraTrees15 | 0.000613 | 0.001161 | 0.004279 | 0.003758 | 0.00595 | 0.00137 | 0.000491 | 0.001488 | 0.000508 | 0.003355 |
| extraTrees16 | 0.002154 | 0.001659 | 5.26E−05 | 0.002059 | 0.001676 | 0.00067 | 0.000388 | 0.000963 | 0.006403 | 0.004163 |
| extraTrees17 | 0.001238 | 0.000778 | 0.000758 | 0.003029 | 0.000762 | 0 | 0.000827 | 0.001161 | 0.003963 | 0.001766 |
| extraTrees18 | 0.000316 | 0.002019 | 0.002373 | 0.002486 | 0.001934 | 0.000688 | 0.000563 | 0.00104 | 0.009614 | 0.001806 |
| extraTrees19 | 0.001771 | 0.000681 | 0.000393 | 0.000738 | 0.000596 | 0.001572 | 0.000489 | 0.001275 | 0.004788 | 0.000903 |
| extraTrees20 | 0.001242 | 0.002916 | 0.001121 | 0.002428 | 0.00033 | 0.000665 | 0.000271 | 0.000238 | 0.004507 | 0.001087 |
| extraTrees21 | 0.001712 | 0.001389 | 0.006499 | 0.001324 | 0.003512 | 0.000637 | 6.16E−05 | 0.00034 | 0.00598 | 0.001987 |
| extraTrees22 | 0.00116 | 0.001812 | 0.000669 | 0.001198 | 0.004864 | 0.000888 | 0.002768 | 0.002222 | 0.001124 | 0.00275 |
| extraTrees23 | 0.001031 | 0.000668 | 0.002017 | 0.001227 | 0.002949 | 0.001025 | 0.000525 | 0.001123 | 0.002523 | 0.001147 |
| extraTrees24 | 0.000963 | 0.001251 | 0.005508 | 0 | 0.001199 | 0.002647 | 0.001466 | 0.000538 | 0.001701 | 0.000475 |
| extraTrees25 | 0.001202 | 0.001061 | 0.002437 | 0.000657 | 0.000798 | 0.001386 | 1.90E−18 | 0.001493 | 0.002528 | 0.001736 |
| extraTrees26 | 0.000531 | 0.00025 | 0.001071 | 0.002354 | 0.001832 | 0.000867 | 0.000892 | 0.001727 | 0.002907 | 0.001886 |
| extraTrees27 | 0.000253 | 0.001605 | 0.000496 | 0.000627 | 0.002211 | 0.000244 | 0.002468 | 0.00178 | 0.002089 | 0.003772 |
| gbc0 | 3.82E−05 | 0 | 0.000137 | 0 | 0 | 0 | 0 | 4.20E−05 | 0 | 0.013607 |
| gbc1 | 0 | 0 | 0 | 0 | 9.58E−06 | 0 | 0 | 0 | 0.002977 | 0.001329 |
| gbc2 | 0.000356 | 0 | 0 | 0.000309 | 0 | 0.004576 | 0 | 8.41E−05 | 3.63E−05 | 0 |
| gbc3 | 6.30E−06 | 0.000138 | 4.98E−05 | 0 | 0 | 0.000262 | 0 | 1.17E−06 | 0 | 0.008719 |
| gbc4 | 0.001058 | 0 | 0 | 0 | 0 | 0 | 0 | 0 | 0 | 0.000216 |
| gbc5 | 0.00597 | 0.001525 | 0.001394 | 0 | 0 | 2.84E−06 | 8.34E−05 | 0 | 6.42E−06 | 0.0004 |
| gbc6 | 0 | 0.012314 | 8.29E−07 | 0 | 0.050341 | 0.000974 | 0.001379 | 0 | 0.029361 | 0.000874 |
| gbc7 | 0 | 0.002078 | 0 | 0.010133 | 2.66E−05 | 0 | 0.00013 | 0 | 0 | 2.78E−05 |
| gbc8 | 0 | 0.023357 | 0 | 5.15E−07 | 0 | 0 | 0 | 0.000168 | 9.75E−05 | 0.008647 |
| gbc9 | 0.002731 | 0 | 7.91E−05 | 0 | 0 | 9.71E−05 | 0.011668 | 1.64E−05 | 0.000496 | 0.000412 |
| gbc10 | 0.003653 | 0 | 0 | 0.001733 | 0 | 0 | 0 | 0 | 0.000182 | 8.77E−05 |
| gbc11 | 0.001852 | 0.002661 | 0 | 0 | 0 | 0 | 0 | 0 | 3.94E−05 | 0.00399 |
| gbc12 | 0 | 0.001343 | 0.001633 | 0 | 0.01902 | 4.79E−06 | 0 | 0 | 0 | 0.003729 |
| gbc13 | 0 | 0 | 7.63E−06 | 0.001596 | 0 | 5.10E−06 | 0.000376 | 0.003286 | 0 | 0.001005 |
| gbc14 | 3.82E−05 | 0 | 0.000137 | 0 | 0 | 0 | 0 | 4.20E−05 | 0 | 0.013607 |
| gbc15 | 0 | 0 | 0 | 0 | 9.58E−06 | 0 | 0 | 0 | 0.002977 | 0.001329 |
| gbc16 | 0.000356 | 0 | 0 | 0.000309 | 0 | 0.004576 | 0 | 8.41E−05 | 3.63E−05 | 0 |
| gbc17 | 6.30E−06 | 0.000138 | 4.98E−05 | 0 | 0 | 0.000262 | 0 | 1.17E−06 | 0 | 0.008719 |
| gbc18 | 0.001058 | 0 | 0 | 0 | 0 | 0 | 0 | 0 | 0 | 0.000216 |
| gbc19 | 0.00597 | 0.001525 | 0.001394 | 0 | 0 | 2.84E−06 | 8.34E−05 | 0 | 6.42E−06 | 0.0004 |
| gbc20 | 0 | 0.012314 | 8.29E−07 | 0 | 0.050341 | 0.000974 | 0.001379 | 0 | 0.029361 | 0.000874 |
| gbc21 | 0 | 0.002078 | 0 | 0.010133 | 2.66E−05 | 0 | 0.00013 | 0 | 0 | 2.78E−05 |
| gbc22 | 0 | 0.023357 | 0 | 5.15E−07 | 0 | 0 | 0 | 0.000168 | 9.75E−05 | 0.008647 |
| gbc23 | 0.002731 | 0 | 7.91E−05 | 0 | 0 | 9.71E−05 | 0.011668 | 1.64E−05 | 0.000496 | 0.000412 |
| gbc24 | 0.003653 | 0 | 0 | 0.001733 | 0 | 0 | 0 | 0 | 0.000182 | 8.77E−05 |
| gbc25 | 0.001852 | 0.002661 | 0 | 0 | 0 | 0 | 0 | 0 | 3.94E−05 | 0.00399 |
| gbc26 | 0 | 0.001343 | 0.001633 | 0 | 0.01902 | 4.79E−06 | 0 | 0 | 0 | 0.003729 |
| gbc27 | 0 | 0 | 7.63E−06 | 0.001596 | 0 | 5.10E−06 | 0.000376 | 0.003286 | 0 | 0.001005 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | TAX1BP3 | TBC1D13 | TBC1D20 | TBC1D22A | TCEANC | TEX30 | TFAM | TGDS | TIPRL | TMCO3 |
|---|---|---|---|---|---|---|---|---|---|---|
| rf0 | 0.001132 | 0.000288 | 0 | 2.93E−18 | 0.002563 | 0 | 0 | 8.32E−18 | 0.001473 | 0.00482 |
| rf1 | 6.86E−19 | 0 | 0 | 0 | 0.000797 | 0 | 0 | 0.007701 | 0.000999 | 0.000959 |
| rf2 | 0 | 4.57E−19 | 0 | 0.00035 | 0.002371 | 0.001079 | 0 | 0.005866 | 0.003563 | 0 |
| rf3 | 0 | 0.003354 | 0.002355 | 0.001145 | 0 | 0.000281 | 0.002844 | 0.007039 | 0 | 0.001318 |
| rf4 | 0 | 0 | 0.011026 | 0.000899 | 0.010061 | 0.000288 | 0.001303 | 0.004342 | 0 | 1.26E−18 |
| rf5 | 0.000319 | 0.00035 | 0.001195 | 0 | 0.003171 | 0 | 0 | 0.005083 | 0 | 0.011142 |
| rf6 | 0 | 0.001059 | 0 | 0.001205 | 7.26E−19 | 0.000285 | 0.000589 | 0.000823 | 0.003364 | 0.004715 |
| rf7 | 0.000744 | 0.00223 | 0.001883 | 0.00115 | 0.001731 | 0.000335 | 0 | 0.0099 | 0.000588 | 0.000687 |
| rf8 | 0.000366 | 0 | 0.000738 | 0.00516 | 0.004139 | 0 | 2.94E−18 | 0.011674 | 0 | 0.002672 |
| rf9 | 0.003312 | 8.49E−19 | 0.008631 | 0.000982 | 0.000319 | 0.00066 | 0 | 0.00196 | 0 | 0.005666 |
| rf10 | 0.003622 | 0.000353 | 0.001566 | 0 | 0 | 0.000358 | 9.90E−18 | 0.01189 | 0 | 0.002205 |
| rf11 | 2.18E−17 | 0 | 0 | 2.08E−18 | 0.015548 | 0.000547 | 0 | 0 | 0.004359 | 0 |
| rf12 | 0.002256 | 0.000594 | 0.004811 | 0 | 0 | 0.001143 | 0.003774 | 0.007644 | 0.005412 | 0.001275 |
| rf13 | 0 | 0.002146 | 0.008496 | 0.000631 | 0.002168 | 0.001241 | 0.002228 | 0.015113 | 0.002405 | 0.008168 |
| rf14 | 0.001132 | 0.000288 | 0 | 2.93E−18 | 0.002563 | 0 | 0 | 8.32E−18 | 0.001473 | 0.00482 |
| rf15 | 6.86E−19 | 0 | 0 | 0 | 0.000797 | 0 | 0 | 0.007701 | 0.000999 | 0.000959 |
| rf16 | 0 | 4.57E−19 | 0 | 0.00035 | 0.002371 | 0.001079 | 0 | 0.005866 | 0.003563 | 0 |
| rf17 | 0 | 0.003354 | 0.002355 | 0.001145 | 0 | 0.000281 | 0.002844 | 0.007039 | 0 | 0.001318 |
| rf18 | 0 | 0 | 0.011026 | 0.000899 | 0.010061 | 0.000288 | 0.001303 | 0.004342 | 0 | 1.26E−18 |
| rf19 | 0.000319 | 0.00035 | 0.001195 | 0 | 0.003171 | 0 | 0 | 0.005083 | 0 | 0.011142 |
| rf20 | 0 | 0.001059 | 0 | 0.001205 | 7.26E−19 | 0.000285 | 0.000589 | 0.000823 | 0.003364 | 0.004715 |
| rf21 | 0.000744 | 0.00223 | 0.001883 | 0.00115 | 0.001731 | 0.000335 | 0 | 0.0099 | 0.000588 | 0.000687 |
| rf22 | 0.000366 | 0 | 0.000738 | 0.00516 | 0.004139 | 0 | 2.94E−18 | 0.011674 | 0 | 0.002672 |
| rf23 | 0.003312 | 8.49E−19 | 0.008631 | 0.000982 | 0.000319 | 0.00066 | 0 | 0.00196 | 0 | 0.005666 |
| rf24 | 0.003622 | 0.000353 | 0.001566 | 0 | 0 | 0.000358 | 9.90E−18 | 0.01189 | 0 | 0.002205 |
| rf25 | 2.18E−17 | 0 | 0 | 2.08E−18 | 0.015548 | 0.000547 | 0 | 0 | 0.004359 | 0 |
| rf26 | 0.002256 | 0.000594 | 0.004811 | 0 | 0 | 0.001143 | 0.003774 | 0.007644 | 0.005412 | 0.001275 |
| rf27 | 0 | 0.002146 | 0.008496 | 0.000631 | 0.002168 | 0.001241 | 0.002228 | 0.015113 | 0.002405 | 0.008168 |
| extraTrees0 | 0.002413 | 0.000277 | 0.001054 | 0.001245 | 0 | 0.00032 | 0.000291 | 0.00224 | 0.000238 | 0.006328 |
| extraTrees1 | 0 | 0.002497 | 0.00188 | 0.002126 | 0.003955 | 1.26E−18 | 0.001043 | 0.000377 | 0.00118 | 0.003598 |
| extraTrees2 | 0.004501 | 0.002335 | 0.001028 | 0.000929 | 0.002571 | 0.000567 | 0.000777 | 0.00636 | 2.29E−18 | 0.005394 |
| extraTrees3 | 0.000723 | 0.00119 | 0.00212 | 0.000508 | 0.00352 | 0.000939 | 0.000235 | 0.003291 | 0.001098 | 0.003985 |
| extraTrees4 | 0.001849 | 0.000238 | 0.002046 | 2.53E−18 | 0.003499 | 0.00062 | 0.000817 | 0.004127 | 0 | 0.004148 |
| extraTrees5 | 0.001849 | 0.00113 | 0.003058 | 0.001307 | 0.001583 | 0 | 0.001045 | 0.00996 | 0.003409 | 0.001946 |
| extraTrees6 | 0.001257 | 0.000901 | 0.006599 | 0.001333 | 0.000828 | 0.000268 | 0.000928 | 0.00355 | 0.001915 | 0.005782 |
| extraTrees7 | 0.000641 | 0.000739 | 0.001547 | 0.000846 | 0.002808 | 0 | 4.10E−19 | 0.002407 | 0.00211 | 0.002817 |
| extraTrees8 | 0.001856 | 0.00047 | 0.002871 | 0.001746 | 0.001917 | 0.001321 | 0.000268 | 0.002254 | 0.001833 | 0.003709 |
| extraTrees9 | 0.002364 | 0.002229 | 0.000256 | 0.002255 | 0.001749 | 0.000238 | 0.000306 | 0.003738 | 0.000915 | 0.006296 |
| extraTrees10 | 0.002779 | 0.000642 | 0.003806 | 0.001601 | 0.001752 | 0.001016 | 0.000493 | 0.001573 | 0.000268 | 0.00404 |
| extraTrees11 | 0.001302 | 0.001277 | 0.001839 | 0.00028 | 0.001019 | 0.000957 | 0.000486 | 0.007437 | 0.002571 | 0.004419 |
| extraTrees12 | 0.001157 | 0.001928 | 0.00128 | 0.000784 | 0.002638 | 0 | 0.002692 | 0.004736 | 0.002081 | 0.004775 |
| extraTrees13 | 0.000194 | 0.001928 | 0.001275 | 0.001807 | 0.001988 | 0.001678 | 6.15E−19 | 0.007813 | 0.001817 | 0.003523 |
| extraTrees14 | 0.002413 | 0.000277 | 0.001054 | 0.001245 | 0 | 0.00032 | 0.000291 | 0.00224 | 0.000238 | 0.006328 |
| extraTrees15 | 0 | 0.002497 | 0.00188 | 0.002126 | 0.003955 | 1.26E−18 | 0.001043 | 0.000377 | 0.00118 | 0.003598 |
| extraTrees16 | 0.004501 | 0.002335 | 0.001028 | 0.000929 | 0.002571 | 0.000567 | 0.000777 | 0.00636 | 2.29E−18 | 0.005394 |
| extraTrees17 | 0.000723 | 0.00119 | 0.00212 | 0.000508 | 0.00352 | 0.000939 | 0.000235 | 0.003291 | 0.001098 | 0.003985 |
| extraTrees18 | 0.001849 | 0.000238 | 0.002046 | 2.53E−18 | 0.003499 | 0.00062 | 0.000817 | 0.004127 | 0 | 0.004148 |
| extraTrees19 | 0.001849 | 0.00113 | 0.003058 | 0.001307 | 0.001583 | 0 | 0.001045 | 0.00996 | 0.003409 | 0.001946 |
| extraTrees20 | 0.001257 | 0.000901 | 0.006599 | 0.001333 | 0.000828 | 0.000268 | 0.000928 | 0.00355 | 0.001915 | 0.005782 |
| extraTrees21 | 0.000641 | 0.000739 | 0.001547 | 0.000846 | 0.002808 | 0 | 4.10E−19 | 0.002407 | 0.00211 | 0.002817 |
| extraTrees22 | 0.001856 | 0.00047 | 0.002871 | 0.001746 | 0.001917 | 0.001321 | 0.000268 | 0.002254 | 0.001833 | 0.003709 |
| extraTrees23 | 0.002364 | 0.002229 | 0.000256 | 0.002255 | 0.001749 | 0.000238 | 0.000306 | 0.003738 | 0.000915 | 0.006296 |
| extraTrees24 | 0.002779 | 0.000642 | 0.003806 | 0.001601 | 0.001752 | 0.001016 | 0.000493 | 0.001573 | 0.000268 | 0.00404 |
| extraTrees25 | 0.001302 | 0.001277 | 0.001839 | 0.00028 | 0.001019 | 0.000957 | 0.000486 | 0.007437 | 0.002571 | 0.004419 |
| extraTrees26 | 0.001157 | 0.001928 | 0.00128 | 0.000784 | 0.002638 | 0 | 0.002692 | 0.004736 | 0.002081 | 0.004775 |
| extraTrees27 | 0.000194 | 0.001928 | 0.001275 | 0.001807 | 0.001988 | 0.001678 | 6.15E−19 | 0.007813 | 0.001817 | 0.003523 |
| gbc0 | 5.85E−05 | 4.01E−05 | 0.000157 | 0 | 0 | 0.000782 | 0 | 0.001426 | 2.43E−06 | 0.006773 |
| gbc1 | 0 | 0.000197 | 0.008653 | 0.001331 | 0 | 0 | 0 | 0 | 0.00231 | 0.000326 |
| gbc2 | 0 | 0 | 0.000174 | 0 | 0 | 0.000109 | 0.000132 | 0.001761 | 0.00349 | 0.034754 |
| gbc3 | 0.002598 | 0 | 4.69E−06 | 0.020153 | 0 | 5.74E−05 | 0 | 0.009768 | 2.98E−05 | 0.001919 |
| gbc4 | 0 | 0 | 0 | 0 | 0.005376 | 0.000126 | 0 | 0.003733 | 0 | 0.000704 |
| gbc5 | 0.000827 | 1.59E−05 | 0 | 3.42E−05 | 0 | 0 | 0 | 0.001148 | 0.000135 | 0.001457 |
| gbc6 | 0 | 0.000235 | 5.28E−05 | 0.008862 | 0 | 0 | 0 | 0 | 0 | 0.001379 |
| gbc7 | 0 | 0 | 0 | 0 | 0.007441 | 2.35E−05 | 0 | 0.000993 | 0.000818 | 1.81E−05 |
| gbc8 | 8.59E−05 | 0.001661 | 2.71E−05 | 7.74E−05 | 0.000237 | 0.00171 | 0 | 8.36E−05 | 0 | 2.29E−05 |
| gbc9 | 0 | 0 | 0 | 0.000372 | 0.001299 | 0 | 0 | 0.023538 | 0.000529 | 0.021567 |
| gbc10 | 0 | 0 | 0.029257 | 0 | 0 | 6.55E−05 | 0 | 0.003509 | 0.000126 | 0.000478 |
| gbc11 | 0 | 0 | 0 | 0 | 0.018316 | 0 | 0 | 0.000906 | 0.000167 | 0.016529 |
| gbc12 | 7.51E−05 | 0 | 6.75E−05 | 9.33E−05 | 0.00057 | 0.00018 | 0 | 0 | 0.001515 | 0.000315 |
| gbc13 | 0 | 0 | 0 | 0.000381 | 0 | 6.80E−05 | 0 | 0.015655 | 0.006555 | 0.000179 |
| gbc14 | 5.85E−05 | 4.01E−05 | 0.000157 | 0 | 0 | 0.000782 | 0 | 0.001426 | 2.43E−06 | 0.006773 |
| gbc15 | 0 | 0.000197 | 0.008653 | 0.001331 | 0 | 0 | 0 | 0 | 0.00231 | 0.000326 |
| gbc16 | 0 | 0 | 0.000174 | 0 | 0 | 0.000109 | 0.000132 | 0.001761 | 0.00349 | 0.034754 |
| gbc17 | 0.002598 | 0 | 4.69E−06 | 0.020153 | 0 | 5.74E−05 | 0 | 0.009768 | 2.98E−05 | 0.001919 |
| gbc18 | 0 | 0 | 0 | 0 | 0.005376 | 0.000126 | 0 | 0.003733 | 0 | 0.000704 |
| gbc19 | 0.000827 | 1.59E−05 | 0 | 3.42E−05 | 0 | 0 | 0 | 0.001148 | 0.000135 | 0.001457 |
| gbc20 | 0 | 0.000235 | 5.28E−05 | 0.008862 | 0 | 0 | 0 | 0 | 0 | 0.001379 |
| gbc21 | 0 | 0 | 0 | 0 | 0.007441 | 2.35E−05 | 0 | 0.000993 | 0.000818 | 1.81E−05 |
| gbc22 | 8.59E−05 | 0.001661 | 2.71E−05 | 7.74E−05 | 0.000237 | 0.00171 | 0 | 8.36E−05 | 0 | 2.29E−05 |
| gbc23 | 0 | 0 | 0 | 0.000372 | 0.001299 | 0 | 0 | 0.023538 | 0.000529 | 0.021567 |
| gbc24 | 0 | 0 | 0.029257 | 0 | 0 | 6.55E−05 | 0 | 0.003509 | 0.000126 | 0.000478 |
| gbc25 | 0 | 0 | 0 | 0 | 0.018316 | 0 | 0 | 0.000906 | 0.000167 | 0.016529 |
| gbc26 | 7.51E−05 | 0 | 6.75E−05 | 9.33E−05 | 0.00057 | 0.00018 | 0 | 0 | 0.001515 | 0.000315 |
| gbc27 | 0 | 0 | 0 | 0.000381 | 0 | 6.80E−05 | 0 | 0.015655 | 0.006555 | 0.000179 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | TMEM106B | TMEM11 | TMEM164 | TNFAIP8 | TOMM40L | TOMM70 | TPI1P1 | TPST2 | TRAPPC5 | TRMT1L |
|---|---|---|---|---|---|---|---|---|---|---|
| rf0 | 0.01039 | 0 | 0.000759 | 0.005493 | 0.002176 | 3.94E−18 | 0.003985 | 0.003333 | 0 | 0.003598 |
| rf1 | 0.003515 | 0.001656 | 0.000455 | 7.83E−05 | 0.001674 | 0 | 0.009518 | 0.001443 | 2.91E−17 | 0 |
| rf2 | 0.006988 | 4.06E−19 | 0.000358 | 0.003842 | 0.003027 | 0.003497 | 0 | 0.000758 | 0.001 | 0.000876 |
| rf3 | 0.01138 | 0.000329 | 9.36E−19 | 0.002581 | 0 | 0 | 0.010496 | 0 | 0.006512 | 0.001156 |
| rf4 | 0.003755 | 0 | 0.000293 | 0 | 0 | 0.002569 | 0.006879 | 0 | 0 | 0 |
| rf5 | 0 | 0.000653 | 0.004502 | 0.000595 | 0.003122 | 0 | 0.000874 | 0 | 0.006792 | 0 |
| rf6 | 0.006441 | 0.002475 | 1.04E−17 | 0.000426 | 2.45E−19 | 0 | 0.003885 | 0.003006 | 0.003105 | 0 |
| rf7 | 0.001086 | 0.000865 | 0.004914 | 0.000409 | 0 | 0.001838 | 0.003473 | 0.001223 | 0.006607 | 0 |
| rf8 | 0 | 0.001253 | 0.004413 | 0.001837 | 0.003191 | 0.005306 | 0.005155 | 0 | 0.000642 | 0.001217 |
| rf9 | 0.006517 | 0.000629 | 0.000439 | 0 | 0.003155 | 0.003072 | 0.011202 | 0 | 0.001324 | 0.001779 |
| rf10 | 0.007982 | 0.005838 | 0 | 0 | 0 | 0.000841 | 1.63E−17 | 0.004815 | 0 | 0.000351 |
| rf11 | 0.007293 | 0 | 0.000563 | 0.000671 | 0.002842 | 0.003135 | 0.008868 | 0.000357 | 0.005516 | 0.000285 |
| rf12 | 0.003739 | 0 | 0 | 6.17E−18 | 0 | 0 | 0.00107 | 0.002482 | 0 | 0 |
| rf13 | 0.003112 | 0.000624 | 0.004817 | 0 | 0.002599 | 0.000729 | 0.004728 | 9.57E−18 | 0.001227 | 0.002735 |
| rf14 | 0.01039 | 0 | 0.000759 | 0.005493 | 0.002176 | 3.94E−18 | 0.003985 | 0.003333 | 0 | 0.003598 |
| rf15 | 0.003515 | 0.001656 | 0.000455 | 7.83E−05 | 0.001674 | 0 | 0.009518 | 0.001443 | 2.91E−17 | 0 |
| rf16 | 0.006988 | 4.06E−19 | 0.000358 | 0.003842 | 0.003027 | 0.003497 | 0 | 0.000758 | 0.001 | 0.000876 |
| rf17 | 0.01138 | 0.000329 | 9.36E−19 | 0.002581 | 0 | 0 | 0.010496 | 0 | 0.006512 | 0.001156 |
| rf18 | 0.003755 | 0 | 0.000293 | 0 | 0 | 0.002569 | 0.006879 | 0 | 0 | 0 |
| rf19 | 0 | 0.000653 | 0.004502 | 0.000595 | 0.003122 | 0 | 0.000874 | 0 | 0.006792 | 0 |
| rf20 | 0.006441 | 0.002475 | 1.04E−17 | 0.000426 | 2.45E−19 | 0 | 0.003885 | 0.003006 | 0.003105 | 0 |
| rf21 | 0.001086 | 0.000865 | 0.004914 | 0.000409 | 0 | 0.001838 | 0.003473 | 0.001223 | 0.006607 | 0 |
| rf22 | 0 | 0.001253 | 0.004413 | 0.001837 | 0.003191 | 0.005306 | 0.005155 | 0 | 0.000642 | 0.001217 |
| rf23 | 0.006517 | 0.000629 | 0.000439 | 0 | 0.003155 | 0.003072 | 0.011202 | 0 | 0.001324 | 0.001779 |
| rf24 | 0.007982 | 0.005838 | 0 | 0 | 0 | 0.000841 | 1.63E−17 | 0.004815 | 0 | 0.000351 |
| rf25 | 0.007293 | 0 | 0.000563 | 0.000671 | 0.002842 | 0.003135 | 0.008868 | 0.000357 | 0.005516 | 0.000285 |
| rf26 | 0.003739 | 0 | 0 | 6.17E−18 | 0 | 0 | 0.00107 | 0.002482 | 0 | 0 |
| rf27 | 0.003112 | 0.000624 | 0.004817 | 0 | 0.002599 | 0.000729 | 0.004728 | 9.57E−18 | 0.001227 | 0.002735 |
| extraTrees0 | 0.001112 | 0.000238 | 0.003124 | 0.000333 | 0.002817 | 0.001104 | 0.004888 | 0.000604 | 0.001661 | 0.001375 |
| extraTrees1 | 0.004746 | 0.0025 | 0.001185 | 0.000907 | 0.001859 | 0.00119 | 0.002933 | 0.002142 | 0.006314 | 0.001644 |
| extraTrees2 | 0.001354 | 0.001004 | 0.002634 | 0.001323 | 0.001728 | 0.003579 | 0.002007 | 0.000774 | 0.003175 | 0 |
| extraTrees3 | 0.004374 | 0.003734 | 2.00E−18 | 0.001034 | 0.001624 | 0.002492 | 0.004393 | 0.000501 | 0.001955 | 0.000903 |
| extraTrees4 | 0.001758 | 0.000238 | 0.001378 | 0.000796 | 0.001926 | 0.002871 | 0.001305 | 0.000759 | 2.73E−18 | 3.51E−19 |
| extraTrees5 | 0.003353 | 0.003452 | 0.000235 | 0.001109 | 0.000816 | 0.001006 | 0.001288 | 0.00228 | 0.00028 | 0.000982 |
| extraTrees6 | 0.003314 | 0.002193 | 0.000446 | 0 | 0.002014 | 7.01E−19 | 0.001482 | 0.001206 | 0.001912 | 0.002758 |
| extraTrees7 | 0.002846 | 0.002338 | 0.000858 | 1.09E−18 | 0.00348 | 0.003134 | 0.003604 | 2.49E−05 | 6.29E−17 | 0.001859 |
| extraTrees8 | 0.0022 | 0.001971 | 0.001668 | 0.000238 | 0.001976 | 0.001791 | 0.000639 | 0.00049 | 0.002732 | 0.001477 |
| extraTrees9 | 0.003361 | 0.00233 | 0.002145 | 6.09E−05 | 0.002947 | 0.001261 | 0.002972 | 0.001104 | 0.003087 | 0 |
| extraTrees10 | 0.002738 | 0.002074 | 0.001293 | 0.000538 | 0.004796 | 0.001469 | 0.000903 | 0.001152 | 0.002674 | 0.000975 |
| extraTrees11 | 0.006399 | 0.001035 | 4.10E−19 | 0.000625 | 0.002385 | 0.001458 | 0.003576 | 0.001564 | 0.000821 | 0.000468 |
| extraTrees12 | 0.001371 | 0.003241 | 0.002404 | 0.000535 | 2.73E−19 | 0.001292 | 0.002321 | 0.000286 | 0.003571 | 0.000234 |
| extraTrees13 | 0.002731 | 0.000846 | 0.001877 | 0.001007 | 0.00311 | 0.001204 | 0.001777 | 0.000627 | 0 | 0.000806 |
| extraTrees14 | 0.001112 | 0.000238 | 0.003124 | 0.000333 | 0.002817 | 0.001104 | 0.004888 | 0.000604 | 0.001661 | 0.001375 |
| extraTrees15 | 0.004746 | 0.0025 | 0.001185 | 0.000907 | 0.001859 | 0.00119 | 0.002933 | 0.002142 | 0.006314 | 0.001644 |
| extraTrees16 | 0.001354 | 0.001004 | 0.002634 | 0.001323 | 0.001728 | 0.003579 | 0.002007 | 0.000774 | 0.003175 | 0 |
| extraTrees17 | 0.004374 | 0.003734 | 2.00E−18 | 0.001034 | 0.001624 | 0.002492 | 0.004393 | 0.000501 | 0.001955 | 0.000903 |
| extraTrees18 | 0.001758 | 0.000238 | 0.001378 | 0.000796 | 0.001926 | 0.002871 | 0.001305 | 0.000759 | 2.73E−18 | 3.51E−19 |
| extraTrees19 | 0.003353 | 0.003452 | 0.000235 | 0.001109 | 0.000816 | 0.001006 | 0.001288 | 0.00228 | 0.00028 | 0.000982 |
| extraTrees20 | 0.003314 | 0.002193 | 0.000446 | 0 | 0.002014 | 7.01E−19 | 0.001482 | 0.001206 | 0.001912 | 0.002758 |
| extraTrees21 | 0.002846 | 0.002338 | 0.000858 | 1.09E−18 | 0.00348 | 0.003134 | 0.003604 | 2.49E−05 | 6.29E−17 | 0.001859 |
| extraTrees22 | 0.0022 | 0.001971 | 0.001668 | 0.000238 | 0.001976 | 0.001791 | 0.000639 | 0.00049 | 0.002732 | 0.001477 |
| extraTrees23 | 0.003361 | 0.00233 | 0.002145 | 6.09E−05 | 0.002947 | 0.001261 | 0.002972 | 0.001104 | 0.003087 | 0 |
| extraTrees24 | 0.002738 | 0.002074 | 0.001293 | 0.000538 | 0.004796 | 0.001469 | 0.000903 | 0.001152 | 0.002674 | 0.000975 |
| extraTrees25 | 0.006399 | 0.001035 | 4.10E−19 | 0.000625 | 0.002385 | 0.001458 | 0.003576 | 0.001564 | 0.000821 | 0.000468 |
| extraTrees26 | 0.001371 | 0.003241 | 0.002404 | 0.000535 | 2.73E−19 | 0.001292 | 0.002321 | 0.000286 | 0.003571 | 0.000234 |
| extraTrees27 | 0.002731 | 0.000846 | 0.001877 | 0.001007 | 0.00311 | 0.001204 | 0.001777 | 0.000627 | 0 | 0.000806 |
| gbc0 | 1.11E−06 | 0 | 9.53E−05 | 0 | 0 | 0.002307 | 0.026571 | 0 | 0.000891 | 0 |
| gbc1 | 6.90E−05 | 0.001665 | 0 | 0 | 6.53E−07 | 6.64E−05 | 0 | 0 | 0.056701 | 0 |
| gbc2 | 0 | 3.49E−06 | 0 | 0.01717 | 0 | 4.07E−05 | 0 | 6.31E−05 | 0 | 0 |
| gbc3 | 0.000177 | 0.002755 | 0 | 0 | 0 | 0.011373 | 0 | 0 | 0.038946 | 0 |
| gbc4 | 0.002618 | 2.24E−05 | 0 | 0 | 0.001453 | 0.001286 | 0 | 0 | 0 | 1.50E−05 |
| gbc5 | 0 | 0 | 0 | 0 | 4.72E−08 | 0.000458 | 0 | 1.96E−06 | 0 | 0 |
| gbc6 | 0 | 0.000132 | 0 | 0 | 0.000117 | 0.000313 | 0 | 0 | 0 | 0 |
| gbc7 | 0.0023 | 0.00044 | 7.89E−05 | 0 | 9.75E−07 | 0.002684 | 0 | 0 | 0.000376 | 0.00049 |
| gbc8 | 0 | 8.20E−05 | 0 | 0 | 0 | 0 | 0.001093 | 1.99E−05 | 0.013755 | 0 |
| gbc9 | 2.77E−07 | 0.000445 | 0 | 0.000244 | 4.77E−06 | 0.004712 | 0.000113 | 0 | 0 | 0.000177 |
| gbc10 | 0 | 0.000331 | 0.001225 | 0.00192 | 0 | 0.000206 | 3.84E−05 | 3.65E−06 | 0 | 0 |
| gbc11 | 0 | 0 | 0 | 0.009544 | 0.00026 | 0.005817 | 0 | 0 | 0.028132 | 0 |
| gbc12 | 0.000516 | 0.000221 | 0 | 0 | 0 | 0.005205 | 0 | 9.20E−06 | 6.11E−05 | 0 |
| gbc13 | 0.000489 | 0.000472 | 0 | 0 | 0 | 0 | 0.00302 | 0.005313 | 0 | 7.00E−06 |
| gbc14 | 1.11E−06 | 0 | 9.53E−05 | 0 | 0 | 0.002307 | 0.026571 | 0 | 0.000891 | 0 |
| gbc15 | 6.90E−05 | 0.001665 | 0 | 0 | 6.53E−07 | 6.64E−05 | 0 | 0 | 0.056701 | 0 |
| gbc16 | 0 | 3.49E−06 | 0 | 0.01717 | 0 | 4.07E−05 | 0 | 6.31E−05 | 0 | 0 |
| gbc17 | 0.000177 | 0.002755 | 0 | 0 | 0 | 0.011373 | 0 | 0 | 0.038946 | 0 |
| gbc18 | 0.002618 | 2.24E−05 | 0 | 0 | 0.001453 | 0.001286 | 0 | 0 | 0 | 1.50E−05 |
| gbc19 | 0 | 0 | 0 | 0 | 4.72E−08 | 0.000458 | 0 | 1.96E−06 | 0 | 0 |
| gbc20 | 0 | 0.000132 | 0 | 0 | 0.000117 | 0.000313 | 0 | 0 | 0 | 0 |
| gbc21 | 0.0023 | 0.00044 | 7.89E−05 | 0 | 9.75E−07 | 0.002684 | 0 | 0 | 0.000376 | 0.00049 |
| gbc22 | 0 | 8.20E−05 | 0 | 0 | 0 | 0 | 0.001093 | 1.99E−05 | 0.013755 | 0 |
| gbc23 | 2.77E−07 | 0.000445 | 0 | 0.000244 | 4.77E−06 | 0.004712 | 0.000113 | 0 | 0 | 0.000177 |
| gbc24 | 0 | 0.000331 | 0.001225 | 0.00192 | 0 | 0.000206 | 3.84E−05 | 3.65E−06 | 0 | 0 |
| gbc25 | 0 | 0 | 0 | 0.009544 | 0.00026 | 0.005817 | 0 | 0 | 0.028132 | 0 |
| gbc26 | 0.000516 | 0.000221 | 0 | 0 | 0 | 0.005205 | 0 | 9.20E−06 | 6.11E−05 | 0 |
| gbc27 | 0.000489 | 0.000472 | 0 | 0 | 0 | 0 | 0.00302 | 0.005313 | 0 | 7.00E−06 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | TRPC2 | TTI2 | U2SURP | UBE2Z | UBE3B | UFM1 | UHRF1BP1 | USP15 | USP39 | UTRN |
|---|---|---|---|---|---|---|---|---|---|---|
| rf0 | 7.94E−05 | 0.001212 | 0 | 0.001189 | 0.018854 | 0.002154 | 0 | 0.000724 | 0.003585 | 0.00038 |
| rf1 | 0 | 0 | 0.002644 | 0.000376 | 0.008301 | 0 | 0.004143 | 0 | 0.000768 | 0.00185 |
| rf2 | 0.005337 | 0.001147 | 0.007175 | 0.00115 | 0.015546 | 0.001304 | 0 | 0 | 0.010621 | 0.004267 |
| rf3 | 0.004766 | 0.005918 | 0.000745 | 0.004783 | 0.001064 | 0 | 0.001542 | 0 | 0.006153 | 0 |
| rf4 | 0.001891 | 0.000658 | 0.010206 | 0 | 0.01155 | 0 | 0.000651 | 0.00034 | 0.000693 | 8.76E−19 |
| rf5 | 0.000683 | 0.001848 | 0.000652 | 0 | 0.009287 | 4.57E−19 | 0.002886 | 0.001095 | 0.000768 | 0 |
| rf6 | 0.003496 | 0.002556 | 0 | 0.001106 | 0.001056 | 0 | 0.003827 | 2.56E−18 | 0.000354 | 0.007455 |
| rf7 | 0.001189 | 0.005058 | 0.001422 | 0 | 0.034414 | 0 | 0.004858 | 5.95E−18 | 0.000495 | 0.007872 |
| rf8 | 0.005566 | 0.00044 | 0 | 0.001986 | 0.004479 | 0 | 0.003195 | 0.002398 | 0.00209 | 0.000339 |
| rf9 | 0.006026 | 0.002772 | 0.000336 | 0 | 0.005437 | 9.14E−19 | 0 | 3.92E−17 | 1.56E−18 | 0.005857 |
| rf10 | 0.001685 | 0 | 0.006061 | 0 | 0.008773 | 0.002383 | 0.000961 | 0.002194 | 0.001293 | 0.002304 |
| rf11 | 0.001224 | 0.000726 | 2.72E−05 | 6.71E−19 | 0.009815 | 0.001577 | 0.001357 | 0 | 0.000281 | 1.65E−18 |
| rf12 | 0 | 0.012112 | 0.000567 | 0 | 0.012145 | 0.001224 | 0.004906 | 0.001944 | 4.31E−19 | 0.003851 |
| rf13 | 0.000877 | 0.00112 | 0.002498 | 0.00176 | 0.010007 | 0.000759 | 0.001656 | 0.001518 | 0.004257 | 0.0098 |
| rf14 | 7.94E−05 | 0.001212 | 0 | 0.001189 | 0.018854 | 0.002154 | 0 | 0.000724 | 0.003585 | 0.00038 |
| rf15 | 0 | 0 | 0.002644 | 0.000376 | 0.008301 | 0 | 0.004143 | 0 | 0.000768 | 0.00185 |
| rf16 | 0.005337 | 0.001147 | 0.007175 | 0.00115 | 0.015546 | 0.001304 | 0 | 0 | 0.010621 | 0.004267 |
| rf17 | 0.004766 | 0.005918 | 0.000745 | 0.004783 | 0.001064 | 0 | 0.001542 | 0 | 0.006153 | 0 |
| rf18 | 0.001891 | 0.000658 | 0.010206 | 0 | 0.01155 | 0 | 0.000651 | 0.00034 | 0.000693 | 8.76E−19 |
| rf19 | 0.000683 | 0.001848 | 0.000652 | 0 | 0.009287 | 4.57E−19 | 0.002886 | 0.001095 | 0.000768 | 0 |
| rf20 | 0.003496 | 0.002556 | 0 | 0.001106 | 0.001056 | 0 | 0.003827 | 2.56E−18 | 0.000354 | 0.007455 |
| rf21 | 0.001189 | 0.005058 | 0.001422 | 0 | 0.034414 | 0 | 0.004858 | 5.95E−18 | 0.000495 | 0.007872 |
| rf22 | 0.005566 | 0.00044 | 0 | 0.001986 | 0.004479 | 0 | 0.003195 | 0.002398 | 0.00209 | 0.000339 |
| rf23 | 0.006026 | 0.002772 | 0.000336 | 0 | 0.005437 | 9.14E−19 | 0 | 3.92E−17 | 1.56E−18 | 0.005857 |
| rf24 | 0.001685 | 0 | 0.006061 | 0 | 0.008773 | 0.002383 | 0.000961 | 0.002194 | 0.001293 | 0.002304 |
| rf25 | 0.001224 | 0.000726 | 2.72E−05 | 6.71E−19 | 0.009815 | 0.001577 | 0.001357 | 0 | 0.000281 | 1.65E−18 |
| rf26 | 0 | 0.012112 | 0.000567 | 0 | 0.012145 | 0.001224 | 0.004906 | 0.001944 | 4.31E−19 | 0.003851 |
| rf27 | 0.000877 | 0.00112 | 0.002498 | 0.00176 | 0.010007 | 0.000759 | 0.001656 | 0.001518 | 0.004257 | 0.0098 |
| extraTrees0 | 0.001516 | 0.001932 | 0.000545 | 0.002222 | 0.004376 | 0.001818 | 0.002382 | 0 | 0.002934 | 0.001579 |
| extraTrees1 | 0.001913 | 0.003255 | 0.002725 | 0.001077 | 0.007285 | 0.000809 | 0.004704 | 0.000273 | 0.001587 | 0.004388 |
| extraTrees2 | 0.001741 | 0.005785 | 0.002962 | 0.001108 | 0.001345 | 0.001199 | 0.004473 | 0.000843 | 0.001388 | 0.005469 |
| extraTrees3 | 0.001953 | 0.001888 | 0.00141 | 0.000792 | 0.007623 | 0.001466 | 0.003753 | 0.00144 | 0.001643 | 0.003836 |
| extraTrees4 | 0.000576 | 0.003857 | 0.001776 | 0.000336 | 0.006848 | 0.001112 | 0.002127 | 0.003264 | 0.003847 | 0.002834 |
| extraTrees5 | 0 | 0.004812 | 0 | 0.000742 | 0.004747 | 0.000555 | 0.004192 | 0.001584 | 0.003996 | 0.004431 |
| extraTrees6 | 0.000857 | 0.000601 | 0.001313 | 0.002161 | 0.002455 | 0.00077 | 0.004969 | 0.001132 | 0.002355 | 0.00134 |
| extraTrees7 | 0.002183 | 0.003022 | 5.98E−18 | 0.00153 | 0.005118 | 0.000563 | 0.001686 | 0.001439 | 0.002549 | 0.00264 |
| extraTrees8 | 0.002437 | 0.00306 | 0.000589 | 0.00146 | 0.002889 | 0.000989 | 0.004903 | 0.000618 | 0.001435 | 0.002484 |
| extraTrees9 | 0.002219 | 0.001966 | 0.002498 | 0.001547 | 0.000905 | 0.000267 | 2.12E−18 | 0.000662 | 0.001806 | 0.003893 |
| extraTrees10 | 0.002411 | 0.003414 | 0.004581 | 0.003031 | 0.003749 | 0.000253 | 0.001827 | 0.000657 | 0.001973 | 0.003589 |
| extraTrees11 | 0.00166 | 0.00221 | 0.002703 | 0.001104 | 0.009236 | 0.000839 | 0.00156 | 0.00028 | 0.003066 | 0.006051 |
| extraTrees12 | 0.00224 | 0.004038 | 0.000823 | 0.000297 | 0.003526 | 5.93E−17 | 0.003784 | 0 | 0.002273 | 0.004575 |
| extraTrees13 | 0.000464 | 0.001596 | 0.000932 | 0.001351 | 0.004876 | 0.001006 | 0.006056 | 5.14E−17 | 0.002472 | 0.011798 |
| extraTrees14 | 0.001516 | 0.001932 | 0.000545 | 0.002222 | 0.004376 | 0.001818 | 0.002382 | 0 | 0.002934 | 0.001579 |
| extraTrees15 | 0.001913 | 0.003255 | 0.002725 | 0.001077 | 0.007285 | 0.000809 | 0.004704 | 0.000273 | 0.001587 | 0.004388 |
| extraTrees16 | 0.001741 | 0.005785 | 0.002962 | 0.001108 | 0.001345 | 0.001199 | 0.004473 | 0.000843 | 0.001388 | 0.005469 |
| extraTrees17 | 0.001953 | 0.001888 | 0.00141 | 0.000792 | 0.007623 | 0.001466 | 0.003753 | 0.00144 | 0.001643 | 0.003836 |
| extraTrees18 | 0.000576 | 0.003857 | 0.001776 | 0.000336 | 0.006848 | 0.001112 | 0.002127 | 0.003264 | 0.003847 | 0.002834 |
| extraTrees19 | 0 | 0.004812 | 0 | 0.000742 | 0.004747 | 0.000555 | 0.004192 | 0.001584 | 0.003996 | 0.004431 |
| extraTrees20 | 0.000857 | 0.000601 | 0.001313 | 0.002161 | 0.002455 | 0.00077 | 0.004969 | 0.001132 | 0.002355 | 0.00134 |
| extraTrees21 | 0.002183 | 0.003022 | 5.98E−18 | 0.00153 | 0.005118 | 0.000563 | 0.001686 | 0.001439 | 0.002549 | 0.00264 |
| extraTrees22 | 0.002437 | 0.00306 | 0.000589 | 0.00146 | 0.002889 | 0.000989 | 0.004903 | 0.000618 | 0.001435 | 0.002484 |
| extraTrees23 | 0.002219 | 0.001966 | 0.002498 | 0.001547 | 0.000905 | 0.000267 | 2.12E−18 | 0.000662 | 0.001806 | 0.003893 |
| extraTrees24 | 0.002411 | 0.003414 | 0.004581 | 0.003031 | 0.003749 | 0.000253 | 0.001827 | 0.000657 | 0.001973 | 0.003589 |
| extraTrees25 | 0.00166 | 0.00221 | 0.002703 | 0.001104 | 0.009236 | 0.000839 | 0.00156 | 0.00028 | 0.003066 | 0.006051 |
| extraTrees26 | 0.00224 | 0.004038 | 0.000823 | 0.000297 | 0.003526 | 5.93E−17 | 0.003784 | 0 | 0.002273 | 0.004575 |
| extraTrees27 | 0.000464 | 0.001596 | 0.000932 | 0.001351 | 0.004876 | 0.001006 | 0.006056 | 5.14E−17 | 0.002472 | 0.011798 |
| gbc0 | 0 | 0.003025 | 0 | 0 | 0 | 0 | 0 | 0.007104 | 0.003333 | 0.01037 |
| gbc1 | 1.65E−05 | 0 | 0 | 5.54E−05 | 0 | 0 | 0 | 9.98E−06 | 0.016246 | 0 |
| gbc2 | 0.000106 | 0.024255 | 6.12E−05 | 0 | 0 | 0.000151 | 0 | 0.007388 | 0 | 0 |
| gbc3 | 0.000492 | 5.03E−06 | 0 | 0 | 0.002568 | 2.80E−05 | 0.000864 | 0.000166 | 0.000124 | 0 |
| gbc4 | 0 | 0.000381 | 0.012842 | 0.000235 | 0.009733 | 8.69E−07 | 0 | 9.29E−05 | 0 | 0.000192 |
| gbc5 | 0 | 0.010103 | 0 | 0 | 0.028278 | 0 | 0.007522 | 0 | 0 | 0 |
| gbc6 | 0 | 0.000323 | 0.000121 | 0 | 0.008761 | 0 | 0.004613 | 0 | 0.000315 | 0 |
| gbc7 | 0.000725 | 0 | 0 | 0.000284 | 0.046641 | 0 | 0.000181 | 0 | 0 | 0 |
| gbc8 | 0 | 0 | 0 | 0 | 0.009213 | 4.53E−06 | 5.58E−05 | 0 | 0 | 0 |
| gbc9 | 0 | 0.000511 | 0 | 1.72E−06 | 0.000101 | 0 | 0.008176 | 0.001583 | 0.000287 | 0 |
| gbc10 | 1.74E−05 | 0.010143 | 5.19E−05 | 2.04E−05 | 0 | 0 | 0.000195 | 0 | 0.023613 | 0 |
| gbc11 | 0.000184 | 1.32E−06 | 0.000433 | 0.010477 | 0.018667 | 0 | 0.007909 | 0 | 0 | 0 |
| gbc12 | 4.16E−05 | 0 | 0 | 0 | 0 | 2.27E−05 | 0.003014 | 0 | 2.53E−06 | 9.89E−06 |
| gbc13 | 0 | 2.35E−07 | 0.000817 | 0 | 0 | 9.56E−05 | 0.001088 | 0.000493 | 0 | 0.000261 |
| gbc14 | 0 | 0.003025 | 0 | 0 | 0 | 0 | 0 | 0.007104 | 0.003333 | 0.01037 |
| gbc15 | 1.65E−05 | 0 | 0 | 5.54E−05 | 0 | 0 | 0 | 9.98E−06 | 0.016246 | 0 |
| gbc16 | 0.000106 | 0.024255 | 6.12E−05 | 0 | 0 | 0.000151 | 0 | 0.007388 | 0 | 0 |
| gbc17 | 0.000492 | 5.03E−06 | 0 | 0 | 0.002568 | 2.80E−05 | 0.000864 | 0.000166 | 0.000124 | 0 |
| gbc18 | 0 | 0.000381 | 0.012842 | 0.000235 | 0.009733 | 8.69E−07 | 0 | 9.29E−05 | 0 | 0.000192 |
| gbc19 | 0 | 0.010103 | 0 | 0 | 0.028278 | 0 | 0.007522 | 0 | 0 | 0 |
| gbc20 | 0 | 0.000323 | 0.000121 | 0 | 0.008761 | 0 | 0.004613 | 0 | 0.000315 | 0 |
| gbc21 | 0.000725 | 0 | 0 | 0.000284 | 0.046641 | 0 | 0.000181 | 0 | 0 | 0 |
| gbc22 | 0 | 0 | 0 | 0 | 0.009213 | 4.53E−06 | 5.58E−05 | 0 | 0 | 0 |
| gbc23 | 0 | 0.000511 | 0 | 1.72E−06 | 0.000101 | 0 | 0.008176 | 0.001583 | 0.000287 | 0 |
| gbc24 | 1.74E−05 | 0.010143 | 5.19E−05 | 2.04E−05 | 0 | 0 | 0.000195 | 0 | 0.023613 | 0 |
| gbc25 | 0.000184 | 1.32E−06 | 0.000433 | 0.010477 | 0.018667 | 0 | 0.007909 | 0 | 0 | 0 |
| gbc26 | 4.16E−05 | 0 | 0 | 0 | 0 | 2.27E−05 | 0.003014 | 0 | 2.53E−06 | 9.89E−06 |
| gbc27 | 0 | 2.35E−07 | 0.000817 | 0 | 0 | 9.56E−05 | 0.001088 | 0.000493 | 0 | 0.000261 |
| ada0 | 0.02 | 0 | 0.02 | 0 | 0 | 0 | 0 | 0 | 0 | 0 |
| ada1 | 0.02 | 0 | 0.02 | 0 | 0 | 0 | 0 | 0 | 0 | 0 |
| ada2 | 0.02 | 0 | 0.02 | 0 | 0 | 0 | 0 | 0 | 0 | 0 |
| ada3 | 0.02 | 0 | 0.02 | 0 | 0 | 0 | 0 | 0 | 0 | 0 |
| ada4 | 0.02 | 0 | 0.02 | 0 | 0 | 0 | 0 | 0 | 0 | 0 |
| ada5 | 0.02 | 0 | 0.02 | 0 | 0 | 0 | 0 | 0 | 0 | 0 |
| ada6 | 0.02 | 0 | 0.02 | 0 | 0 | 0 | 0 | 0 | 0 | 0 |
| ada7 | 0.02 | 0 | 0.02 | 0 | 0 | 0 | 0 | 0 | 0 | 0 |
| ada8 | 0.02 | 0 | 0.02 | 0 | 0 | 0 | 0 | 0 | 0 | 0 |
| ada9 | 0.02 | 0 | 0.02 | 0 | 0 | 0 | 0 | 0 | 0 | 0 |
| ada10 | 0.02 | 0 | 0.02 | 0 | 0 | 0 | 0 | 0 | 0 | 0 |
| ada11 | 0.02 | 0 | 0.02 | 0 | 0 | 0 | 0 | 0 | 0 | 0 |
| ada12 | 0.02 | 0 | 0.02 | 0 | 0 | 0 | 0 | 0 | 0 | 0 |
| ada13 | 0.02 | 0 | 0.02 | 0 | 0 | 0 | 0 | 0 | 0 | 0 |
| ada14 | 0.02 | 0 | 0.02 | 0 | 0 | 0 | 0 | 0 | 0 | 0 |
| ada15 | 0.02 | 0 | 0.02 | 0 | 0 | 0 | 0 | 0 | 0 | 0 |
| ada16 | 0.02 | 0 | 0.02 | 0 | 0 | 0 | 0 | 0 | 0 | 0 |
| ada17 | 0.02 | 0 | 0.02 | 0 | 0 | 0 | 0 | 0 | 0 | 0 |
| ada18 | 0.02 | 0 | 0.02 | 0 | 0 | 0 | 0 | 0 | 0 | 0 |
| ada19 | 0.02 | 0 | 0.02 | 0 | 0 | 0 | 0 | 0 | 0 | 0 |
| ada20 | 0.02 | 0 | 0.02 | 0 | 0 | 0 | 0 | 0 | 0 | 0 |
| ada21 | 0.02 | 0 | 0.02 | 0 | 0 | 0 | 0 | 0 | 0 | 0 |
| ada22 | 0.02 | 0 | 0.02 | 0 | 0 | 0 | 0 | 0 | 0 | 0 |
| ada23 | 0.02 | 0 | 0.02 | 0 | 0 | 0 | 0 | 0 | 0 | 0 |
| ada24 | 0.02 | 0 | 0.02 | 0 | 0 | 0 | 0 | 0 | 0 | 0 |
| ada25 | 0.02 | 0 | 0.02 | 0 | 0 | 0 | 0 | 0 | 0 | 0 |
| ada26 | 0.02 | 0 | 0.02 | 0 | 0 | 0 | 0 | 0 | 0 | 0 |
| ada27 | 0.02 | 0 | 0.02 | 0 | 0 | 0 | 0 | 0 | 0 | 0 |

| Gene | UXS1 | VKORC1 | VPS13C | VPS45 | VRK1 | WASF1 | WDR1 | WDR13 | WDR47 | WWC3 |
|---|---|---|---|---|---|---|---|---|---|---|
| rf0 | 0.002602 | 0.002122 | 0.000376 | 0.000866 | 1.66E−18 | 0 | 0.002035 | 0.001065 | 0.006414 | 0.001493 |
| rf1 | 0.001023 | 0 | 0.000729 | 0 | 0.008142 | 0.004079 | 0 | 0.000665 | 0.000388 | 0.000854 |
| rf2 | 0.006861 | 0.005117 | 0.003166 | 0.00066 | 0.006053 | 0.007321 | 0.000279 | 0 | 0.003269 | 0 |
| rf3 | 0.001809 | 0 | 0.008751 | 1.56E−18 | 0.001669 | 0.001472 | 0.000732 | 0 | 0.004396 | 0 |
| rf4 | 0 | 0.00112 | 4.53E−18 | 9.09E−18 | 7.32E−19 | 0.002127 | 0.00035 | 0 | 0.004544 | 0 |
| rf5 | 0.001335 | 0.002004 | 0.000797 | 0.000507 | 0.00112 | 0 | 0.002425 | 0.001151 | 0 | 6.86E−19 |
| rf6 | 0 | 0.010416 | 0 | 1.56E−18 | 0 | 0 | 0.000751 | 0 | 0.000953 | 0 |
| rf7 | 0 | 0.003952 | 0 | 0.003413 | 0.001736 | 0.002414 | 8.49E−18 | 0 | 0 | 0 |
| rf8 | 0.005285 | 0.001061 | 0.001266 | 0.002261 | 0.002278 | 0.001097 | 0.000792 | 0 | 0.00164 | 0 |
| rf9 | 0.000752 | 0 | 0.000635 | 8.01E−19 | 0.00146 | 0.000646 | 0 | 0.003211 | 0.005961 | 0.002608 |
| rf10 | 0 | 0.004794 | 0 | 0.00036 | 0.00685 | 0.003016 | 0.001015 | 0 | 0.006775 | 0.003538 |
| rf11 | 0.002297 | 0.000793 | 0.003017 | 0.000371 | 0.010547 | 0.00206 | 0.001435 | 0.001384 | 0.012566 | 0 |
| rf12 | 0.00183 | 0 | 0.001765 | 0.001682 | 0.003989 | 0 | 0.001148 | 0.001125 | 0.001446 | 0.00034 |
| rf13 | 0.000341 | 0 | 3.66E−18 | 0.001905 | 0.004397 | 0.001384 | 0.000687 | 0 | 0.009381 | 0 |
| rf14 | 0.002602 | 0.002122 | 0.000376 | 0.000866 | 1.66E−18 | 0 | 0.002035 | 0.001065 | 0.006414 | 0.001493 |
| rf15 | 0.001023 | 0 | 0.000729 | 0 | 0.008142 | 0.004079 | 0 | 0.000665 | 0.000388 | 0.000854 |
| rf16 | 0.006861 | 0.005117 | 0.003166 | 0.00066 | 0.006053 | 0.007321 | 0.000279 | 0 | 0.003269 | 0 |
| rf17 | 0.001809 | 0 | 0.008751 | 1.56E−18 | 0.001669 | 0.001472 | 0.000732 | 0 | 0.004396 | 0 |
| rf18 | 0 | 0.00112 | 4.53E−18 | 9.09E−18 | 7.32E−19 | 0.002127 | 0.00035 | 0 | 0.004544 | 0 |
| rf19 | 0.001335 | 0.002004 | 0.000797 | 0.000507 | 0.00112 | 0 | 0.002425 | 0.001151 | 0 | 6.86E−19 |
| rf20 | 0 | 0.010416 | 0 | 1.56E−18 | 0 | 0 | 0.000751 | 0 | 0.000953 | 0 |
| rf21 | 0 | 0.003952 | 0 | 0.003413 | 0.001736 | 0.002414 | 8.49E−18 | 0 | 0 | 0 |
| rf22 | 0.005285 | 0.001061 | 0.001266 | 0.002261 | 0.002278 | 0.001097 | 0.000792 | 0 | 0.00164 | 0 |
| rf23 | 0.000752 | 0 | 0.000635 | 8.01E−19 | 0.00146 | 0.000646 | 0 | 0.003211 | 0.005961 | 0.002608 |
| rf24 | 0 | 0.004794 | 0 | 0.00036 | 0.00685 | 0.003016 | 0.001015 | 0 | 0.006775 | 0.003538 |
| rf25 | 0.002297 | 0.000793 | 0.003017 | 0.000371 | 0.010547 | 0.00206 | 0.001435 | 0.001384 | 0.012566 | 0 |
| rf26 | 0.00183 | 0 | 0.001765 | 0.001682 | 0.003989 | 0 | 0.001148 | 0.001125 | 0.001446 | 0.00034 |
| rf27 | 0.000341 | 0 | 3.66E−18 | 0.001905 | 0.004397 | 0.001384 | 0.000687 | 0 | 0.009381 | 0 |
| extraTrees0 | 0.001504 | 0.002021 | 0.000584 | 0.002298 | 0.001771 | 0.001563 | 0.001132 | 0.001152 | 0.001996 | 0.003058 |
| extraTrees1 | 0.001435 | 0.001919 | 0.002507 | 0.000476 | 0.004221 | 0.003413 | 0.000571 | 0.00105 | 0.003164 | 0.001916 |
| extraTrees2 | 0.001971 | 0.006793 | 0.00347 | 0.001107 | 0.002415 | 0.0021 | 0.001734 | 7.16E−18 | 0.000846 | 0.00166 |
| extraTrees3 | 0.000947 | 0.003173 | 0.002461 | 0.000869 | 0.003891 | 0.002051 | 0.000856 | 0.000753 | 0.004702 | 0.000609 |
| extraTrees4 | 0.002725 | 0.00338 | 0.00414 | 0.001291 | 0.002283 | 0.001814 | 0.001758 | 0.000419 | 0.001563 | 0.004194 |
| extraTrees5 | 0.000816 | 0.005726 | 0.00097 | 0.001283 | 0.000102 | 0.003028 | 0.000238 | 0.003274 | 0.00399 | 0.000529 |
| extraTrees6 | 0.00094 | 0.001997 | 0.001649 | 0.000996 | 0.001736 | 0.00143 | 0.000235 | 0.00094 | 0.001533 | 0.001243 |
| extraTrees7 | 0.003673 | 0.001353 | 0.003818 | 0 | 0.00494 | 0.001431 | 1.14E−18 | 4.24E−18 | 0.003474 | 0.003661 |
| extraTrees8 | 0.001782 | 0.00113 | 0.004071 | 0.001841 | 0.005418 | 0.001684 | 0.001299 | 0.001812 | 1.26E−18 | 0.000715 |
| extraTrees9 | 0.002737 | 0.001953 | 0.002821 | 0.001108 | 0.005646 | 0.000908 | 0.001095 | 0.001093 | 0.002543 | 0.002602 |
| extraTrees10 | 0.001634 | 0.004123 | 0.001014 | 0.001012 | 0.00367 | 0.002786 | 0.000707 | 2.05E−19 | 0.001312 | 0.002488 |
| extraTrees11 | 0.003029 | 0.001648 | 0.00242 | 0.001081 | 0.005055 | 0.003009 | 0.000452 | 0.001255 | 0.002436 | 0.003862 |
| extraTrees12 | 0.001855 | 0.002673 | 0.001839 | 0.001407 | 0.001759 | 0.003669 | 0.000329 | 0.000469 | 0.002958 | 0.000941 |
| extraTrees13 | 0.002438 | 0.001104 | 0.001901 | 0.002272 | 0.001854 | 0.002694 | 0.00169 | 0.001854 | 0.000861 | 0.001563 |
| extraTrees14 | 0.001504 | 0.002021 | 0.000584 | 0.002298 | 0.001771 | 0.001563 | 0.001132 | 0.001152 | 0.001996 | 0.003058 |
| extraTrees15 | 0.001435 | 0.001919 | 0.002507 | 0.000476 | 0.004221 | 0.003413 | 0.000571 | 0.00105 | 0.003164 | 0.001916 |
| extraTrees16 | 0.001971 | 0.006793 | 0.00347 | 0.001107 | 0.002415 | 0.0021 | 0.001734 | 7.16E−18 | 0.000846 | 0.00166 |
| extraTrees17 | 0.000947 | 0.003173 | 0.002461 | 0.000869 | 0.003891 | 0.002051 | 0.000856 | 0.000753 | 0.004702 | 0.000609 |
| extraTrees18 | 0.002725 | 0.00338 | 0.00414 | 0.001291 | 0.002283 | 0.001814 | 0.001758 | 0.000419 | 0.001563 | 0.004194 |
| extraTrees19 | 0.000816 | 0.005726 | 0.00097 | 0.001283 | 0.000102 | 0.003028 | 0.000238 | 0.003274 | 0.00399 | 0.000529 |
| extraTrees20 | 0.00094 | 0.001997 | 0.001649 | 0.000996 | 0.001736 | 0.00143 | 0.000235 | 0.00094 | 0.001533 | 0.001243 |
| extraTrees21 | 0.003673 | 0.001353 | 0.003818 | 0 | 0.00494 | 0.001431 | 1.14E−18 | 4.24E−18 | 0.003474 | 0.003661 |
| extraTrees22 | 0.001782 | 0.00113 | 0.004071 | 0.001841 | 0.005418 | 0.001684 | 0.001299 | 0.001812 | 1.26E−18 | 0.000715 |
| extraTrees23 | 0.002737 | 0.001953 | 0.002821 | 0.001108 | 0.005646 | 0.000908 | 0.001095 | 0.001093 | 0.002543 | 0.002602 |
| extraTrees24 | 0.001634 | 0.004123 | 0.001014 | 0.001012 | 0.00367 | 0.002786 | 0.000707 | 2.05E−19 | 0.001312 | 0.002488 |
| extraTrees25 | 0.003029 | 0.001648 | 0.00242 | 0.001081 | 0.005055 | 0.003009 | 0.000452 | 0.001255 | 0.002436 | 0.003862 |
| extraTrees26 | 0.001855 | 0.002673 | 0.001839 | 0.001407 | 0.001759 | 0.003669 | 0.000329 | 0.000469 | 0.002958 | 0.000941 |
| extraTrees27 | 0.002438 | 0.001104 | 0.001901 | 0.002272 | 0.001854 | 0.002694 | 0.00169 | 0.001854 | 0.000861 | 0.001563 |
| gbc0 | 0.001042 | 0 | 0.002915 | 0.008281 | 0.009657 | 1.19E−05 | 6.60E−06 | 0 | 0 | 0.000246 |
| gbc1 | 0.00264 | 7.69E−06 | 0.000169 | 7.34E−05 | 0.00896 | 0 | 0 | 0.002184 | 1.74E−05 | 2.47E−06 |
| gbc2 | 0 | 0.016145 | 0 | 0.000874 | 0.037109 | 0.0005 | 0 | 0.000246 | 0.002597 | 0.01055 |
| gbc3 | 0 | 0 | 0 | 0 | 0.010193 | 0.000932 | 3.06E−05 | 0.000759 | 0.004168 | 0.00226 |
| gbc4 | 4.22E−05 | 0 | 0.00032 | 6.16E−05 | 0.000762 | 0.00245 | 0 | 0 | 0 | 8.10E−07 |
| gbc5 | 0.00022 | 0 | 0.008253 | 0 | 0.010671 | 7.07E−05 | 0.000345 | 2.78E−05 | 0.002242 | 0 |
| gbc6 | 4.65E−05 | 0 | 0.005268 | 3.05E−05 | 0.01634 | 0 | 0.000187 | 0 | 1.27E−05 | 0.0104 |
| gbc7 | 0.00325 | 0 | 0.000261 | 0 | 0.001995 | 0 | 0 | 0 | 0.007599 | 0 |
| gbc8 | 0 | 0 | 0.001185 | 0 | 0.005632 | 0.000819 | 0 | 0.019878 | 0.000388 | 2.58E−05 |
| gbc9 | 0.000267 | 0.000294 | 0.001319 | 5.13E−05 | 4.26E−05 | 0.002533 | 0.006274 | 0.000193 | 0.005392 | 0 |
| gbc10 | 0.005856 | 0.001579 | 0 | 5.33E−05 | 0 | 0.004233 | 0 | 1.10E−05 | 0.000161 | 9.01E−06 |
| gbc11 | 1.71E−05 | 6.45E−05 | 0.002273 | 0 | 0.000373 | 0 | 7.72E−06 | 0 | 0.000878 | 0.002334 |
| gbc12 | 0 | 0.023203 | 0.016309 | 0.000336 | 0 | 0 | 1.19E−06 | 0.012465 | 0.019518 | 0.003116 |
| gbc13 | 0 | 9.19E−06 | 0 | 0 | 0.005888 | 0.019426 | 0 | 0.00089 | 0.00341 | 0 |
| gbc14 | 0.001042 | 0 | 0.002915 | 0.008281 | 0.009657 | 1.19E−05 | 6.60E−06 | 0 | 0 | 0.000246 |
| gbc15 | 0.00264 | 7.69E−06 | 0.000169 | 7.34E−05 | 0.00896 | 0 | 0 | 0.002184 | 1.74E−05 | 2.47E−06 |
| gbc16 | 0 | 0.016145 | 0 | 0.000874 | 0.037109 | 0.0005 | 0 | 0.000246 | 0.002597 | 0.01055 |
| gbc17 | 0 | 0 | 0 | 0 | 0.010193 | 0.000932 | 3.06E−05 | 0.000759 | 0.004168 | 0.00226 |
| gbc18 | 4.22E−05 | 0 | 0.00032 | 6.16E−05 | 0.000762 | 0.00245 | 0 | 0 | 0 | 8.10E−07 |
| gbc19 | 0.00022 | 0 | 0.008253 | 0 | 0.010671 | 7.07E−05 | 0.000345 | 2.78E−05 | 0.002242 | 0 |
| gbc20 | 4.65E−05 | 0 | 0.005268 | 3.05E−05 | 0.01634 | 0 | 0.000187 | 0 | 1.27E−05 | 0.0104 |
| gbc21 | 0.00325 | 0 | 0.000261 | 0 | 0.001995 | 0 | 0 | 0 | 0.007599 | 0 |
| gbc22 | 0 | 0 | 0.001185 | 0 | 0.005632 | 0.000819 | 0 | 0.019878 | 0.000388 | 2.58E−05 |
| gbc23 | 0.000267 | 0.000294 | 0.001319 | 5.13E−05 | 4.26E−05 | 0.002533 | 0.006274 | 0.000193 | 0.005392 | 0 |
| gbc24 | 0.005856 | 0.001579 | 0 | 5.33E−05 | 0 | 0.004233 | 0 | 1.10E−05 | 0.000161 | 9.01E−06 |
| gbc25 | 1.71E−05 | 6.45E−05 | 0.002273 | 0 | 0.000373 | 0 | 7.72E−06 | 0 | 0.000878 | 0.002334 |
| gbc26 | 0 | 0.023203 | 0.016309 | 0.000336 | 0 | 0 | 1.19E−06 | 0.012465 | 0.019518 | 0.003116 |
| gbc27 | 0 | 9.19E−06 | 0 | 0 | 0.005888 | 0.019426 | 0 | 0.00089 | 0.00341 | 0 |
| ada0 | 0 | 0 | 0 | 0 | 0 | 0 | 0 | 0 | 0.02 | 0 |
| ada1 | 0 | 0 | 0 | 0 | 0 | 0 | 0 | 0 | 0.02 | 0 |
| ada2 | 0 | 0 | 0 | 0 | 0 | 0 | 0 | 0 | 0.02 | 0 |
| ada3 | 0 | 0 | 0 | 0 | 0 | 0 | 0 | 0 | 0.02 | 0 |
| ada4 | 0 | 0 | 0 | 0 | 0 | 0 | 0 | 0 | 0.02 | 0 |
| ada5 | 0 | 0 | 0 | 0 | 0 | 0 | 0 | 0 | 0.02 | 0 |
| ada6 | 0 | 0 | 0 | 0 | 0 | 0 | 0 | 0 | 0.02 | 0 |
| ada7 | 0 | 0 | 0 | 0 | 0 | 0 | 0 | 0 | 0.02 | 0 |
| ada8 | 0 | 0 | 0 | 0 | 0 | 0 | 0 | 0 | 0.02 | 0 |
| ada9 | 0 | 0 | 0 | 0 | 0 | 0 | 0 | 0 | 0.02 | 0 |
| ada10 | 0 | 0 | 0 | 0 | 0 | 0 | 0 | 0 | 0.02 | 0 |
| ada11 | 0 | 0 | 0 | 0 | 0 | 0 | 0 | 0 | 0.02 | 0 |
| ada12 | 0 | 0 | 0 | 0 | 0 | 0 | 0 | 0 | 0.02 | 0 |
| ada13 | 0 | 0 | 0 | 0 | 0 | 0 | 0 | 0 | 0.02 | 0 |
| ada14 | 0 | 0 | 0 | 0 | 0 | 0 | 0 | 0 | 0.02 | 0 |
| ada15 | 0 | 0 | 0 | 0 | 0 | 0 | 0 | 0 | 0.02 | 0 |
| ada16 | 0 | 0 | 0 | 0 | 0 | 0 | 0 | 0 | 0.02 | 0 |
| ada17 | 0 | 0 | 0 | 0 | 0 | 0 | 0 | 0 | 0.02 | 0 |
| ada18 | 0 | 0 | 0 | 0 | 0 | 0 | 0 | 0 | 0.02 | 0 |
| ada19 | 0 | 0 | 0 | 0 | 0 | 0 | 0 | 0 | 0.02 | 0 |
| ada20 | 0 | 0 | 0 | 0 | 0 | 0 | 0 | 0 | 0.02 | 0 |
| ada21 | 0 | 0 | 0 | 0 | 0 | 0 | 0 | 0 | 0.02 | 0 |
| ada22 | 0 | 0 | 0 | 0 | 0 | 0 | 0 | 0 | 0.02 | 0 |
| ada23 | 0 | 0 | 0 | 0 | 0 | 0 | 0 | 0 | 0.02 | 0 |
| ada24 | 0 | 0 | 0 | 0 | 0 | 0 | 0 | 0 | 0.02 | 0 |
| ada25 | 0 | 0 | 0 | 0 | 0 | 0 | 0 | 0 | 0.02 | 0 |
| ada26 | 0 | 0 | 0 | 0 | 0 | 0 | 0 | 0 | 0.02 | 0 |
| ada27 | 0 | 0 | 0 | 0 | 0 | 0 | 0 | 0 | 0.02 | 0 |
| Gene | XIAP | XRN1 | ZBTB1 | ZBTB4 | ZNF134 | ZNF33A | ZNF347 | ZNF44 | ZNF451 | ZNF517 |
| rf0 | 0.001927 | 0 | 0.002383 | 0.001066 | 0 | 0 | 0 | 0.006277 | 0 | 0.000343 |
| rf1 | 0 | 0.001471 | 0.000762 | 0.002801 | 0.000325 | 0.003226 | 0 | 0.002004 | 0.000357 | 0.001463 |
| rf2 | 0.000727 | 0 | 0.003647 | 0.000711 | 0 | 0.000507 | 0.003272 | 0.002137 | 0.004695 | 0.000783 |
| rf3 | 0 | 0.004502 | 0.001319 | 0.003271 | 0.000629 | 0 | 0 | 0 | 0.000347 | 0.003747 |
| rf4 | 6.59E−18 | 0.002015 | 0.002126 | 0.003123 | 0.002087 | 0.002816 | 0.003143 | 0.00339 | 0.004793 | 1.29E−18 |
| rf5 | 4.13E−05 | 0.001565 | 0.00105 | 6.97E−18 | 0.000716 | 0.001092 | 0 | 0.004621 | 0.000701 | 0.001205 |
| rf6 | 0.002622 | 0.002326 | 0.001549 | 0 | 0.004683 | 0 | 0.000854 | 0.004707 | 0.010033 | 0.001018 |
| rf7 | 0.00458 | 0.000861 | 0.000366 | 0.001745 | 0.002508 | 0.001463 | 0.003269 | 0.007573 | 0.001072 | 0.002297 |
| rf8 | 0.004472 | 0.000464 | 2.64E−18 | 3.14E−18 | 0 | 4.15E−18 | 0.001711 | 0.001438 | 0.00647 | 0.001137 |
| rf9 | 6.71E−19 | 0.005028 | 0.001682 | 0.003316 | 0.000789 | 0.002553 | 2.86E−18 | 0.011293 | 0 | 0.003144 |
| rf10 | 0.000697 | 0.003204 | 0.001941 | 0.002455 | 0 | 1.12E−18 | 0 | 0.004544 | 0.003003 | 0 |
| rf11 | 0.000391 | 0.00086 | 0.0039 | 0 | 0.005011 | 7.75E−05 | 0.000928 | 0.000645 | 7.99E−18 | 0.000354 |
| rf12 | 0 | 0.001195 | 0 | 0 | 0 | 0 | 0 | 0.003404 | 0 | 0.000638 |
| rf13 | 0.002493 | 0.001766 | 0 | 0 | 0.003236 | 0.001475 | 0 | 0.00566 | 0.001758 | 0.002722 |
| rf14 | 0.001927 | 0 | 0.002383 | 0.001066 | 0 | 0 | 0 | 0.006277 | 0 | 0.000343 |
| rf15 | 0 | 0.001471 | 0.000762 | 0.002801 | 0.000325 | 0.003226 | 0 | 0.002004 | 0.000357 | 0.001463 |
| rf16 | 0.000727 | 0 | 0.003647 | 0.000711 | 0 | 0.000507 | 0.003272 | 0.002137 | 0.004695 | 0.000783 |
| rf17 | 0 | 0.004502 | 0.001319 | 0.003271 | 0.000629 | 0 | 0 | 0 | 0.000347 | 0.003747 |
| rf18 | 6.59E−18 | 0.002015 | 0.002126 | 0.003123 | 0.002087 | 0.002816 | 0.003143 | 0.00339 | 0.004793 | 1.29E−18 |
| rf19 | 4.13E−05 | 0.001565 | 0.00105 | 6.97E−18 | 0.000716 | 0.001092 | 0 | 0.004621 | 0.000701 | 0.001205 |
| rf20 | 0.002622 | 0.002326 | 0.001549 | 0 | 0.004683 | 0 | 0.000854 | 0.004707 | 0.010033 | 0.001018 |
| rf21 | 0.00458 | 0.000861 | 0.000366 | 0.001745 | 0.002508 | 0.001463 | 0.003269 | 0.007573 | 0.001072 | 0.002297 |
| rf22 | 0.004472 | 0.000464 | 2.64E−18 | 3.14E−18 | 0 | 4.15E−18 | 0.001711 | 0.001438 | 0.00647 | 0.001137 |
| rf23 | 6.71E−19 | 0.005028 | 0.001682 | 0.003316 | 0.000789 | 0.002553 | 2.86E−18 | 0.011293 | 0 | 0.003144 |
| rf24 | 0.000697 | 0.003204 | 0.001941 | 0.002455 | 0 | 1.12E−18 | 0 | 0.004544 | 0.003003 | 0 |
| rf25 | 0.000391 | 0.00086 | 0.0039 | 0 | 0.005011 | 7.75E−05 | 0.000928 | 0.000645 | 7.99E−18 | 0.000354 |
| rf26 | 0 | 0.001195 | 0 | 0 | 0 | 0 | 0 | 0.003404 | 0 | 0.000638 |
| rf27 | 0.002493 | 0.001766 | 0 | 0 | 0.003236 | 0.001475 | 0 | 0.00566 | 0.001758 | 0.002722 |
| extraTrees0 | 0.001408 | 0.000856 | 0 | 0.000744 | 0.002493 | 0.001514 | 0.00327 | 0.001082 | 0.003521 | 0.002002 |
| extraTrees1 | 0.001845 | 0.000805 | 0.002241 | 0.002853 | 0.000573 | 0.000655 | 0.000535 | 0.005206 | 0 | 0.000924 |
| extraTrees2 | 0.001292 | 0.001687 | 8.02E−05 | 0.001478 | 0.004394 | 2.20E−18 | 0.000747 | 0.003456 | 0 | 0.000238 |
| extraTrees3 | 0.001201 | 0.002416 | 0.002375 | 0.002432 | 0.000806 | 0.000549 | 0.003743 | 0.001287 | 7.01E−19 | 0.000507 |
| extraTrees4 | 0.000705 | 0.001493 | 0.001444 | 0.000661 | 0.002561 | 0.000512 | 0.001116 | 0.001457 | 8.20E−19 | 0.001477 |
| extraTrees5 | 0.001466 | 0.000844 | 0.001289 | 0.000937 | 0.002806 | 0.001021 | 0.003668 | 0.001122 | 0.001944 | 0.00163 |
| extraTrees6 | 0.000611 | 0 | 0.001207 | 0.001484 | 0.001783 | 0.001518 | 0.000747 | 0.001923 | 1.96E−18 | 0.001216 |
| extraTrees7 | 0.001713 | 0.000254 | 0.001457 | 0.000388 | 0.001097 | 0.000492 | 0.001725 | 0.002512 | 0.001992 | 0.001069 |
| extraTrees8 | 0.000502 | 0.001157 | 0.00045 | 0.00104 | 0.003855 | 0.001199 | 0.00189 | 0.002309 | 0.002317 | 0 |
| extraTrees9 | 0.000847 | 0.000505 | 0.001158 | 0.002299 | 0.003314 | 0.002445 | 0.001424 | 0.003787 | 0.001533 | 0.0005 |
| extraTrees10 | 0.001578 | 0.000388 | 0.000863 | 0.001414 | 0.00263 | 0.000507 | 0.002783 | 0.004855 | 0.005693 | 0.000295 |
| extraTrees11 | 0.001365 | 0.001896 | 0.001682 | 0.000238 | 0.001545 | 0.001279 | 0.001271 | 0.003512 | 0 | 0.001782 |
| extraTrees12 | 0.000654 | 3.07E−18 | 0.001269 | 0.001713 | 0.000586 | 7.56E−05 | 0.000816 | 0.00345 | 0.002026 | 0.002055 |
| extraTrees13 | 0.001118 | 0.002309 | 0.000567 | 0.003523 | 0.00151 | 0.00037 | 0.001879 | 0.001668 | 0 | 0.001206 |
| extraTrees14 | 0.001408 | 0.000856 | 0 | 0.000744 | 0.002493 | 0.001514 | 0.00327 | 0.001082 | 0.003521 | 0.002002 |
| extraTrees15 | 0.001845 | 0.000805 | 0.002241 | 0.002853 | 0.000573 | 0.000655 | 0.000535 | 0.005206 | 0 | 0.000924 |
| extraTrees16 | 0.001292 | 0.001687 | 8.02E−05 | 0.001478 | 0.004394 | 2.20E−18 | 0.000747 | 0.003456 | 0 | 0.000238 |
| extraTrees17 | 0.001201 | 0.002416 | 0.002375 | 0.002432 | 0.000806 | 0.000549 | 0.003743 | 0.001287 | 7.01E−19 | 0.000507 |
| extraTrees18 | 0.000705 | 0.001493 | 0.001444 | 0.000661 | 0.002561 | 0.000512 | 0.001116 | 0.001457 | 8.20E−19 | 0.001477 |
| extraTrees19 | 0.001466 | 0.000844 | 0.001289 | 0.000937 | 0.002806 | 0.001021 | 0.003668 | 0.001122 | 0.001944 | 0.00163 |
| extraTrees20 | 0.000611 | 0 | 0.001207 | 0.001484 | 0.001783 | 0.001518 | 0.000747 | 0.001923 | 1.96E−18 | 0.001216 |
| extraTrees21 | 0.001713 | 0.000254 | 0.001457 | 0.000388 | 0.001097 | 0.000492 | 0.001725 | 0.002512 | 0.001992 | 0.001069 |
| extraTrees22 | 0.000502 | 0.001157 | 0.00045 | 0.00104 | 0.003855 | 0.001199 | 0.00189 | 0.002309 | 0.002317 | 0 |
| extraTrees23 | 0.000847 | 0.000505 | 0.001158 | 0.002299 | 0.003314 | 0.002445 | 0.001424 | 0.003787 | 0.001533 | 0.0005 |
| extraTrees24 | 0.001578 | 0.000388 | 0.000863 | 0.001414 | 0.00263 | 0.000507 | 0.002783 | 0.004855 | 0.005693 | 0.000295 |
| extraTrees25 | 0.001365 | 0.001896 | 0.001682 | 0.000238 | 0.001545 | 0.001279 | 0.001271 | 0.003512 | 0 | 0.001782 |
| extraTrees26 | 0.000654 | 3.07E−18 | 0.001269 | 0.001713 | 0.000586 | 7.56E−05 | 0.000816 | 0.00345 | 0.002026 | 0.002055 |
| extraTrees27 | 0.001118 | 0.002309 | 0.000567 | 0.003523 | 0.00151 | 0.00037 | 0.001879 | 0.001668 | 0 | 0.001206 |
| gbc0 | 0 | 0 | 5.51E−06 | 0.001791 | 0 | 0.000477 | 2.37E−05 | 0 | 0 | 4.62E−05 |
| gbc1 | 0 | 0 | 0 | 0.000178 | 5.82E−05 | 0 | 0 | 0.000251 | 0 | 6.61E−06 |
| gbc2 | 4.68E−05 | 1.29E−06 | 0 | 0.000738 | 0.000499 | 0 | 0 | 0 | 0 | 0 |
| gbc3 | 0.000703 | 0 | 0 | 0.002118 | 3.36E−05 | 0 | 0 | 4.06E−05 | 0.000253 | 0 |
| gbc4 | 0.001868 | 0.001071 | 0.000525 | 0.000789 | 0 | 0.001702 | 0 | 0.00582 | 0.012407 | 0.016831 |
| gbc5 | 0 | 0 | 0.004889 | 0 | 0 | 0 | 0.003192 | 0.004939 | 0.007373 | 1.93E−05 |
| gbc6 | 0 | 0 | 7.47E−06 | 0.001263 | 3.95E−05 | 0.00254 | 2.22E−05 | 0 | 0 | 0 |
| gbc7 | 0 | 8.59E−06 | 0.000625 | 0.01784 | 0 | 6.23E−05 | 0 | 5.72E−05 | 4.00E−05 | 0 |
| gbc8 | 1.49E−05 | 0 | 3.78E−06 | 0 | 0 | 0.004957 | 0 | 0 | 3.51E−06 | 0.00104 |
| gbc9 | 0 | 0.001309 | 3.02E−06 | 0 | 5.60E−05 | 0 | 0 | 0 | 0 | 8.51E−05 |
| gbc10 | 0 | 2.43E−06 | 0 | 0 | 0 | 0 | 0 | 0 | 0.013961 | 0.000925 |
| gbc11 | 0 | 0 | 0 | 0 | 0 | 0 | 0 | 2.76E−06 | 2.55E−05 | 4.78E−07 |
| gbc12 | 4.72E−05 | 0 | 0.000727 | 0 | 0.004945 | 0.000174 | 0 | 0.010551 | 7.41E−05 | 0 |
| gbc13 | 0.001229 | 0 | 0.002924 | 0 | 0 | 0 | 0.00012 | 0.00406 | 0.000975 | 0 |
| gbc14 | 0 | 0 | 5.51E−06 | 0.001791 | 0 | 0.000477 | 2.37E−05 | 0 | 0 | 4.62E−05 |
| gbc15 | 0 | 0 | 0 | 0.000178 | 5.82E−05 | 0 | 0 | 0.000251 | 0 | 6.61E−06 |
| gbc16 | 4.68E−05 | 1.29E−06 | 0 | 0.000738 | 0.000499 | 0 | 0 | 0 | 0 | 0 |
| gbc17 | 0.000703 | 0 | 0 | 0.002118 | 3.36E−05 | 0 | 0 | 4.06E−05 | 0.000253 | 0 |
| gbc18 | 0.001868 | 0.001071 | 0.000525 | 0.000789 | 0 | 0.001702 | 0 | 0.00582 | 0.012407 | 0.016831 |
| gbc19 | 0 | 0 | 0.004889 | 0 | 0 | 0 | 0.003192 | 0.004939 | 0.007373 | 1.93E−05 |
| gbc20 | 0 | 0 | 7.47E−06 | 0.001263 | 3.95E−05 | 0.00254 | 2.22E−05 | 0 | 0 | 0 |
| gbc21 | 0 | 8.59E−06 | 0.000625 | 0.01784 | 0 | 6.23E−05 | 0 | 5.72E−05 | 4.00E−05 | 0 |
| gbc22 | 1.49E−05 | 0 | 3.78E−06 | 0 | 0 | 0.004957 | 0 | 0 | 3.51E−06 | 0.00104 |
| gbc23 | 0 | 0.001309 | 3.02E−06 | 0 | 5.60E−05 | 0 | 0 | 0 | 0 | 8.51E−05 |
| gbc24 | 0 | 2.43E−06 | 0 | 0 | 0 | 0 | 0 | 0 | 0.013961 | 0.000925 |
| gbc25 | 0 | 0 | 0 | 0 | 0 | 0 | 0 | 2.76E−06 | 2.55E−05 | 4.78E−07 |
| gbc26 | 4.72E−05 | 0 | 0.000727 | 0 | 0.004945 | 0.000174 | 0 | 0.010551 | 7.41E−05 | 0 |
| gbc27 | 0.001229 | 0 | 0.002924 | 0 | 0 | 0 | 0.00012 | 0.00406 | 0.000975 | 0 |
| ada0 | 0 | 0 | 0 | 0 | 0 | 0 | 0 | 0 | 0 | 0 |
| ada1 | 0 | 0 | 0 | 0 | 0 | 0 | 0 | 0 | 0 | 0 |
| ada2 | 0 | 0 | 0 | 0 | 0 | 0 | 0 | 0 | 0 | 0 |
| ada3 | 0 | 0 | 0 | 0 | 0 | 0 | 0 | 0 | 0 | 0 |
| ada4 | 0 | 0 | 0 | 0 | 0 | 0 | 0 | 0 | 0 | 0 |
| ada5 | 0 | 0 | 0 | 0 | 0 | 0 | 0 | 0 | 0 | 0 |
| ada6 | 0 | 0 | 0 | 0 | 0 | 0 | 0 | 0 | 0 | 0 |
| ada7 | 0 | 0 | 0 | 0 | 0 | 0 | 0 | 0 | 0 | 0 |
| ada8 | 0 | 0 | 0 | 0 | 0 | 0 | 0 | 0 | 0 | 0 |
| ada9 | 0 | 0 | 0 | 0 | 0 | 0 | 0 | 0 | 0 | 0 |
| ada10 | 0 | 0 | 0 | 0 | 0 | 0 | 0 | 0 | 0 | 0 |
| ada11 | 0 | 0 | 0 | 0 | 0 | 0 | 0 | 0 | 0 | 0 |
| ada12 | 0 | 0 | 0 | 0 | 0 | 0 | 0 | 0 | 0 | 0 |
| ada13 | 0 | 0 | 0 | 0 | 0 | 0 | 0 | 0 | 0 | 0 |
| ada14 | 0 | 0 | 0 | 0 | 0 | 0 | 0 | 0 | 0 | 0 |
| ada15 | 0 | 0 | 0 | 0 | 0 | 0 | 0 | 0 | 0 | 0 |
| ada16 | 0 | 0 | 0 | 0 | 0 | 0 | 0 | 0 | 0 | 0 |
| ada17 | 0 | 0 | 0 | 0 | 0 | 0 | 0 | 0 | 0 | 0 |
| ada18 | 0 | 0 | 0 | 0 | 0 | 0 | 0 | 0 | 0 | 0 |
| ada19 | 0 | 0 | 0 | 0 | 0 | 0 | 0 | 0 | 0 | 0 |
| ada20 | 0 | 0 | 0 | 0 | 0 | 0 | 0 | 0 | 0 | 0 |
| ada21 | 0 | 0 | 0 | 0 | 0 | 0 | 0 | 0 | 0 | 0 |
| ada22 | 0 | 0 | 0 | 0 | 0 | 0 | 0 | 0 | 0 | 0 |
| ada23 | 0 | 0 | 0 | 0 | 0 | 0 | 0 | 0 | 0 | 0 |
| ada24 | 0 | 0 | 0 | 0 | 0 | 0 | 0 | 0 | 0 | 0 |
| ada25 | 0 | 0 | 0 | 0 | 0 | 0 | 0 | 0 | 0 | 0 |
| ada26 | 0 | 0 | 0 | 0 | 0 | 0 | 0 | 0 | 0 | 0 |
| ada27 | 0 | 0 | 0 | 0 | 0 | 0 | 0 | 0 | 0 | 0 |

| Gene | ZNF706 |
|---|---|
| rf0 | 0.001964 |
| rf1 | 0 |
| rf2 | 0.00043 |
| rf3 | 0.002466 |
| rf4 | 8.20E−05 |
| rf5 | 0.012769 |
| rf6 | 0.000323 |
| rf7 | 0 |
| rf8 | 0 |
| rf9 | 0 |
| rf10 | 0 |
| rf11 | 0 |
| rf12 | 8.98E−05 |
| rf13 | 0.002082 |
| rf14 | 0.001964 |
| rf15 | 0 |
| rf16 | 0.00043 |
| rf17 | 0.002466 |
| rf18 | 8.20E−05 |
| rf19 | 0.012769 |
| rf20 | 0.000323 |
| rf21 | 0 |
| rf22 | 0 |
| rf23 | 0 |
| rf24 | 0 |
| rf25 | 0 |
| rf26 | 8.98E−05 |
| rf27 | 0.002082 |
| extraTrees0 | 0.001928 |
| extraTrees1 | 0.000534 |
| extraTrees2 | 0.001691 |
| extraTrees3 | 0.001659 |
| extraTrees4 | 0.000743 |
| extraTrees5 | 0.000714 |
| extraTrees6 | 0.000254 |
| extraTrees7 | 0.001342 |
| extraTrees8 | 0.001399 |
| extraTrees9 | 0.000388 |
| extraTrees10 | 0.00103 |
| extraTrees11 | 0.002503 |
| extraTrees12 | 0.001544 |
| extraTrees13 | 0.002944 |
| extraTrees14 | 0.001928 |
| extraTrees15 | 0.000534 |
| extraTrees16 | 0.001691 |
| extraTrees17 | 0.001659 |
| extraTrees18 | 0.000743 |
| extraTrees19 | 0.000714 |
| extraTrees20 | 0.000254 |
| extraTrees21 | 0.001342 |
| extraTrees22 | 0.001399 |
| extraTrees23 | 0.000388 |
| extraTrees24 | 0.00103 |
| extraTrees25 | 0.002503 |
| extraTrees26 | 0.001544 |
| extraTrees27 | 0.002944 |
| gbc0 | 0 |
| gbc1 | 0 |
| gbc2 | 0 |
| gbc3 | 0 |
| gbc4 | 0 |
| gbc5 | 0 |
| gbc6 | 0 |
| gbc7 | 0 |
| gbc8 | 0 |
| gbc9 | 0 |
| gbc10 | 0 |
| gbc11 | 4.24E−05 |
| gbc12 | 0.001827 |
| gbc13 | 0 |
| gbc14 | 0 |
| gbc15 | 0 |
| gbc16 | 0 |
| gbc17 | 0 |
| gbc18 | 0 |
| gbc19 | 0 |
| gbc20 | 0 |
| gbc21 | 0 |
| gbc22 | 0 |
| gbc23 | 0 |
| gbc24 | 0 |
| gbc25 | 4.24E−05 |
| gbc26 | 0.001827 |
| gbc27 | 0 |
| ada0 | 0 |
| ada1 | 0 |
| ada2 | 0 |
| ada3 | 0 |
| ada4 | 0 |
| ada5 | 0 |
| ada6 | 0 |
| ada7 | 0 |
| ada8 | 0 |
| ada9 | 0 |
| ada10 | 0 |
| ada11 | 0 |
| ada12 | 0 |
| ada13 | 0 |
| ada14 | 0 |
| ada15 | 0 |
| ada16 | 0 |
| ada17 | 0 |
| ada18 | 0 |
| ada19 | 0 |
| ada20 | 0 |
| ada21 | 0 |
| ada22 | 0 |
| ada23 | 0 |
| ada24 | 0 |
| ada25 | 0 |
| ada26 | 0 |
| ada27 | 0 |

### Example 6. PRESS Validation

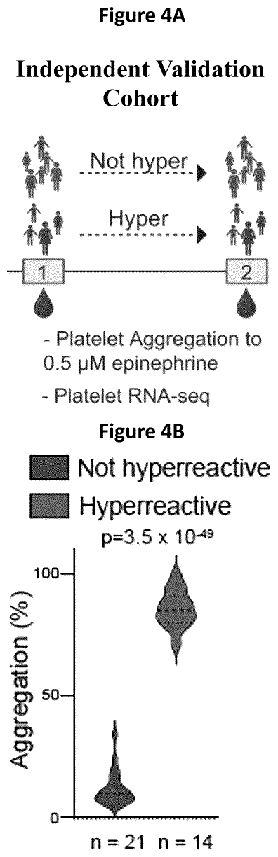

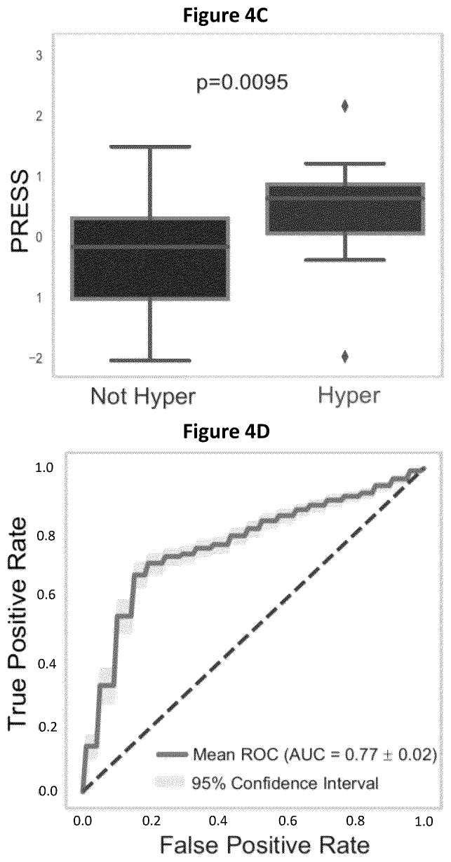

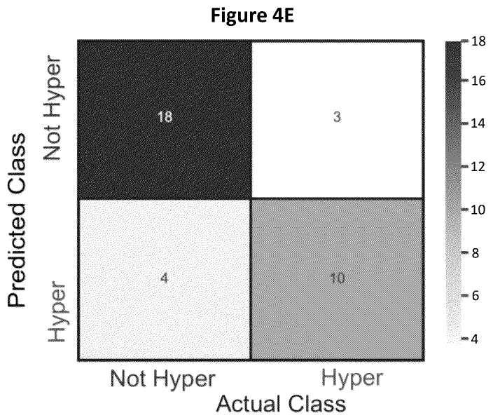

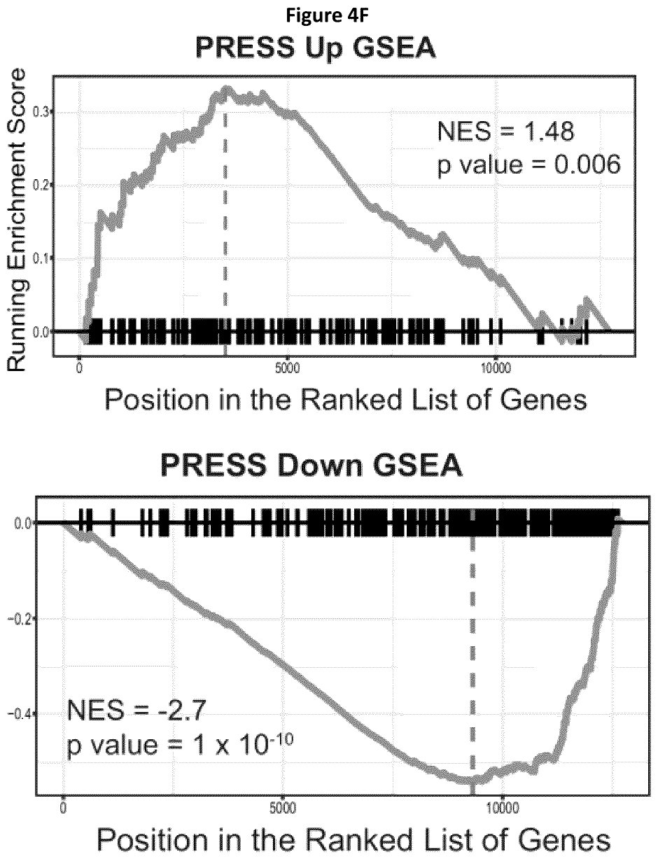

To further evaluate the ability of PRESS to discriminate individuals with hyperreactive platelets, performance was assessed in an independent validation cohort (n=35, free of CVD and off antiplatelet therapy, of which 14 (40%) had a hyperreactive (>60% aggregation) platelet phenotype, FIGS. 4A, 4B; FIG. 6) (32). Within the independent validation cohort, PRESS was significantly higher in those with versus without platelet hyperreactivity (p=0.0095, FIG. 4C). The diagnostic performance of PRESS in the independent validation cohort performed well with an AUC of 0.77 (95% CI 0.75 to 0.79, FIG. 4D). The accuracy was 80%, sensitivity 71%, and specificity 86% when discriminating between those with and without platelet hyperreactivity (FIG. 4E). Furthermore, GSEA pathway analysis, incorporating PRESS in the independent validation cohort, revealed significant enrichment of both upregulated (p=0.006) and downregulated (p<1.0×10⁻¹⁰) PRESS genes in subjects with hyperreactive platelets (FIG. 4F). Collectively, these data demonstrate the utility of PRESS in discriminating platelet hyperreactivity across populations.

### Example 7. PRESS Gene Set is Enriched in Cohorts with Platelet Hyperreactivity

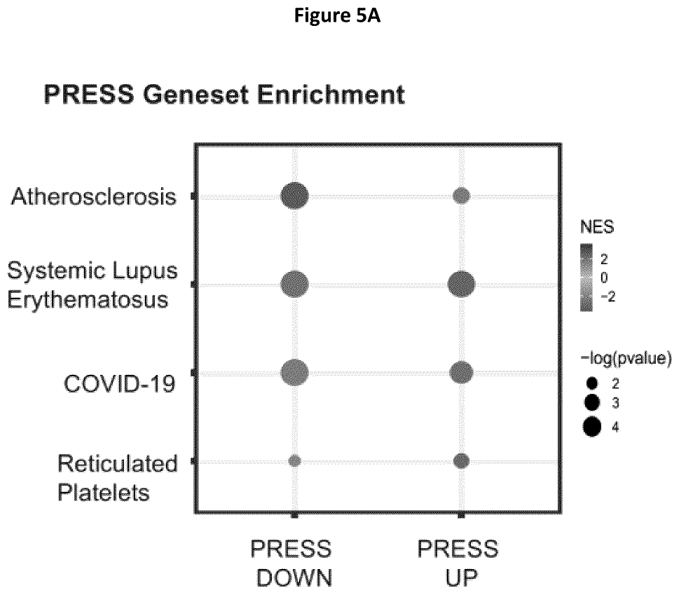

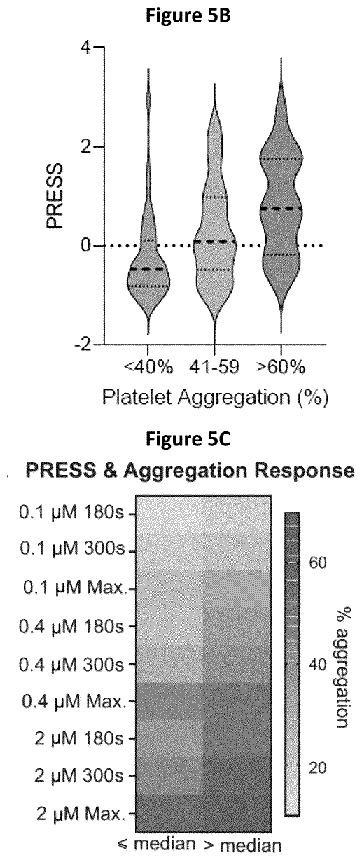

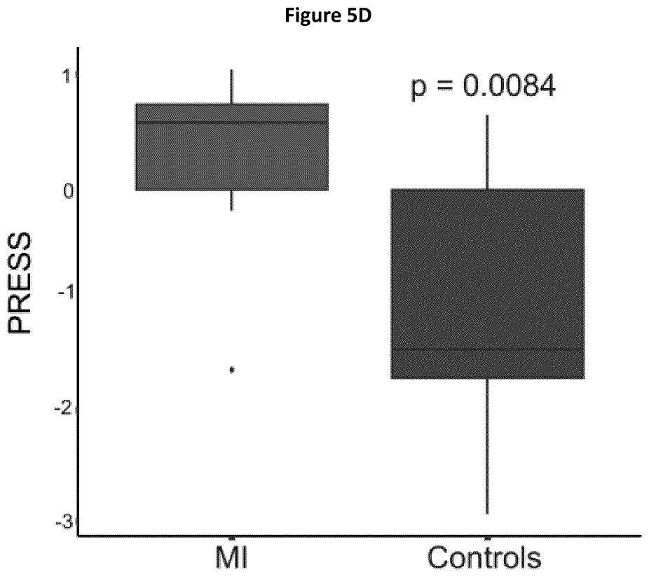

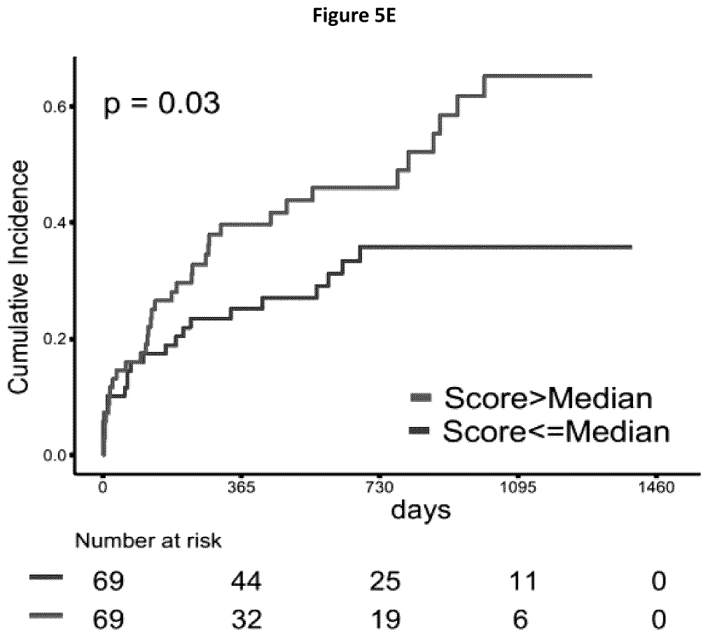

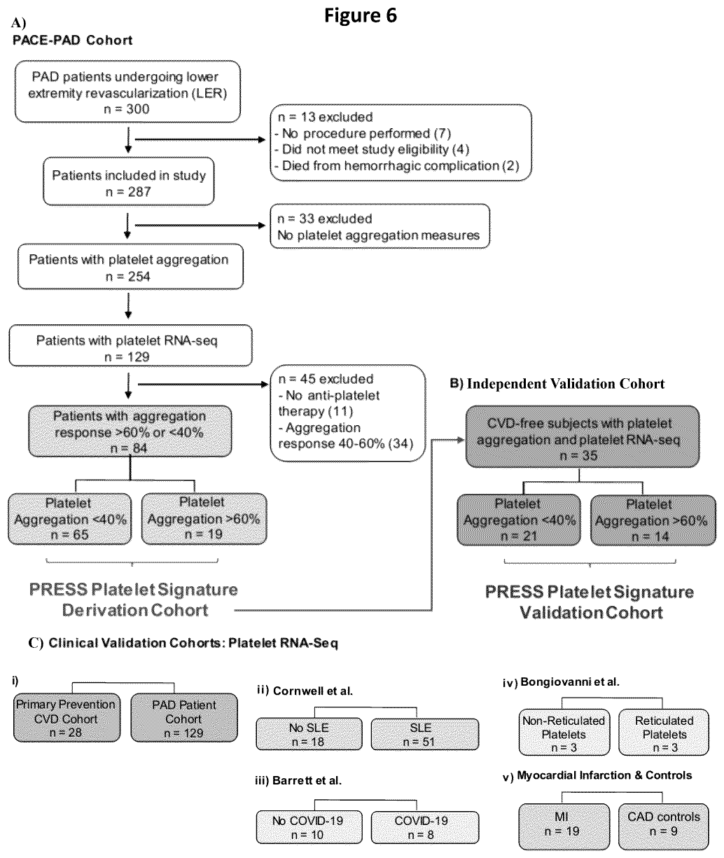

It was reasoned that PRESS could discriminate populations with well-described hyperreactive platelets and a prothrombotic phenotype that underlies CV risk (14). To test this, PRESS was investigated in multiple platelet RNA-seq cohorts from patients with previously described platelet hyperreactivity (FIG. 6) (32-34). First, the platelet transcriptome of patients with established lower extremity atherosclerosis was compared to a high-risk primary prevention cohort (4,35). Incorporating PRESS into the analytical pipeline, PRESS was enriched in the platelet transcriptome of patients with versus without atherosclerosis (PRESS Up and Down, p=0.0007 and p<1×10⁻¹⁰, respectively, FIG. 5A, Table 11). Furthermore, PRESS was significantly enriched in the platelet transcriptome of subjects with both COVID-19 and SLE (FIG. 5, Table 11). Finally, PRESS was enriched in the transcriptome of reticulated (immature) platelets, a hyperreactive and prothrombotic platelet subtype, relative to non-reticulated (mature) platelets (FIG. 5A, Table 11) (36). Collectively, these data demonstrate that PRESS, in lieu of platelet function testing, can discriminate platelet hyperreactivity across disease states.

TABLE 11
PRESS Clinical Validation

| Dataset | geneset | NES | p-value |
|---|---|---|---|
| Atherosclerosis | PRESS UP | 1.60 | 6.53E−04 |
| Atherosclerosis | PRESS DOWN | −3.16 | 1.00E−10 |
| COVID-19 | PRESS UP | 2.09 | 4.16E−08 |
| COVID-19 | PRESS DOWN | −2.09 | 1.00E−10 |
| Systemic Lupus Erythematosus | PRESS UP | 2.33 | 1.00E−10 |
| Systemic Lupus Erythematosus | PRESS DOWN | −2.51 | 1.00E−10 |
| Reticulated Platelets | PRESS UP | 2.27 | 2.72E−04 |
| Reticulated Platelets | PRESS DOWN | −2.37 | 6.79E−04 |

GSEA pathway analysis, incorporating PRESS into multiple clinical cohorts: atherosclerosis (PAD), COVID-19, systemic lupus erythematosus and reticulated platelets with platelet RNA-seq revealed significant enrichment of both upregulated and downregulated PRESS. Refer to FIG. 6 for population descriptions.

### Example 8. PRESS is Associated with Cardiovascular Risk

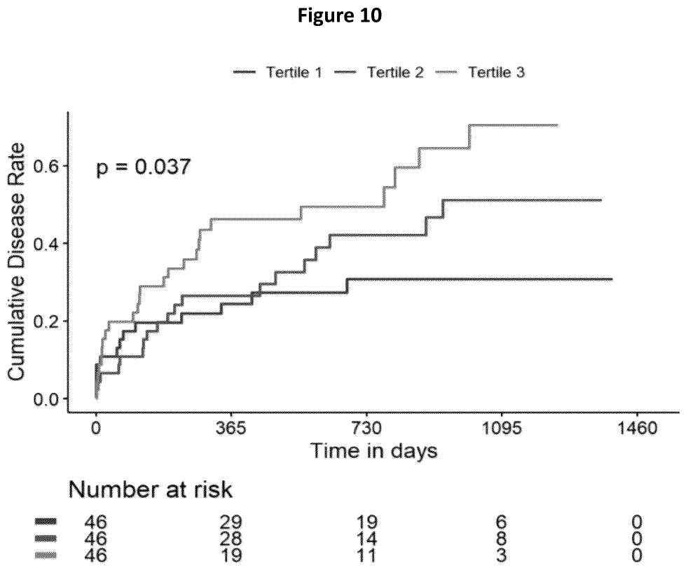

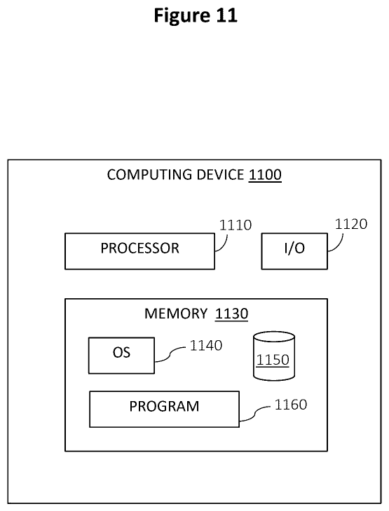

PRESS successfully stratified individuals based on their platelet aggregation response to submaximal agonist stimulation with epinephrine (FIG. 5B), across epinephrine concentrations (FIG. 5C), and across multiple agonists (Table 12). To determine the prognostic value of PRESS, the association between PRESS and CV risk was explored in two populations of CV disease. In the Heart Attack Research Program (HARP), PRESS was calculated in women undergoing coronary angiography. PRESS was significantly increased in women with acute MI compared to women referred for coronary angiography with obstructive coronary artery disease without MI (p-0.0084, FIG. 5D, Table 13). After multivariable adjustment for age, race/ethnicity, diabetes, and hypertension, PRESS was significantly associated with acute MI (B=1.8, 95% CI 0.2 to 3.3. p=0.02). In the second cohort of patients with lower extremity PAD undergoing LER, PRESS was calculated from 129 platelet RNA samples collected prior to LER (Table 14). After a median follow-up of 18 months, the cumulative incidence of major adverse CV events differed by PRESS. After adjusting for age, sex, race/ethnicity, BMI, diabetes, carotid artery disease, prior stroke, and CLI, a PRESS above the median value was associated with a 90% increase in a major CV event (50.7% vs. 31.9% below the median, adjusted HR 1.90, CI 1.07 to 3.36, p=0.027, FIG. 5E). When PRESS was stratified into tertiles, those with a score in the highest tertile were at the highest risk of a major CV event (54.3% vs 28.3% in the lowest tertile, adjusted HR 2.39, CI 1.15 to 4.98, p=0.02. FIG. 10). Altogether, these data demonstrate that PRESS can discriminate those at increased CV risk

TABLE 12
Platelet Aggregation Stratified by Response to 0.4 μM epinephrine

| | No Hyperreactivity | | Hyperreactivity | | |
|---|---|---|---|---|---|
| | ≤60% Aggregation | | >60% Aggregation | | |
| | (n = 203) | | (n = 43) | | P value |
| Epinephrine 2 μM | | | | | |
| 180 s, median (25th, 75th) | 38.00 | [26.00, 50.00] | 79.00 | [74.50, 82.50] | <0.001 |
| 300 s, median (25th, 75th) | 45.00 | [30.50, 58.00] | 84.00 | [81.00, 89.00] | <0.001 |
| max, median (25th, 75th) | 56.50 | [40.45, 70.90] | 88.00 | [85.20, 92.00] | <0.001 |
| Epinephrine 0.4 μM | | | | | |
| 180 s, median (25th, 75th) | 22.00 | [14.00, 33.00] | 62.00 | [53.00, 77.00] | <0.001 |
| 300 s, median (25th, 75th) | 28.00 | [18.00, 40.00] | 80.00 | [69.00, 86.50] | <0.001 |
| max, median (25th, 75th) | 38.30 | [25.35, 52.10] | 86.30 | [79.00, 89.95] | <0.001 |
| Epinephrine 0.1 μM | | | | | |
| 180 s, median (25th, 75th) | 11.00 | [5.00, 19.00] | 32.00 | [21.50, 39.00] | <0.001 |
| 300 s, median (25th, 75th) | 16.00 | [8.00, 24.00] | 47.00 | [36.50, 73.50] | <0.001 |
| max, median (25th, 75th) | 23.50 | [14.40, 34.40] | 81.80 | [59.90, 88.05] | <0.001 |
| ADP 2 μM | | | | | |
| 180 s, median (25th, 75th) | 47.00 | [20.50, 63.50] | 79.00 | [64.50, 82.75] | <0.001 |
| 300 s, median (25th, 75th) | 42.00 | [17.50, 61.00] | 80.50 | [71.00, 87.00] | <0.001 |
| max, median (25th, 75th) | 52.90 | [37.35, 67.25] | 82.75 | [71.83, 88.38] | <0.001 |
| ADP 1 μM | | | | | |
| 180 s, median (25th, 75th) | 18.00 | [3.50, 37.00] | 66.00 | [46.50, 80.00] | <0.001 |
| 300 s, median (25th, 75th) | 18.00 | [6.00, 36.00] | 70.00 | [46.00, 84.50] | <0.001 |
| max, median (25th, 75th) | 32.80 | [17.35, 48.35] | 78.30 | [52.90, 86.30] | <0.001 |
| ADP 0.4 μM | | | | | |
| 180 s, median (25th, 75th) | 3.00 | [0.00, 11.00] | 21.00 | [9.50, 41.50] | <0.001 |
| 300 s, median (25th, 75th) | 6.00 | [0.00, 13.00] | 29.00 | [13.50, 50.50] | <0.001 |
| max, median (25th, 75th) | 13.70 | [6.50, 23.75] | 35.50 | [21.30, 73.20] | <0.001 |
| Collagen 1 μg/ml | | | | | |
| 180 s, median (25th, 75th) | 5.50 | [1.25, 12.00] | 23.00 | [10.50, 62.50] | <0.001 |
| 300 s, median (25th, 75th) | 10.00 | [4.00, 17.00] | 29.00 | [15.00, 74.00] | <0.001 |
| max, median (25th, 75th) | 15.45 | [8.40, 23.17] | 42.80 | [23.95, 77.85] | <0.001 |
| Collagen 0.2 μg/ml | | | | | |
| 180 s, median (25th, 75th) | 3.00 | [0.00, 6.00] | 8.00 | [3.00, 15.00] | <0.001 |
| 300 s, median (25th, 75th) | 6.00 | [1.25, 10.00] | 14.00 | [7.00, 23.00] | <0.001 |
| max, median (25th, 75th) | 11.30 | [7.20, 18.40] | 23.60 | [14.25, 40.30] | <0.001 |
| Serotonin 10 μM | | | | | |
| 180 s, median (25th, 75th) | 0.00 | [0.00, 6.00] | 12.00 | [6.00, 27.00] | <0.001 |
| 300 s, median (25th, 75th) | 2.00 | [0.00, 8.00] | 15.00 | [8.00, 27.50] | <0.001 |
| max, median (25th, 75th) | 11.65 | [6.53, 19.30] | 26.60 | [16.90, 41.00] | <0.001 |
| Serotonin 10 μM + | | | | | |
| Epinephrine 0.1 μM | | | | | |
| 180 s, median (25th, 75th) | 11.00 | [2.25, 22.75] | 67.00 | [35.00, 79.00] | <0.001 |
| 300 s, median (25th, 75th) | 13.50 | [4.00, 25.00] | 80.00 | [45.50, 85.50] | <0.001 |
| max, median (25th, 75th) | 24.60 | [14.48, 36.50] | 83.60 | [62.45, 86.95] | <0.001 |
| Arachidonic Acid 1600 μM | | | | | |
| 180 s, median (25th, 75th) | 18.00 | [9.00, 33.00] | 80.00 | [64.00, 86.00] | <0.001 |
| 300 s, median (25th, 75th) | 21.00 | [9.50, 43.00] | 81.00 | [68.50, 88.00] | <0.001 |
| max, median (25th, 75th) | 27.10 | [13.85, 56.70] | 85.80 | [75.75, 89.15] | <0.001 |
| Arachidonic Acid ex vivo | | | | | |
| 1600 μM | | | | | |
| 180 s, median (25th, 75th) | 9.00 | [2.00, 20.00] | 19.00 | [11.00, 29.25] | <0.001 |
| 300 s, median (25th, 75th) | 10.00 | [3.00, 23.00] | 22.00 | [12.25, 31.75] | <0.001 |
| max, median (25th, 75th) | 16.80 | [7.47, 29.10] | 27.40 | [13.90, 40.95] | 0.001 |

TABLE 13
Demographics and Clinical Characteristics
of the Myocardial Infarction Study Cohort

| | MI | | Controls | | |
|---|---|---|---|---|---|
| | (n = 19) | | (n = 9) | | P value |
| Age, mean (SD) | 60.75 | (13) | 69.1 | (8.1) | 0.59 |
| Female, n (%) | 19 | (100) | 9 | (100) | |
| Race, n (%) | | | | | |
| White | 13 | (68.4) | 5 | (55.6) | 0.52 |
| Black | 4 | (21.1) | 2 | (22.2) | 0.94 |
| Other | 2 | (10.53) | 2 | (22.2) | 0.42 |
| Ethnicity | | | | | |
| Hispanic | 6 | (31.6) | 1 | (11.1) | 0.26 |
| Smoking, n (%) | | | | | |
| Current | 7 | (36.8) | 1 | (11.1) | 0.17 |
| Clinical History, n (%) | | | | | |
| Diabetes | 4 | (21.1) | 5 | (55.6) | 0.07 |
| Hypertension | 10 | (52.6) | 7 | (77.8) | 0.22 |
| Hyperlipidemia | 13 | (68.4) | 6 | (66.7) | 0.93 |

TABLE 14
Baseline characteristics in PACE-PAD subjects with platelet
RNA-Seq and aggregation measures to 0.4 μM epinephrine.

| | Overall | | With RNAseq | | Without RNAseq | | P |
|---|---|---|---|---|---|---|---|
| | (n = 254) | | (n = 129) | | (n = 125) | | value |
| Age, mean (SD) | 70 | (10.26) | 70.7 | (10.37) | 69.3 | (10.14) | 0.288 |
| Female, n (%) | 80 | (31.5) | 45 | (34.9) | 35 | (28.0) | 0.296 |
| Race, n (%) | | | | | | | 0.785 |
| White | 159 | (62.6) | 82 | (63.6) | 77 | (61.6) | |
| Black | 55 | (21.7) | 26 | (20.2) | 29 | (23.2) | |
| Asian | 4 | (1.6) | 3 | (2.3) | 1 | (0.8) | |
| Other | 36 | (14.2) | 18 | (14.0) | 18 | (14.4) | |
| Ethnicity | | | | | | | 1 |
| Hispanic | 49 | (19.4) | 25 | (19.4) | 24 | (19.4) | |
| BMI, mean (SD) | 26.81 | (5.37) | 26.87 | (5.69) | 26.74 | (5.04) | 0.841 |
| Smoking, n (%) | | | | | | | |
| Current | 44 | (17.4) | 19 | (14.7) | 25 | (20.2) | 0.514 |
| Former | 149 | (58.9) | 79 | (61.2) | 70 | (56.5) | |
| Never | 60 | (23.7) | 31 | (24.0) | 29 | (23.4) | |
| Family History of CVD, | 75 | (30.1) | 39 | (30.7) | 36 | (29.5) | 0.946 |
| n (%) | | | | | | | |
| Clinical History, n (%) | | | | | | | |
| CAD | 137 | (53.9) | 68 | (52.7) | 69 | (55.2) | 0.786 |
| Prior MI | 64 | (25.2) | 36 | (27.9) | 28 | (22.4) | 0.386 |
| Prior CVA | 45 | (17.7) | 22 | (17.1) | 23 | (18.4) | 0.907 |
| Carotid artery disease | 39 | (15.4) | 16 | (12.4) | 23 | (18.4) | 0.25 |
| VTE | 35 | (13.8) | 19 | (14.7) | 16 | (12.8) | 0.792 |
| Diabetes | 130 | (51.2) | 70 | (54.3) | 60 | (48.0) | 0.383 |
| COPD | 50 | (19.7) | 23 | (17.8) | 27 | (21.6) | 0.55 |
| Hypertension | 222 | (87.4) | 109 | (84.5) | 113 | (90.4) | 0.219 |
| Hyperlipidemia | 187 | (73.6) | 88 | (68.2) | 99 | (79.2) | 0.065 |
| Cancer | 62 | (24.4) | 29 | (22.5) | 33 | (26.4) | 0.561 |
| Heart Failure | 44 | (17.3) | 20 | (15.5) | 24 | (19.2) | 0.54 |
| Procedure type, n (%) | | | | | | | 0.657 |
| Open | 109 | (42.9) | 59 | (45.7) | 50 | (40.0) | |
| Endovascular | 102 | (40.2) | 51 | (39.5) | 51 | (40.8) | |
| Hybrid | 25 | (9.8) | 12 | (9.3) | 13 | (10.4) | |
| No intervention | 18 | (7.1) | 7 | (5.4) | 11 | (8.8) | |
| Indication, n (%) | | | | | | | |
| Critical Limb Ischemia | 199 | (78.3) | 105 | (81.4) | 94 | (75.2) | 0.296 |
| Rest pain | 105 | (41.3) | 52 | (40.3) | 53 | (42.4) | 0.833 |
| Gangrene | 60 | (23.6) | 35 | (27.1) | 25 | (20.0) | 0.234 |
| Ulceration | 104 | (40.9) | 54 | (41.9) | 50 | (40.0) | 0.862 |
| ABI in index limb, | 0.59 | [0.43, 0.81] | 0.56 | [0.38, 0.78] | 0.60 | [0.47, 0.86] | 0.089 |
| median (IQR) | | | | | | | |
| Baseline Medication, | | | | | | | |
| n (%) | | | | | | | |
| Antiplatelet Therapy | 227 | (89.4) | 118 | (91.5) | 109 | (87.2) | 0.368 |
| Aspirin | 211 | (83.1) | 111 | (86.0) | 100 | (80.0) | 0.264 |
| Clopidogrel | 102 | (40.2) | 49 | (38.0) | 53 | (42.4) | 0.555 |
| Statin | 213 | (83.9) | 106 | (82.2) | 107 | (85.6) | 0.567 |
| Beta Blocker | 154 | (60.6) | 73 | (56.6) | 81 | (64.8) | 0.226 |
| ACE/ARB | 137 | (53.9) | 68 | (52.7) | 69 | (55.2) | 0.786 |
| Baseline Lab Values | | | | | | | |
| Creatinine, mg/dl, median | 1.00 | [0.80, 1.30] | 1.00 | [0.90, 1.30] | 1.00 | [0.80, 1.50] | 0.427 |

(IQR)

ABI = ankle-brachial index; ACE/ARB = angiotensin-converting enzyme/angiotensin receptor blocker; BMI = body mass index; CAD = coronary artery disease; CVA = cerebral vascular accident; CVD = cardiovascular disease; MI = myocardial infarction; VTE = venous thromboembolism; COPD = Chronic obstructive pulmonary disease; IQR = inter-quartile range.

Patients with symptomatic PAD undergoing LER are at high risk for cardiac and limb events, many of which are platelet-mediated. The data herein demonstrate that the platelet phenotype prior to LER is associated with incident cardiac and limb events within the first 30 days post-revascularization. The association between platelet hyperreactivity and 30-day events was independent of demographics, clinical characteristics, perioperative risk, and antiplatelet therapy. Notably, incorporating platelet aggregation into traditional perioperative CV risk models improved both reclassification and risk discrimination.

Clinically, platelet aggregometry is used to assess those with suspected platelet function disorders; however, this technique is labor and time intensive and requires careful quality control and technical expertise in its performance and interpretation. Thus, in conjunction with platelet aggregation measures, a platelet transcriptomic signature that can identify patients with a hyperreactive platelet phenotype was developed and validated herein. This transcriptional signature could discriminate hyperreactive platelet profiles and identify individuals at elevated CV risk. Collectively this study highlights the clinical relevance of platelet hyperreactivity, both at the functional and transcriptomic level.

Platelets are major players in the pathogenesis of atherogenesis and thrombosis (1). In addition to their well-described effects on CV disease, platelets are increasingly recognized for their role in inflammation and immunity (37,38). Increased platelet activity has been observed across multiple disease states (e.g., CV, rheumatological, and infectious) (1,3,5,39,40). Platelet aggregation has previously been proposed as a valuable biomarker of CV risk (41). Spontaneous platelet aggregation measured 3 months following a MI off antiplatelet therapy was associated with CV morbidity and all-cause mortality (3). Subsequent studies noted conflicting and inconsistent results (41). Data from the Framingham study found that in those free of CV disease and without antiplatelet therapy, hyperreactivity to ADP, although not epinephrine, was modestly associated with the risk of MI or stroke during a 20-year follow-up (8). In the current study, prior observations were extended to the perioperative period and demonstrated that platelet hyperreactivity measured before LER is associated with 30 day MACLE. The findings herein are directionally consistent in multiple subgroups, including by age, sex, race/ethnicity, antiplatelet therapy, and CV risk. In fact, platelet hyperreactivity provided additional predictive value beyond traditional assessments of perioperative risk.

Although the data herein suggest a significant association between platelet hyperreactivity and subsequent CV events, routine clinical measurement of platelet aggregation is widely considered infeasible. Variability, poor site standardization, cost, time of processing, and pre- and post-analytical measures (e.g., needle gauge, blood sampling and handling, specimen transport and processing) limit enthusiasm for routinely measuring platelet aggregation (42). Investigation of the platelet transcriptome however increases accessibility and allows unbiased insight into mechanisms and pathways associated with platelet hyperreactivity.

The platelet transcriptomic signature disclosed herein is able to differentiate individuals with platelet hyperreactivity. While PRESS was developed in patients with symptomatic PAD on antiplatelet therapy, discriminating platelet hyperreactivity in patients on and off antiplatelet therapy is clinically appealing and removes a major barrier to platelet function testing. Thus, the validation of the signature in an external cohort of healthy subjects who were not taking any antiplatelet therapy points to the robustness and utility of this signature. In fact, the PRESS gene set contains several transcripts linked to platelet activation responses (e.g. P2RXI (43), RHOA (44), WDRI (15), CD69 (45)), and is characterized by pathways associated with platelet activation (anchoring junctions, organelle organization, cytoskeleton reorganization, and vesicle-mediated transport). Altogether, these data demonstrate that individuals with hyperreactive platelets have a unique transcriptome reflective of a primed activation state.

Since PRESS successfully discriminated platelet hyperreactivity on and off antiplatelet therapy, it was investigated if this signature was enriched in platelets of patients with diseases known to have a hyperreactive platelet phenotype. Consistently, PRESS was among the top candidate pathways enriched in patients with well-documented platelet hyperreactivity, including lower extremity atherosclerosis, COVID-19, and SLE. Additionally, as further validation, PRESS was incorporated into GSEA analyses of platelet RNA-seq of reticulated versus non-reticulated platelets. Since reticulated platelets are larger, younger, and immature platelets with increased activity and greater prothrombotic potential, PRESS was found to be amongst the most enriched pathways in this platelet population.

Several factors were considered when interpreting the results of this study. First, the population that derived the platelet hyperreactivity signature had symptomatic PAD on antiplatelet therapy. Nonetheless, the PRESS gene signature was validated in subjects without clinical CV disease, highlighting the signature's ability to discriminate platelet hyperreactivity in subjects with and without CV disease and on- and off-platelet therapy. In the subgroup of patients used for the PRESS derivation cohort (n=84), participants with platelet hyperreactivity trended towards more frequent cardiovascular events. However, in this smaller cohort statistical significance was not reached (Table 15). Second, while submaximal epinephrine was used to define platelet hyperreactivity, the data herein are consistent across various agonists and concentrations (Table 12). Additionally, submaximal epinephrine was chosen as previous studies have shown a distinct bimodal platelet aggregation response reproducible over time and generalizable to other forms of platelet activation (9,10,47). Third, platelet activity was measured at the time of LER in the patient population herein, a procedure that is performed throughout the day. Thus platelet activity measures may be affected by circadian variation. Finally, platelet hyperreactivity was associated with 30-day CV and limb events.

TABLE 15
Platelet activity and adverse outcomes in the RNA-seq derivation cohort.

| | Platelet | No Platelet | | |
|---|---|---|---|---|
| Adverse Events, | Hyperreactivity | Hyperreactivity | Unadjusted | |
| n (%) | (n = 19) | (n = 65) | HR [95% CI] | p-value |

30-day adverse
event, n (%)

| MACLE | 5 | (26.3) | 11 | (16.9) | 1.58 | [0.79, 3.16] | 0.201 |
|---|---|---|---|---|---|---|---|
| MI | 2 | (10.5) | 3 | (4.6) | 2.49 | [0.42, 14.92] | 0.317 |
| MACE | 3 | (15.8) | 3 | (4.6) | 3.47 | [0.70, 17.19] | 0.128 |
| MALE | 2 | (10.5) | 5 | (7.7) | 1.40 | [0.27, 7.23] | 0.686 |
| Any adverse | | | | | | | |
| event n (%) | | | | | | | |
| MACLE | 11 | (57.9) | 29 | (44.6) | 1.58 | [0.79, 3.16] | 0.201 |
| MI | 4 | (21.1) | 5 | (7.7) | 3.31 | [0.88, 12.47] | 0.077 |
| MACE | 5 | (26.3) | 11 | (16.9) | 2.10 | [0.72, 6.09] | 0.174 |
| MALE | 8 | (42.1) | 17 | (26.2) | 1.73 | [0.75, 4.02] | 0.200 |

MACLE, major adverse cardiac and limb events, defined as a composite of death, myocardial infarction (MI), stroke, major amputation, or acute limb ischemia leading to revascularization; MACE, major adverse cardiac events, defined as a composite of death, MI, stroke; MALE, major adverse limb events, defined as a composite of major amputation or acute limb ischemia leading to revascularization. Frequencies of adverse events between groups were compared using Chi-squared test or fisher's exact test. Unadjusted and adjusted Hazard ratios and 95% CIs were computed by cox proportional hazard models.

In individuals with symptomatic PAD undergoing LER, platelet aggregation in response to submaximal epinephrine is associated with 30-day MACLE. A platelet transcriptional signature, PRESS, can discriminate individuals with a hyperreactive platelet phenotype and identify those at increased risk for CV events. This platelet hyperreactivity score can be leveraged to investigate platelet-mediated diseases and personalized diagnostics to improve CV risk reduction.

### Example 9. PRESS Response to Antiplatelet Therapy

Identifying platelet hyperreactivity is critical for selecting patients who may benefit from antiplatelet therapy in cardiovascular (CV) prevention. In this Example, the effect of antiplatelet therapy on PRESS was studied. In an open-label, randomized crossover study, participants without established CV disease received aspirin (81 mg vs. 325 mg daily) and ticagrelor (90 mg twice daily) for 4 weeks each. Platelet activity was assessed, PRESS was calculated, and self-reported bleeding events were recorded before and after each treatment phase. The primary endpoint was PRESS, with a key secondary endpoint of a composite platelet function score (PFS). Among 63 participants (mean age 44 years; 67% female; 54% non-white) with platelet RNA sequencing data, 25 (39.7%) exhibited high PRESS (>0.37), consistent with a platelet hyperreactive phenotype. High PRESS participants demonstrated significantly greater platelet aggregation in response to submaximal epinephrine (p=0.003) and collagen (p=0.036), and higher composite PFS (p=0.028). Aspirin (both 81 mg and 325 mg) and ticagrelor each significantly reduced PFS (p<0.001 for all). Aspirin had no significant effect on PRESS, whereas ticagrelor induced a significant reduction in PRESS (p<0.01). Stratified analyses showed ticagrelor significantly decreased PRESS in participants with high baseline PRESS (p<0.0001), but not in those with normal baseline scores (p=0.69). Minor bleeding was reported in five participants following ticagrelor administration; these individuals showed a non-significant trend toward lower PRESS both pre-(p=0.108) and post-ticagrelor (p=0.088). This Example shows that PRESS is a modifiable biomarker responsive to ticagrelor therapy and can serve as a useful tool to identify individuals who derive optimal risk-benefit balance from primary prevention with antiplatelet agents.

Below are the methods used in the Examples described above.

### Study Cohort

All participants provided written informed consent. Male and female patients (n=300)>21 years of age with PAD undergoing non-emergent LER were recruited into the PACE-PAD study (NCT02106429). Individuals were excluded if they used NSAIDs in the past 72 hours, had a platelet count <100×10⁹/L or >500×10⁹/L, hemoglobin <8 mg/dl, or any known hemorrhagic diathesis.

### Monitoring of Post-Procedure Cardiovascular Events

Patients were followed for 30 days following LER to assess post-operative CV events (4,35). Surveillance electrocardiograms (ECG) and cardiac troponin I concentrations were measured on post-procedural day 2. Blood collections and ECGs performed as part of routine care were used to ascertain event occurrence after postoperative day 2. After hospital discharge, patients were contacted by telephone (or during their routine clinical follow-up) to identify additional events, quality of life, and medication use. The primary endpoint was a composite of major adverse cardiac or limb events (MACLE), defined by major adverse cardiovascular events (MACE; death, myocardial infarction (MI), stroke) and major adverse limb events (MALE; major amputation, or major [new bypass graft, jump/interposition graft revisions or thrombectomy/thrombolysis] or minor [balloon angioplasty, atherectomy, laser treatment and/or stenting or stent/grafting] reintervention of the index limb) at 30 days. Two cardiologists and one vascular surgeon blinded to the platelet aggregation results adjudicated all events by review of medical records. Additional details regarding endpoints are provided in the methods described herein.

### Blood Collection

Before LER, peripheral blood samples were collected from fasting patients at the onset of the procedure. After an initial 2 cc discard, whole blood was collected into tubes containing 3.2% sodium citrate (BD Vacutainer, cat #369714) for platelet activity measures and collection of platelets for subsequent RNA isolation. Blood specimens were immediately processed, and citrate-anticoagulated blood was centrifuged (within 30 minutes of collection) at 200 g for 10 min to obtain platelet-rich plasma (48).

### Platelet Aggregation

Light Transmission Aggregometry (LTA) measures the kinetics of platelet aggregation in a glycoprotein IIb/IIIa-dependent manner and is the most widely used measurement of platelet function (49,50). Platelet aggregometry was conducted on a Helena (Beaumont, TX) AggRAM light transmission aggregometer based on the method of Born (51). Platelet aggregation was measured in response to submaximal agonist stimulation. Agonists included epinephrine (2, 0.4, 0.1 μM, Helena Laboratories, cat #5367), adenosine diphosphate (ADP; 2, 1, 0.4 μM, Helena Laboratories, cat #5366), collagen (1, 0.2 μg/ml, Helena Laboratories, cat #5368), serotonin (10 μM, Sigma Aldrich, cat #H9523), epinephrine and serotonin (1 and 10 μM respectively) and arachidonic acid (AA; 1.6 mM, Helena Laboratories, cat #5364) incubated with and without aspirin in vitro (3 mM, Santa Cruz Biotechnology, cat #sc-202471). Platelet hyperreactivity was defined by platelet aggregation >60% in response to submaximal epinephrine 0.4 μM (10).

### Platelet Transcriptome

Blood was collected for platelet RNA sequencing at the same time as measures of platelet aggregation. Briefly, platelets were isolated and purified by incubation with microbeads to deplete leukocytes and red blood cells (EasySep™ Human CD45 Depletion Kit, StemCell Technologies, cat #17898 and EasySep™ RBC Depletion Reagent, StemCell Technologies, cat #18170). Isolated platelets lysed in 500 μL of QIAzol Lysis Reagent (Qiagen, cat #79306) and stored at −80° C. RNA was isolated with Direct-zol RNA microspin columns (Zymo Research, cat #R2062), and RNA quality and quantity were determined with a Bioanalyzer 2100 (Agilent Technologies). Sequencing libraries were barcoded and prepared using the Clontech SMART-Seq HT with Nxt HT kit (Takara Bio USA, cat #634437), and libraries were sequenced single end on an Illumina NovaSeq 6000 (15,32,39,52). Samples were analyzed using the Seq-N-Slide pipeline (53). Reads were aligned to the hg38 genome using STAR (54) v2.6.1 and quantified using featureCounts (55) v1.6.3. Read quality was assessed using FASTQC (56) v0.11.7.

### Development and Application of the Platelet Reactivity Expression Score (PRESS)

The platelet transcriptome was compared between patients with a hyperreactive phenotype (>60% aggregation in response to submaximal epinephrine, 0.4 μM) relative to those with a platelet aggregation response <40% in response to submaximal epinephrine (0.4 μM) (10). To develop PRESS, genes differentially expressed between groups were identified using the DESeq2 Wald test (57) and the more conservative Wilcoxon rank-sum test after adjustment for age, sex, and race/ethnicity. Only transcripts significantly different (p<0.05) in both tests were retained for downstream analysis. A nominal p-value cutoff of 0.05 was used as very few genes reached significance after the correction of Benjamini-Hochberg multiple testing. To increase confidence in the final gene set, transcripts significantly correlated with the percent of platelet aggregation in response to submaximal agonist stimulation were identified with epinephrine 0.4 μM as a continuous variable. The intersect of genes identified as significantly differential using both the DESeq2 Wald test, and the Wilcoxon rank-sum and significantly correlated with platelet aggregation were then used to generate the Platelet Reactivity Expression Score (PRESS).

To assign PRESS at a sample level, a soft voting ensemble model of random forests, extra-trees forests, adaboost forests, and gradient boosted forests (n=28 each) was built. Internal classifiers of the soft voting ensemble model were tuned to optimize area under the receiver operating curve (AUC) using GridSearchCV. Each model was trained on the scaled and normalized transcript expression of PRESS genes. To assess model training performance, the mean and 95% confidence interval (CI) of the AUC was calculated with 10-fold cross-validation. After cross-validation, the model was refit on all samples for downstream testing and validation in external datasets. The model was used to generate a PRESS score for each individual, presented as a z-scored predictive probability, which was used to represent sample-specific platelet reactivity.

### Platelet Aggregation Validation Cohort

The PRESS model was validated in an external cohort of healthy participants who enrolled in a clinical study examining the effect of antiplatelet therapy on platelet activity and the platelet transcriptome (58). Data was used from patients without antiplatelet therapy for >4 weeks before blood collection. Platelet LTA was measured in response to submaximal epinephrine 0.5 μM, and platelet RNA was isolated and sequenced (58). Consistent with the derivation cohort in patients with PAD described herein, PRESS was compared between patients with a hyperreactive platelet phenotype (>60% aggregation) and those with aggregation <40% in response to submaximal epinephrine stimulation. Performance of the PRESS model was validated within this independent validation cohort with 1000-fold bootstrapping to determine AUC and 95% confidence intervals for external model performance.

### Platelet Hyperreactivity PRESS Gene Set

The PRESS gene set was divided into upregulated (PRESS-up) and downregulated (PRESS-down) genes. To reduce potential bias in the validation of PRESS by GSEA, the gene set was reduced only to include genes that are part of the GO term universe of genes (164/174 for PRESS-up; 257/277 for PRESS-down). Pre-ranked gene set enrichment analysis (GSEA) was performed using the log 2 fold change (log 2fc) values as a ranking method (59,60). All GSEA were performed using ClusterProfiler (61).

### Clinical Validation Cohorts (FIG. 6)

Platelet RNA collected and sequenced were used for validation of the PRESS in multiple cohorts: (1) Patients with lower extremity atherosclerosis (n=129) versus a high-risk primary prevention cohort (n=28, multiple CV risk factors without clinical atherosclerosis) (35 39); (2) systemic lupus erythematosus (SLE, n=51) versus controls (n=18); (3) COVID-19 (n=8) versus controls (n=10) (34); (4) previously published transcriptome analysis of reticulated (immature) versus non-reticulated (mature) platelets (36); and (5) subjects recruited into the Women's Heart Attack Research Program (HARP, NCT03022552) who presented with acute MI (n=19) and obstructive coronary artery disease (n=9) confirmed by coronary angiography. Following adjustment for age, sex, and race/ethnicity, PRESS was compared between groups. For all studies, with the exception of HARP, both men and women were included. HARP only contains women. Sex was determined by self-report during study admission and consent.

### Statistical Analysis

When comparing participant demographics and clinical variables between groups, 2-sample t-tests or Wilcoxon tests for continuous variables and ×2 tests or Fisher's exact tests for categorical variables were used. Categorical variables are presented as numbers (percentage) and continuous variables as mean+SD or median (interquartile range) as appropriate. To investigate the association between platelet hyperreactivity and MACLE up to 30 days post-procedure, patients were divided into two groups based on aggregation responses to submaximal epinephrine (0.4 μM): hyperreactive (>60% aggregation) and not hyperreactive (≤60% aggregation) (9,10,47). Cox proportional hazard regressions were used to assess the relationship between platelet hyperreactivity and 30-day MACLE. Models were adjusted for age, sex, race/ethnicity, and population-relevant clinical risk factors, including BMI, diabetes, carotid artery disease, prior stroke, and critical limb ischemia (CLI). In the HARP cohort, which includes only females, sex was not included in multivariable models. In PACE, CLI (the most severe form of PAD) and carotid artery disease were included in the multivariable model. These two variables were not available in the non-PAD cohorts.

Survival curves were plotted using the Kaplan-Meier method, and differences between groups were determined using log-rank tests. Analyses were repeated after excluding patients who did not receive antiplatelet therapy at baseline blood collection. Subgroup analyses were performed stratified by age, sex, race/ethnicity, body mass index, diabetes mellitus, coronary artery disease, medical therapy, and clinical presentation.

A Cox proportional hazard regression model was generated with the Revised Cardiac Risk Index (RCRI) as a covariate to predict MACLE at 30 days to assess whether platelet aggregation measures improve perioperative CV risk prediction (27). Platelet hyperreactivity was subsequently added to the model. Harrell's C-statistics and 95% CI were computed for model discrimination; a z-score test was constructed to compare the two C statistics (62). Also assessed were the net reclassification improvement (NRI; the probability that patients are reclassified to higher or lower risk) and integrated discrimination improvement (IDI; to quantify mean predicted probabilities of events and nonevents based on the addition of the new biomarkers to the model) (63).

In the cohort of PACE-PAD with RNA sequencing data available, each participant was assigned a hyperreactivity score (PRESS) using the predicted probability of the ensemble model. During a median follow-up of 18 months, it was investigated whether PRESS was associated with a composite of death, MI, stroke, and major amputation. Cumulative event rates were estimated using Kaplan-Meier curves stratified by groups and were compared using the log-rank test. Cox proportional hazards regression analyses were performed to investigate the association of PRESS with outcomes in both univariable and multivariable analyses adjusting for demographics and CV risk factors, including age, sex, race/ethnicity, BMI, diabetes, carotid artery disease, prior stroke, and critical limb ischemia.

Statistical analysis was performed with Rstudio, version 4.0.3. A 2-sided value of P<0.05 was considered statistically significant. Machine learning training and validation were performed in Python, version 3.10.6.

### Data & Code Availability

The platelet RNA-seq data is deposited in the GEO database under accession code GSE232027. Additional data is on the GEO database under the accession codes GSE226147, GSE126448, and GSE176480. Code is available on github.com/ruggleslab/press (10.5281/zenodo.12537341).

### Endpoint Definitions

### Myocardial Infarction

The myocardial infarction (MI) definition follows the universal criteria for MI events developed by the joint ESC/ACCF/AHA/WHF task force (65). Myocardial infarction is defined as a rise and/or fall of cardiac biomarker values (preferably troponin) with at least one value above the 99th percentile upper reference limit (URL) and with at least one of the following: clinical symptoms consistent with myocardial ischemia, development of new pathological Q waves in at least 2 contiguous leads on the electrocardiogram (ECG), ECG changes indicative of new ischemia (new ST-T changes or new left bundle branch block), imaging evidence of new loss of viable myocardium or new regional wall motion abnormality or identification of intracoronary thrombus by angiography or autopsy.

### Stroke Transient Ischemic Attack (TIA)

The stroke/TIA definition follows the criteria set forth by the American Heart Association and American Stroke Association (66). Stroke is defined as a new, sudden, focal neurological deficit resulting from a presumed cerebrovascular cause that is not reversible within 24 hours and not due to a readily identifiable cause such as a tumor or seizure. If an event matching this definition lasts less than 24 hours it will be considered a TIA. However, if a patient has a clinical event that lasts less than 24 hours, but has imaging evidence of acute infarction, then it will be considered a stroke as opposed to a TIA (67). A TIA is a transient episode of neurological dysfunction caused by focal brain, spinal cord, or retinal ischemia, without acute infarction. All suspected strokes (including TIA) will be reviewed and adjudicated by the adjudicating committee.

### Major Amputation

Major amputation was defined as an above ankle amputation, consisting of hip disarticulation, above-knee (transfemoral), or below-knee (transtibial) amputation (68).

### Acute Limb Ischemia

The acute limb ischemia definition required a clinical presentation consistent with acute ischemia (symptoms consistent with a rapid or sudden decrease in limb perfusion including findings on physical examination or imaging requiring an urgent major (New bypass graft, jump/interposition graft revision, thrombectomy) or minor (angioplasty, atherectomy, laser treatment, stent placement, or stent-graft placement) revascularization) (69,70).

### REFERENCES

1 Davi, G. & Patrono, C. Platelet activation and atherothrombosis. N Engl J Med 357, 2482-2494 (2007).
2 Falk, E. Coronary thrombosis: pathogenesis and clinical manifestations. Am J Cardiol 68, 28B-35B (1991).
3 Trip, M. D., Cats, V. M., van Capelle, F. J. & Vreeken, J. Platelet hyperreactivity and prognosis in survivors of myocardial infarction. N Engl J Med 322, 1549-1554 (1990).
4 Dann, R. et al. Platelet-Derived MRP-14 Induces Monocyte Activation in Patients With Symptomatic Peripheral Artery Disease. J Am Coll Cardiol 71, 53-65 (2018).
5 Rajagopalan, S. et al. Platelet activation increases with the severity of peripheral arterial disease: implications for clinical management. J Vasc Surg 46, 485-490 (2007).
6 Gurbel, P. A., Becker, R. C., Mann, K. G., Steinhubl, S. R. & Michelson, A. D. Platelet function monitoring in patients with coronary artery disease. J Am Coll Cardiol 50, 1822-1834 (2007).
7 Michelson, A. D. Methods for the measurement of platelet function. Am J Cardiol 103, 20A-26A (2009).
8 Puurunen, M. K. et al. ADP Platelet Hyperreactivity Predicts Cardiovascular Disease in the FHS (Framingham Heart Study). J Am Heart Assoc 7 (2018).
9 Berger, J. S. et al. Hyperreactive Platelet Phenotypes: Relationship to Altered Serotonin Transporter Number, Transport Kinetics and Intrinsic Response to Adrenergic Co-Stimulation. Thrombosis and Haemostasis 109, 85-92 (2012).
10 Yee, D. L., Sun, C. W., Bergeron, A. L., Dong, J. F. & Bray, P. F. Aggregometry detects platelet hyperreactivity in healthy individuals. Blood 106, 2723-2729 (2005).
11 Breddin, H. K. et al. Spontaneous platelet aggregation as a predictive risk factor for vascular occlusions in healthy volunteers? Results of the HAPARG Study. Haemostatic parameters as risk factors in healthy volunteers. Atherosclerosis 144, 211-219 (1999).
12 Elwood, P. C., Renaud, S., Beswick, A. D., O'Brien, J. R. & Sweetnam, P. M. Platelet aggregation and incident ischaemic heart disease in the Caerphilly cohort. Heart 80, 578-582 (1998).
13 Cattaneo, M. et al. Results of a worldwide survey on the assessment of platelet function by light transmission aggregometry: a report from the platelet physiology subcommittee of the SSC of the ISTH. J Thromb Haemost 7, 1029 (2009).
14 Davizon-Castillo, P., Rowley, J. W. & Rondina, M. T. Megakaryocyte and Platelet Transcriptomics for Discoveries in Human Health and Disease. Arterioscler Thromb Vasc Biol 40, 1432-1440 (2020).
15 Montenont, E. et al. Platelet WDR 1 suppresses platelet activity and is associated with cardiovascular disease. Blood 128, 2033-2042 (2016).
16 Edelstein, L. C. et al. Racial differences in human platelet PAR4 reactivity reflect expression of PCTP and miR-376c. Nat Med 19, 1609-1616 (2013).
17 Eicher, J. D. et al. Characterization of the platelet transcriptome by RNA sequencing in patients with acute myocardial infarction. Platelets 27, 230-239 (2016).
18 In ‘t Veld, S. et al. Detection and localization of early- and late-stage cancers using platelet RNA. Cancer Cell 40, 999-1009 e1006 (2022).
19 Best, M. G., In ‘t Veld, S., Sol, N. & Wurdinger, T. RNA sequencing and swarm intelligence-enhanced classification algorithm development for blood-based disease diagnostics using spliced blood platelet RNA. Nat Protoc 14, 1206-1234 (2019).
20 Fujita, B. et al. Predictive value of gene expression profiling for long-term survival after heart transplantation. Transpl Immunol 41, 27-31 (2017).
21 Creager, H. M. et al. Clinical evaluation of the BioFire (R) Respiratory Panel 2.1 and detection of SARS-COV-2. J Clin Virol 129, 104538 (2020).
22 Yu, W. et al. Exosome-based liquid biopsies in cancer: opportunities and challenges. Ann Oncol 32, 466-477 (2021).
23 Criqui, M. H. et al. Lower Extremity Peripheral Artery Disease: Contemporary Epidemiology, Management Gaps, and Future Directions: A Scientific Statement From the American Heart Association. Circulation 144, e171-e191 (2021).
24 Cassar, K., Bachoo, P., Ford, I., Greaves, M. & Brittenden, J. Platelet activation is increased in peripheral arterial disease. J Vasc Surg 38, 99-103 (2003).
25 Allen, N. et al. Circulating monocyte-platelet aggregates are a robust marker of platelet activity in cardiovascular disease. Atherosclerosis 282, 11-18 (2019).
26 Golomb, B. A., Dang, T. T. & Criqui, M. H. Peripheral arterial disease: morbidity and mortality implications. Circulation 114, 688-699 (2006).
27 Lee, T. H. et al. Derivation and prospective validation of a simple index for prediction of cardiac risk of major noncardiac surgery. Circulation 100, 1043-1049 (1999).
28 Schubert, S., Weyrich, A. S. & Rowley, J. W. A tour through the transcriptional landscape of platelets. Blood 124, 493-502 (2014).
29 Cofer, L. B., Barrett, T. J. & Berger, J. S. Aspirin for the Primary Prevention of Cardiovascular Disease: Time for a Platelet-Guided Approach. Arterioscler Thromb Vasc Biol 42, 1207-1216 (2022).
30 Healy, A. M. et al. Platelet expression profiling and clinical validation of myeloid-related protein-14 as a novel determinant of cardiovascular events. Circulation 113, 2278-2284 (2006).
31 Rondina, M. T. et al. Longitudinal RNA-Seq Analysis of the Repeatability of Gene Expression and Splicing in Human Platelets Identifies a Platelet SELP Splice QTL. Circ Res 126, 501-516 (2020).
32 Barrett, T. J. et al. Platelets amplify endotheliopathy in COVID-19. Sci Adv 7, eabh2434 (2021).
33 Manne, B. K. et al. Platelet gene expression and function in patients with COVID-19. Blood 136, 1317-1329 (2020).
34 El Bannoudi, H. et al. Platelet LGALS3BP Induces Myeloid Inflammation In Systemic Lupus Erythematosus. Arthritis Rheumatol (2022).
35 Newman, J. D. et al. Gene Expression Signature in Patients With Symptomatic Peripheral Artery Disease. Arterioscler Thromb Vasc Biol 41, 1521-1533 (2021).
36 Bongiovanni, D. et al. Transcriptome Analysis of Reticulated Platelets Reveals a Prothrombotic Profile. Thromb Haemost 119, 1795-1806 (2019).
37 Gawaz, M., Langer, H. & May, A. E. Platelets in inflammation and atherogenesis. J Clin Invest 115, 3378-3384 (2005).
38 Koupenova, M., Clancy, L., Corkrey, H. A. & Freedman, J. E. Circulating Platelets as Mediators of Immunity, Inflammation, and Thrombosis. Circ Res 122, 337-351 (2018).
39 Barrett, T. J. et al. Platelets contribute to disease severity in COVID-19. J Thromb Haemost 19, 3139-3153 (2021).
40 Steinhubl, S. R. & Moliterno, D. J. The role of the platelet in the pathogenesis of atherothrombosis. Am J Cardiovasc Drugs 5, 399-408 (2005).
41 Sharma, G. & Berger, J. S. Platelet activity and cardiovascular risk in apparently healthy individuals: a review of the data. J Thromb Thrombolysis 32, 201-208 (2011).
42 Gorog, D. A. & Fuster, V. Platelet function tests in clinical cardiology: unfulfilled expectations. J Am Coll Cardiol 61, 2115-2129 (2013).
43 Mahaut-Smith, M. P., Tolhurst, G. & Evans, R. J. Emerging roles for P2X1 receptors in platelet activation. Platelets 15, 131-144 (2004).
44 Aslan, J. E. & McCarty, O. J. Rho GTPases in platelet function. J Thromb Haemost 11, 35-46 (2013).
45 Testi, R., Pulcinelli, F., Frati, L., Gazzaniga, P. P. & Santoni, A. CD69 is expressed on platelets and mediates platelet activation and aggregation. J Exp Med 172, 701-707 (1990).
46 Berger, J. S. Aspirin for Primary Prevention-Time to Rethink Our Approach. JAMA Netw Open 5, e2210144 (2022).
47 Yee, D. L., Bergeron, A. L., Sun, C. W., Dong, J. F. & Bray, P. F. Platelet hyperreactivity generalizes to multiple forms of stimulation. J Thromb Haemost 4, 2043-2050 (2006).
48 Merolla, M., Nardi, M. A. & Berger, J. S. Centrifugation speed affects light transmission aggregometry. Int J Lab Hematol 34, 81-85 (2012).
49 Refaai, M. & Laposta, M. Platelet Aggregaton. In: Michelson A D, ed. Platelets. San Diego, CA: Academic Press;2002:291-296.
50 Breddin, H. K. Can platelet aggregometry be standardized? Platelets 16, 151-158 (2005).
51 Born, G. V. Aggregation of blood platelets by adenosine diphosphate and its reversal. Nature 194, 927-929 (1962).
52 Marcantoni, E. et al. Platelet Transcriptome Profiling in HIV and ATP-Binding Cassette Subfamily C Member 4 (ABCC4) as a Mediator of Platelet Activity. JACC Basic Transl Sci 3, 9-22 (2018).
53 github.com/igordot/sns (2020).
54 Dobin, A. et al. STAR: ultrafast universal RNA-seq aligner. Bioinformatics 29, 15-21 (2013).
55 Liao, Y., Smyth, G. K. & Shi, W. featureCounts: an efficient general purpose program for assigning sequence reads to genomic features. Bioinformatics 30, 923-930 (2014).
56 Andrews. Babraham Bioinformatics-FastQC A Quality Control tool for High Throughput Sequence Data.
57 Love, M. I., Huber, W. & Anders, S. Moderated estimation of fold change and dispersion for RNA-seq data with DESeq2. Genome Biol 15, 550 (2014).
58 Myers, R. A. et al. Aspirin effects on platelet gene expression are associated with a paradoxical, increase in platelet function. Br J Clin Pharmacol 88, 2074-2083 (2022).
59 Liberzon, A. et al. Molecular signatures database (MSigDB) 3.0. Bioinformatics 27, 1739-1740 (2011).
60 Subramanian, A. et al. Gene set enrichment analysis: a knowledge-based approach for interpreting genome-wide expression profiles. Proc Natl Acad Sci USA 102, 15545-15550 (2005).
61 Yu, G., Wang, L. G., Han, Y. & He, Q. Y. clusterProfiler: an R package for comparing biological themes among gene clusters. OMICS 16, 284-287 (2012).
62 Kang, L., Chen, W., Petrick, N. A. & Gallas, B. D. Comparing two correlated C indices with right-censored survival outcome: a one-shot nonparametric approach. Stat Med 34, 685-703 (2015).
63 Pencina, M. J., D'Agostino, R. B., Sr., D'Agostino, R. B., Jr. & Vasan, R. S. Evaluating the added predictive ability of a new marker: from area under the ROC curve to reclassification and beyond. Stat Med 27, 157-172; discussion 207-112 (2008).
65 Thygesen K, Alpert J S, Jaffe A S, et al. Fourth Universal Definition of Myocardial Infarction (2018). Circulation 2018; 138 (20): e618-e651. DOI: 10.1161/CIR.0000000000000617.
66 Furie K L, Kasner S E, Adams R J, et al. Guidelines for the prevention of stroke in patients with stroke or transient ischemic attack: a guideline for healthcare professionals from the american heart association/american stroke association. Stroke 2011; 42 (1): 227-76. DOI: 10.1161/STR.0b013e3181f7d043.
67 Easton J D, Saver J L, Albers G W, et al. Definition and evaluation of transient ischemic attack: a scientific statement for healthcare professionals from the American Heart Association/American Stroke Association Stroke Council; Council on Cardiovascular Surgery and Anesthesia; Council on Cardiovascular Radiology and Intervention; Council on Cardiovascular Nursing; and the Interdisciplinary Council on Peripheral Vascular Disease. The American Academy of Neurology affirms the value of this statement as an educational tool for neurologists. Stroke 2009; 40 (6): 2276-93. DOI: 10.1161/STROKEAHA.108.192218.
68 Farber A, Menard M T, Conte M S, et al. Surgery or Endovascular Therapy for Chronic Limb-Threatening Ischemia. N Engl J Med 2022;387 (25): 2305-2316. DOI: 10.1056/NEJMoa2207899.
69 Berger J S, Katona B G, Jones W S, et al. Design and rationale for the Effects of Ticagrelor and Clopidogrel in Patients with Peripheral Artery Disease (EUCLID) trial. Am Heart J 2016; 175:86-93. DOI: 10.1016/j.ahj.2016.01.018.
70 Hess C N, Debus E S, Nehler M R, et al. Reduction in Acute Limb Ischemia With Rivaroxaban Versus Placebo in Peripheral Artery Disease After Lower Extremity Revascularization: Insights From VOYAGER PAD. Circulation 2021; 144 (23): 1831-1841. DOI: 10.1161/CIRCULATIONAHA.121.055146.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method for determining whether a subject has platelet hyperreactivity, the method comprising:
(a) determining in a blood sample collected from the subject, expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
(b) calculating a Platelet Reactivity Expression Score (PRESS) for the sample as a z-scored predictive probability based on weighted expression values calculated from the expression level of the genes determined in step (a); and
(c) determining that i) the subject has platelet hyperreactivity when the score is higher than a median PRESS score of a control cohort; or ii) the subject does not have platelet hyperreactivity when the score is lower than or equal to the median PRESS score of a control cohort.

2. The method of claim 1, wherein when the subject is determined to have platelet hyperreactivity, the method further comprises determining severity of platelet hyperreactivity in the subject based at least in part on the score calculated in step (b), wherein a higher score indicates more severe platelet hyperreactivity.

3. A method for determining risk of developing a platelet-mediated cardiovascular and/or limb event in a subject, the method comprising:
(a) determining in a blood sample collected from the subject, expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
(b) calculating a Platelet Reactivity Expression Score (PRESS) for the sample as a z-scored predictive probability based on weighted expression values calculated from the expression level of the genes determined in step (a); and
(c) determining that i) the subject is at risk of developing a cardiovascular and/or limb event when the score is higher than a median PRESS score of a control cohort; or ii) the subject is not at risk of developing a cardiovascular and/or limb event when the score is lower than or equal to the median PRESS score of a control cohort.

4. A method for determining whether a subject has a condition associated with platelet hyperreactivity, the method comprising:
(a) determining in a blood sample collected from the subject, expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
(b) calculating a Platelet Reactivity Expression Score (PRESS) for the sample as a z-scored predictive probability based on weighted expression values calculated from the expression level of the genes determined in step (a); and
(c) determining that i) the subject has a condition associated with platelet hyperreactivity when the score is higher than a median PRESS score of a control cohort; or ii) the subject does not have a condition associated with platelet hyperreactivity when the score is lower than or equal to the median PRESS score of a control cohort.

5. The method of any one of claims 1-4, wherein the method further comprises administering to the subject one or more antiplatelet treatments.

6. A method for preventing a cardiovascular and/or limb event in a subject in need thereof, the method comprising:
(a) determining in a blood sample collected from the subject expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
(b) calculating a Platelet Reactivity Expression Score (PRESS) for the sample as a z-scored predictive probability based on weighted expression values calculated from the expression level of the genes determined in step (a); and
(c) administering to the subject one or more antiplatelet treatments when the score is higher than a median PRESS score of a control cohort.

7. A method for treating or preventing a condition associated with platelet hyperreactivity in a subject in need thereof, the method comprising:
(a) determining in a blood sample collected from the subject, expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
(b) calculating a Platelet Reactivity Expression Score (PRESS) for the sample as a z-scored predictive probability based on weighted expression values calculated from the expression level of the genes determined in step (a); and
(c) administering to the subject one or more antiplatelet treatments when the score is higher than a median PRESS score of a control cohort.

8. The method of any one of claims 1-7, wherein the control cohort is a cohort of patients with peripheral artery disease (PAD).

9. The method of any one of claims 1-8, wherein the median PRESS score of a control cohort is about 0.38.

10. A method for monitoring the progression of a condition associated with platelet hyperreactivity in a subject diagnosed with said condition, the method comprising:
(a) determining in two or more blood samples collected from the subject at spaced apart time points expression level of the following genes AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
(b) calculating a Platelet Reactivity Expression Score (PRESS) for each sample as a z-scored predictive probability based on weighted expression values calculated from the expression level of the genes determined in step (a); comparing the scores calculated in step (b) between earlier and later collected samples; and
(c) determining that (i) the condition in the subject has progressed when the score is increased in the later collected sample(s) as compared to the earlier collected sample(s), or (ii) the condition in the subject has not progressed when the score is not increased in the later collected sample(s) as compared to the earlier collected sample(s).

11. A method for determining the effect of an antiplatelet treatment on development of a condition associated with platelet hyperreactivity in a subject diagnosed with said condition, the method comprising:
(a) determining in blood samples collected from the subject prior to and after administering the antiplatelet treatment, expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIPSK1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
(b) calculating a Platelet Reactivity Expression Score (PRESS) for each sample as a z-scored predictive probability based on weighted expression values calculated from the expression level of the genes determined in step (a);
(c) comparing the scores calculated in step (b) between samples collected from the subject prior to and after administering the antiplatelet treatment; and
(d) determining that (i) the antiplatelet treatment is effective when the score is decreased in the sample(s) collected after administering the antiplatelet treatment as compared to the sample(s) collected before administering the antiplatelet treatment, or (ii) the antiplatelet treatment is not effective when the score is the same or increased in the sample(s) collected after administering the antiplatelet treatment as compared to the sample(s) collected before administering the antiplatelet treatment.

12. The method of claim 11, comprising prior to step (a) the following steps:
(1) collecting one or more sample(s) from the subject prior to initiation of the antiplatelet treatment,
(2) administering the antiplatelet treatment to the subject, and
(3) collecting one or more sample(s) from the subject in the course of or following the antiplatelet treatment.

13. A method for identifying an antiplatelet treatment useful for slowing down the progression or treating a condition associated with platelet hyperreactivity in a subject diagnosed with said condition, the method comprising:
(a) determining in blood samples collected from the subject prior to and after administering a test antiplatelet treatment expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
(b) calculating a Platelet Reactivity Expression Score (PRESS) for each sample as a z-scored predictive probability based on weighted expression values calculated from the expression level of the genes determined in step (a);
(c) comparing the scores calculated in step (b) between samples collected from the subject prior to and after administering the test antiplatelet treatment; and
(d) determining that (i) the test antiplatelet treatment is effective for slowing down the progression or treating the condition when the score is decreased in the sample(s) collected after administering the test antiplatelet treatment as compared to the sample(s) collected before administering the test antiplatelet treatment, or (ii) the test antiplatelet treatment is not effective for slowing down the progression or treating the condition when the score is the same or increased in the sample(s) collected after administering the test antiplatelet treatment as compared to the sample(s) collected before administering the test antiplatelet treatment.

14. The method of claim 13, comprising prior to step (a) the following steps:
(1) collecting one or more sample(s) from the subject prior to administering the test antiplatelet treatment,
(2) administering the test antiplatelet treatment to the subject, and
(3) collecting one or more sample(s) from the subject after administering the test antiplatelet treatment.

15. The method of any one of claims 1-14, wherein step (a) comprises determining the expression level of additional genes selected from the genes listed in Table 9.

16. The method of claim 15, wherein step (a) comprises determining the expression level of at least 20, at least 50, at least 100, at least 200, or at least 300 additional genes selected from the genes listed in Table 9.

17. The method of claim 16, wherein step (a) comprises determining the expression level of all 451 genes listed in Table 9.

18. The method of any one of claims 1-17, wherein the genes are weighted according to the values in Table 10.

19. The method of any one of claims 1-18, wherein the expression level is determined by measuring mRNA level.

20. The method of claim 19, wherein the mRNA level is determined using RNA-seq, real-time polymerase chain reaction (RT-PCR), Northern blot analysis, or a Ribonuclease protection assay.

21. The method of any one of claims 1-20, wherein the blood sample comprises isolated platelets.

22. The method of any one of claims 1-21, wherein the blood sample consists of isolated platelets.

23. The method of any one of claims 1-25, 4-5, and 7-22, wherein the platelet hyperreactivity is measured as >60% aggregation in response to submaximal epinephrine (0.4 μM).

24. The method of any one of claims 4-5, and 7-23, wherein the condition associated with platelet hyperreactivity is cardiovascular disease, systemic lupus erythematosus (SLE), and COVID-19.

25. The method of any one of claims 1-9, wherein the subject has not been previously diagnosed with a cardiovascular disease.

26. The method of any one of claims 1-9, wherein the subject has been previously diagnosed with a cardiovascular disease.

27. The method of any one of claims 24-26, wherein the cardiovascular disease is atherosclerosis, peripheral artery disease (lower extremity atherosclerosis), or coronary artery disease.

28. The method of any one of claim 3, or 6, wherein the cardiovascular event is death, myocardial infarction (MI), and/or stroke.

29. The method of any one of claim 3, or 6, wherein the limb event is major amputation, acute limb ischemia, or a reintervention of the index limb.

30. The method of claim 29, wherein the reintervention of the index limb comprises a new bypass graft, a jump/interposition graft revision, thrombectomy/thrombolysis, balloon angioplasty, atherectomy, laser treatment and/or stenting or stent/grafting.

31. The method of any one of claims 5-30, wherein the antiplatelet treatment comprises one or more of aspirin, acadesine, prasugrel, ticagrelor, enilogrel, eptifibatide, cangrelor, anagrelide, anipamil, argatroban, clopidogrel, FR-122047, danaparoid sodium, dazoxiben hydrochloride, diadenosine 5′,5′″-P1,P4-tetraphosphate (Ap4A) analogs, defibrotide, dilazep dihydrochloride, 1,2- and 1,3-glyceryl dinitrate, dipyridamole, dopamine and 3-methoxytyramine, efegatran sulfate, enoxaparin sodium, glucagon, Ro-43-8857, L-700,462, ifetroban, ifetroban sodium, iloprost, isocarbacyclin methyl ester, isosorbide-5-mononitrate, itazigrel, ketanserin, BM-13.177, lamifiban, lifarizine, molsidomine, nifedipine, oxagrelate, prostaglandin E (PGE), lexipafant, prostacyclin (PGI2), pyrazines, pyridinol carbamate, abciximab, sulfinpyrazone, BN-50727, BN-52021, CV-4151, E-5510, FK-409, GU-7, KB-2796, KBT-3022, KC-404, KF-4939, OP-41483-, TRK-100, TA-3090, TFC-612 and ZK-36374, 2,4,5,7-tetrathiaoctane, 2,4,5,7-tetrathiaoctane 2,2-dioxide, 2,4,5-trithiahexane, theophylline, pentoxifylline, picotamide, sulotroban, tirofiban, trapidil, trifenagrel, trilinolein, dipyridamole, clofibrate, caffeine, ticlopidine, or an analog or derivative thereof.

32. The method of any one of claims 1-31, wherein the subject is human.

33. A non-transitory computer-readable medium configured to communicate with one or more processor(s) of a computing device, the non-transitory computer-readable medium including instructions thereon that, when executed by the processor(s), cause the computing device to perform the method of any one of claims 1-32.

34. A computing system comprising:
a memory;
one or more processors; and
program instructions stored in the memory that, when executed by the one or more processors, cause the computing system to:
(a) determine, in a blood sample collected from a subject, expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A; and
(b) calculate a Platelet Reactivity Expression Score (PRESS) for the blood sample as a z-scored predictive probability based on weighted expression values calculated from the expression level of the genes determined in process step (a).

35. The computing system of claim 34, further comprising instructions stored in the memory that, when executed by the one or more processors, cause the computing system to determine that
i) the subject has platelet hyperreactivity when the score is higher than a median PRESS score of a control cohort; or
ii) the subject does not have platelet hyperreactivity when the score is lower than or equal to the median PRESS score of a control cohort.

36. The computing system of claim 34, further comprising instructions stored in the memory that, when executed by the one or more processors, cause the computing system to determine that
i) the subject is at risk of developing a cardiovascular and/or limb event when the score is higher than a median PRESS score of a control cohort; or
ii) the subject is not at risk of developing a cardiovascular and/or limb event when the score is lower than or equal to the median PRESS score of a control cohort.

37. The computing system of claim 34, further comprising instructions stored in the memory that, when executed by the one or more processors, cause the computing system to determine that
i) the subject has a condition associated with platelet hyperreactivity when the score is higher than a median PRESS score of a control cohort; or
ii) the subject does not have a condition associated with platelet hyperreactivity when the score is lower than or equal to the median PRESS score of a control cohort.

38. The computing system of claim 34, further comprising:
an input/output (I/O) device; and
instructions stored in the memory that, when executed by the one or more processors, cause the computing system to:
(a) output, for display on the I/O device, a recommendation to administer, to the subject, one or more antiplatelet treatments when the score is higher than a median PRESS score of a control cohort.

39. A computing system comprising:
a memory;
one or more processors; and
program instructions stored in the memory that, when executed by the one or more processors, cause the computing system to calculate a Platelet Reactivity Expression Score (PRESS) for a blood sample collected from a subject,
wherein the PRESS is calculated as a z-scored predictive probability based on weighted expression values calculated from the expression level of the following genes in the blood sample: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A.

40. The computing system of claim 39, further comprising instructions stored in the memory that, when executed by the one or more processors, cause the computing system to determine that
i) the subject has platelet hyperreactivity when the score is higher than a median PRESS score of a control cohort; or
ii) the subject does not have platelet hyperreactivity when the score is lower than or equal to the median PRESS score of a control cohort.

41. The computing system of claim 39, further comprising instructions stored in the memory that, when executed by the one or more processors, cause the computing system to determine that
i) the subject is at risk of developing a cardiovascular event when the score is higher than a median PRESS score of a control cohort; or
ii) the subject is not at risk of developing a cardiovascular event when the score is lower than or equal to the median PRESS score of a control cohort.

42. The computing system of claim 39, further comprising instructions stored in the memory that, when executed by the one or more processors, cause the computing system to determine that
i) the subject has a condition associated with platelet hyperreactivity when the score is higher than a median PRESS score of a control cohort; or
ii) the subject does not have a condition associated with platelet hyperreactivity when the score is lower than or equal to the median PRESS score of a control cohort.

43. The computing system of claim 39, further comprising:
an input/output (I/O) device; and
instructions stored in the memory that, when executed by the one or more processors, cause the computing system to:
(a) output, for display on the I/O device, a recommendation to administer, to the subject, one or more antiplatelet treatments when the score is higher than a median PRESS score of a control cohort.

44. A computing system for monitoring the progression of a condition associated with platelet hyperreactivity in a subject diagnosed with said condition, comprising:
a memory;
one or more processors; and
program instructions stored in the memory that, when executed by the one or more processors, cause the computing system to:
(a) determine in two or more blood samples collected from the subject at spaced apart time points expression level of the following genes AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBPIP2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A;
(b) calculate a Platelet Reactivity Expression Score (PRESS) for each sample as a z-scored predictive probability based on weighted expression values of the genes calculated from the expression level determined in process step (a); and
(c) compare the scores calculated in process step (b) between earlier and later collected samples.

45. The computing system of claim 44, further comprising instructions stored in the memory that, when executed by the one or more processors, cause the computing system to determine that
(i) the condition in the subject has progressed when the score is increased in the later collected sample(s) as compared to the earlier collected sample(s), or
(ii) the condition in the subject has not progressed when the score is not increased in the later collected sample(s) as compared to the earlier collected sample(s).

46. A computing system for determining the effect of an antiplatelet treatment on development of a condition associated with platelet hyperreactivity in a subject diagnosed with said condition, comprising:
a memory;
one or more processors; and
program instructions stored in the memory that, when executed by the one or more processors, cause the computing system to:
(a) determine, in blood samples collected from the subject prior to and after administering the antiplatelet treatment, expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A; and
(b) calculate a Platelet Reactivity Expression Score (PRESS) for each sample as a z-scored predictive probability based on weighted expression values calculated from the expression level of the genes determined in process step (a).

47. The computing system of claim 46, further comprising instructions stored in the memory that, when executed by the one or more processors, cause the computing system to compare the scores calculated in process step (b) between samples collected from the subject prior to and after administering the antiplatelet treatment, determine that
(i) the antiplatelet treatment is effective when the score is decreased in the sample(s) collected after administering the antiplatelet treatment as compared to the sample(s) collected before administering the antiplatelet treatment, or
(ii) the antiplatelet treatment is not effective when the score is the same or increased in the sample(s) collected after administering the antiplatelet treatment as compared to the sample(s) collected before administering the antiplatelet treatment.

48. A computing system for identifying an antiplatelet treatment useful for slowing down the progression or treating a condition associated with platelet hyperreactivity in a subject diagnosed with said condition, comprising:
a memory;
one or more processors; and
program instructions stored in the memory that, when executed by the one or more processors, cause the computing system to:
(a) determine in blood samples collected from the subject prior to and after administering a test antiplatelet treatment expression level of the following genes: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1L4, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A; and
(b) calculate a Platelet Reactivity Expression Score (PRESS) for each sample as a z-scored predictive probability based on weighted expression values calculated from the expression level of the genes determined in process step (a).

49. The computing system of claim 48, further comprising instructions stored in the memory that, when executed by the one or more processors, cause the computing system to:
compare the scores calculated in process step (b) between samples collected from the subject prior to and after administering the test antiplatelet treatment; and determine that
(a) the test antiplatelet treatment is effective for slowing down the progression or treating the condition when the score is decreased in the sample(s) collected after administering the test antiplatelet treatment as compared to the sample(s) collected before administering the test antiplatelet treatment, or
(b) the test antiplatelet treatment is not effective for slowing down the progression or treating the condition when the score is the same or increased in the sample(s) collected after administering the test antiplatelet treatment as compared to the sample(s) collected before administering the test antiplatelet treatment.

50. A computing system comprising:
a memory;
one or more processors; and
program instructions stored in the memory that, when executed by the one or more processors, cause the computing system to:
calculate a first value of a subject as a z-scored predictive probability based on weighted expression values calculated from an expression level of a subset of genes; and
output a recommendation to administer, to the subject, one or more antiplatelet treatments when the value is higher than a median value score of a control cohort, wherein the expression level is determined for the following genes in a blood sample of the subject: AC006378.2, AC087481.3, AC106795.2, AC112200.2, AC131971.1, AC243829.1, ACTR3C, AL589743.1, ALCAM, AP1B1, ARHGAP10, ARV1, BCR, BMT2, BX537318.1, C20orf96, CA7, CCNY, CDYL2, CIZ1, COQ5, CRYBA4, CTSA, DDX3Y, DISP1, DYRK4, EVL, FAM189B, FBXL13, GPX7, HSBP1P2, HSPA2, IMPA2, IPCEF1, IQUB, KANSL1L, KCTD2, KIF3A, LAMC1, MCEMP1, MGST2, MICA, MYLK, MYO18B, NAP1LA, NBEAP1, NOMO2, NR1D2, PAPPA, PIP5K1B, PTMAP10, RAB3GAP2, RAC1P4, RAD51AP1, RASA1, RTN1, SEC22A, SEM1, SEPT7P7, SLC3A2, SPARC, SPIN4, SUGTIP1, SVIL, TCAIM, THNSL1, THUMPD2, TRBV4-1, TSPAN13, UBE4B, VNN1, ZBTB11, ZFR, ZHX1-C8orf76, ZNF460-AS1, and ZNF804A.

51. The computing system of claim 50, wherein the first value is a Platelet Reactivity Expression Score (PRESS).
